(19) United States
(12) Patent Application Publication
(10) Pub. No.: US 2022/0298130 A1
(43) Pub. Date: Sep. 22, 2022

(54) ORGANIC ELECTRONIC DEVICE

(71) Applicant: DUK SAN NEOLUX CO., LTD., Cheonan-si, Chungcheongnam-do (KR)

(72) Inventors: Soung Yun MUN, Cheonan-si (KR); Min Ji JO, Cheonan-si (KR); Chi Hyun PARK, Cheonan-si (KR); Yong Wook PARK, Cheonan-si Chungcheongnam-do (KR); Sun Hee LEE, Cheonan-si Chungcheongnam-do (KR)

(21) Appl. No.: 17/631,552

(22) PCT Filed: Jul. 30, 2020
(86) PCT No.: PCT/KR2020/010043

§ 371 Date: Jan. 31, 2022

(30) Foreign Application Priority Data
Aug. 2, 2019 (KR) 10-2019-0094551
Apr. 20, 2020 (KR) 10-2020-0047662
Jul. 3, 2020 (KR) 10-2020-0082255

(51) Int. Cl. C07D 307/91; C07D 209/82; C07D 333/76; C07C 211/54; C07D 401/12; C07D 405/12; C07D 409/14; C07D 403/12; C07D 409/12; C07D 495/04; H01L 51/00

(52) U.S. Cl. CPC C07D 307/91; C07D 209/82; C07D 333/76; C07C 211/54; C07D 401/12; C07D 405/12; C07D 409/14; C07D 403/12; C07D 409/12; C07D 495/04; H01L 51/0069; H01L 51/0071; H01L 51/0072; H01L 51/0073; H01L 51/0074; H01L 51/0062; H01L 51/506

(57) ABSTRACT

Embodiments of the present invention relate to an organic electronic device capable of ensuring high luminous efficiency, low driving voltage and high heat resistance, and improving color purity or lifespan.

### TECHNICAL FIELD

Embodiments of the present disclosure relate to an organic electronic device.

### BACKGROUND ART

In general, organic electroluminescence refers to a phenomenon in which electrical energy is converted into light energy using an organic material. An organic electronic device using organic electroluminescence has a structure generally including an anode, a cathode, and an organic material layer positioned between the anode and the cathode. The organic material layer has a multilayer structure comprised of a plurality of layers formed of different materials to improve the efficiency and stability of the organic electronic device.

Currently, in the portable display market, displays are increasing in size to be large-area displays. Since portable displays are provided with a battery serving as a limited power source, portable displays require more efficient power consumption than that required by conventional portable displays. In addition, in this situation, not only the challenge for efficient power consumption but also challenges related to luminous efficiency and lifespan need to be solved.

In order to overcome the problems related to the power consumption, luminous efficiency, and lifespan, research on a tandem organic electronic device in which the organic material layer includes two or more stacks (or emission units) each including an emission layer has been undertaken. In particular, research for improving the power consumption, luminous efficiency, and lifespan by improving the organic material included in the stacks has been undertaken.

Efficiency, lifespan, a driving voltage, and the like are related to each other. An increase in efficiency leads to a relative decrease in driving voltage, by which the crystallization of the organic material due to Joule heating during driving may be reduced, thereby increasing the lifespan. However, simply improving the organic material layer may not maximize efficiency. This is because, when the optimal combination of the energy level and T1 value between each organic material and the intrinsic properties (e.g., mobility, interfacial properties) of the material are achieved, both increased lifespan and high efficiency may be achieved. Therefore, it is necessary to develop a material that may efficiently achieve charge balance in an emission layer while having high thermal stability.

In particular, in a tandem organic electronic device, the efficiency, lifespan, and driving voltage of the organic electronic device may vary depending on which organic materials are combined and used in specific layers.

### DISCLOSURE

### Technical Problem

Embodiments of the present disclosure may provide an organic electronic device having a low driving voltage, high efficiency, high color purity, and increased lifespan.

### Technical Solution

In one aspect, an organic electronic device according to embodiments of the present disclosure includes a first electrode, a second electrode, and an organic material layer.

The organic material is positioned between the first electrode and the second electrode, and includes a first stack, a second stack, and a third stack.

The first stack includes a first hole transport region, a first emission layer, and a first electron transport region.

The first hole transport region includes a first hole transport layer and a first auxiliary emission layer.

The first hole transport layer or the first auxiliary emission layer includes a first compound represented by the following Formula 1.

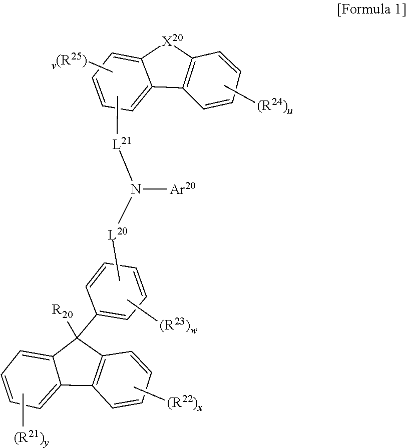

### Advantageous Effects

Embodiments of the present disclosure may provide an organic electronic device in which high luminous efficiency, a low driving voltage, high thermal resistance, significantly improved color purity, and significantly increased lifespan are realized.

### DESCRIPTION OF DRAWINGS

FIG. 1 is a diagram schematically illustrating an organic electronic device according to embodiments of the present disclosure;

FIG. 2 is a diagram schematically illustrating the first hole transport layer of the organic electronic device according to embodiments of the present disclosure;

FIGS. 3 and 4 are diagrams schematically illustrating the organic electronic device according to embodiments of the present disclosure; and

FIG. 5 is a diagram schematically illustrating a stack of an organic electronic device according to embodiments of the present disclosure.

### BEST MODE

Hereinafter, embodiments of the present disclosure will be described in detail with reference to the illustrative drawings.

In designating elements of the drawings by reference numerals, the same elements will be designated by the same reference numerals although they are shown in different drawings. Further, in the following description of the present disclosure, a detailed description of known functions and configurations incorporated herein will be omitted in the case in which the subject matter of the present disclosure may be rendered unclear thereby. It will be understood that the terms “comprise”, “have”, “consist of”, and any variations thereof used herein are intended to cover non-exclusive inclusions unless explicitly stated to the contrary. Descriptions of elements in the singular form used herein are intended to include descriptions of elements in the plural form, unless explicitly stated to the contrary.

In addition, terms, such as first, second, A, B, (a), or (b), may be used herein when describing elements of the present disclosure. Each of these terminologies is not used to define an essence, order, or sequence of a corresponding element but used merely to distinguish the corresponding element from other elements.

It will be understood that when an element is referred to as being “connected”, “coupled”, or “joined” to another element, not only can it be “directly connected, coupled, or joined” to the other element, but it can also be “indirectly connected, coupled, or joined” to the other element via an “intervening” element. Here, the intervening element may be included in one or more of the two elements “connected”, “coupled”, or “joined” to each other.

In addition, it will be understood that when an element, such as a layer, a film, or a region, or a plate, is referred to as being “above” or “on” another element, not only can it be “directly” above or on the other element, but it can also be “indirectly” above or on the other element or layer via an “intervening” element. In contrast, when an element is referred to as being “directly” above or on another element, it will be understood that no intervening element is interposed.

When time relative terms, such as “after”, “subsequent to”, “next”, “before”, and the like, are used to describe elements, operating or manufacturing methods, and the like, these terms may be used to describe non-consecutive or non-sequential processes or operations unless the term “directly” or “immediately” is used together.

In addition, when any numerical values for elements or corresponding information are mentioned, it should be considered that numerical values for elements or corresponding information include a tolerance or error range that may be caused by various factors (e.g., process factors, internal or external impact, noise, etc.) even when a relevant description is not specified.

Unless otherwise stated, the term “halo” or “halogen”, as used herein, refers to fluorine (F), chlorine (Cl), bromine (Br), iodine (I), or the like.

Unless otherwise stated, the term “alkyl” or “alkyl group”, as used herein, may have a single bond of 1 to 60 carbon atoms, and refer to saturated aliphatic functional radicals including a straight chain alkyl group, a branched chain alkyl group, a cycloalkyl (alicyclic) group, an alkyl-substituted cycloalkyl group, or a cycloalkyl-substituted alkyl group.

Unless otherwise stated, the term “haloalkyl” or “halogen alkyl”, as used herein, may include a halogen-substituted alkyl group.

Unless otherwise stated, the term “alkenyl” or “alkynyl”, as used herein, may have a double or triple bond of 2 to 60 carbon atoms and include a straight chain group or a branched chain group.

Unless otherwise stated, the term “cycloalkyl” as used herein may refer to alkyl forming a ring having 3 to 60 carbon atoms.

The term “alkoxy group” or “alkyloxy group”, as used herein, refers to an alkyl group to which an oxygen radical is bonded and, unless otherwise stated, may have 1 to 60 carbon atoms.

The term “alkenoxyl group”, “alkenoxy group”, “alkenyloxyl group”, or “alkenyloxy group” refers to an alkenyl group to which an oxygen radical is attached, and unless otherwise stated, may have 2 to 60 carbon atoms.

Unless otherwise stated, the term “aryl group” or “arylene group”, as used herein, has, but is not limited thereto, 6 to 60 carbon atoms. Herein, the aryl group or the arylene group may include a monocyclic compound, a ring assembly, fused polycyclic systems, a spiro compound, or the like. For example, the aryl group includes, but is not limited to, a phenyl group, biphenyl, naphthyl, anthryl, indenyl, phenanthryl, triphenylenyl, pyrenyl, peryleneyl, chrysenyl, naphthacenyl, fluoranthenyl, and the like. Naphthyl may include 1-naphthyl and 2-naphthyl, and anthryl may include 1-anthryl, 2-anthryl, and 9-anthryl.

Unless stated otherwise, the term “fluorenyl group” or “fluorenylene group”, as used herein, may refer to a monovalent or divalent functional group of fluorene. In addition, the “fluorenyl group” or “fluorenylene group” may refer to a substituted fluorenyl group or a substituted fluorenylene group. The “substituted fluorenyl group” or the “substituted fluorenylene group” may refer to a monovalent or divalent functional group of substituted fluorene. The term “substituted fluorene” may refer to a compound in which at least one of substituent R, R′, R″, or R′″ below is a functional group other than hydrogen, and include a case in which R and R′ are bonded to each other to form a spiro compound together with carbon atoms bonded thereto.

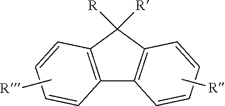

The term “spiro compound”, as used herein, has “a spiro union”, which refers to a union of two rings sharing only one atom. In this case, the atom shared by the two rings is referred to as a “spiro atom”. Such spiro compounds are referred to, for example, as “monospiro”, “dispiro”, and “trispiro” compounds depending on the number of spiro atoms included in the compound.

The term “heterocyclic group”, as used herein, includes not only aromatic rings, such as a “heteroaryl group” or a “heteroarylene group”, but also non-aromatic rings, and unless stated otherwise, refers to, but is not limited to, monocyclic and multicyclic rings each including one or more heteroatoms and having 2 to 60 carbon atoms. The term “heteroatom”, as used herein, refers to N, O, S, P, or Si, unless stated otherwise. The “heterocyclic group” may refer to monocyclic compounds, ring assemblies, fused polycyclic systems, spiro compounds, or the like including heteroatoms.

In addition, the “heterocyclic group”, as used herein, may include rings having SO₂ in place of a ring-forming carbon atom. For example, the “heterocyclic group” may include the following compound.

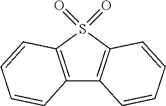 [SMILES: O=S1(=O)c2ccccc2-c2ccccc21]

The term “ring”, as used herein, may refer to monocyclic rings and polycyclic rings, include not only hydrocarbon rings but also hetero rings including at least one heteroatom, and include aromatic rings and non-aromatic rings.

The term “polycyclic ring”, as used herein, may include ring assemblies, fused polycyclic systems, and spiro compounds. The polycyclic ring may include not only aromatic compounds but also non-aromatic compounds, and include not only hydrocarbon rings but also hetero rings including at least one heteroatom.

The term “aliphatic cyclic group”, as used herein, may refer to cyclic hydrocarbons except for aromatic hydrocarbons, include single rings, ring assemblies, fused ring systems, spiro compounds, and the and unless stated otherwise, mean rings each having 3 to 60 carbon atoms. For example, a fused system of benzene which is an aromatic ring and cyclohexane is a non-aromatic ring corresponds to an aliphatic ring.

The term “ring assembly”, as used herein, refers to a compound in which two or more rings (single rings or fused ring systems) are connected directly by a single or double bond. For example, in the aryl group, the ring assembly may be, but is not limited to, a biphenyl group, a terphenyl group, or the like.

The term “fused polycyclic system”, as used herein, refers to a form of fused rings sharing at least two atoms. For example, in the aryl group, the fused polycyclic system may be, but is not limited to, naphthalenyl group, a phenanthrenyl group, a fluorenyl group, or the like.

In addition, in the case that prefixes are named consecutively, this means that substituents are listed in the order of the prefixes. For example, an aryl alkoxy group may refer to an alkoxy group substituted with an aryl group, an alkoxy carbonyl group may refer to a carbonyl group substituted with an alkoxy group, and an aryl carbonyl alkenyl group may refer to an alkenyl group substituted with an arylcarbonyl group. Here, the arylcarbonyl group may be a carbonyl group substituted with an aryl group.

Unless clearly stated otherwise, the term “substituted” in the term “substituted or non-substituted”, as used herein, may refer to, but is not limited to, deuterium, a halogen, an amino group, a nitrile group, a nitro group, a C₁-C₂₀ alkyl group, a C₁-C₂₀ alkoxy group, a C₁-C₂₀ alkyl amine group, a C₁-C₂₀ alkylthiophene group, a C₆-C₂₀ arylthiophene group, a C₂-C₂₀ alkenyl group, a C₂-C₂₀ alkynil group, a C₃-C₂₀ cycloalkyl group, a C₆-C₂₅ aryl group, a C₆-C₂₅ aryl group substituted with deuterium, a C₈-C₂₀ aryl alkenyl group, a silane group, a boron group, a germanium group, and a C₂-C₂₀ heterocyclic group including at least one heteroatom selected from the group consisting of O, N, S, Si, or P.

Herein, “the name of a functional group” corresponding to the aryl group, the arylene group, the heterocyclic group, or the like illustrated as each symbol and a substituent thereof may be written in “the name of the functional group on which the valence thereof is reflected” or may be written in “the name of the parent compound thereof”. For example, phenanthrene, i.e., a type of aryl group, may be written in group names by distinguishing the valence. That is, a monovalent phenanthrene “group” may be written as “phenanthryl (group)”, while a divalent phenanthrene “group” may be written as “phenanthrylene (group)”. Alternatively, the phenanthrene groups may be written as “phenanthrene”, i.e. the name of the parent compound, regardless of the valence. Similarly, pyrimidine may be written as “pyrimidine” regardless of the valence or may be written in group names each corresponding to the valence, in which a monovalent pyrimidine group is written as pyrimidinyl (group) and a divalent pyrimidine group is written as pyrimidinylen (group). Accordingly, when the type of a substituent is written in the name of the parent compound herein, the written name may refer to an n-valence “group” formed by the desorption of a carbon atom and/or a heteroatom-bonded hydrogen atom from the parent compound.

In addition, unless clearly stated otherwise, formulas used herein may be applied in the same manner as the definition of the substituent based on the exponential definition of the following Formula.

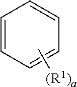

Here, when a is 0, the substituent R₁ is absent. This means that all hydrogens are bonded to carbons of the benzene ring. In this case, hydrogen bonded to carbon may not be shown, and the chemical Formula or compound may be described. When a is 1, one substituent R¹ is bonded to any one of carbon atoms of the benzene ring. When a is 2 or 3, substituents R¹ may respectively be combined as follows. When a is 4 to 6, substituents R¹ may be bonded to carbon atoms of the benzene ring. When a is an integer equal to or greater than 2, R¹s may be the same or different.

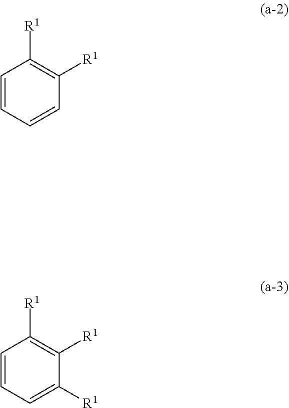

Herein, the substituents bonded to form rings, respectively, means that adjacent groups are bonded to each other to form a single ring or two or more fused rings. The single ring or the two or more fused rings formed in this manner may also include a hetero ring including at least one hetero atom, and include an aromatic ring and a non-aromatic ring.

Herein, the organic electronic device may refer to one or more compounds between an anode and a cathode, or an organic light-emitting diode (OLED) including the anode, the cathode, and the one or more compounds positioned between the anode and the cathode.

In addition, herein, ln some cases, the organic electronic device may refer to an OLED and a panel on which the OLED is provided, or an electronic apparatus including the panel and a circuit. For example, the electronic apparatus may be, but is not limited to, a display device, an illumination device, a solar cell, a portable or mobile terminal (e.g., a smartphone, a tablet computer, a personal digital assistant (PDA), an electronic dictionary, or a portable media player (PMP)), a navigation terminal, a game machine, various TVs, and various computer monitors. The electronic apparatus may be any type of apparatus including the above-described component(s).

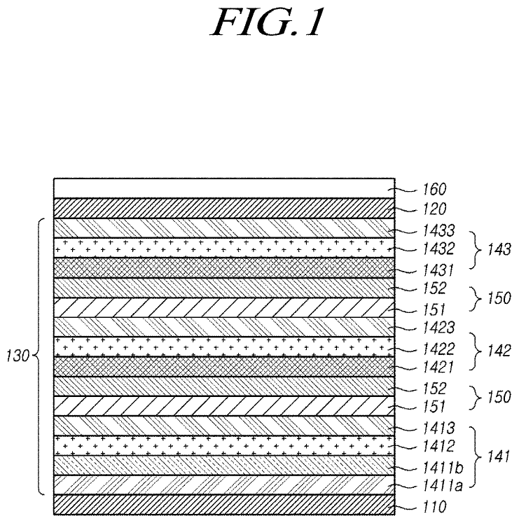

FIG. 1 is a diagram schematically illustrating an organic electronic device according to embodiments of the present disclosure.

An organic electronic device 100 according to embodiments includes a first electrode 110, a second electrode 120, and an organic material layer 130 positioned between the first electrode 110 and the second electrode 120 and including a first stack 141, a second stack 142, and a third stack 143.

Although FIG. 1 illustrates embodiments in which the second stack 142 is positioned above the first stack. 141 and the third stack 143 is positioned above the second stack 142, embodiments of the present disclosure are not limited thereto.

For example, the first electrode 110 may be an anode, whereas the second electrode 120 may be a cathode. The organic material layer 130 is a layer positioned between the first electrode 110 and the second electrode 120 and including an organic material. The organic material layer 130 may be comprised of a plurality of layers.

In an example, the first electrode 110 may be a transparent electrode, whereas the second electrode 120 may be a reflecting electrode. In another example, the first electrode 110 may be a reflecting electrode, whereas the second electrode 120 may be a transparent electrode.

Since the organic material layer 130 includes at least three stacks, the organic electronic device according to embodiments may be, for example, a tandem organic electronic device including a plurality of stacks. The organic material layer may be realized by repeatedly stacking the same stack three or more times stacking three or more different stacks.

The above-described three or more stacks may include the first stack 141, the second stack 142, and the third stack 143.

The first stack 141 includes a first hole transport region 1411, a first emission layer 1412, and a first electron transport region 1413.

The first emission layer 1412 is a layer emitting light energy generated by electron-hole recombination. For example, the first emission layer 1412 may include a host material and a dopant.

The first hole transport region 1411 may be, for example, a region positioned between the first electrode 110 serving as an anode and the first emission layer 1412 to transport holes from the first electrode 110 to the first emission layer 1412. The first electron transport region 1413 may be, for example, a region positioned between the second electrode 120 serving as cathode and the first emission layer 1412 to transport electrons from the second electrode 120 to the emission layer.

The first hole transport region 1411 may include a P-type dopant, whereas the first electron transport region 1413 may include an N-type dopant. Here, a P-doped layer refers to a layer doped with a P-type dopant to have more positive properties (i.e., the properties of holes) than before. In contrast, an N-doped layer refers to a layer doped with an N-dopant to have more negative properties (i.e., the properties of electrons) than before.

The thickness of the first hole transport region 1411 may be from 10 nm to 100 nm. The lower t of the thickness of the first hole transport region 1411 may be, for example, 15 nm or more or 20 nm or more. The upper limit of the thickness of the first hole transport region 1411 may be, for example, 90 nm or less or 80 nm or less. When the thickness of the first hole transport region 1411 is within this range, the organic electronic device may have high luminous efficiency, a low driving voltage, and increased lifespan.

The organic material layer 130 may include one or more charge generation layers 150 positioned between the stacks. The charge generation layers 150 refer to layers generating holes and electrons when a voltage is applied thereto. When three or more stacks are provided, the charge generation layers 150 may be positioned between the stacks. Here, the plurality of charge generation layers 150 may be the same as or different from each other. Since the charge generation layers 150 are disposed between the stacks, the current efficiency of each of the stacks can be increased and electric charges can be properly distributed over the stacks.

Specifically, each of the charge generation layers 150 may be provided between two adjacent stacks and serve to drive a tandem organic light-emitting device using only a pair of an anode and a cathode without separate internal electrodes positioned between the stacks.

The charge generation layers 150 may include, for example, an N-type charge generation layer 151 and a P-type charge generation layer 152. For example, the N-type charge generation layer 151 may be positioned adjacent to the first electrode 110 serving as an anode, whereas the P-type charge generation layer 152 may be positioned adjacent to the second electrode 120 serving as a cathode.

A capping layer 160 may be positioned above the second electrode 120. When the capping layer 160 is formed, the optical efficiency of the organic electronic device may be improved.

In a top emission organic electronic device, the capping layer 160 may serve to reduce optical energy loss in the second electrode 120 caused by surface plasmon polaritons (SPPs). In a bottom emission organic electronic device, the capping layer 160 may serve to buffer the second electrode 120.

The first hole transport region 1411 includes a first hole transport layer 1411a and a first auxiliary emission layer 1411b. The first auxiliary emission layer 1411b may be positioned between, for example, the first emission layer 1412 and the first hole transport layer 1411a.

The first electron transport region 1413 may include an electron transport layer (not shown).

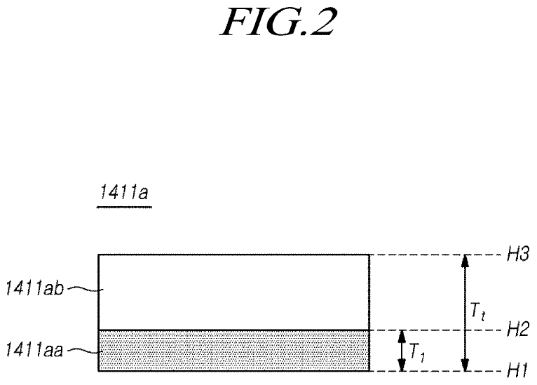

FIG. 2 is a diagram schematically illustrating the first hole transport layer 1411a of the organic electronic device according to embodiments of the present disclosure.

Referring to FIG. 2, the thickness Tt of the first hole transport layer 1411a may be defined as the distance between H1 and H3. H1 may be a boundary between the first hole transport layer 1411a and any layer positioned below the first hole transport layer, for example, the first electrode. H3 may be a boundary between the first hole transport layer 1411a and any layer positioned above the first hole transport layer, for example, the first auxiliary emission layer 1411b.

The thickness Tt of the first hole transport layer 1411a may be from 250 Å to 700 Å. The lower limit of the thickness Tt of the first hole transport layer 1411a may be, for example, 260 Å or more or 270 Å or more. The upper limit of the thickness Tt of the first hole transport layer 1411a may be, for example, 650 Å or less or 600 Å or less.

When the thickness of the first hole transport layer 1411a meets the above-described range, the first hole transport layer 1411a may include a hole transport material in an amount sufficient to have superior hole injection and transport functions while preventing electric charges from being excessively injected, thereby providing an organic electronic device having a low thickness while being superior in terms of driving voltage, efficiency, or lifespan.

In the first hole transport layer 1411a, 10% to 50% of the thickness Tt of the first hole transport layer may be doped with a first doping material. The portion of the first hole transport layer 1411a doped with the first doping material may be referred to as a first doping material-doped layer 1411aa. The first hole transport layer 1411a may include the first doping material-doped layer 1411aa doped with the first doping material and a first doping material undoped layer 1411ab not doped with the first doping material. The first doping material undoped layer 1411ab may be positioned between the first doping material-doped layer 1411aa and the first emission layer.

For example, the first hole transport layer 1411a may include a hole transport material. The first doping material-doped layer 1411aa may be a layer including the first doping material in addition to the hole transport material. The hole transport material is not particularly limited as long as it is a material having hole transport properties. For example, the hole transport material may be at least one selected from a first compound or a fourth compound.

The thickness T₁ of the first doping material-doped layer 1411aa may be 10% to 50% of the thickness of the first hole transport layer 1411a. The thickness T₁ of the first doping material-doped layer 1411aa may be defined as the distance between H1 and H2. H2 may be a boundary between the first doping material-doped layer 1411aa and the first doping material undoped layer 1411ab.

The lower limit of the ratio of the thickness T₁ of the first doping material-doped layer 1411aa with respect to the thickness of the first hole transport layer 1411a may be, for example, 12% or more or 15% or more. The upper limit of the ratio of the thickness T₁ of the first doping material-doped layer with respect to the thickness Tt of the first hole transport layer 1411a may be, for example, 40% or less or 30% or less.

The thickness T₁ of the first doping material-doped layer 1411aa may be, for example, from 30 Å to 300 Å while meeting the above-described range of the ratio with respect to the thickness Tt of the first hole transport layer. The lower limit of the thickness T₁ of the first doping material-doped layer 1411aa may be, for example, 60 Å or more or 80 Å or more, whereas the upper limit of the thickness T₁ of the first doping material-doped layer 1411aa may be, for example, 200 Å or less or 150 Å or less.

When the thickness T₁ of the first doping material-doped layer 1411aa meets the above-described ranges of the ratio and the thickness, the generation of holes and electric charges in the first hole transport layer 1411a may be promoted to facilitate the injection of holes into the first emission layer 1412, thereby providing an organic electronic device superior in terms of lifespan or efficiency. It is thus possible to prevent the problem of short circuits in the device and prevent fabrication costs from being increased through excessive use of the doping material.

The first doping material-doped layer may include the first compound, and include 5 to 15 parts by weight of the first doping material with respect to 100 parts by weight of the first compound.

The first doping material-doped layer may include at least one of the first compound or the fourth compound, and include 5 to 15 parts by weight of the first doping material with respect to 100 parts by weight of a total amount of the first compound and the fourth compound. The lower limit of the doping ratio of the first doping material may be, for example, 7 parts by weight or more or 9 parts by weight or more. The upper limit of the doping ratio of the first doping material may be, for example, 13 parts by weight or less or 11 parts by weight or less.

When the doping ratio of the first doping material meets the above-described range, the generation of holes and electric charges in the first hole transport layer may be promoted to facilitate the injection of holes into the first emission layer, thereby providing an organic electronic device superior in terms of lifespan or efficiency. It is thus possible to prevent the problem of short circuits in the device and prevent fabrication costs from being increased by excessive use of the doping material.

For the second stack 142 and the third stack 143, what has been described above for the first stack 141 may equally be applied, unless clearly stated otherwise.

The second stack 142 may include a second hole transport region, a second emission layer, and a second electron transport region. Regarding the second hole transport region, the second emission layer, and the second electron transport region, what has been described above for the first hole transport region 1411, the first emission layer 1412, and the first electron transport region 1413 may equally be applied, unless clearly stated otherwise.

The second hole transport region may include a second hole transport layer and a second auxiliary emission layer. Regarding the second hole transport layer and the second auxiliary emission layer, what has been described above for the first hole transport layer 1411a and the first auxiliary emission layer 1411b may equally be applied, unless clearly stated otherwise.

Regarding the thickness and the doping of the second hole transport layer, what has been described above for the thickness and the doping of the first hole transport layer 1411a may equally be applied.

The thickness of the second hole transport layer may be from 250 Å to 700 Å. The lower limit of the thickness of the second hole transport layer may be, for example, 260 Å or more or 270 Å or more. The upper limit of the thickness of the second hole transport layer may be, for example, 650 Å or less or 600 Å or less.

When the thickness of the second hole transport layer meets the above-described range, the second hole transport layer may include a hole transport material in an amount sufficient to have superior hole injection and transport functions and prevent electric charges from being excessively injected, thereby providing an organic electronic device having a low thickness while being superior in terms of driving voltage, efficiency, or lifespan.

In the second hole transport layer, 10% to 50% of the thickness of the second hole transport layer may be doped with a second doping material. The portion of the second hole transport layer doped with the second doping material may be referred to as a second doping material-doped layer. The second hole transport layer may include the second doping material-doped layer doped with the second doping material and a second doping material undoped layer not doped with the second doping material. The second doping material undoped layer may be positioned between the second doping material-doped layer and the second emission layer.

For example, the second hole transport layer may include a hole transport material. The second doping material-doped layer may be a layer including the second doping material in addition to the hole transport material. The transport material is not particularly limited as long as it is a material having hole transport properties. For example, the hole transport material may be a second compound.

The thickness of the second doping material-doped layer may be 10% to 50% of the thickness of the second hole transport layer. The lower limit of the ratio of the thickness of the second doping material-doped layer with respect to the thickness of the second hole transport layer may be, for example, 12% or more or 15% or more. The upper limit of the ratio of the thickness of the second doping material-doped layer with respect to the thickness of the second hole transport layer may be, for example, 40% or less or 30% or less.

The thickness of the second doping material-doped layer may be, for example, from 30 Å to 300 Å while meeting the above-described range of the ratio with respect to the thickness of the second hole transport layer. The lower limit of the thickness of the second doping material-doped layer may be, for example, 60 Å or more or 80 Å or more, whereas the upper limit of the thickness of the second doping material-doped layer may be, for example, 200 Å or less or 150 Å or less.

When the thickness of the second doping material-doped layer meets the above-described ranges of the ratio and the thickness, the generation of holes and electric charges in the second hole transport layer may be promoted to facilitate the injection of holes into the second emission layer, thereby providing an organic electronic device superior in terms of lifespan or efficiency. It is possible to prevent the problem of short circuits in the device and prevent fabrication cost from being increased by excessive use of the doping material.

The second doping material-doped layer may include the second compound, and include 5 to 15 parts by weight of the second doping material with respect to 100 parts by weight of the second compound.

The second doping material-doped layer may include the second compound, and include 5 to 15 parts by weight of the second doping material with respect to 100 parts by weight of the second compound. The lower limit of the doping ratio of the second doping material may be, for example, 7 parts by weight or more or 9 parts by weight or more. The upper limit of the doping ratio of the second doping material may be, for example, 13 parts by weight or less or 11 parts by weight or less. The lower limit of the doping ratio of the second doping material may be, for example, 7 parts by weight or more or 9 parts by weight or more. The upper limit of the doping ratio of the second doping material may be, for example, 13 parts by weight or less or 11 parts by weight or less.

When the doping ratio of the second doping material meets the above-described range, the generation of holes and electric charges in the second hole transport layer may be promoted to facilitate the injection of holes into the second emission layer, thereby providing an organic electronic device superior in terms of lifespan or efficiency. It is possible to prevent the problem of short circuits in the device and prevent fabrication cost from being increased by excessive use of the doping material.

The third stack 143 may include a third hole transport region, a third emission layer, and a third electron transport region. Regarding the third hole transport region, the third emission layer, and the third electron transport region, what has been described above for the first hole transport region 1411, the first emission layer 1412, and the first electron transport region 1413 may equally be applied, unless clearly stated otherwise.

The third hole transport region may include a third hole transport layer and a third auxiliary emission layer. Regarding the third hole transport layer and the third auxiliary emission layer, what has been described above for the first hole transport layer 1411a and the first auxiliary emission layer 1411b may equally be applied, unless clearly stated otherwise.

Regarding the thickness and the doping of the third hole transport layer, what has been described above for the thickness and the doping of the first hole transport layer 1411a may equally be applied.

The thickness of the third hole transport layer may be from 250 Å to 700 Å. The lower limit of the thickness of the third hole transport layer may be, for example, 260 Å or more or 270 Å or more. The upper limit of the thickness of the third hole transport layer may be, for example, 650 Å or less or 600 Å or less.

When the thickness of the third hole transport layer meets the above-described range, the third hole transport layer may include a hole transport material in an amount sufficient to have superior hole injection and transport functions and prevent electric charges from being excessively injected, thereby providing an organic electronic device having a low thickness while being superior in terms of driving voltage, efficiency, or lifespan.

In the third hole transport layer, 10% to 50% of the thickness of the third hole transport layer may be doped with a third doping material. The portion of the third hole transport layer doped with the third doping material may be referred to as a third doping material-doped layer. The third hole transport layer may include the third doping material-doped layer doped with the third doping material and a third doping material undoped layer not doped with the third doping material. The third doping material undoped layer may be positioned between the third doping material-doped layer and the third emission layer.

For example, the third hole transport layer may include a hole transport material. The third doping material-doped layer may be a layer including the third doping material in addition to the hole transport material. The transport material is not particularly limited as long as it is a material having hole transport properties. For example, the hole transport material may be at least one of the first compound or the fourth compound.

The thickness of the third doping material-doped layer may be 10% to 50% of the thickness of the third hole transport layer. The lower limit of the ratio of the thickness of the third doping material-doped layer with respect to the thickness of the third hole transport layer may be, for example, 12% or more or 15% or more. The upper limit of the ratio of the thickness of the third doping material-doped layer with respect to the thickness of the third hole transport layer may be, for example, 40% or less or 30% or less.

The thickness of the third doping material-doped layer may be, for example, from 30 Å to 300 Å while meeting the above-described range of the ratio with respect to the thickness of the third hole transport layer. The lower limit of the thickness of the third doping material-doped layer may be, for example, 60 Å or more or 80 Å or more, whereas the upper limit of the thickness of the third doping material-doped layer may be, for example, 200 Å or less or 150 Å or less.

When the thickness of the third doping material-doped layer meets the above-described ranges of the ratio and the thickness, the generation of holes and electric charges in the third hole transport layer may be promoted to facilitate the injection of holes into the third emission layer, thereby providing an organic electronic device superior in terms of lifespan or efficiency. It is possible to prevent the problem of short circuits in the device and prevent fabrication cost from being increased by excessive use of the doping material.

The third doping material-doped layer may include the third compound, and include 5 to 15 parts by weight of the third doping material with respect to 100 parts by weight of the third compound.

The third doping material-doped layer may include the third compound, and include 5 to 15 parts by weight of the third doping material with respect to 100 parts by weight of the third compound. The lower limit of the doping ratio of the third doping material may be, for example, 7 parts by weight or more or 9 parts by weight or more. The upper limit of the doping ratio of the third doping material may be, for example, 13 parts by weight or less or 11 parts by weight or less. The lower limit of the doping ratio of the third doping material may be, for example, 7 parts by weight or more or 9 parts by weight or more. The upper limit of the doping ratio of the third doping material may be, for example, 13 parts by weight or less or 11 parts by weight or less.

When the doping ratio of the third doping material meets the above-described range, the generation of holes and electric charges in the third hole transport layer may be promoted to facilitate the injection of holes into the third emission layer, thereby providing an organic electronic device superior in terms of lifespan or efficiency. It is possible to prevent the problem of short circuits in the device and prevent fabrication cost from being increased by excessive use of the doping material.

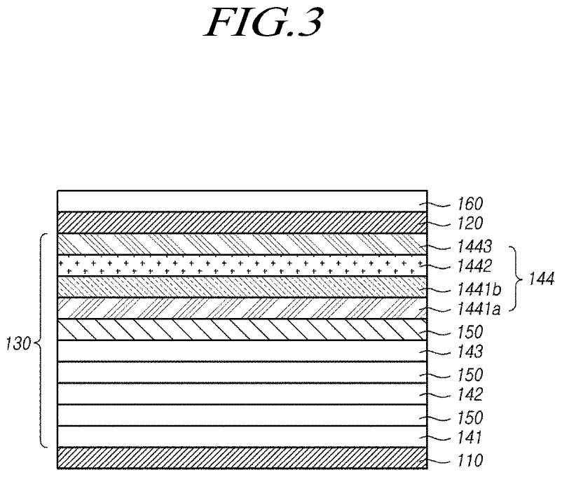

FIG. 3 is a diagram schematically illustrating an organic electronic device according to embodiments of the present disclosure.

Referring to FIG. 3, the organic material layer of the organic electronic device according to embodiments of the present disclosure may further include a fourth stack 144.

Regarding the fourth stack 144, the what has been described above for the first stack 141 may equally be applied, unless clearly stated otherwise.

In embodiments illustrated in FIG. 1, the first electrode 110, the first stack 141, the second stack 142, the third stack 143, the fourth stack 144, and the second electrode 120 are sequentially stacked. However, embodiments of the present disclosure are not limited to this organic electronic device. The upper and lower positional relationship of the first stack 141 to the fourth stack 144 may be different from that illustrated in FIG. 3 as long as the first stack 141 to the fourth stack 144 are positioned between the first electrode 110 and the second electrode 120.

The fourth stack 144 may include a fourth hole transport region 1441, a fourth emission layer 1442, and a fourth electron transport region 1443. Regarding the fourth hole transport region 1441, the fourth emission layer 1442, and fourth electron transport region, what has been described above for the first hole transport region 1411, the first emission layer 1412, and the first electron transport region 1413 may equally be applied, unless clearly stated otherwise.

The fourth hole transport region 1441 may include a fourth hole transport layer 1441a and a fourth auxiliary emission layer 1441b. Regarding the fourth hole transport layer 1441a and the fourth auxiliary emission layer 1441b, what has been described above for the first hole transport layer 1411a and the first auxiliary emission layer 1411b may equally be applied, unless clearly stated otherwise.

Regarding the thickness and the doping of the fourth hole transport layer 1441a, what has been described above for the thickness and the doping of the first hole transport layer 1411a may equally be applied, unless clearly stated otherwise.

The thickness of the fourth hole transport layer 1441a may be from 250 Å to 700 Å. The lower limit of the thickness of the fourth hole transport layer 1441a may be, for example, 260 Å or more or 270 Å or more. The upper limit of the thickness of the fourth hole transport layer 1441a may be, for example, 650 Å or less or 600 Å or less.

When the thickness of the fourth hole transport layer 1441a meets the above-described range, the fourth hole transport layer 1441a may include a hole transport material in an amount sufficient to have superior hole injection and transport functions and prevent electric charges from being excessively injected, thereby providing an organic electronic device having a low thickness while being superior in terms of driving voltage, efficiency, or lifespan.

In the fourth hole transport layer 1441a, 10% to 50% of the thickness of the fourth hole transport layer 1441a may be doped with a fourth doping material. The portion of the fourth hole transport layer 1441a doped with the fourth doping material may be referred to as a fourth doping material-doped layer. The fourth hole transport layer 1441a may include the fourth doping material-doped layer doped with the fourth doping material and a fourth doping material undoped layer not doped with the fourth doping material. The fourth doping material undoped layer may be positioned between the fourth doping material-doped layer and the fourth emission layer.

For example, the fourth hole transport layer may include a hole transport material. The fourth doping material-doped layer may be a layer including the fourth doping material in addition to the hole transport material. The transport material is not particularly limited as long as it is a material having hole transport properties. For example, the hole transport material may be at least one of a fifth compound or a sixth compound.

The thickness of the fourth doping material-doped layer may be 10% to 50% of the thickness of the fourth hole transport layer. The lower limit of the ratio of the thickness of the fourth doping material-doped layer with respect to the thickness of the fourth hole transport layer 1441a may be, for example, 12% or more or 15% or more. The upper limit of the ratio of the thickness of the fourth doping material-doped layer with respect to the thickness of the fourth hole transport layer 1441a may be, for example, 40% or less or 30% or less.

The thickness of the fourth doping material-doped layer may be, for example, from 30 Å to 300 Å while meeting the above-described range of the ratio with respect to the thickness of the fourth hole transport layer 1441a. The lower limit of the thickness of the fourth doping material-doped layer may be, for example, 60 Å or more or 80 Å or more, whereas the upper limit of the thickness of the fourth doping material-doped layer may be, for example, 200 Å or less or 150 Å or less.

When the thickness of the fourth doping material-doped layer meets the above-described ranges of the ratio and the thickness, the generation of holes and electric charges in the fourth hole transport layer 1441a may be promoted to facilitate the injection of holes into the fourth emission layer 1442, thereby providing an organic electronic device superior in terms of lifespan or efficiency. It is possible to prevent the problem of short circuits in the device and prevent fabrication cost from being increased by excessive use of the doping material.

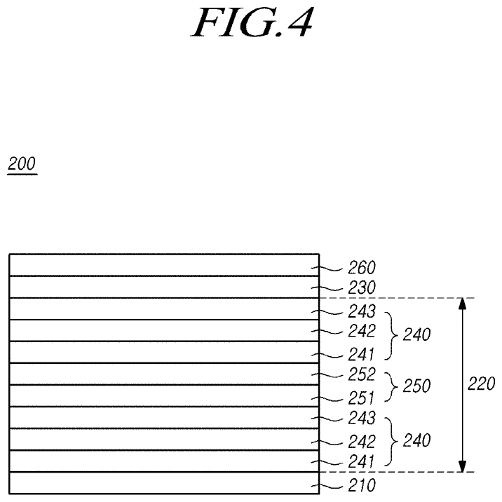

FIG. 4 is a diagram schematically illustrating an organic electronic device according to embodiments of the present disclosure.

An organic electronic device 200 according to embodiments includes a first electrode 210, a second electrode 230, and an organic material layer 220 positioned between the first electrode 210 and the second electrode 230 and including at least two stacks 240.

For example, the first electrode 210 may be an anode, whereas the second electrode 230 may be a cathode. The organic material layer 220 is a layer positioned between the first electrode 210 and the second electrode 230 and including an organic material. The organic material layer 220 may be comprised of a plurality of layers.

In an example, the first electrode 210 may be a transparent electrode, whereas the second electrode 230 may be a reflecting electrode. In another example, the first electrode 210 may be a reflecting electrode, whereas the second electrode 230 may be a transparent electrode.

Since the organic material layer 220 includes at least two stacks, the organic electronic device according to embodiments may be, for example, a tandem organic electronic device including a plurality of stacks. The organic material layer may be realized by repeatedly stacking the same stack two or more times or by stacking two or more different stacks.

Each of the above-described three or more stacks 240 includes a hole transport region 241, an emission layer 242, and an electron transport region 243. The hole transport region 241 may be a region, for example, positioned between the first electrode 210 serving as an anode and the emission layer 242 to transport holes from the first electrode 210 to the emission layer 242. The electron transport region 243 may be, for example, a region positioned between the second electrode 230 serving as a cathode and the emission layer 242 to transport electrons from the second electrode 230 to the emission layer.

The emission layer 242 is a layer in which energy generated by electron-hole recombination is emitted as light. For example, the emission layer 242 may include a host material and a dopant.

The thickness of the hole transport region may be from 10 nm to 100 nm. The lower limit of the thickness of the hole transport region may be, for example, 15 nm or more or 20 nm or more. The upper limit of the thickness of the hole transport region may be, for example, 80 nm or less or 60 nm or less. When the thickness of the hole transport region is within this range, the organic electronic device may have high luminous efficiency, a low driving voltage, and increased lifespan.

The organic material layer 220 may include one or more charge generation layers 250 positioned between the stacks 240. When three or more stacks 240 are provided, the charge generation layers 250 may be positioned between the stacks 240. Here, the plurality of charge generation layers 250 may be the same as or different from each other. Since the charge generation layers 250 are disposed between the stacks, the current efficiency of each of the stacks can be increased and electric charges can be properly distributed over the stacks.

The charge generation layers 250 may include, for example, an N-type charge generation layer 251 and a P-type charge generation layer 252. For example, the N-type charge generation layer 251 may be positioned adjacent to the first electrode 210 serving as an anode, whereas the P-type charge generation layer 252 may be positioned adjacent to the second electrode 230 serving as a cathode.

The charge generation layers 250 and the stacks 240 may be repeatedly positioned n times, where n is a positive integer. For example, n may be an integer from 1 to 5. For example, when n is 2, the organic material layer may include three stacks and two charge generation layers.

A capping layer 260 may be positioned above the second electrode 230. When the capping layer 260 is formed, the optical efficiency of the organic electronic device may be improved.

In a top emission organic electronic device, the capping layer 260 may serve to reduce optical energy loss in the second electrode 230 caused by SPPs. In a bottom emission organic electronic device, the capping layer 260 may serve to buffer the second electrode 230.

In the hole transport region 241 of each of the plurality of stacks 240, at least one hole transport region 241 includes a first hole transport layer and a second hole transport layer. For example, when the organic material layer includes two stacks, one of two hole transport layers of the two stacks may include a first hole transport layer and a second hole transport layer, or each of the two hole transport layers may include a first hole transport layer and a second hole transport layer.

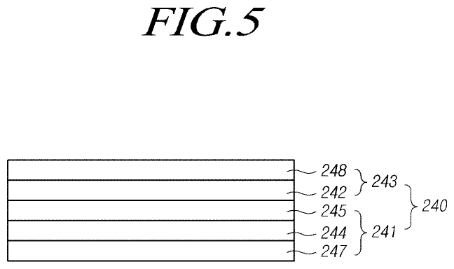

FIG. 5 is a diagram schematically illustrating a stack 240 according to embodiments of the present disclosure.

Referring to FIG. 5, at least one hole transport region 241 may include a first hole transport layer 244 and a second hole transport layer 245. For example, the first hole transport layer 244 and the second hole transport layer 245 may be positioned in the hole transport region 241 such that the first hole transport layer 244 is positioned more adjacent to the first electrode 210 than the second hole transport layer 245 and the second hole transport layer 245 is positioned more adjacent to the second electrode 230 than the first hole transport layer 244. In addition, the second hole transport layer 245 may be positioned more adjacent to the emission layer 242 than the first hole transport layer 244.

Although not shown in FIG. 5, the hole transport region 241 may further include a third hole transport layer (not shown). The third hole transport layer may be positioned, for example, between the first hole transport layer 244 and the second hole transport layer 245. In the above illustration, the first hole transport layer 244 may be positioned more adjacent to the first electrode 210 than the second hole transport layer 245, the second hole transport layer 245 may be positioned more adjacent to the emission layer 242 than the first hole transport layer 244, and the third hole transport layer may be positioned between the first hole transport layer 244 and the second hole transport layer 245.

The electron transport region 243 may include an electron transport layer 248.

The above-described first hole transport layer may include a seventh compound. The seventh compound may include a radical of a compound represented by Formula A to be described below, and may be represented by Formula C or Formula D to be described below. The above-described second hole transport layer may include an eighth compound. The eighth compound may include a radical of a compound represented by Formula A or Formula B to be described below, and may be represented by Formula C or Formula D to be described below.

As the first hole transport layer includes the seventh compound and the second hole transport layer includes the eighth compound, the luminous efficiency, lifespan, driving voltage, and color purity of the organic electronic device may be further improved.

The above-described third hole transport layer may include a ninth compound. The above-described ninth compound includes a radical of a compound represented by Formula A or Formula B to be described below, and is represented by Formula C or Formula D to be described below. In addition, the ninth compound is different from the eighth compound. When the hole transport region 241 further includes the above-described third hole transport layer, the luminous efficiency, lifespan, driving voltage, and color purity of the organic electronic device may be further improved.

The first hole transport layer including the seventh compound may mean that one or more types of the seventh compound are included. For example, the first hole transport layer may include two different types of seventh compound.

The second hole transport layer including the eighth compound may mean that one or more types of eighth compound are included. For example, the second hole transport layer may include two different types of eighth compound.

The third hole transport layer including the ninth compound may mean that one or more types of ninth compound are included. For example, the third hole transport layer may include two different types of ninth compound.

The organic electronic device according to embodiments of the present disclosure may be a top emission organic electronic device, a bottom emission organic electronic device, or a dual emission organic electronic device depending on the material used.

A white organic light emitting device (WOLED) is advantageous in that high resolution may be easily realized and processability is superior. In addition, the WOLED may be fabricated using conventional color filter technologies of liquid crystal displays (LCDs). A variety of structures have been proposed and patented for a white organic electronic device mainly used as a backlight unit. Representatively, there are a side-by-side method in which red (R), green (G), and blue (B) emission units are disposed in a planar direction, a stacking method in which R, G, and B emission layers are stacked in the top-bottom direction, a color conversion material (CCM) method using photoluminescence of an inorganic fluorescence material using electroluminescence caused by the blue (B) organic emission layer and light from the electroluminescence, and the like. The present disclosure may also be applied to such WOLEDs.

The first hole transport layer 1411a or the first auxiliary emission layer 1411b may include the first compound represented by the following Formula 1. In another example, the first hole transport layer 1411a and the first auxiliary emission layer 1411b may include the first compound represented by the following Formula 1.

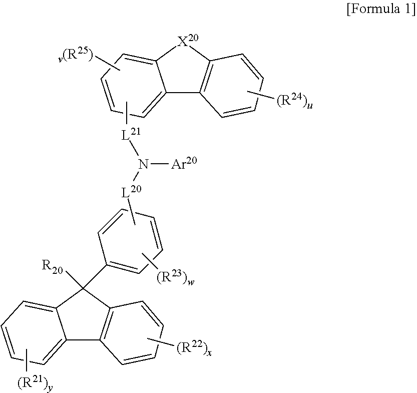

Hereinafter, Formula 1 will be described.

i) Each of R²⁰ to R²⁵ may be independently selected from the group consisting of deuterium; a halogen; a C₆-C₃₀ aryl group; a fluorenyl group; a C₂-C₃₀ heterocyclic group including at least one heteroatom of O, N, S, Si, or P; a fused ring group of a C₃-C₃₀ aliphatic ring and a C₆-C₃₀ aromatic ring; a C₁-C₃₀ alkyl group; a C₂-C₂₀ alkenyl group; a C₂-C₂₀ alkynyl group; a C₁-C₃₀ alkoxyl group; or a C₆-C₃₀ aryloxy group. Alternatively, ii) a plurality of R²¹s, a plurality of R²²s, a plurality of R²³s, a plurality of R²⁴s, and a plurality of R²⁵s may be bonded to form rings, respectively.

i) R²⁰ to R²⁵ may be respectively or independently selected from the group consisting of deuterium; a C₆-C₃₀ aryl group; a fluorenyl group; a C₂-C₃₀ heterocyclic group including at least one heteroatom of O, N, S, Si, or P; or a C₁-C₃₀ alkyl group. Alternatively, ii) a plurality of R²¹s, a plurality of R²²s, a plurality of R²³s, a plurality of R²⁴s, and a plurality of R²⁵s may be bonded to form rings, respectively.

When one of R²⁰ to R²⁵ is an aryl group, one of R²⁰ to R²⁵ which is an aryl group may be, for example, a C₆-C₆₀ aryl group, a C₆-C₄₀ aryl group, a C₆-C₂₅ aryl group, or a C₆-C₁₀ aryl group.

When one of R²⁰ to R²⁵ is a heterocyclic group, one of R²⁰ to R²⁵ which is a heterocyclic group may be, for example, a C₂-C₄₀ heterocyclic group including at least one hetero atom selected from O, N, S, Si, or P, a C₂-C₂₀ heterocyclic group including at least one hetero atom selected from O, N, S, Si, or P, or a C₂-C₁₀ heterocyclic group including at least one hetero atom selected from O, N, S, Si, or P.

When one of R²⁰ to R²⁵ is an alkyl group, one of R²⁰ to R²⁵ which is an alkyl group may be, for example, a C₁-C₃₀ alkyl group, a C₁-C₂₀ alkyl group, or a C₁-C₁₀ alkyl group.

When a plurality of R²¹s, a plurality of R²²s, a plurality of R²³s, a plurality of R²⁴s, and a plurality of R²⁵s are bonded to form rings, respectively, a benzene ring or a naphthalene ring, for example, may be formed.

v is an integer of one of 0 to 3.

Each of u, w, x, and y is independently an integer of one of 0 to 4.

Each of L²⁰ and L²¹ is independently selected from the group consisting of a single bond; a fluorenylene group; a C₆-C₃₀ arylene group; or a C₃-C₃₀ heterocyclic group including at least one hetero atom of O, N, S, Si, or P.

When one of L²⁰ or L²¹ is an aryl group, one of L²⁰ or L²¹ which is an aryl group may be, for example, a C₆-C₆₀ aryl group, a C₆-C₄₀ aryl group, a C₆-C₂₅ aryl group, or a C₆-C₁₀ aryl group.

When one of L²⁰ or L²¹ is a heterocyclic group, one of L²⁰ or L²¹ which is a heterocyclic group may be, for example, a C₂-C₄₀ heterocyclic group including at least one hetero atom of O, N, S, Si, or P, a C₂-C₂₀ heterocyclic group including at least one hetero atom of O, N, S, Si, or P, or a C₂-C₁₀ heterocyclic group including at least one hetero atom of O, N, S, Si, or P.

Ar²⁰ is a C₆-C₃₀ aryl group or a C₃-C₃₀ heterocyclic group including at least one hetero atom of O, N, S, Si, or P.

When Ar²⁰ is an aryl group, Ar²⁰ may be, for example, a C₆-C₆₀ aryl group, a C₆-C₄₀ aryl group, a C₆-C₂₅ aryl group, or a C₆-C₁₀ aryl group.

When Ar²⁰ is a heterocyclic group, Ar²⁰ may be, for example, a C₂-C₄₀ heterocyclic group including at least one hetero atom of O, N, S, Si, or P, a C₂-C₂₀ heterocyclic group including at least one hetero atom of O, N, S, Si, or P, or a C₂-C₁₀ heterocyclic group including at least one hetero atom of O, N, S, Si, or P.

X²⁰ is O, S, NR′, or CR′ R″.

R′ and R″ may be i) respectively and independently selected from the group consisting of a C₁-C₃₀ alkyl group; a C₆-C₃₀ aryl group; or a C₃-C₃₀ heterocyclic group including at least one hetero atom of O, N, S, Si, or P, or ii) bonded to form spiro compounds, respectively.

When one of R′ and R″ is an aryl group, one of R′ and R″ which is an aryl group may be, for example, a C₆-C₆₀ aryl group, a C₆-C₄₀ aryl group, a C₆-C₂₅ aryl group, or a C₆-C₁₀ aryl group.

When one of R′ and R″ is a heterocyclic group, one of R′ and R″ which is a heterocyclic group may be, for example, a C₂-C₄₀ heterocyclic group including at least one hetero atom of O, N, S, Si, or P, a C₂-C₂₀ heterocyclic group including at least one hetero atom of O, N, S, Si, or P, a C₂-C₁₀ heterocyclic group including at least one hetero atom of O, N, S, Si, or P.

When one of R′ or R″ is an alkyl group, one of R′ or R″ which is an alkyl group may be, for example, a C₁-C₃₀ alkyl group, a C₁-C₂₀ alkyl group, or a C₁-C₁₀ alkyl group.

When R′ and R″ are bonded to form spiro compounds, respectively, spirobifluorene, for example, may be formed.

In Formula 1, each of an aryl group, a fluorenyl group, a heterocyclic group, a fused ring group, an alkyl group, an alkenyl group, an alkynyl group, an alkoxyl group, an aryloxy group, an arylene group, and a fluorenylene group may be further substituted with one or more substituents selected from the group consisting of deuterium; a halogen group; a C₁-C₂₀ alkoxyl group; a C₁-C₂₀ alkyl group; a C₂-C₂₀ alkenyl group; a C₂-C₂₀ alkynyl group; a C₆-C₂₅ aryl group; a C₆-C₂₅ aryl group substituted with deuterium; a fluorenyl group; a C₂-C₂₀ heterocyclic group; or a C₃-C₂₀ cycloalkyl group.

Each of the further substituted substituents may be further substituted with one or more substituents selected from the group consisting of deuterium; a halogen group; a C₁-C₂₀ alkoxyl group; a C₁-C₂₀ alkyl group; a C₂-C₂₀ alkenyl group; a C₂-C₂₀ alkynyl group; a C₆-C₂₅ aryl group; a C₆-C₂₅ aryl group substituted with deuterium; a fluorenyl group; a C₂-C₂₀ heterocyclic group; or a C₃-C₂₀ cycloalkyl group. These substituents may be bonded to form a ring.

The first compound may be represented by the following Formula 2.

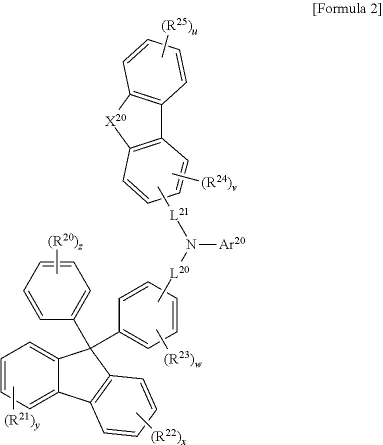

In Formula 2, z is an integer from 0 to 5, and u, v, w, x, y, R²⁰ to R²⁵, L²⁰, L²¹, Ar²⁰, and X²⁰ are the same as u, v, w, x, y, R²⁰ to R²⁵, L²⁰, L²¹, Ar²⁰, and X²⁰ defined in the description of Formula 1.

The first compound may be represented by one of the following Formula 3 to Formula 5.

[Formulas 3, 4, and 5]

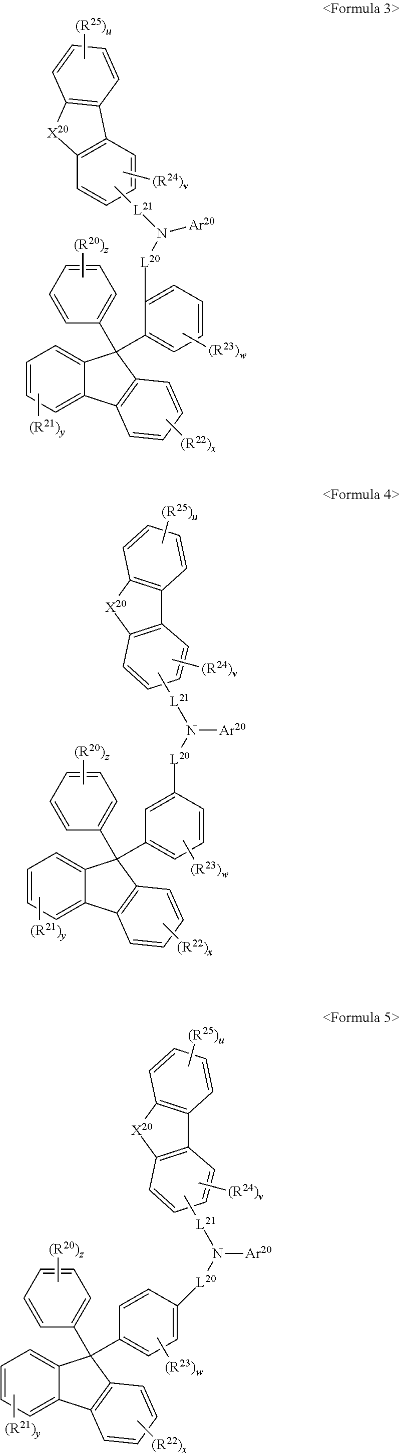

In Formula 3 to Formula 5, z is an integer from 0 to 5, and u, v, w, x, y, R²⁰ to R²⁵, L²⁰, L²¹, Ar²⁰, and X²⁰ are the same as u, v, w, x, y, R²⁰ to R²⁵, L²⁰, L²¹, Ar²⁰, and X²⁰ defined in the description of Formula 1.

The first compound may be represented by one of the following Formula 6 to Formula 9.

[Formulas 6, 7, 8, and 9]

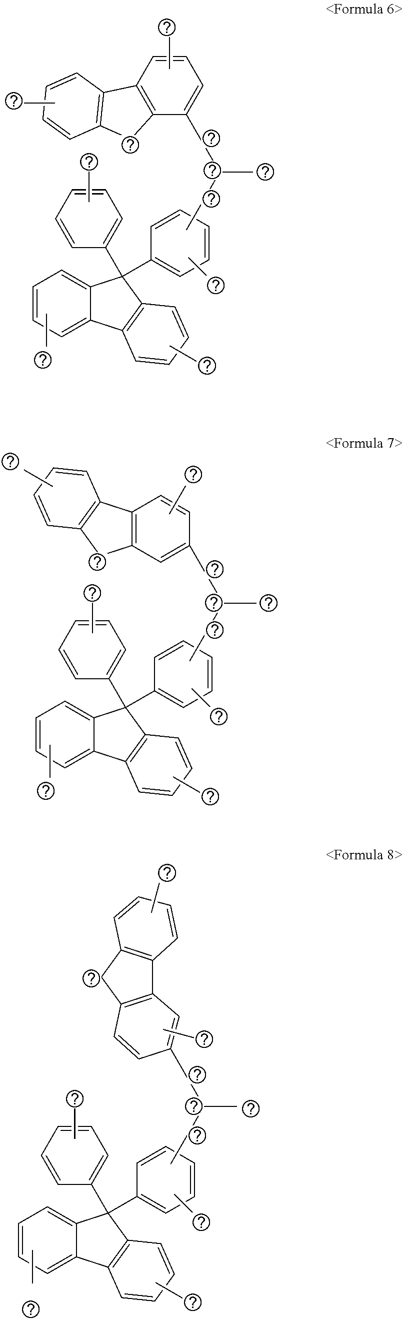

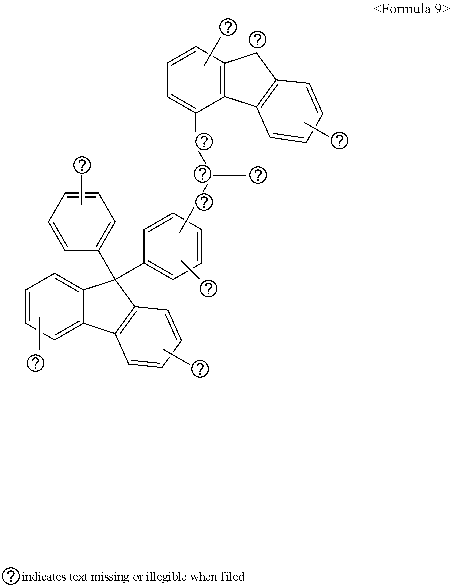

In Formula 6 to Formula 9, z is an integer from 0 to 5, and u, v, w, x, y, R²⁰ to R²⁵, L²⁰, L²¹, Ar²⁰, and X²⁰ are the same as u, v, w, x, y, R²⁰ to R²⁵, L²⁰, L²¹, Ar²⁰, and X²⁰ defined in the description of Formula 1.

The first compound may be represented by one of the following Formula 10 and Formula 11.

[Formulas 10 and 11]

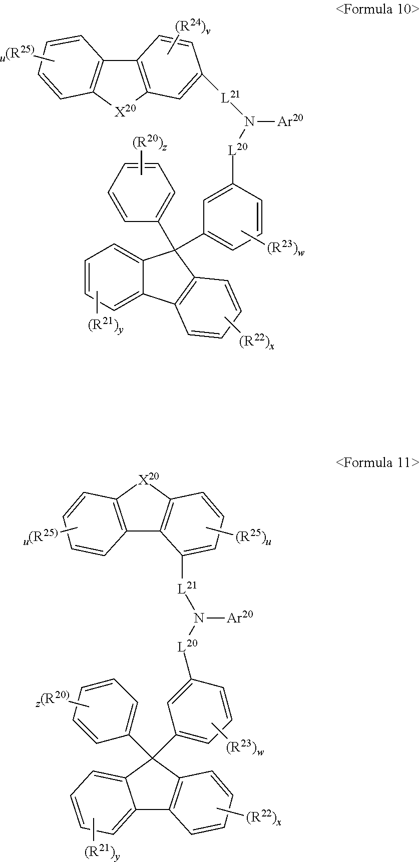

In Formula 10 to Formula 11, z is an integer from 0 to 5, and u, v, w, x, y, R²⁰ to R²⁵, L²⁰, L²¹, Ar²⁰, and X²⁰ are the same as u, v, w, x, y, R²⁰ to R²⁵, L²⁰, L²¹, Ar²⁰, and X²⁰ defined in the description of Formula 1.

The first compound may be represented by one of the following Formula 12 to Formula 13.

[Formulas 12 and 13]

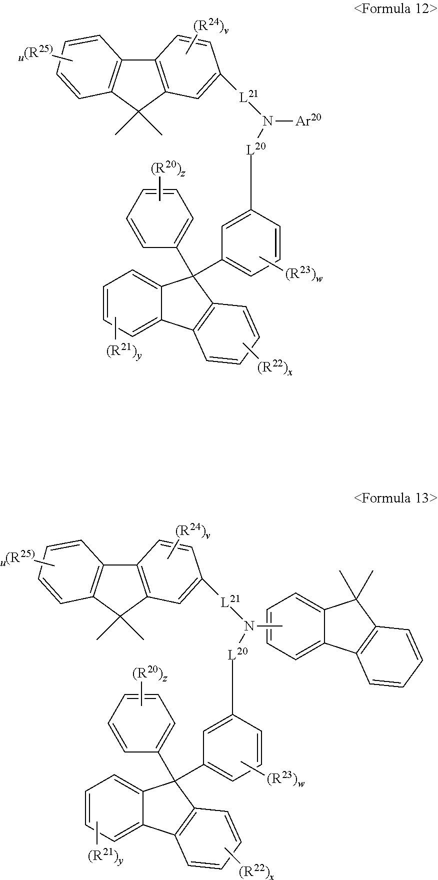

In Formula 12 to Formula 13, z is an integer from 0 to 5, and u, v, w, x, y, R²⁰ to R²⁵, L²⁰, L²¹, and Ar²⁰ are the same as u, v, w, x, y, R²⁰ to R²⁵, L²⁰, L²¹, and Ar²⁰ defined in the description of Formula 1.

The first compound may be one of the following compounds.

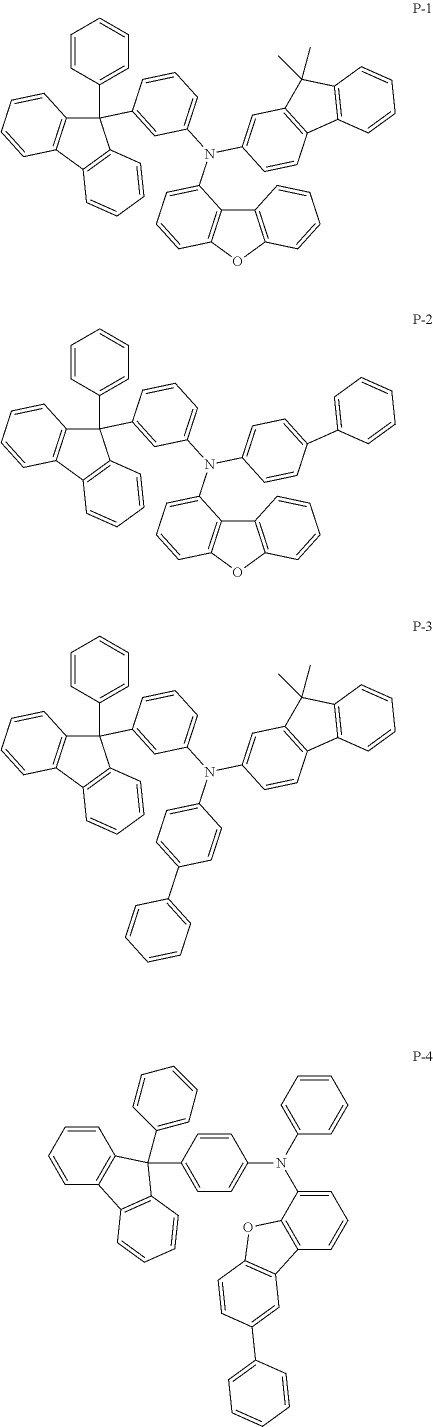 [SMILES: CC1(C)c2ccccc2-c2ccc(N(c3cccc(C4(c5ccccc5)c5ccccc5-c5ccccc54)c3)c3cccc4oc5ccccc5c34)cc21 ; c1ccc(-c2ccc(N(c3cccc(C4(c5ccccc5)c5ccccc5-c5ccccc54)c3)c3cccc4oc5ccccc5c34)cc2)cc1 ; CC1(C)c2ccccc2-c2ccc(N(c3ccc(-c4ccccc4)cc3)c3cccc(C4(c5ccccc5)c5ccccc5-c5ccccc54)c3)cc21 ; c1ccc(-c2ccc3oc4c(N(c5ccccc5)c5ccc(C6(c7ccccc7)c7ccccc7-c7ccccc76)cc5)cccc4c3c2)cc1]

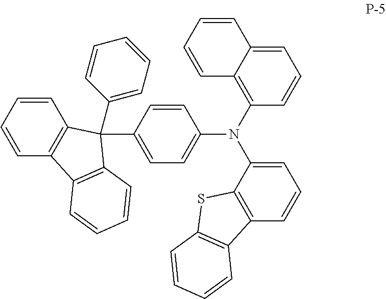 [SMILES: c1ccc(C2(c3ccc(N(c4cccc5ccccc45)c4cccc5c4sc4ccccc45)cc3)c3ccccc3-c3ccccc32)cc1]

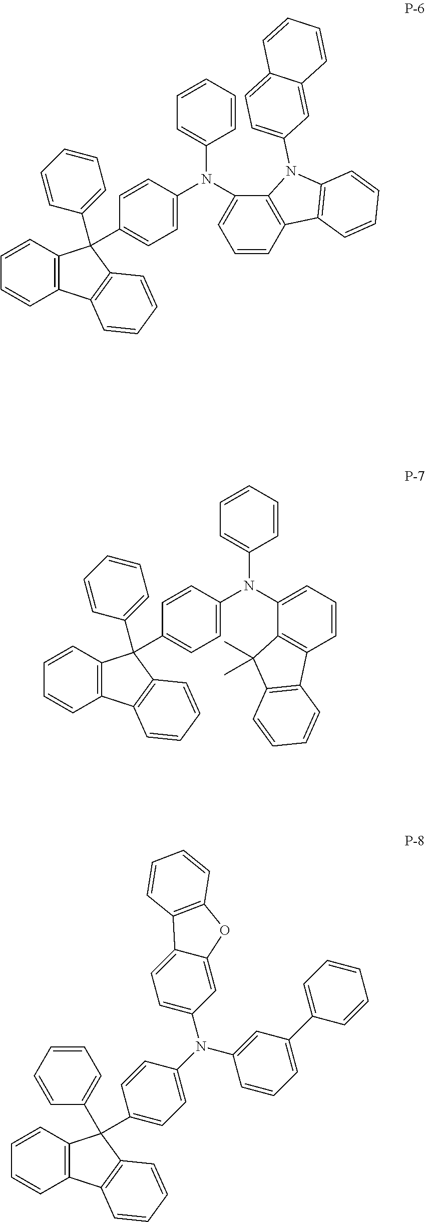 [SMILES: c1ccc(N(c2ccc(C3(c4ccccc4)c4ccccc4-c4ccccc43)cc2)c2cccc3c4ccccc4n(-c4ccc5ccccc5c4)c23)cc1 ; CC1(C)c2ccccc2-c2cccc(N(c3ccccc3)c3ccc(C4(c5ccccc5)c5ccccc5-c5ccccc54)cc3)c21 ; c1ccc(-c2cccc(N(c3ccc(C4(c5ccccc5)c5ccccc5-c5ccccc54)cc3)c3ccc4c(c3)oc3ccccc34)c2)cc1]

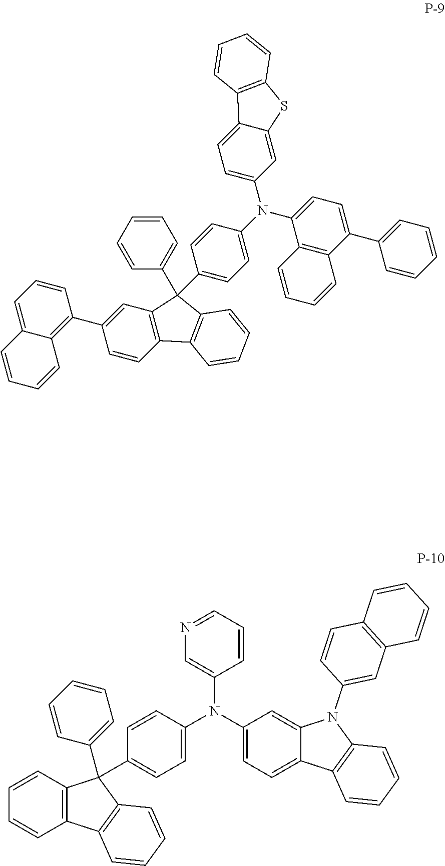 [SMILES: c1ccc(-c2ccc(N(c3ccc(C4(c5ccccc5)c5ccccc5-c5ccc(-c6cccc7ccccc67)cc54)cc3)c3ccc4c(c3)sc3ccccc34)c3ccccc23)cc1 ; c1ccc(C2(c3ccc(N(c4cccnc4)c4ccc5c6ccccc6n(-c6ccc7ccccc7c6)c5c4)cc3)c3ccccc3-c3ccccc32)cc1]

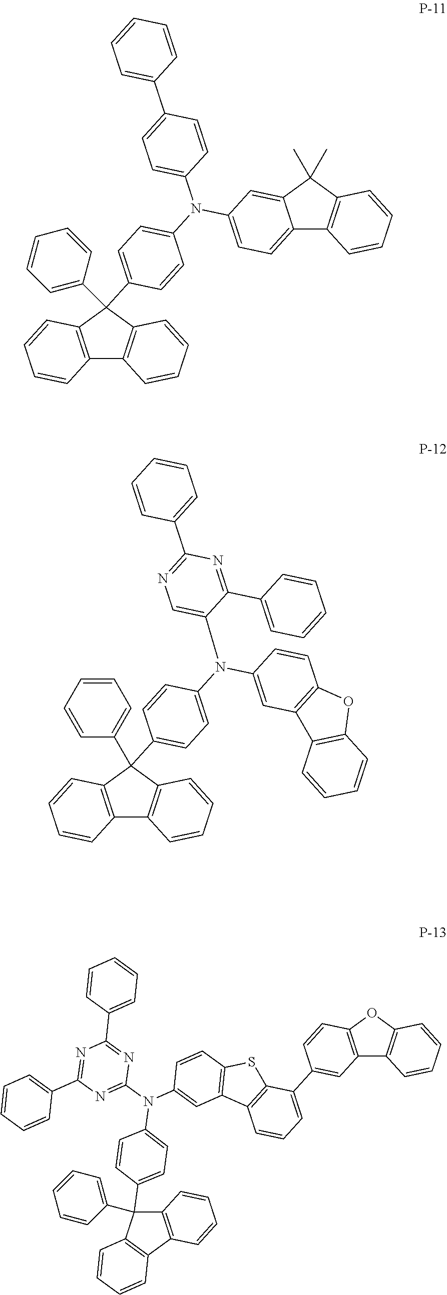 [SMILES: CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ncc(N(c3ccc(C4(c5ccccc5)c5ccccc5-c5ccccc54)cc3)c3ccc4oc5ccccc5c4c3)c(-c3ccccc3)n2)cc1 ; c1ccc(-c2nc(-c3ccccc3)nc(N(c3ccc(C4(c5ccccc5)c5ccccc5-c5ccccc54)cc3)c3ccc4sc5c(-c6ccc7oc8ccccc8c7c6)cccc5c4c3)n2)cc1]

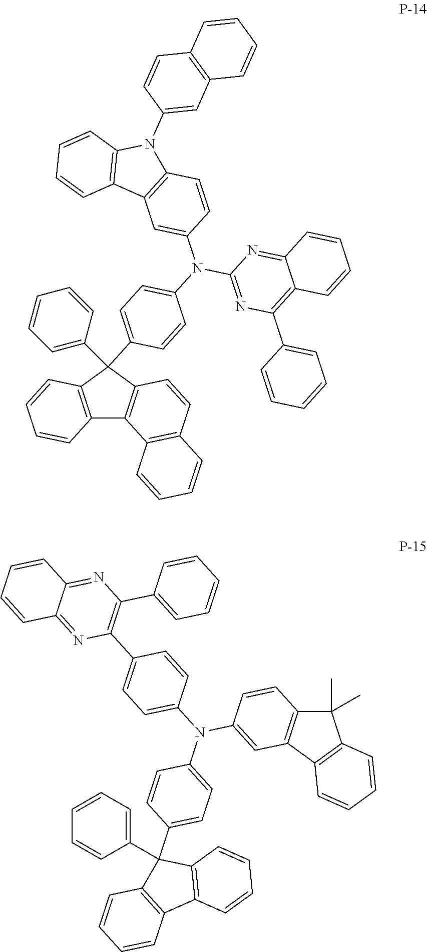 [SMILES: c1ccc(-c2nc(N(c3ccc(C4(c5ccccc5)c5ccccc5-c5c4ccc4ccccc54)cc3)c3ccc4c(c3)c3ccccc3n4-c3ccc4ccccc4c3)nc3ccccc23)cc1 ; CC1(C)c2ccccc2-c2cc(N(c3ccc(-c4nc5ccccc5nc4-c4ccccc4)cc3)c3ccc(C4(c5ccccc5)c5ccccc5-c5ccccc54)cc3)ccc21]

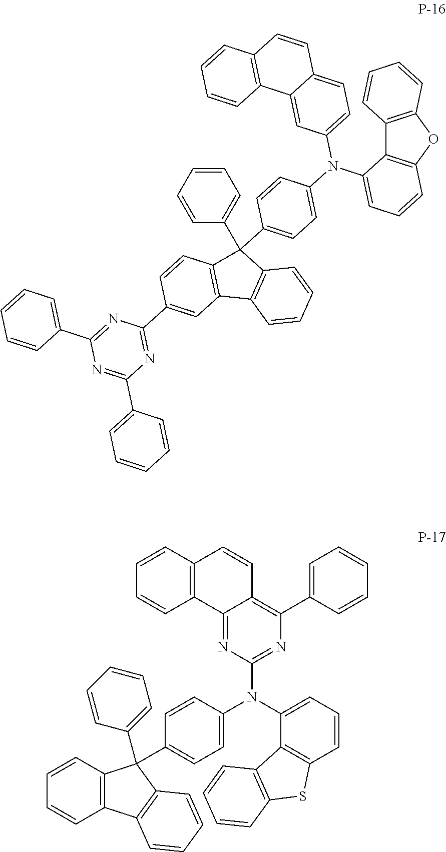 [SMILES: c1ccc(-c2nc(-c3ccccc3)nc(-c3ccc4c(c3)-c3ccccc3C4(c3ccccc3)c3ccc(N(c4ccc5ccc6ccccc6c5c4)c4cccc5oc6ccccc6c45)cc3)n2)cc1 ; c1ccc(-c2nc(N(c3ccc(C4(c5ccccc5)c5ccccc5-c5ccccc54)cc3)c3cccc4sc5ccccc5c34)nc3c2ccc2ccccc23)cc1]

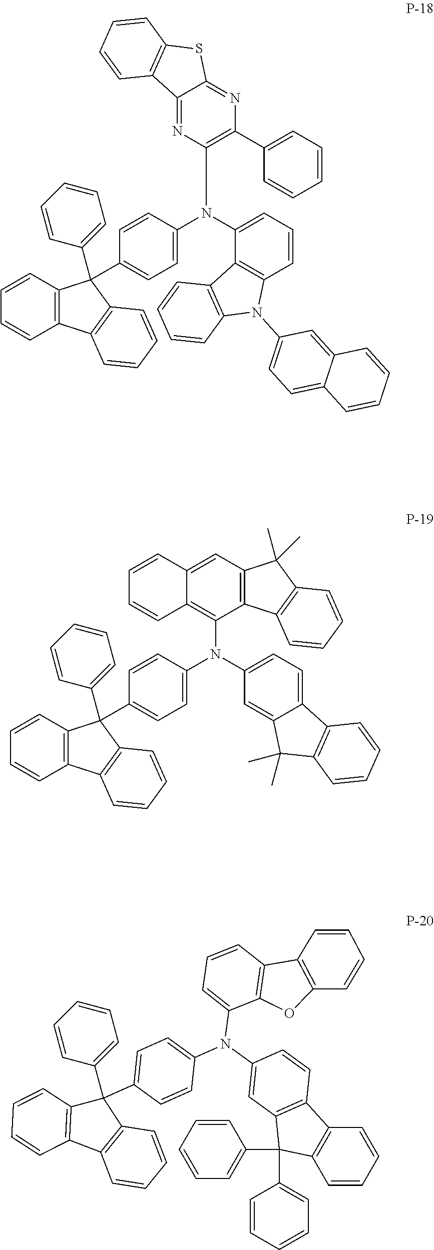 [SMILES: c1ccc(-c2nc3sc4ccccc4c3nc2N(c2ccc(C3(c4ccccc4)c4ccccc4-c4ccccc43)cc2)c2cccc3c2c2ccccc2n3-c2ccc3ccccc3c2)cc1 ; CC1(C)c2ccccc2-c2ccc(N(c3ccc(C4(c5ccccc5)c5ccccc5-c5ccccc54)cc3)c3c4c(cc5ccccc35)C(C)(C)c3ccccc3-4)cc21 ; c1ccc(C2(c3ccc(N(c4ccc5c(c4)C(c4ccccc4)(c4ccccc4)c4ccccc4-5)c4cccc5c4oc4ccccc45)cc3)c3ccccc3-c3ccccc32)cc1]

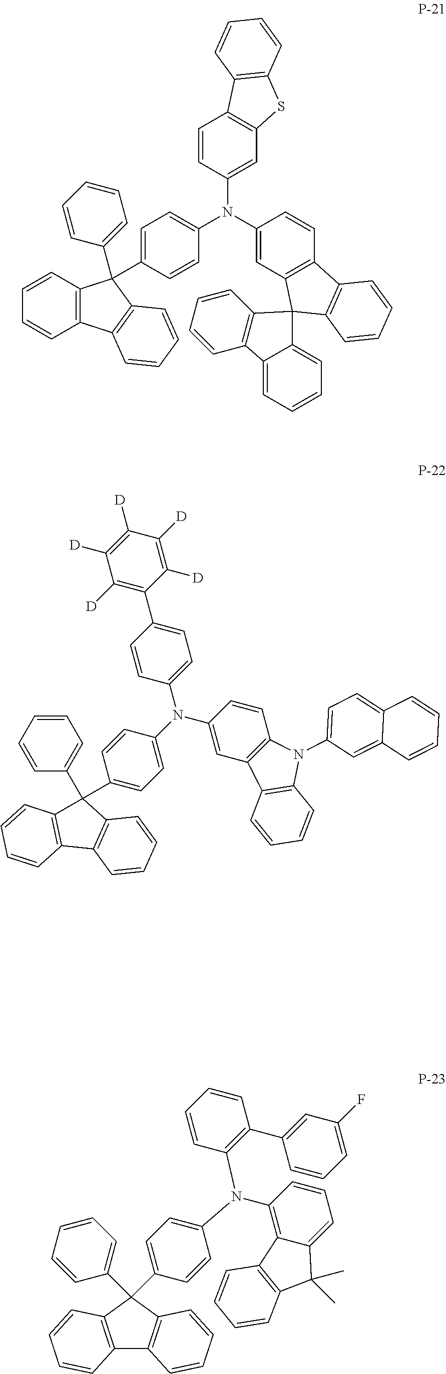 [SMILES: c1ccc(C2(c3ccc(N(c4ccc5c(c4)C4(c6ccccc6-c6ccccc64)c4ccccc4-5)c4ccc5c(c4)sc4ccccc45)cc3)c3ccccc3-c3ccccc32)cc1 ; [2H]c1c([2H])c([2H])c(-c2ccc(N(c3ccc(C4(c5ccccc5)c5ccccc5-c5ccccc54)cc3)c3ccc4c(c3)c3ccccc3n4-c3ccc4ccccc4c3)cc2)c([2H])c1[2H] ; CC1(C)c2ccccc2-c2c(N(c3ccc(C4(c5ccccc5)c5ccccc5-c5ccccc54)cc3)c3ccccc3-c3cccc(F)c3)cccc21]

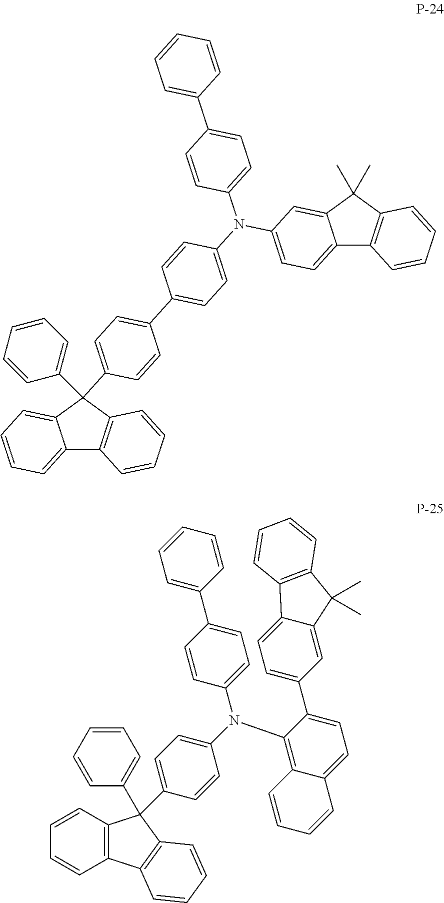 [SMILES: CC1(C)c2ccccc2-c2ccc(N(c3ccc(-c4ccccc4)cc3)c3ccc(-c4ccc(C5(c6ccccc6)c6ccccc6-c6ccccc65)cc4)cc3)cc21 ; CC1(C)c2ccccc2-c2ccc(-c3ccc4ccccc4c3N(c3ccc(-c4ccccc4)cc3)c3ccc(C4(c5ccccc5)c5ccccc5-c5ccccc54)cc3)cc21]

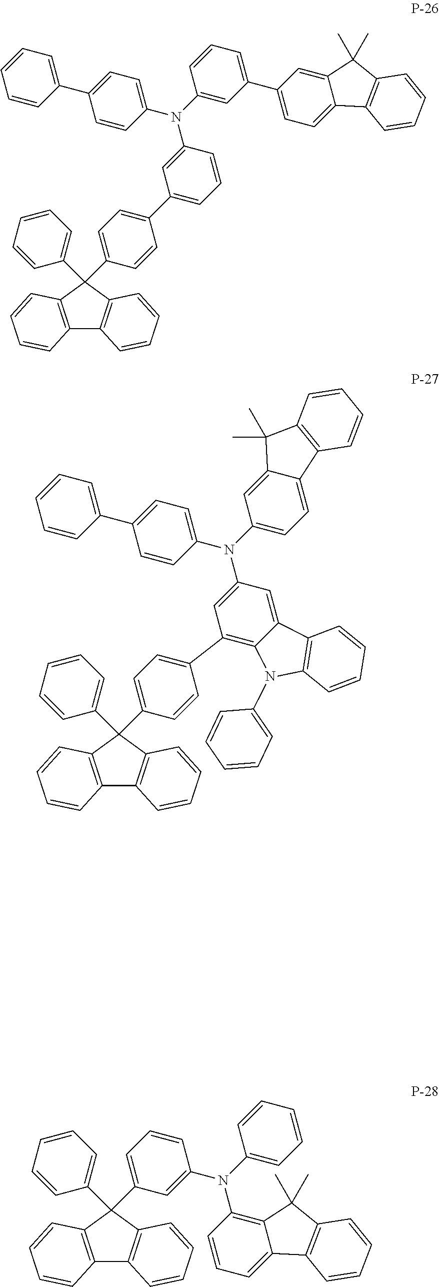 [SMILES: CC1(C)c2ccccc2-c2ccc(-c3cccc(N(c4ccc(-c5ccccc5)cc4)c4cccc(-c5ccc(C6(c7ccccc7)c7ccccc7-c7ccccc76)cc5)c4)c3)cc21 ; CC1(C)c2ccccc2-c2ccc(N(c3ccc(-c4ccccc4)cc3)c3cc(-c4ccc(C5(c6ccccc6)c6ccccc6-c6ccccc65)cc4)c4c(c3)c3ccccc3n4-c3ccccc3)cc21 ; CC1(C)c2ccccc2-c2cccc(N(c3ccccc3)c3cccc(C4(c5ccccc5)c5ccccc5-c5ccccc54)c3)c21]

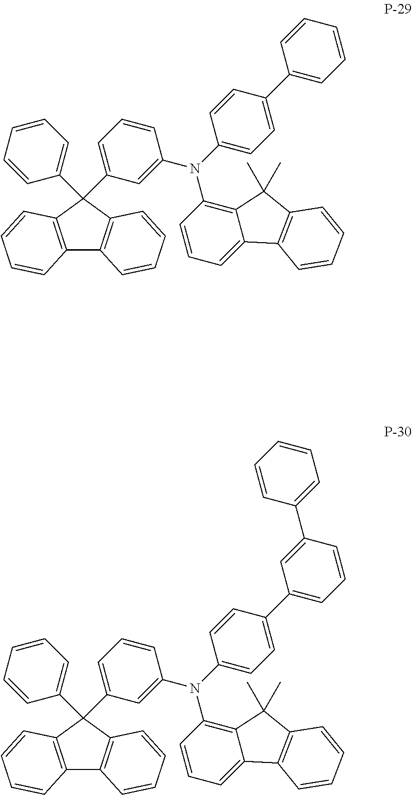 [SMILES: CC1(C)c2ccccc2-c2cccc(N(c3ccc(-c4ccccc4)cc3)c3cccc(C4(c5ccccc5)c5ccccc5-c5ccccc54)c3)c21 ; CC1(C)c2ccccc2-c2cccc(N(c3ccc(-c4cccc(-c5ccccc5)c4)cc3)c3cccc(C4(c5ccccc5)c5ccccc5-c5ccccc54)c3)c21]

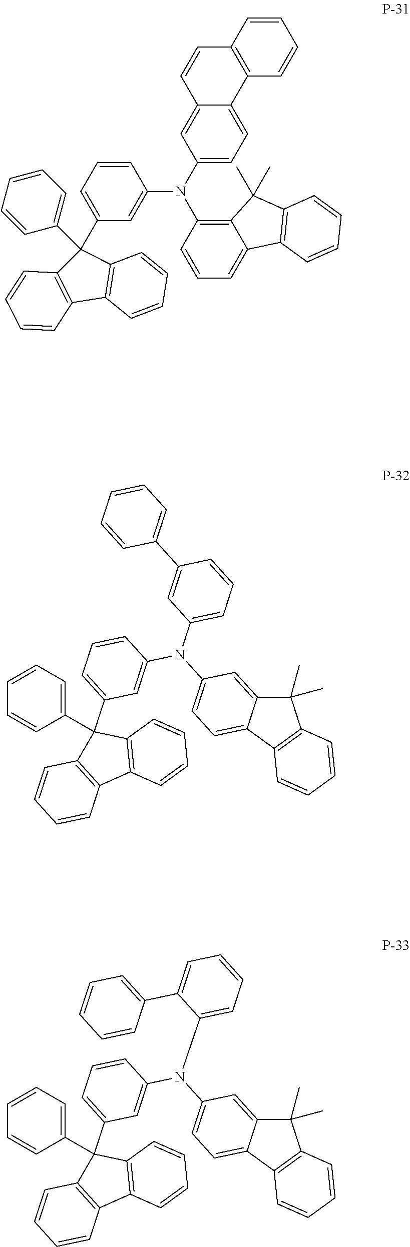 [SMILES: CC1(C)c2ccccc2-c2cccc(N(c3cccc(C4(c5ccccc5)c5ccccc5-c5ccccc54)c3)c3ccc4c(ccc5ccccc54)c3)c21 ; CC1(C)c2ccccc2-c2ccc(N(c3cccc(-c4ccccc4)c3)c3cccc(C4(c5ccccc5)c5ccccc5-c5ccccc54)c3)cc21 ; CC1(C)c2ccccc2-c2ccc(N(c3cccc(C4(c5ccccc5)c5ccccc5-c5ccccc54)c3)c3ccccc3-c3ccccc3)cc21]

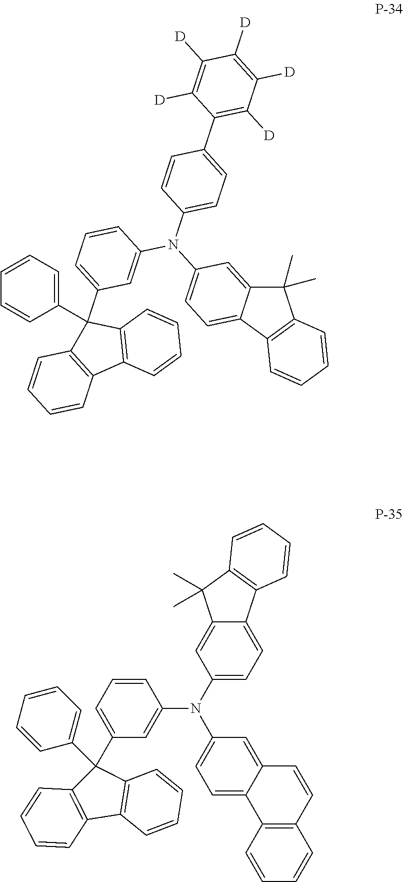 [SMILES: [2H]c1c([2H])c([2H])c(-c2ccc(N(c3cccc(C4(c5ccccc5)c5ccccc5-c5ccccc54)c3)c3ccc4c(c3)C(C)(C)c3ccccc3-4)cc2)c([2H])c1[2H] ; CC1(C)c2ccccc2-c2ccc(N(c3cccc(C4(c5ccccc5)c5ccccc5-c5ccccc54)c3)c3ccc4c(ccc5ccccc54)c3)cc21]

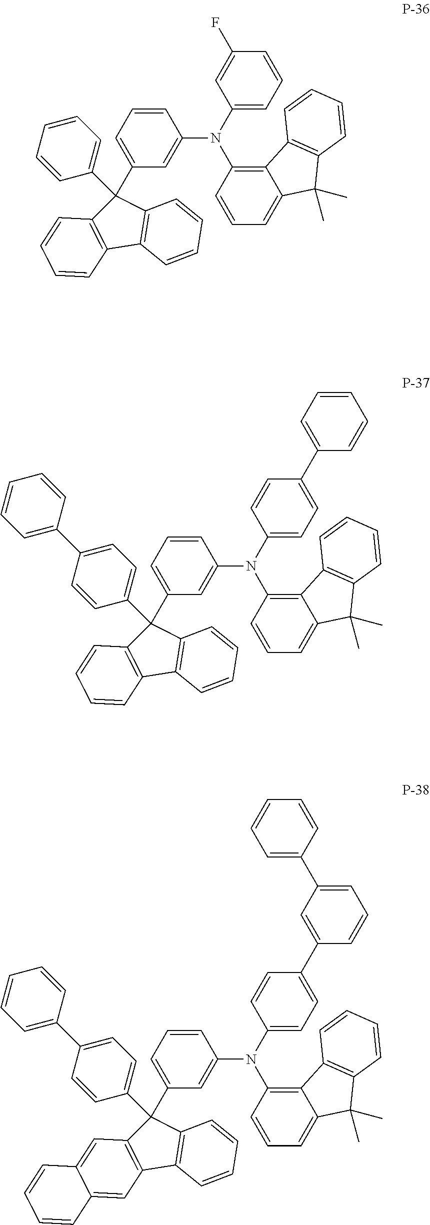 [SMILES: CC1(C)c2ccccc2-c2c(N(c3cccc(F)c3)c3cccc(C4(c5ccccc5)c5ccccc5-c5ccccc54)c3)cccc21 ; CC1(C)c2ccccc2-c2c(N(c3ccc(-c4ccccc4)cc3)c3cccc(C4(c5ccc(-c6ccccc6)cc5)c5ccccc5-c5ccccc54)c3)cccc21 ; CC1(C)c2ccccc2-c2c(N(c3ccc(-c4cccc(-c5ccccc5)c4)cc3)c3cccc(C4(c5ccc(-c6ccccc6)cc5)c5ccccc5-c5cc6ccccc6cc54)c3)cccc21]

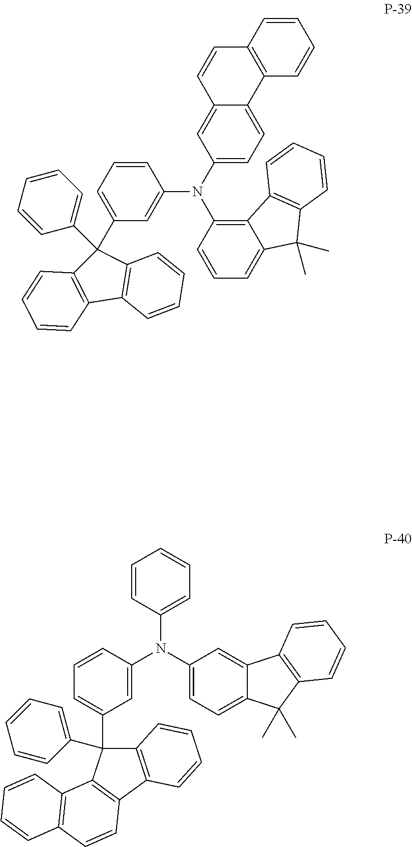 [SMILES: CC1(C)c2ccccc2-c2c(N(c3cccc(C4(c5ccccc5)c5ccccc5-c5ccccc54)c3)c3ccc4c(ccc5ccccc54)c3)cccc21 ; CC1(C)c2ccccc2-c2cc(N(c3ccccc3)c3cccc(C4(c5ccccc5)c5ccccc5-c5ccc6ccccc6c54)c3)ccc21]

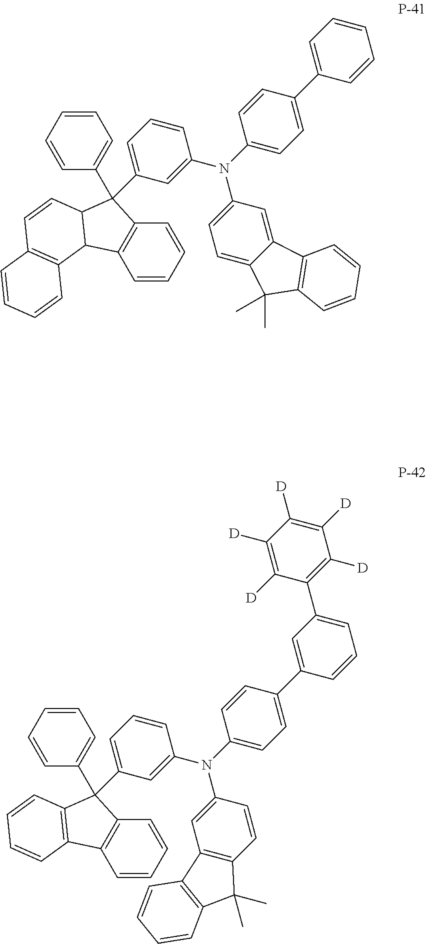 [SMILES: CC1(C)c2ccccc2-c2cc(N(c3ccc(-c4ccccc4)cc3)c3cccc(C4(c5ccccc5)c5ccccc5C5c6ccccc6C=CC54)c3)ccc21 ; [2H]c1c([2H])c([2H])c(-c2cccc(-c3ccc(N(c4cccc(C5(c6ccccc6)c6ccccc6-c6ccccc65)c4)c4ccc5c(c4)-c4ccccc4C5(C)C)cc3)c2)c([2H])c1[2H]]

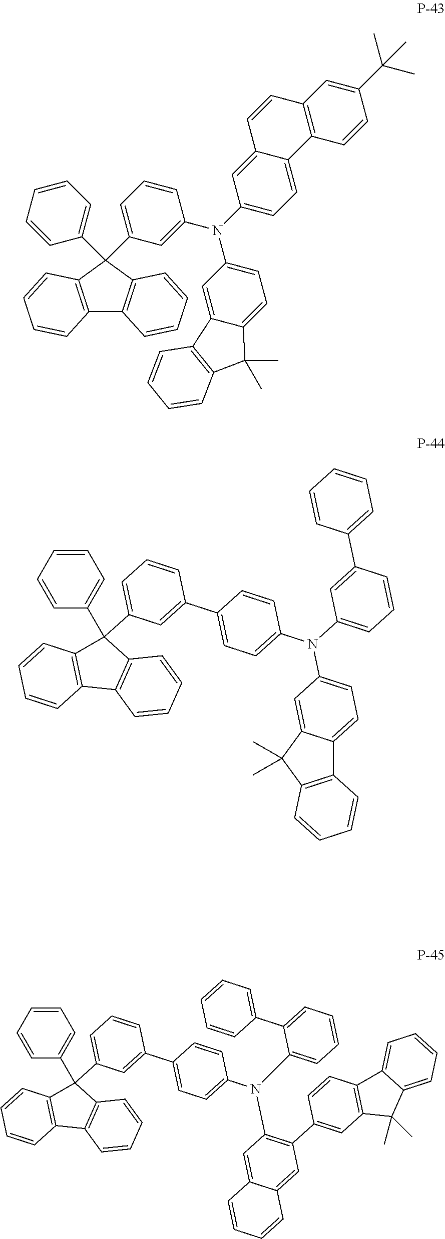 [SMILES: CC(C)(C)c1ccc2c(ccc3cc(N(c4cccc(C5(c6ccccc6)c6ccccc6-c6ccccc65)c4)c4ccc5c(c4)-c4ccccc4C5(C)C)ccc32)c1 ; CC1(C)c2ccccc2-c2ccc(N(c3ccc(-c4cccc(C5(c6ccccc6)c6ccccc6-c6ccccc65)c4)cc3)c3cccc(-c4ccccc4)c3)cc21 ; CC1(C)c2ccccc2-c2ccc(-c3cc4ccccc4cc3N(c3ccc(-c4cccc(C5(c6ccccc6)c6ccccc6-c6ccccc65)c4)cc3)c3ccccc3-c3ccccc3)cc21]

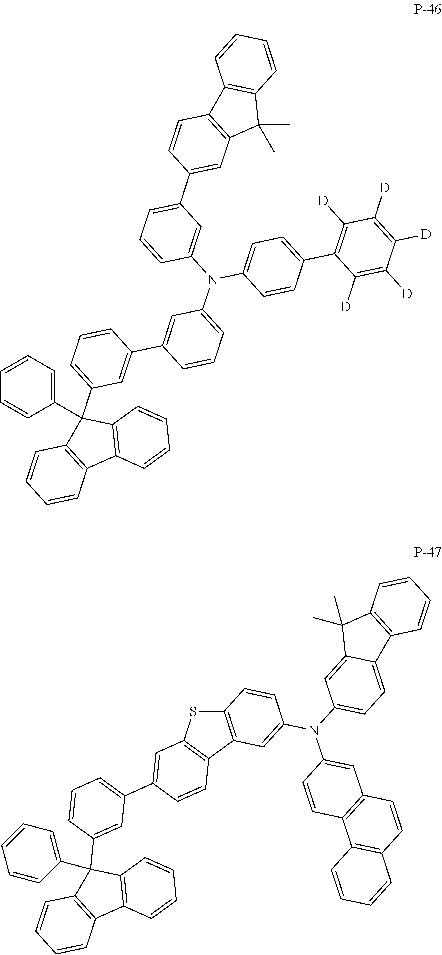 [SMILES: [2H]c1c([2H])c([2H])c(-c2ccc(N(c3cccc(-c4cccc(C5(c6ccccc6)c6ccccc6-c6ccccc65)c4)c3)c3cccc(-c4ccc5c(c4)C(C)(C)c4ccccc4-5)c3)cc2)c([2H])c1[2H] ; CC1(C)c2ccccc2-c2ccc(N(c3ccc4c(ccc5ccccc54)c3)c3ccc4sc5cc(-c6cccc(C7(c8ccccc8)c8ccccc8-c8ccccc87)c6)ccc5c4c3)cc21]

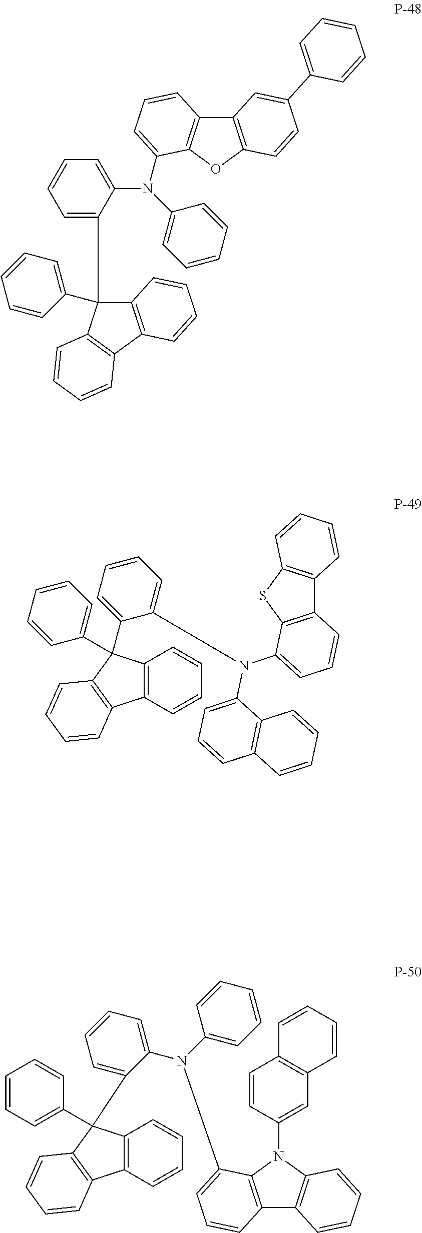 [SMILES: c1ccc(-c2ccc3oc4c(N(c5ccccc5)c5ccccc5C5(c6ccccc6)c6ccccc6-c6ccccc65)cccc4c3c2)cc1 ; c1ccc(C2(c3ccccc3N(c3cccc4ccccc34)c3cccc4c3sc3ccccc34)c3ccccc3-c3ccccc32)cc1 ; c1ccc(N(c2ccccc2C2(c3ccccc3)c3ccccc3-c3ccccc32)c2cccc3c4ccccc4n(-c4ccc5ccccc5c4)c23)cc1]

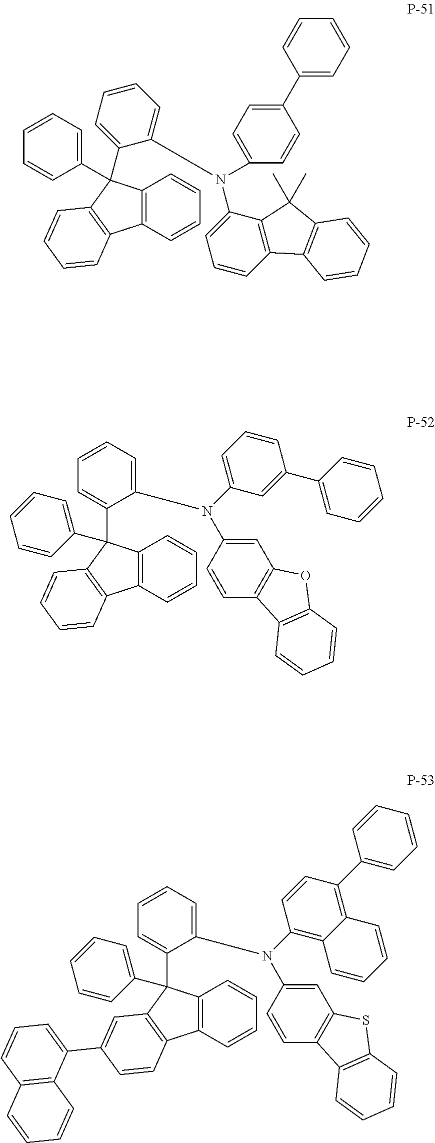 [SMILES: CC1(C)c2ccccc2-c2cccc(N(c3ccc(-c4ccccc4)cc3)c3ccccc3C3(c4ccccc4)c4ccccc4-c4ccccc43)c21 ; c1ccc(-c2cccc(N(c3ccc4c(c3)oc3ccccc34)c3ccccc3C3(c4ccccc4)c4ccccc4-c4ccccc43)c2)cc1 ; c1ccc(-c2ccc(N(c3ccc4c(c3)sc3ccccc34)c3ccccc3C3(c4ccccc4)c4ccccc4-c4ccc(-c5cccc6ccccc56)cc43)c3ccccc23)cc1]

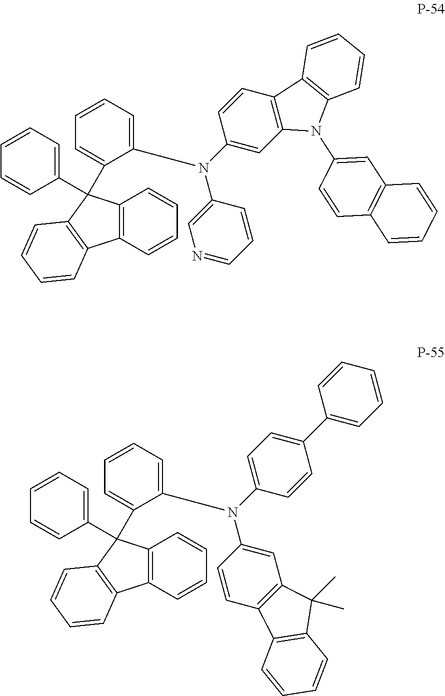 [SMILES: c1ccc(C2(c3ccccc3N(c3cccnc3)c3ccc4c5ccccc5n(-c5ccc6ccccc6c5)c4c3)c3ccccc3-c3ccccc32)cc1 ; CC1(C)c2ccccc2-c2ccc(N(c3ccc(-c4ccccc4)cc3)c3ccccc3C3(c4ccccc4)c4ccccc4-c4ccccc43)cc21]

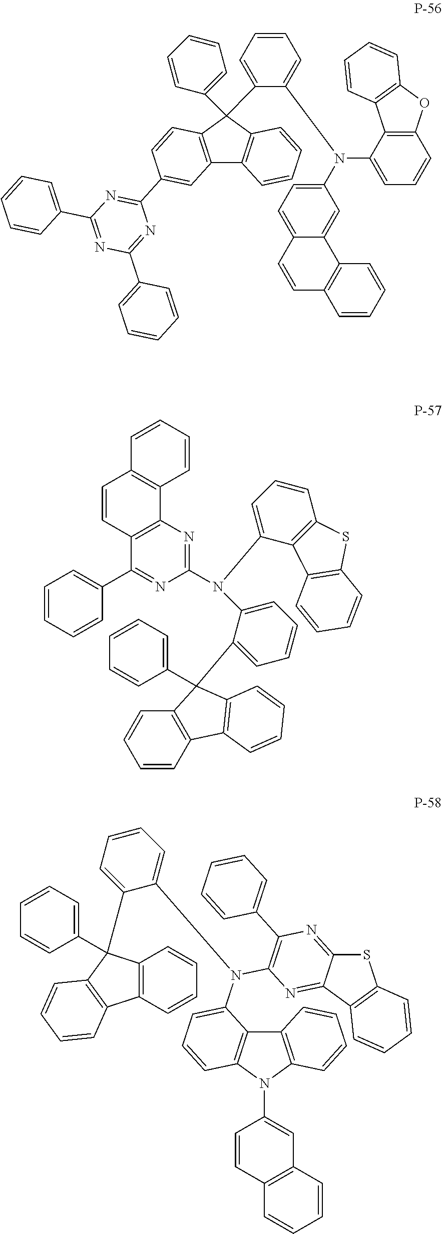 [SMILES: c1ccc(-c2nc(-c3ccccc3)nc(-c3ccc4c(c3)-c3ccccc3C4(c3ccccc3)c3ccccc3N(c3ccc4ccc5ccccc5c4c3)c3cccc4oc5ccccc5c34)n2)cc1 ; c1ccc(-c2nc(N(c3ccccc3C3(c4ccccc4)c4ccccc4-c4ccccc43)c3cccc4sc5ccccc5c34)nc3c2ccc2ccccc23)cc1 ; c1ccc(-c2nc3sc4ccccc4c3nc2N(c2ccccc2C2(c3ccccc3)c3ccccc3-c3ccccc32)c2cccc3c2c2ccccc2n3-c2ccc3ccccc3c2)cc1]

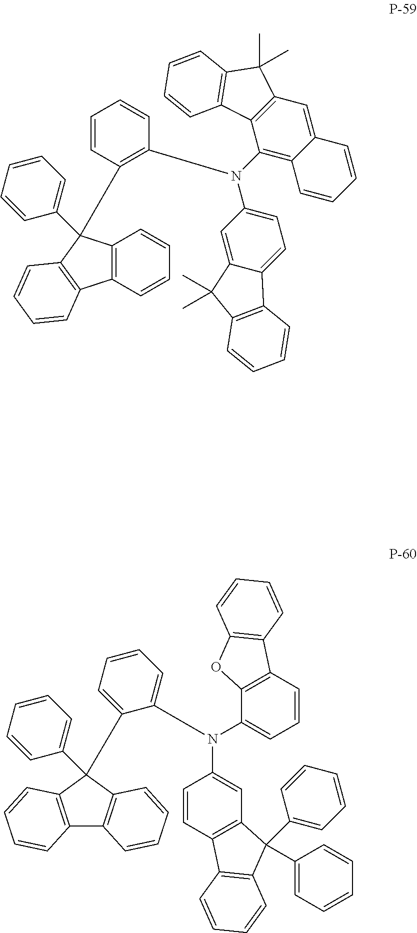 [SMILES: CC1(C)c2ccccc2-c2ccc(N(c3ccccc3C3(c4ccccc4)c4ccccc4-c4ccccc43)c3c4c(cc5ccccc35)C(C)(C)c3ccccc3-4)cc21 ; c1ccc(C2(c3ccccc3)c3ccccc3-c3ccc(N(c4ccccc4C4(c5ccccc5)c5ccccc5-c5ccccc54)c4cccc5c4oc4ccccc45)cc32)cc1]

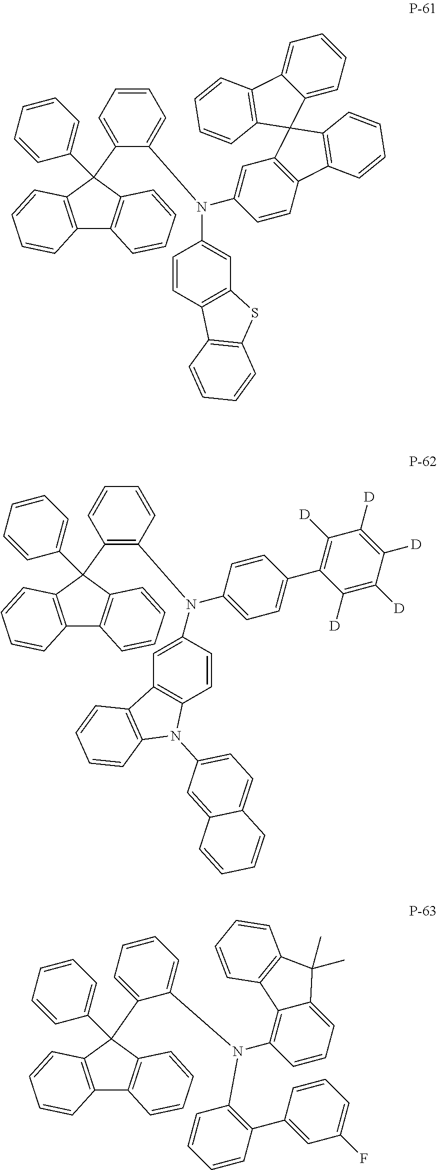 [SMILES: c1ccc(C2(c3ccccc3N(c3ccc4c(c3)C3(c5ccccc5-c5ccccc53)c3ccccc3-4)c3ccc4c(c3)sc3ccccc34)c3ccccc3-c3ccccc32)cc1 ; [2H]c1c([2H])c([2H])c(-c2ccc(N(c3ccc4c(c3)c3ccccc3n4-c3ccc4ccccc4c3)c3ccccc3C3(c4ccccc4)c4ccccc4-c4ccccc43)cc2)c([2H])c1[2H] ; CC1(C)c2ccccc2-c2c(N(c3ccccc3-c3cccc(F)c3)c3ccccc3C3(c4ccccc4)c4ccccc4-c4ccccc43)cccc21]

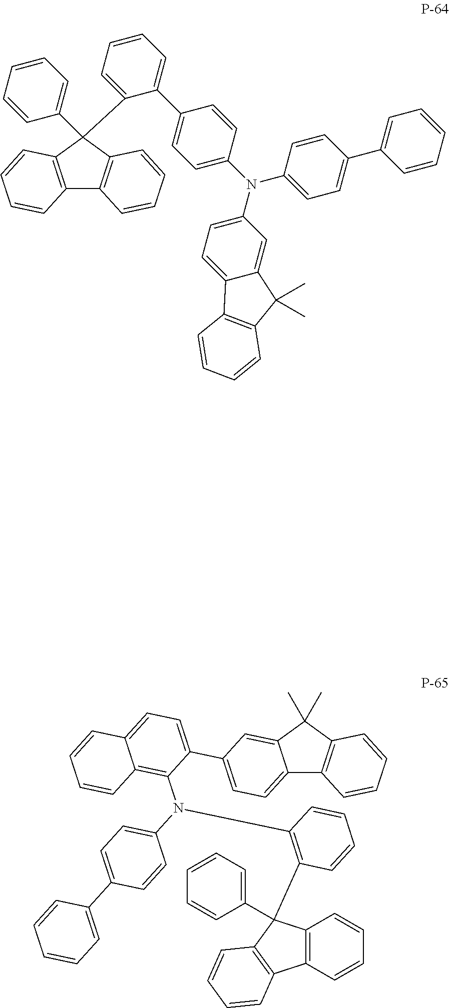 [SMILES: CC1(C)c2ccccc2-c2ccc(N(c3ccc(-c4ccccc4)cc3)c3ccc(-c4ccccc4C4(c5ccccc5)c5ccccc5-c5ccccc54)cc3)cc21 ; CC1(C)c2ccccc2-c2ccc(-c3ccc4ccccc4c3N(c3ccc(-c4ccccc4)cc3)c3ccccc3C3(c4ccccc4)c4ccccc4-c4ccccc43)cc21]

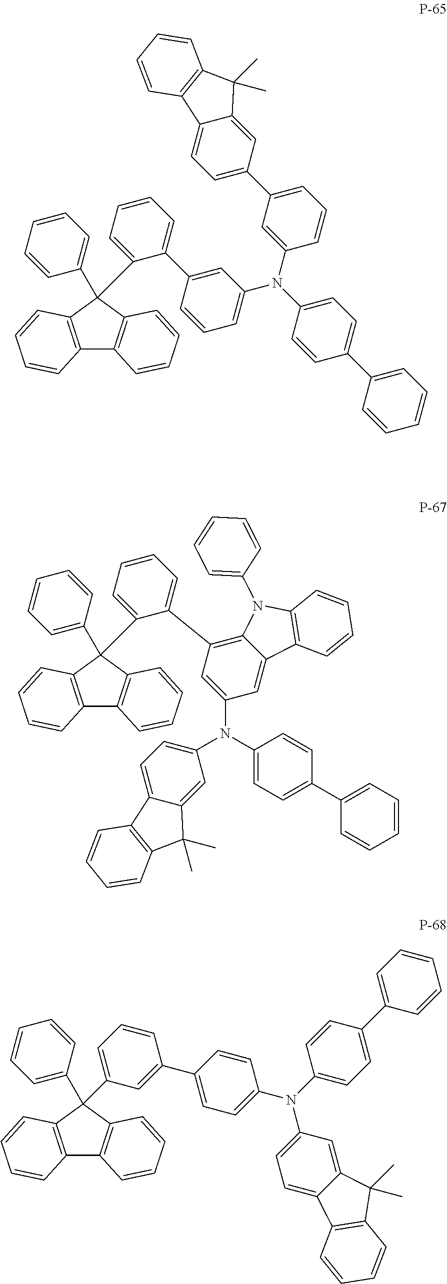 [SMILES: CC1(C)c2ccccc2-c2ccc(-c3cccc(N(c4ccc(-c5ccccc5)cc4)c4cccc(-c5ccccc5C5(c6ccccc6)c6ccccc6-c6ccccc65)c4)c3)cc21 ; CC1(C)c2ccccc2-c2ccc(N(c3ccc(-c4ccccc4)cc3)c3cc(-c4ccccc4C4(c5ccccc5)c5ccccc5-c5ccccc54)c4c(c3)c3ccccc3n4-c3ccccc3)cc21 ; CC1(C)c2ccccc2-c2ccc(N(c3ccc(-c4ccccc4)cc3)c3ccc(-c4cccc(C5(c6ccccc6)c6ccccc6-c6ccccc65)c4)cc3)cc21]

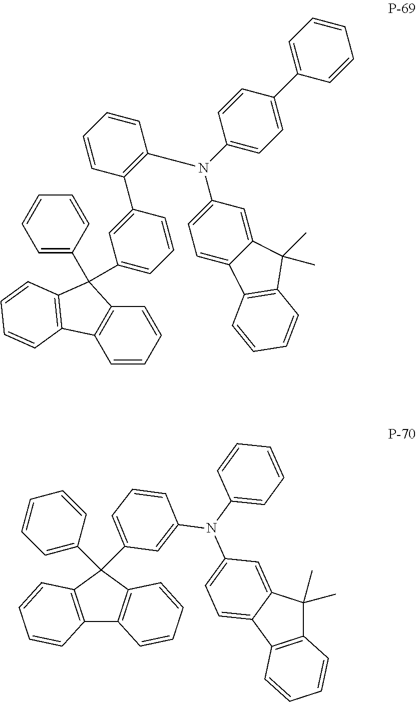 [SMILES: CC1(C)c2ccccc2-c2ccc(N(c3ccc(-c4ccccc4)cc3)c3ccccc3-c3cccc(C4(c5ccccc5)c5ccccc5-c5ccccc54)c3)cc21 ; CC1(C)c2ccccc2-c2ccc(N(c3ccccc3)c3cccc(C4(c5ccccc5)c5ccccc5-c5ccccc54)c3)cc21]

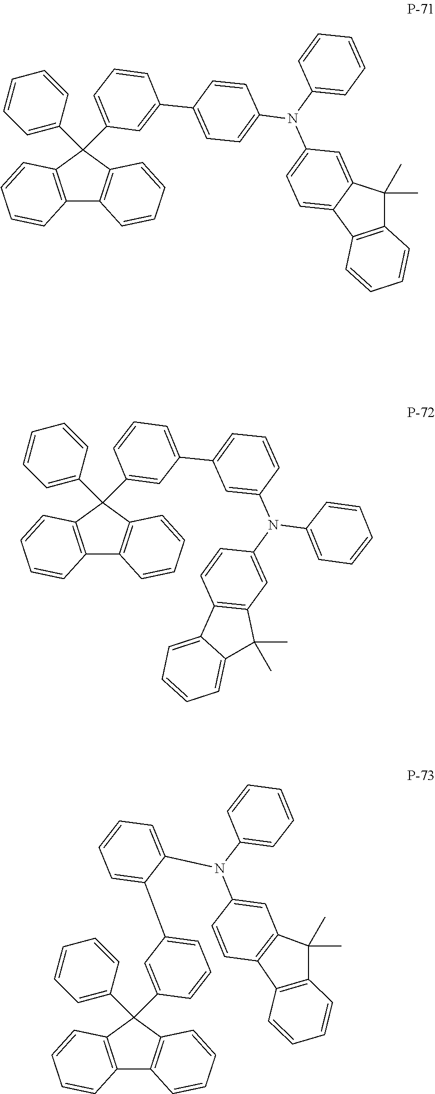 [SMILES: CC1(C)c2ccccc2-c2ccc(N(c3ccccc3)c3ccc(-c4cccc(C5(c6ccccc6)c6ccccc6-c6ccccc65)c4)cc3)cc21 ; CC1(C)c2ccccc2-c2ccc(N(c3ccccc3)c3cccc(-c4cccc(C5(c6ccccc6)c6ccccc6-c6ccccc65)c4)c3)cc21 ; CC1(C)c2ccccc2-c2ccc(N(c3ccccc3)c3ccccc3-c3cccc(C4(c5ccccc5)c5ccccc5-c5ccccc54)c3)cc21]

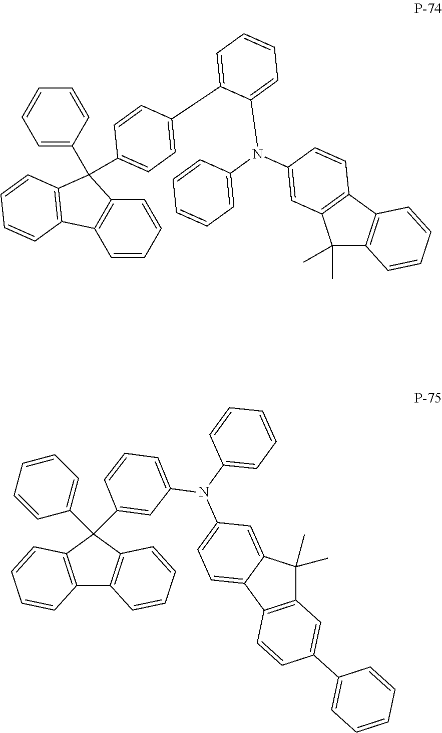 [SMILES: CC1(C)c2ccccc2-c2ccc(N(c3ccccc3)c3ccccc3-c3ccc(C4(c5ccccc5)c5ccccc5-c5ccccc54)cc3)cc21 ; CC1(C)c2cc(-c3ccccc3)ccc2-c2ccc(N(c3ccccc3)c3cccc(C4(c5ccccc5)c5ccccc5-c5ccccc54)c3)cc21]

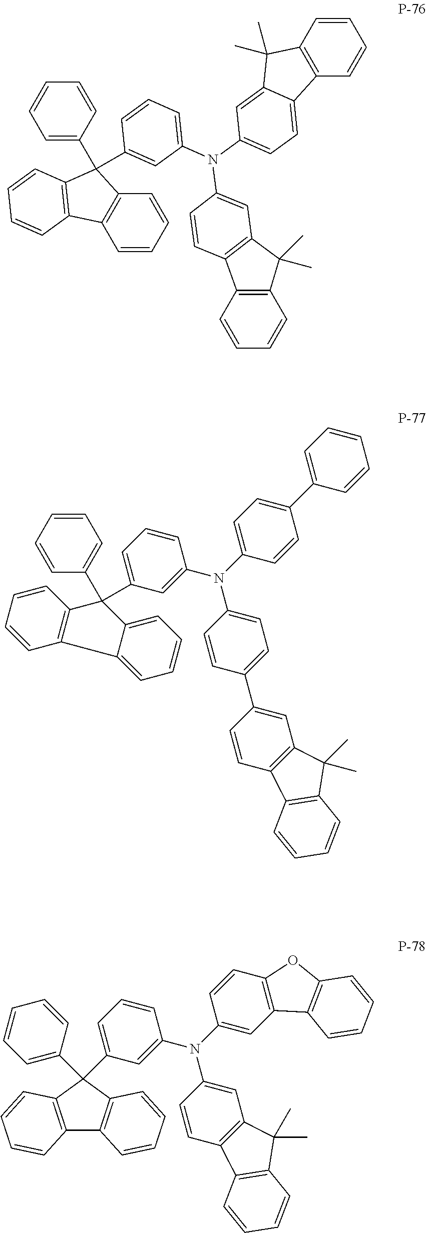 [SMILES: CC1(C)c2ccccc2-c2ccc(N(c3cccc(C4(c5ccccc5)c5ccccc5-c5ccccc54)c3)c3ccc4c(c3)C(C)(C)c3ccccc3-4)cc21 ; CC1(C)c2ccccc2-c2ccc(-c3ccc(N(c4ccc(-c5ccccc5)cc4)c4cccc(C5(c6ccccc6)c6ccccc6-c6ccccc65)c4)cc3)cc21 ; CC1(C)c2ccccc2-c2ccc(N(c3cccc(C4(c5ccccc5)c5ccccc5-c5ccccc54)c3)c3ccc4oc5ccccc5c4c3)cc21]

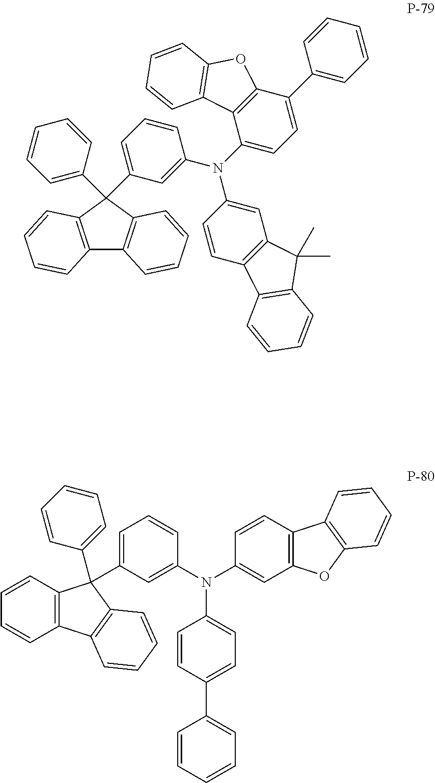 [SMILES: CC1(C)c2ccccc2-c2ccc(N(c3cccc(C4(c5ccccc5)c5ccccc5-c5ccccc54)c3)c3ccc(-c4ccccc4)c4oc5ccccc5c34)cc21 ; c1ccc(-c2ccc(N(c3cccc(C4(c5ccccc5)c5ccccc5-c5ccccc54)c3)c3ccc4c(c3)oc3ccccc34)cc2)cc1]

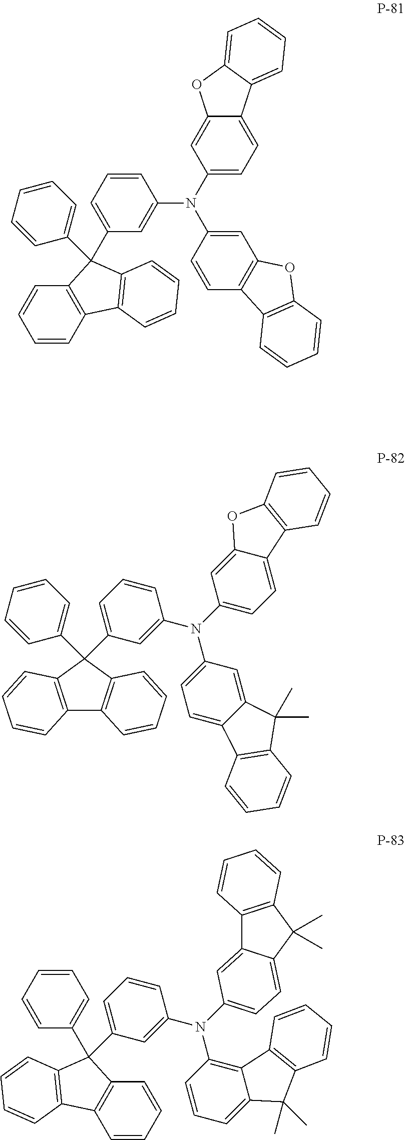

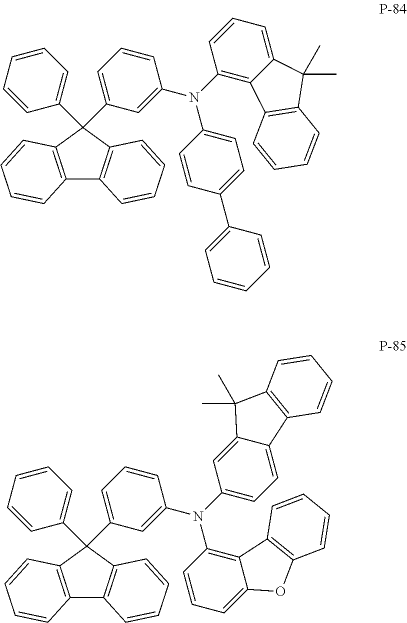 [SMILES: CC1(C)c2ccccc2-c2c(N(c3ccc(-c4ccccc4)cc3)c3cccc(C4(c5ccccc5)c5ccccc5-c5ccccc54)c3)cccc21 ; CC1(C)c2ccccc2-c2ccc(N(c3cccc(C4(c5ccccc5)c5ccccc5-c5ccccc54)c3)c3cccc4oc5ccccc5c34)cc21]

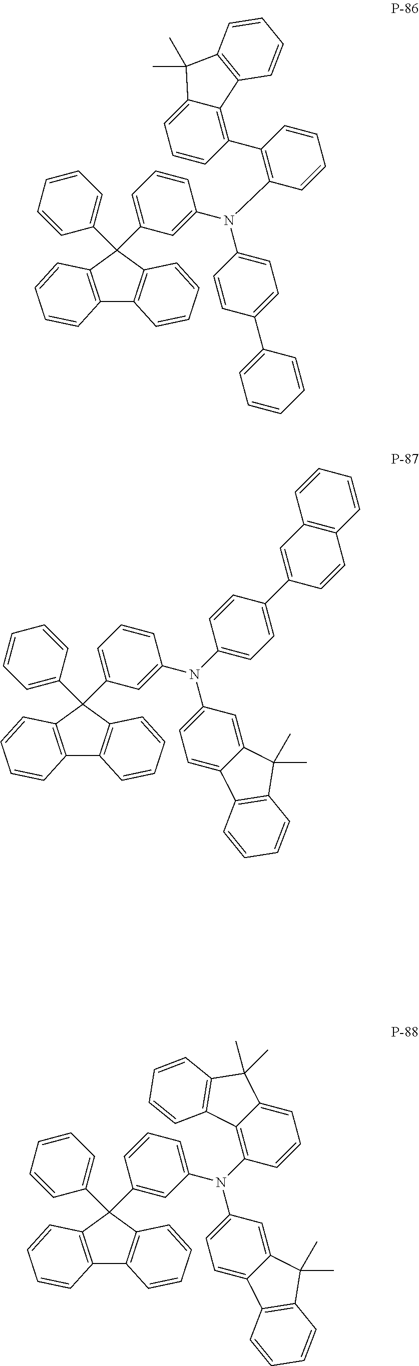 [SMILES: CC1(C)c2ccccc2-c2c(-c3ccccc3N(c3ccc(-c4ccccc4)cc3)c3cccc(C4(c5ccccc5)c5ccccc5-c5ccccc54)c3)cccc21 ; CC1(C)c2ccccc2-c2ccc(N(c3ccc(-c4ccc5ccccc5c4)cc3)c3cccc(C4(c5ccccc5)c5ccccc5-c5ccccc54)c3)cc21 ; CC1(C)c2ccccc2-c2ccc(N(c3cccc(C4(c5ccccc5)c5ccccc5-c5ccccc54)c3)c3cccc4c3-c3ccccc3C4(C)C)cc21]

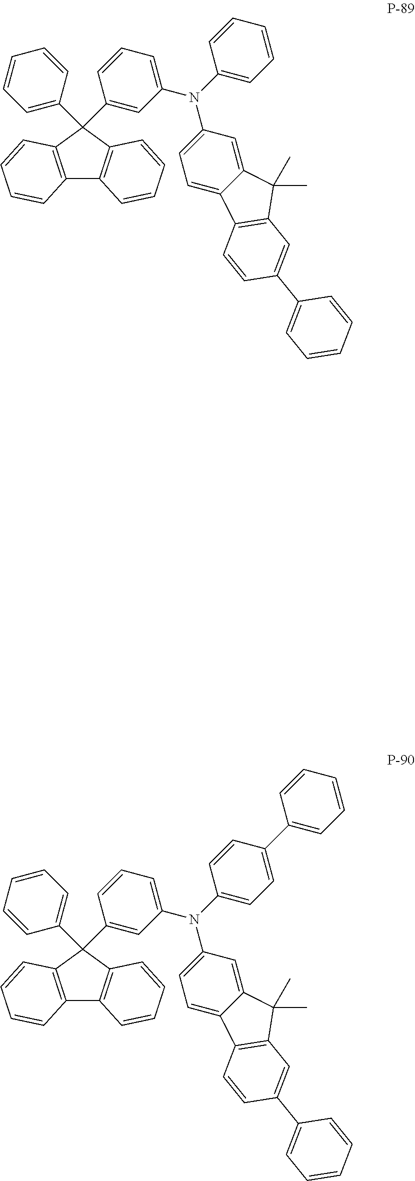 [SMILES: CC1(C)c2cc(-c3ccccc3)ccc2-c2ccc(N(c3ccccc3)c3cccc(C4(c5ccccc5)c5ccccc5-c5ccccc54)c3)cc21 ; CC1(C)c2cc(-c3ccccc3)ccc2-c2ccc(N(c3ccc(-c4ccccc4)cc3)c3cccc(C4(c5ccccc5)c5ccccc5-c5ccccc54)c3)cc21]

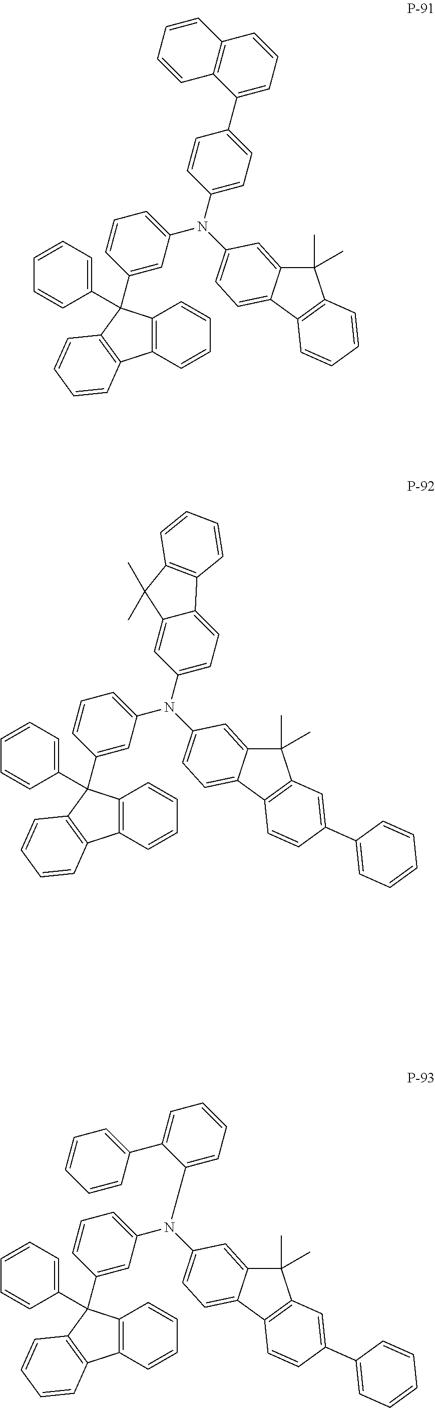 [SMILES: CC1(C)c2ccccc2-c2ccc(N(c3ccc(-c4cccc5ccccc45)cc3)c3cccc(C4(c5ccccc5)c5ccccc5-c5ccccc54)c3)cc21 ; CC1(C)c2ccccc2-c2ccc(N(c3cccc(C4(c5ccccc5)c5ccccc5-c5ccccc54)c3)c3ccc4c(c3)C(C)(C)c3cc(-c5ccccc5)ccc3-4)cc21 ; CC1(C)c2cc(-c3ccccc3)ccc2-c2ccc(N(c3cccc(C4(c5ccccc5)c5ccccc5-c5ccccc54)c3)c3ccccc3-c3ccccc3)cc21]

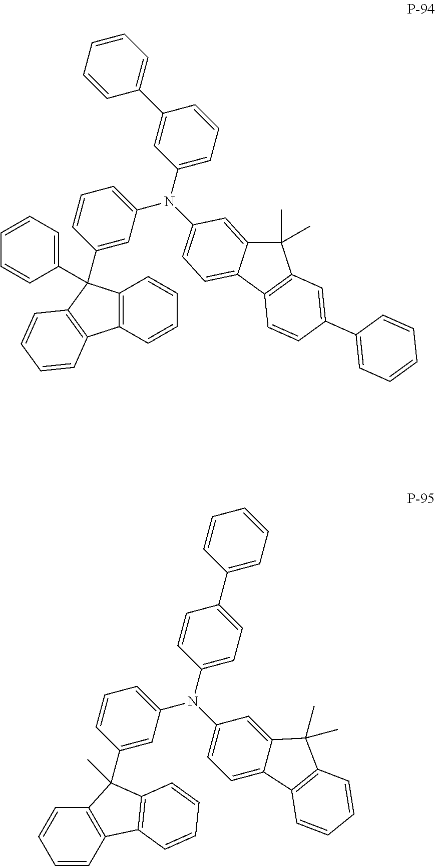 [SMILES: CC1(C)c2cc(-c3ccccc3)ccc2-c2ccc(N(c3cccc(-c4ccccc4)c3)c3cccc(C4(c5ccccc5)c5ccccc5-c5ccccc54)c3)cc21 ; CC1(C)c2ccccc2-c2ccc(N(c3ccc(-c4ccccc4)cc3)c3cccc(C4(C)c5ccccc5-c5ccccc54)c3)cc21]

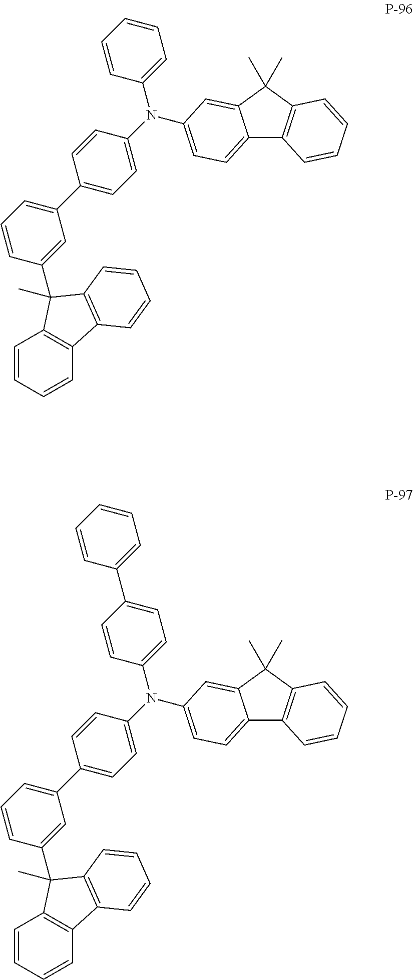 [SMILES: CC1(C)c2ccccc2-c2ccc(N(c3ccccc3)c3ccc(-c4cccc(C5(C)c6ccccc6-c6ccccc65)c4)cc3)cc21 ; CC1(C)c2ccccc2-c2ccc(N(c3ccc(-c4ccccc4)cc3)c3ccc(-c4cccc(C5(C)c6ccccc6-c6ccccc65)c4)cc3)cc21]

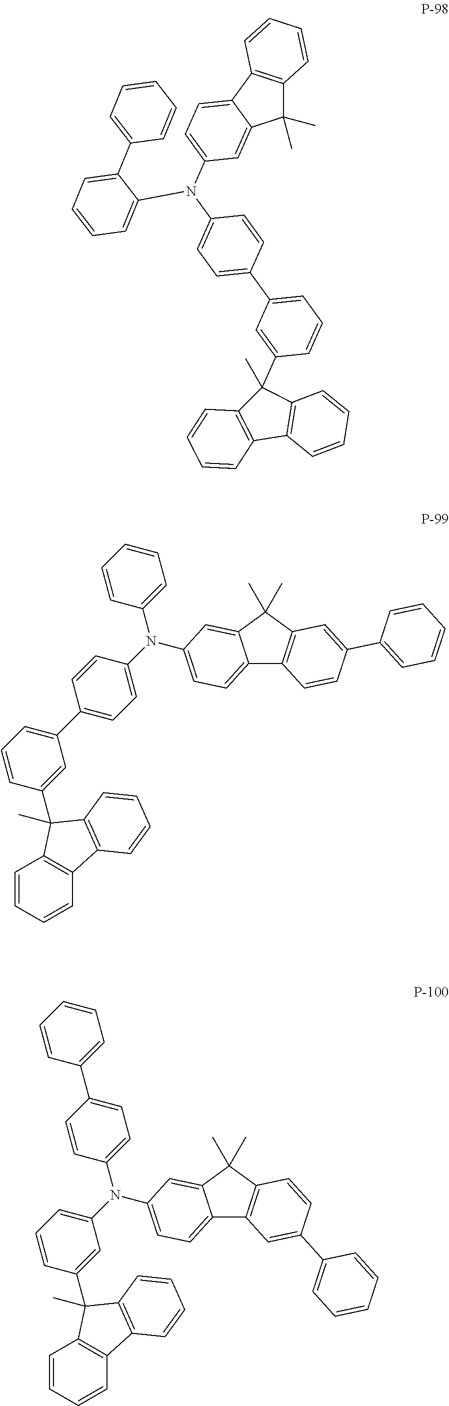 [SMILES: CC1(C)c2ccccc2-c2ccc(N(c3ccc(-c4cccc(C5(C)c6ccccc6-c6ccccc65)c4)cc3)c3ccccc3-c3ccccc3)cc21 ; CC1(C)c2cc(-c3ccccc3)ccc2-c2ccc(N(c3ccccc3)c3ccc(-c4cccc(C5(C)c6ccccc6-c6ccccc65)c4)cc3)cc21 ; CC1(C)c2ccc(-c3ccccc3)cc2-c2ccc(N(c3ccc(-c4ccccc4)cc3)c3cccc(C4(C)c5ccccc5-c5ccccc54)c3)cc21]

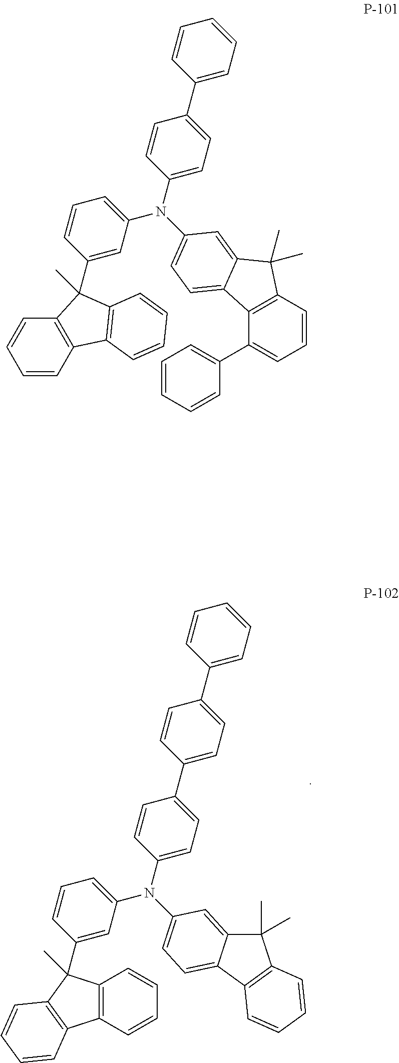 [SMILES: CC1(C)c2cc(N(c3ccc(-c4ccccc4)cc3)c3cccc(C4(C)c5ccccc5-c5ccccc54)c3)ccc2-c2c(-c3ccccc3)cccc21 ; CC1(C)c2ccccc2-c2ccc(N(c3ccc(-c4ccc(-c5ccccc5)cc4)cc3)c3cccc(C4(C)c5ccccc5-c5ccccc54)c3)cc21]

When the first hole transport layer 1411a or the first auxiliary emission layer 1411b includes the above-described first compound and the first hole transport layer 1411a meets the above-described thickness and doping conditions, an organic electronic device having superior efficiency or increased lifespan may be provided.

The second hole transport layer or the second auxiliary emission layer may include the second compound represented by Formula 1. In another example, each of the second hole transport layer and the second auxiliary emission layer may include the second compound represented by Formula 1.

When the second hole transport layer or the second auxiliary emission layer includes the above-described second compound and the second hole transport layer meets the above-described thickness and doping conditions, an organic electronic device having superior efficiency or increased lifespan may be provided.

The third hole transport layer or the third auxiliary emission layer may include the third compound represented by Formula 1. In another example, each of the third hole transport layer and the third auxiliary emission layer may include the third compound represented by Formula 1.

When the third hole transport layer or the third auxiliary emission layer includes the third compound and the third hole transport layer meets the above-described thickness and doping conditions, an organic electronic device having superior efficiency or increased lifespan may be provided.

The fourth hole transport layer 1441a or the fourth auxiliary emission layer 1441b may include the fifth compound represented by Formula 1. In another example, the fourth hole transport layer 1441a and a fourth auxiliary emission layer 1441b may include the fifth compound represented by Formula 1.

When the fourth hole transport layer or the fourth auxiliary emission layer includes the fifth compound and the fourth hole transport layer meets the above-described thickness and doping conditions, an organic electronic device having superior efficiency or increased lifespan may be provided.

Regarding the second compound, the third compound, and the fifth compound, what has been described above for the first compound may equally be applied, unless clearly stated otherwise.

In another example, the first hole transport layer 1411a or the first auxiliary emission layer 1411b may include at least one of the first compound or the fourth compound. In another example, the first hole transport layer 1411a or the first auxiliary emission layer 1411b may include at least one of the first compound or the fourth compound.

The first hole transport layer 1411a or the first auxiliary emission layer 1411b includes at least one of the first compound or the fourth compound and the first hole transport layer 1411a meets the above-described thickness and doping conditions, an organic electronic device having superior efficiency or increased lifespan may be provided.

In another example, the fourth hole transport layer 1441a or the fourth auxiliary emission layer 1441b may include at least one of the fifth compound or the sixth compound. In another example, the fourth hole transport layer 1441a and the fourth auxiliary emission layer 1441b may include at least one of the fifth compound or the sixth compound.

When the fourth hole transport layer 1441a or the fourth auxiliary emission layer 1441b includes at least one of the fifth compound or the sixth compound and the fourth hole transport layer 1441a meets the above-described thickness and doping conditions, an organic electronic device having superior efficiency or increased lifespan may be provided.

The fourth compound may include a radical of a compound represented by the following Formula A or Formula B, and be represented by at least one of compounds represented by the following Formula C or Formula D.

[Formulas A, B, C, and D]

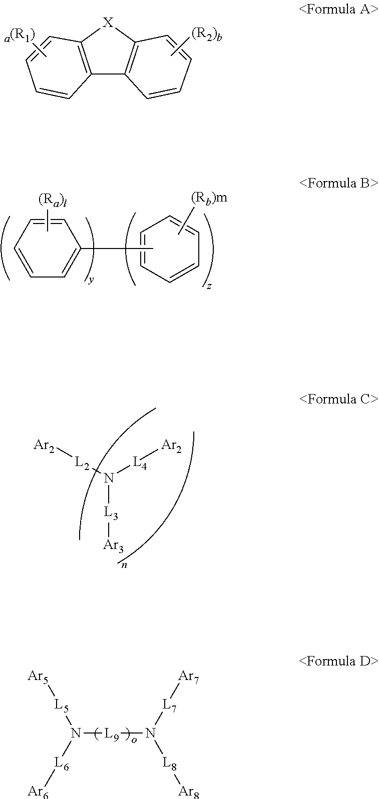

In embodiments of the present disclosure, the description that “any compound includes a radical of a compound represented by Formula A or Formula B and is represented by Formula C or Formula D” may mean that the compound has a structure represented by Formula C or Formula D and is an n-valent radical of a compound in which one or more of substituents and linkages in Formula C or Formula D are represented by Formula A or Formula B (here, n is an integer equal to or greater than 1). On the other hand, the description that “any compound includes a radical of a compound represented by Formula A or Formula B and is represented by Formula C or Formula D” may not mean that the compound includes the radical of the compound represented by Formula A or Formula B in the form of an n-valent radical but may mean that the compound does not include the radical of the compound, represented by Formula A or Formula B, in the form of an n-valent radical but includes the radical of the compound represented by Formula A or Formula B in a state in which the radical is covalently bonded an element of a compound represented by Formula C or Formula D.

Hereinafter, Formula A will be described.

Each of a and b is independently an integer from 0 to 4.

X is O, S, CR′R″, or N-L₁-Ar₁.

R₁ and R₂ are respectively and independently selected from the group consisting of deuterium; tritium; a halogen; a cyano group; a nitro group; a C₆-C₆₀ aryl group; a fluorenyl group; a C₂-C₆₀ heterocyclic group including at least one hetero atom of O, N, S, Si, or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; or a C₆-C₃₀ aryloxy group. R¹s and R²s may be bonded to form rings, respectively.

When R₁ or R₂ is an aryl group, R₁ or R₂ may be, for example, a C₆-C₆₀ aryl group, a C₆-C₄₀ aryl group, a C₆-C₂₅ aryl group, or a C₆-C₁₀ aryl group.

When R₁ or R₂ is a heterocyclic group, R₁ or R₂ may be, for example, a C₂-C₆₀ heterocyclic group, a C₂-C₄₀ heterocyclic group, or a C₂-C₂₀ heterocyclic group.

Each of R′ and R″ is independently selected from the group consisting of hydrogen; deuterium; a C₆-C₆₀ aryl group; a fluorenyl group; a C₂-C₆₀ heterocyclic group including at least one hetero atom of O, N, S, Si, or P; or a fused ring group of a C₃-C₆₀ aliphatic ring and a C₆-C₆₀ aromatic ring. R's and R″s may be bonded to form rings, respectively.

When R′ or R″ is an aryl group, R′ or R″ may be, for example, a C₆-C₆₀ aryl group, a C₆-C₄₀ aryl group, a C₆-C₂₅ aryl group, or a C₆-C₁₀ aryl group.

When R′ or R″ is a heterocyclic group, R′ or R″ may be, for example, a C₂-C₆₀ heterocyclic group, a C₂-C₄₀ heterocyclic group, or a C₂-C₂₀ heterocyclic group.

L₁ is selected from the group consisting of a single bond; a C₆-C₆₀ arylene group; a fluorenylene group; a C₂-C₆₀ heterocyclic group including at least one hetero atom of O, N, S, Si, or P; or a fused ring group of a C₃-C₆₀ aliphatic ring and a C₆-C₆₀ aromatic ring.

When L₁ is an arylene group, L₁ may be, for example, a C₆-C₆₀ arylene group, a C₆-C₄₀ arylene group, a C₆-C₂₅ arylene group, or a C₆-C₁₀ arylene group.

When L₁ is a heterocyclic group, L₁ may be, for example, a C₂-C₆₀ heterocyclic group, a C₂-C₄₀ heterocyclic group, or a C₂-C₂₀ heterocyclic group.

Ar₁ is selected from the group consisting of a C₆-C₆₀ aryl group; a fluorenyl group; a C₂-C₆₀ heterocyclic group including at least one hetero atom of O, N, S, Si, or P; or a fused ring group of a C₃-C₆₀ aliphatic ring and a C₆-C₆₀ aromatic ring.

When Ar₁ is an aryl group, Ar₁ may be, for example, a C₆-C₆₀ aryl group, a C₆-C₄₀ aryl group, a C₆-C₂₅ aryl group, or a C₆-C₁₀ aryl group.

When Ar₁ is a heterocyclic group, Ar₁ may be, for example, a C₂-C₆₀ heterocyclic group, a C₂-C₄₀ heterocyclic group, or a C₂-C₂₀ heterocyclic group.

Hereinafter, Formula B will be described.

l is an integer from 0 to 5.

m is an integer from 0 to 4.

each y and z is an integer from 0 to 4, where y+z is not zero (0).

Each of Rₐ and R_{b} is independently selected from the group consisting of deuterium; tritium; a halogen; a cyano group; a nitro group; a C₆-C₆₀ aryl group; a fluorenyl group; a C₂-C₆₀ heterocyclic group including at least one hetero atom of O, N, S, Si, or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; or a C₆-C₃₀ aryloxy group. Rₐs and R_{b}s may be bonded to form rings, respectively.

When Rₐ or R_{b} is an aryl group, Rₐ or R_{b} may be, for example, a C₆-C₆₀ aryl group, a C₆-C₄₀ aryl group, a C₆-C₂₅ aryl group, or a C₆-C₁₀ aryl group.

When Rₐ or R_{b} is a heterocyclic group, Rₐ or R_{b} may be, for example, a C₂-C₆₀ heterocyclic group, a C₂-C₄₀ heterocyclic group, or a C₂-C₂₀ heterocyclic group.

Hereinafter, Formula C will be described.

n is 1 or 2.

Ar₂ is a radical of a compound represented by Formula A or a radical of a compound represented by Formula B.

Each of Ar₃ and Ar₄ is independently selected from the group consisting of a C₅-C₆₀ aryl group; a fluorenyl group; a C₂-C₆₀ heterocyclic group including at least one hetero atom of O, N, S, Si, or P; or a fused ring group of a C₃-C₅₀ aliphatic ring and a C₆-C₆₀ aromatic ring.

When Ar₃ or Ar₄ is an aryl group, Ar₃ or Ar₄ may be, for example, a C₆-C₆₀ aryl group, a C₆-C₄₀ aryl group, a C₆-C₂₅ aryl group, or a C₅-C₁₀ aryl group.

Ar₃ or Ar₄ is a heterocyclic group, Ar₃ or Ar₄ may be, for example, a C₂-C₆₀ heterocyclic group, a C₂-C₄₀ heterocyclic group, or a C₂-C₂₀ heterocyclic group.

Each of L₂ to L₄ is independently selected from the group consisting of a single bond; a C₅-C₆₀ arylene group; a fluorenylene group; or a C₂-C₆₀ heterocyclic group including at least one hetero atom of O, N, S, Si, or P.

When one of L₂ to L₄ is an arylene group, one of L₂ to L₄ may be, for example, a C₆-C₆₀ arylene group, a C₆-C₄₀ arylene group, a C₅-C₂₅ arylene group, or a C₆-C₁₀ arylene group.

When one of L₂ to L₄ is a heterocyclic group, one of L₂ to L₄ may be, for example, a C₂-C₆₀ heterocyclic group, a C₂-C₄₀ heterocyclic group, or a C₂-C₂₀ heterocyclic group.

Hereinafter, Formula D will be described.

o is an integer from 1 to 4.

Each of Ar₅ to Are is independently selected from the group consisting of a C₅-C₆₀ aryl group; a fluorenyl group; a C₂-C₆₀ heterocyclic group including at least one hetero atom of O, N, S, Si, or P; or a fused ring group of a C₃-C₆₀ aliphatic ring and a C₆-C₆₀ aromatic ring.

When one of Ar₅ to Are is an aryl group, one of Ar₅ to Are may be, for example, a C₆-C₅₀ aryl group, a C₆-C₄₀ aryl group, a C₆-C₂₅ aryl group, or a C₆-C₁₀ aryl group.

When one of Ar₅ to Ar₈ is a heterocyclic group, one of Ar₅ to Are may be, for example, a C₂-C₆₀ heterocyclic group, a C₂-C₄₀ heterocyclic group, or a C₂-C₂₀ heterocyclic group.

Each of L₅ to L₉ is independently selected from the group consisting of a single bond; a C₅-C₆₀ arylene group; a fluorenylene group; a C₂-C₆₀ heterocyclic group including at least one hetero atom of O, N, S, Si, or P; or a fused ring group of a C₃-C₆₀ aliphatic ring and a C₅-C₆₀ aromatic ring. L₉ may be a radical of a compound represented by Formula A or a radical of a compound represented by Formula B.

When one of L₅ to L₉ is an arylene group, one of L₅ to L₉ may be, for example, a C₅-C₆₀ arylene group, a C₆-C₄₀ arylene group, a C₆-C₂₅ arylene group, or a C₆-C₁₀ arylene group.

When one of L₅ to L₉ is a heterocyclic group, one of L₅ to L₉ may be, for example, a C₂-C₅₀ heterocyclic group, a C₂-C₄₀ heterocyclic group, or a C₂-C₂₀ heterocyclic group.

In Formula A to Formula D, each of the aryl group, fluorenyl group, the heterocyclic group, the fused ring group, the alkyl group, the alkenyl group, the alkynyl group, the alkoxyl group, the aryloxy group, the arylene group, and the fluorenylene group may be further substituted with one or more substituents selected from the group consisting of deuterium; a nitro group; a nitrile group; a halogen group; amino group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₅ aryl group; a C₆-C₂₅ aryl group substituted with deuterium; a fluorenyl group; a C₂-C₂₀ heterocyclic group; a C₃-C₂₀ cycloalkyl group; a C₇-C₂₀ aryl alkyl group; or a C₈-C₂₀ aryl alkenyl group. In addition, the further substituted substituents may be bonded to form a ring. Each of the further substituted substituents may be further substituted with one or more substituents selected from the group consisting of deuterium; a nitro group; a nitrile group; a halogen group; an amino group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₅ aryl group; a C₆-C₂₅ aryl group substituted with deuterium; a fluorenyl group; a C₂-C₂₀ heterocyclic group; a C₃-C₂₀ cycloalkyl group; a C₇-C₂₀ aryl alkyl group; or a C₈-C₂₀ aryl alkenyl group. In addition, theses substituents may be bonded to form a ring.

The fourth compound may include a radical of a compound represented by Formula A, be represented by Formula C, and be represented by one of the following Formula H-1 to Formula H-5.

[Formulas H-1 and H-2]

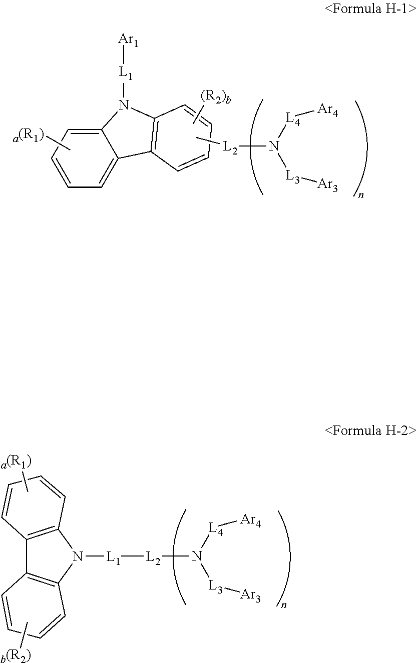

[Formulas H-3, H-4, and H-4]

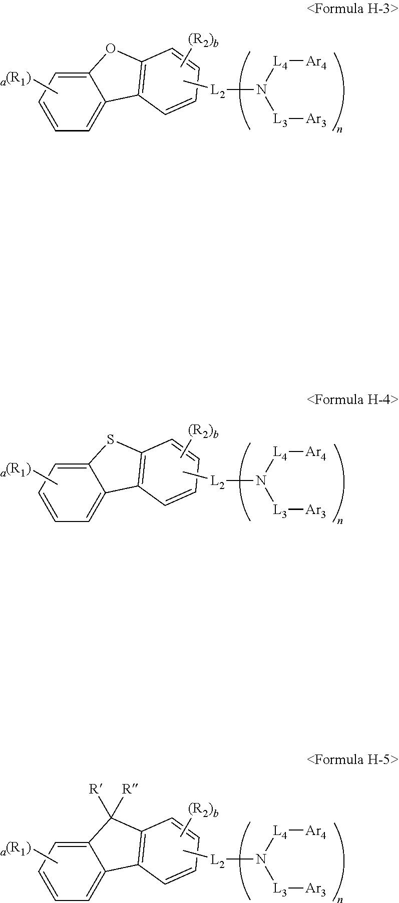

In Formula H-1 to Formula H-5, a, b, n, R₁, R₂, R′, R″, Ar₁, Ar₃, Ar₄, and L₁ to L₄ are the same as a, b, R₁, R₂, R′, R″, Ar₁, Ar₃, Ar₄, and L₁ to L₄ defined in the description of Formula A to Formula D.

The fourth compound may include a radical of a compound represented by Formula A, be represented by Formula D, and be represented by one of the following Formula I-1 to Formula I-3.

[Formulas I-1 and 1-2]

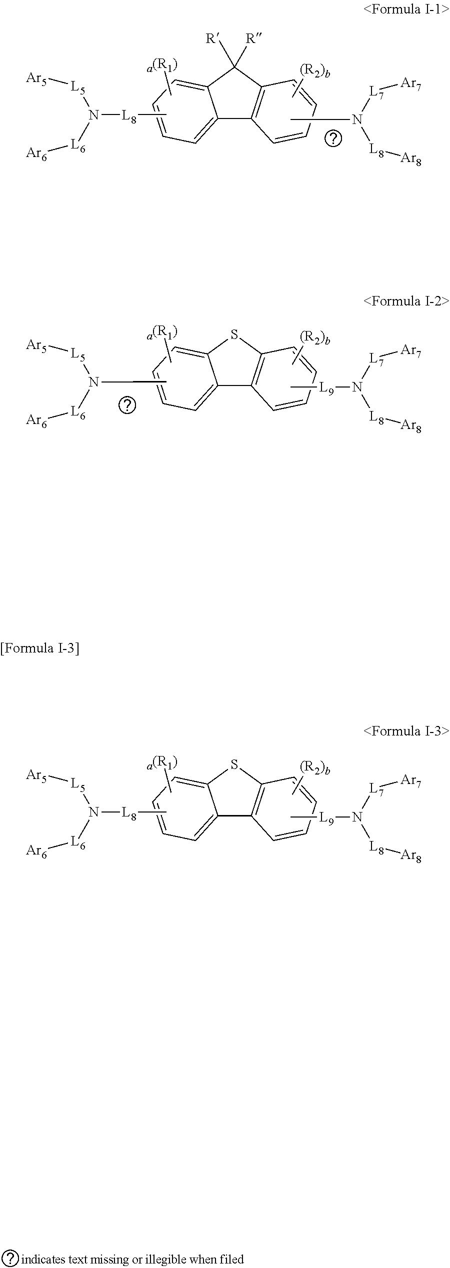

In Formula I-1 to Formula I-3, a, b, R₁, R₂, R′, R″, Ar₅ to Ar₈, and L₅ to L₉ are the same as a, b, R₁, R₂, R′, R″, Ar₅ to Ar₈, and L₅ to L₉ defined in the description of Formula A to Formula D.

The fourth compound may include a radical of a compound represented by Formula A, be represented by Formula D, and be represented by one of the following Formula I-4 to Formula I-6.

[Formulas I-4, I-5, and I-6]

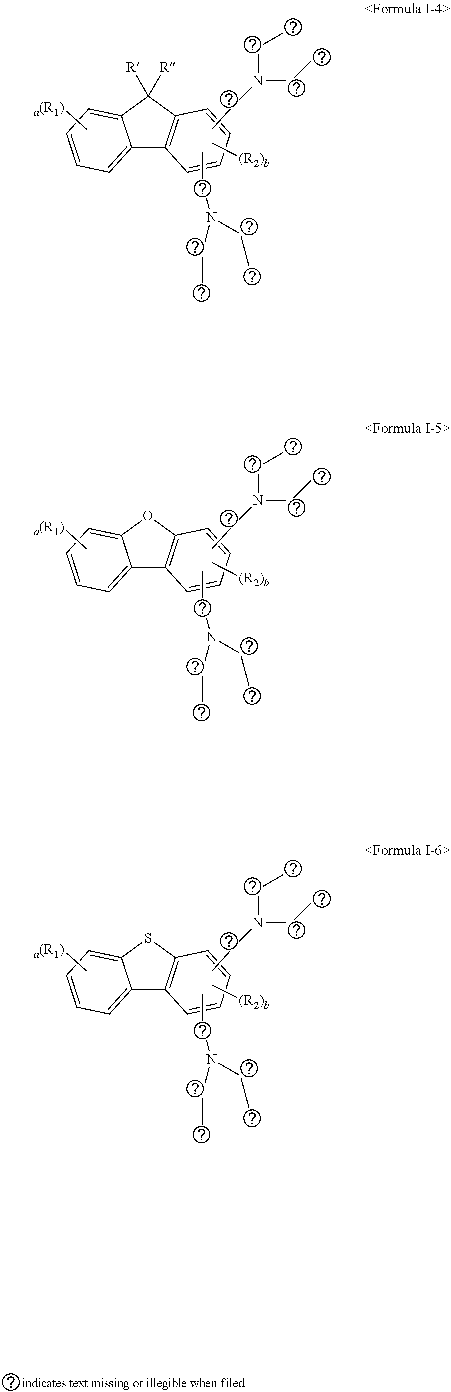

In Formula I-4 to Formula I-6, a, b, R₁, R₂, R′, R″, Ar₅ to Ar₈, and L₅ to L₉ are the same as a, b, R₁, R₂, R′, R″, Ar₅ to Ar₈, and L₅ to L₉ defined in the description of Formula A to Formula D.

The fourth compound may include a radical of a compound represented by Formula B, be represented by Formula C or Formula D, and be represented by one of the following Formula J-1 to Formula J-3.

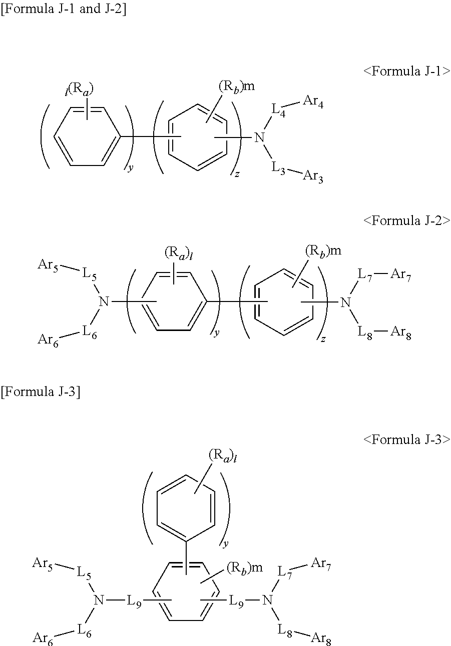

In Formula J-1 to J-3, l, m, n, y, z, Rₐ, R_{b}, Ar₃ to Ar₈, and L₂ to L₉ are the same as l, m, n, Rₐ, R_{b}, Ar₃ to Ar₈, and L₂ to L₉ defined in the description of Formula A to Formula D.

Formula H-1 may be represented by the following Formula H-1-A or Formula H-1-B.

[Formulas H-1-A and H-1-B]

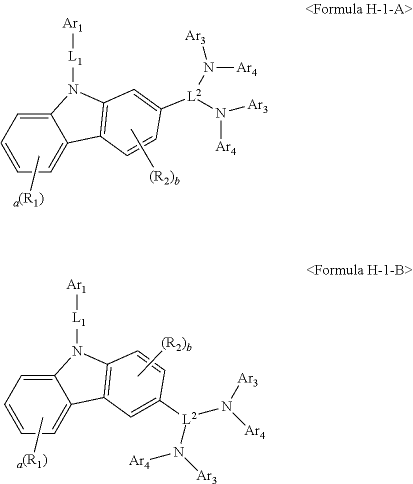

In Formula H-1-A and Formula H-1-B, a, b, R₁, R₂, Ar₁, Ar₃, Ar₄, and L₁ to L₂ are the same as a, b, R₁, R₂, Ar₁, Ar₃, Ar₄, and L₁ to L₂ defined in the description of Formula A to Formula D.

Each of L₁ to L₈ may be independently represented by one of the following Formula b-1 to Formula b-13.

[Formulas b-1, b-2, b-3, b-4, b-5, and b-6]

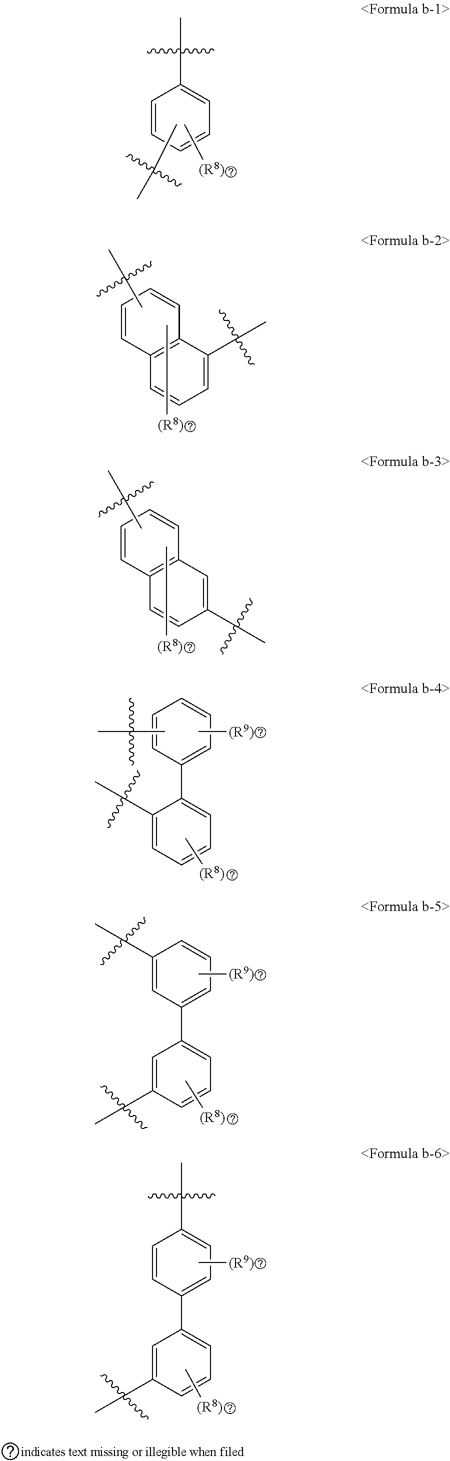

[Formulas b-7, b-8, b-9, and b-10]

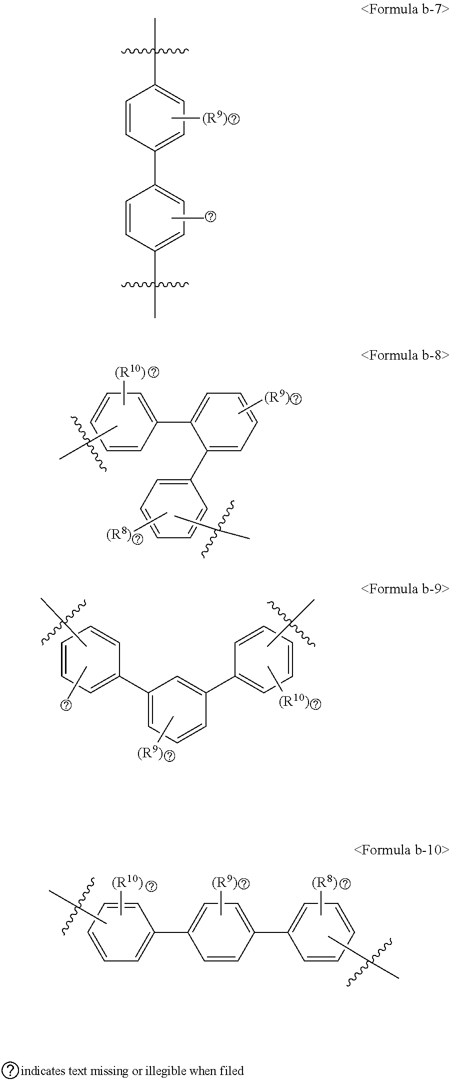

[Formulas b-11, b-12, and b-13]

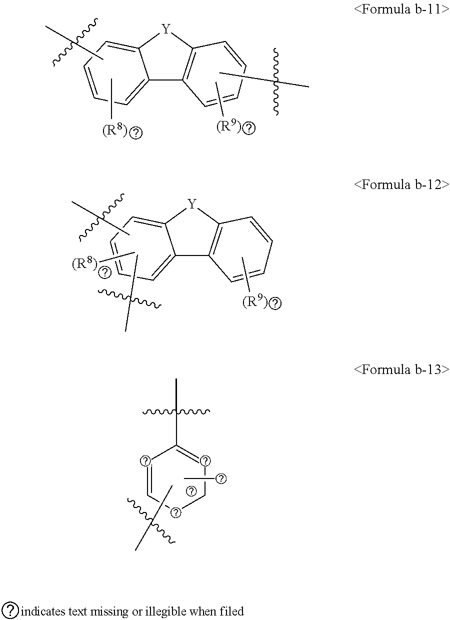

Hereinafter, Formula b-1 to Formula b-13 will be described.

Y is respectively and independently N-L⁶-Ar⁹, O, S, or CR^{d}R^{e}.

L⁶ is the same as L₁ defined in the description of Formula A to Formula D.

Ar⁹ is the same as Ar₁ defined in the description of Formula A to Formula D.

R^{d} and R^{e} are the same as R′ and R″ defined in the description of Formula A to Formula D.

Each a″, c″, d″, and e″ is independently an integer from 0 to 4, and b″ is respectively and independently an integer from 0 to 6.

f″ and g″ are independently an integer from 0 to 3, h″ is an integer from 0 to 2, and i″ is an integer 0 or 1.

Each of R⁸ to R¹⁰ is independently selected from the group consisting of hydrogen; deuterium; tritium; a halogen; a cyano group; a nitro group; a C₆-C₆₀ aryl group; fluorenyl group; a C₂-C₆₀ heterocyclic group including at least one hetero atom selected from the group consisting of O, N, S, Si, or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group, or -L^{a}-N(R^{d}) (R^{e}). R⁸s to R¹⁰s may be bonded to form rings, respectively.

L^{a} is selected from the group consisting of a single bond; a C₆-C₆₀ arylene group; a fluorenylene group; a C₂-C₆₀ heterocyclic group including at least one hetero atom of O, N, S, Si, or P; a fused ring group of a C₃-C₆₀ aliphatic ring and a C₆-C₆₀ aromatic ring; and a C₃-C₆₀ aliphatic hydrocarbon group.

Each of R^{d} and R^{e} is independently selected from the group consisting of a C₆-C₆₀ aryl group; a fluorenyl group; a C₂-C₆₀ heterocyclic group including at least one hetero atom selected from the group consisting of O, N, S, Si, or P; and a fused ring group of a C₃-C₆₀ aliphatic ring and a C₆-C₆₀ aromatic ring.

Each of Z⁴⁹, Z⁵⁰, and Z⁵¹ is independently CR^{f} or N, at least one of Z⁴⁹, Z⁵⁰, or Z⁵¹ is N.

R^{f} is selected from the group consisting of hydrogen; deuterium; tritium; a halogen; a cyano group; a nitro group; a C₆-C₆₀ aryl group; a fluorenyl group; a C₂-C₆₀ heterocyclic group including at least one hetero atom selected from the group consisting of O, N, S, Si, or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; or a C₆-C₃₀ aryloxy group. Adjacent R⁸ and R^{f} may be bonded to form a ring.

The fourth compound may include a radical of a compound represented by Formula A, be represented by Formula C, be one or more of the following compound 1-1 to compound 6-35.

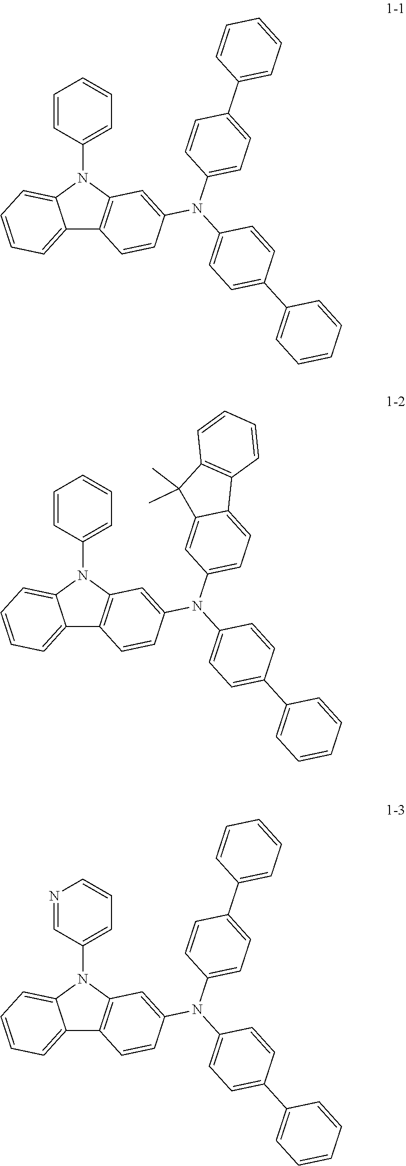

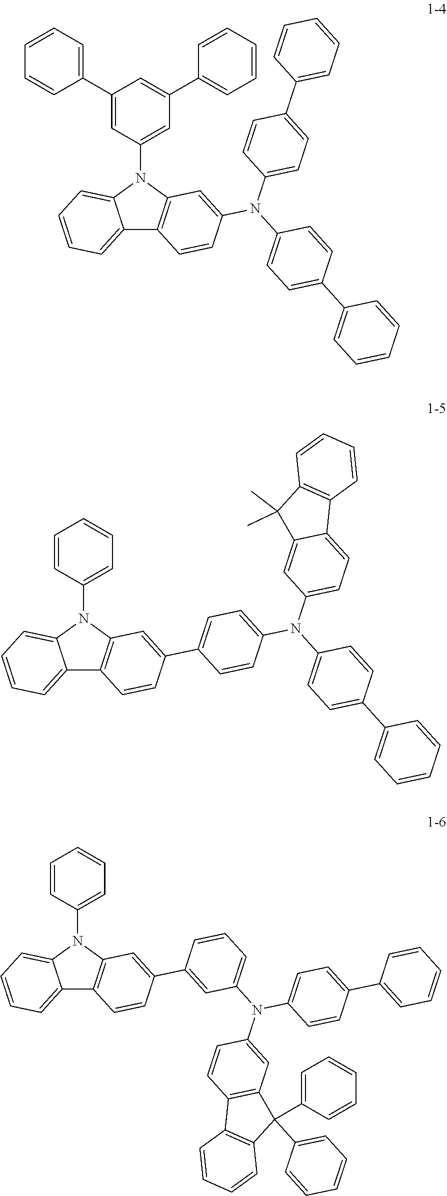

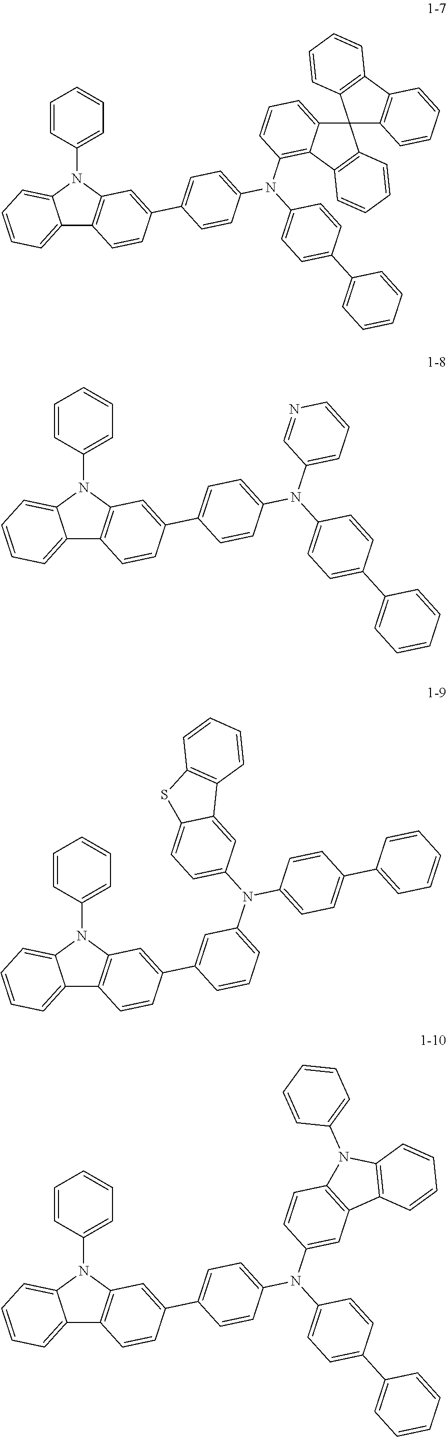

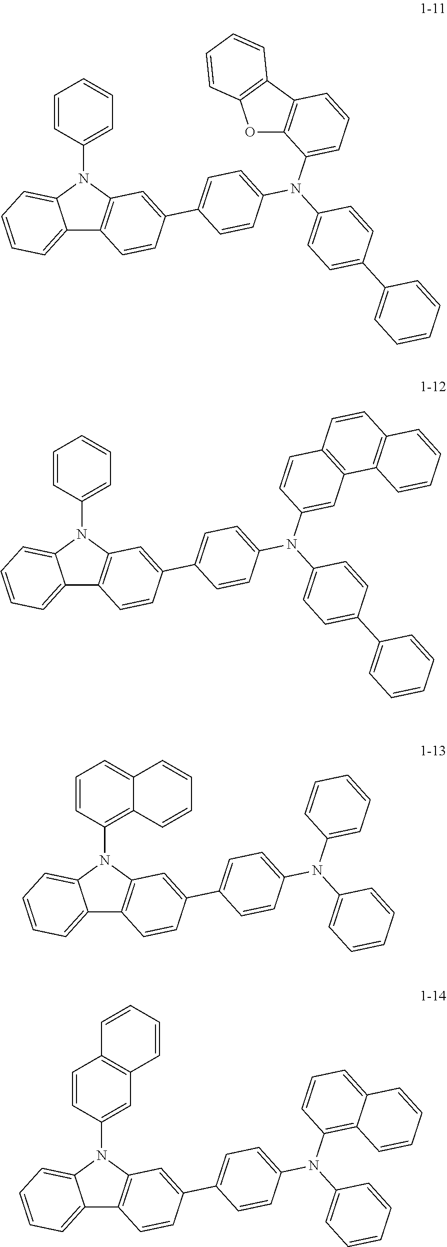

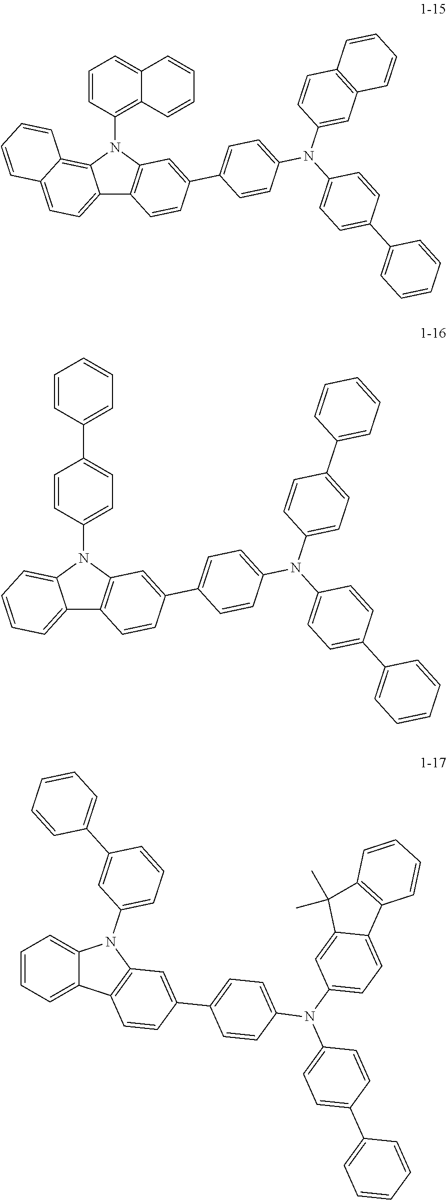

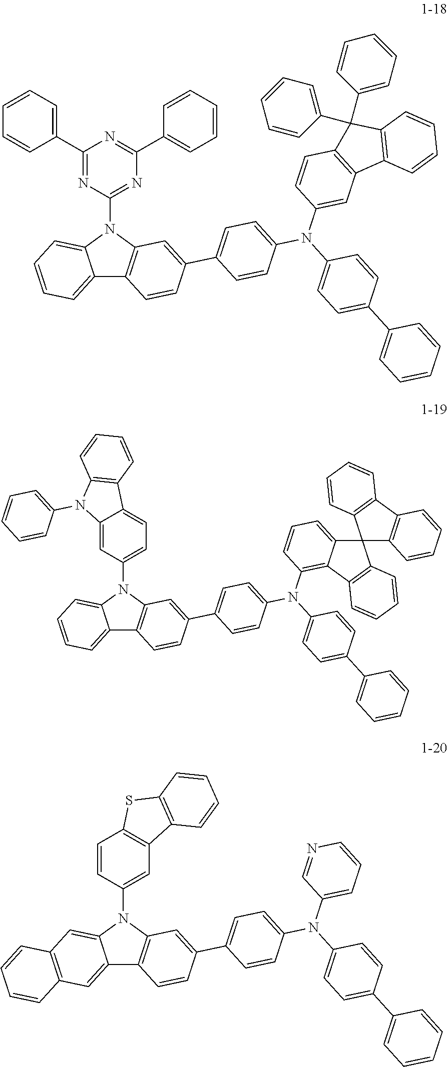

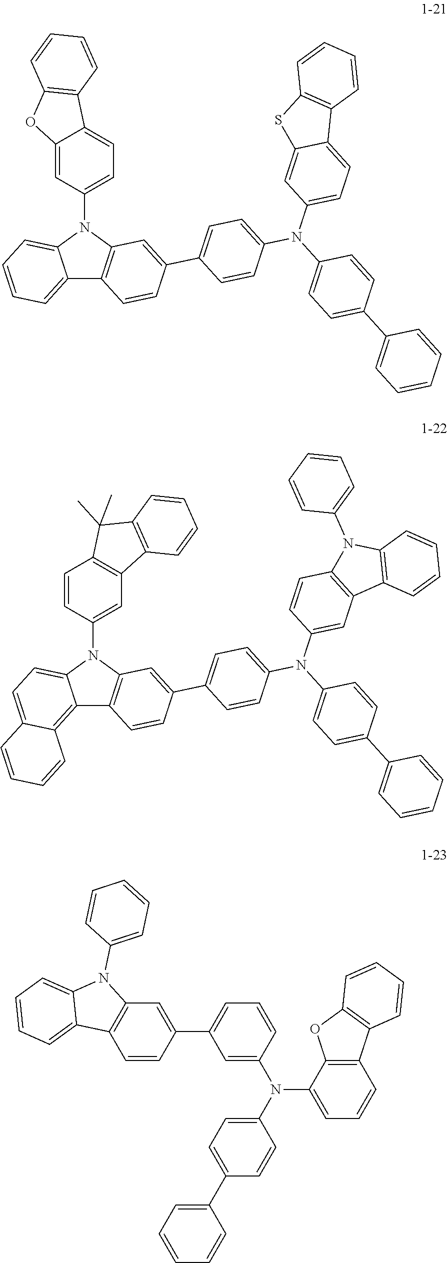

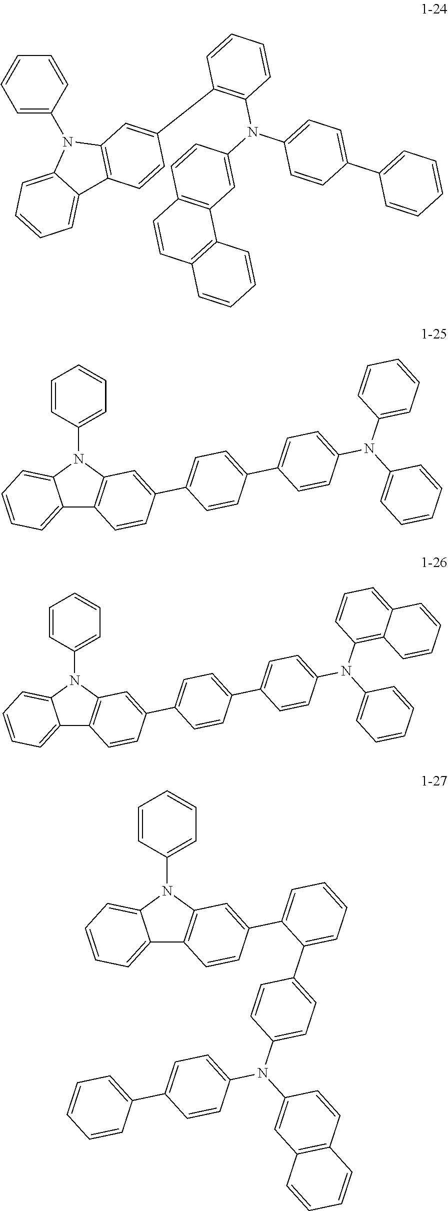

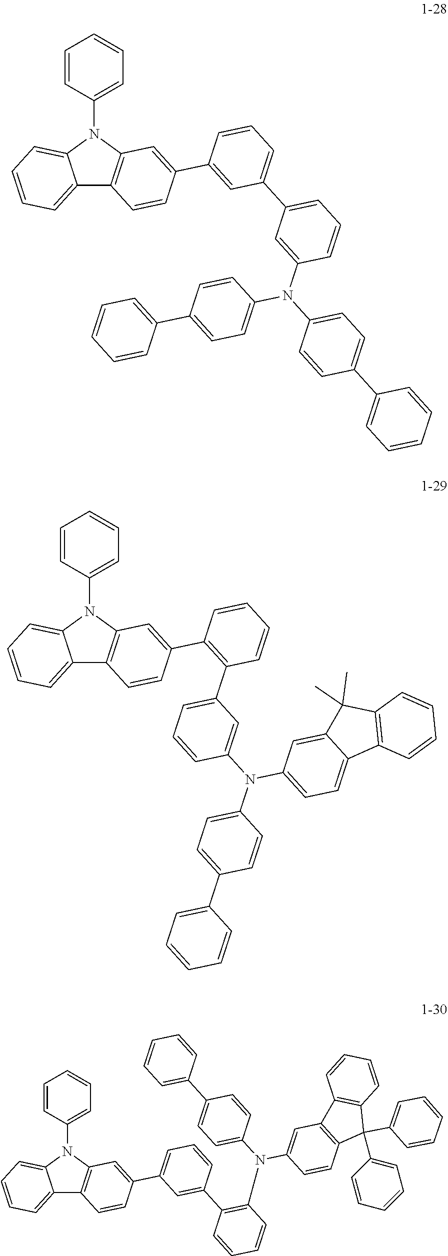

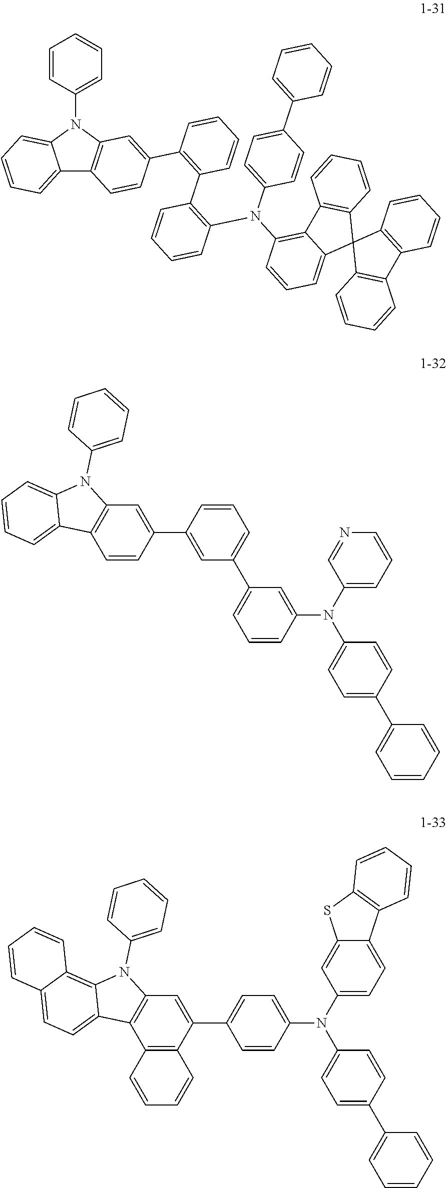

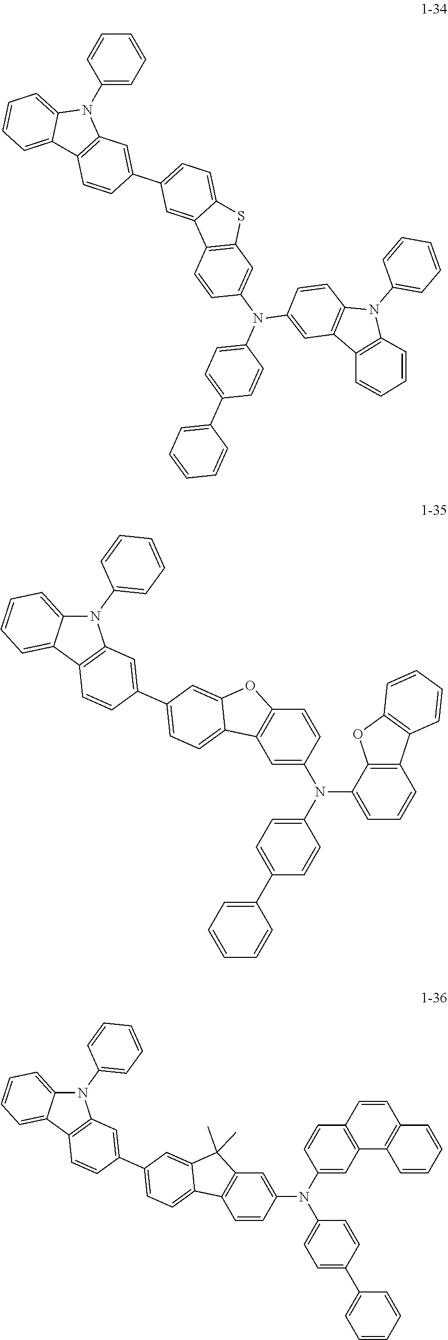

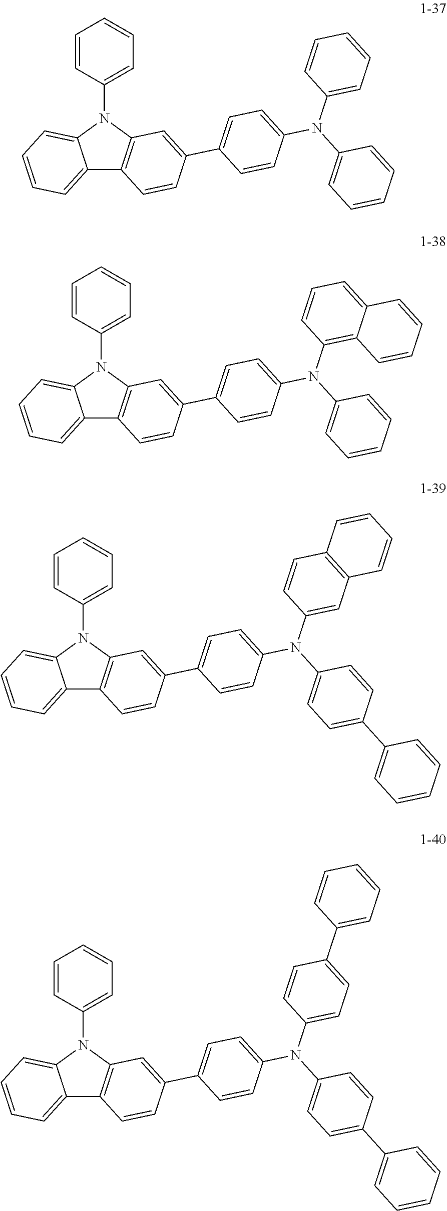

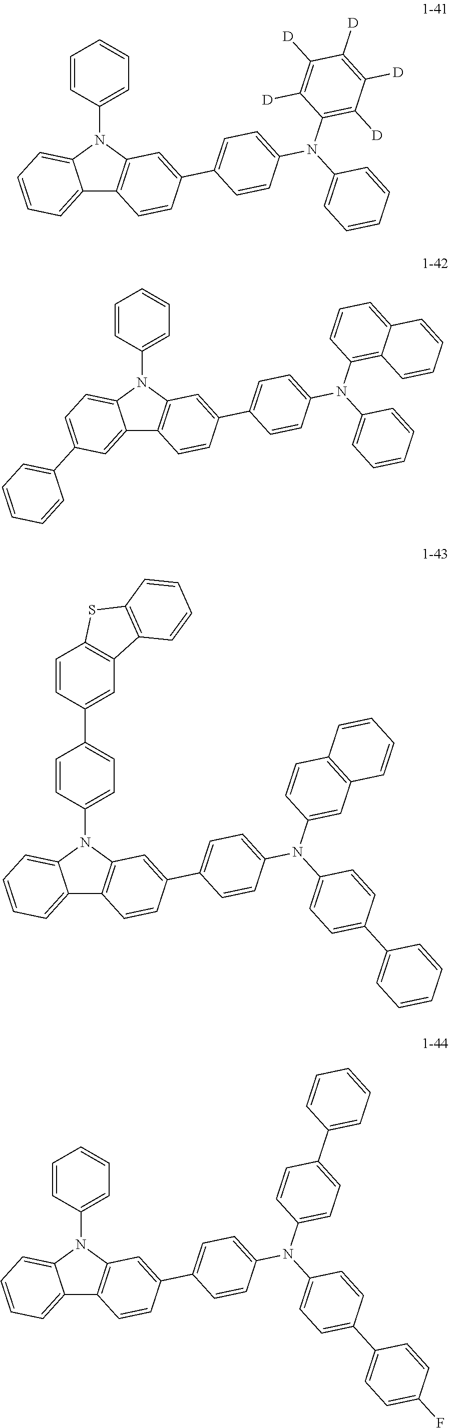

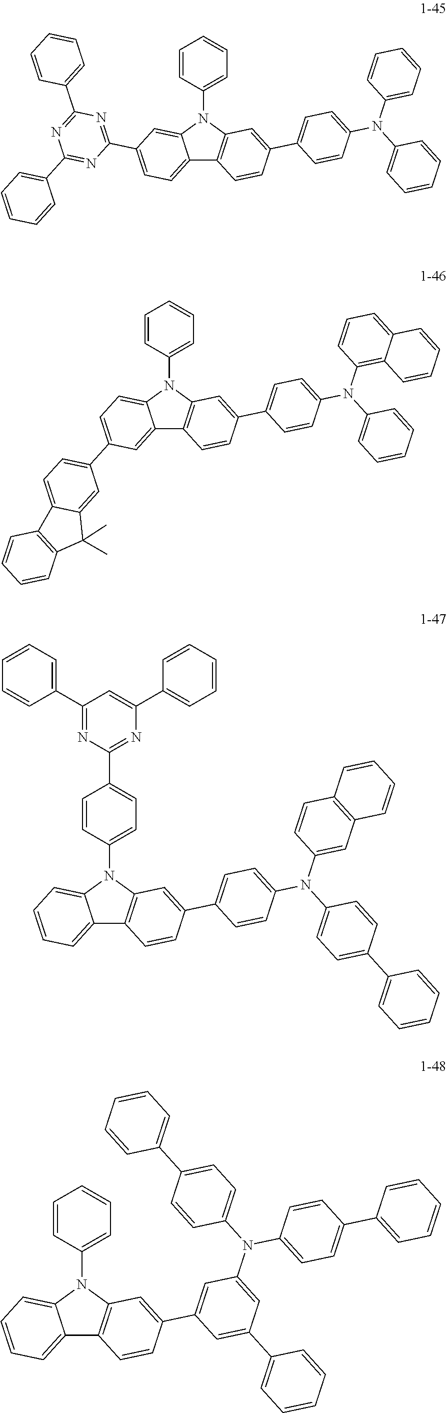

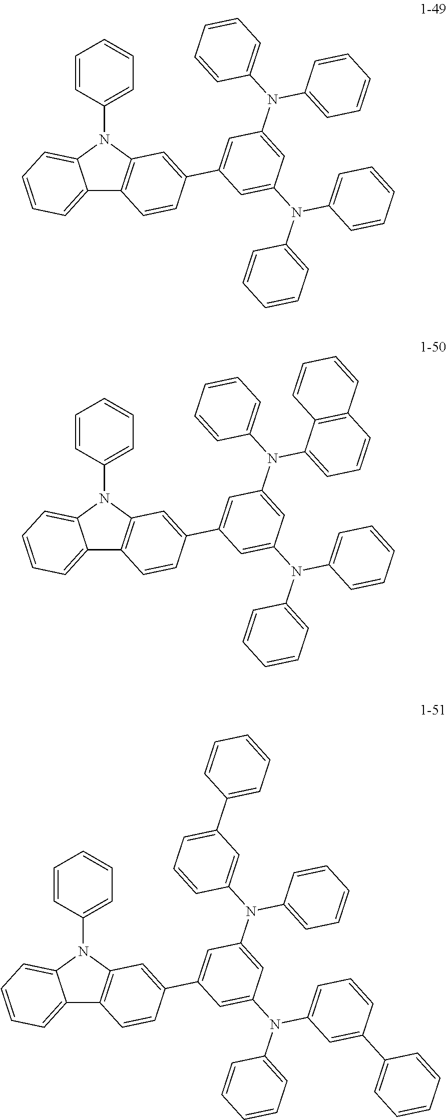

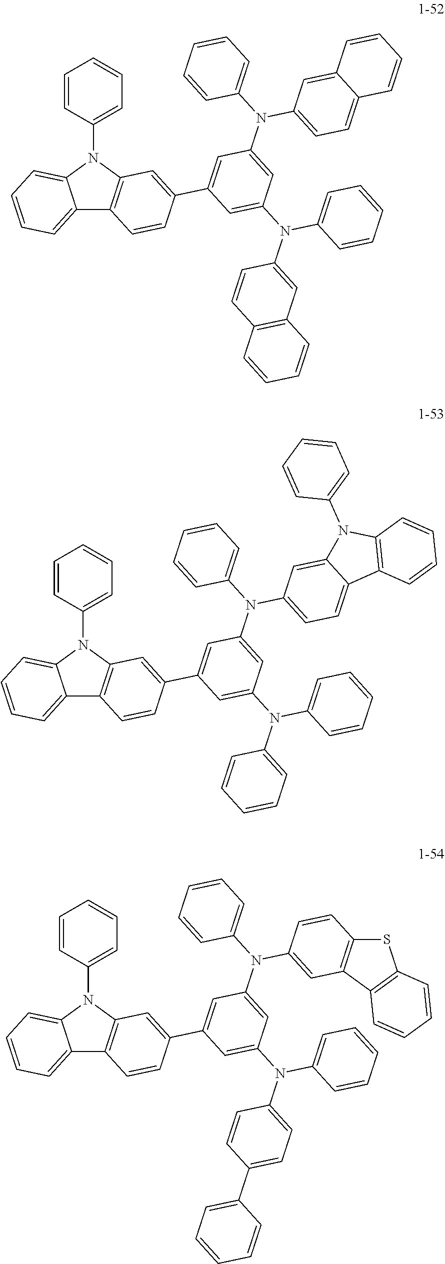

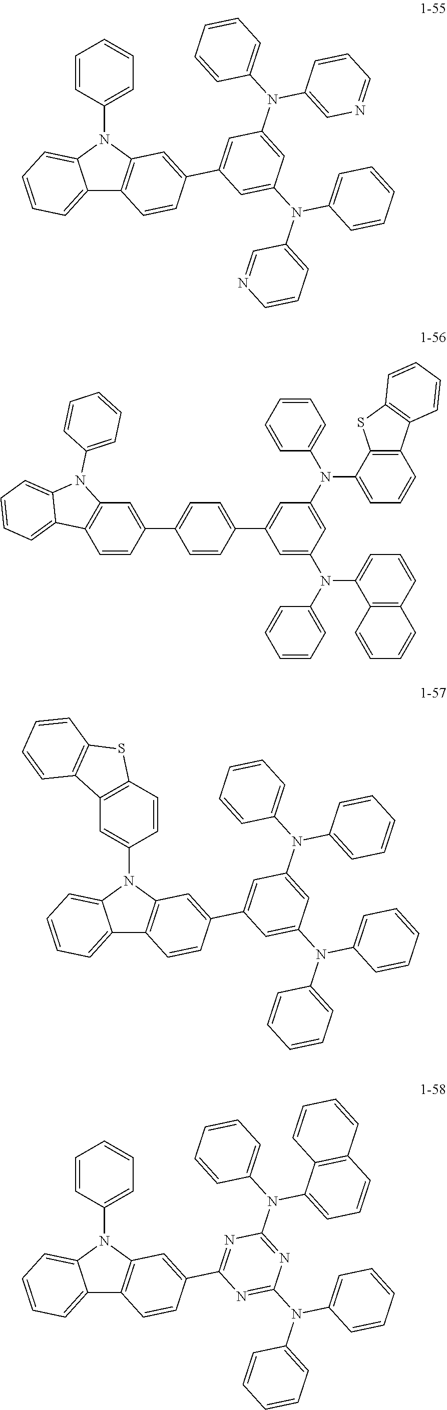

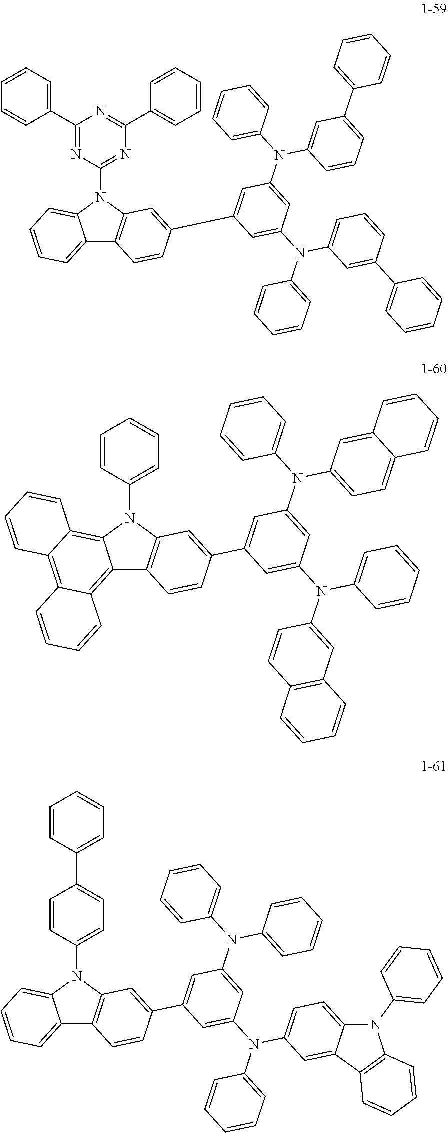

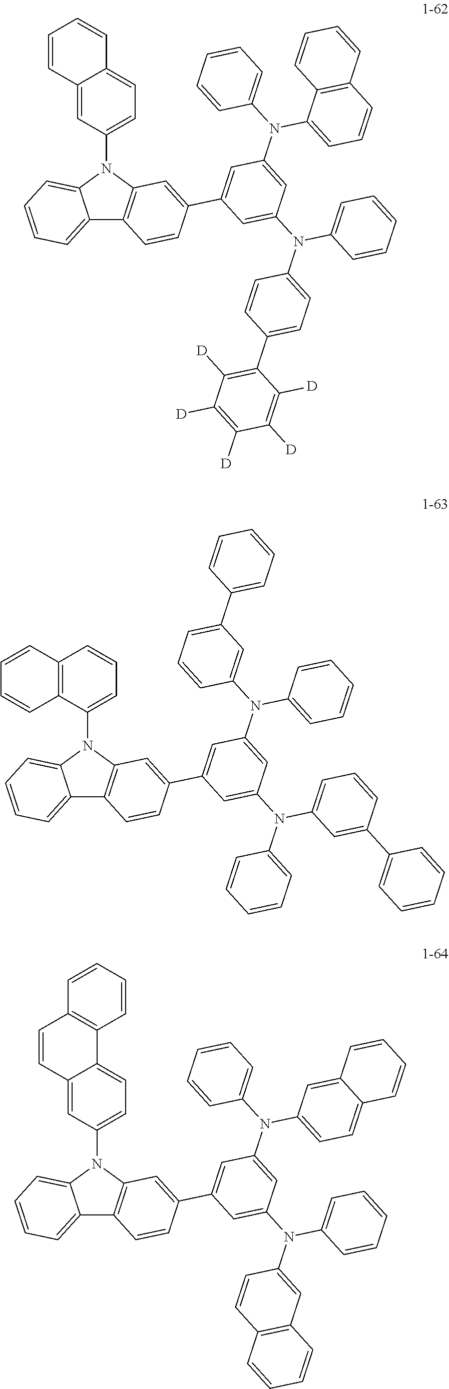

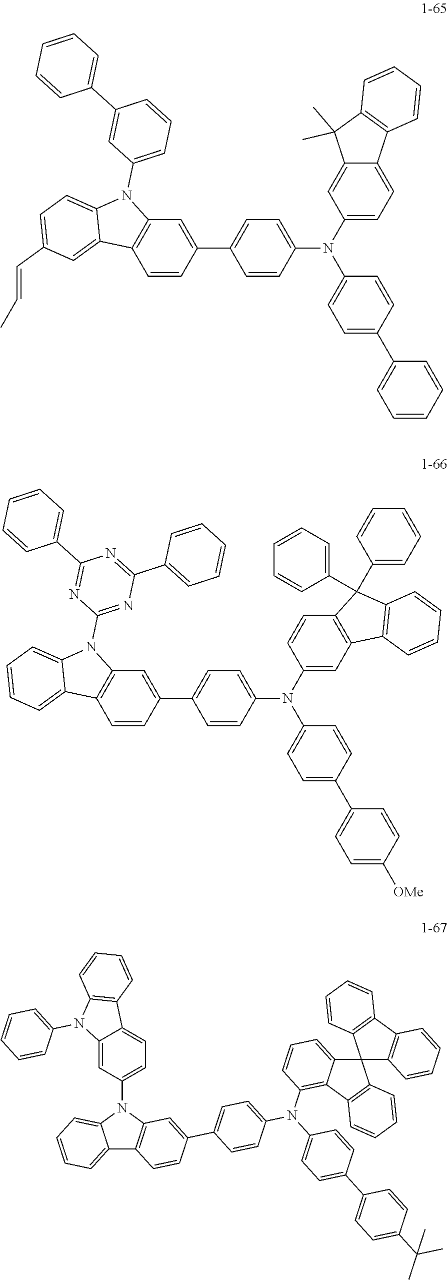

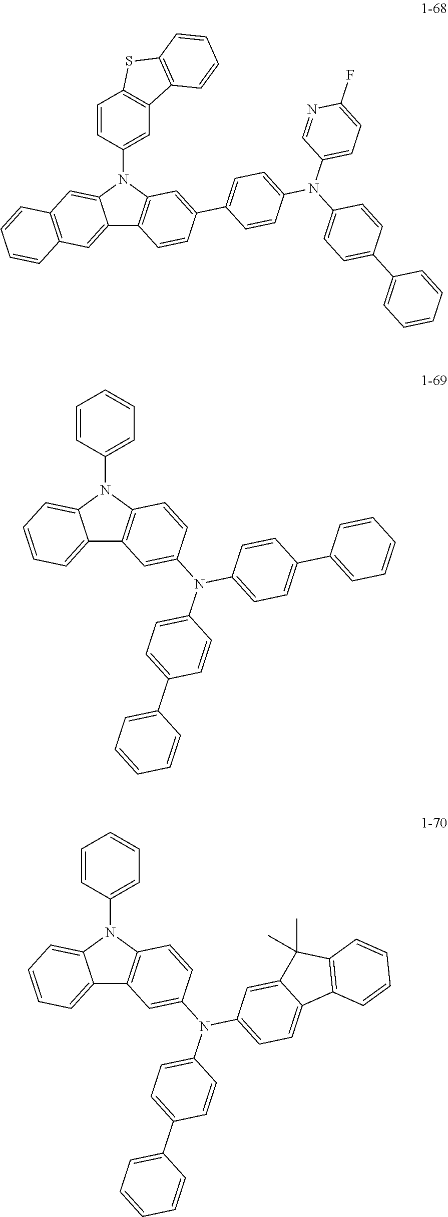

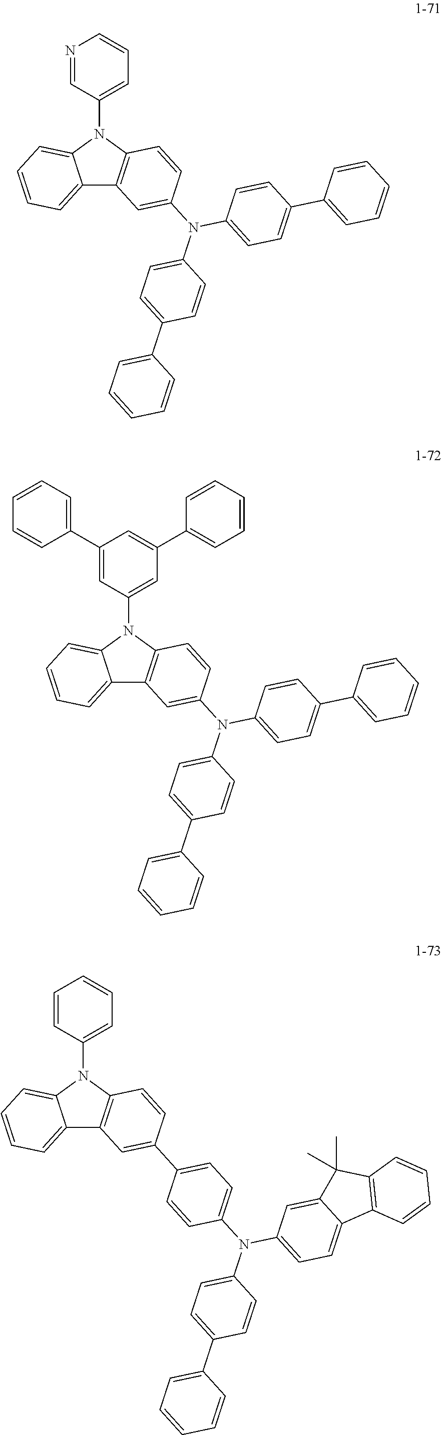

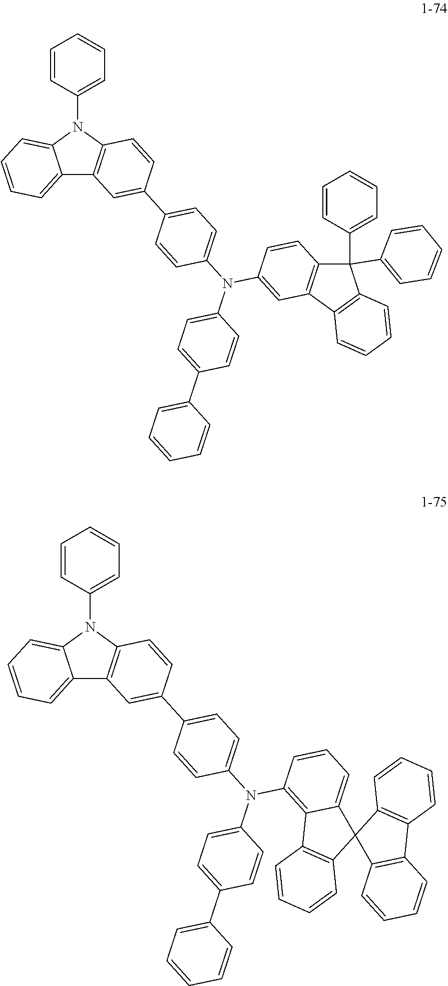

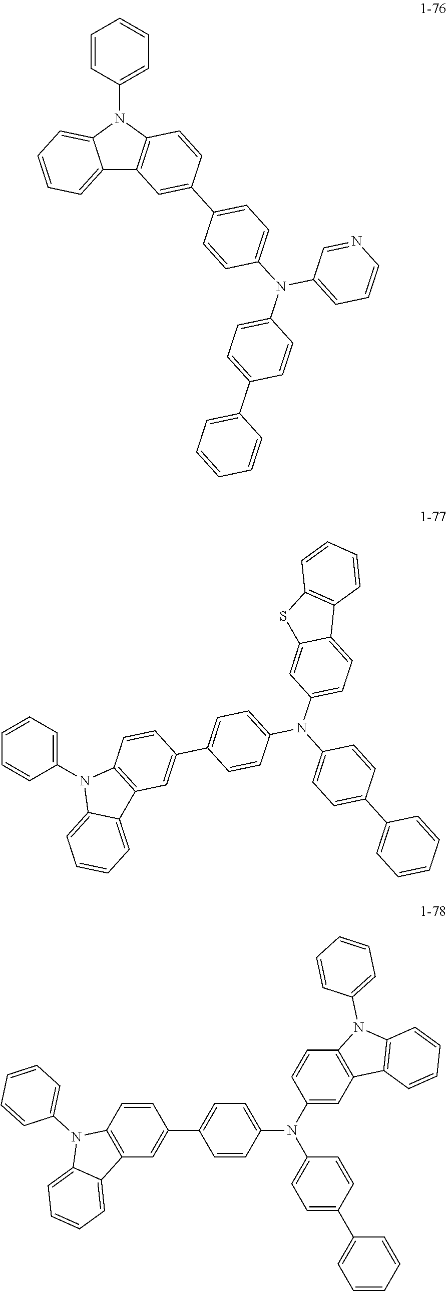

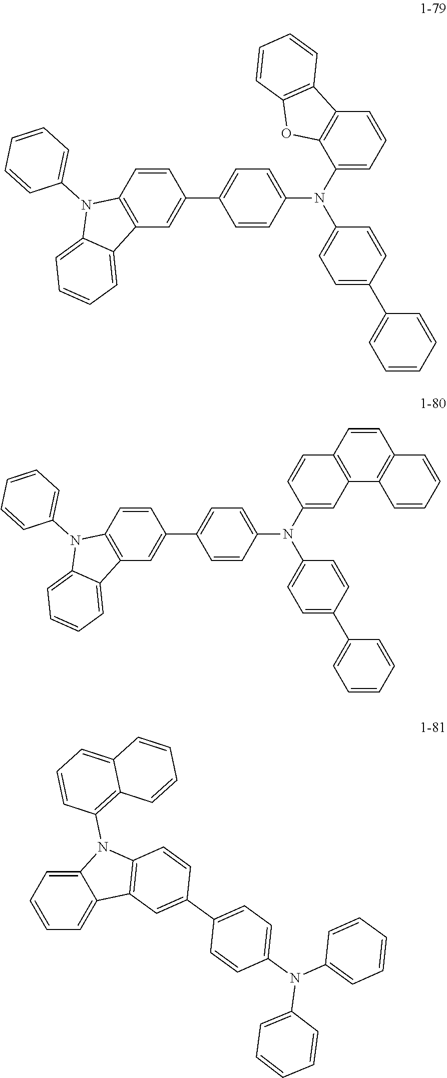

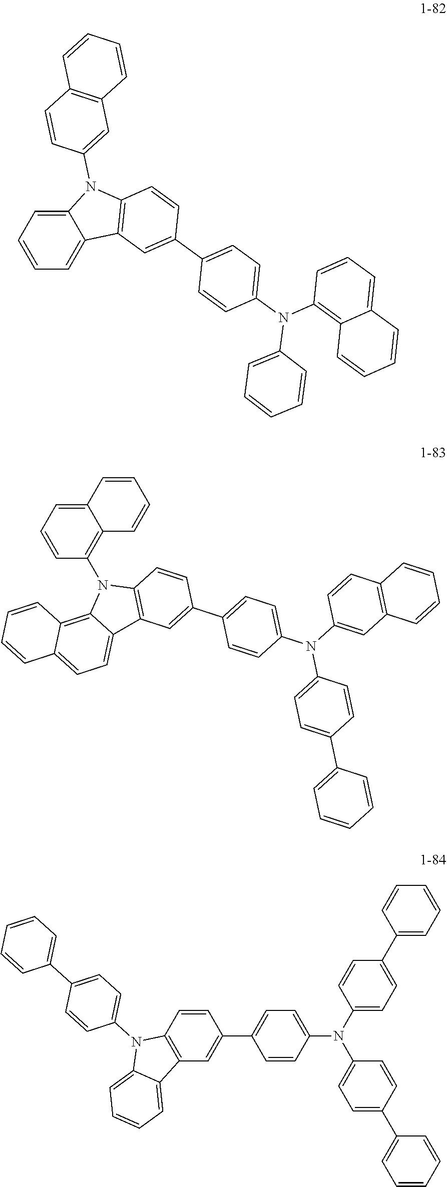

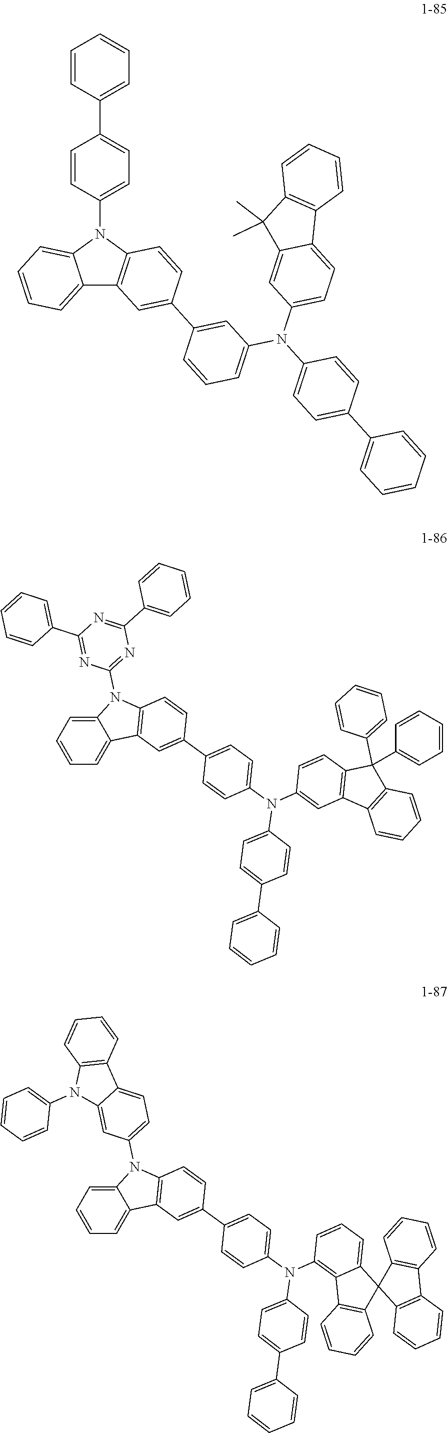

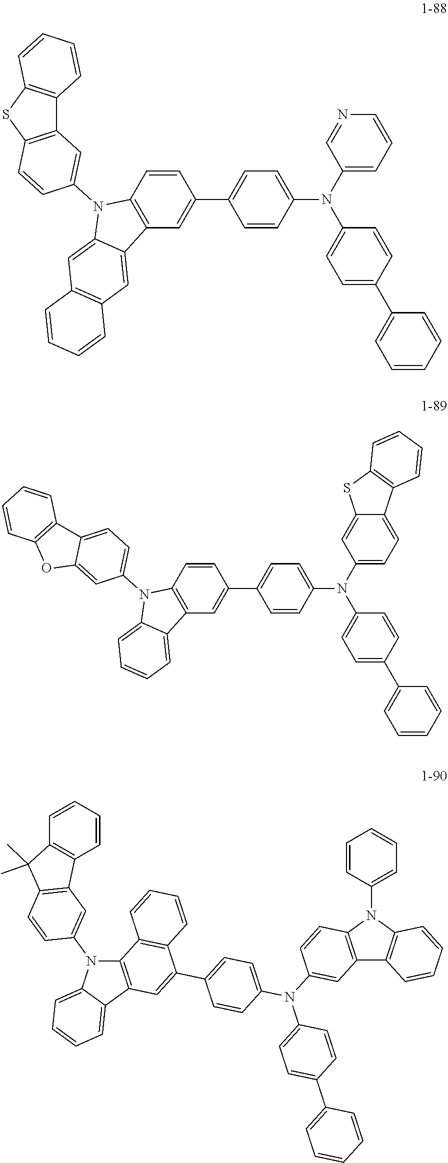

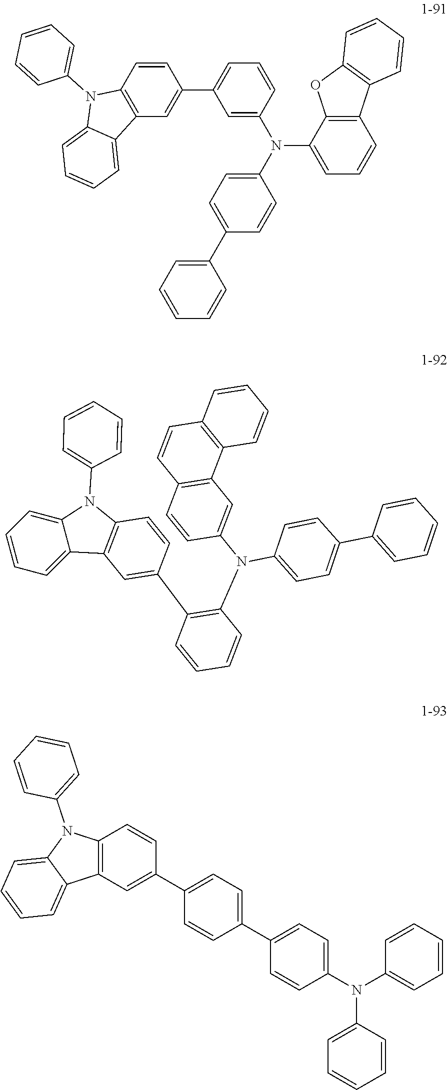

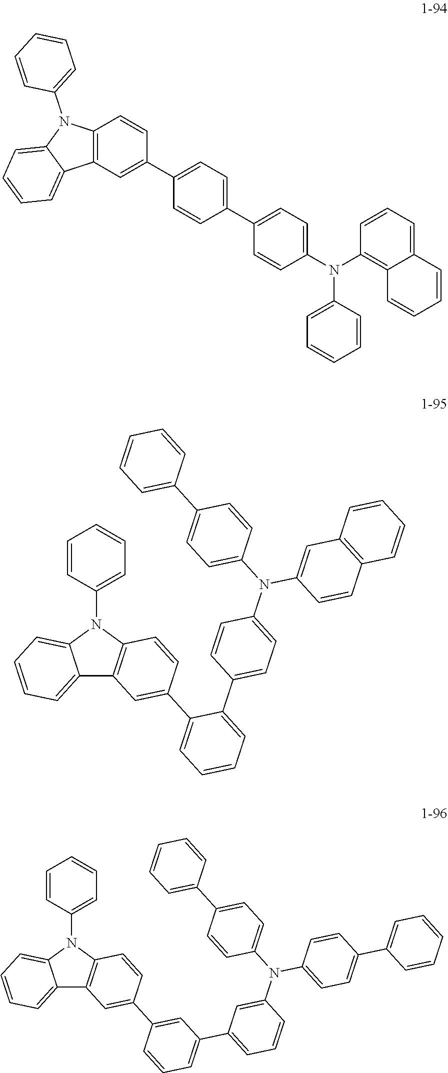

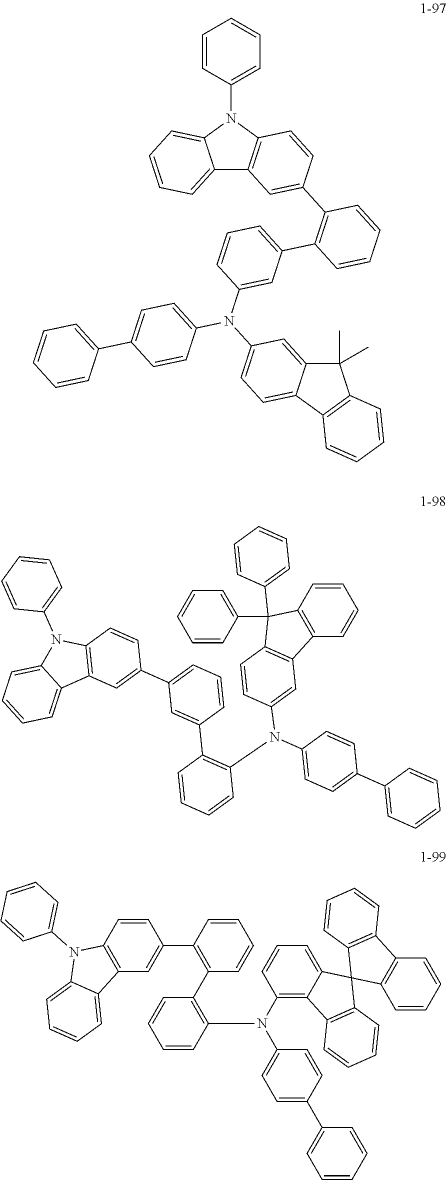

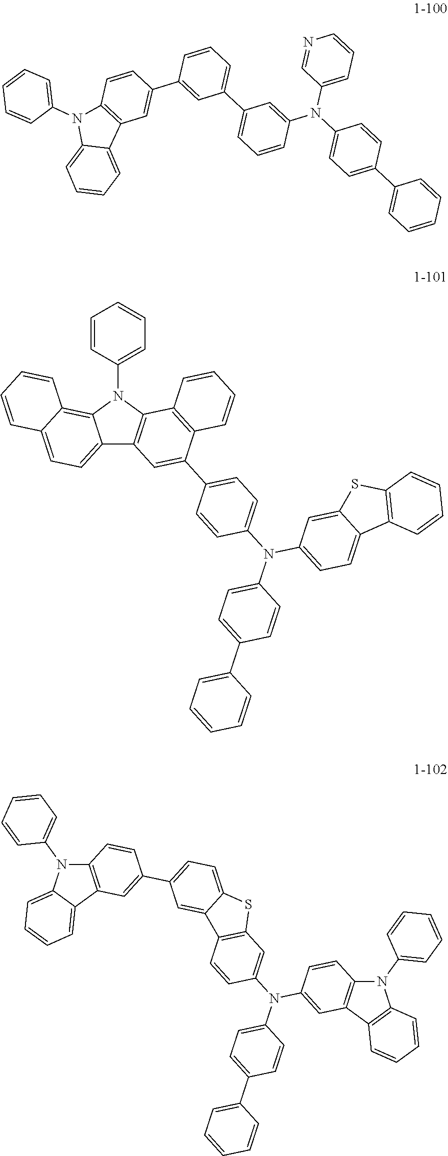

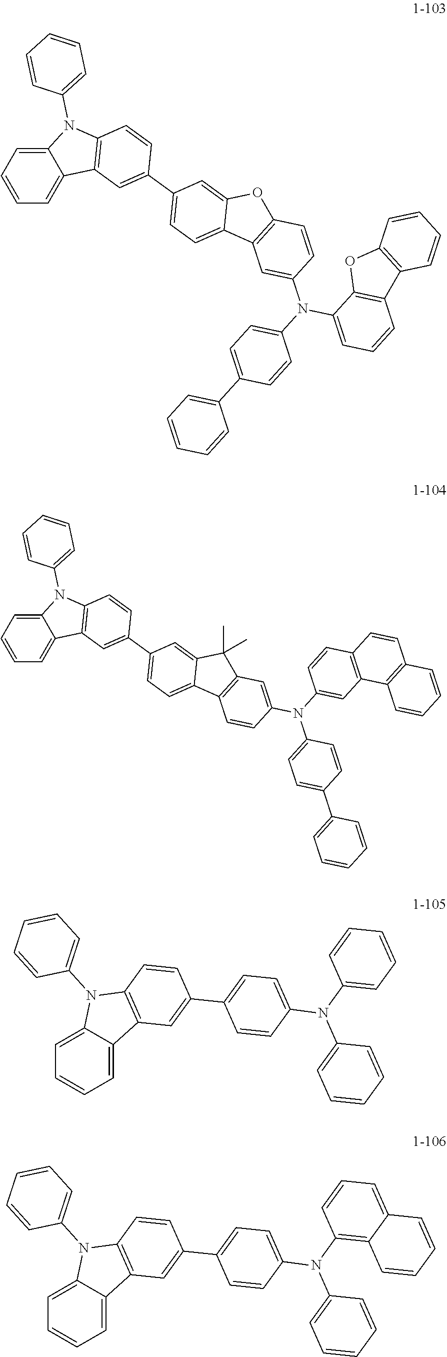

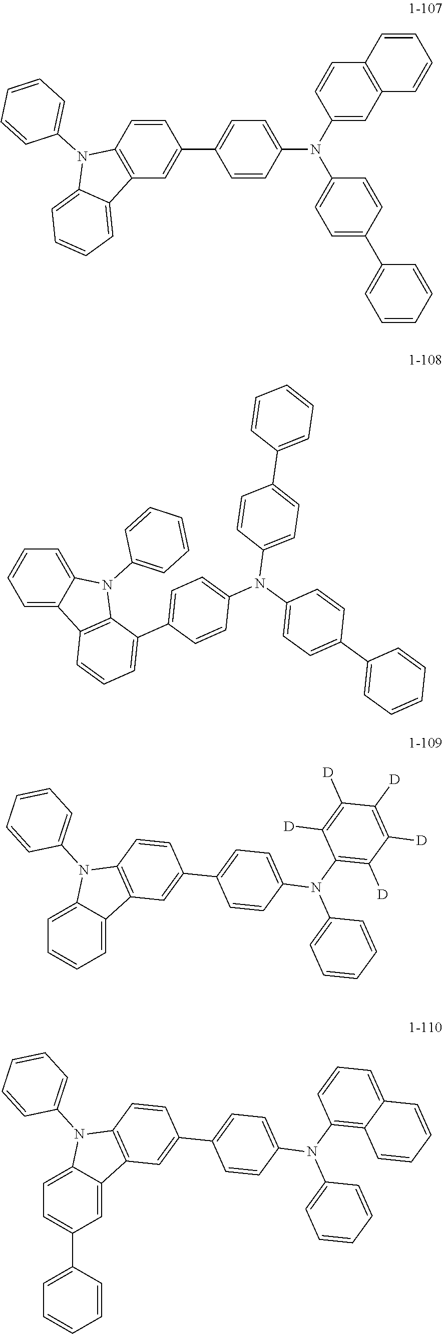

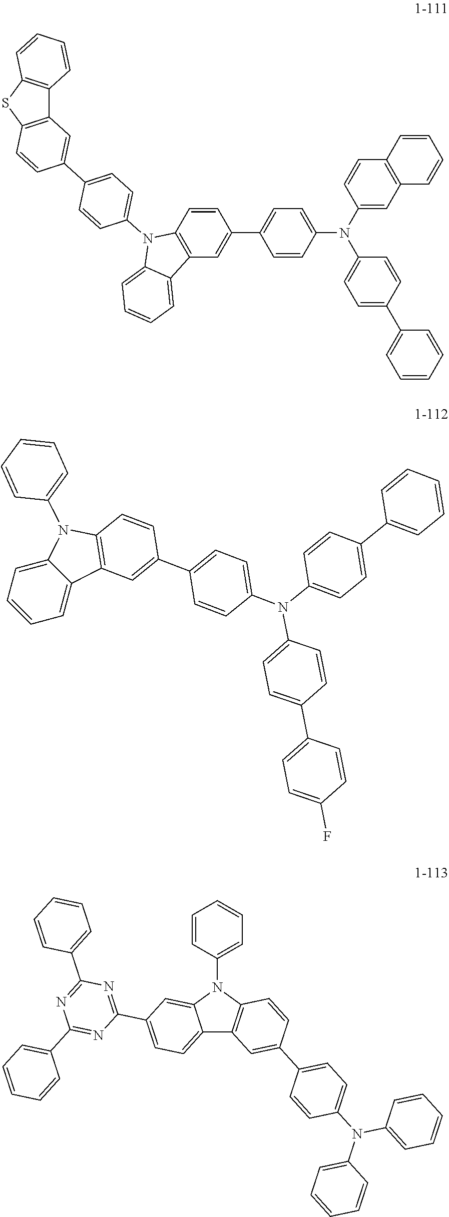

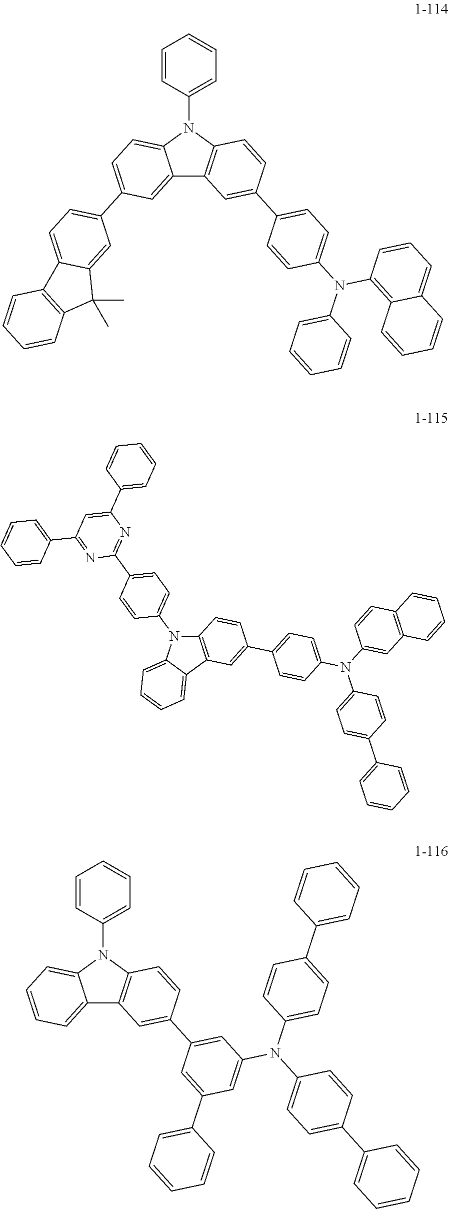

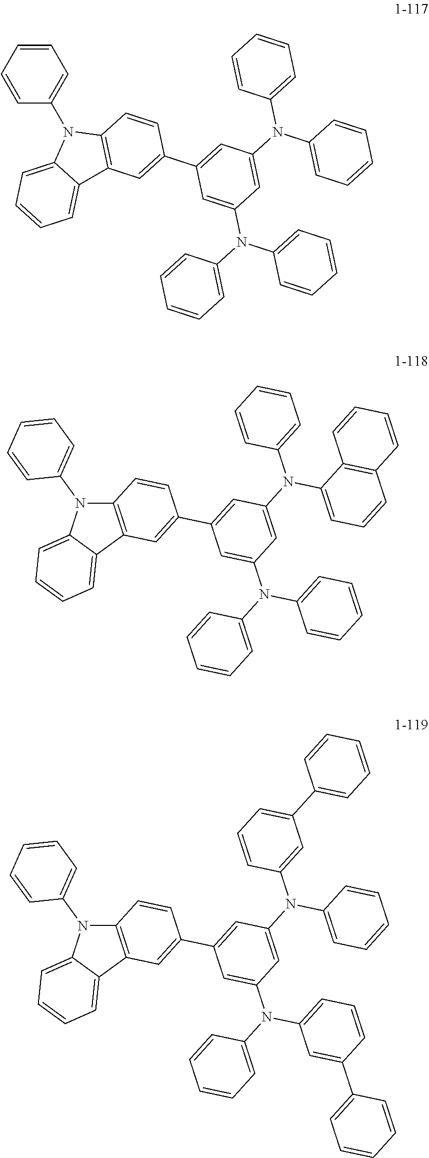

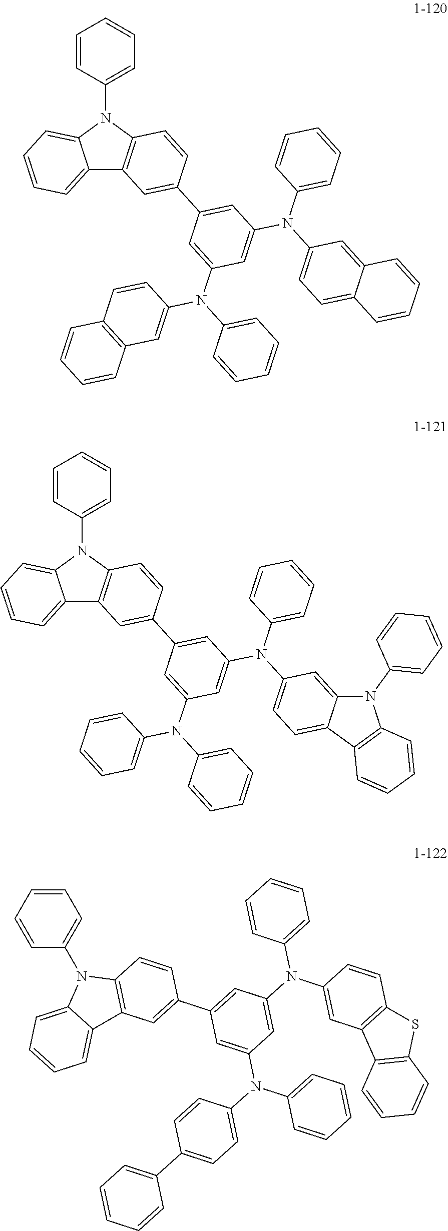

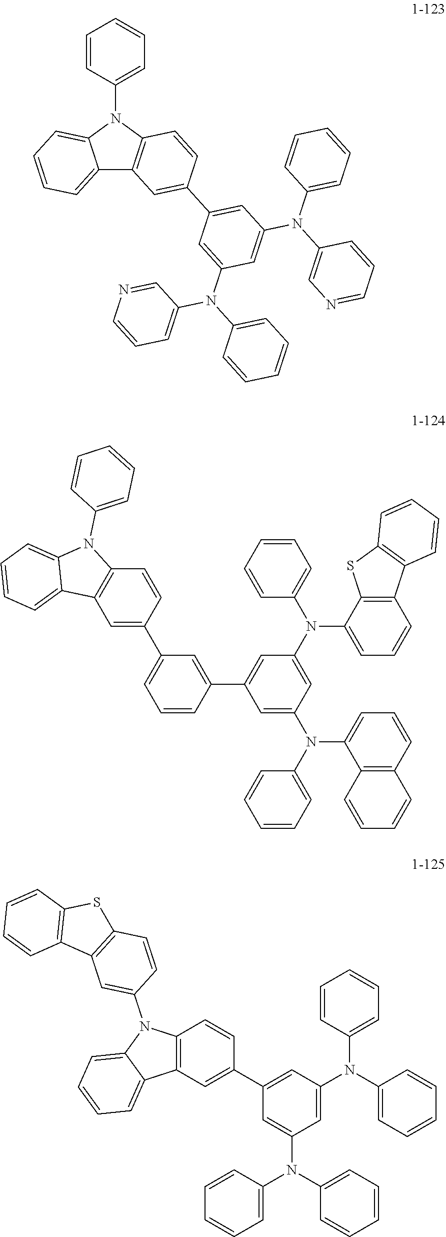

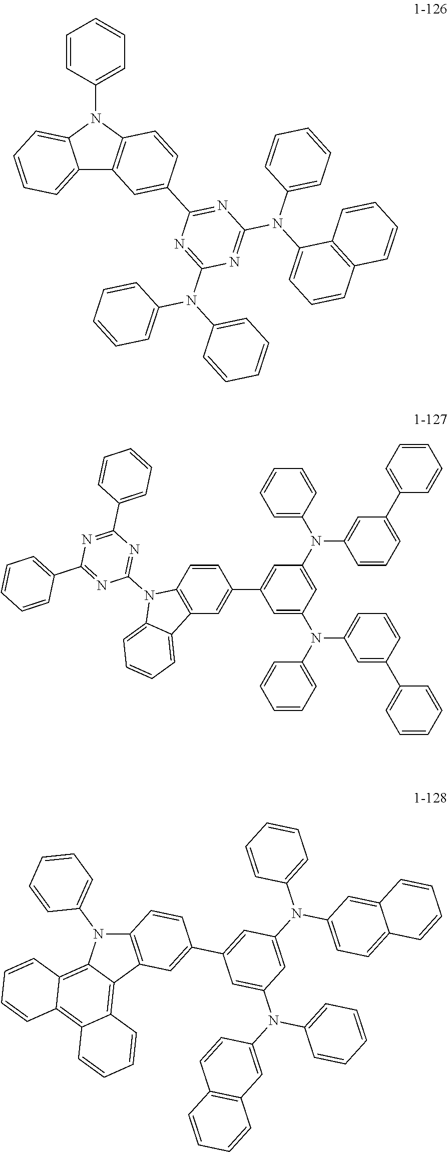

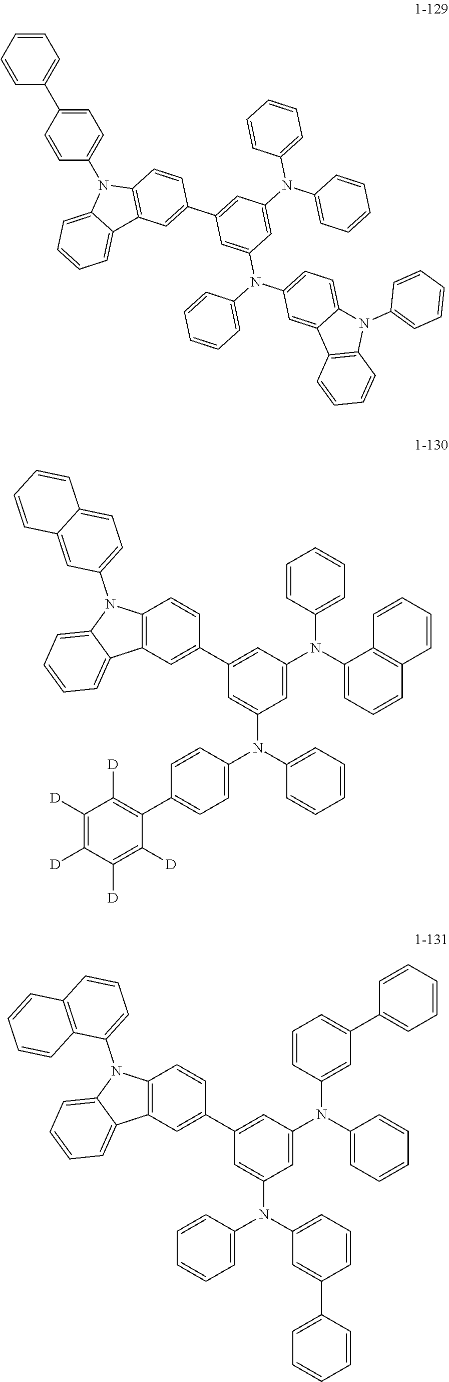

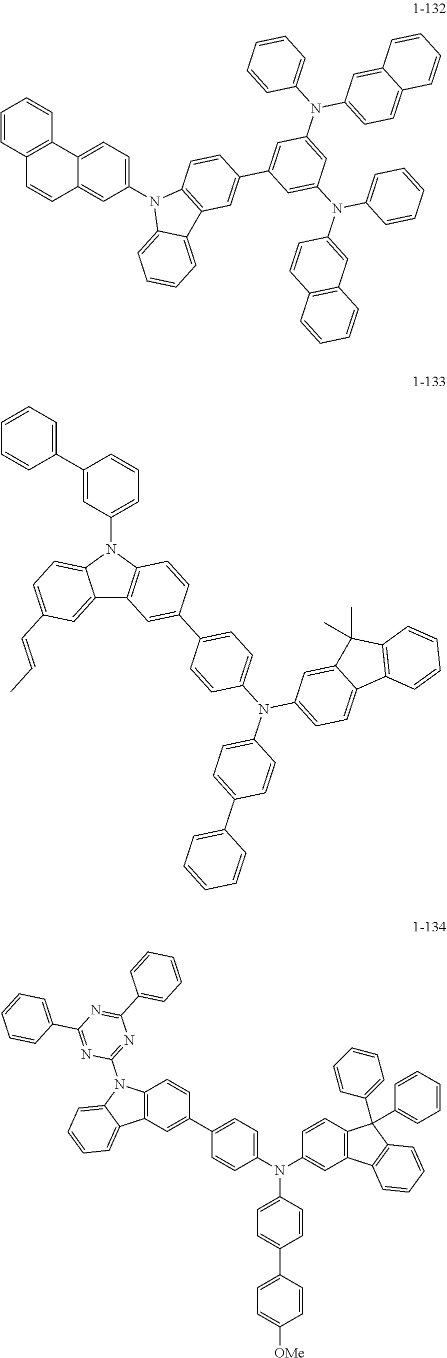

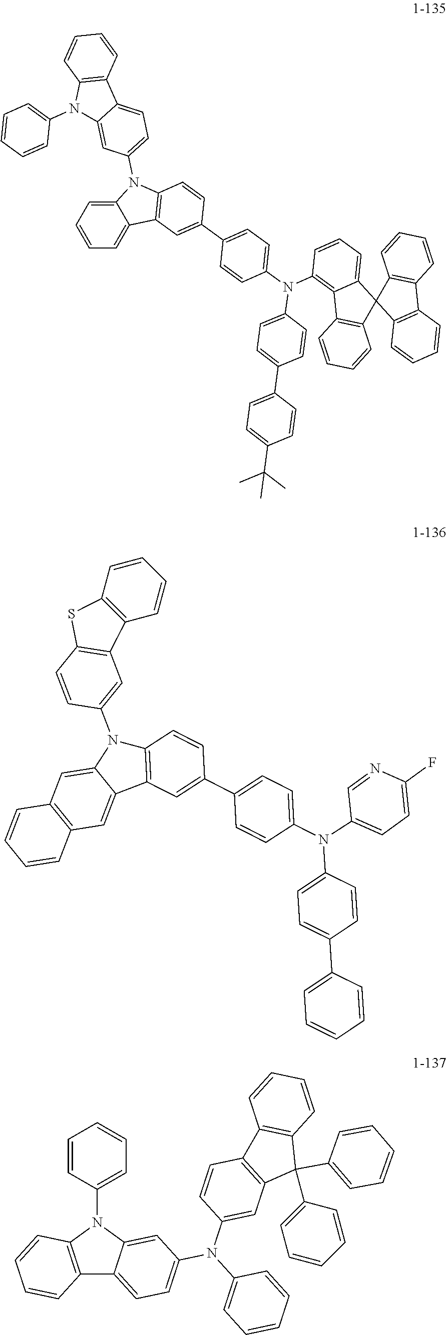

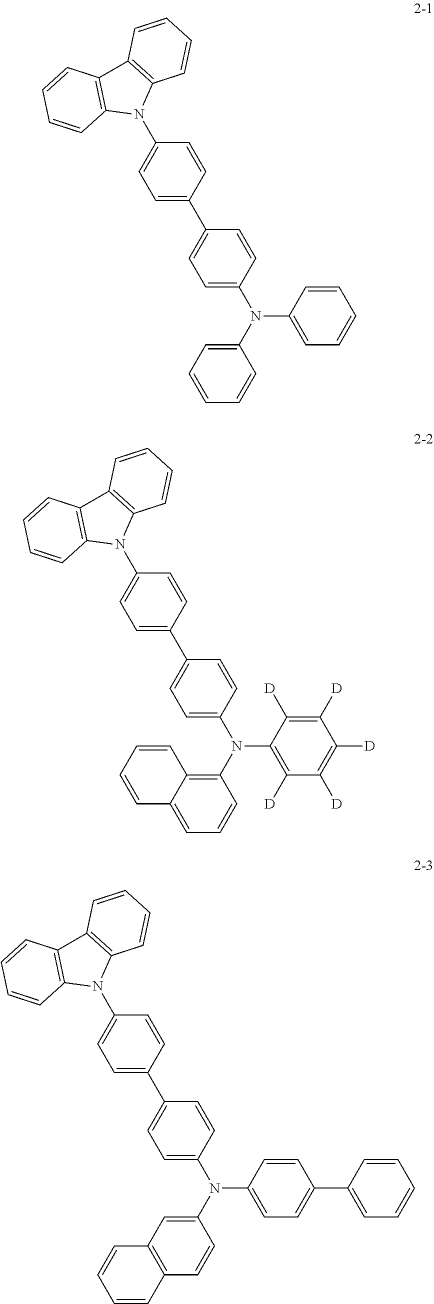

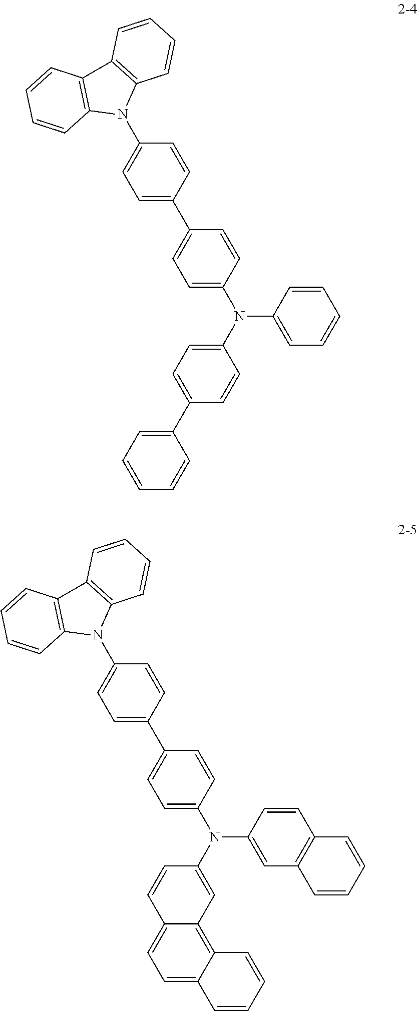

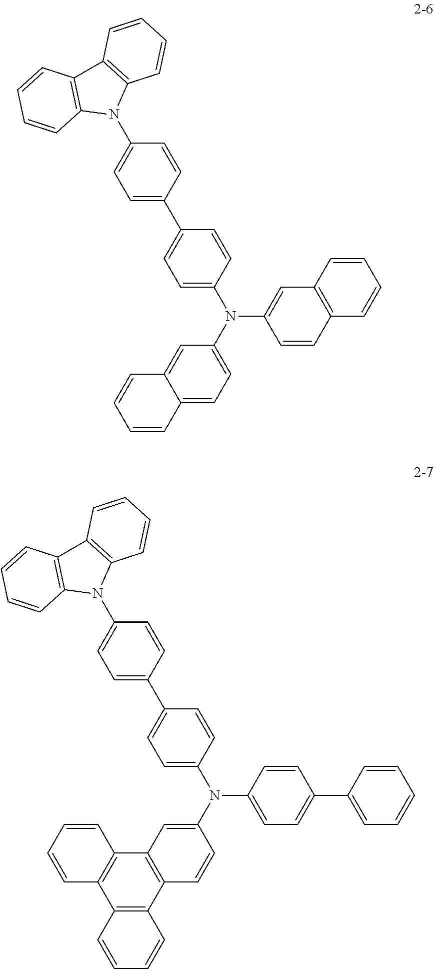

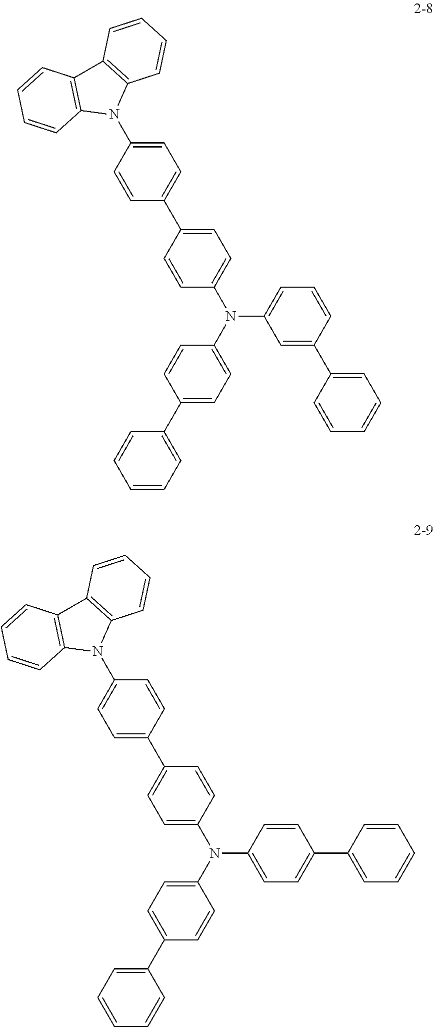

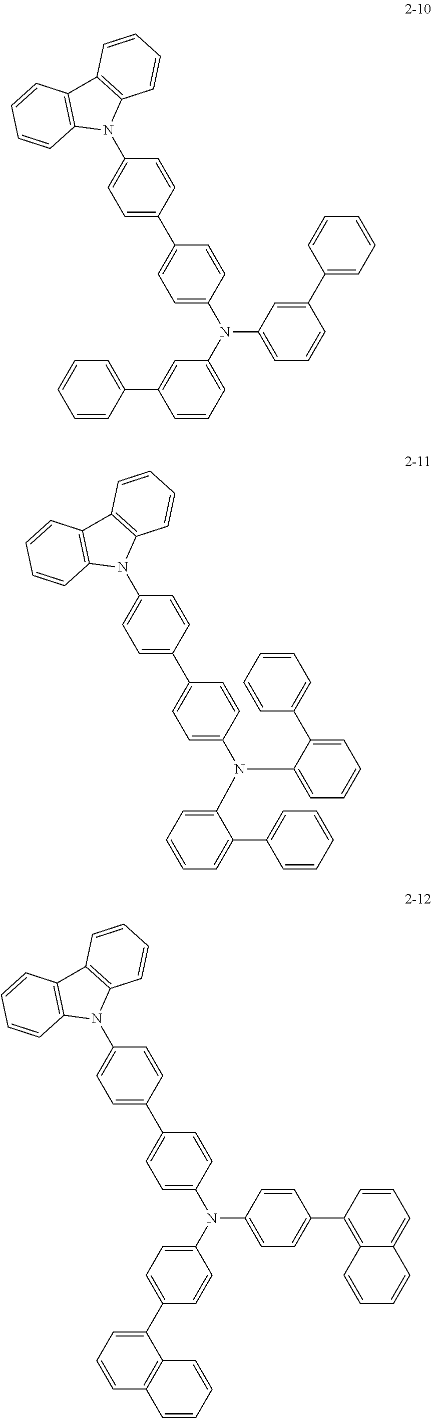

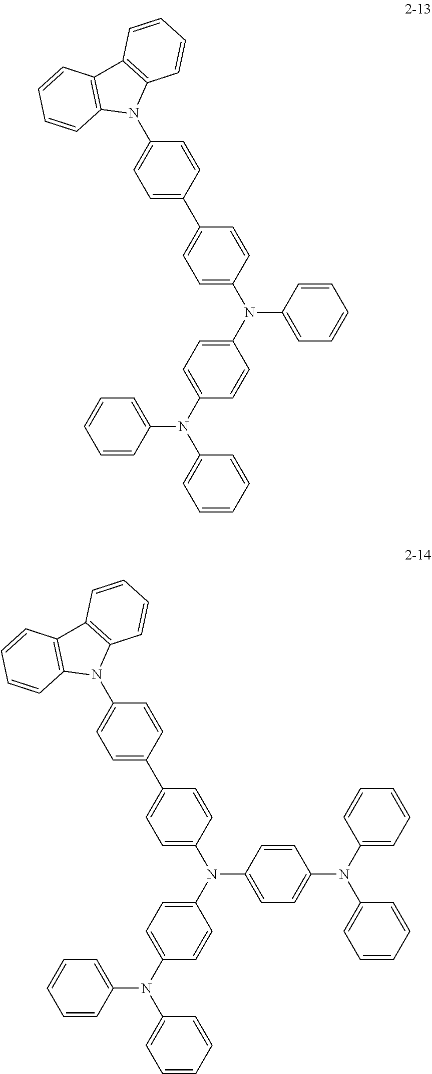

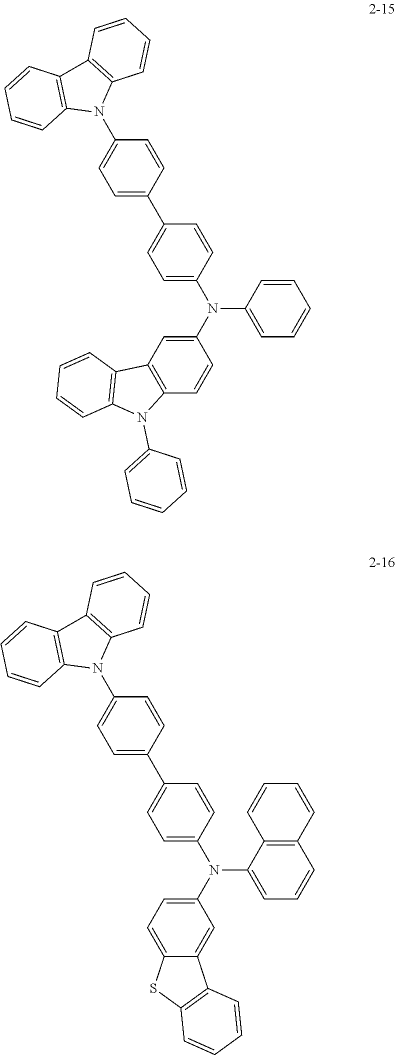

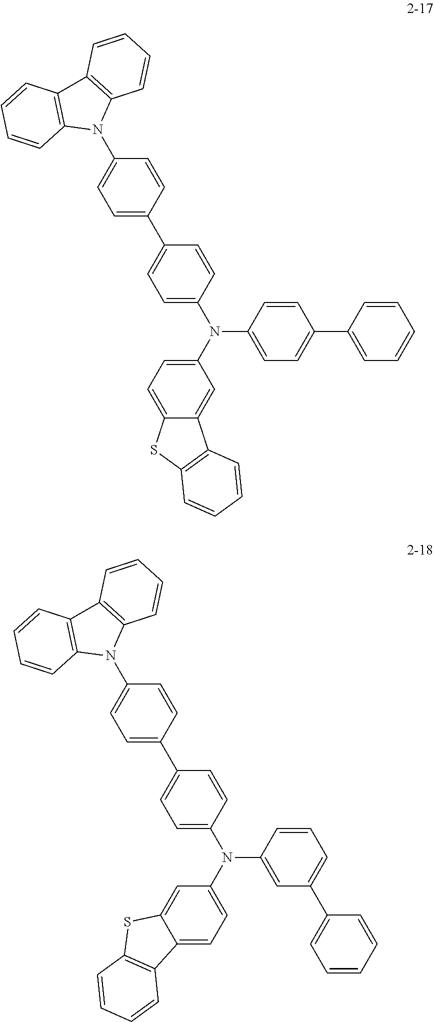

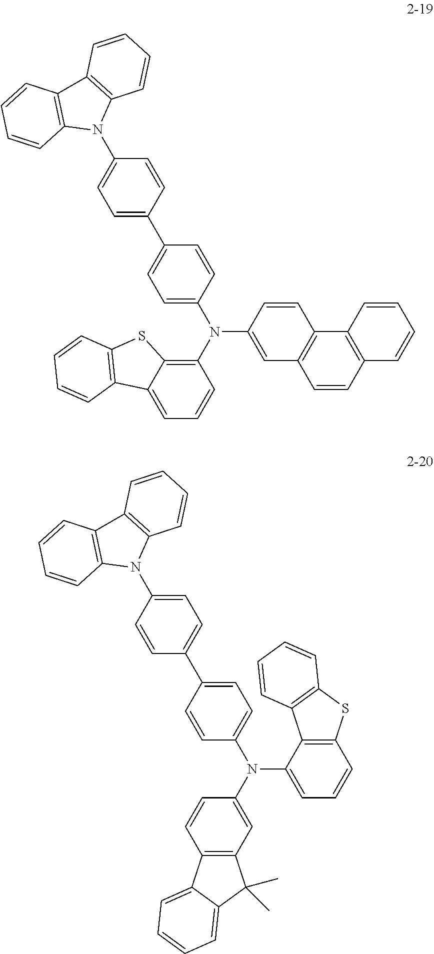

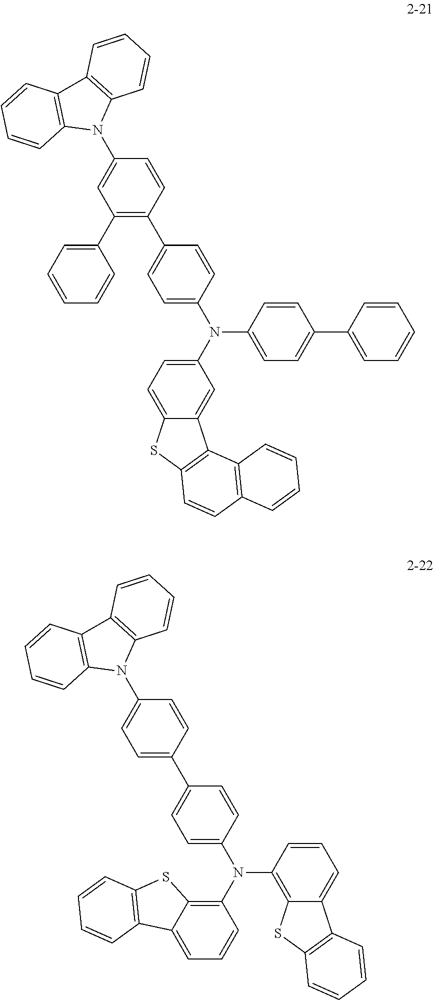

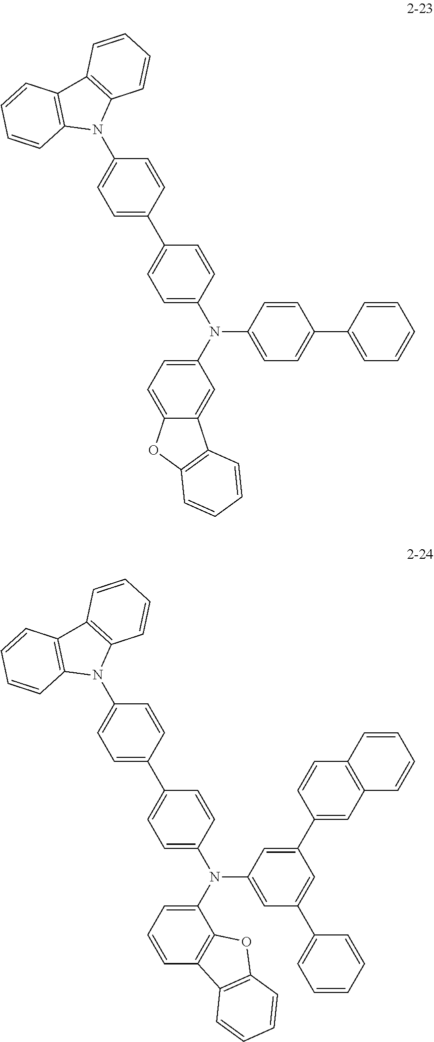

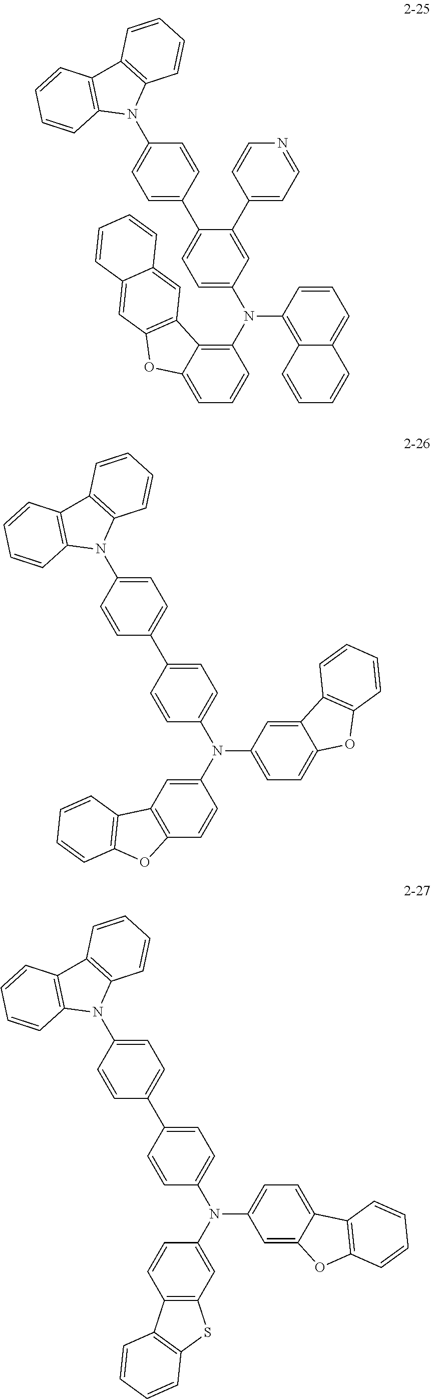

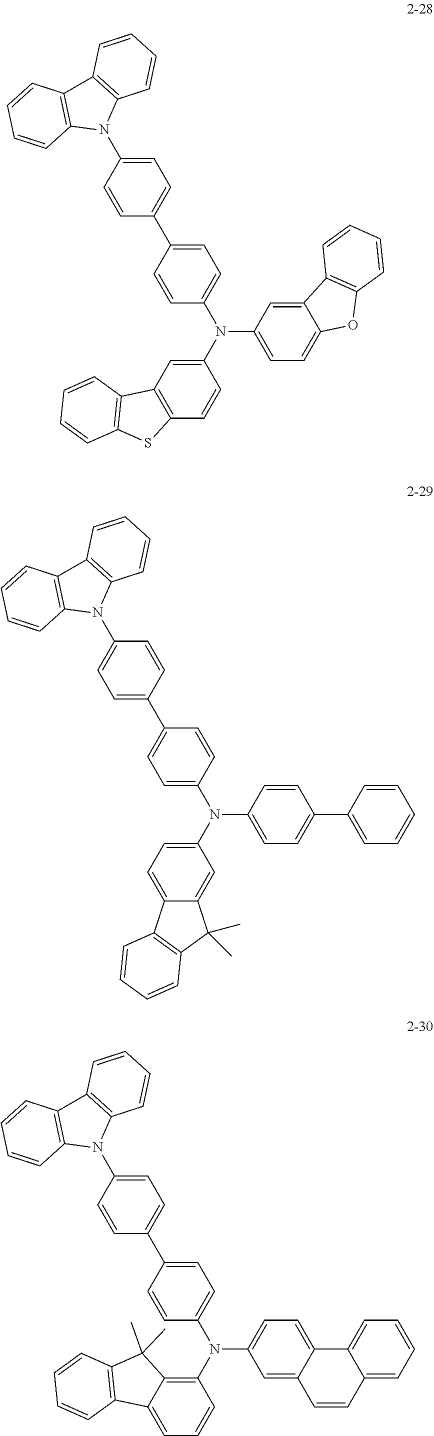

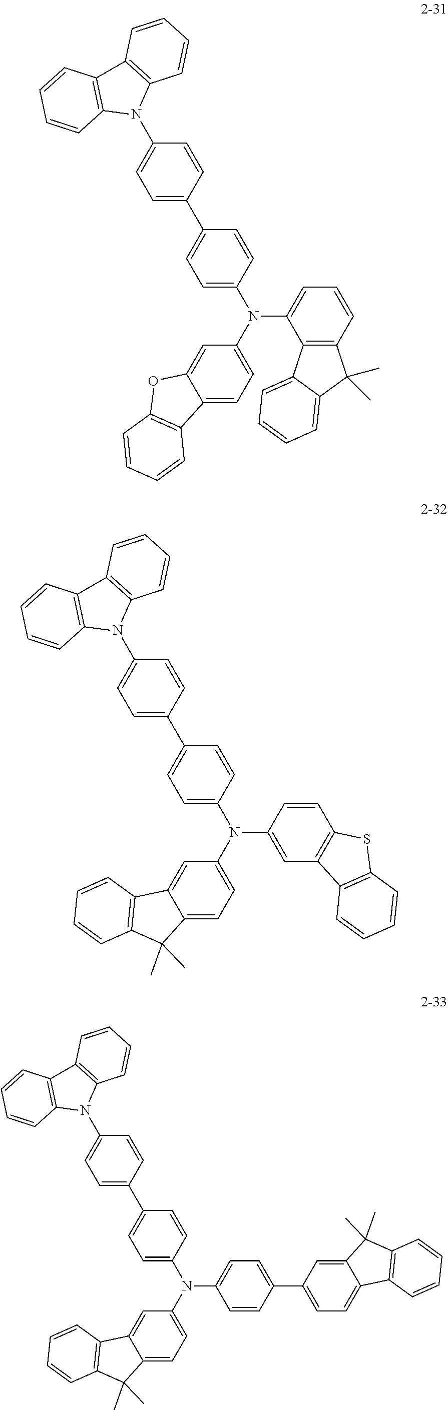

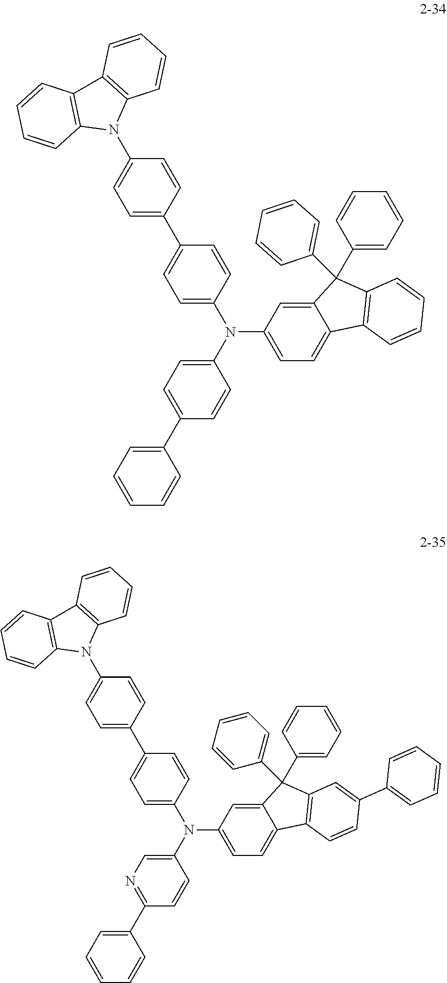

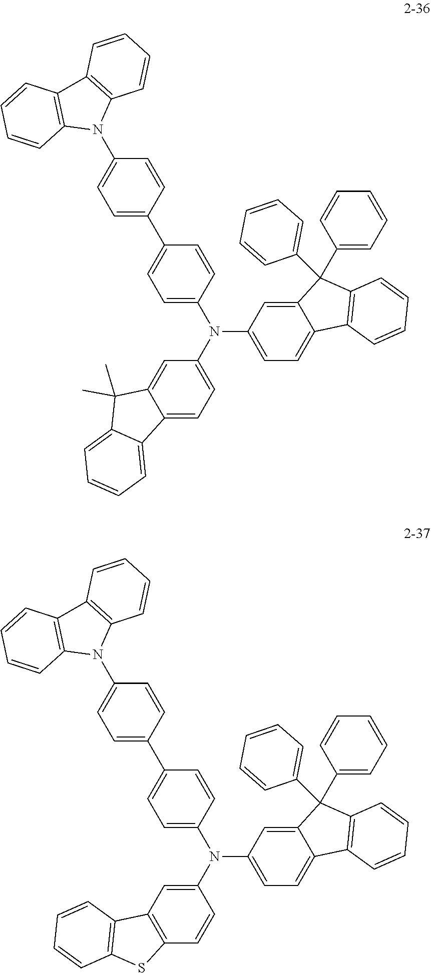

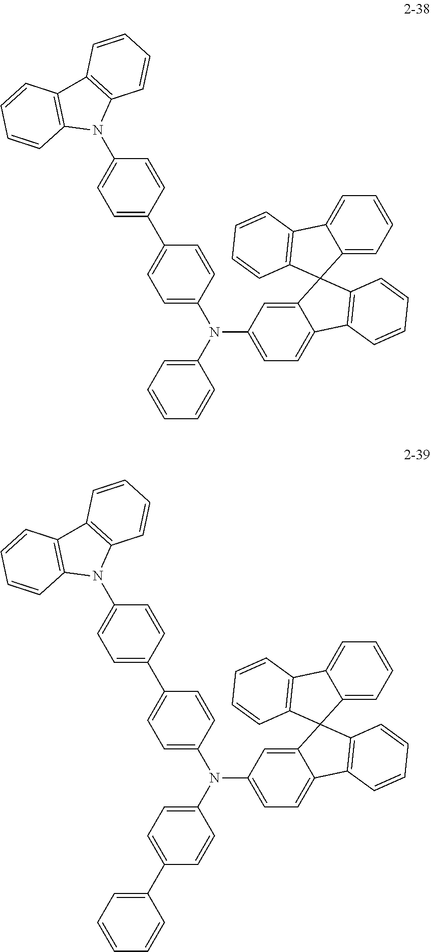

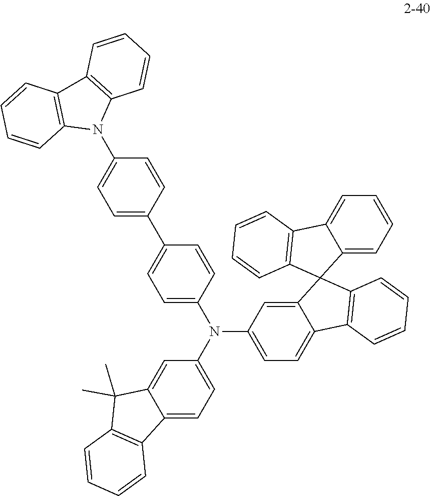

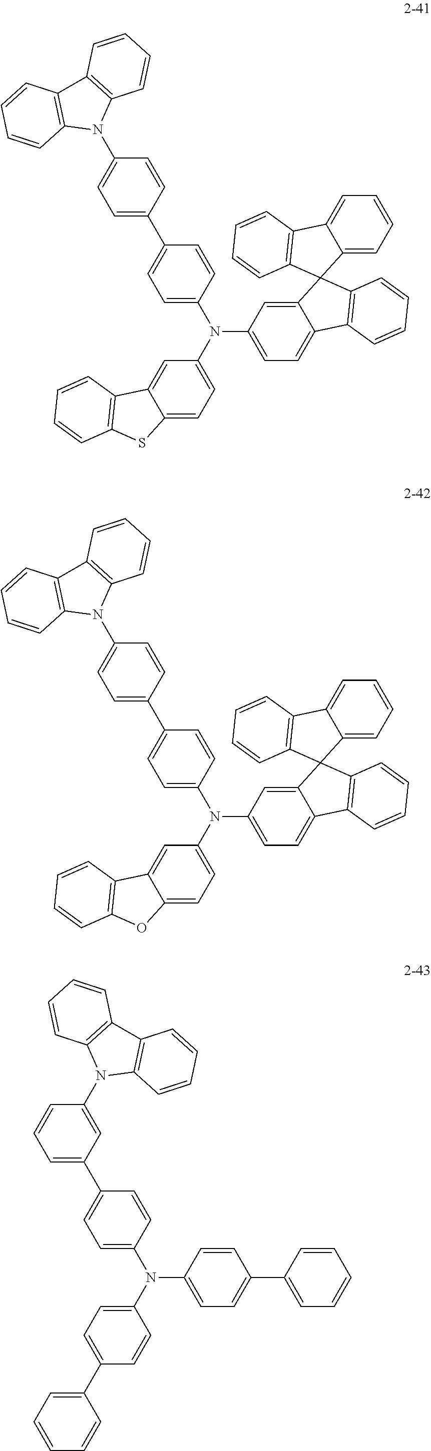

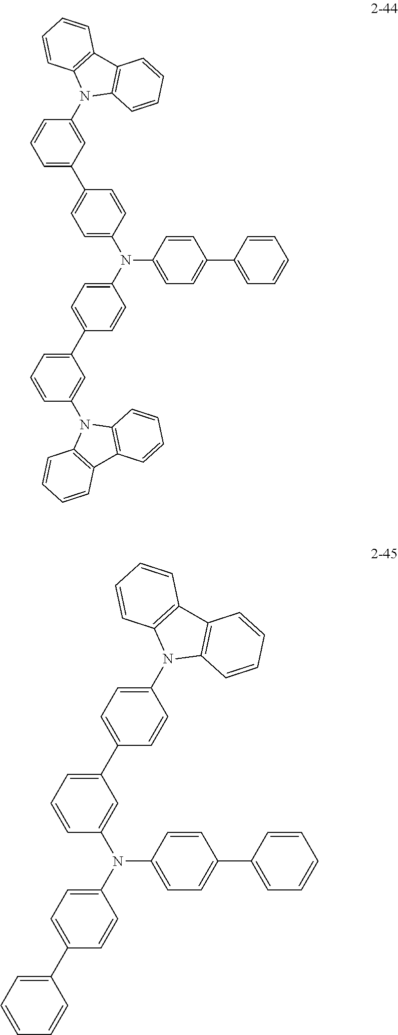

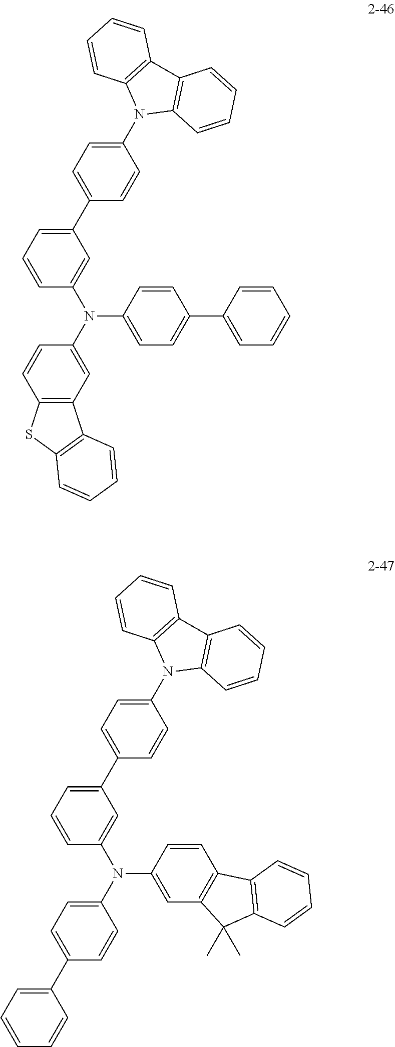

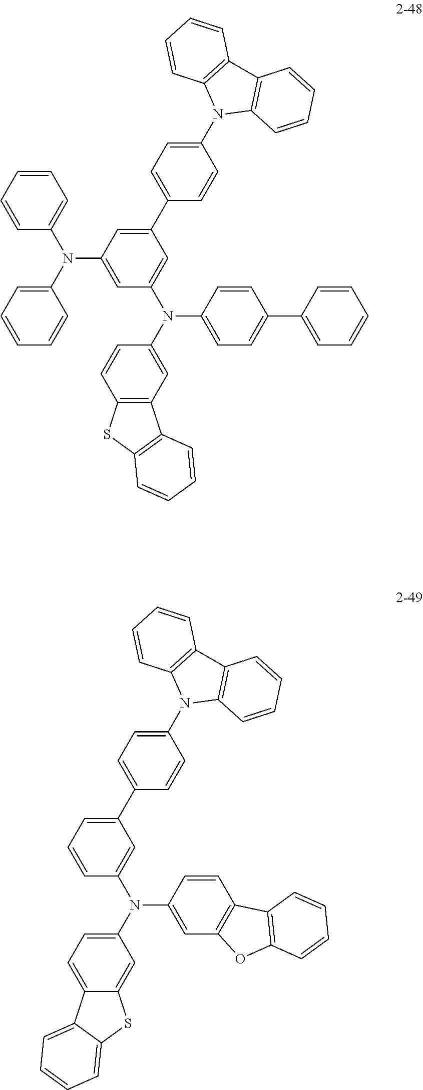

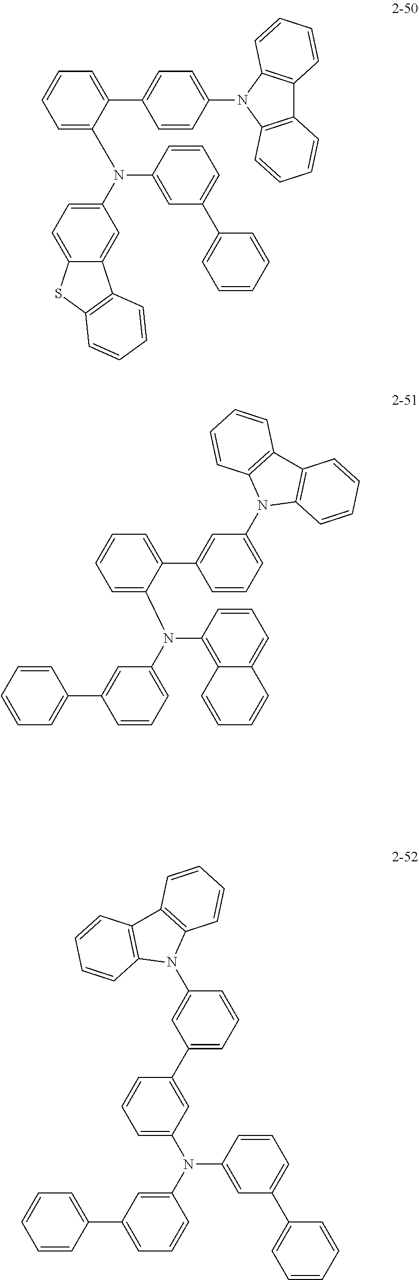

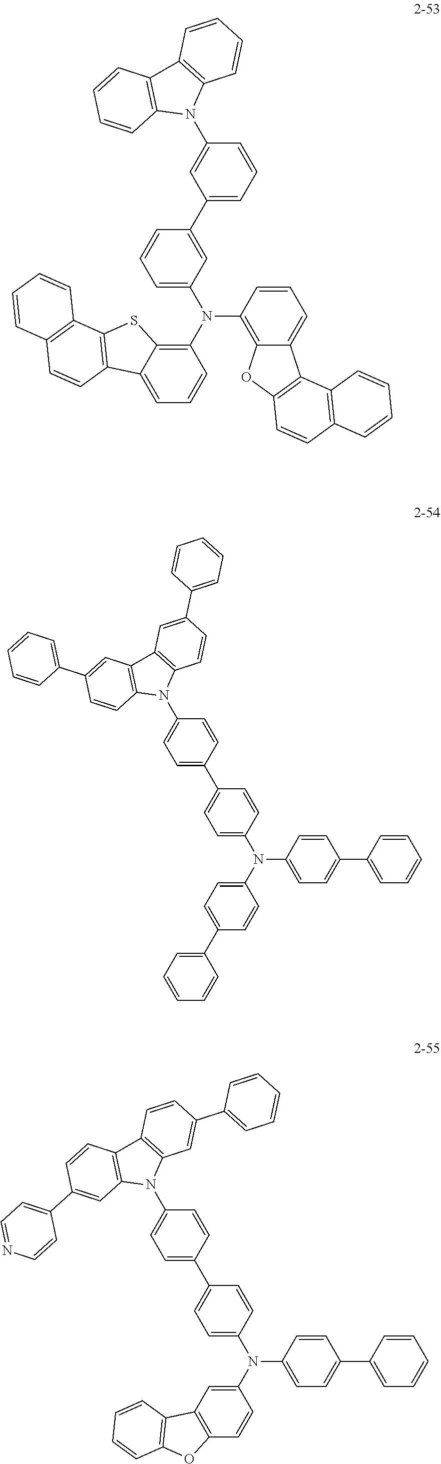

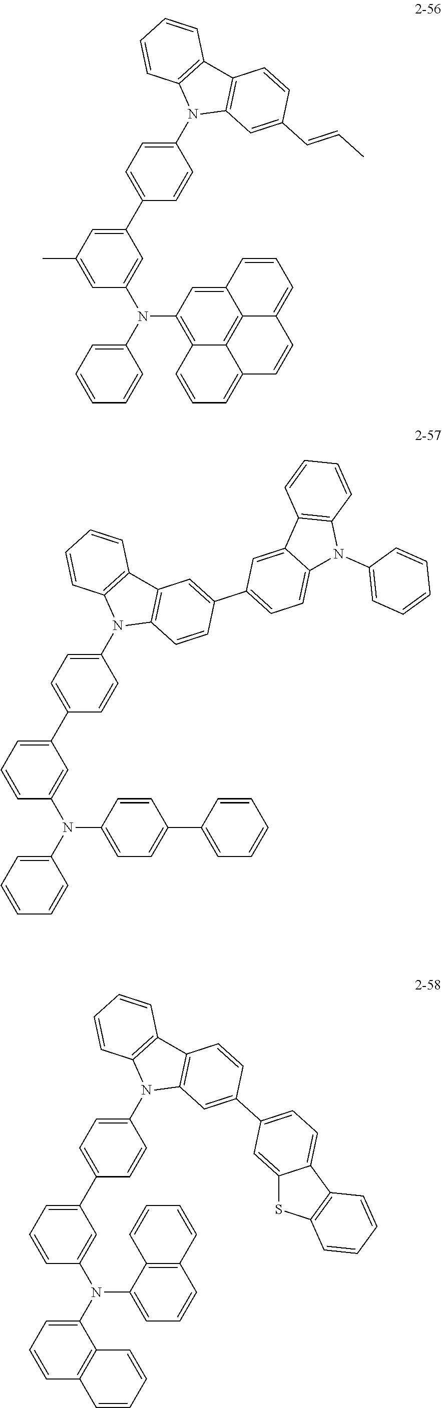

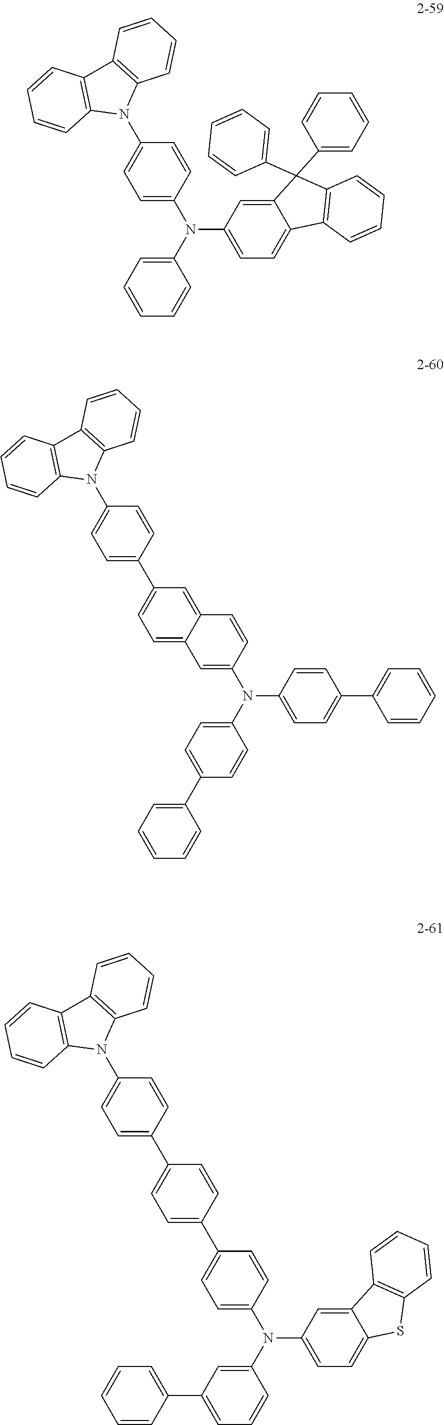

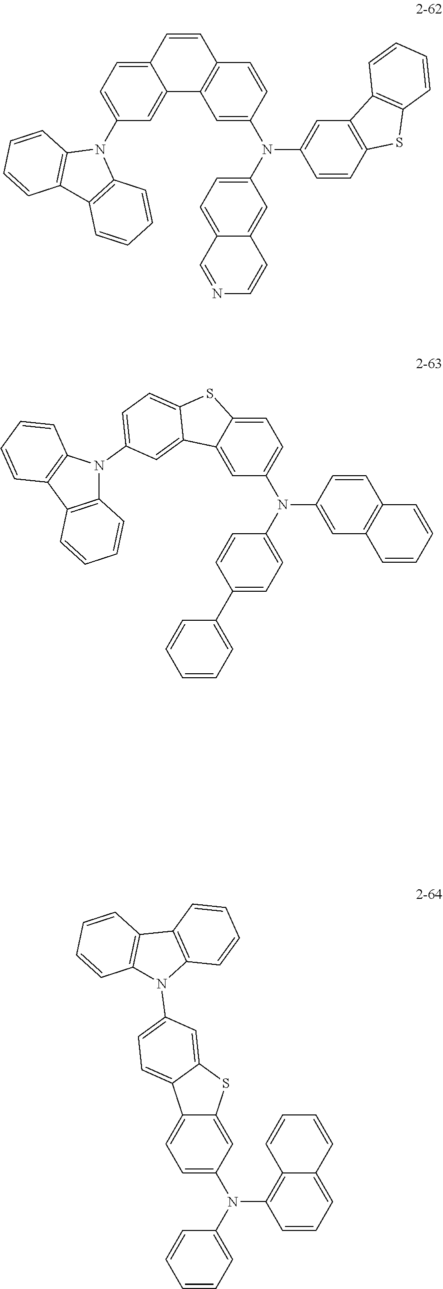

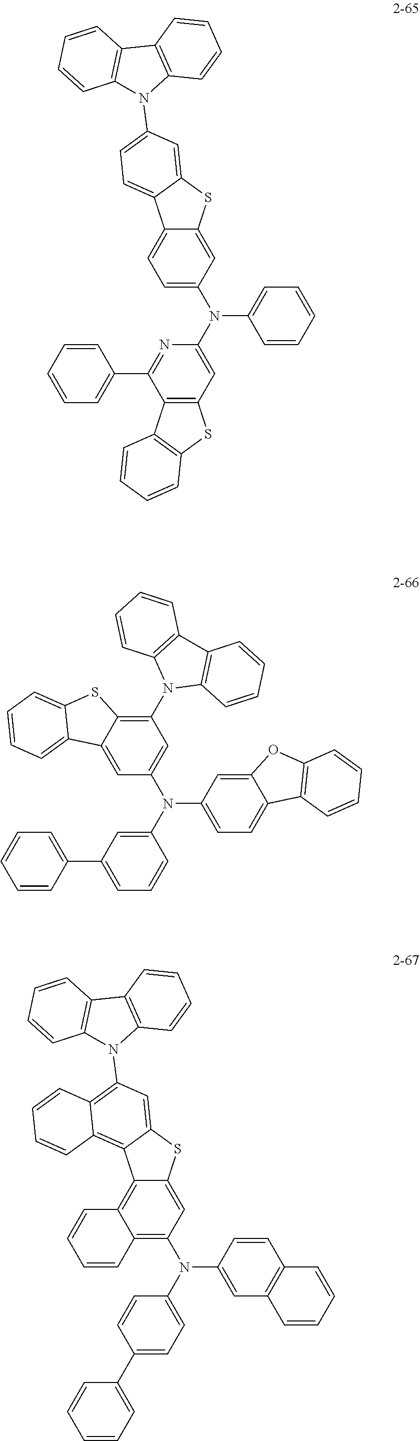

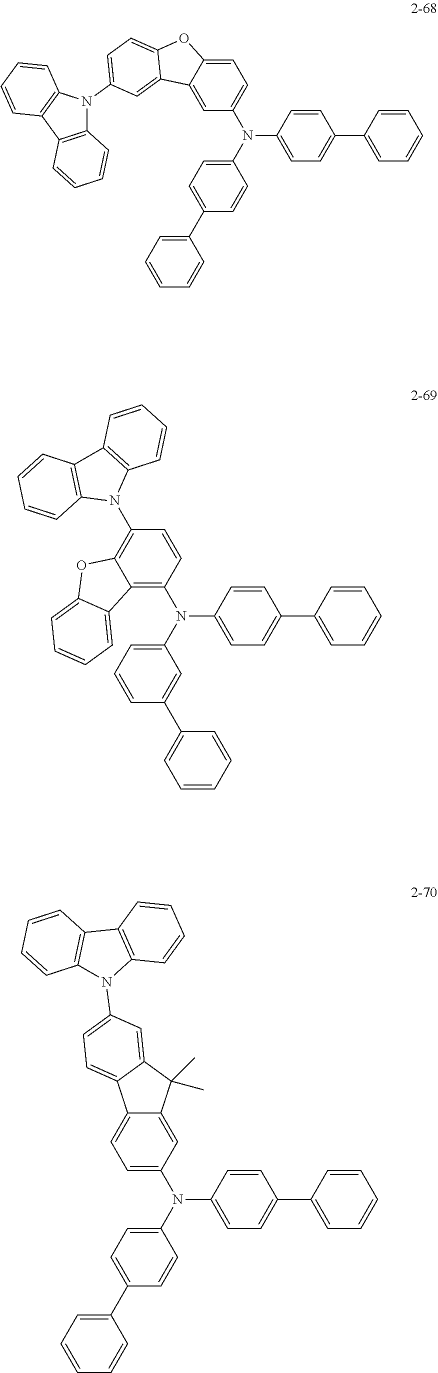

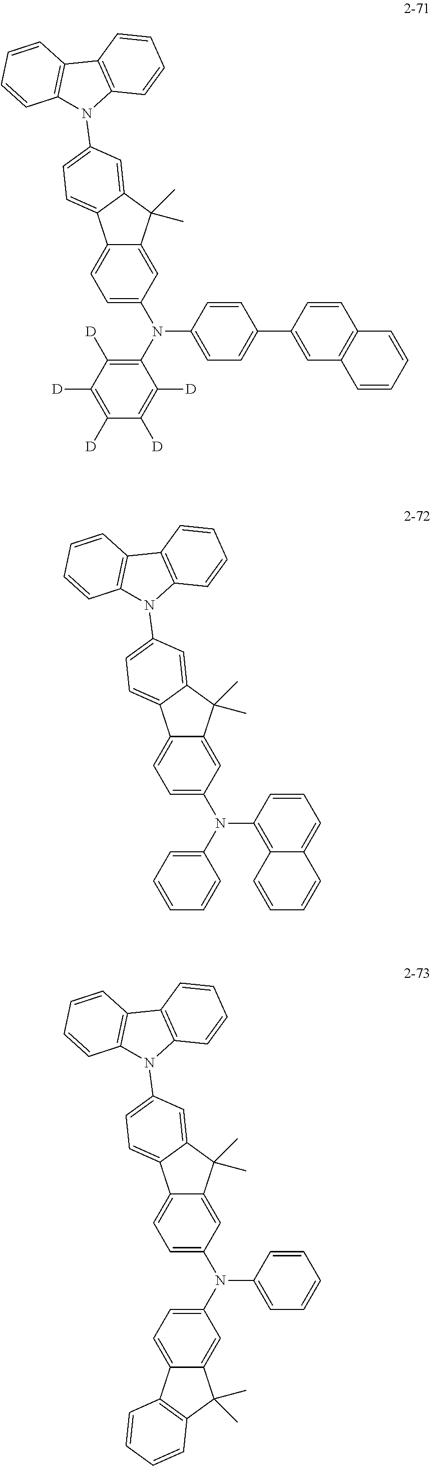

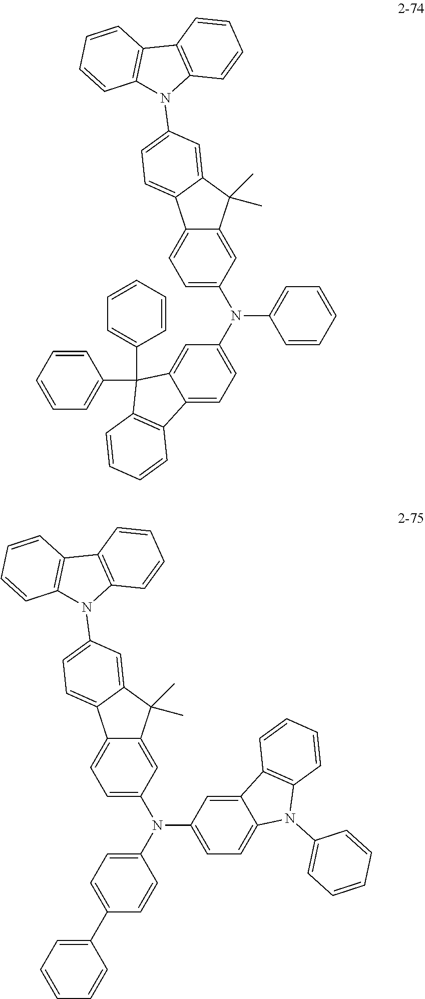

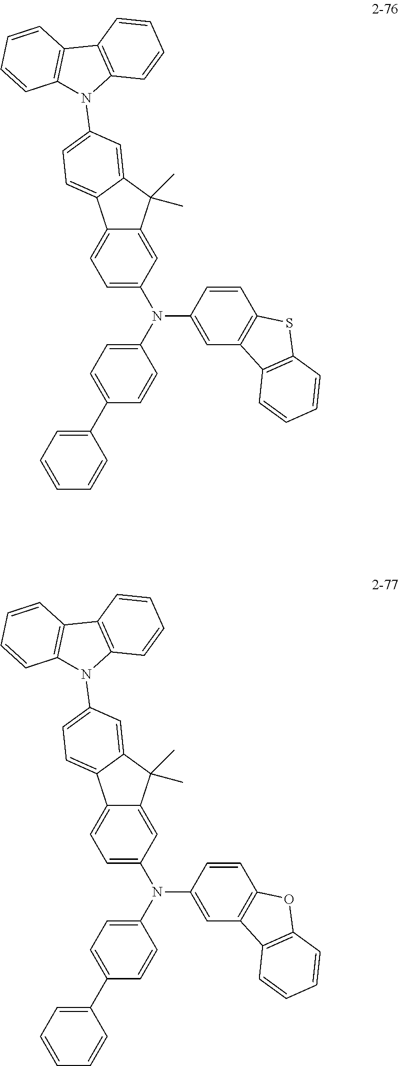

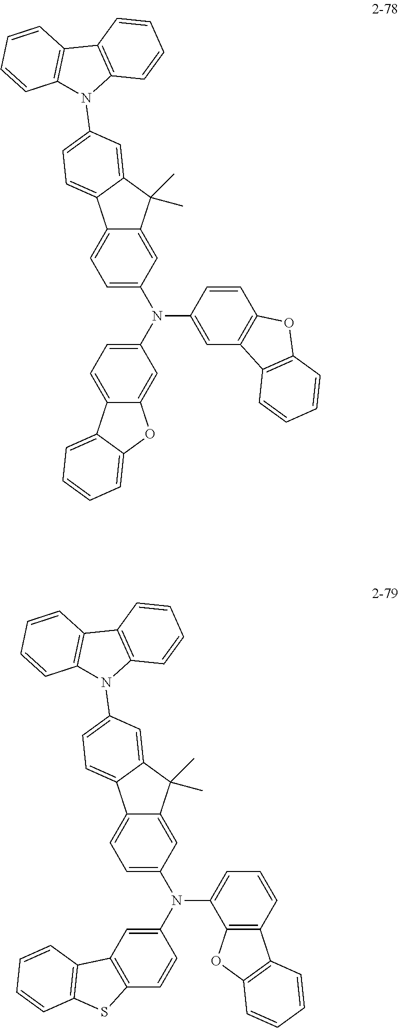

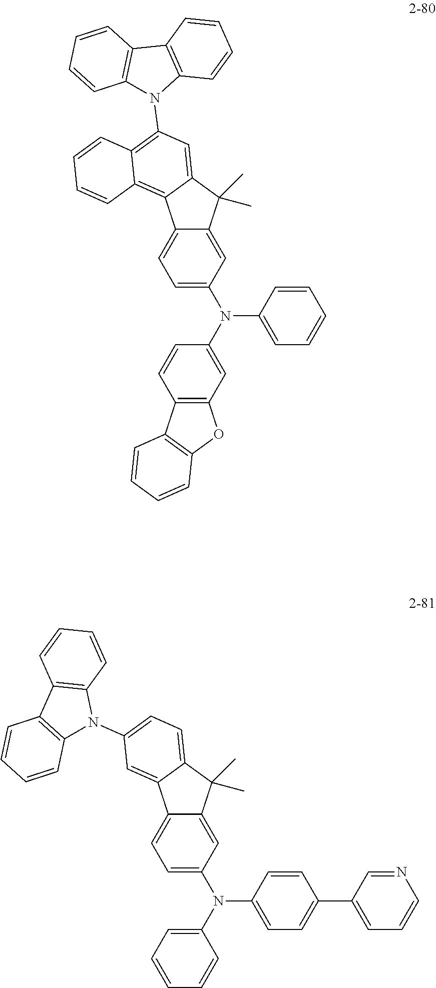

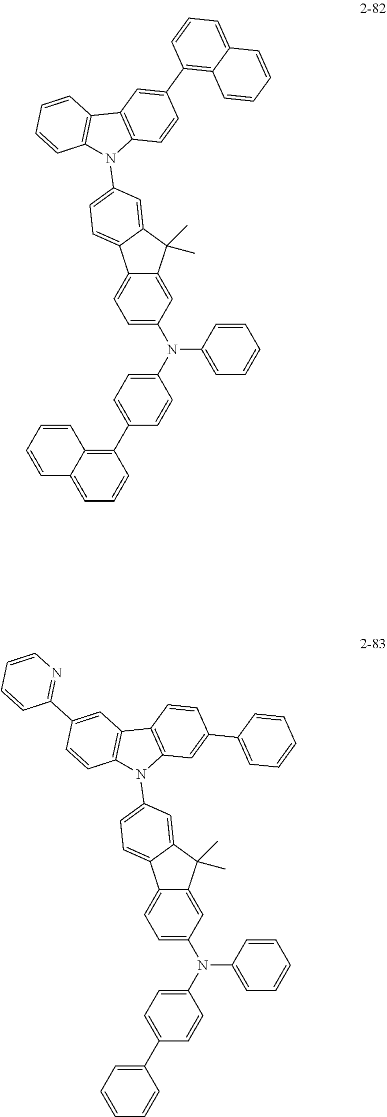

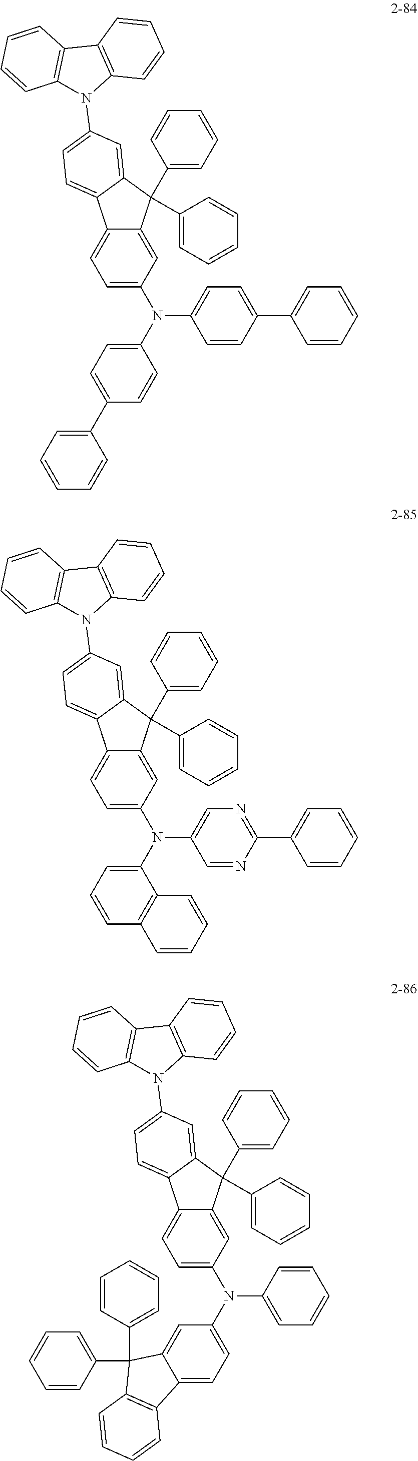

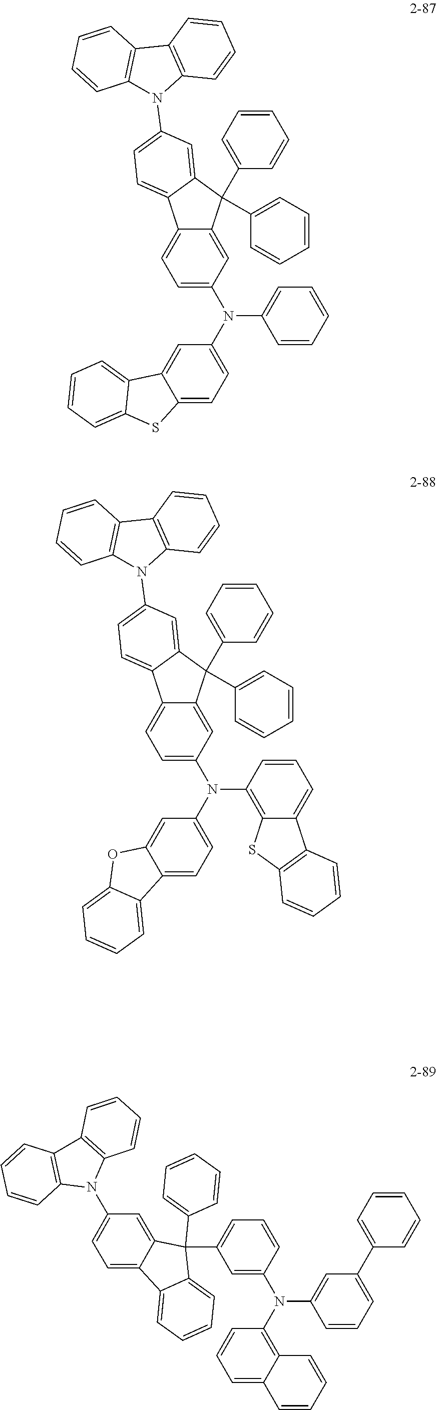

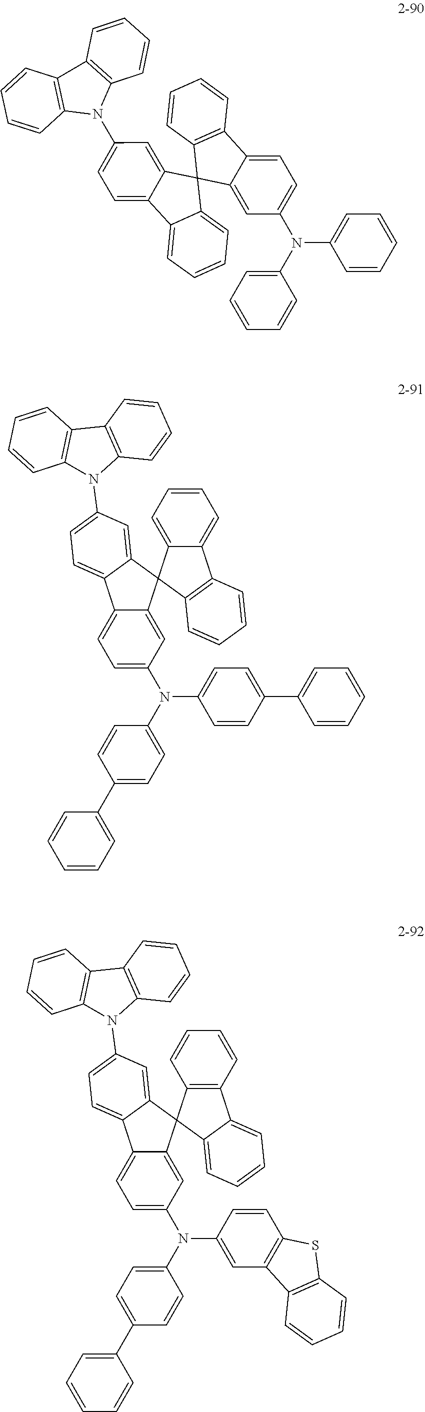

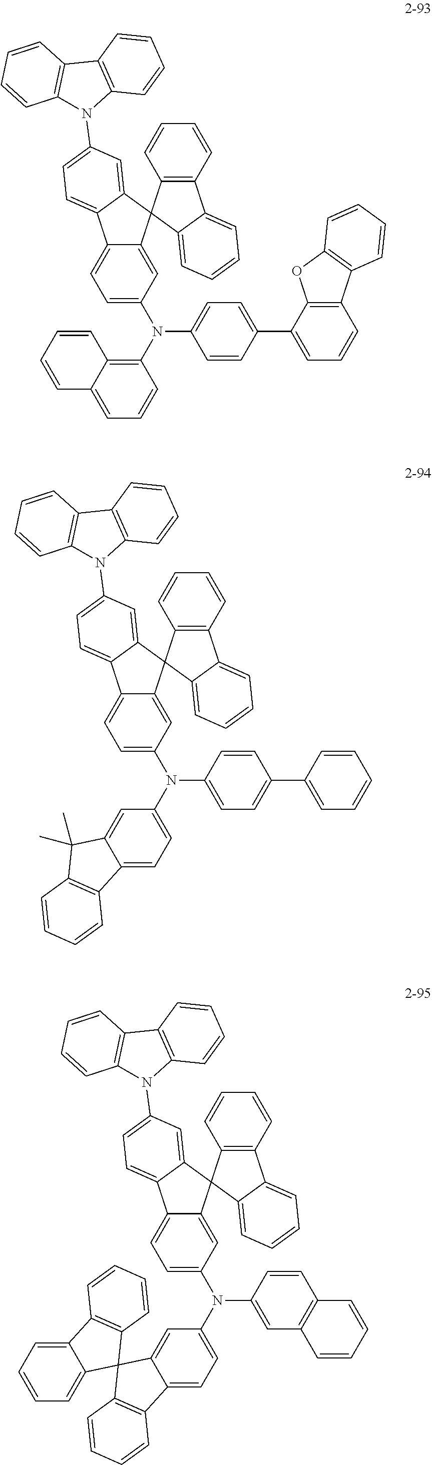

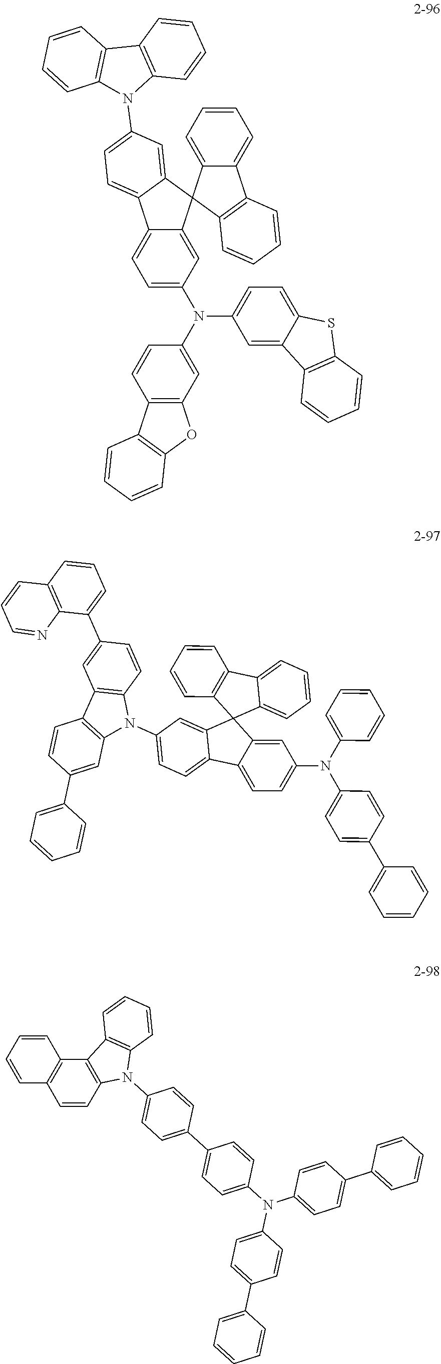

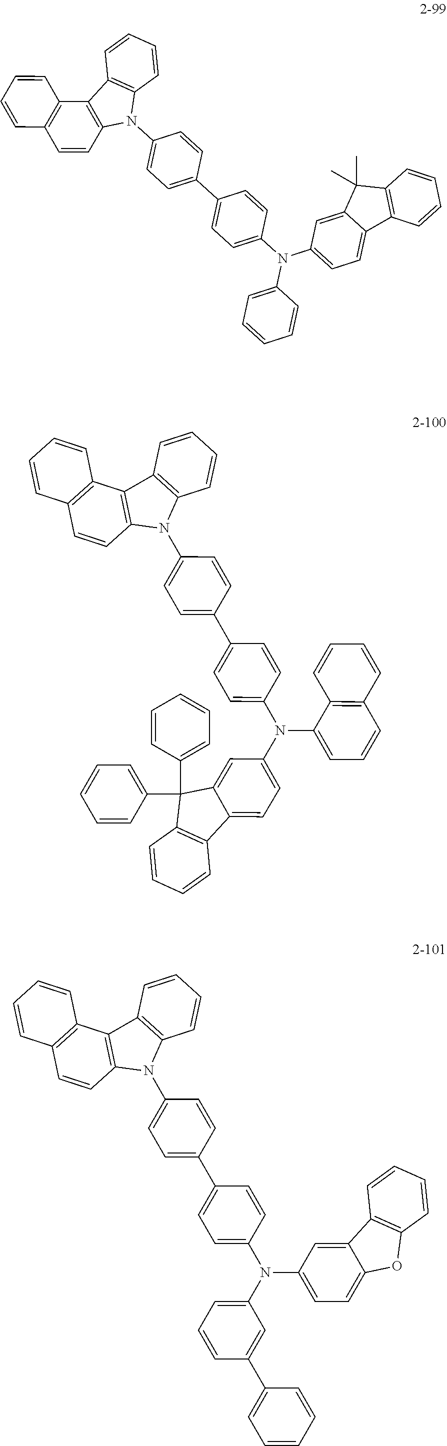

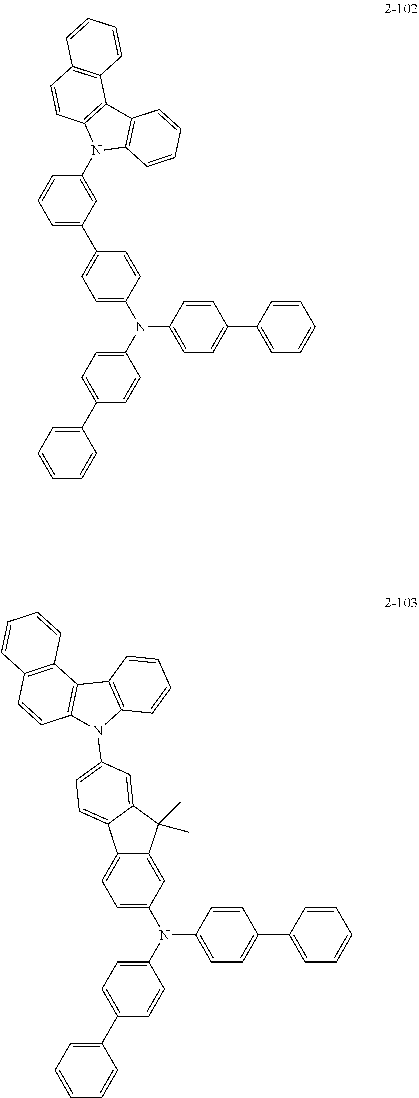

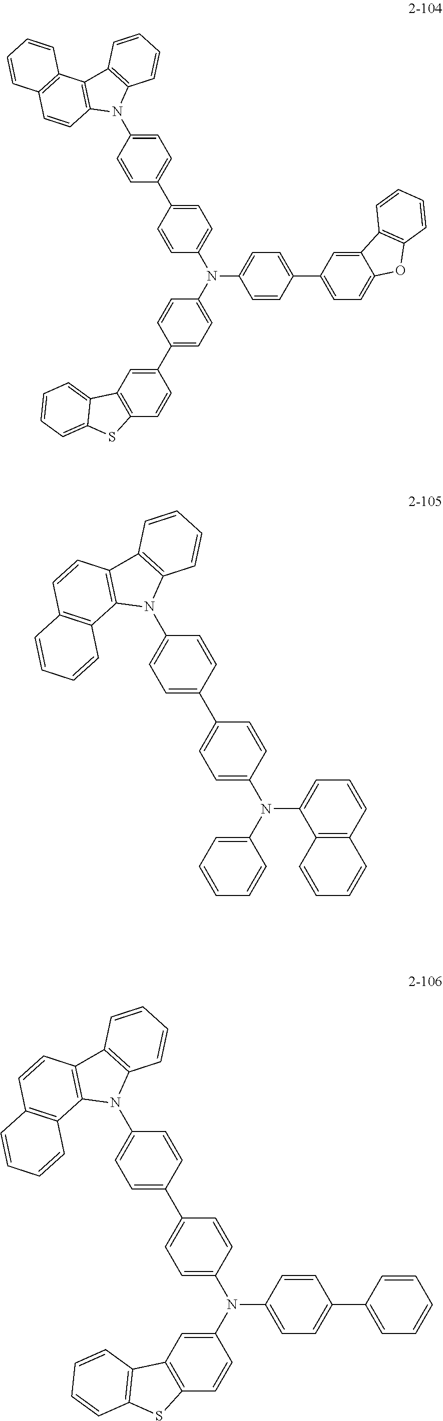

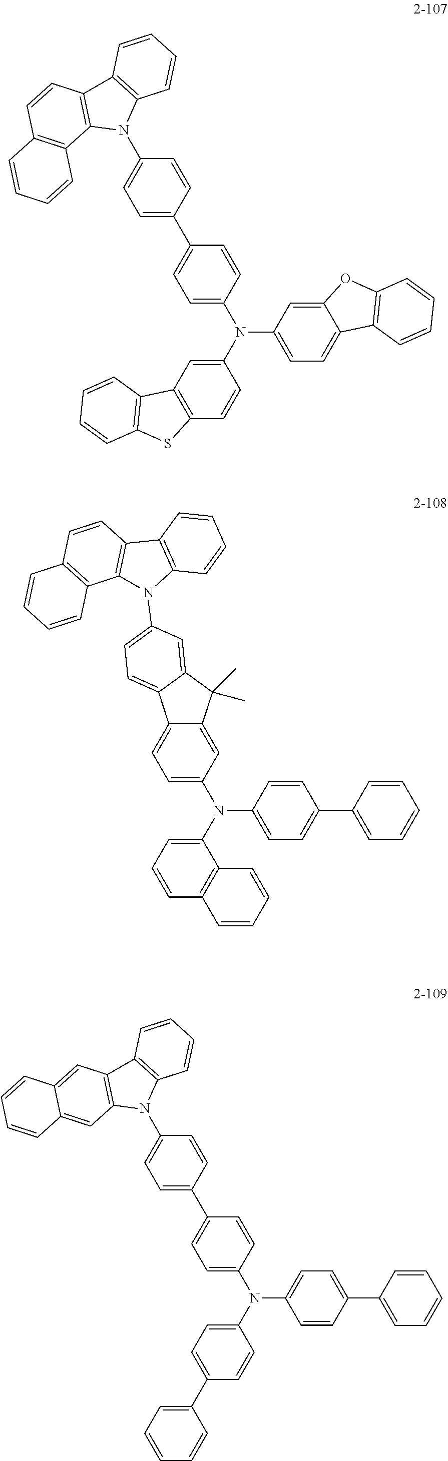

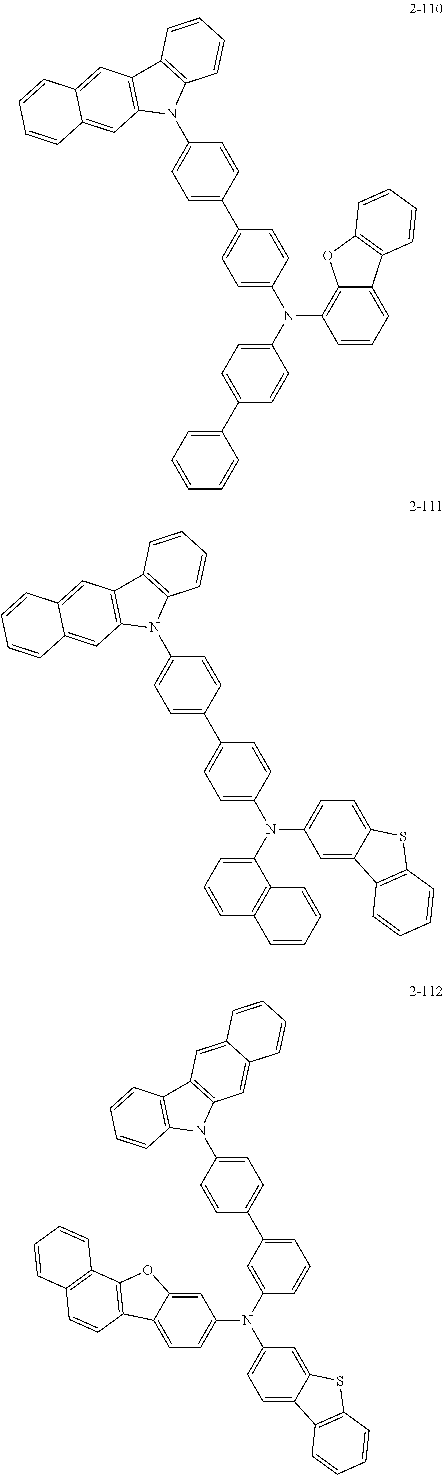

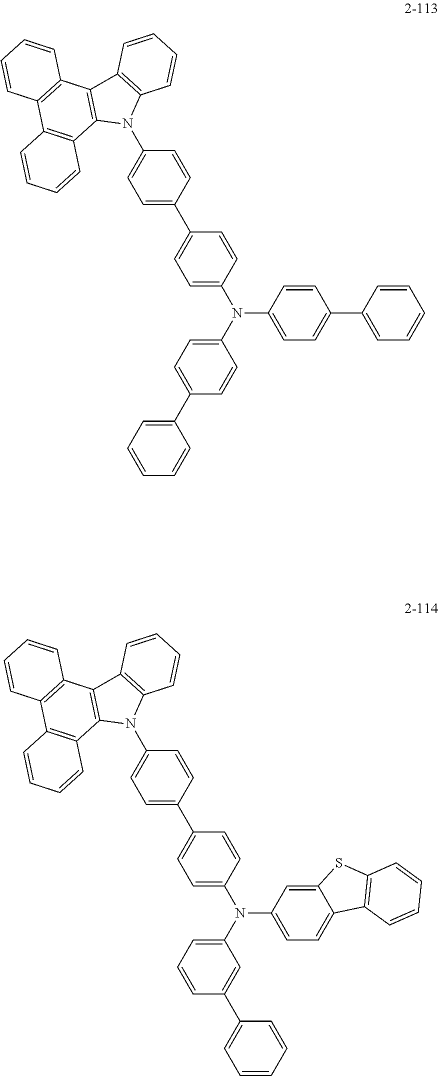

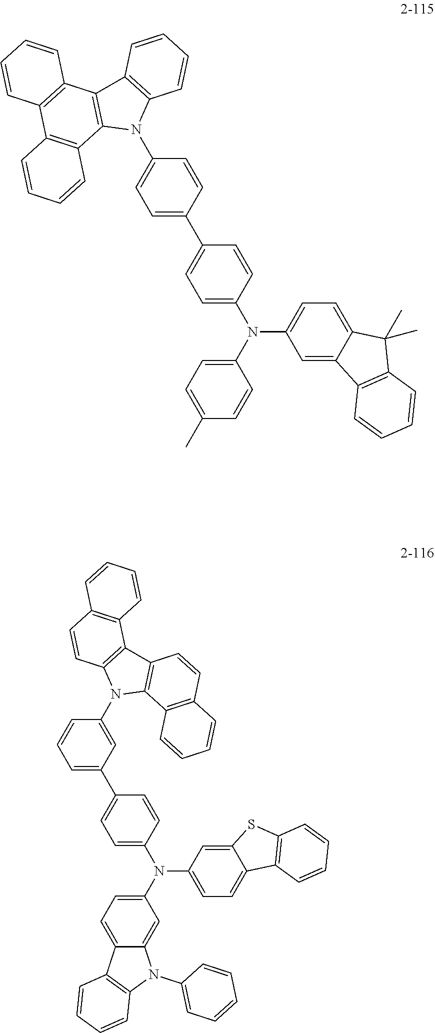

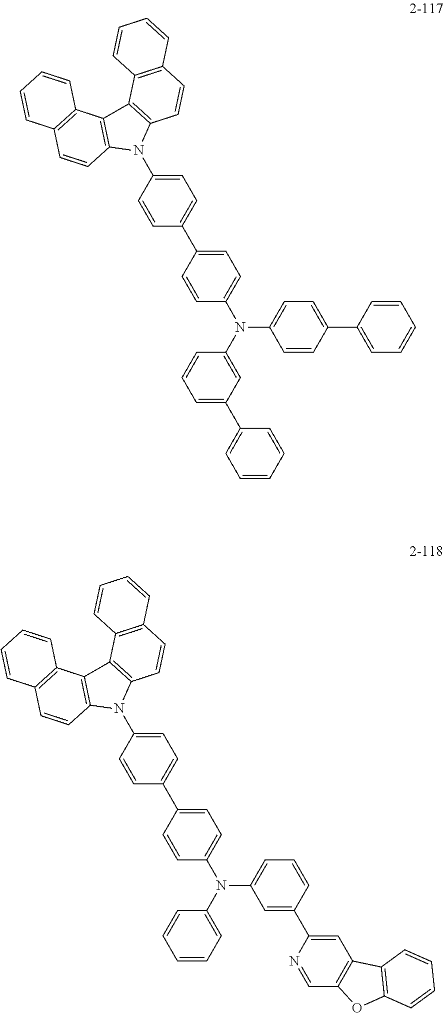

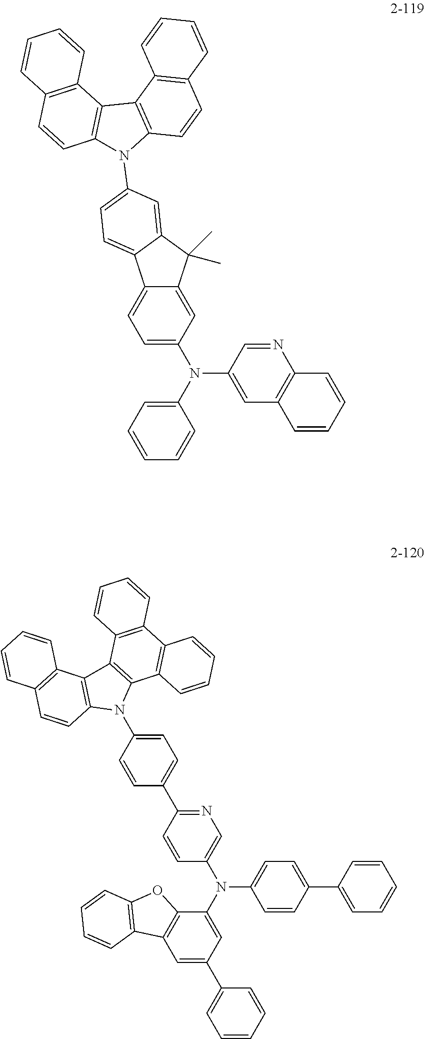

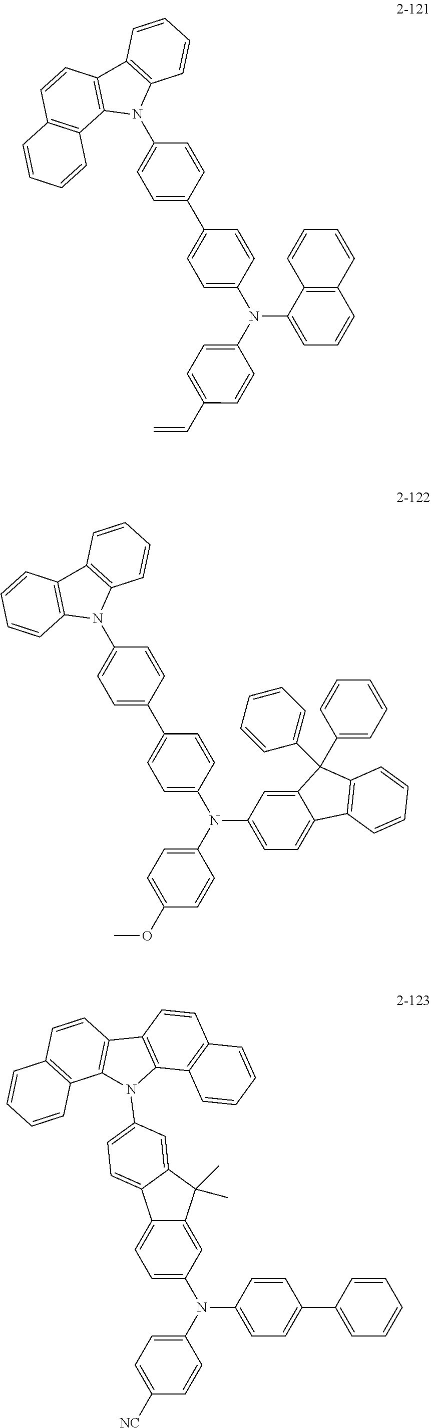

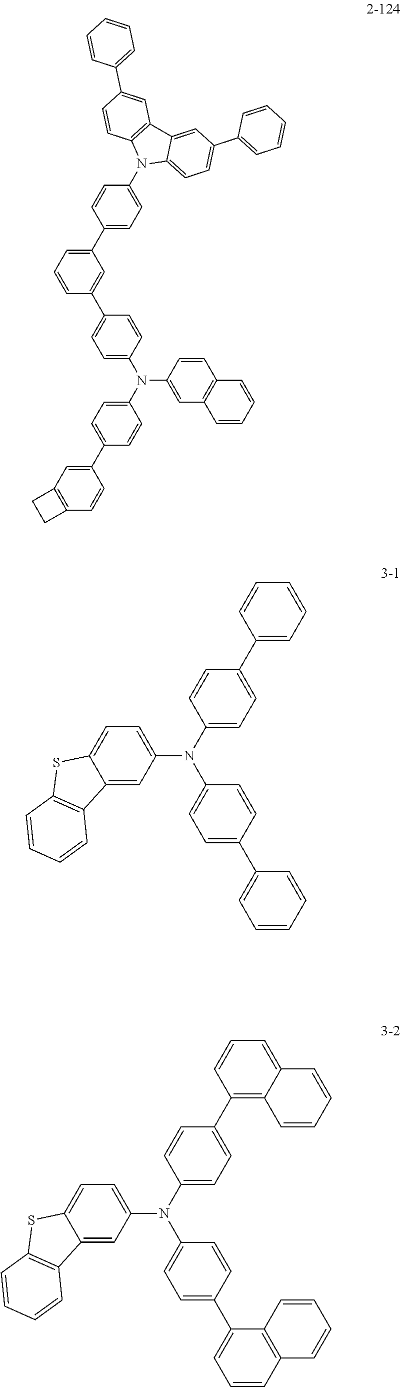

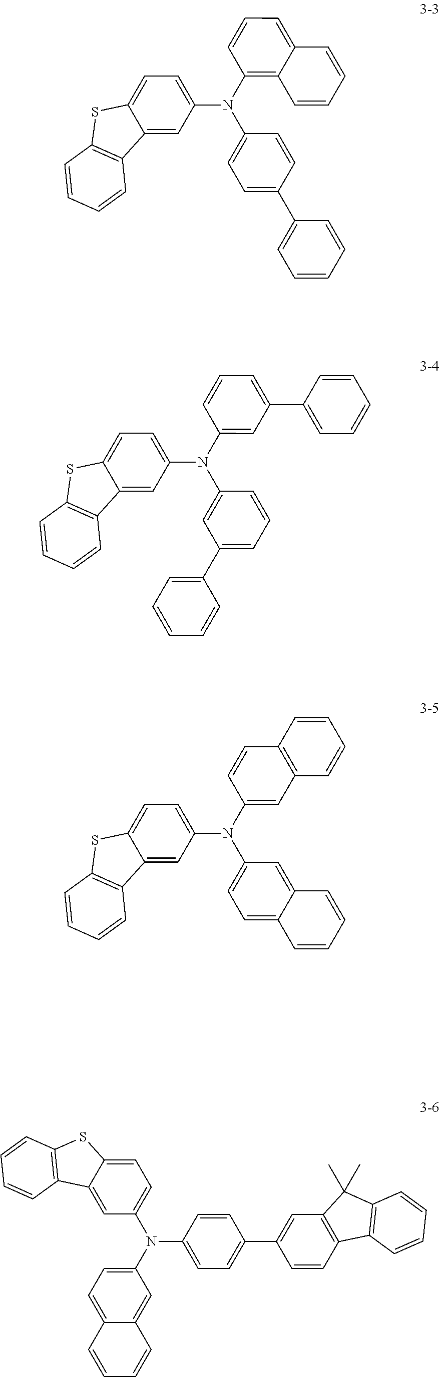

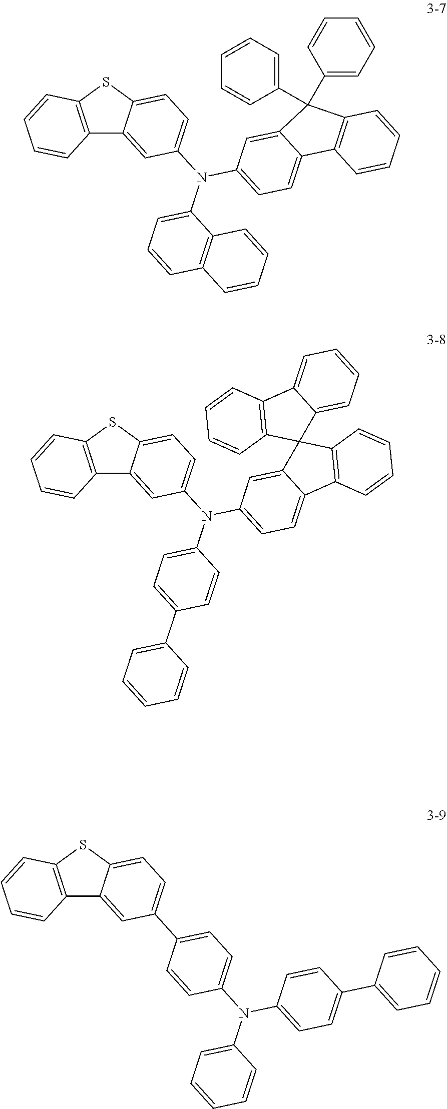

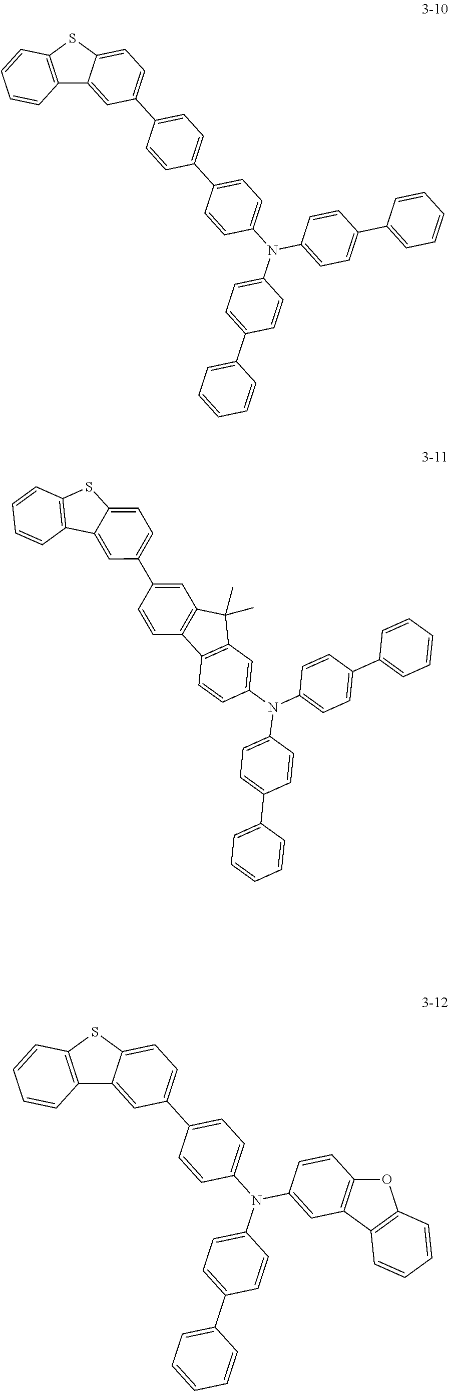

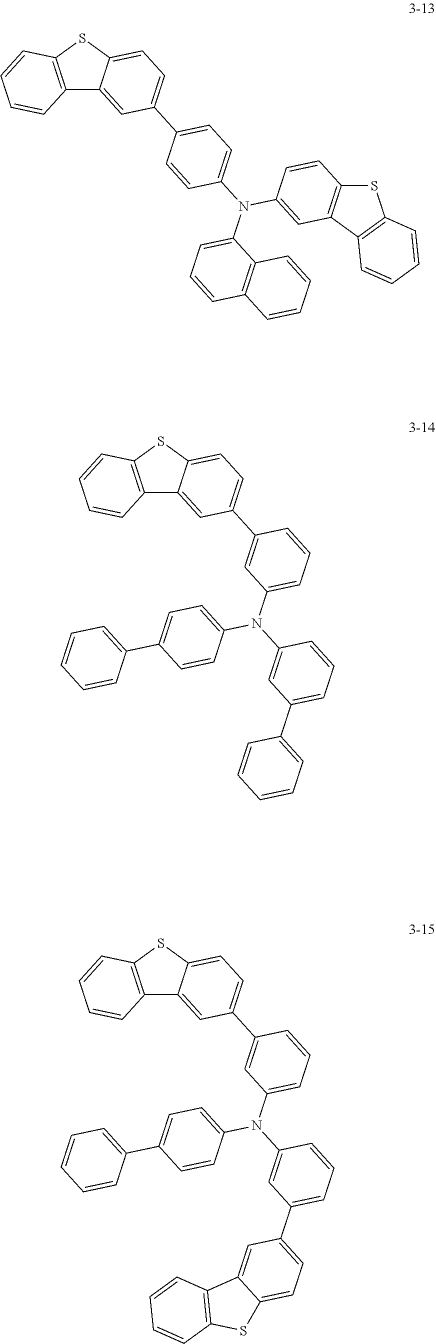

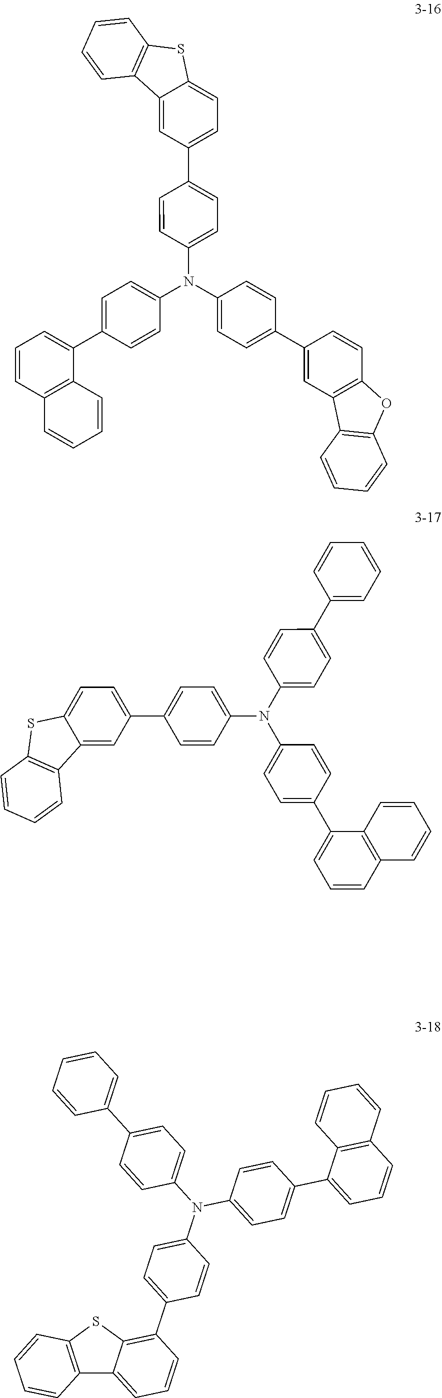

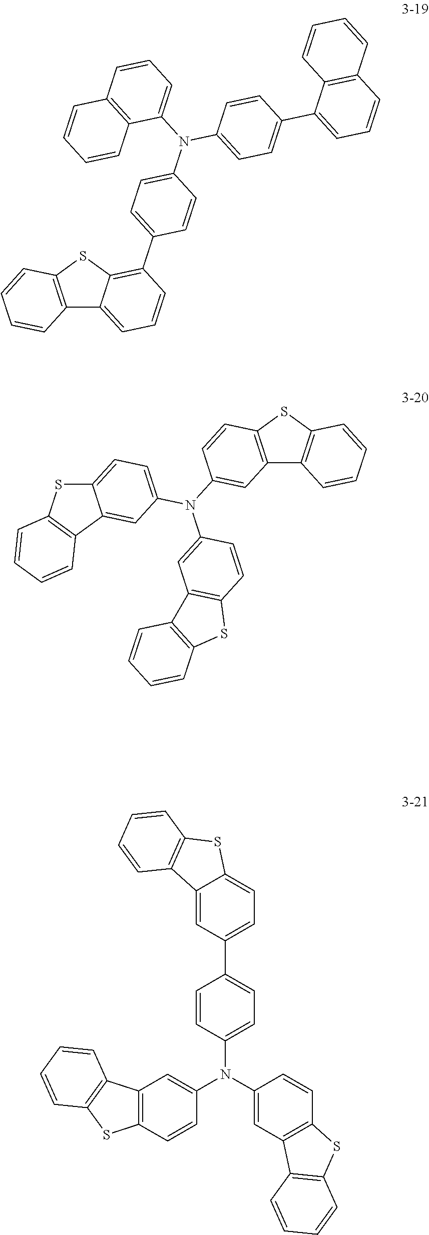

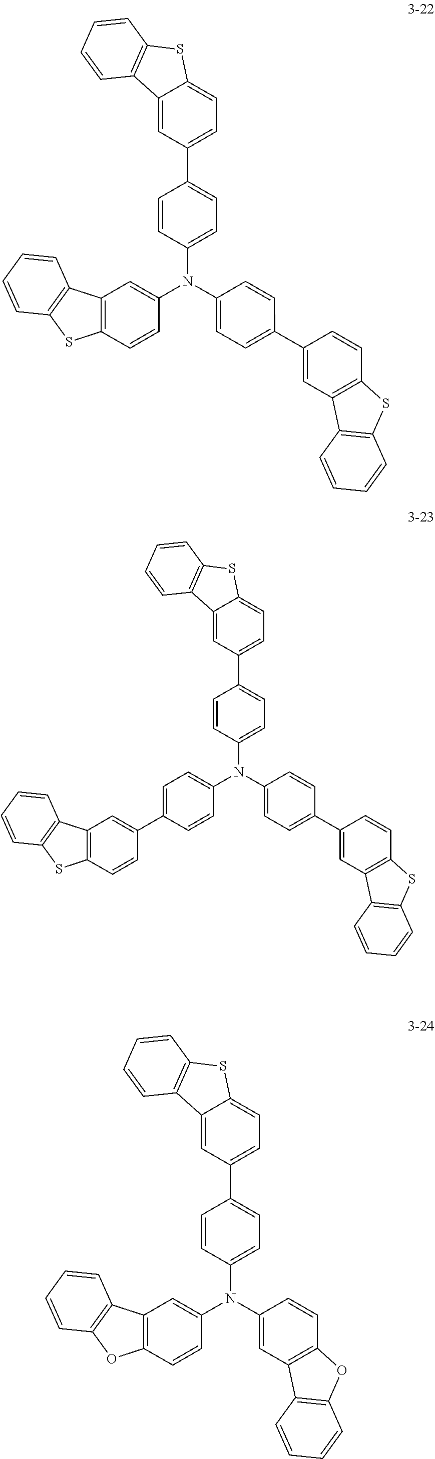

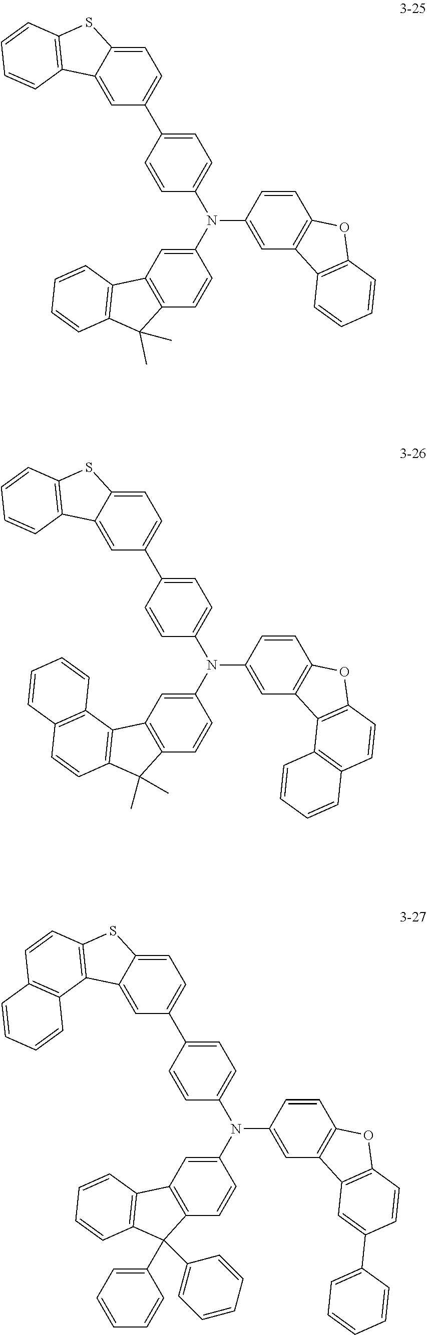

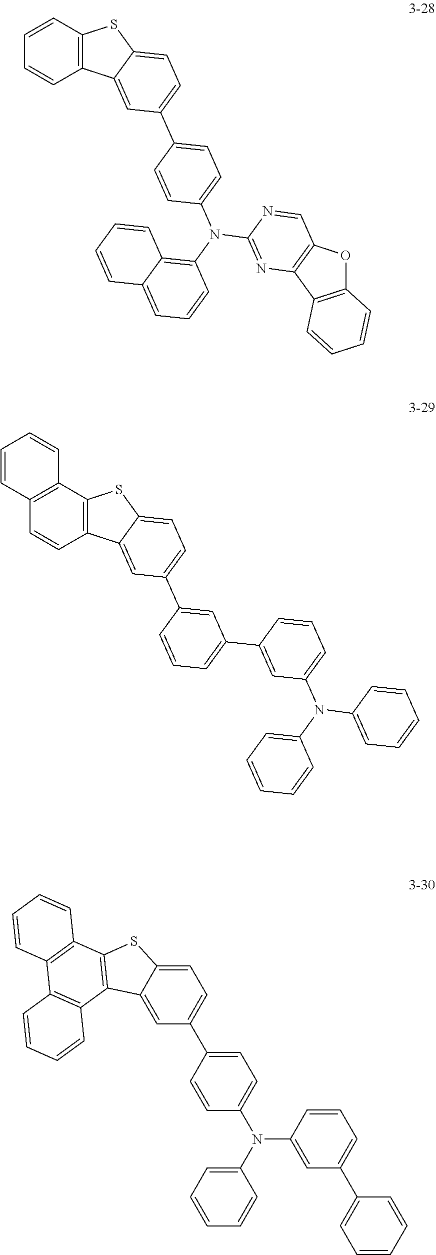

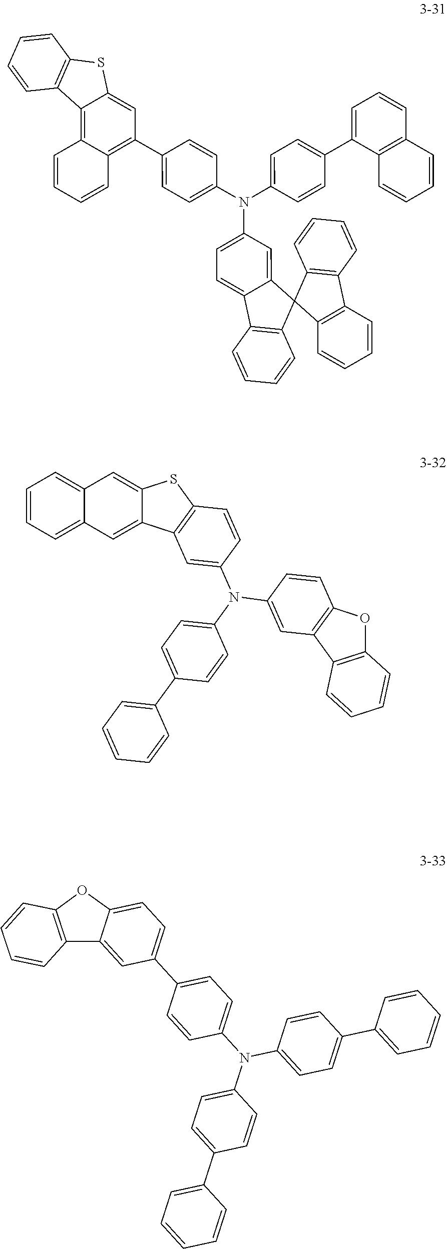

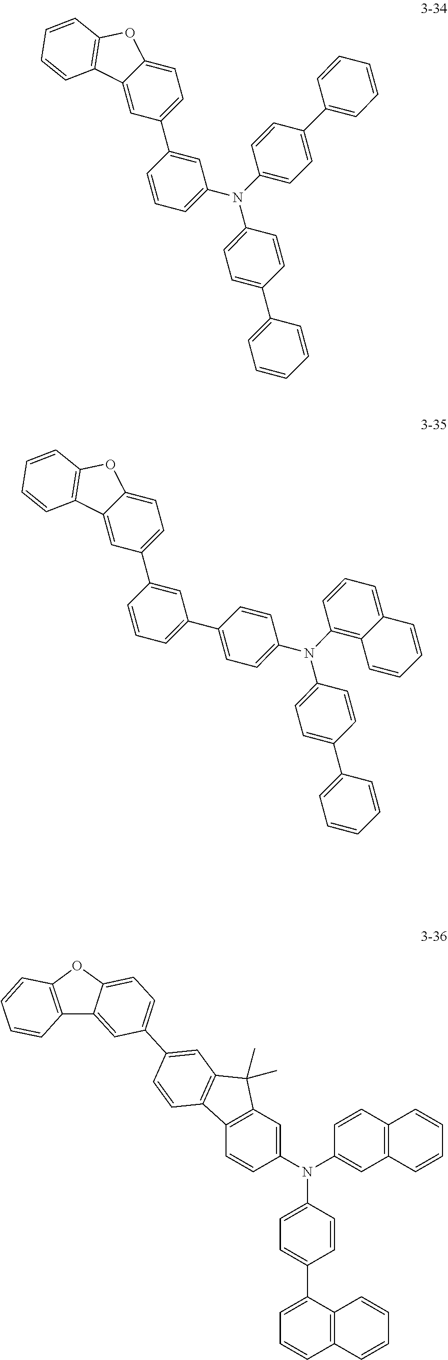

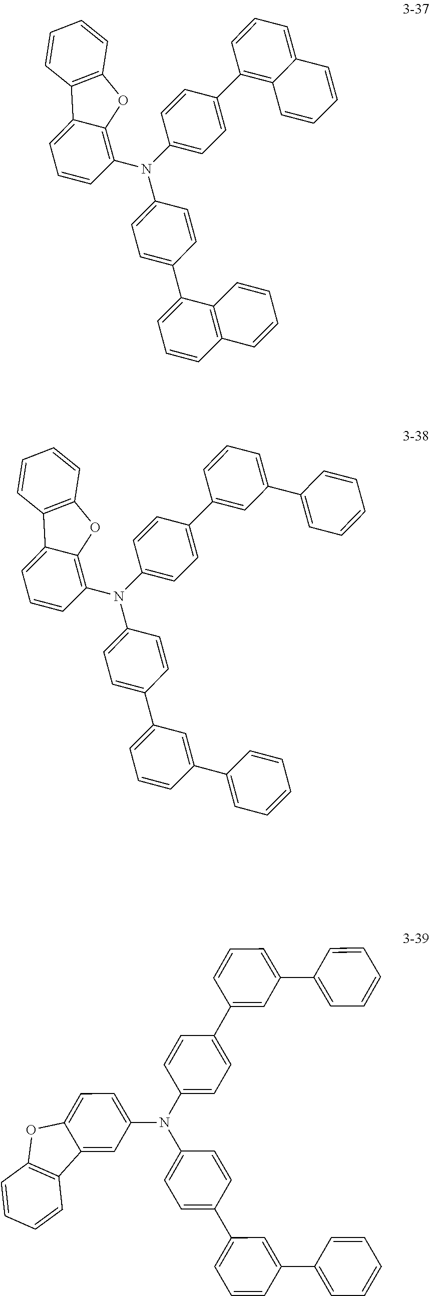

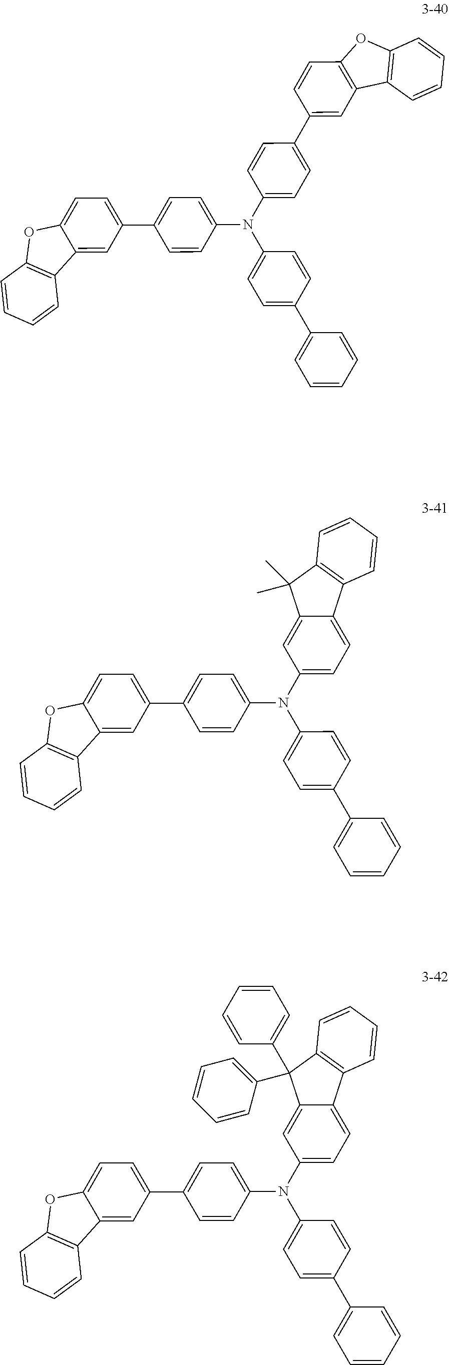

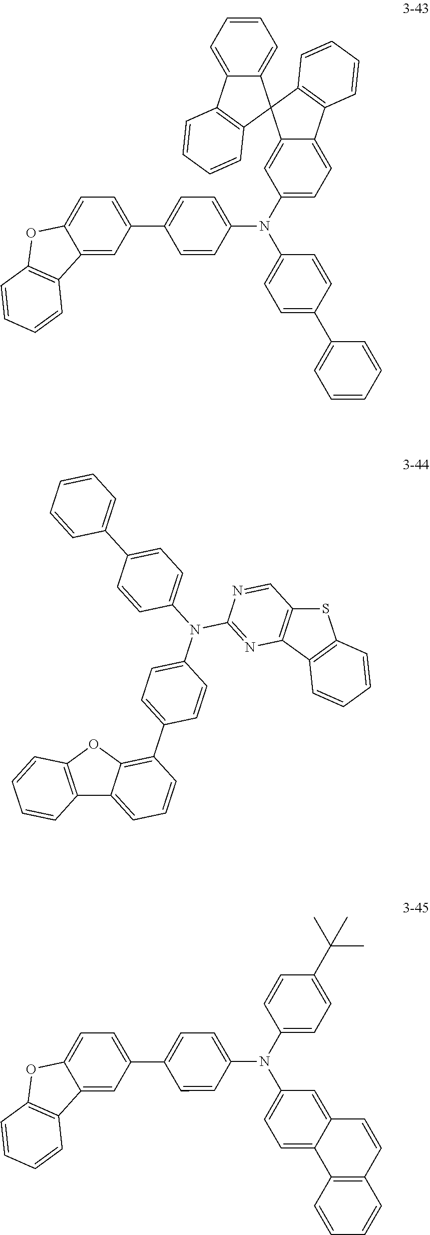

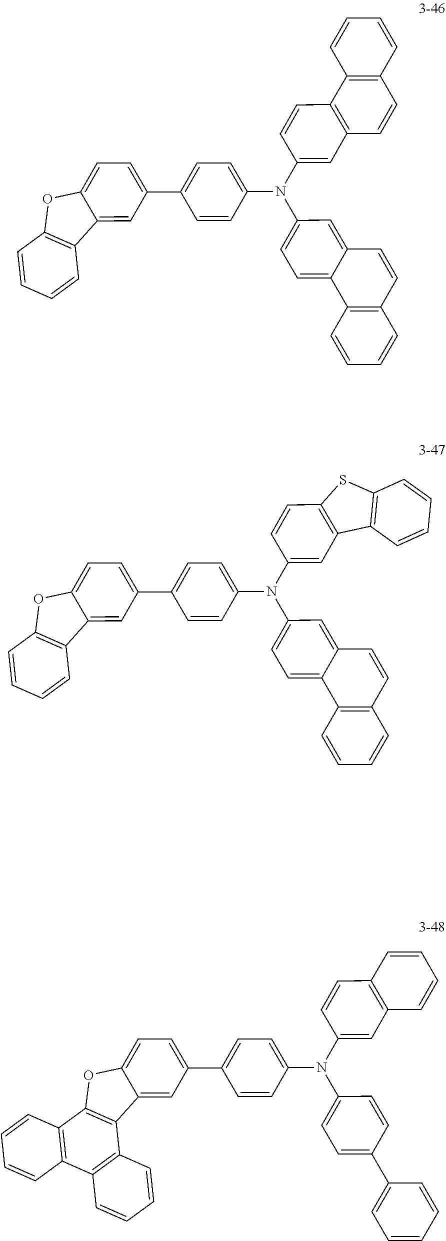

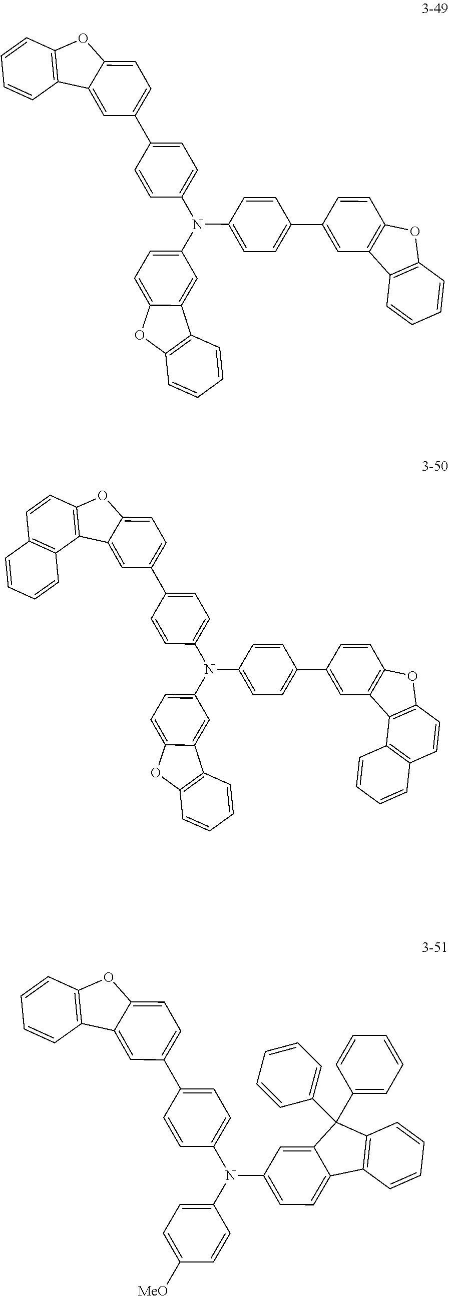

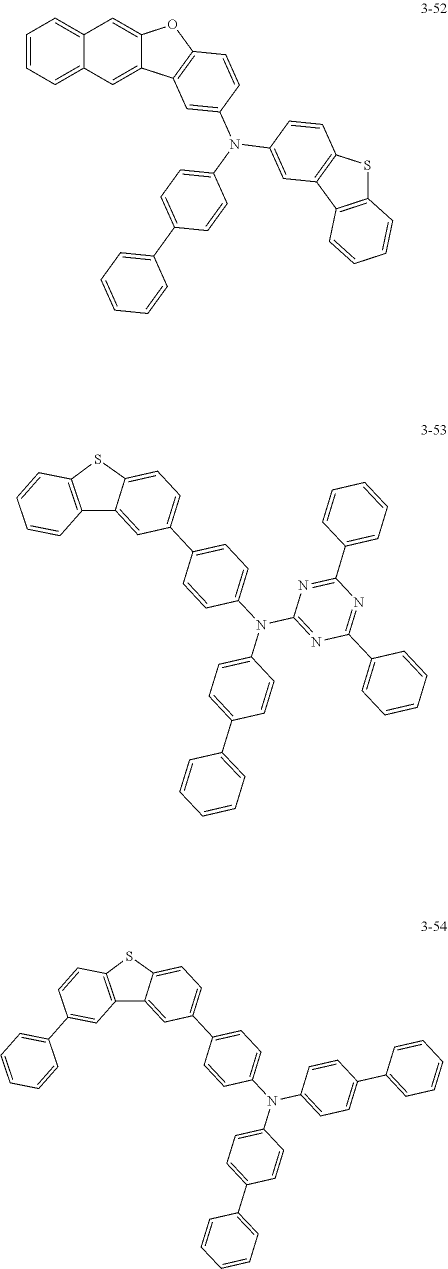

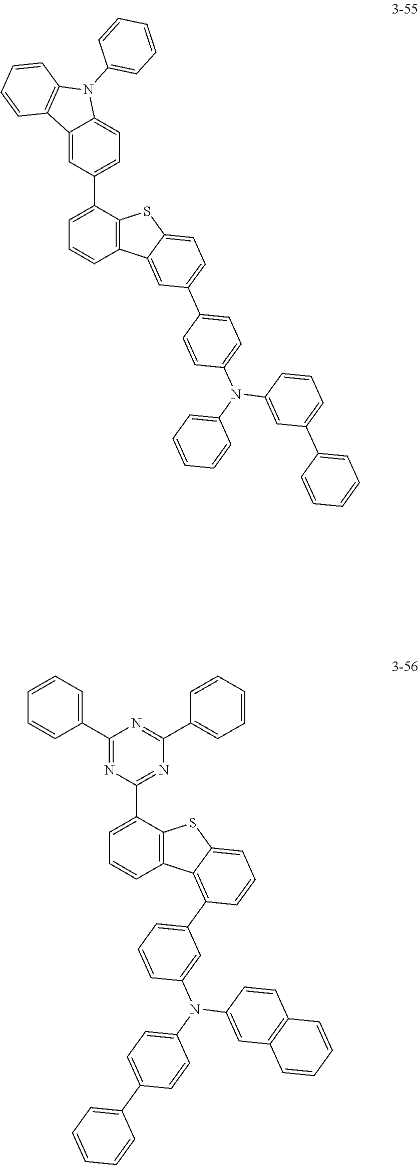

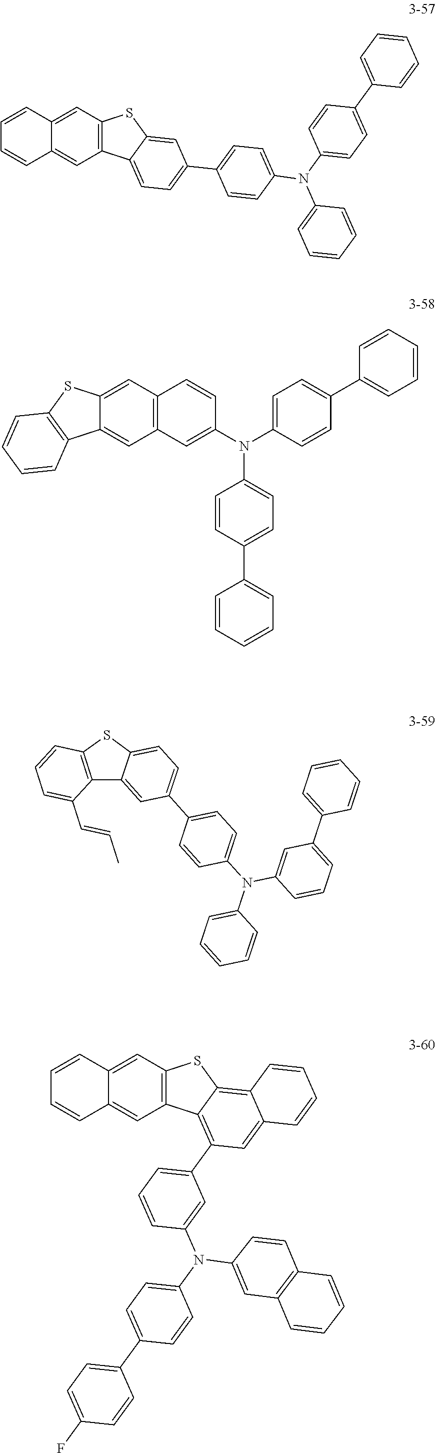

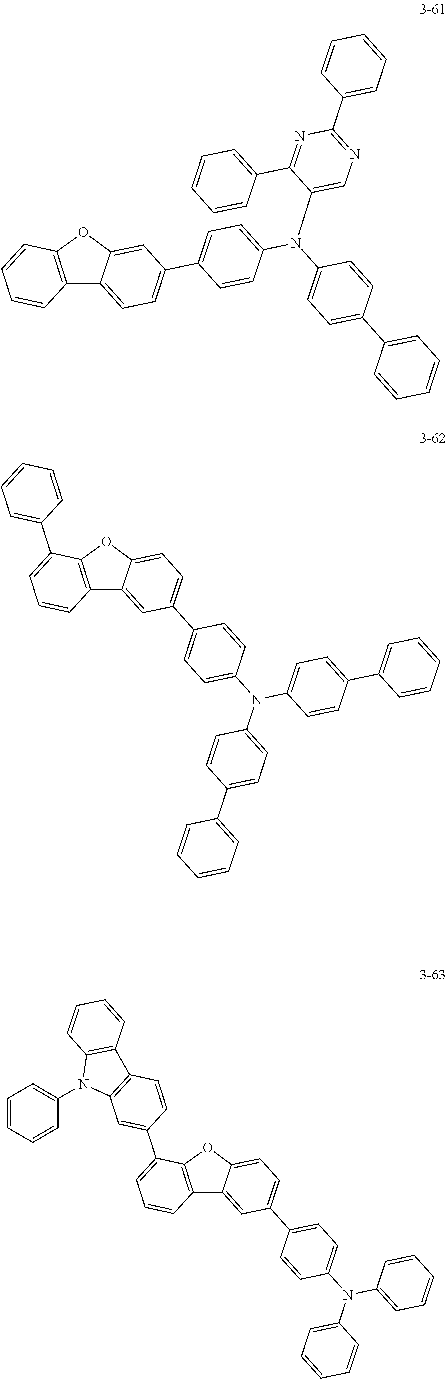

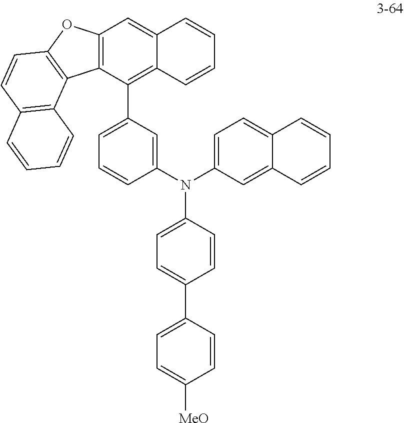

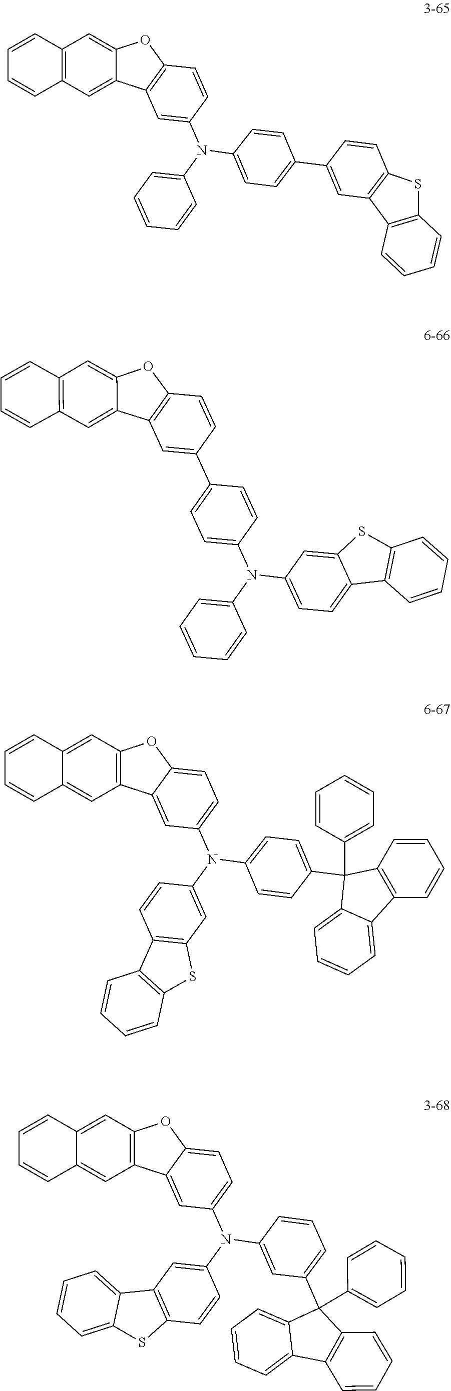

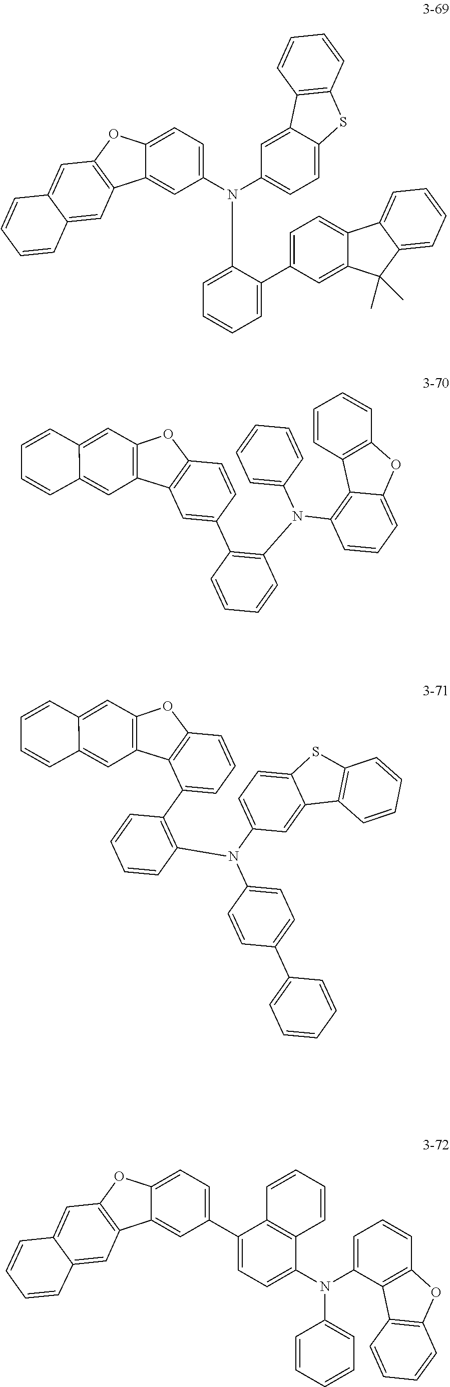

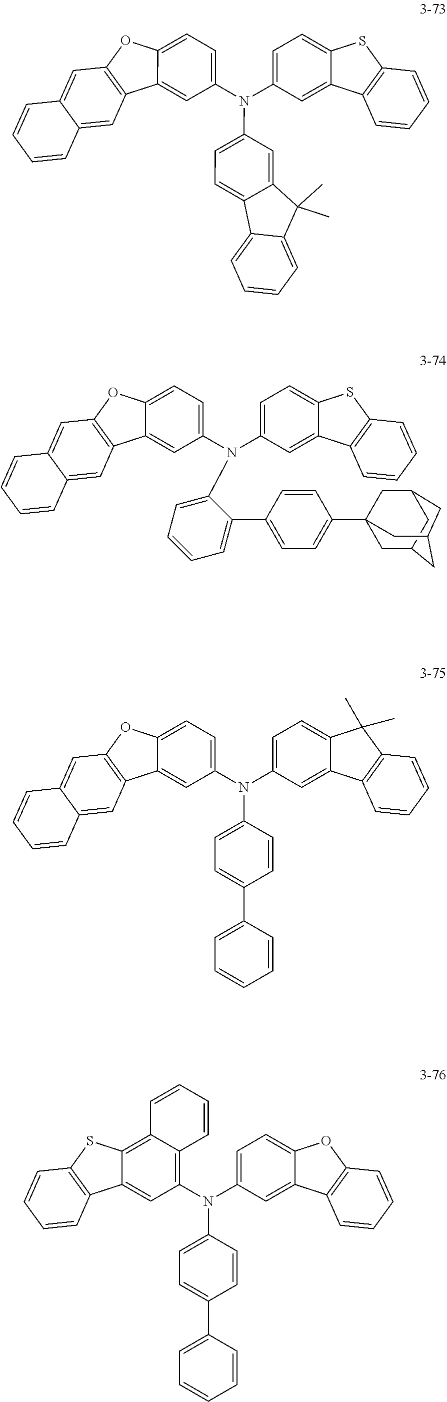

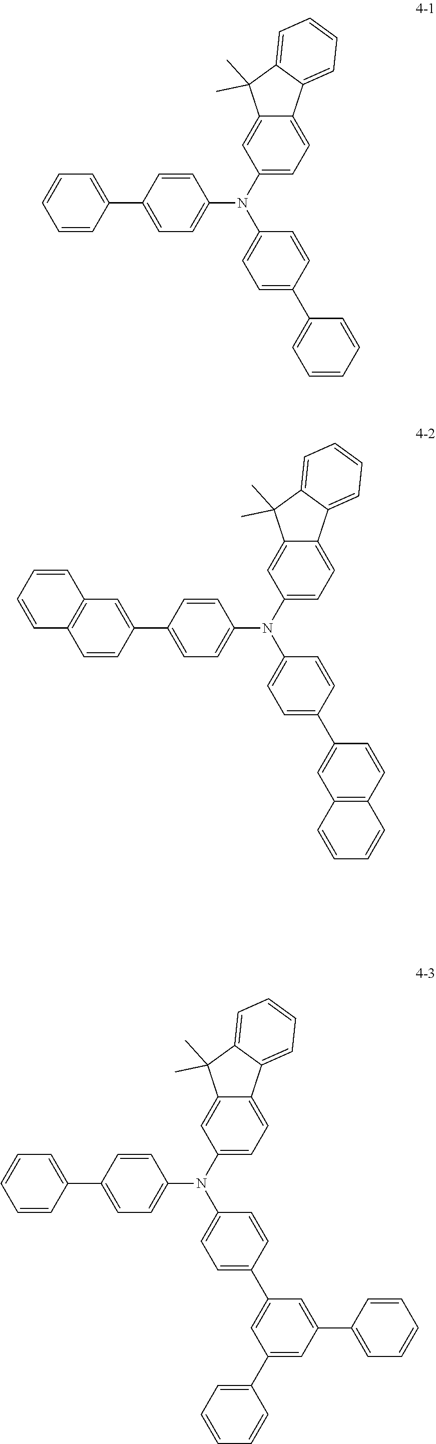

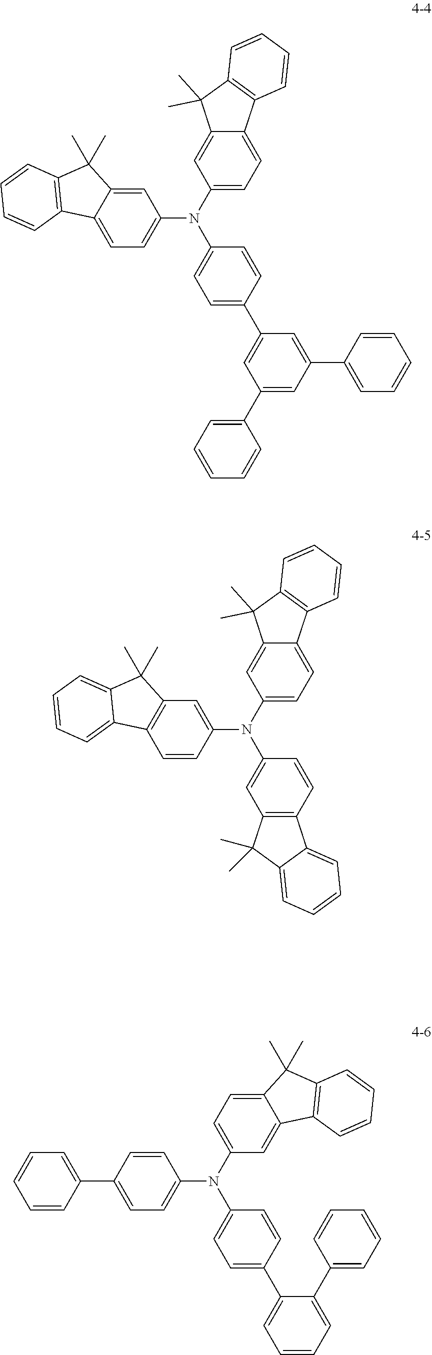

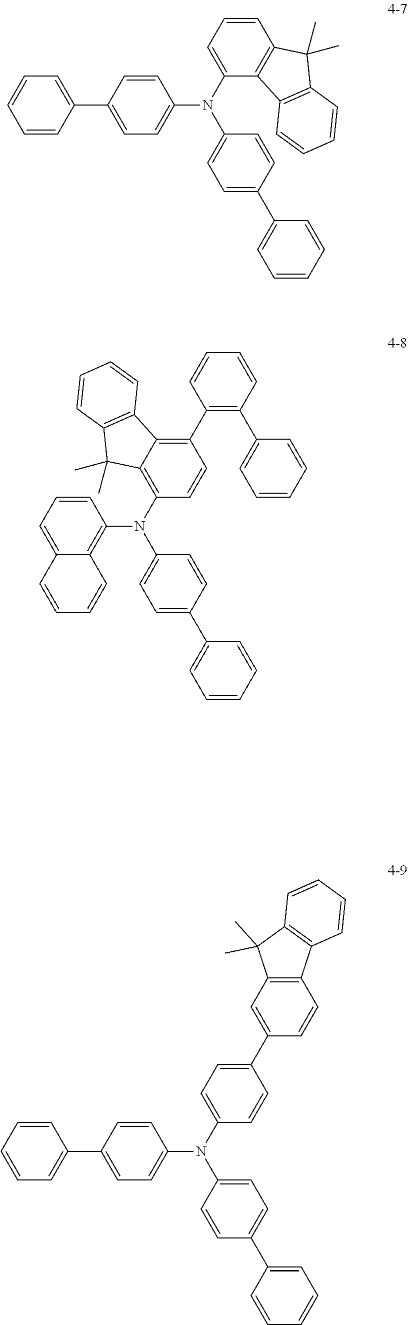

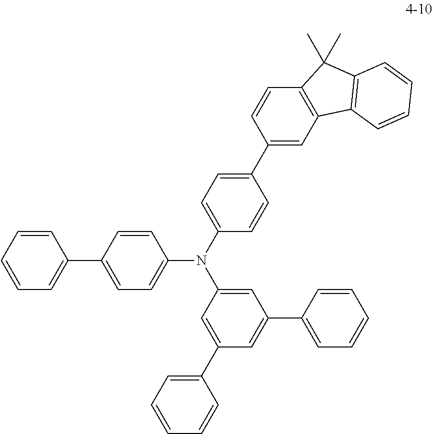

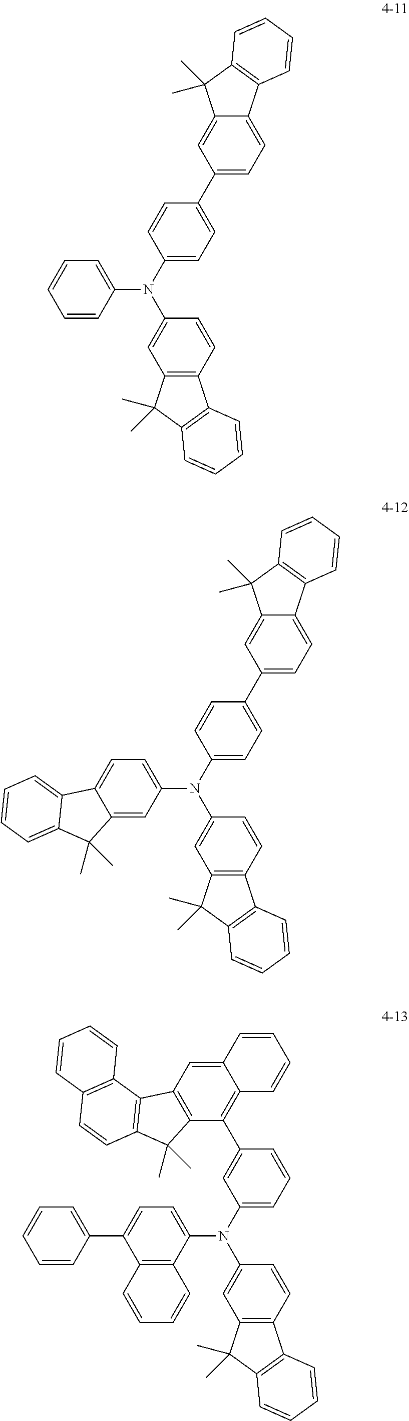

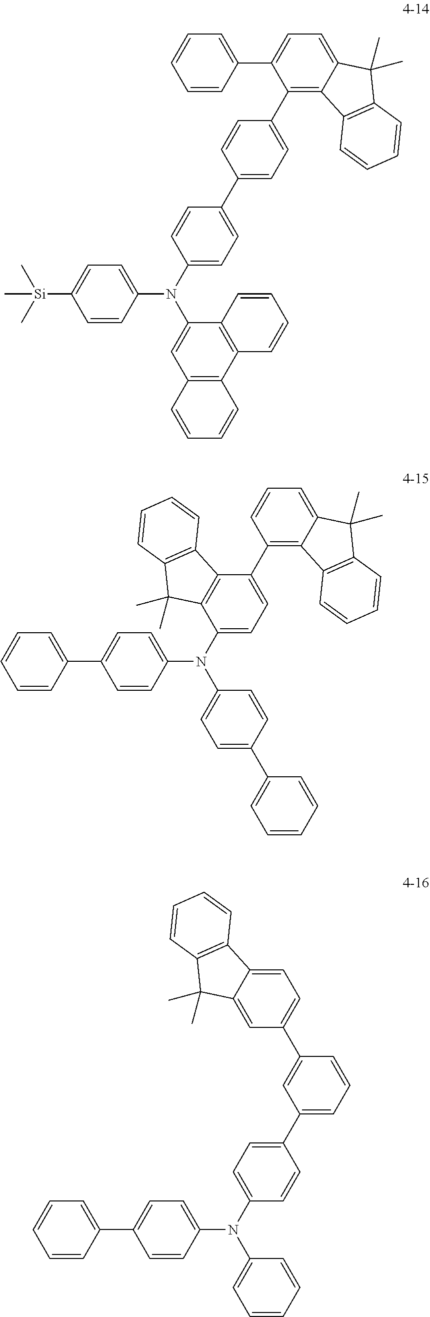

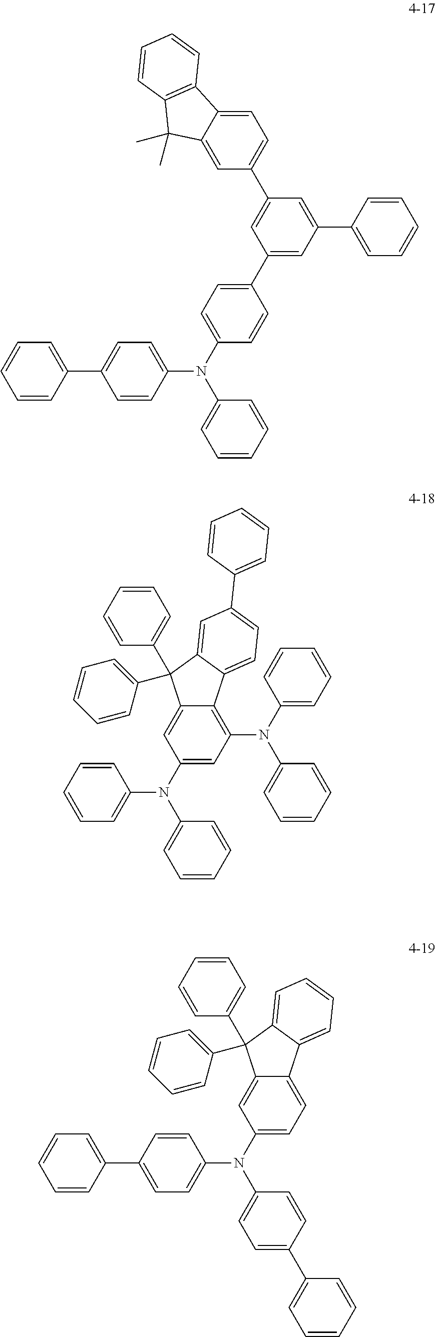

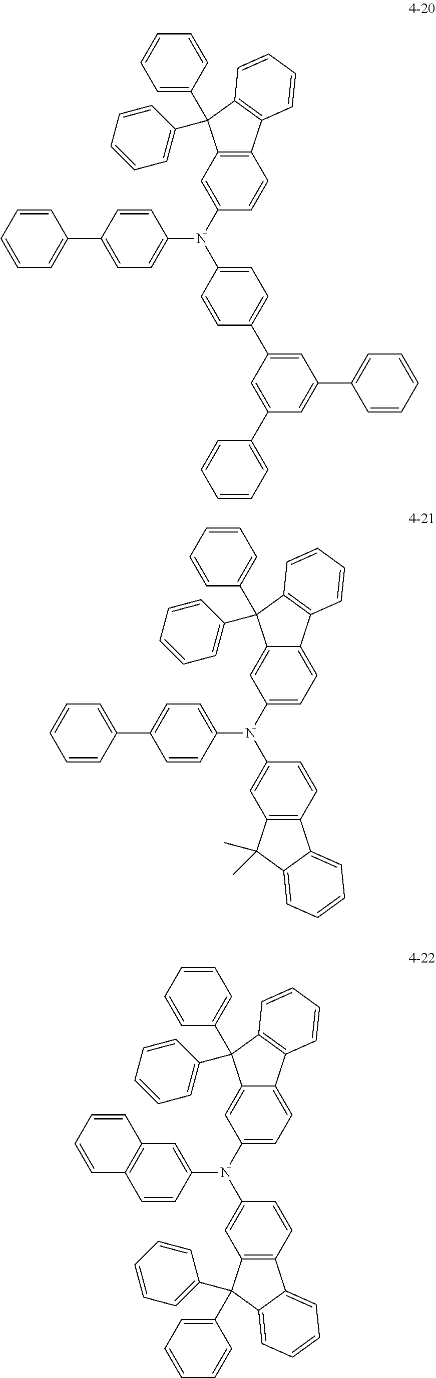

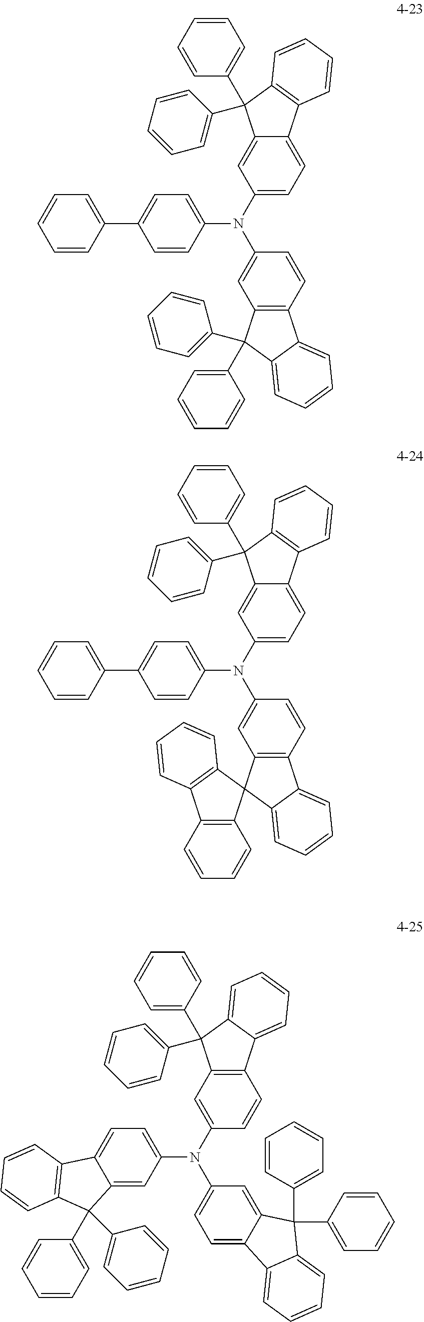

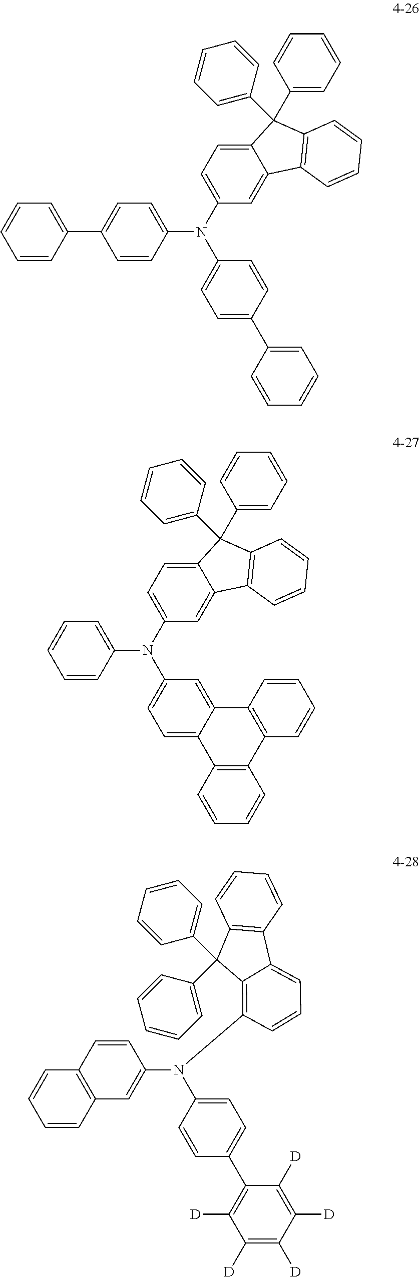

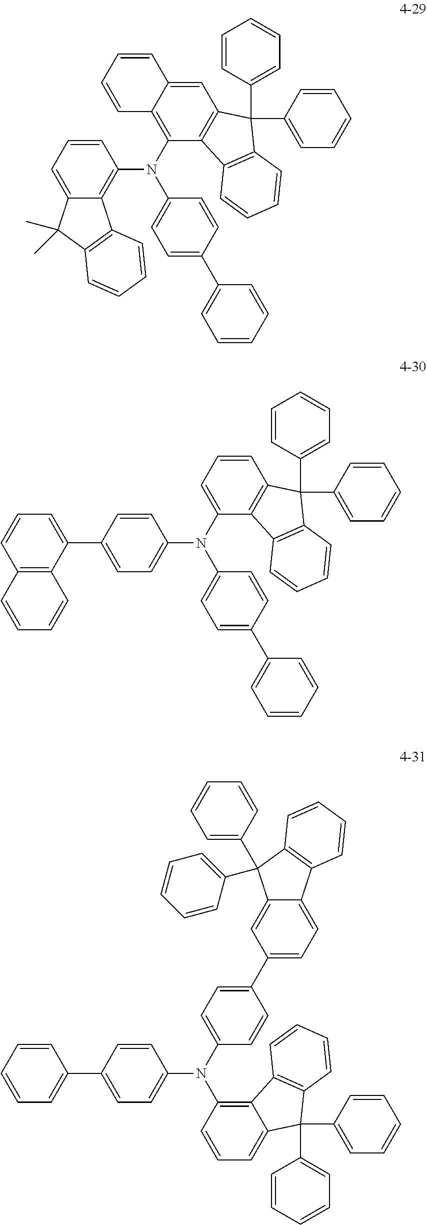

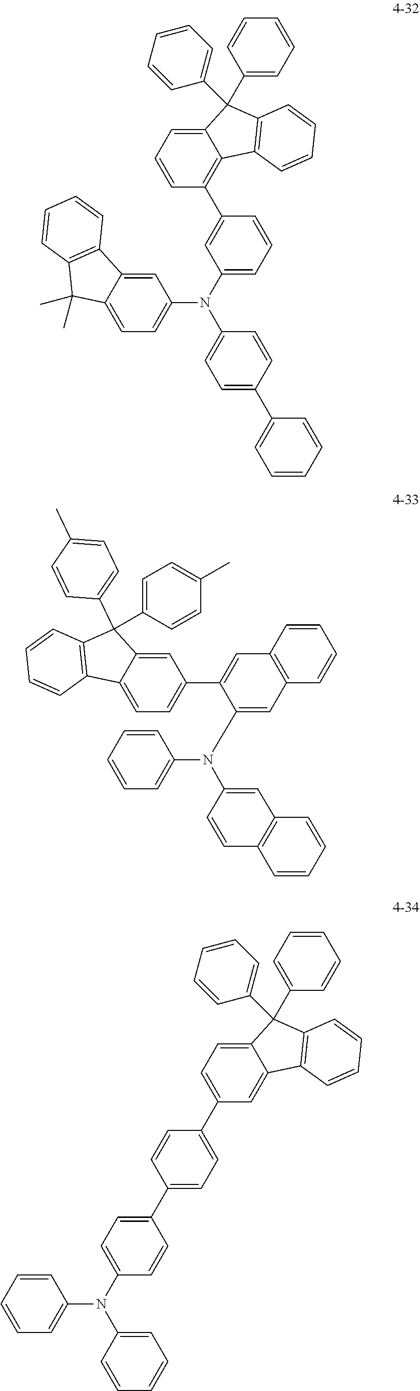

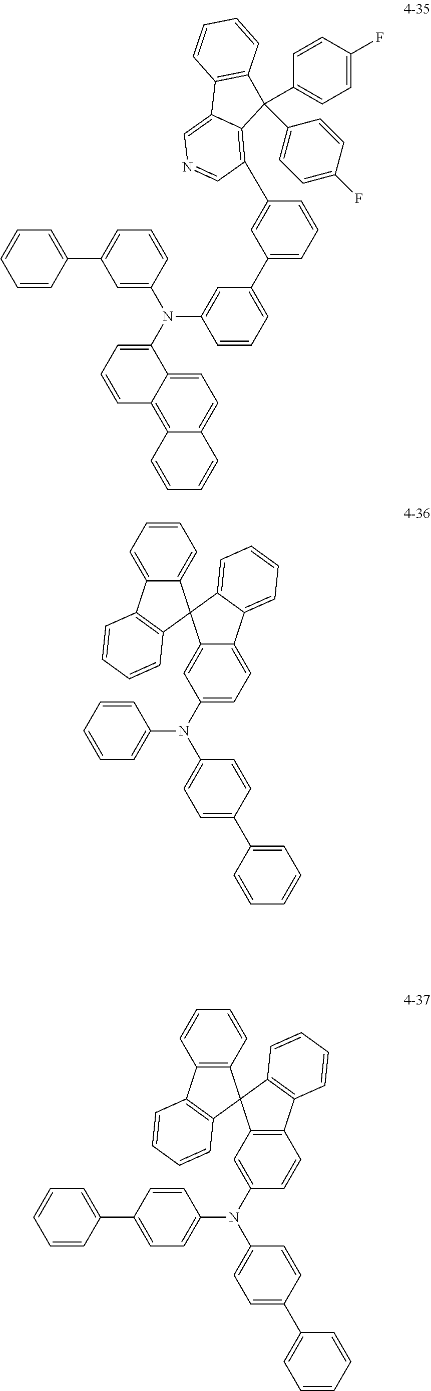

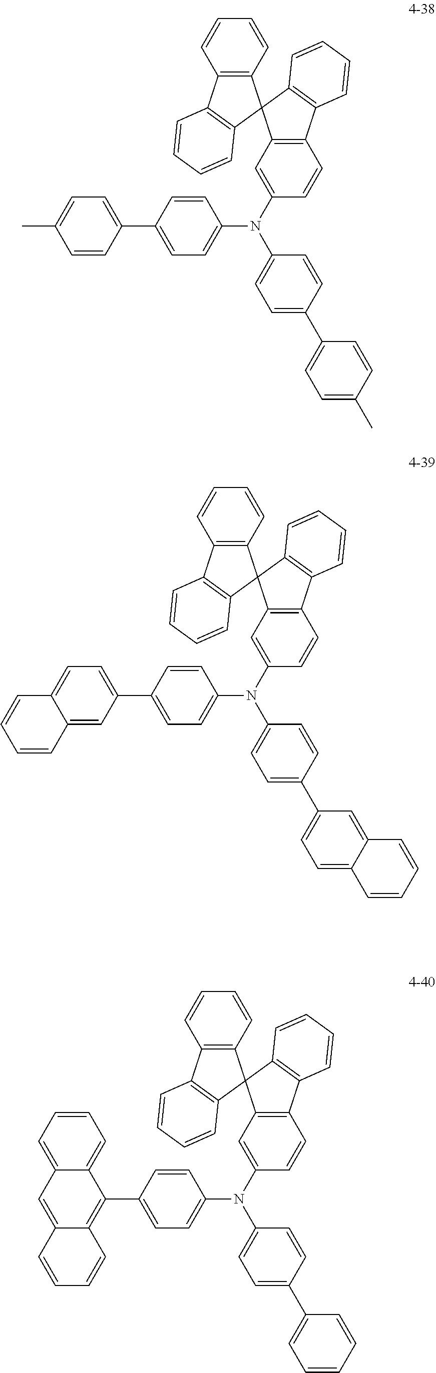

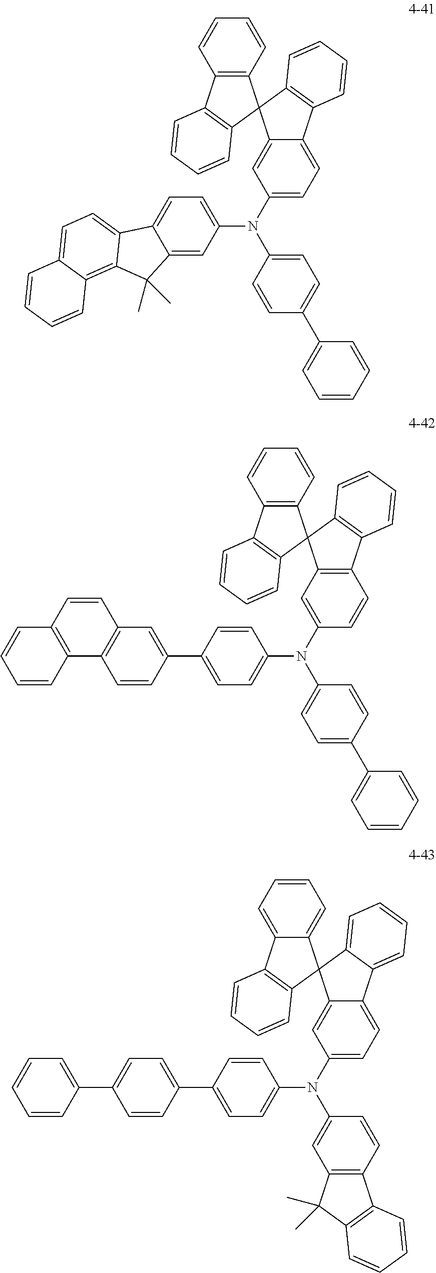

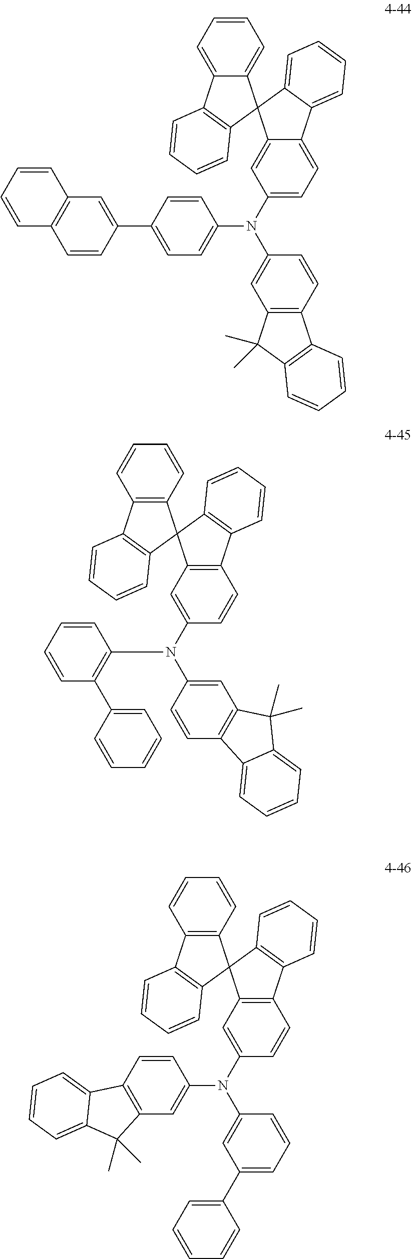

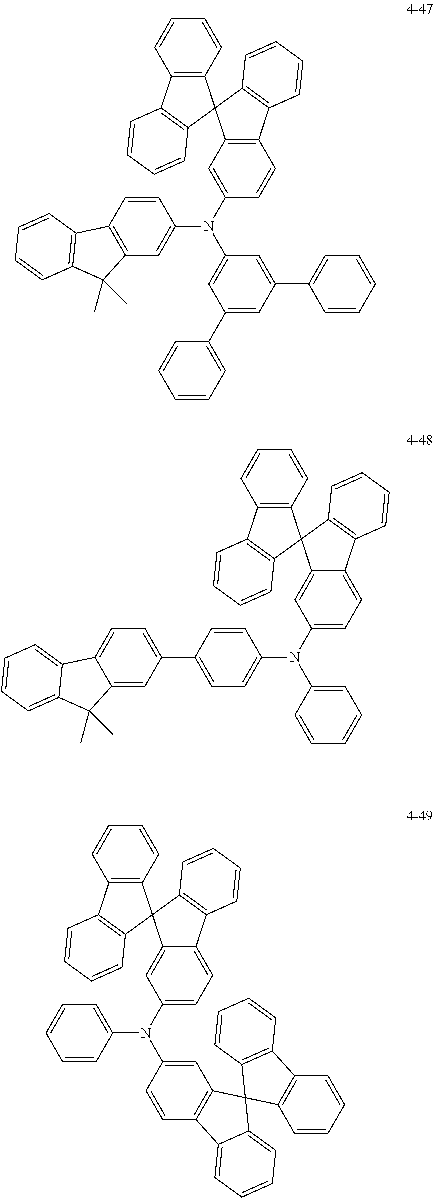

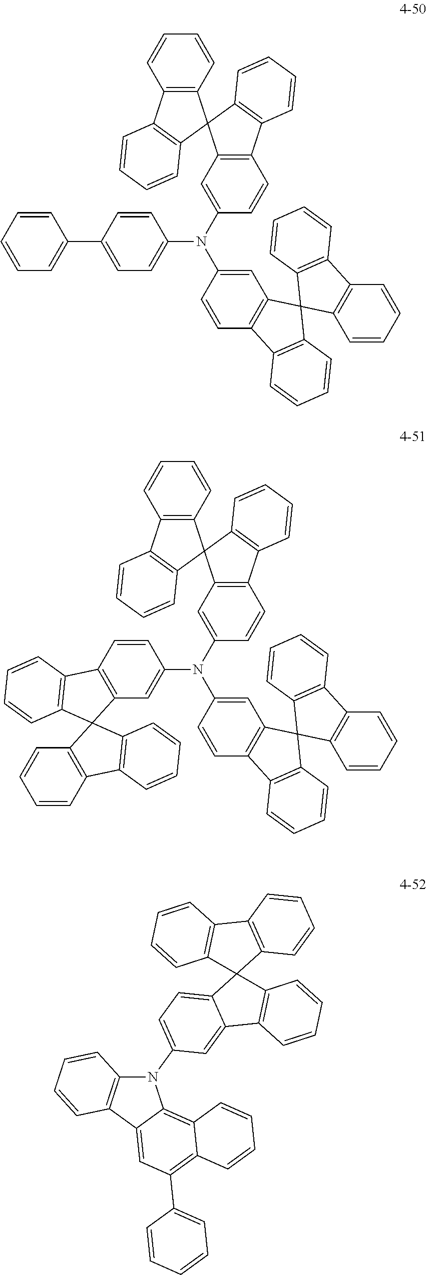

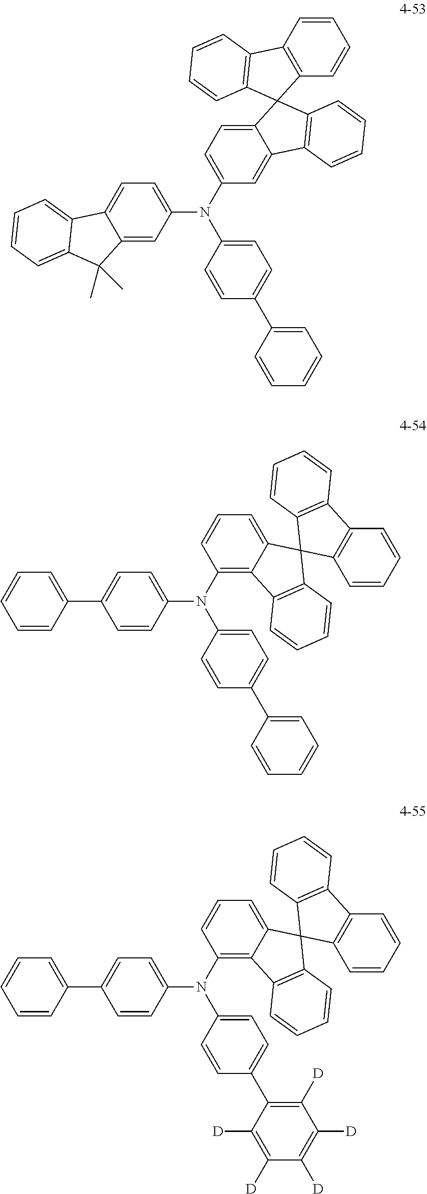

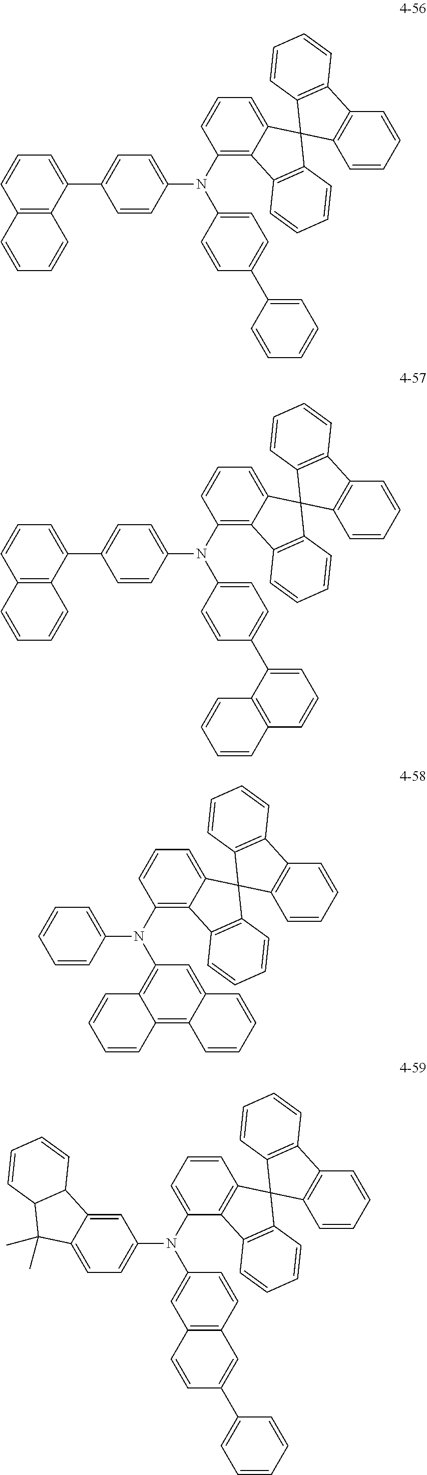

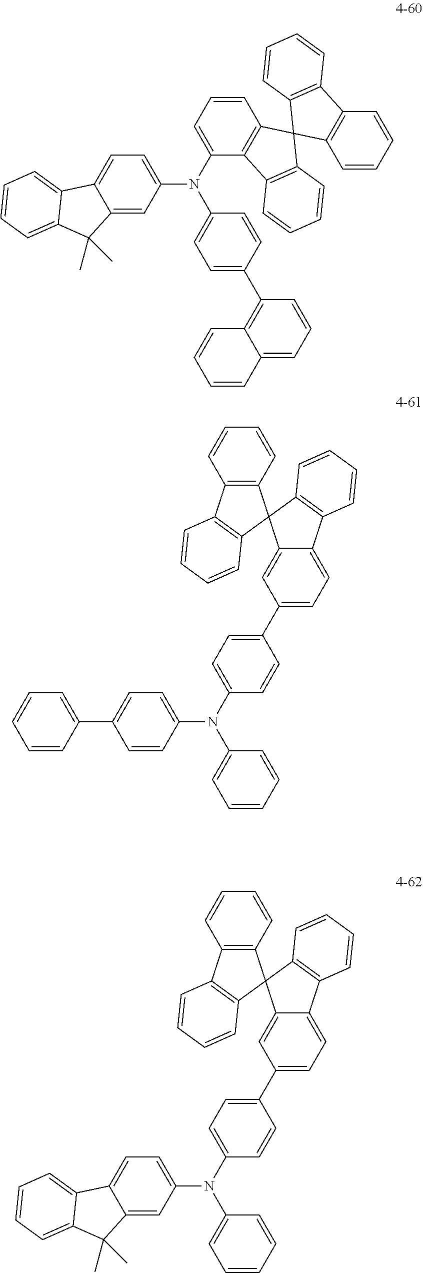

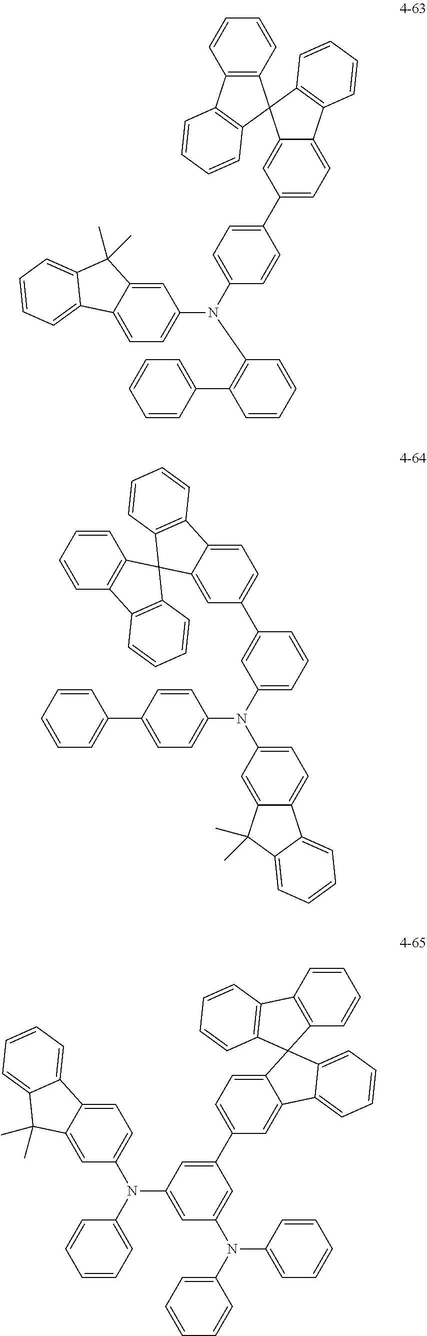

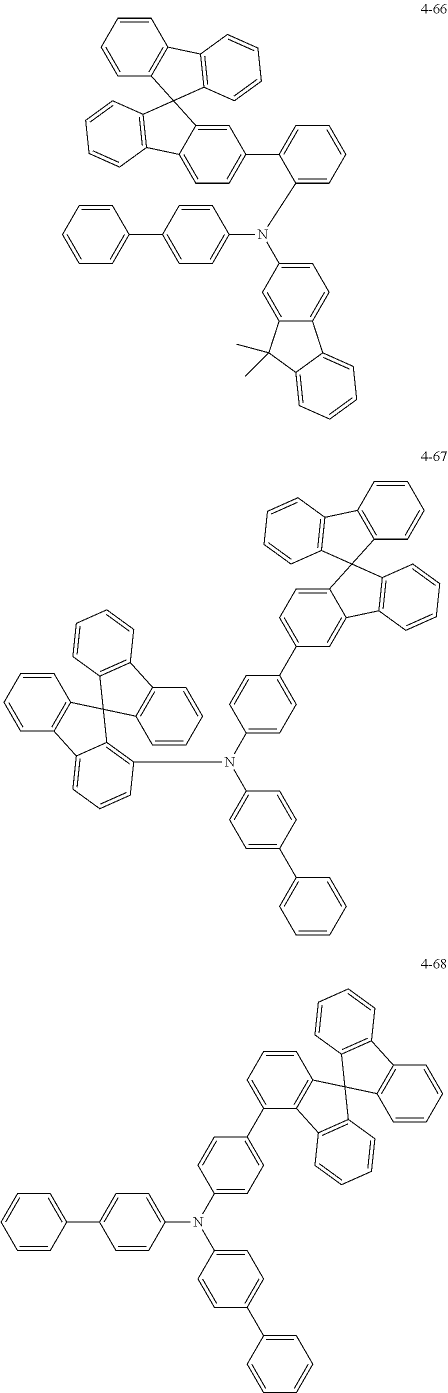

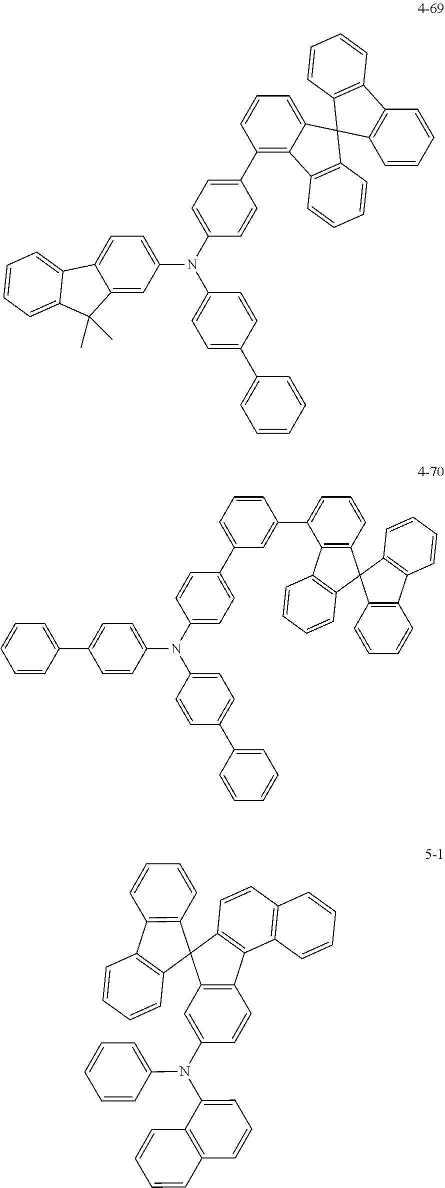

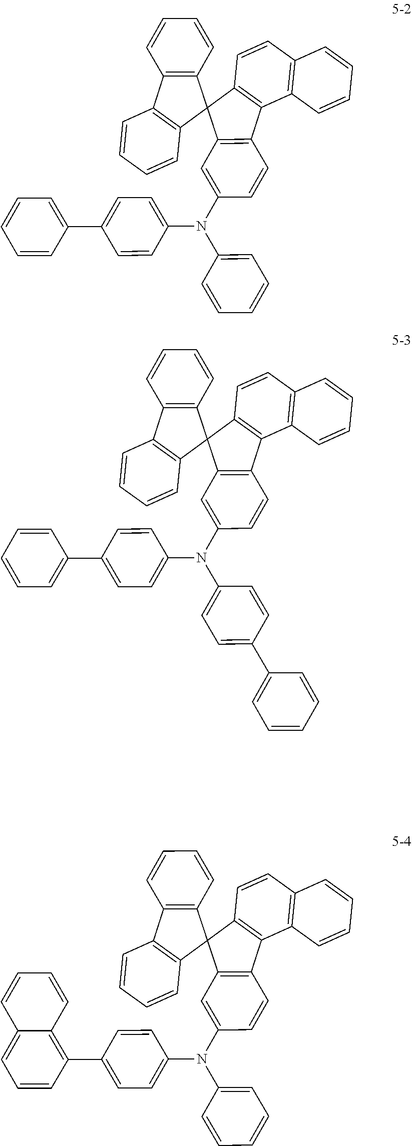

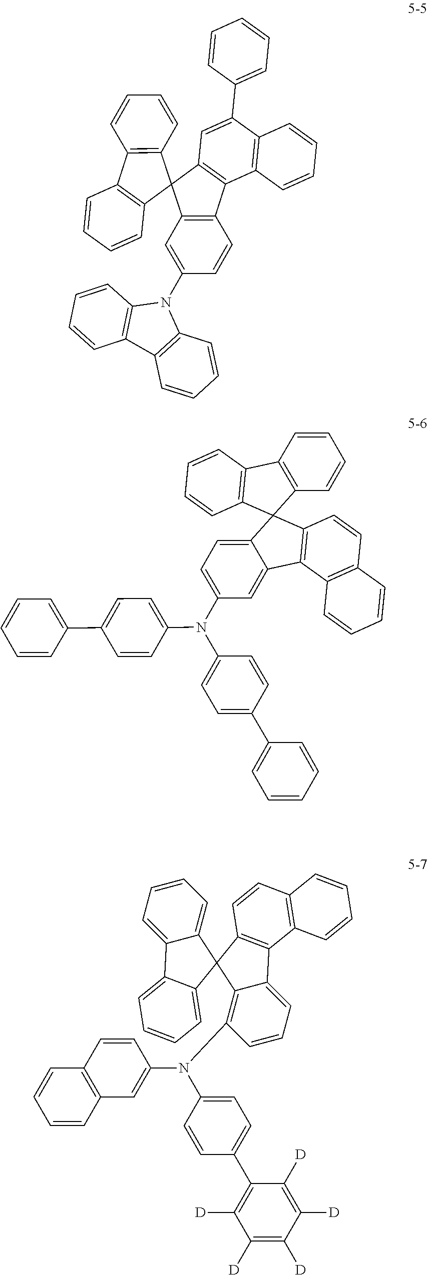

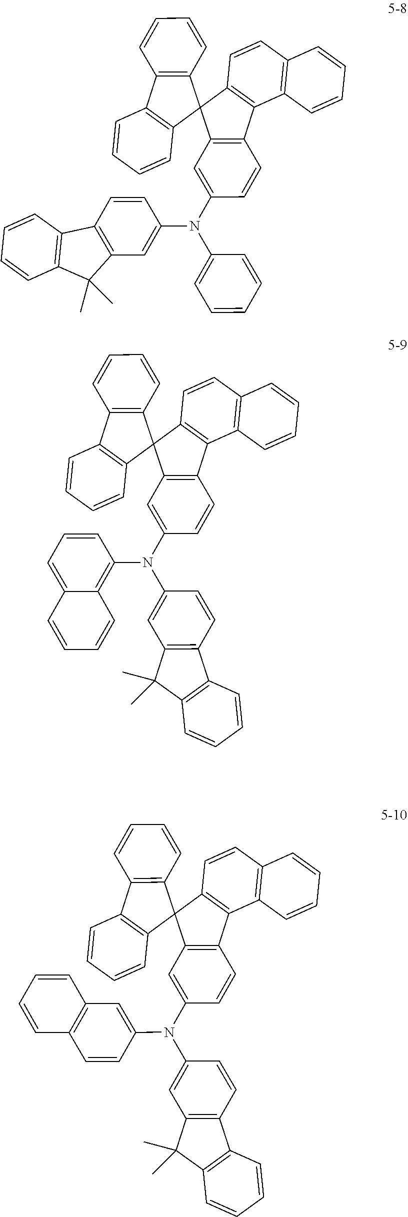

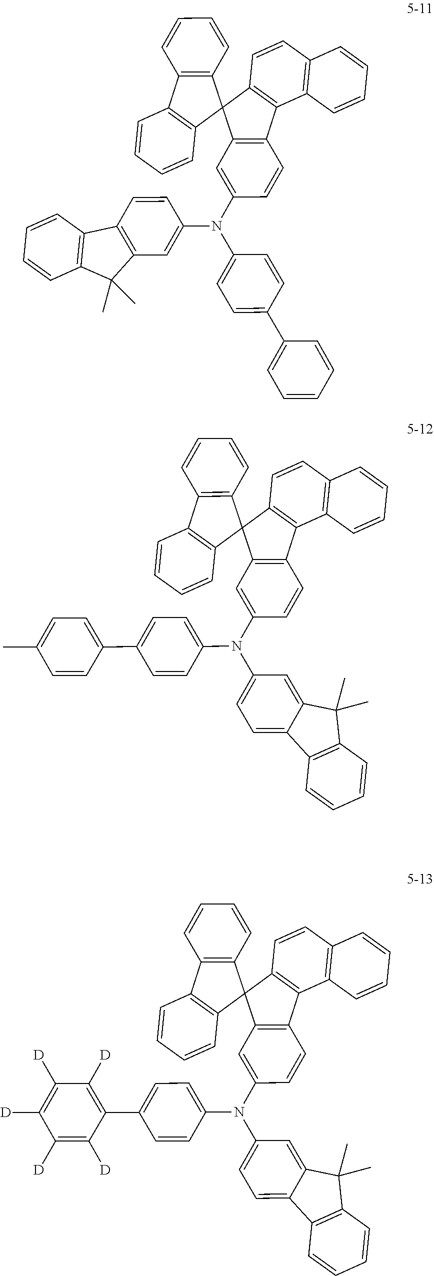

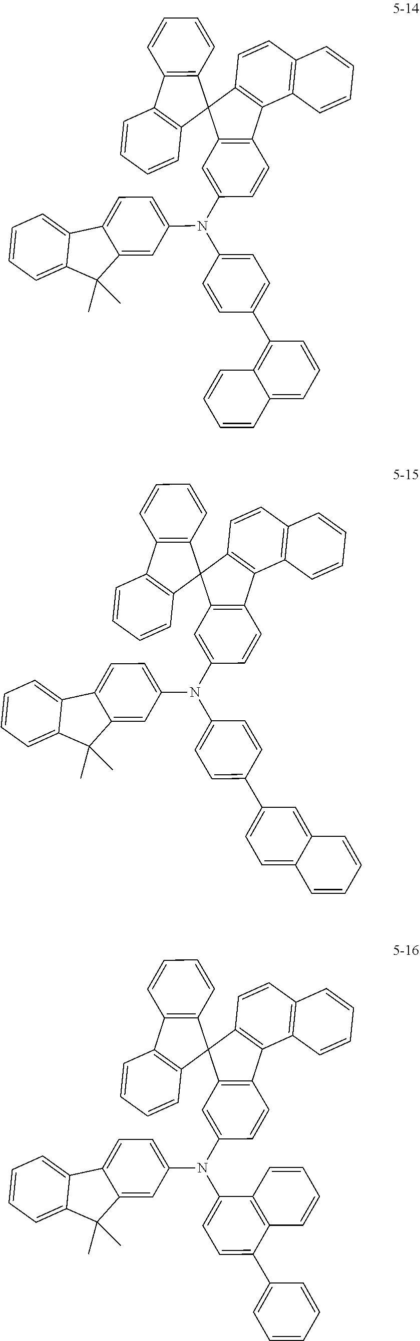

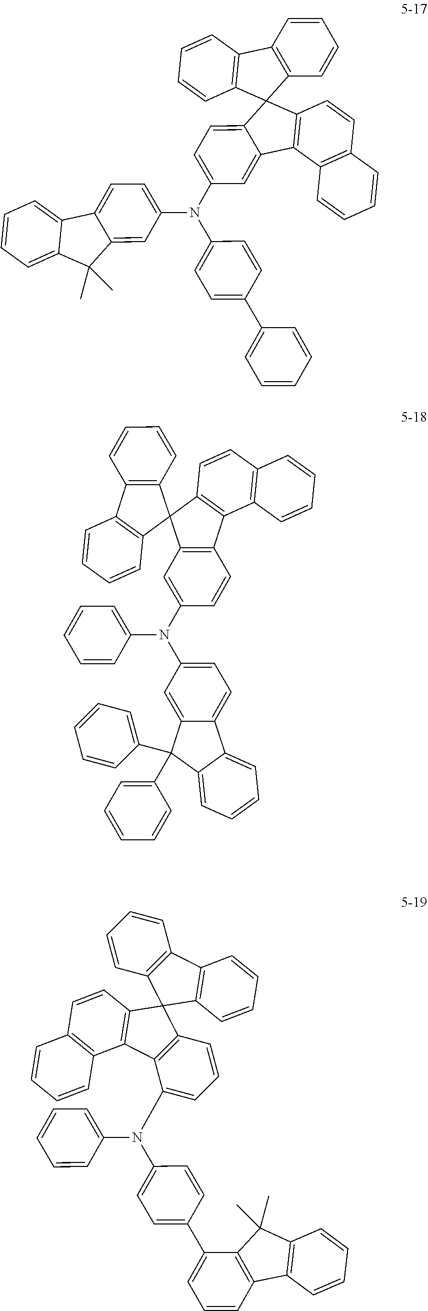

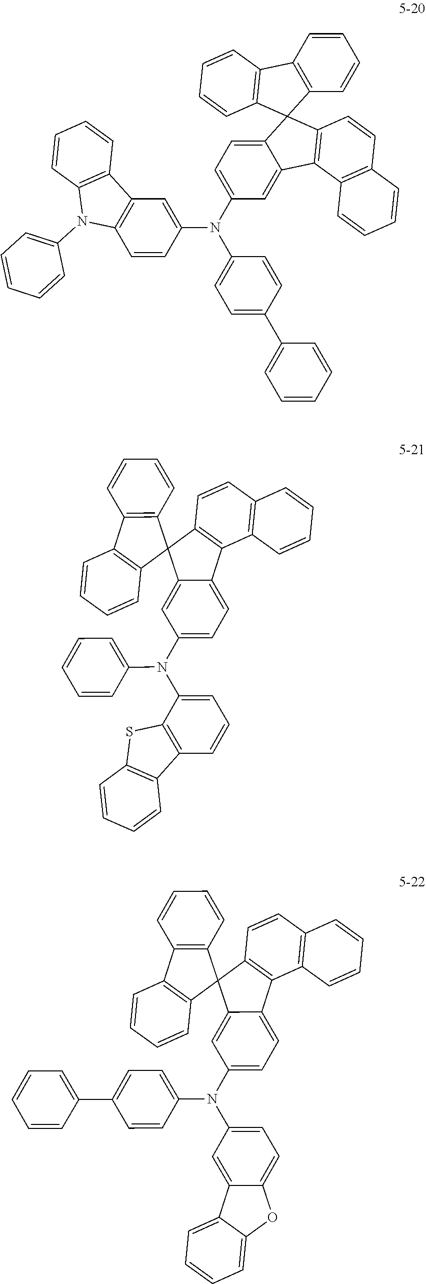

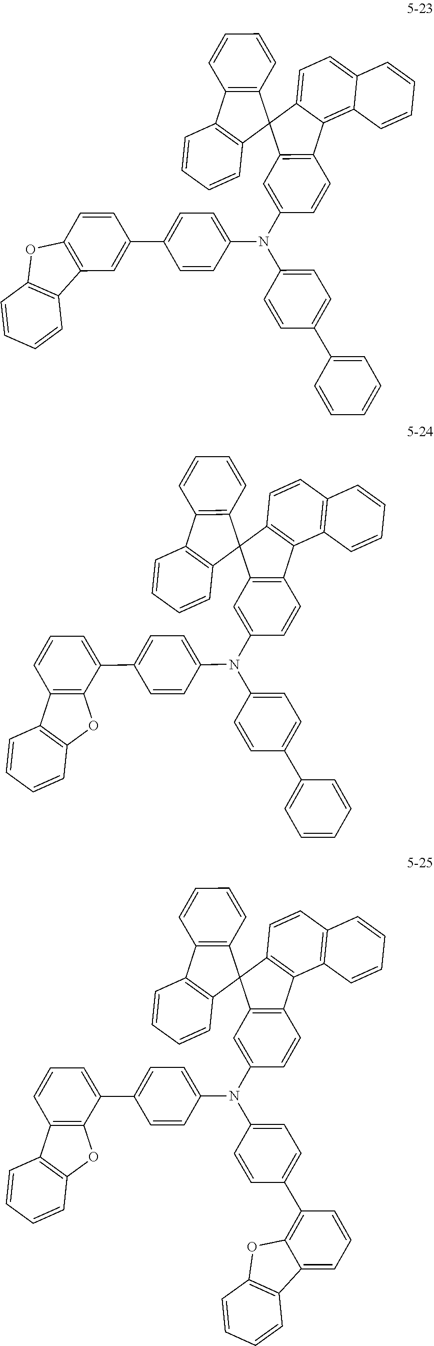

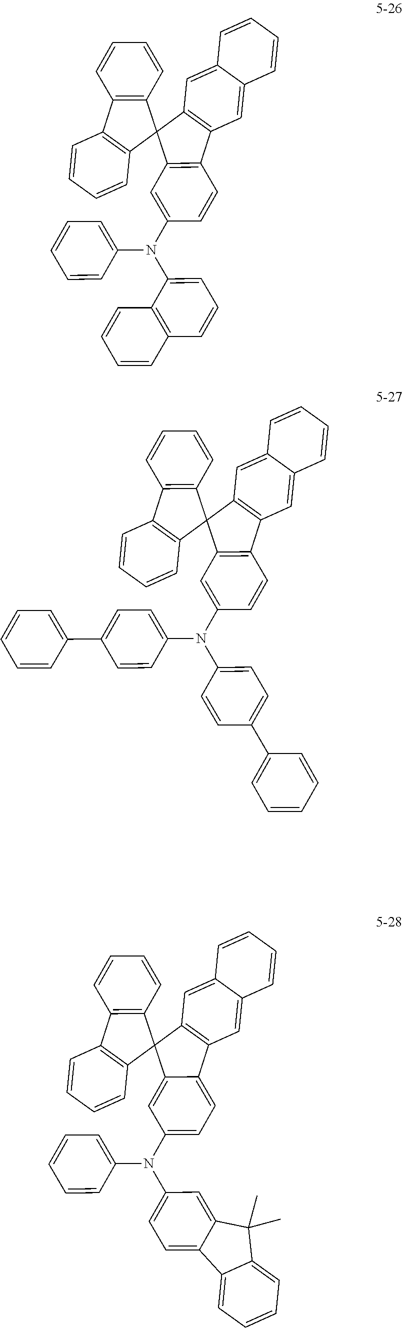

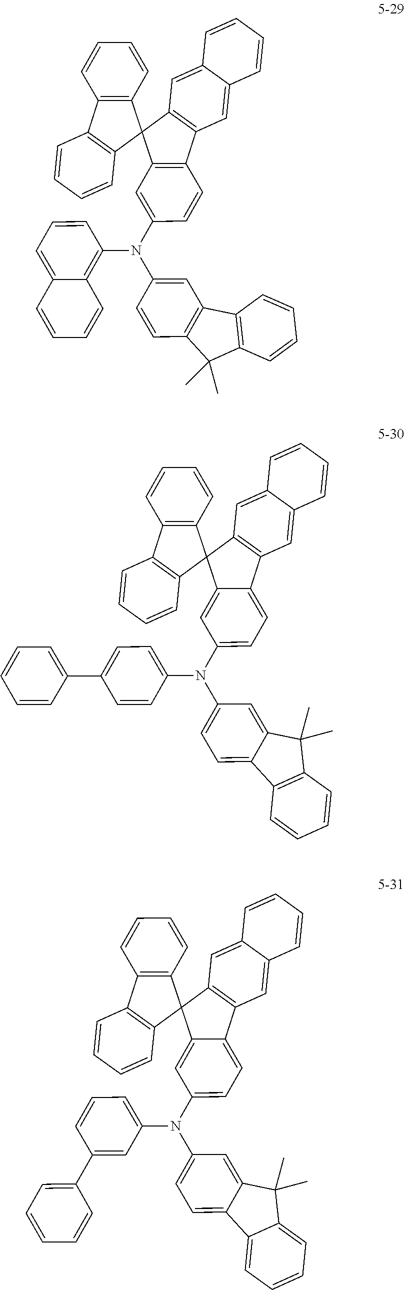

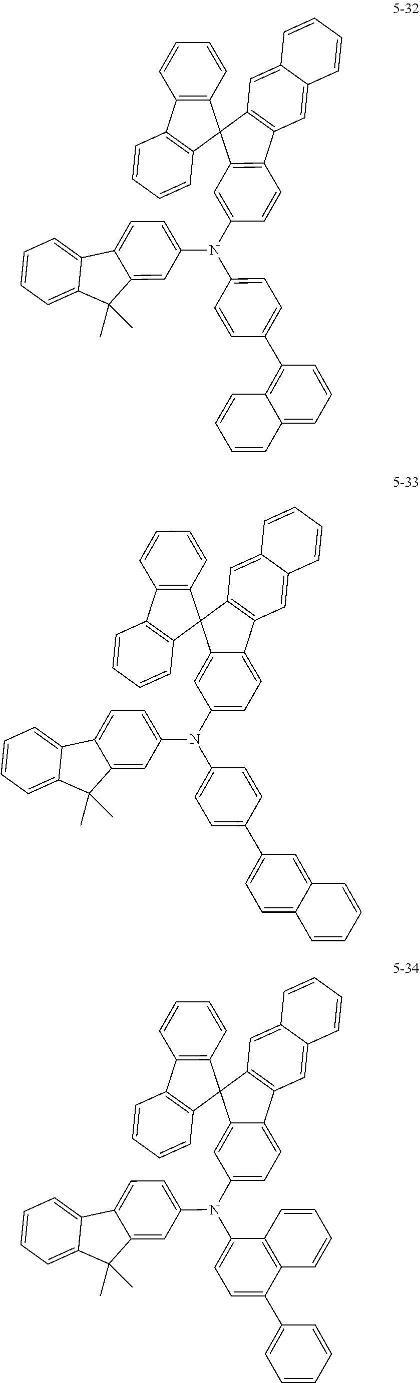

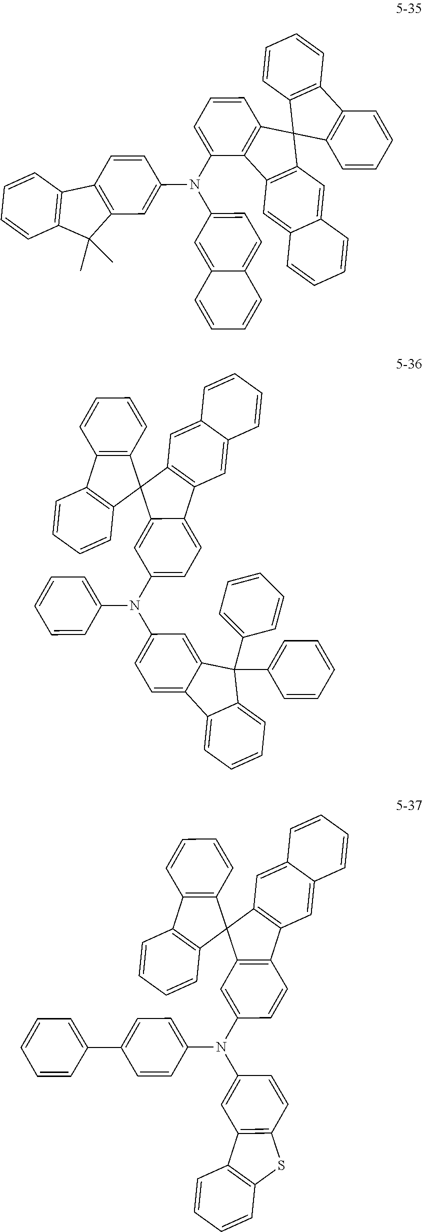

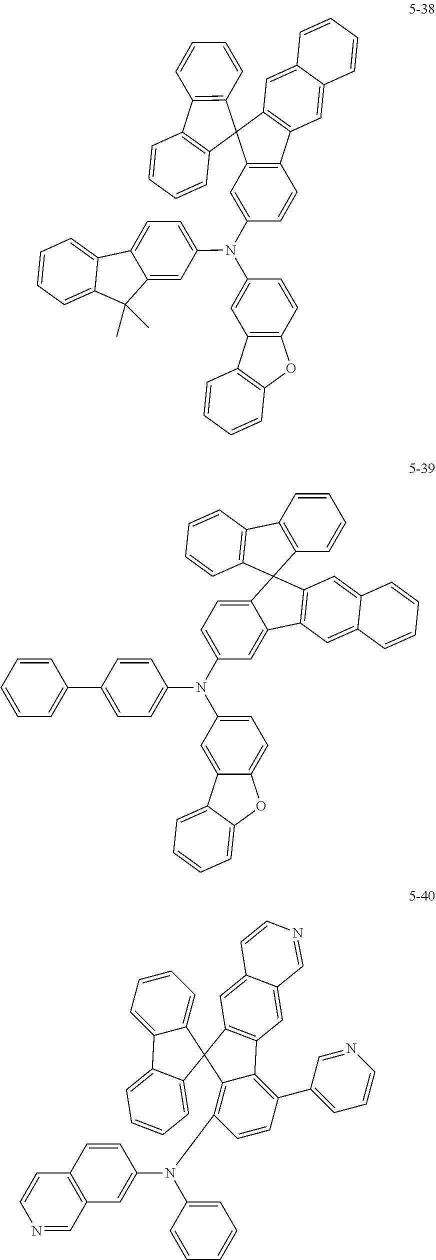

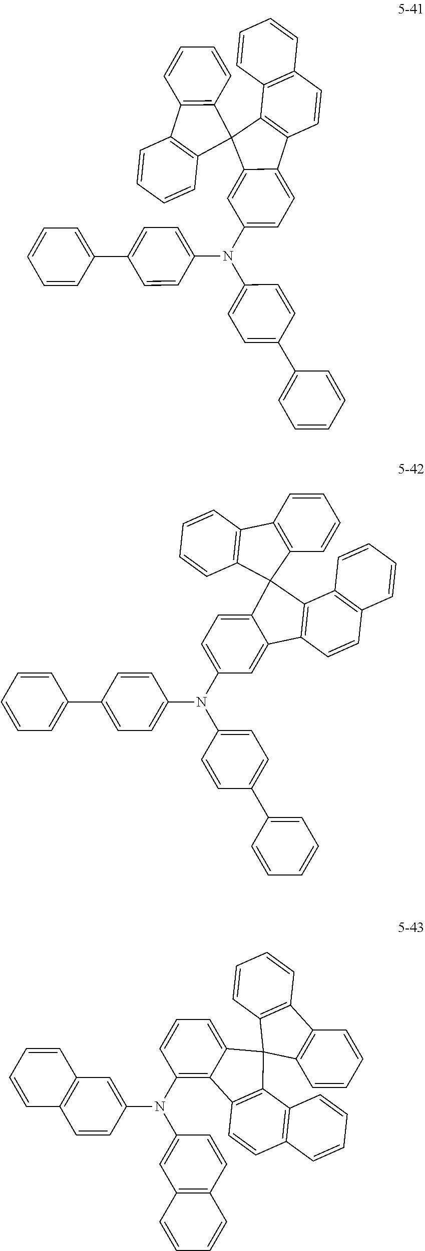

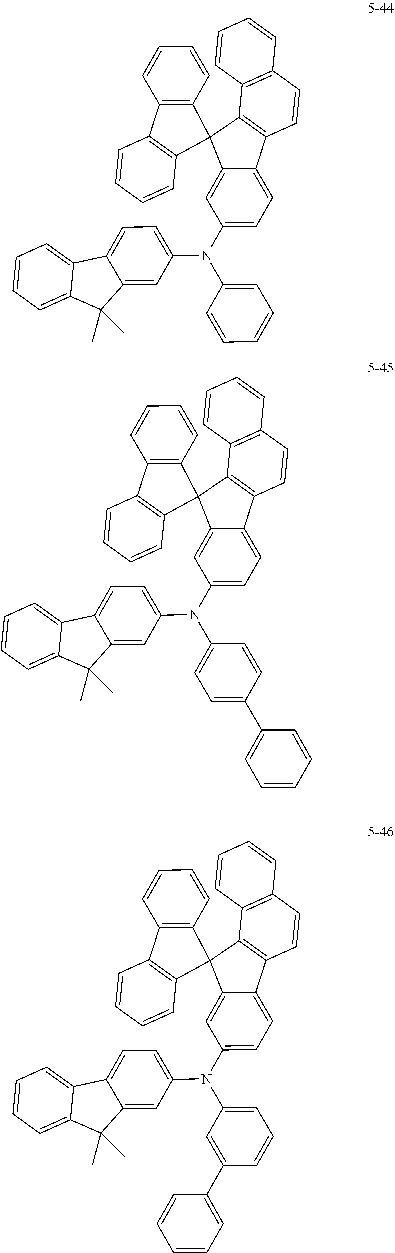

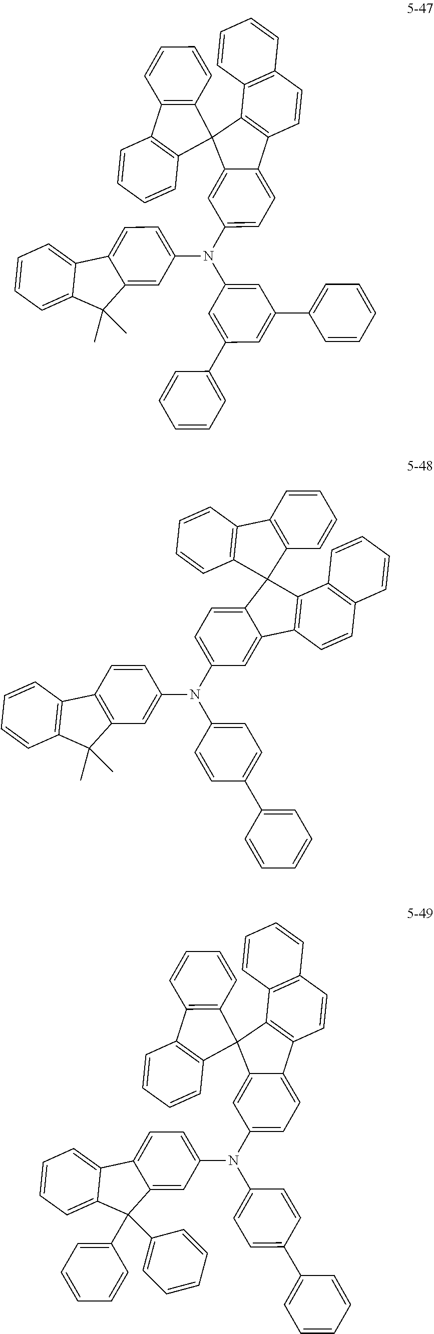

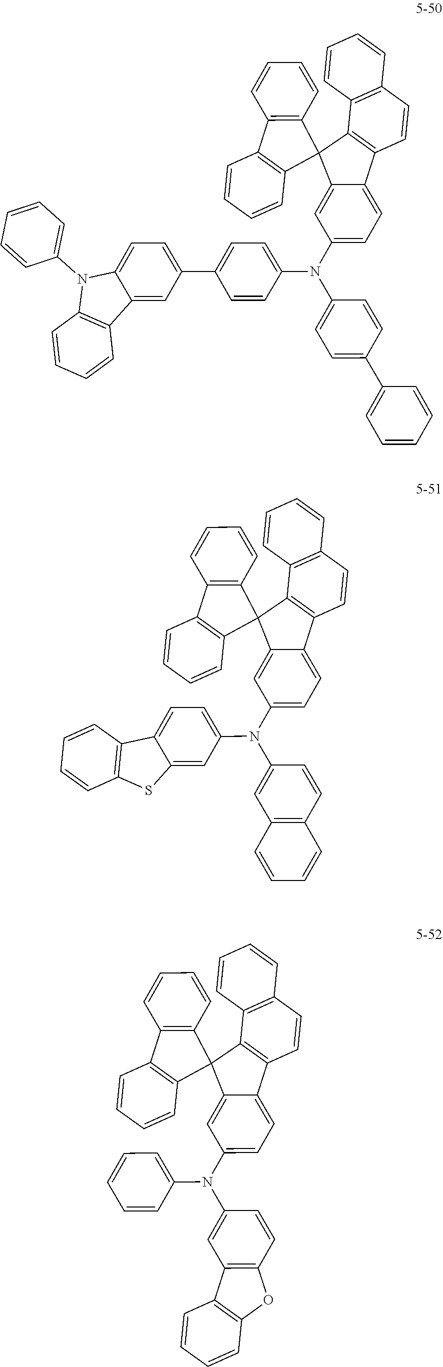

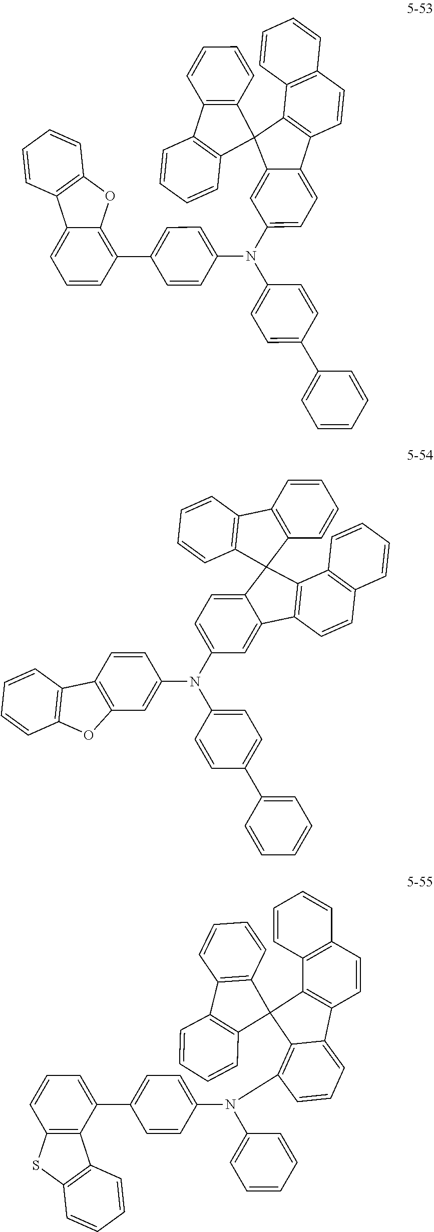

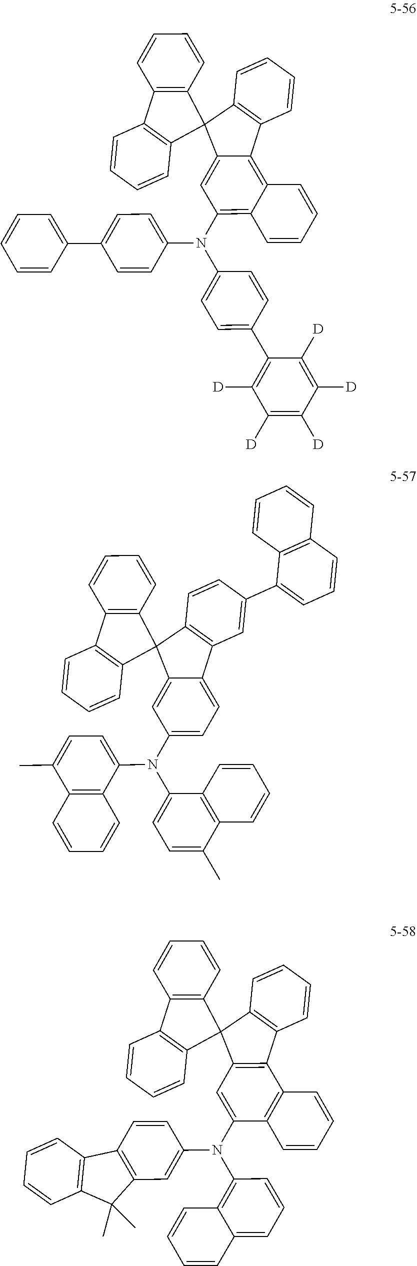

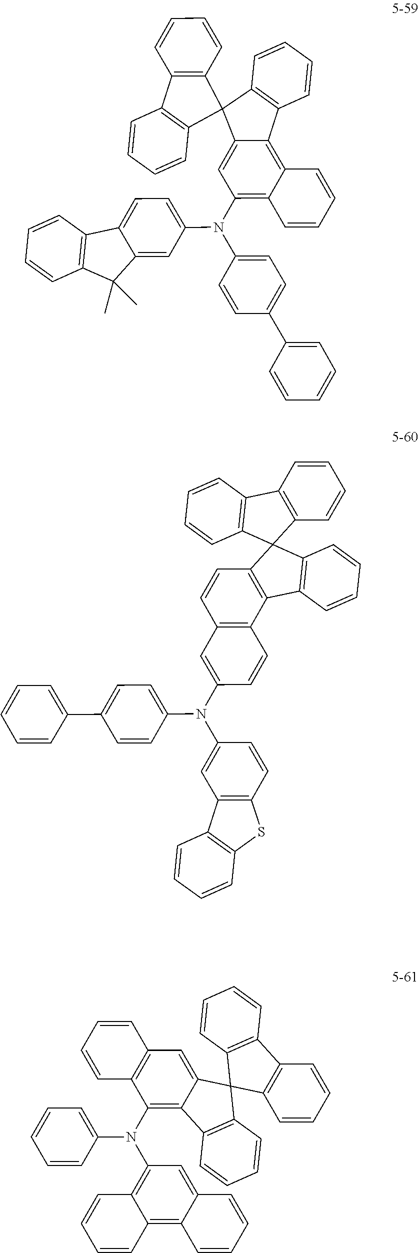

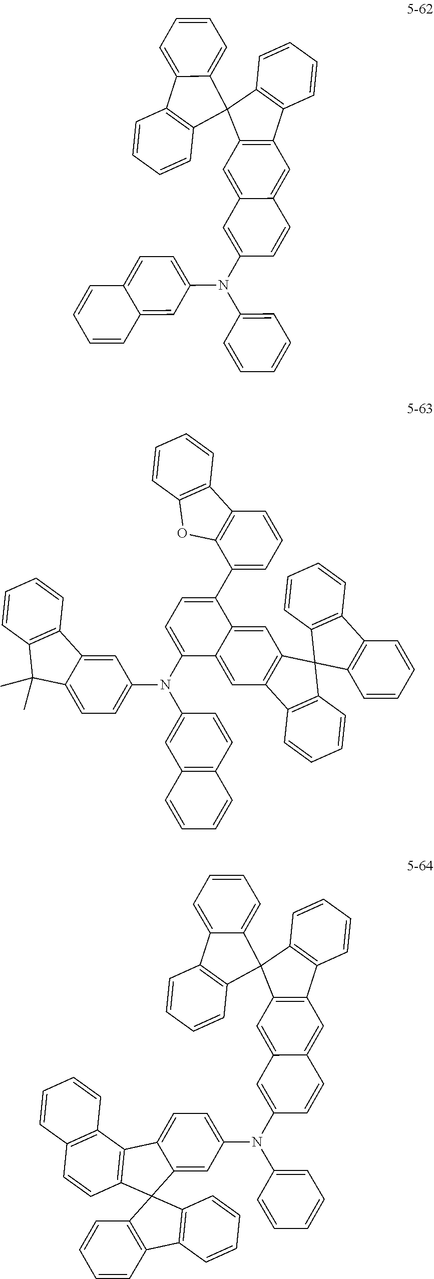

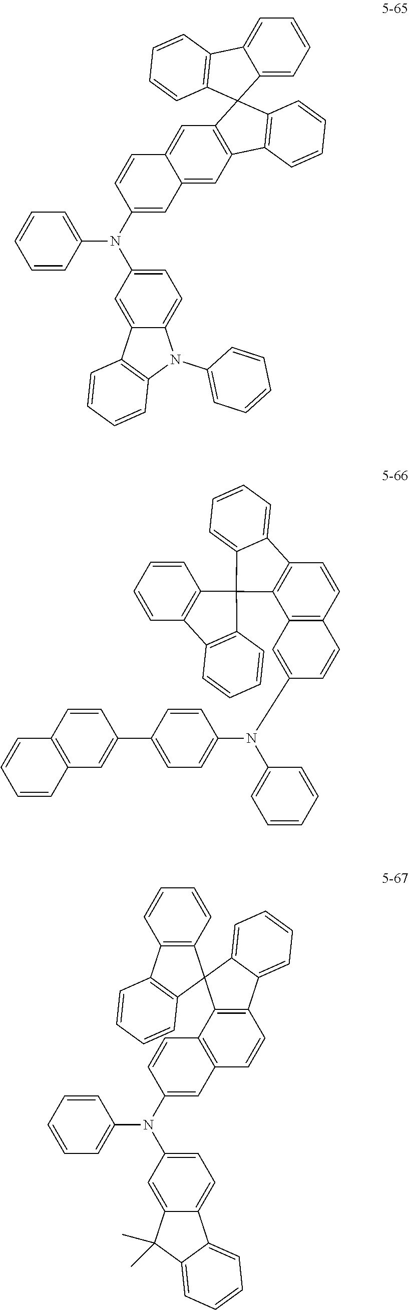

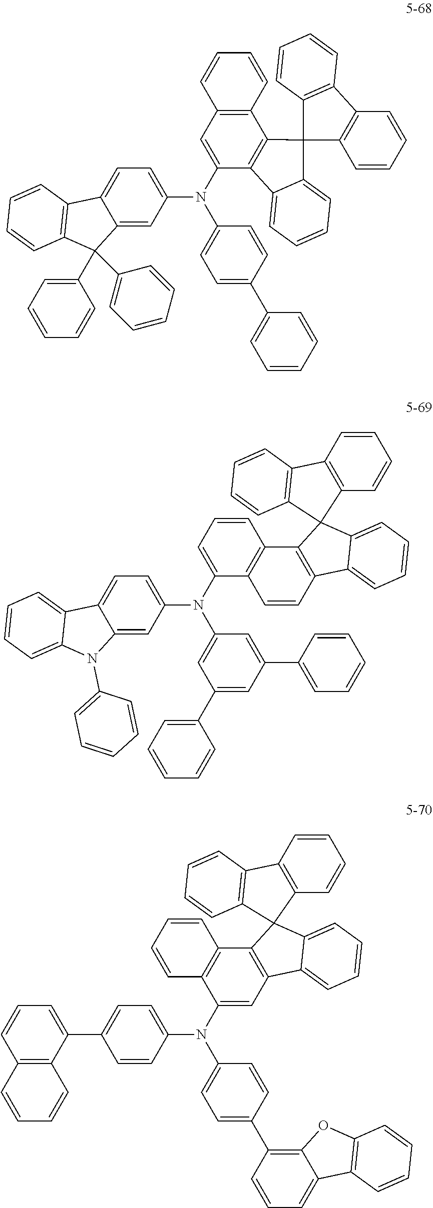

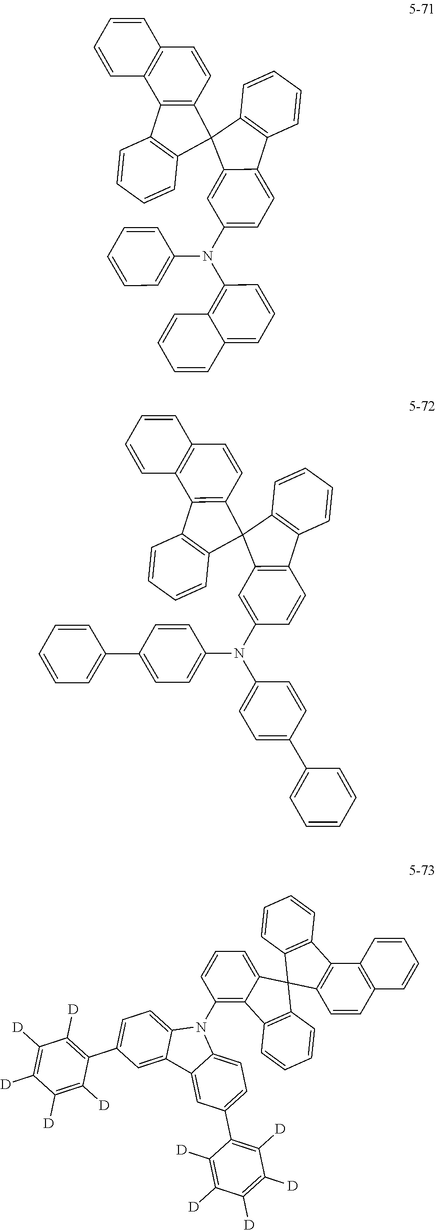

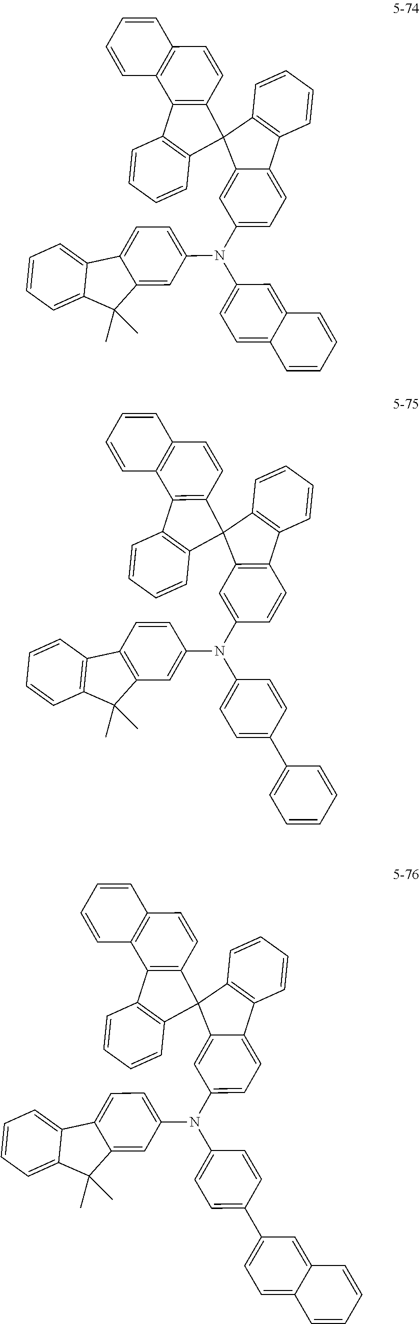

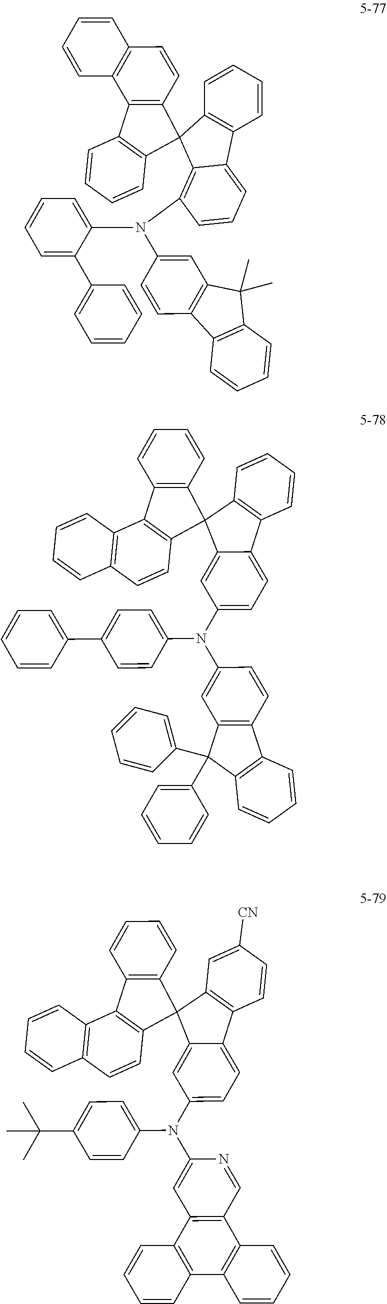

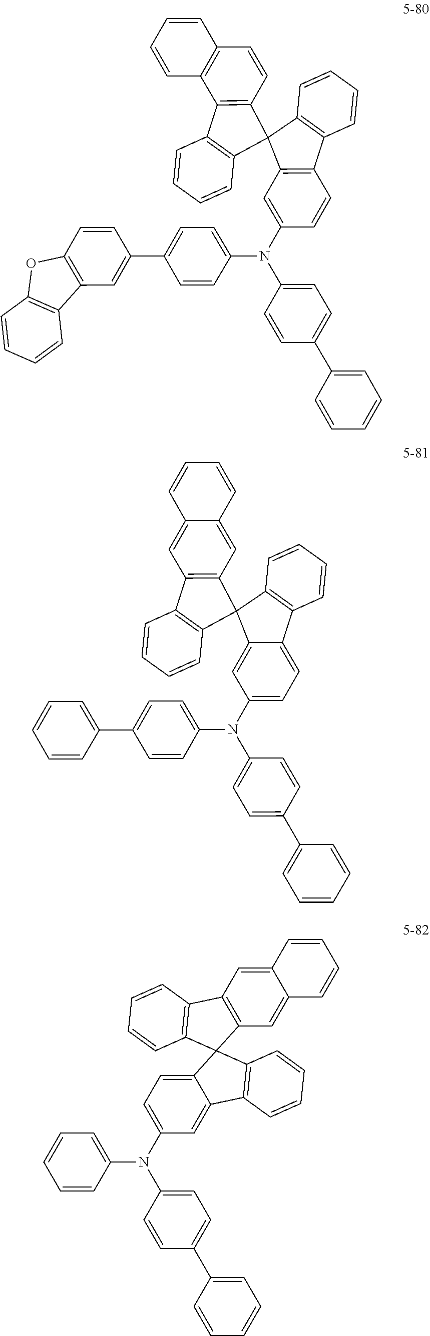

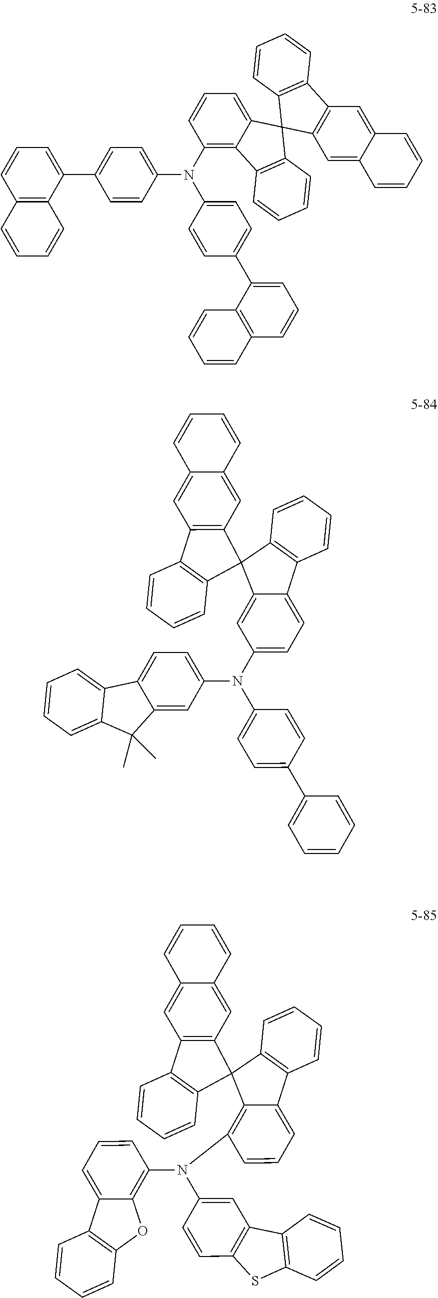

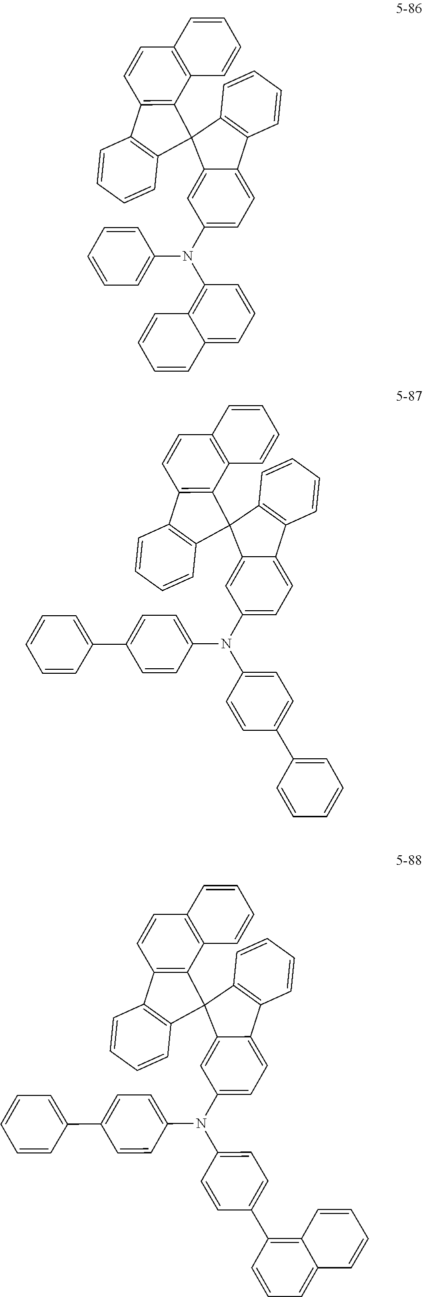

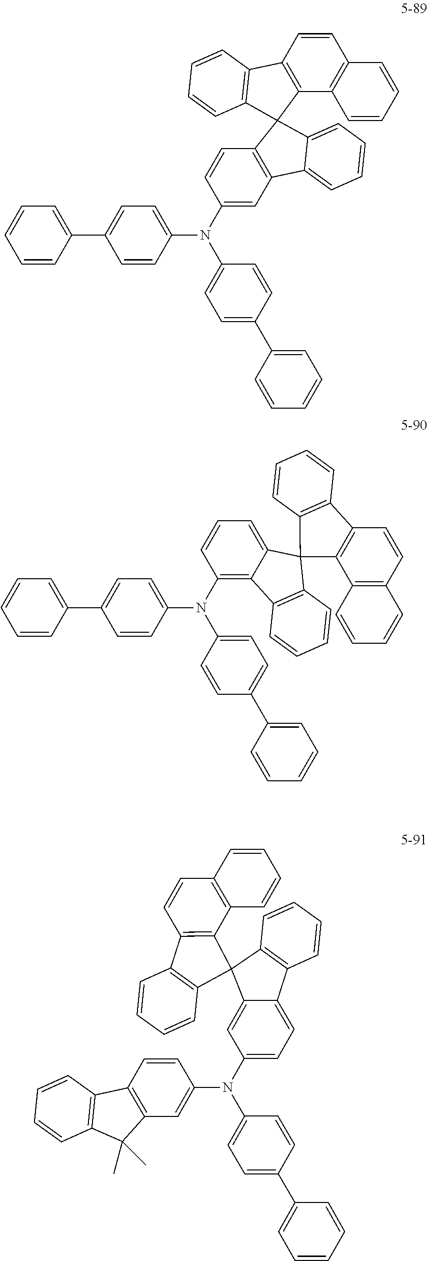

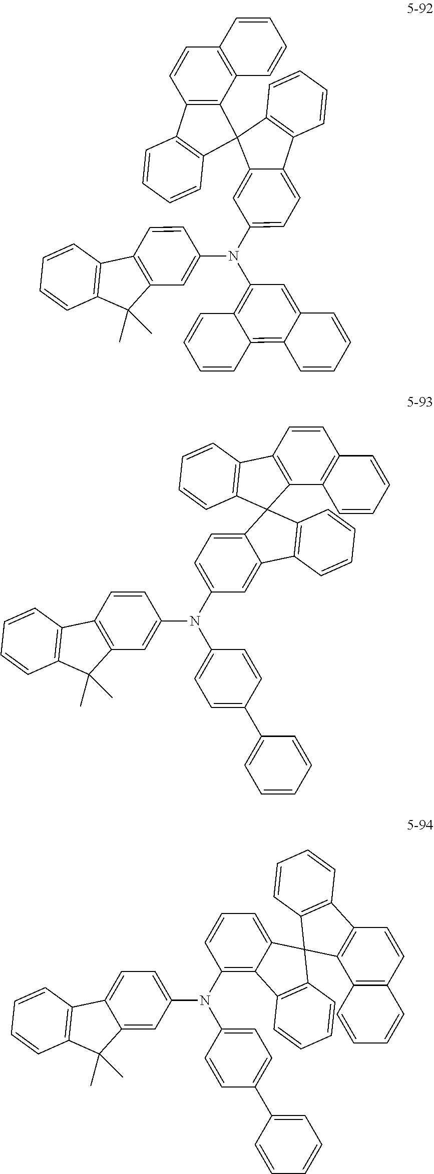

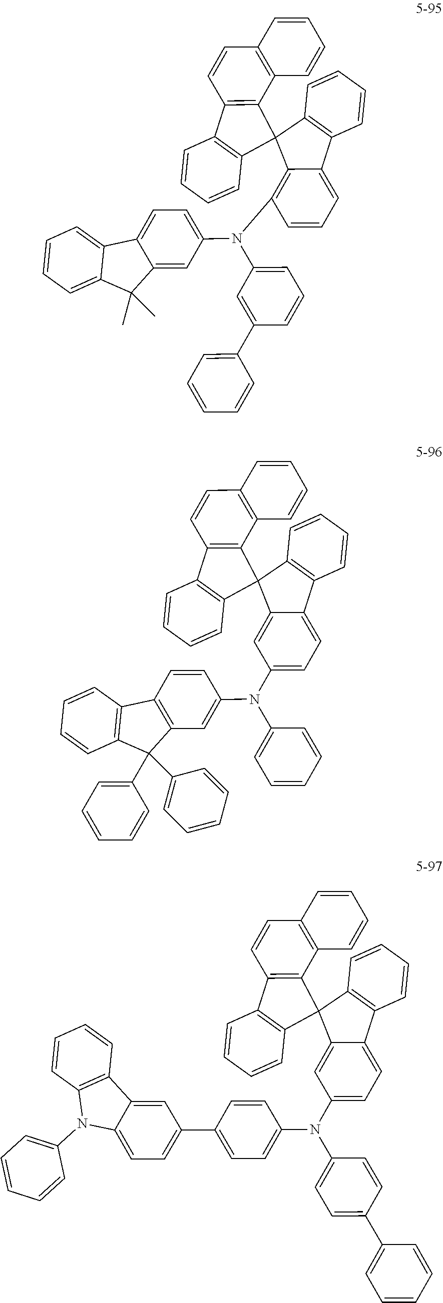

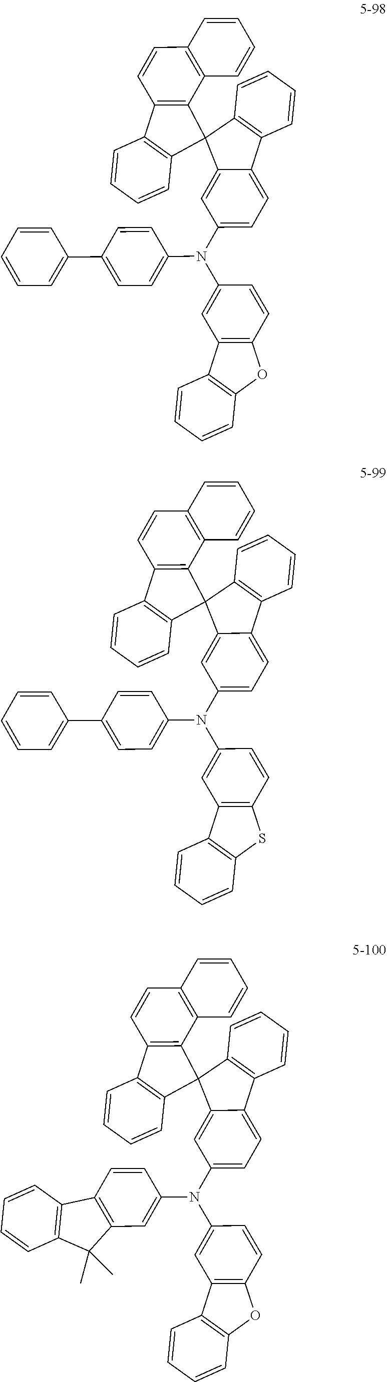

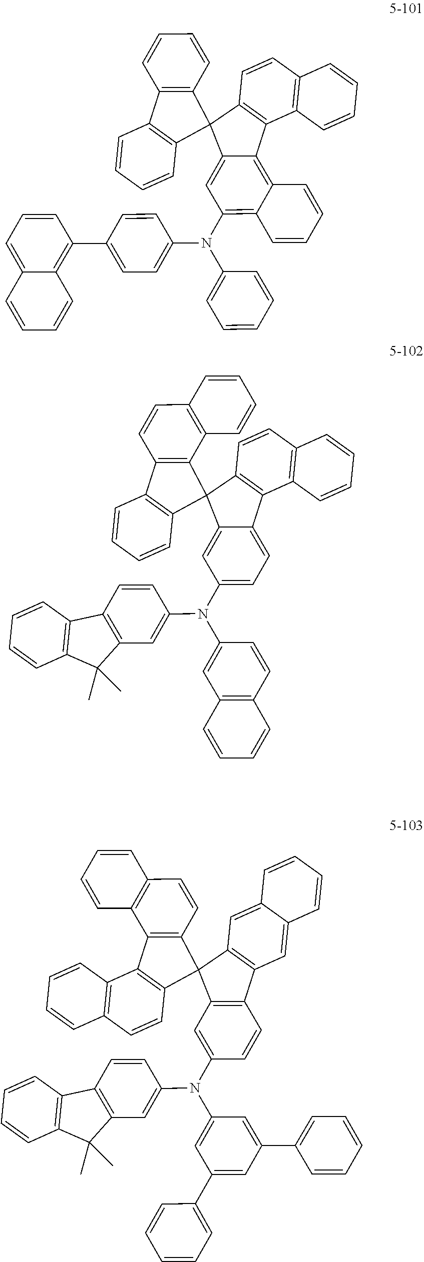

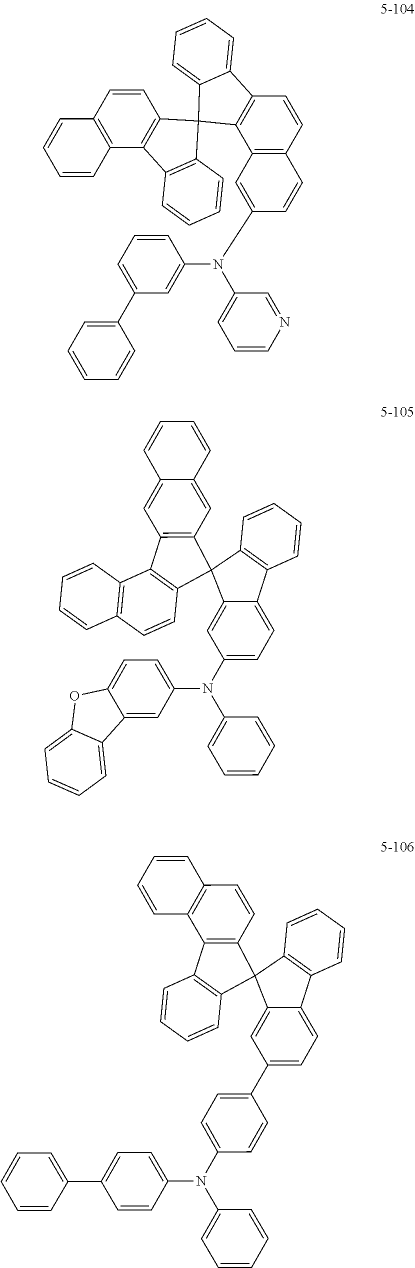

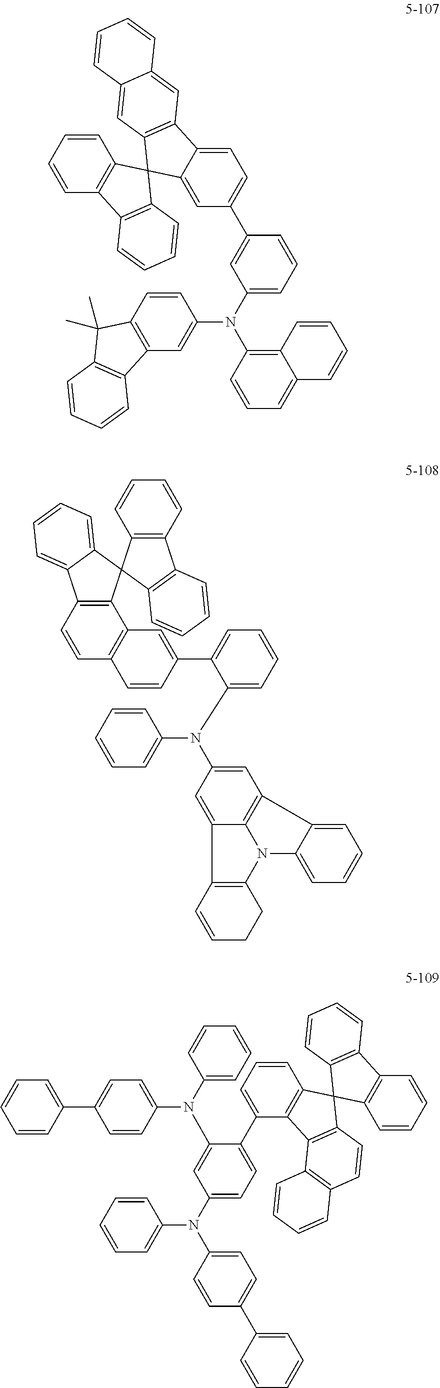

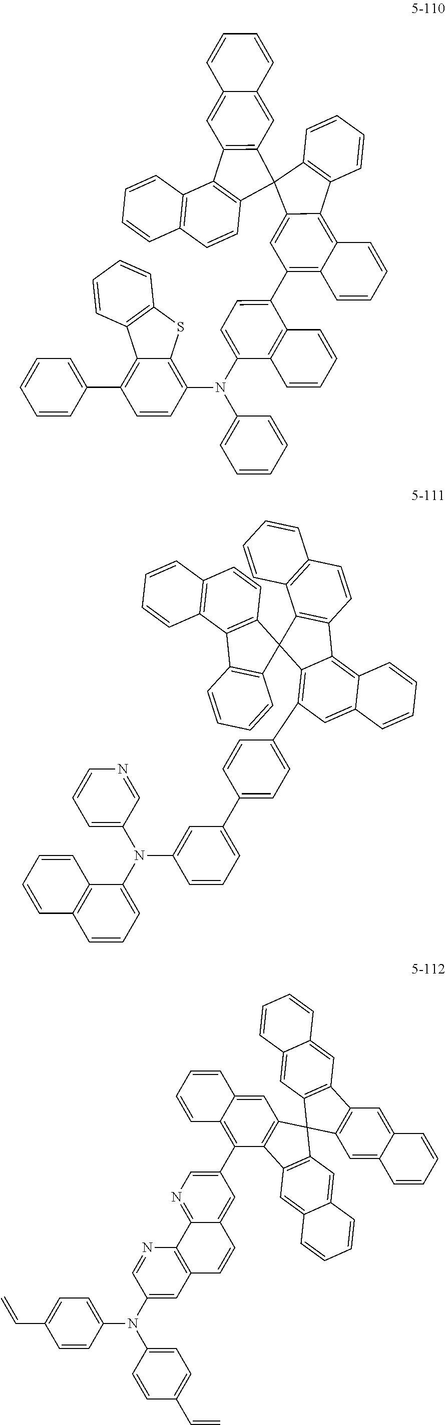

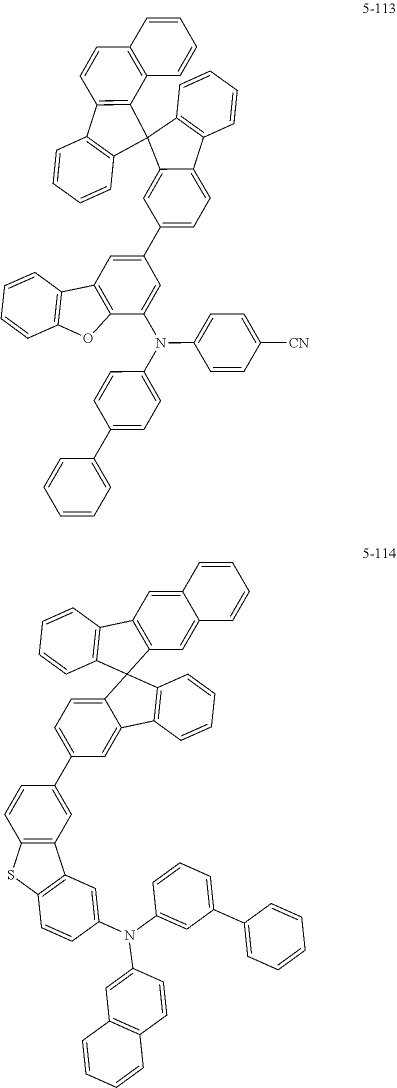

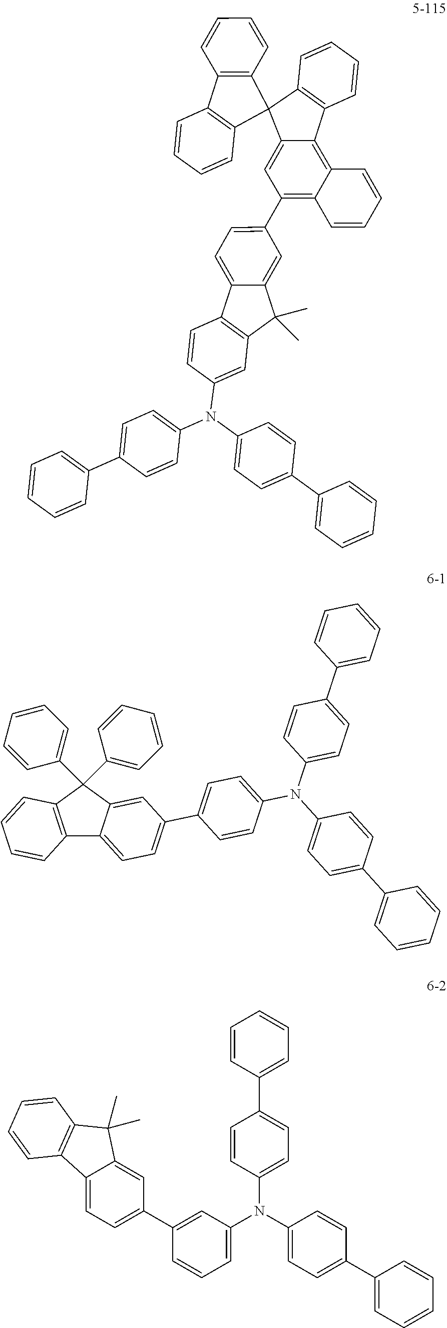

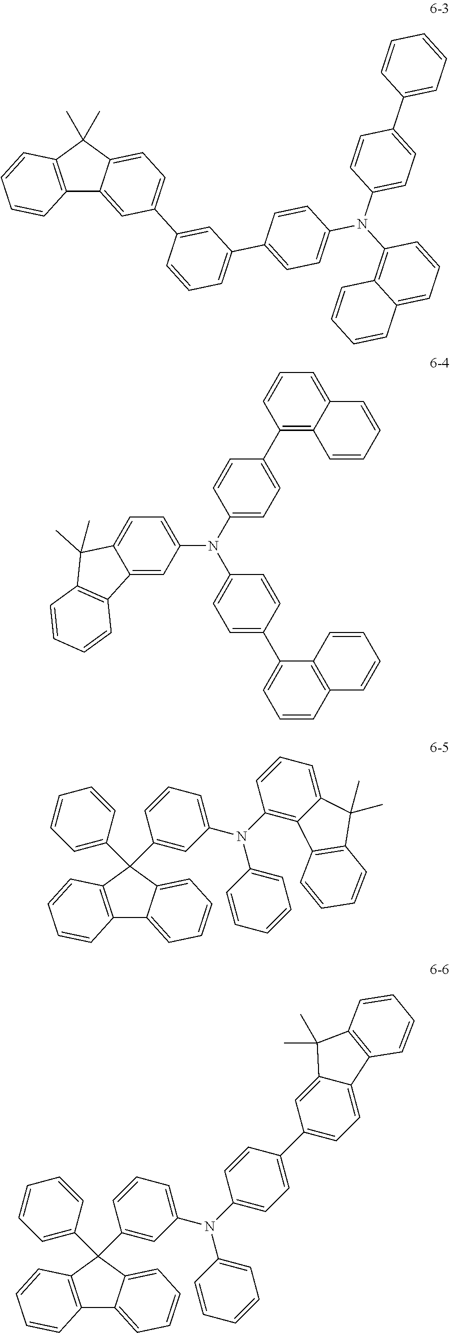

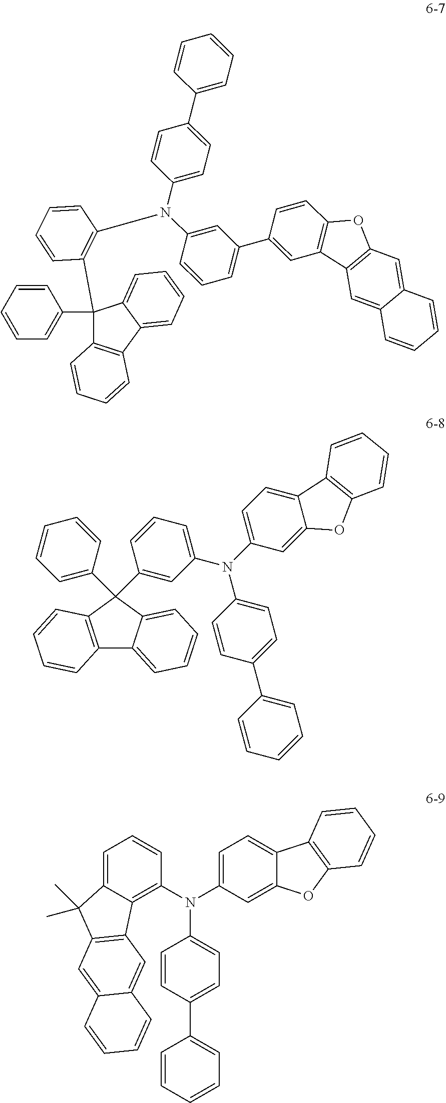

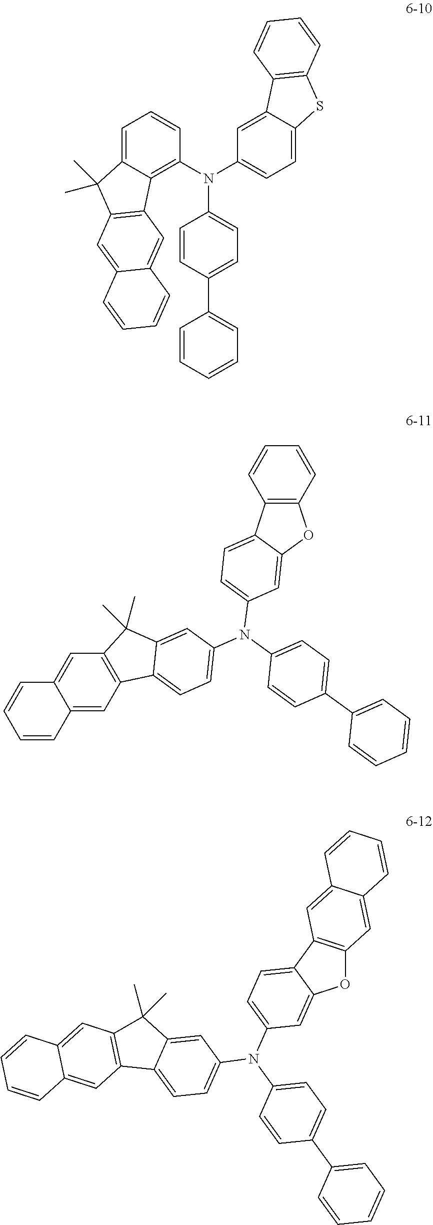

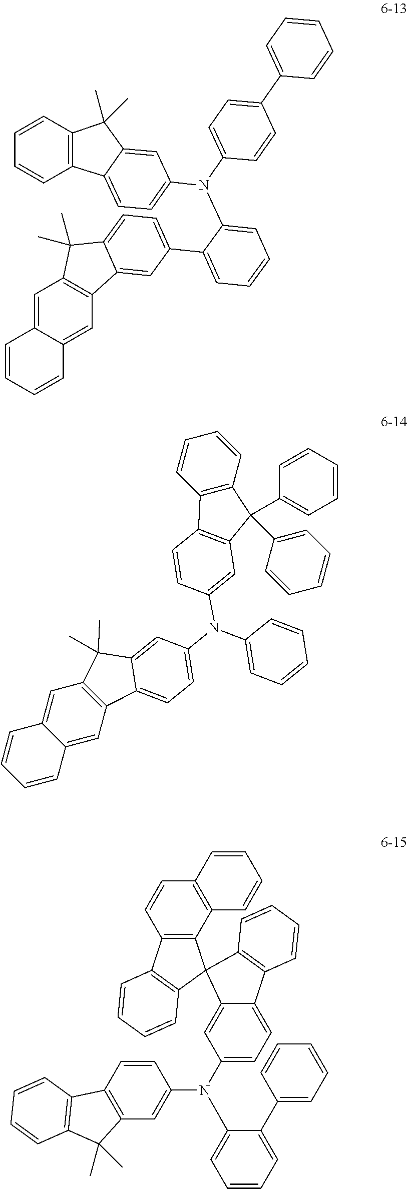

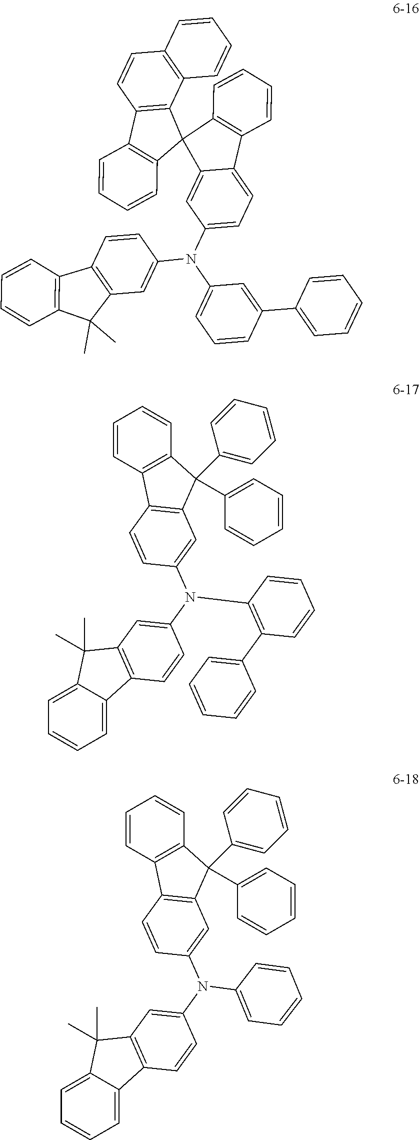

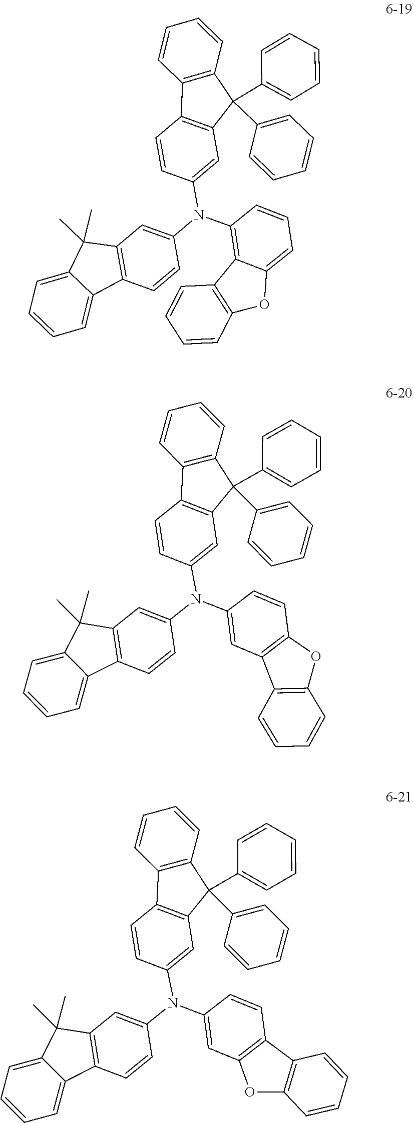

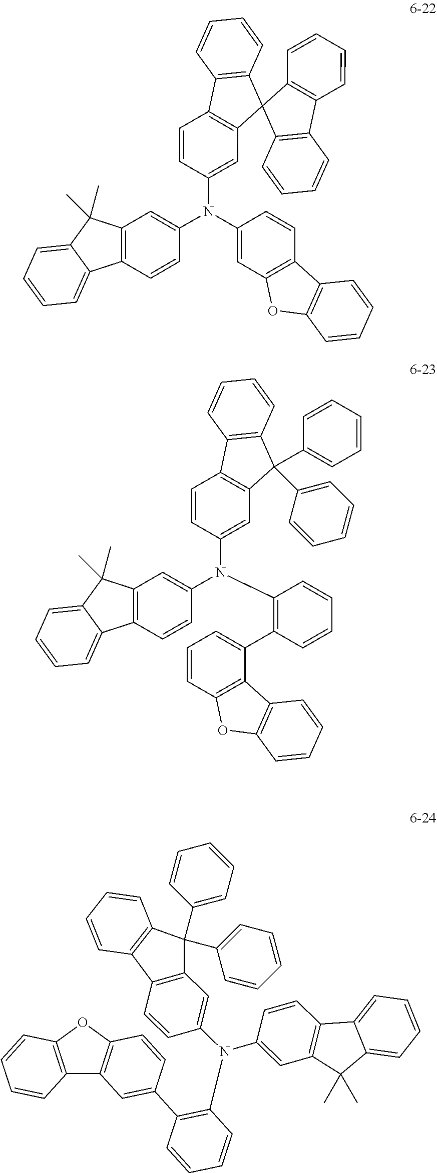

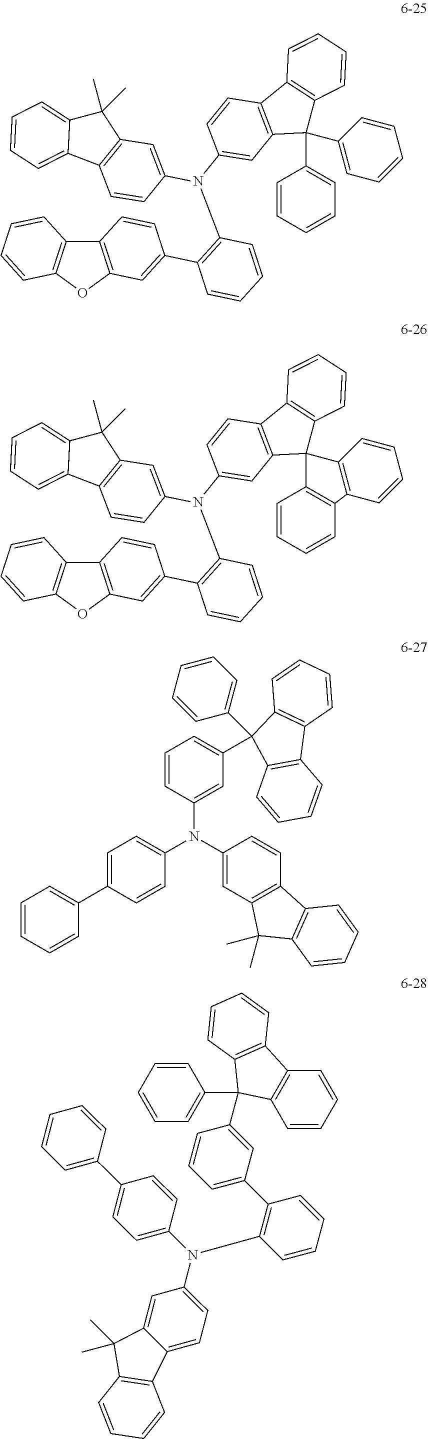

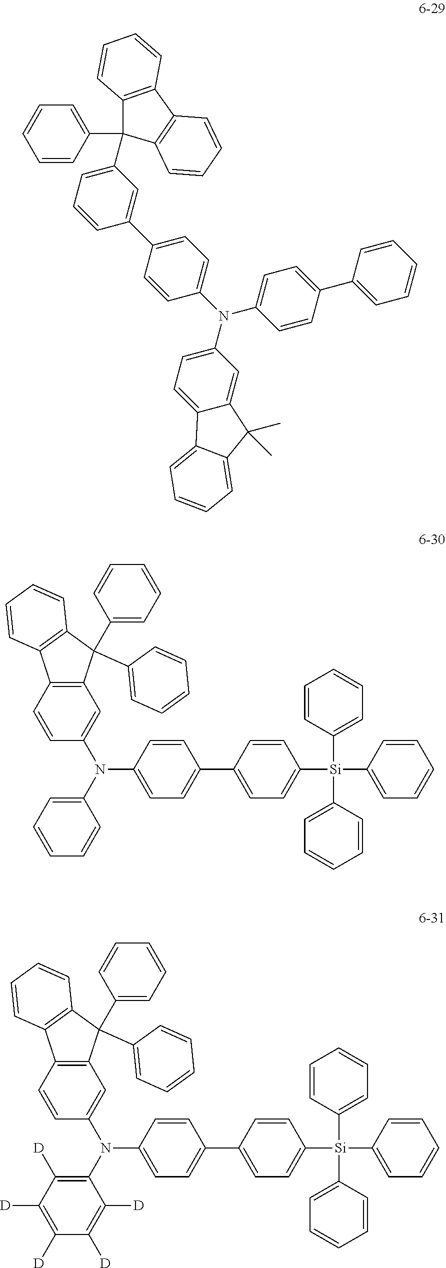

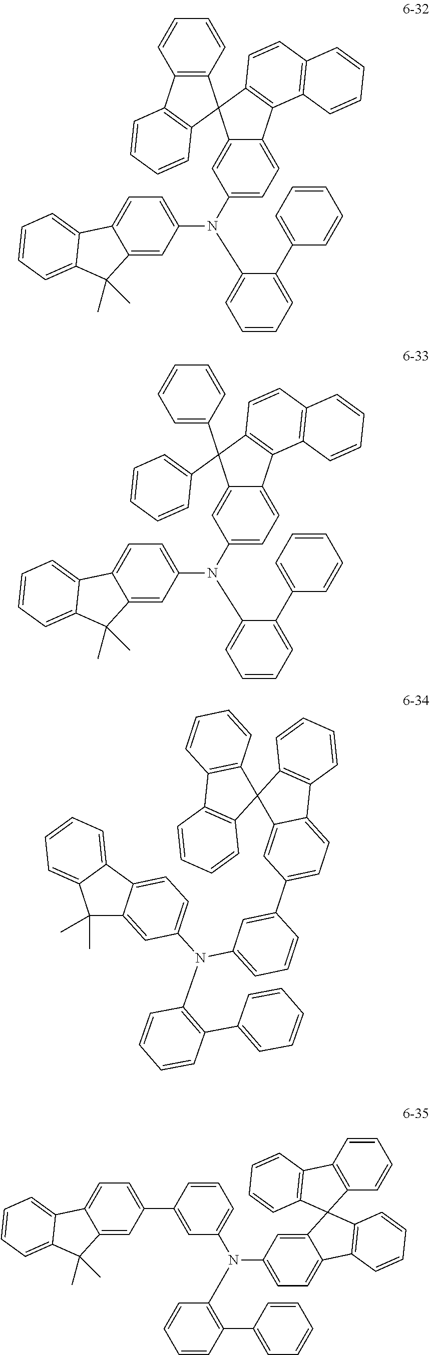

The fourth compound may include a radical of a compound represented by Formula A, be represented by Formula D, and be one of the following compound 7-1 to compound 10-189.

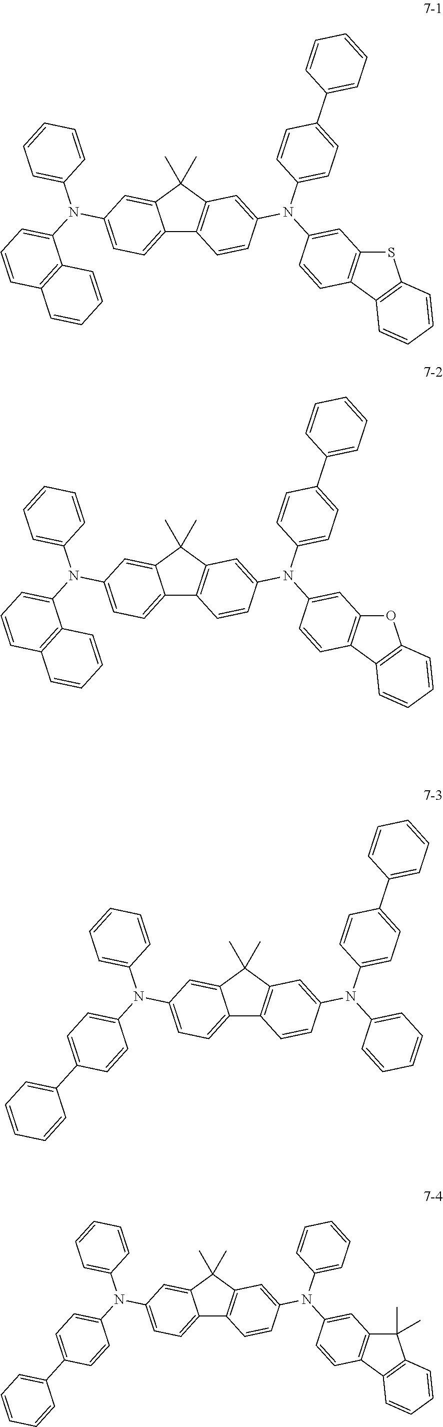

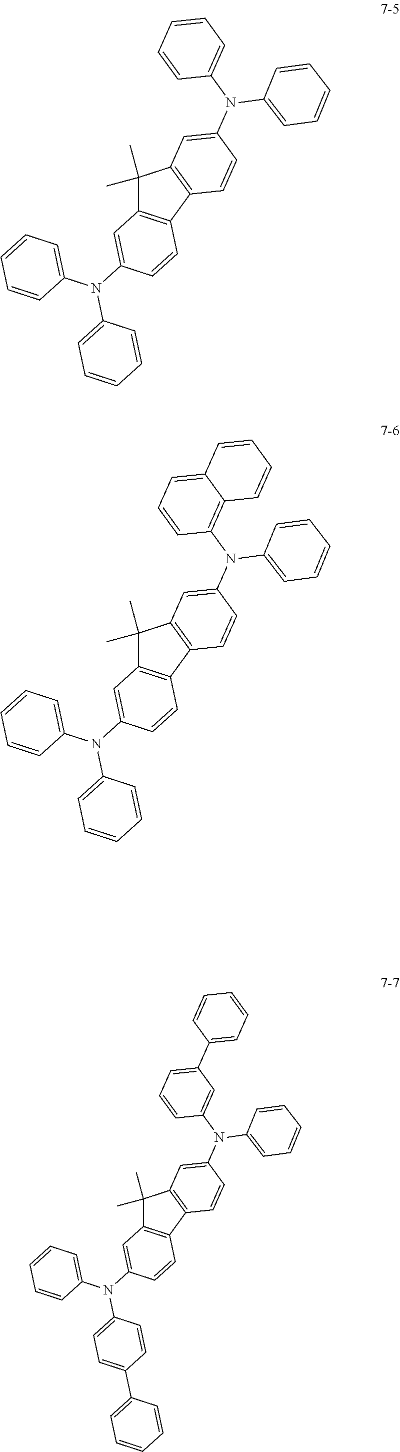

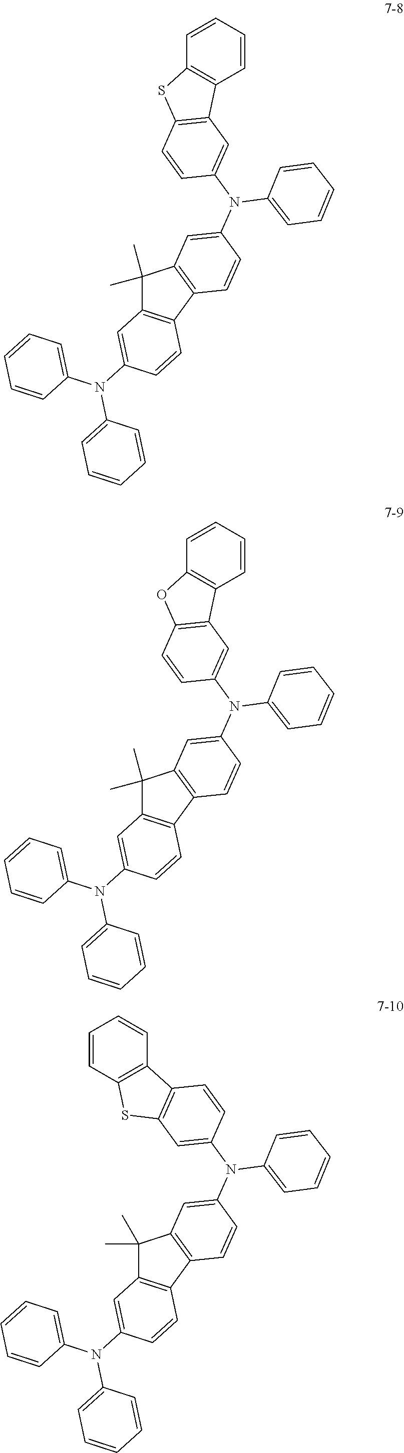

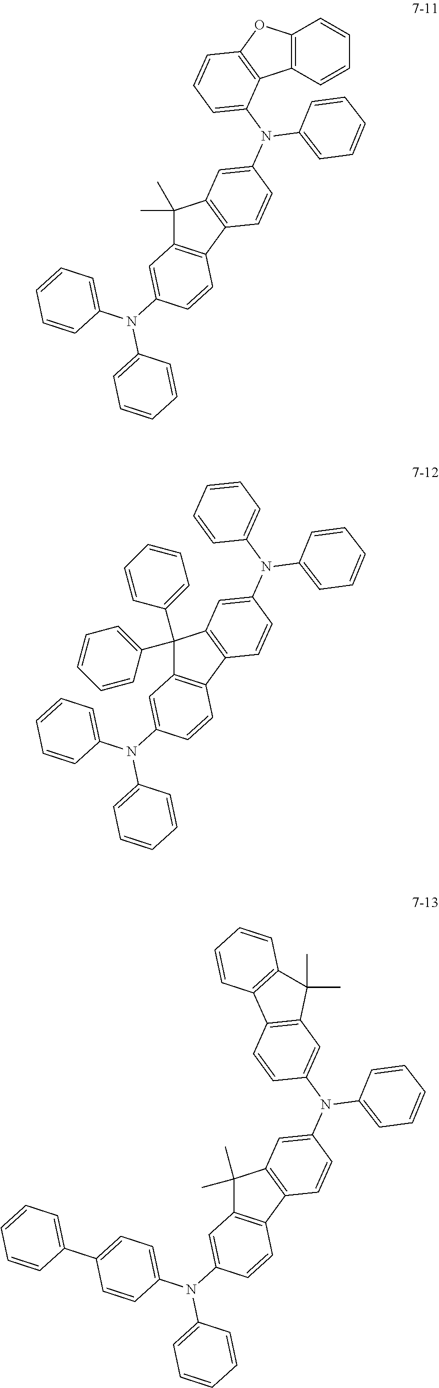

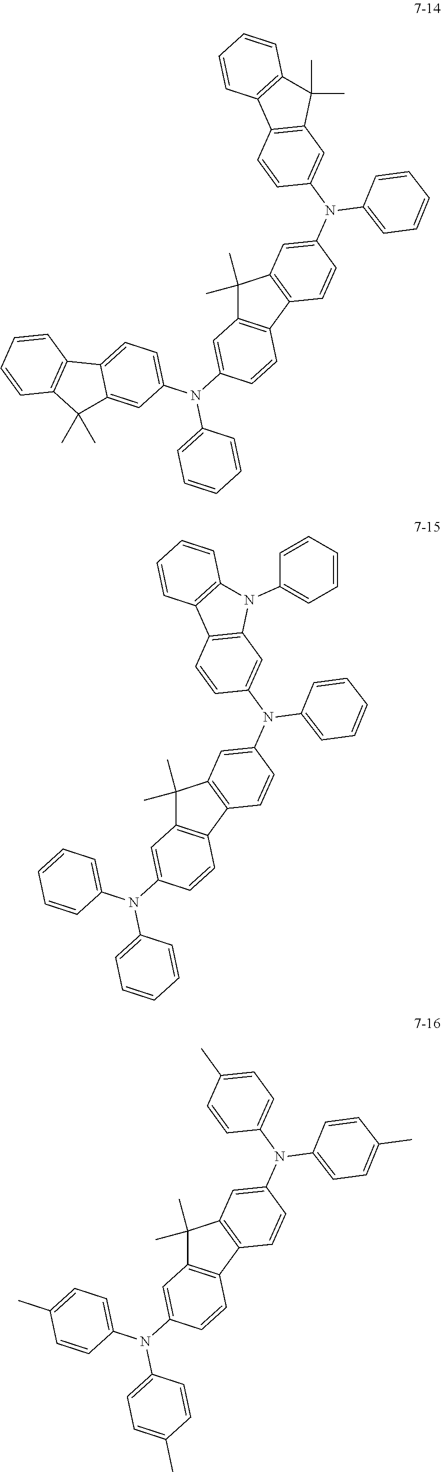

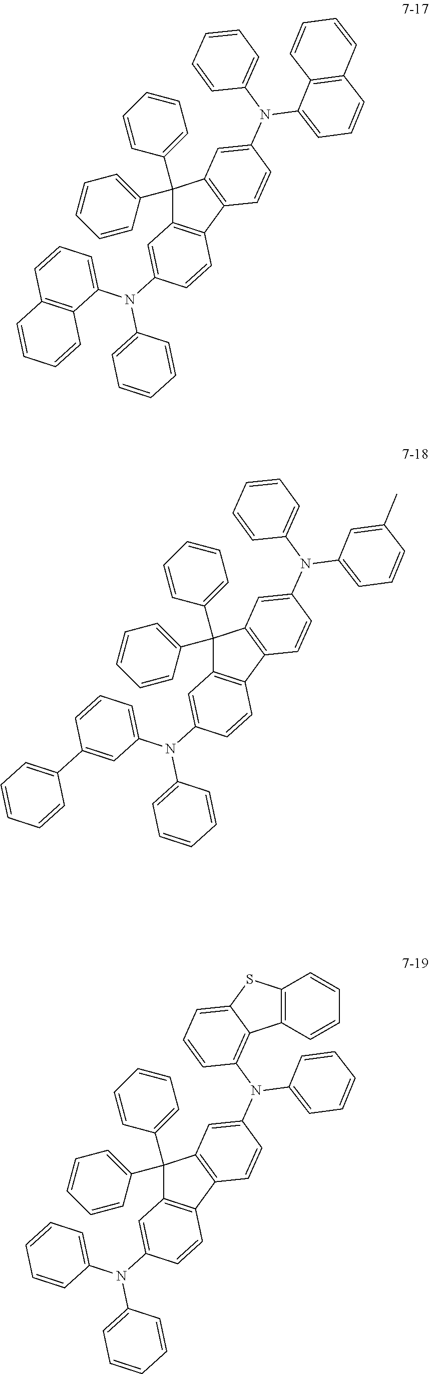

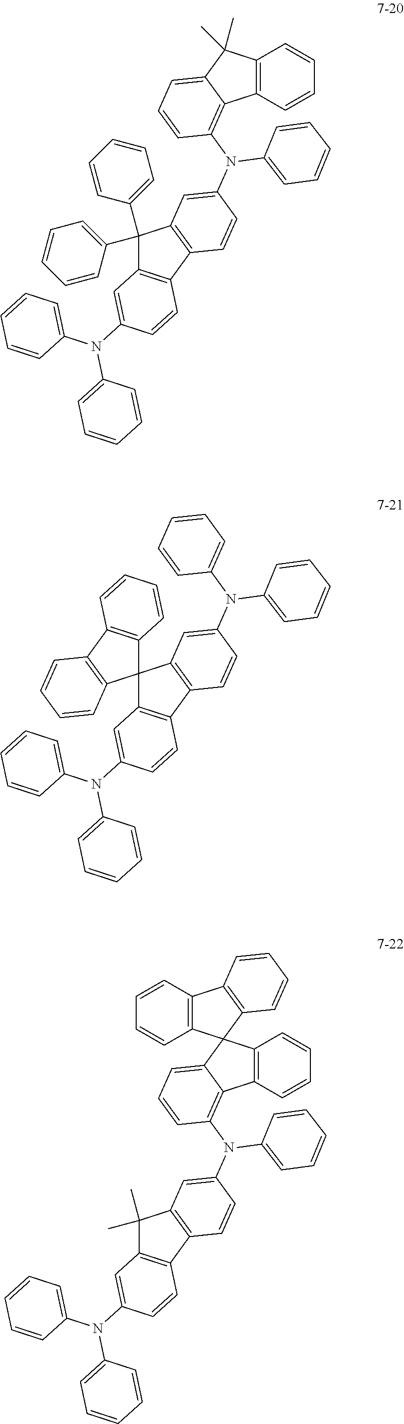

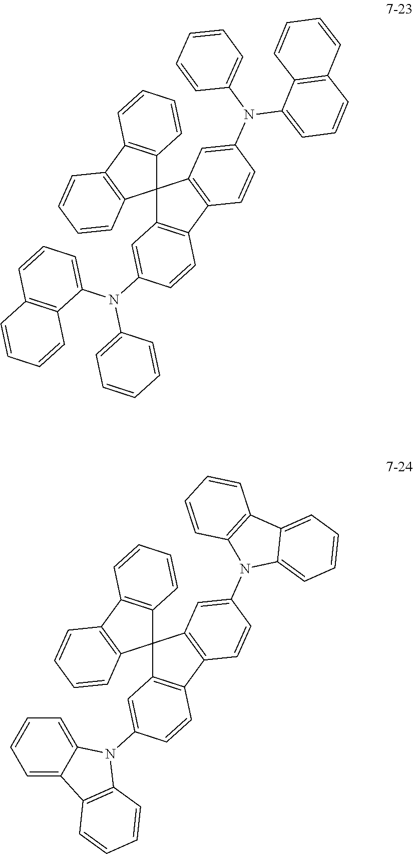

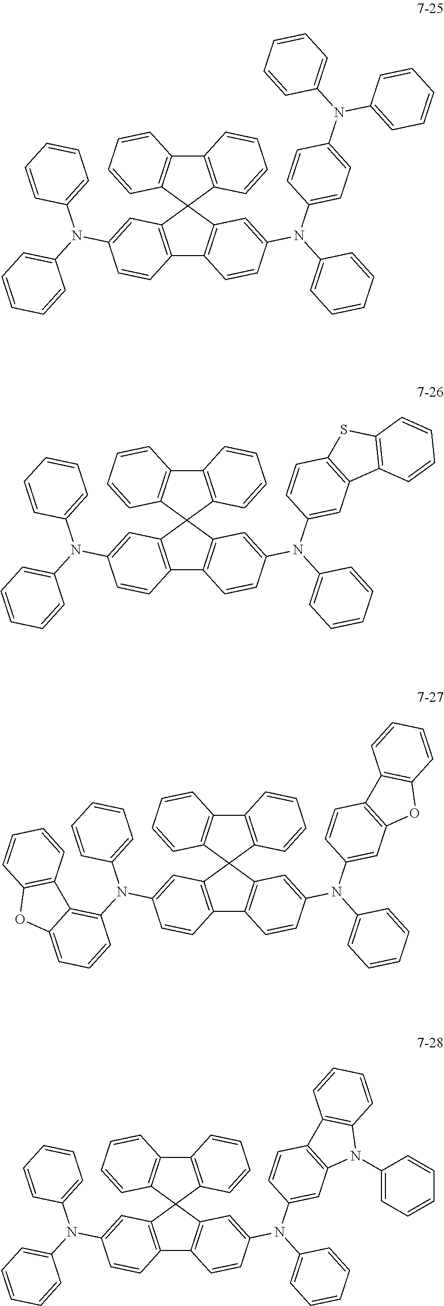

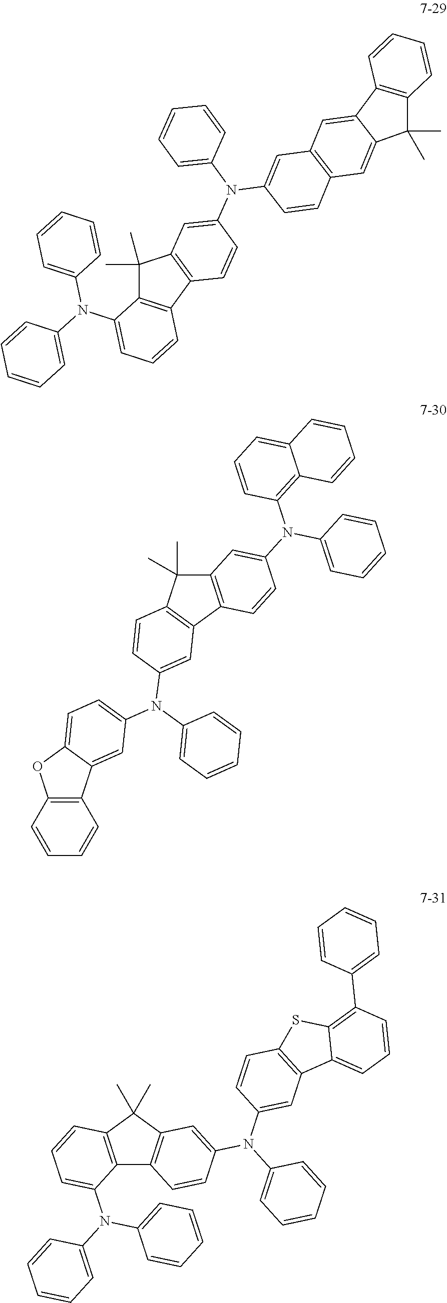

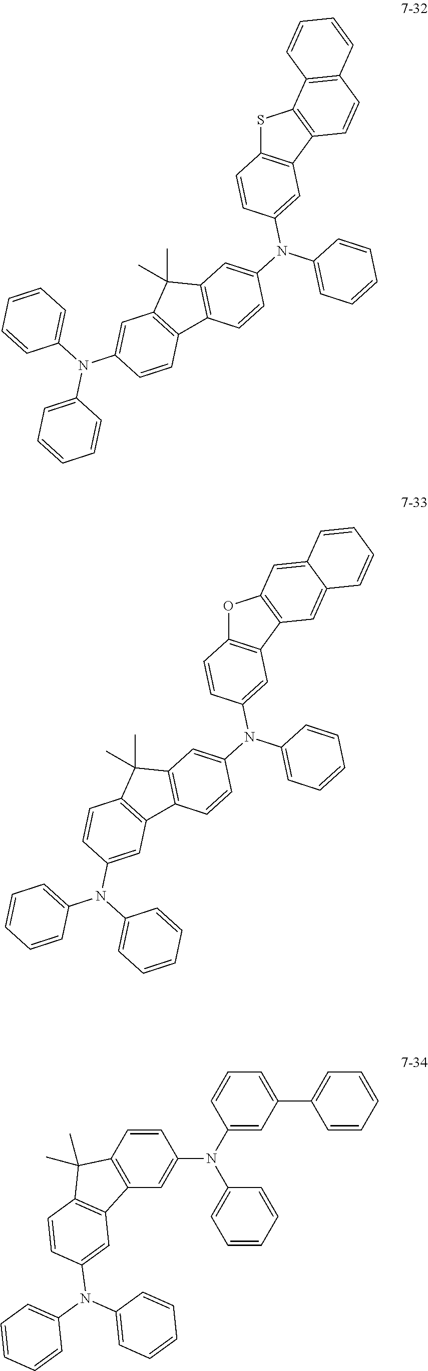

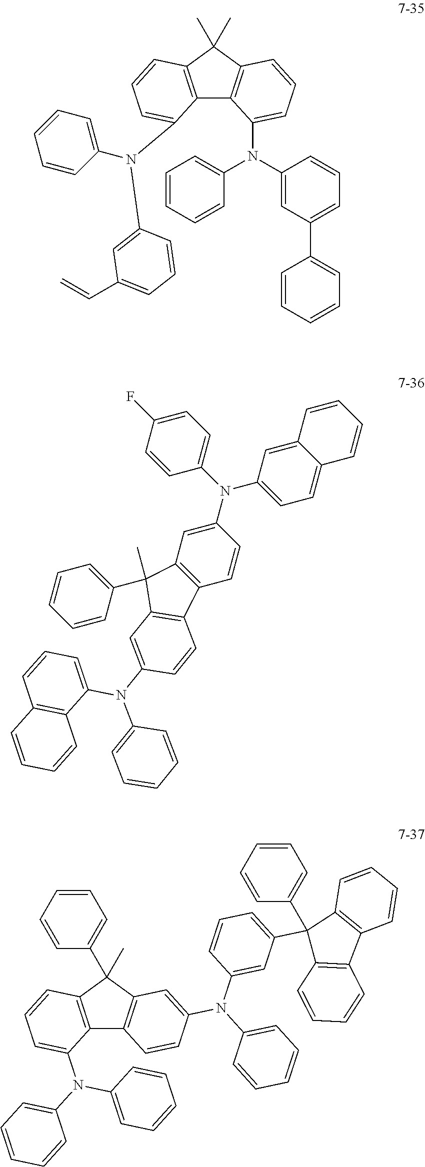

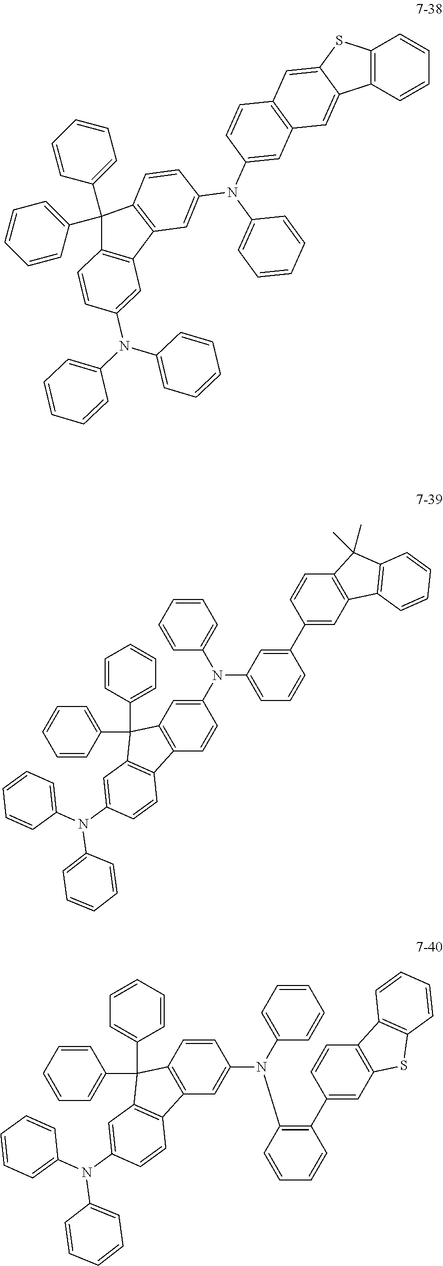

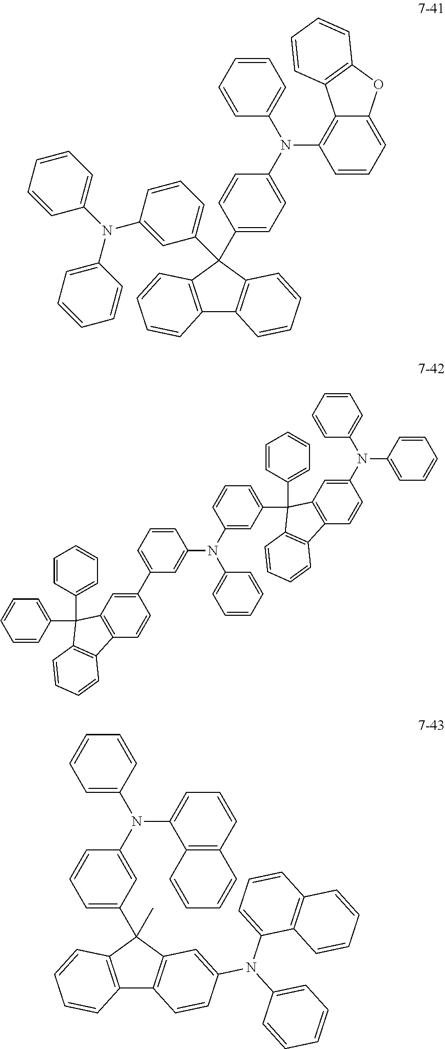

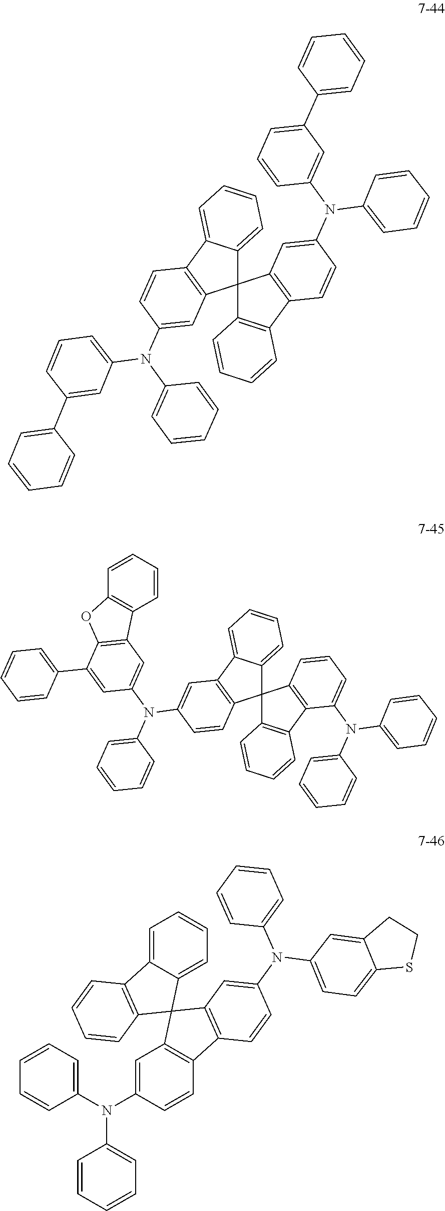

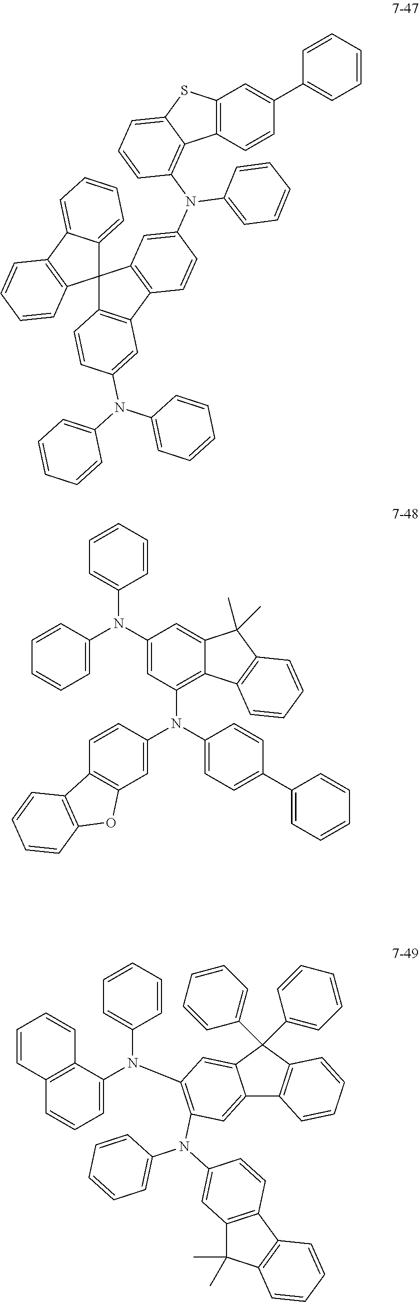

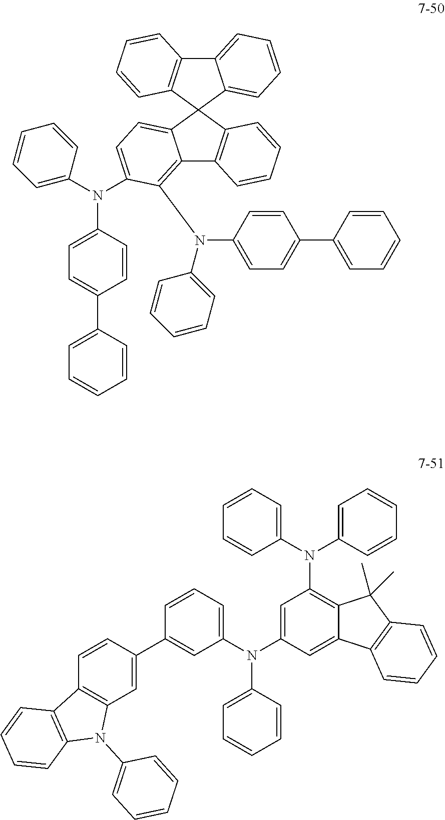

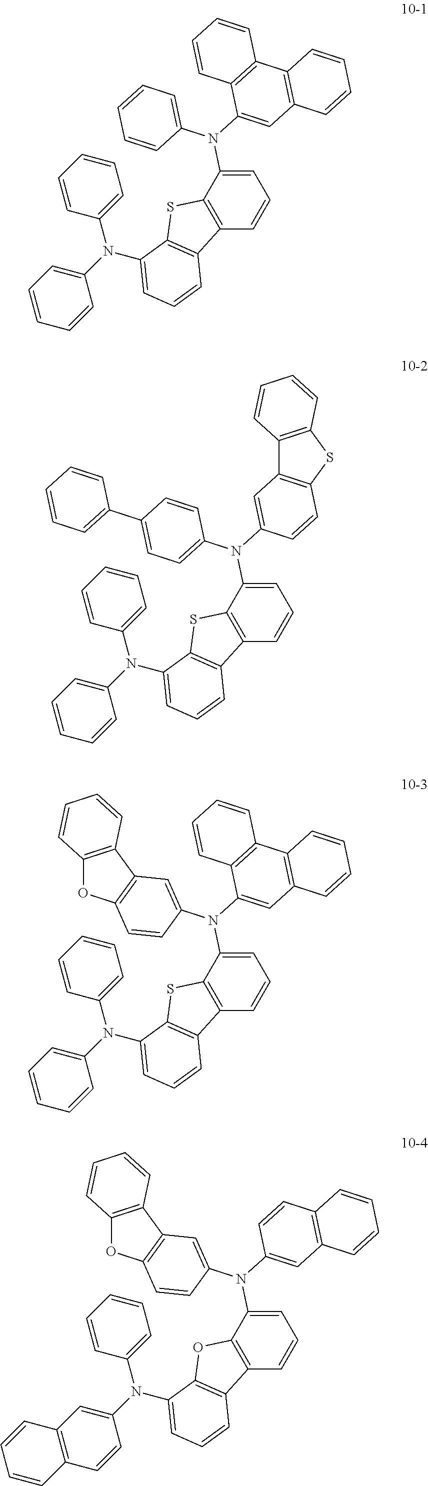

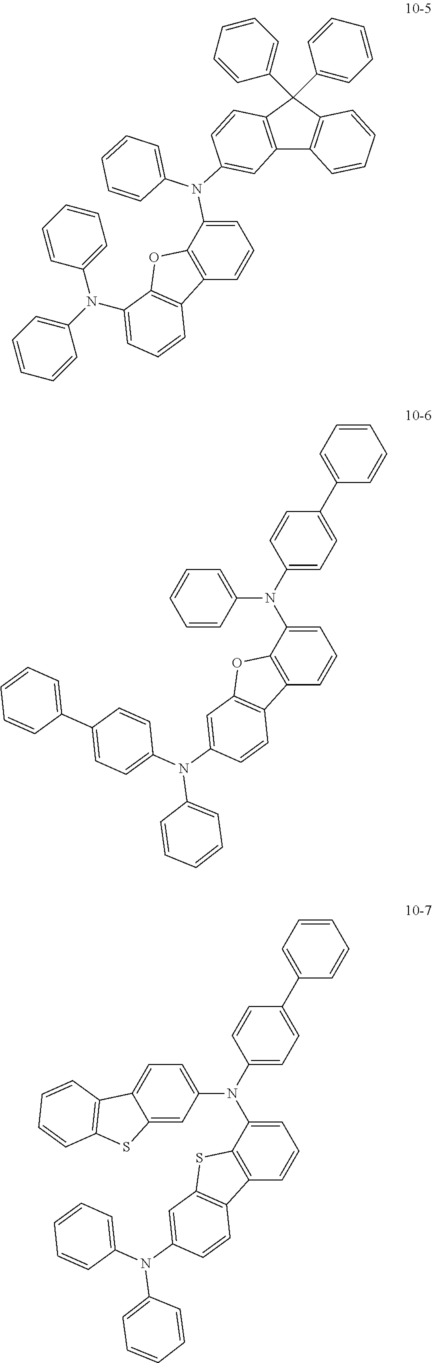

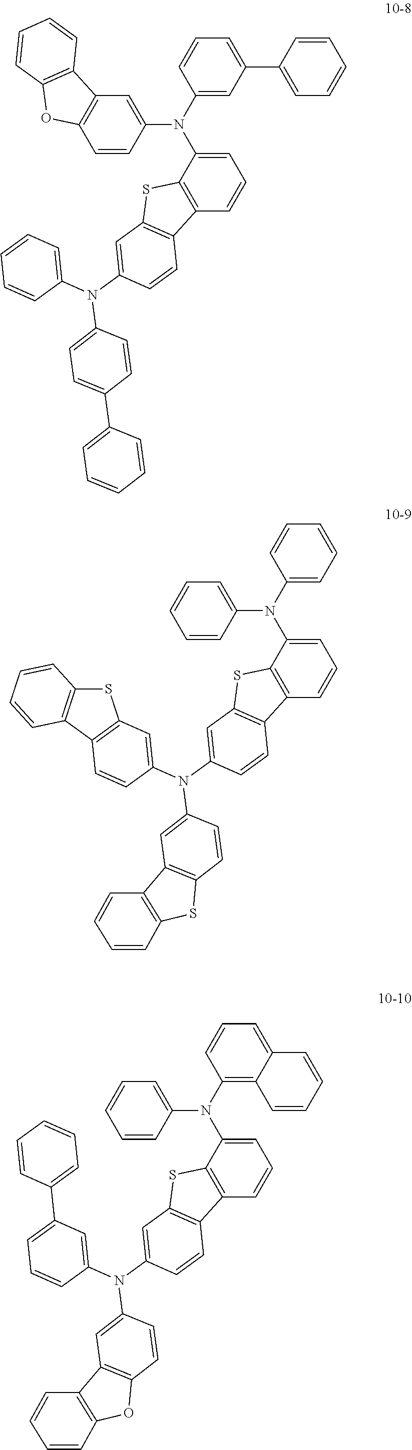

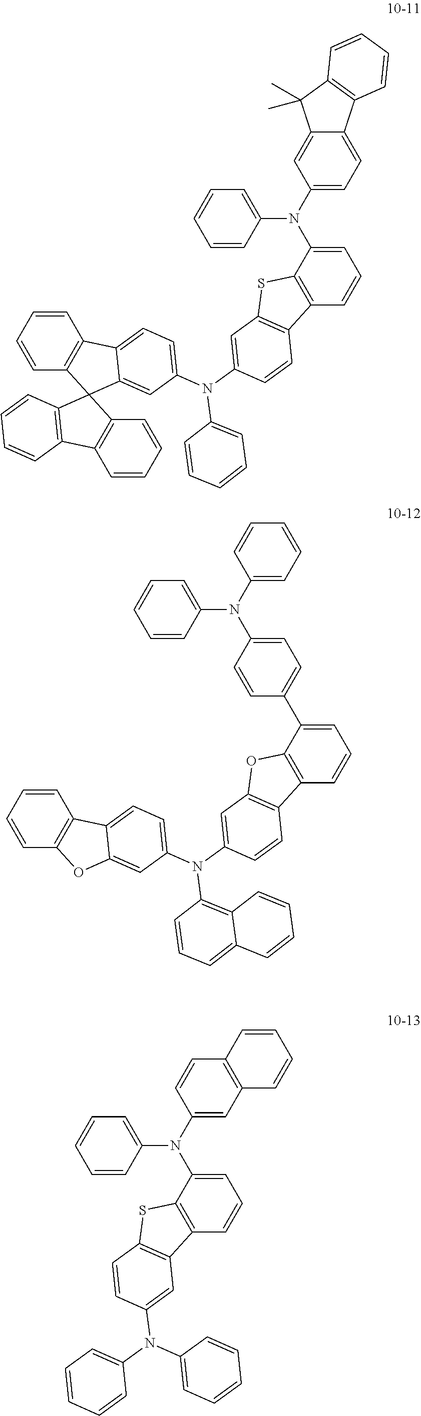

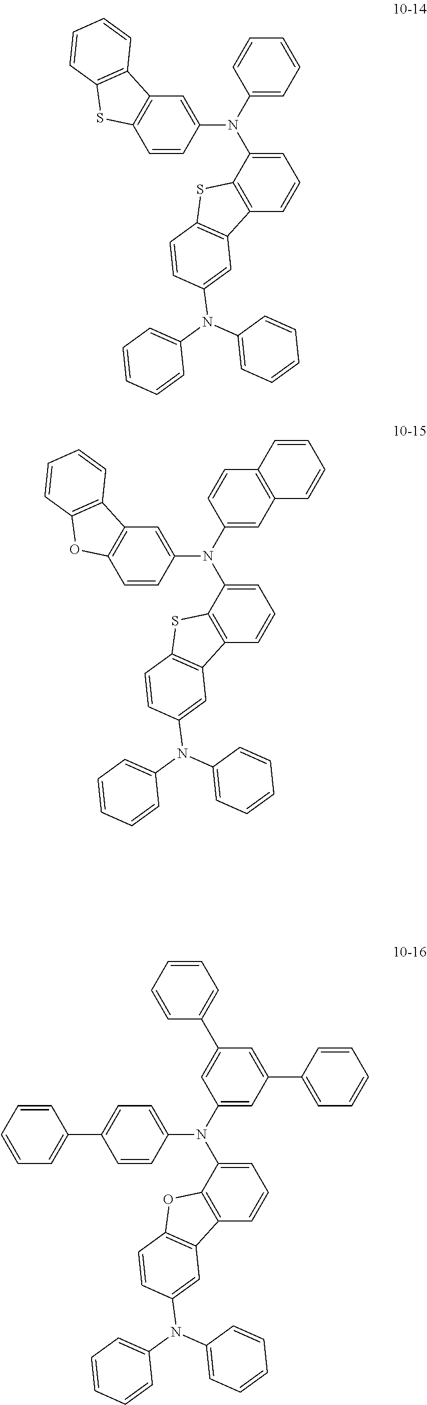

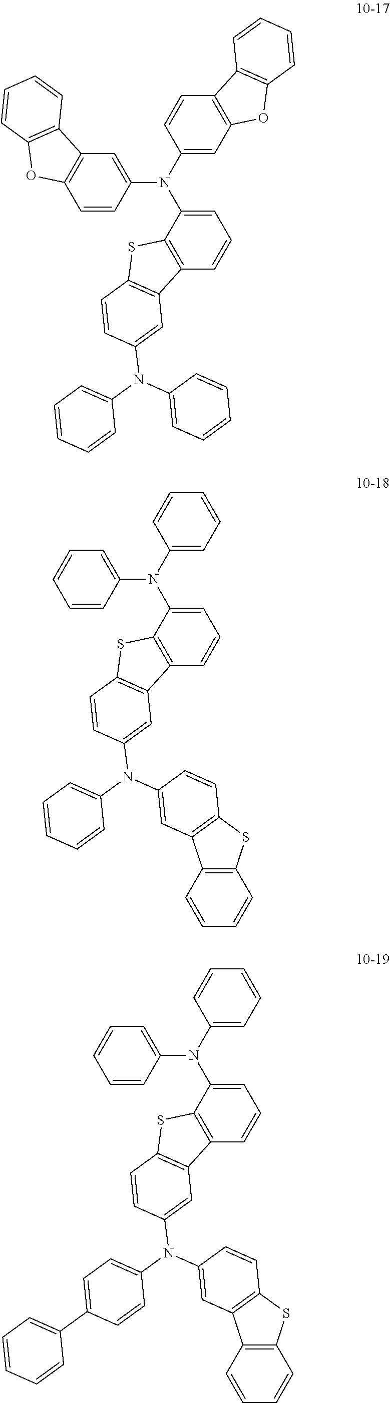

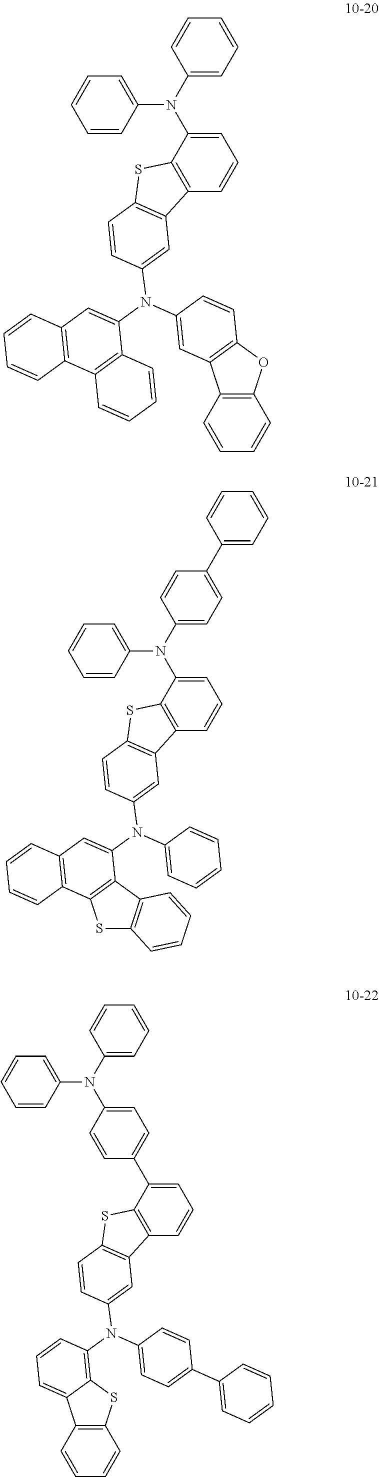

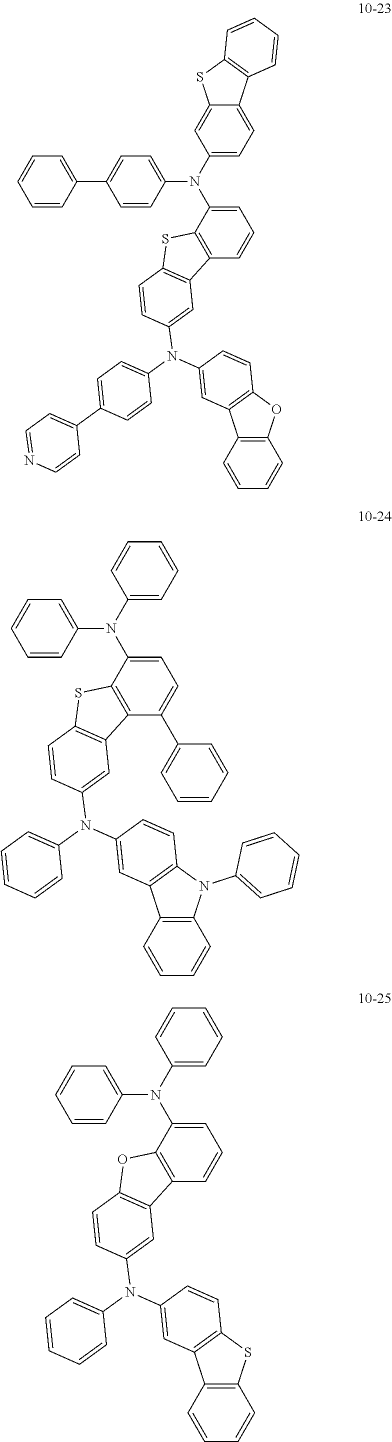

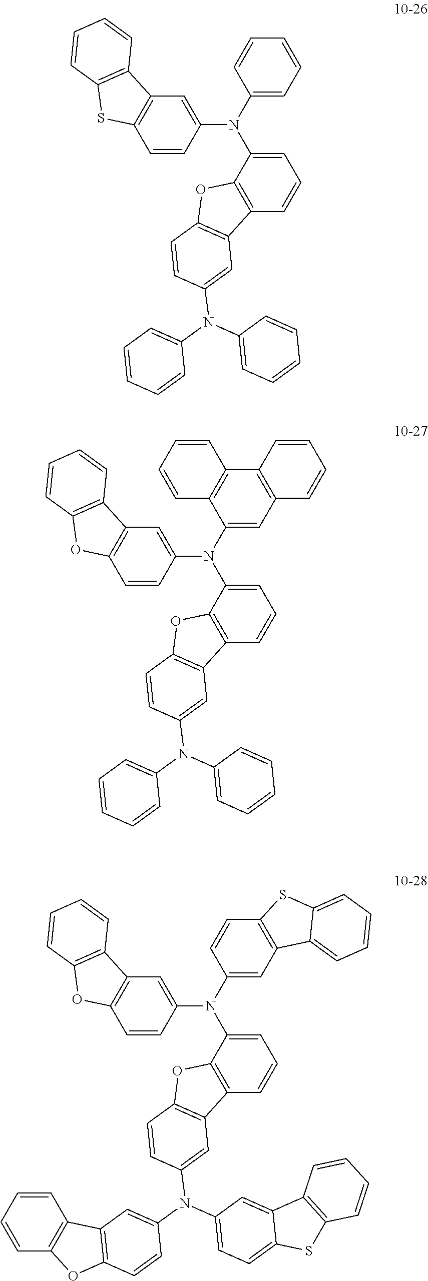

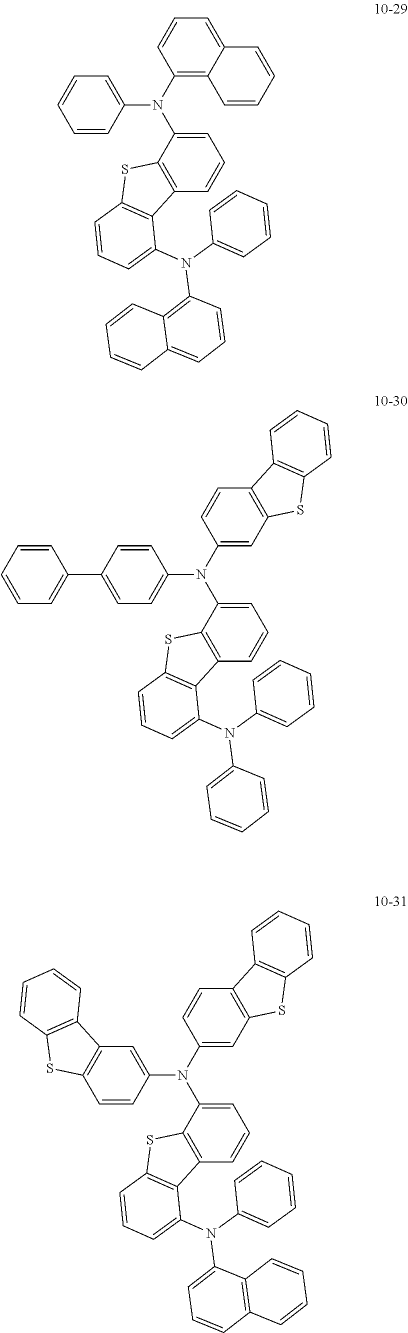

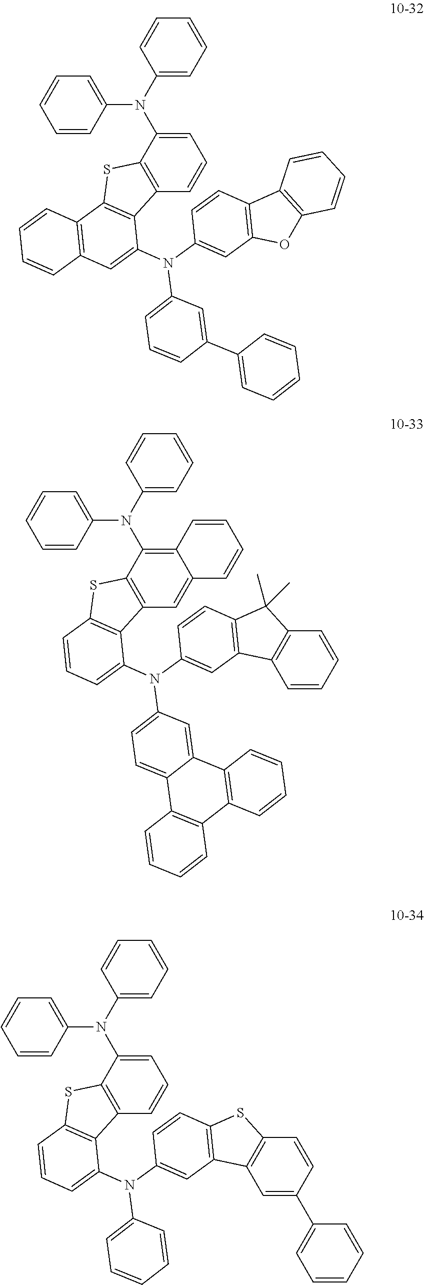

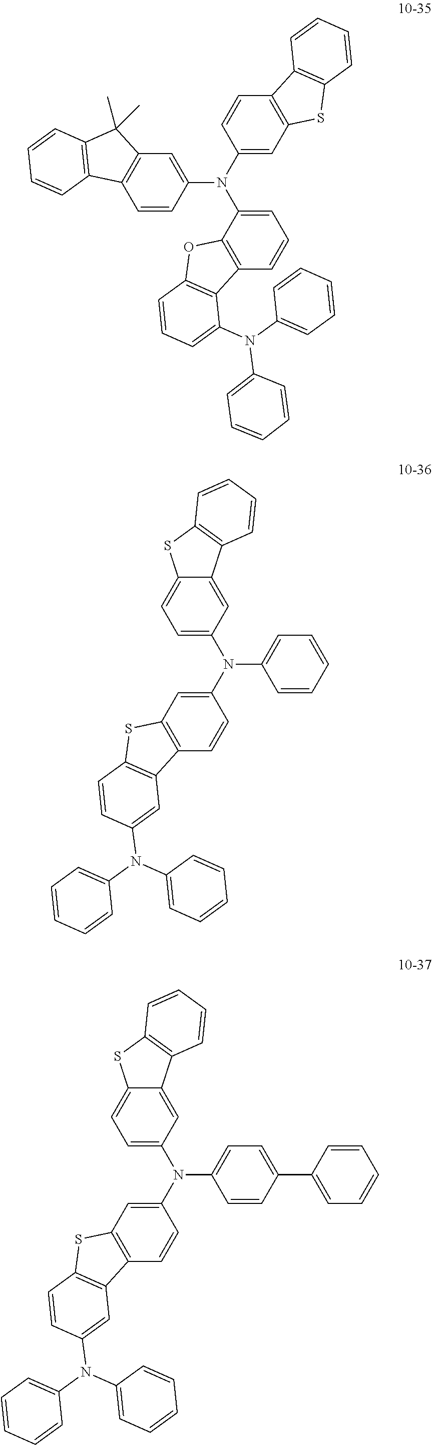

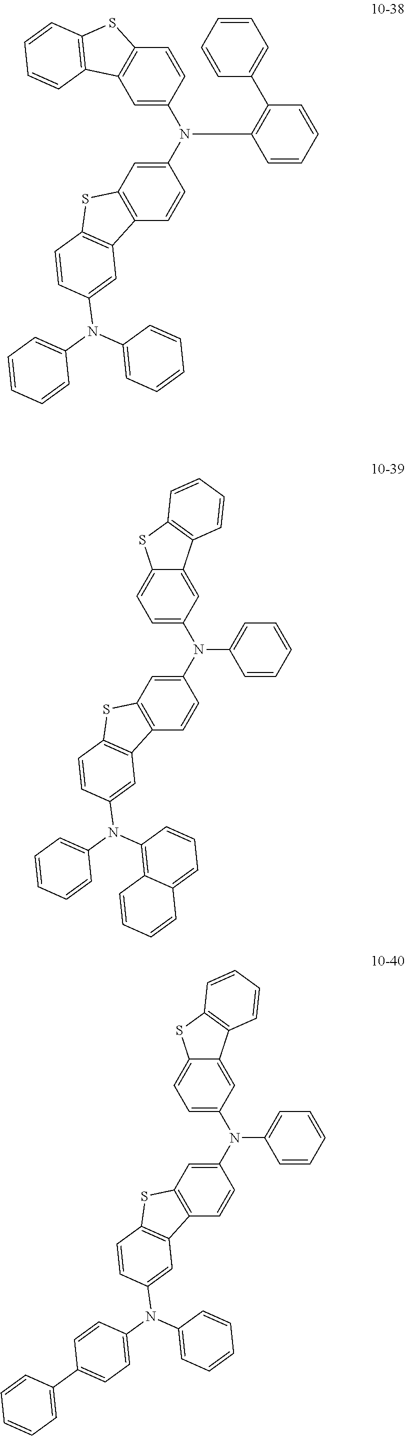

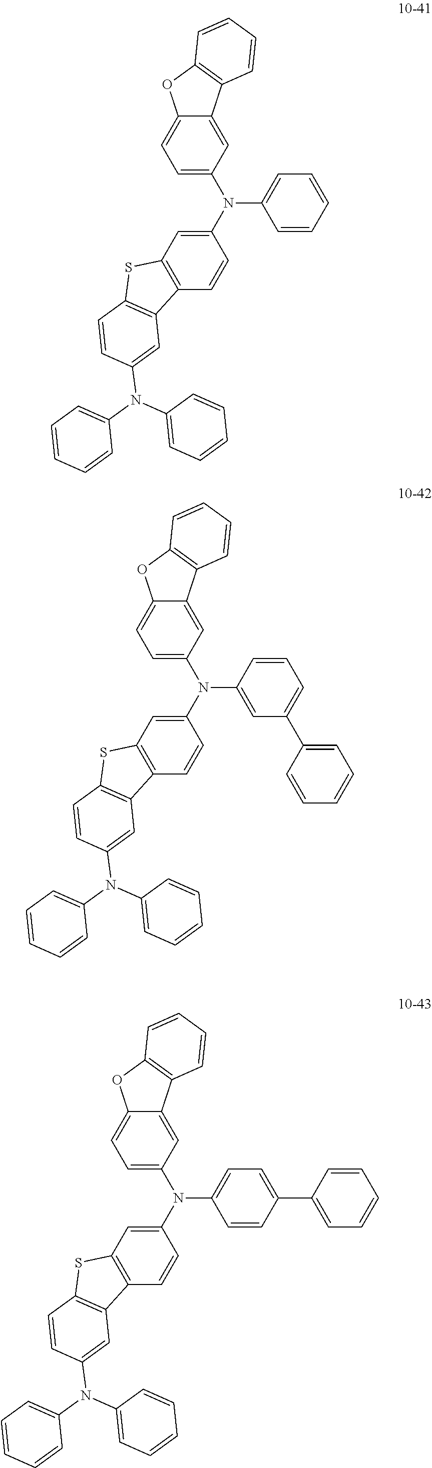

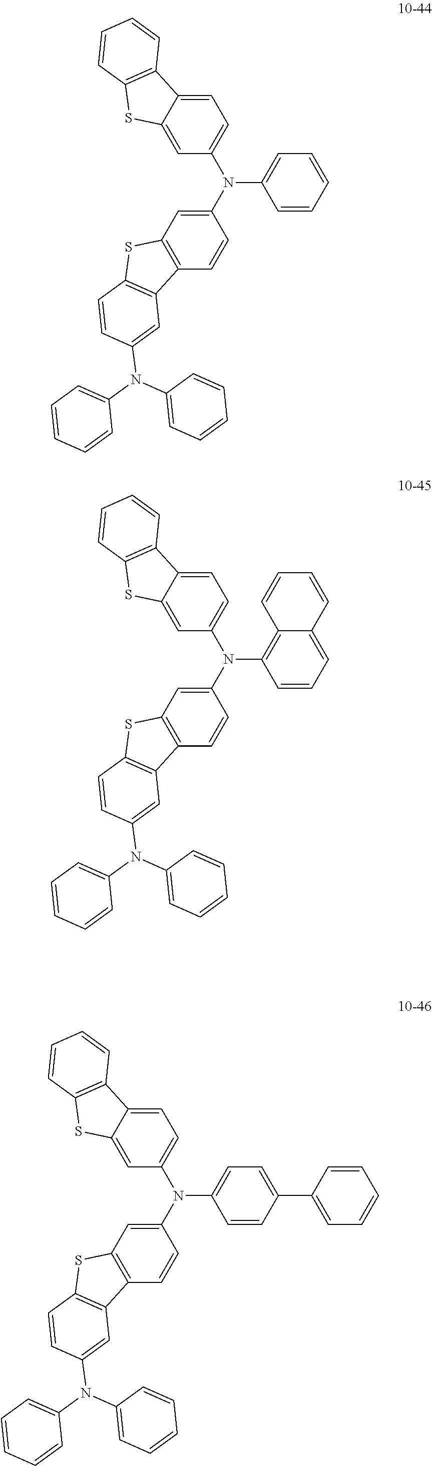

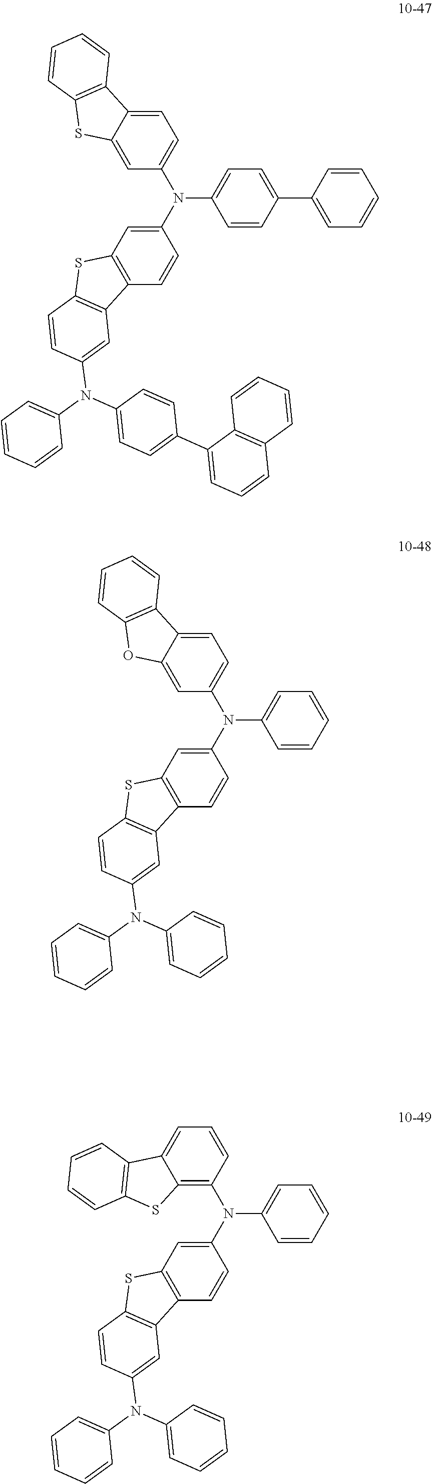

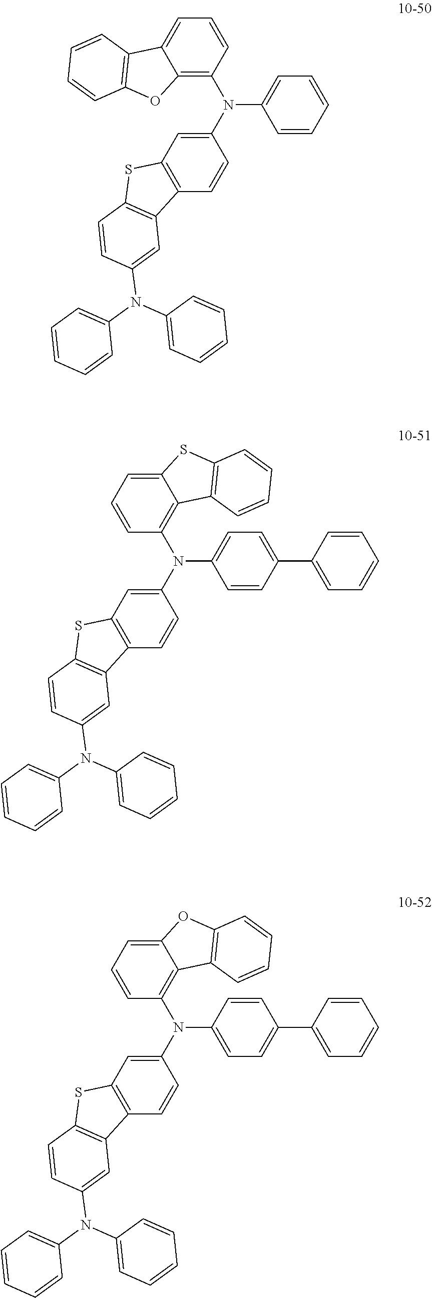

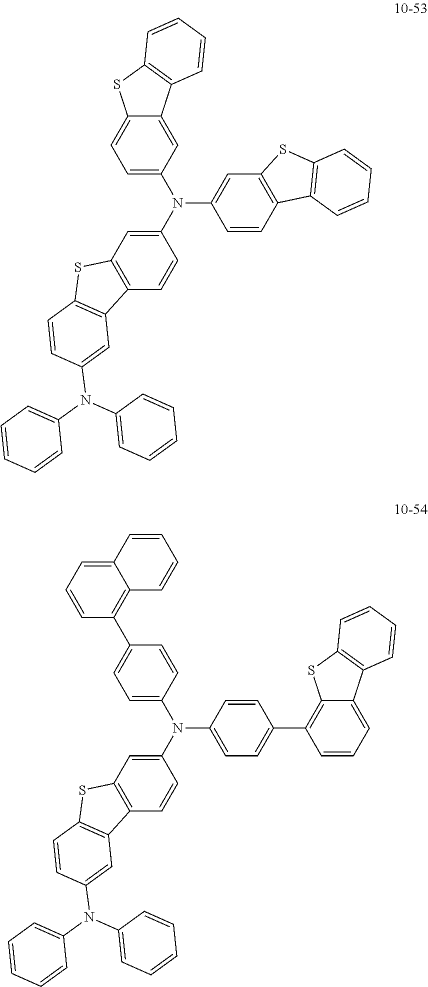

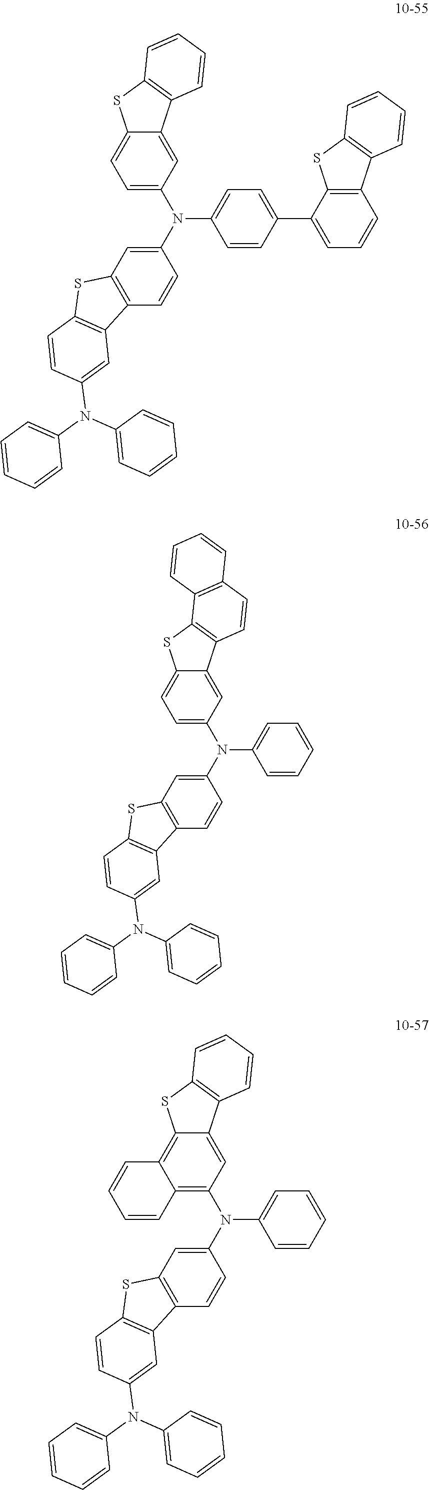

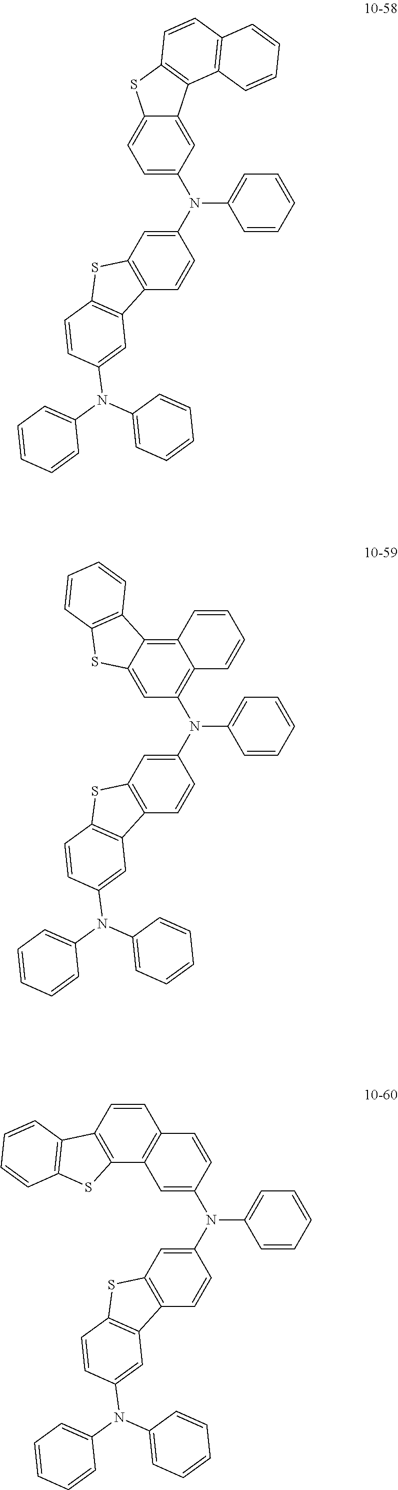

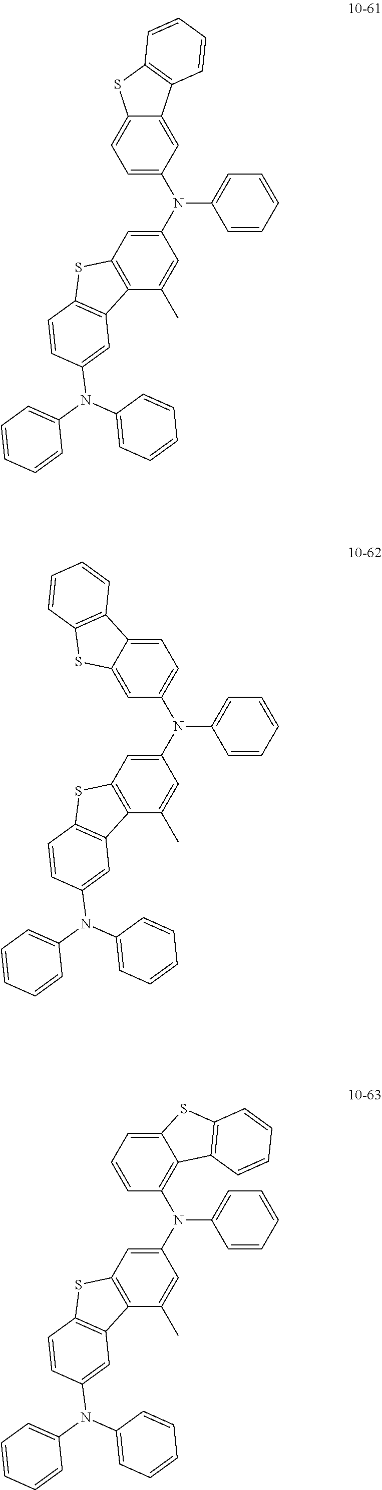

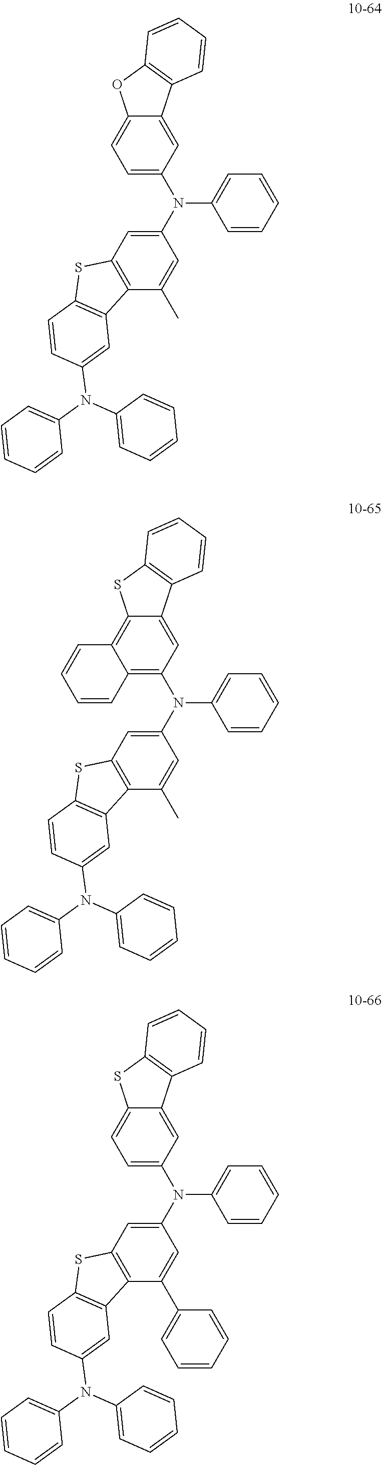

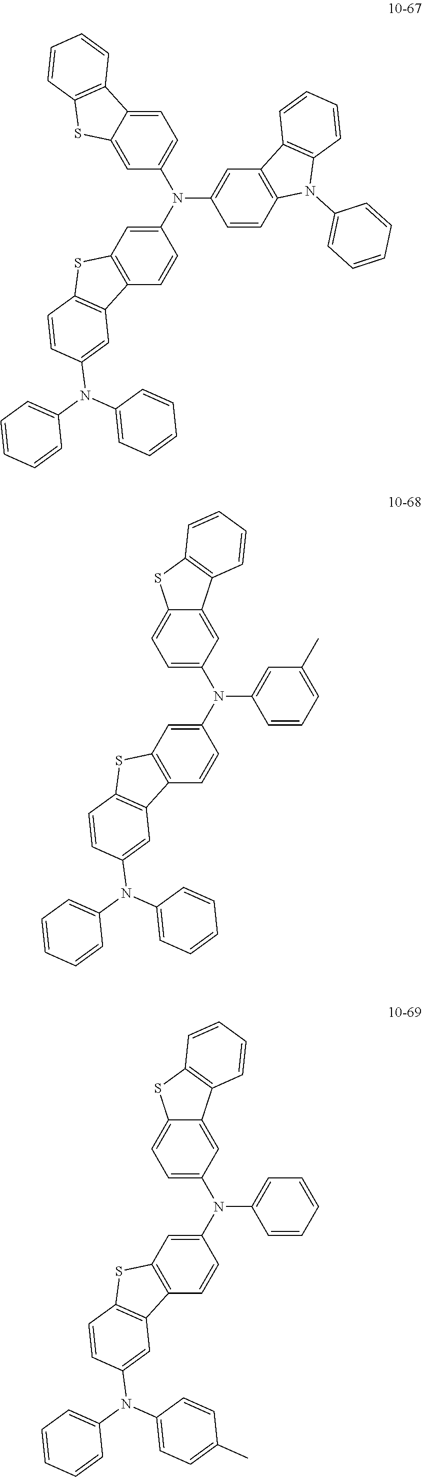

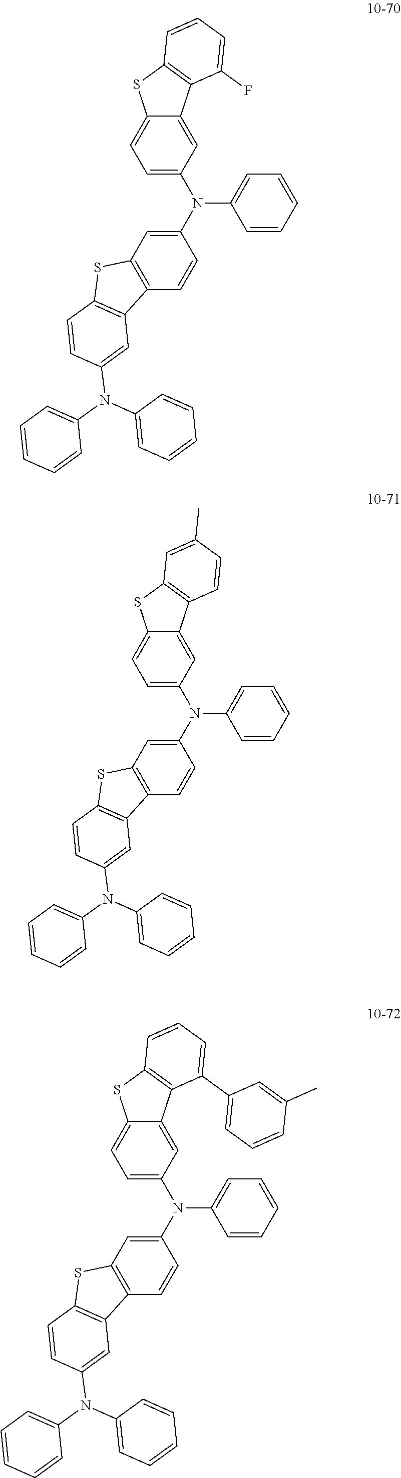

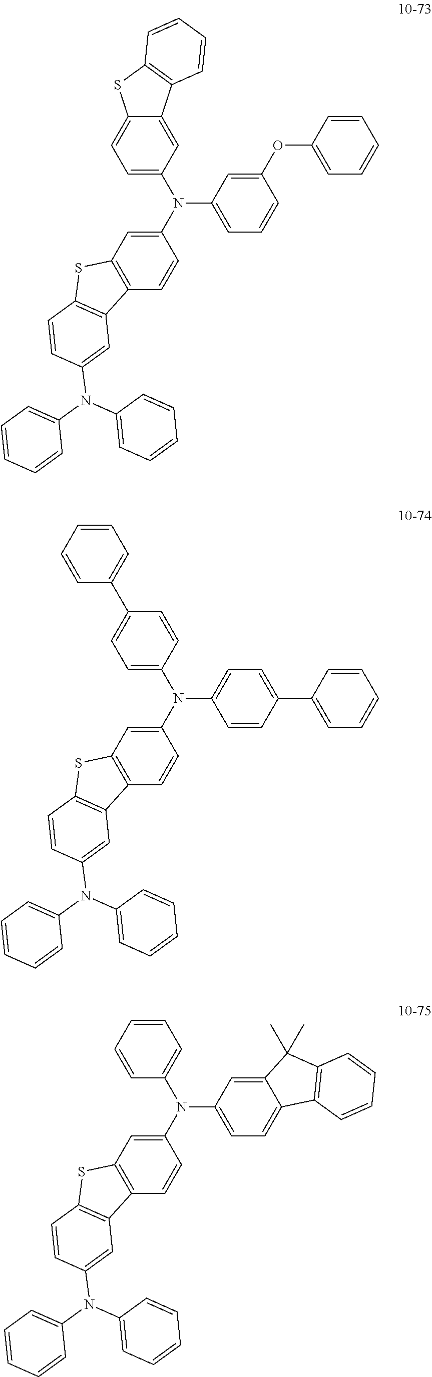

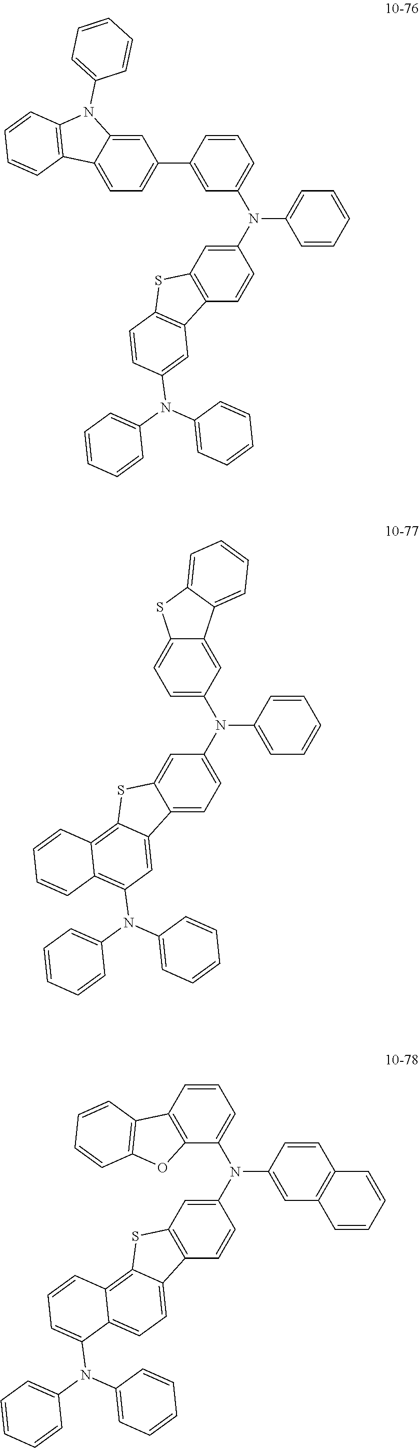

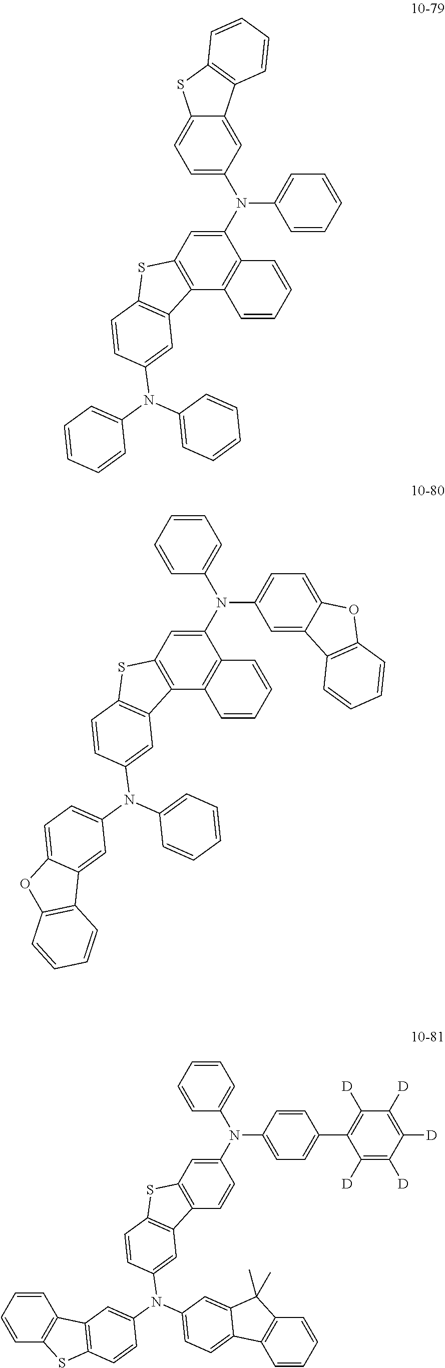

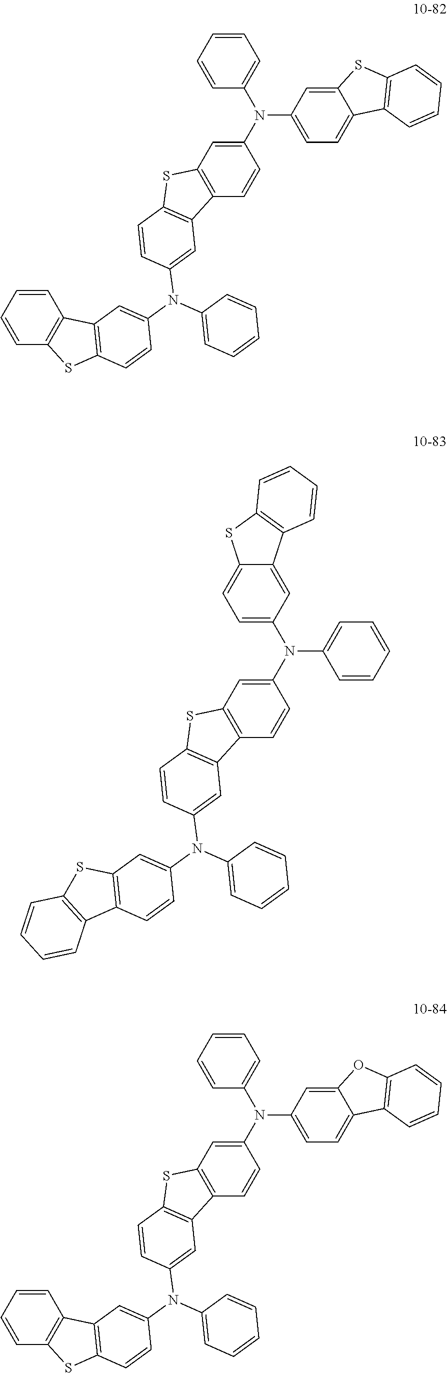

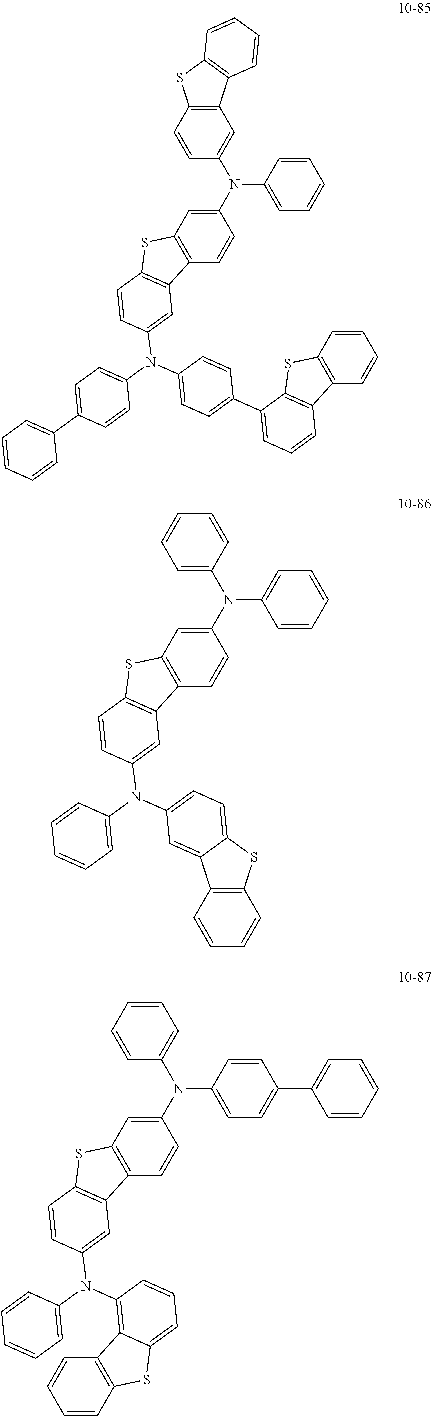

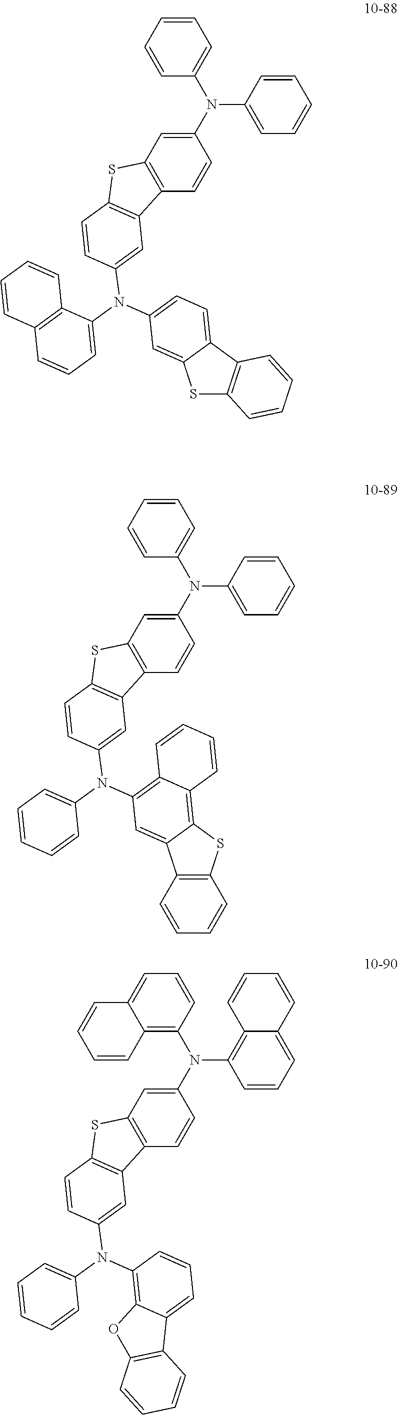

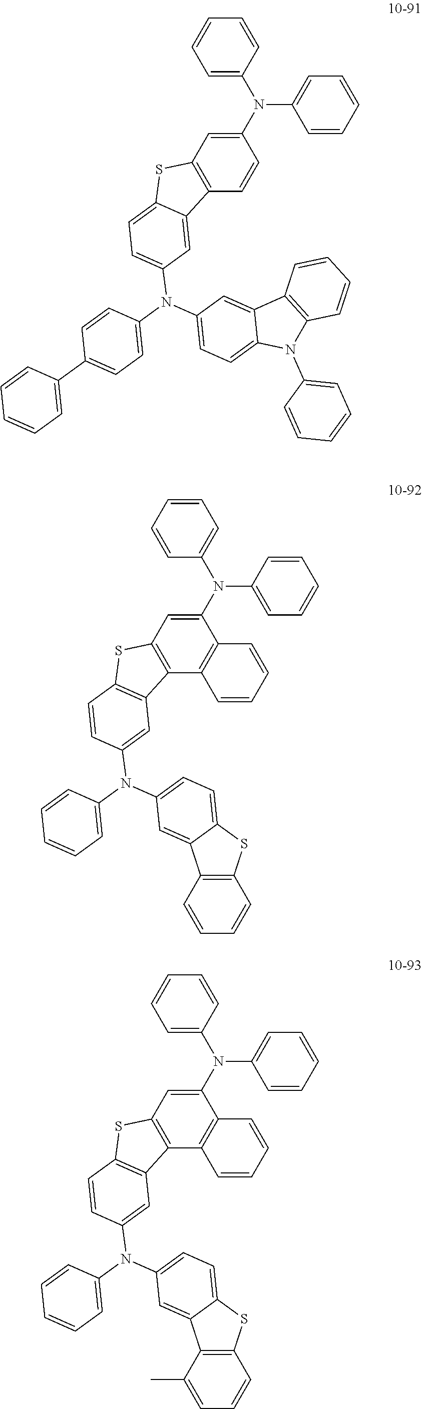

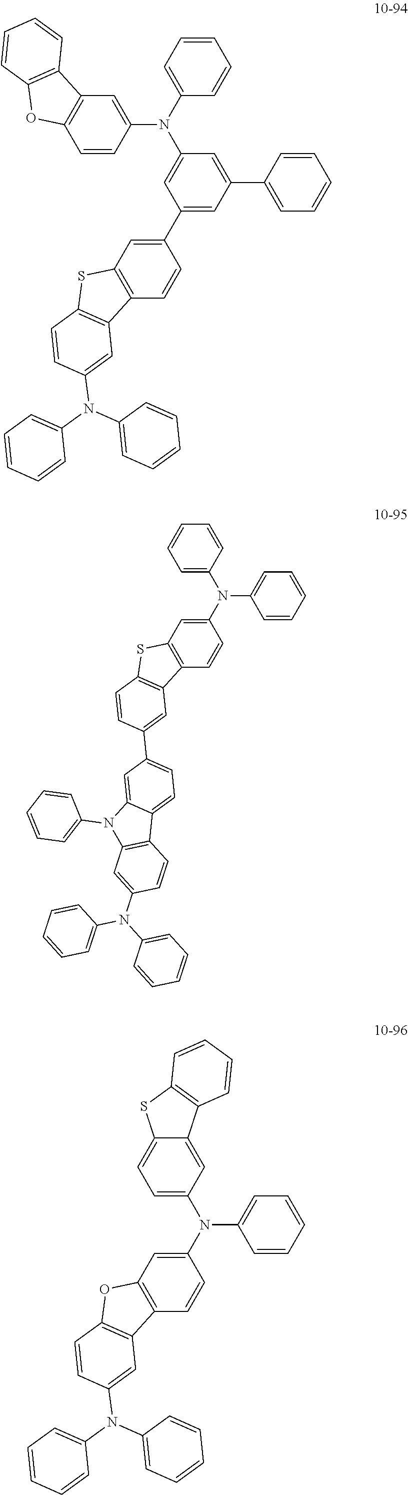

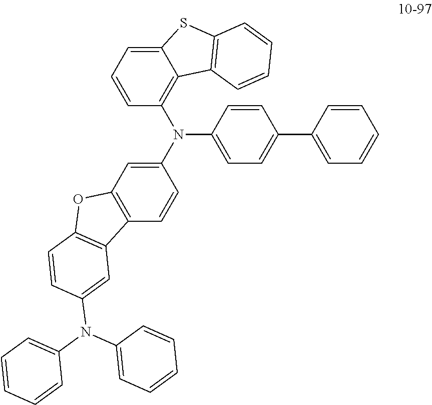

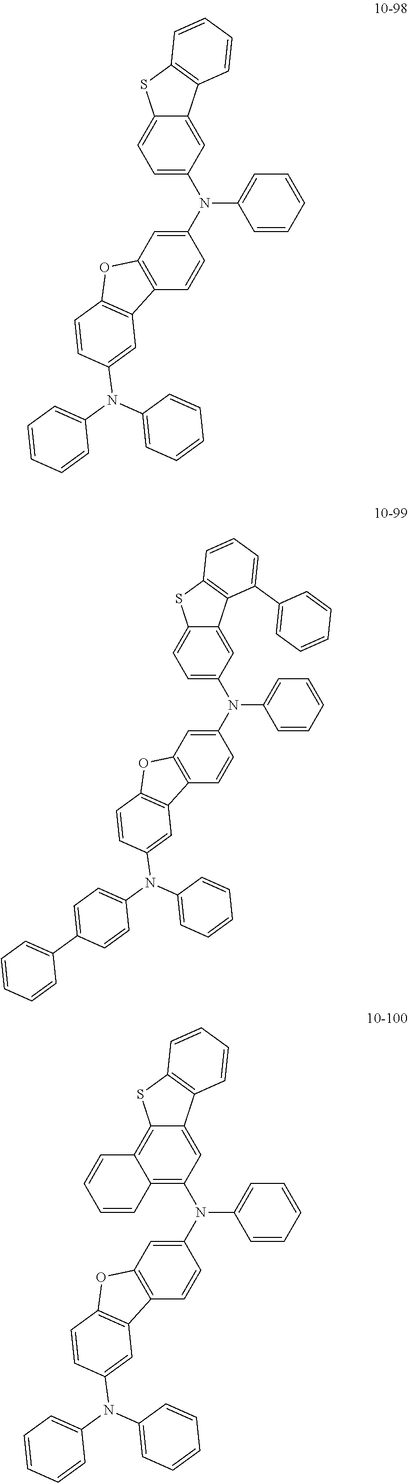

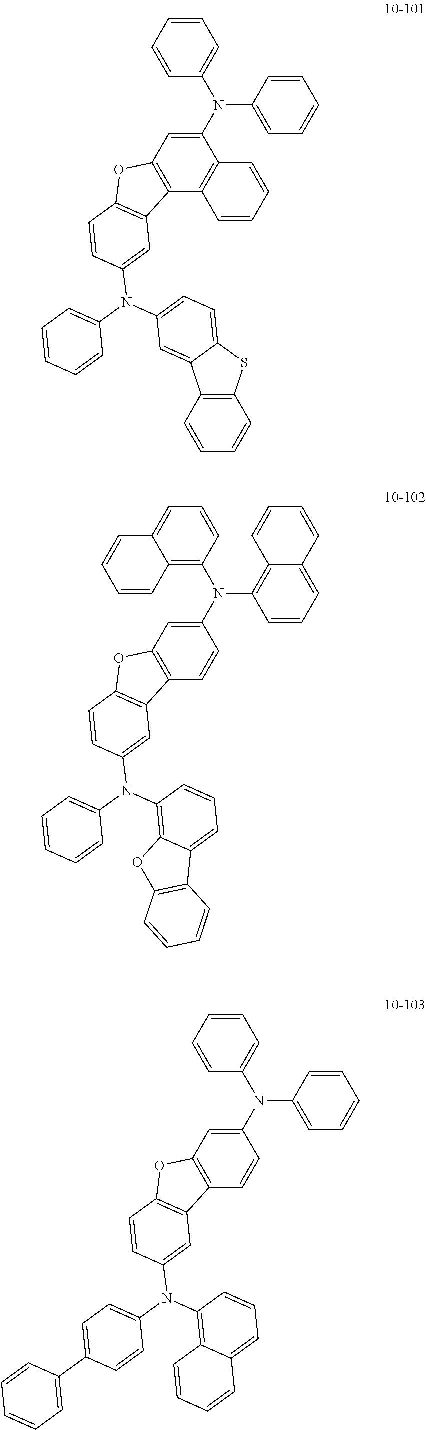

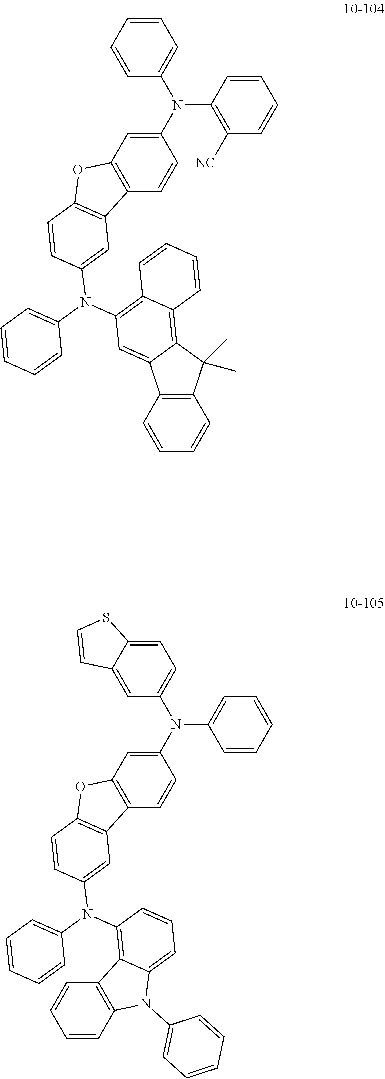

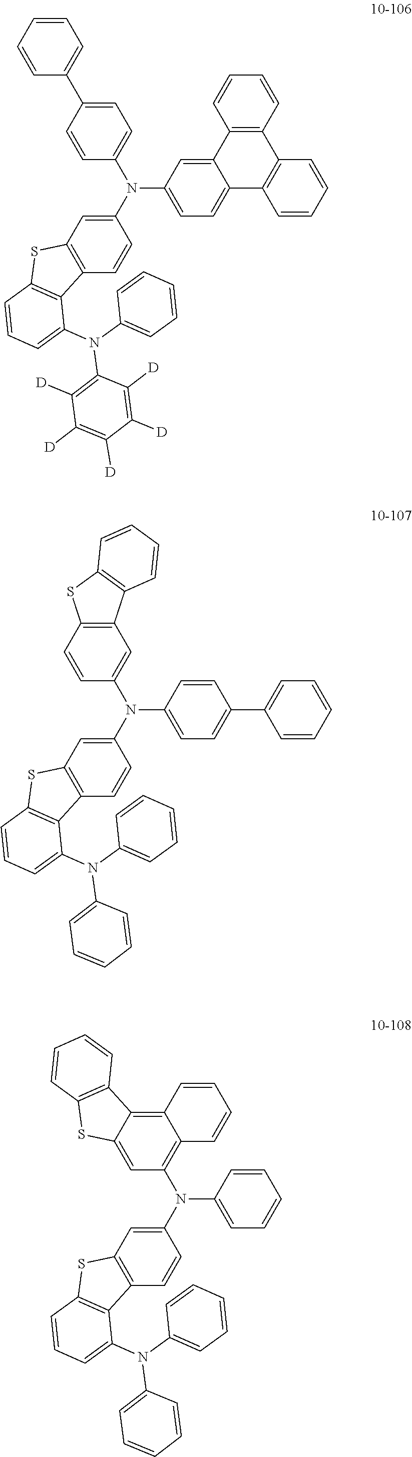

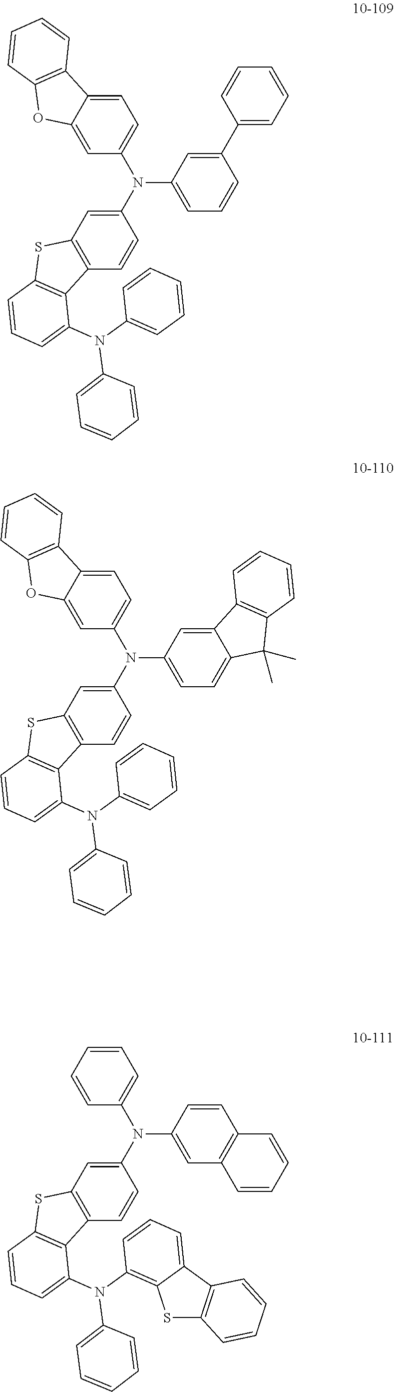

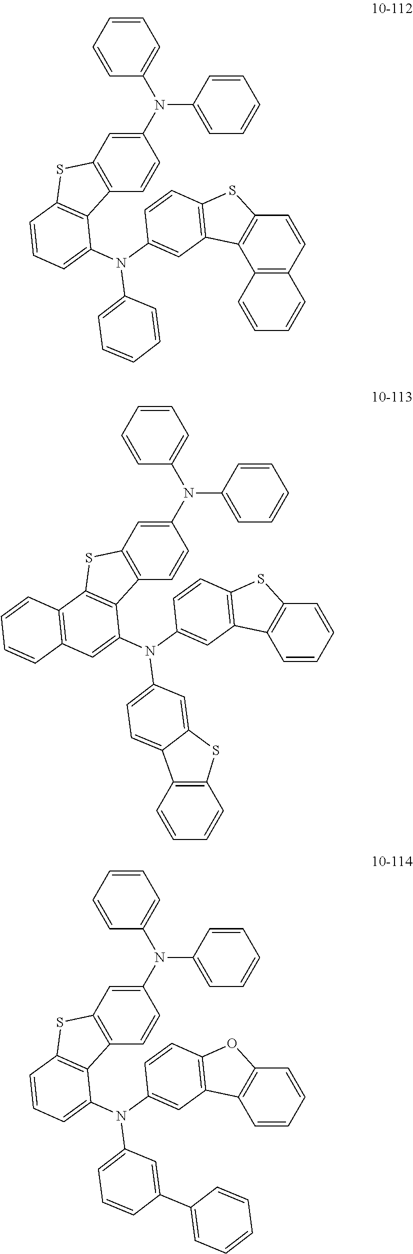

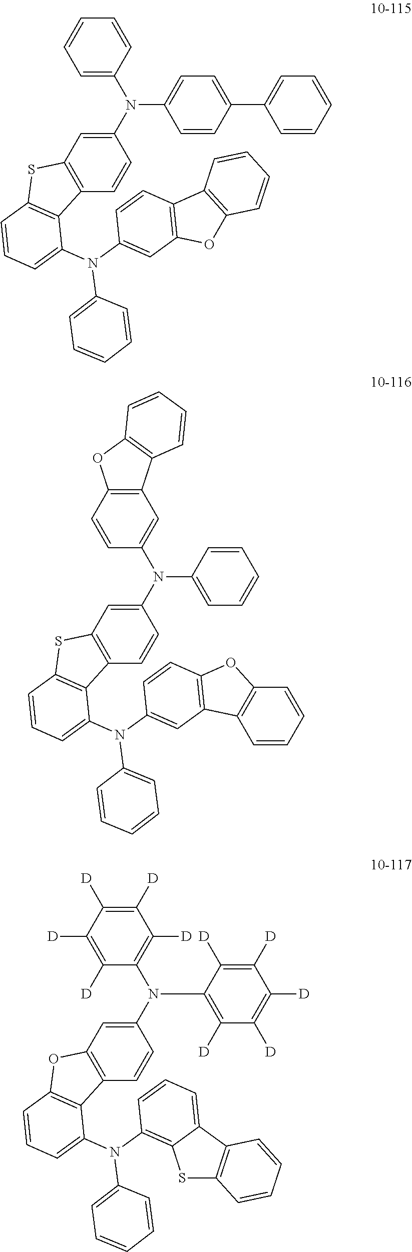

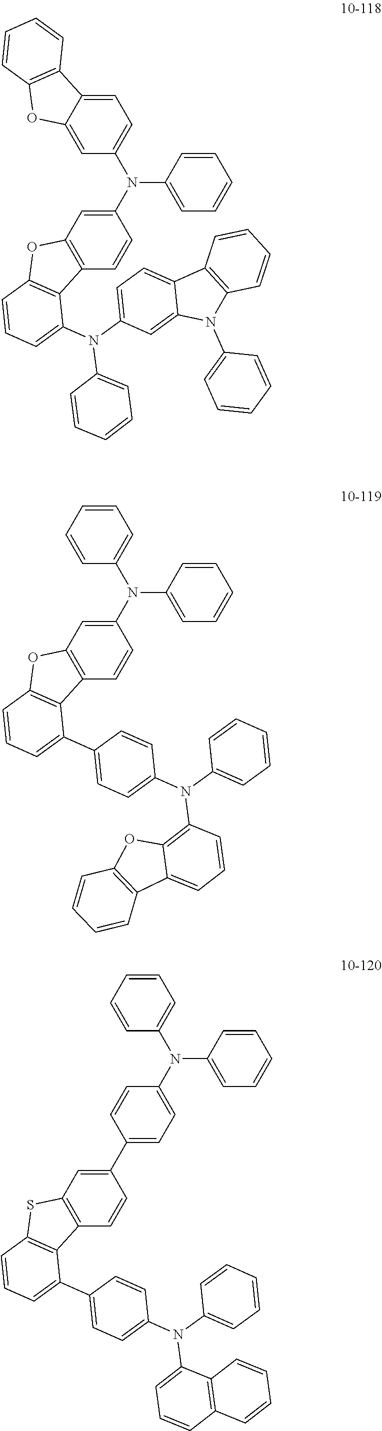

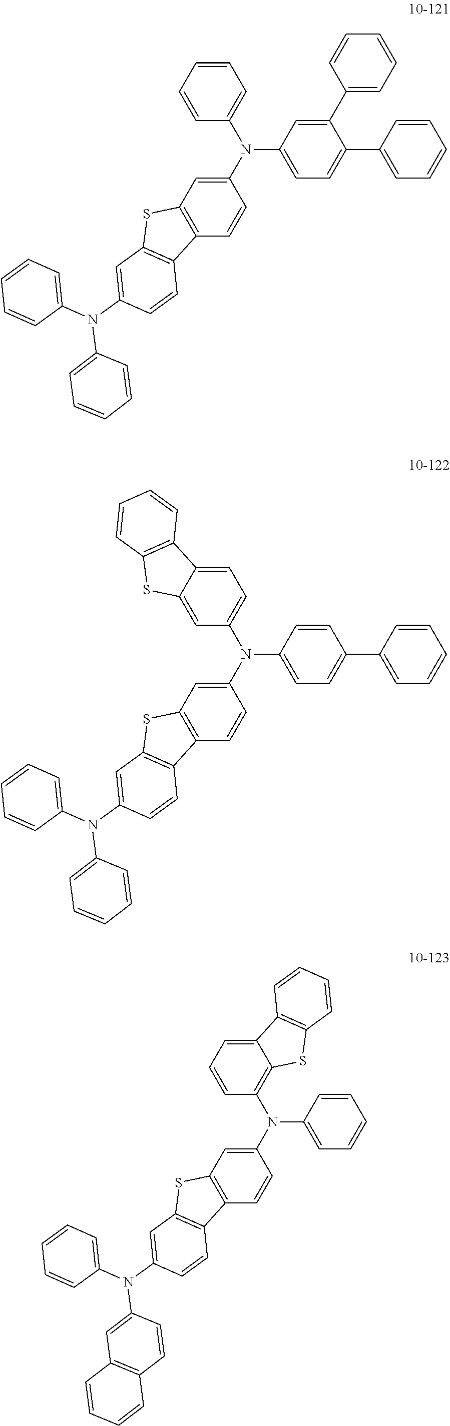

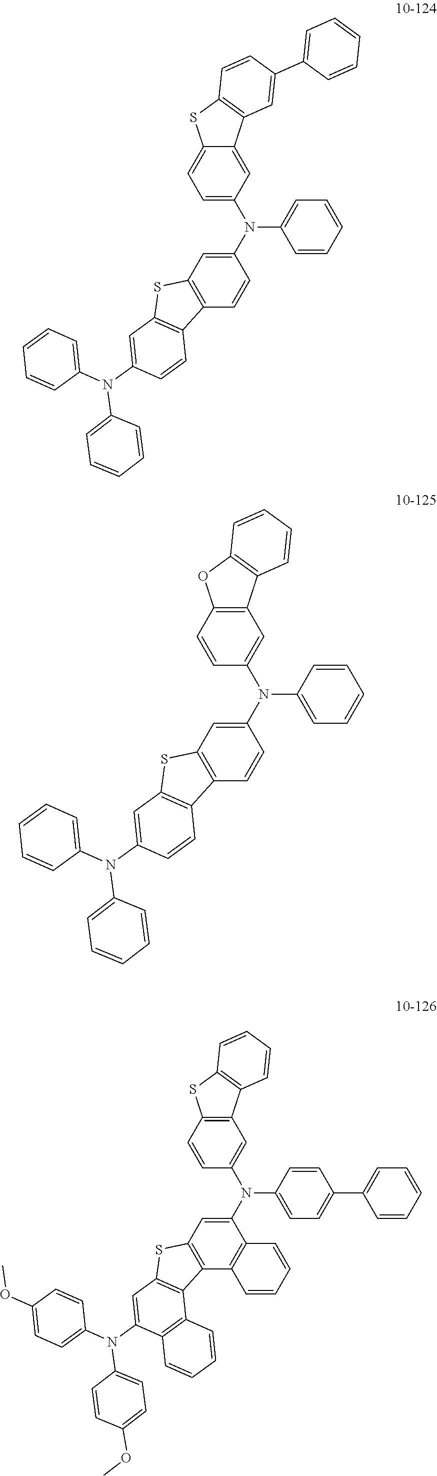

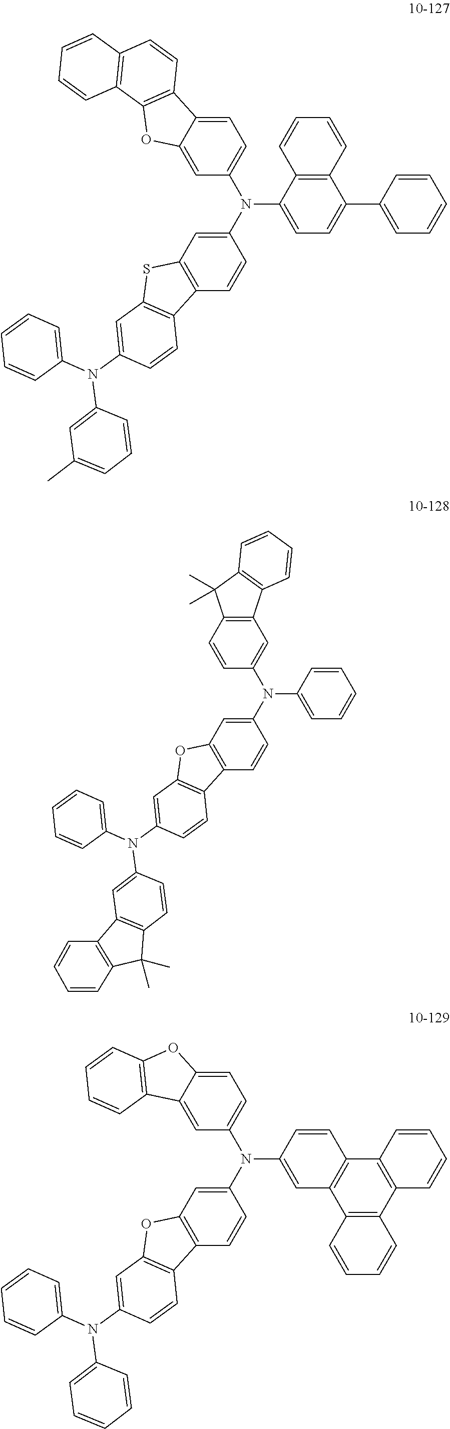

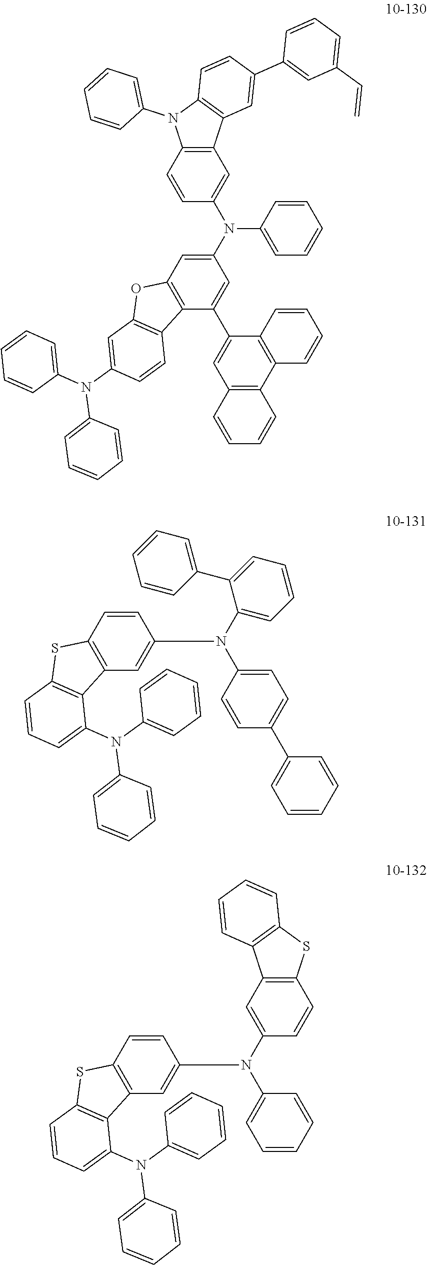

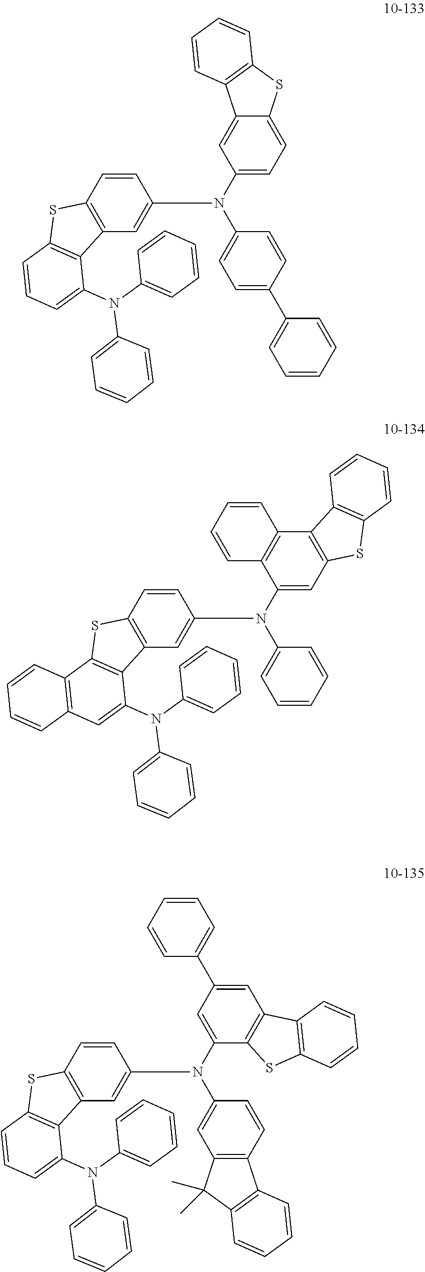

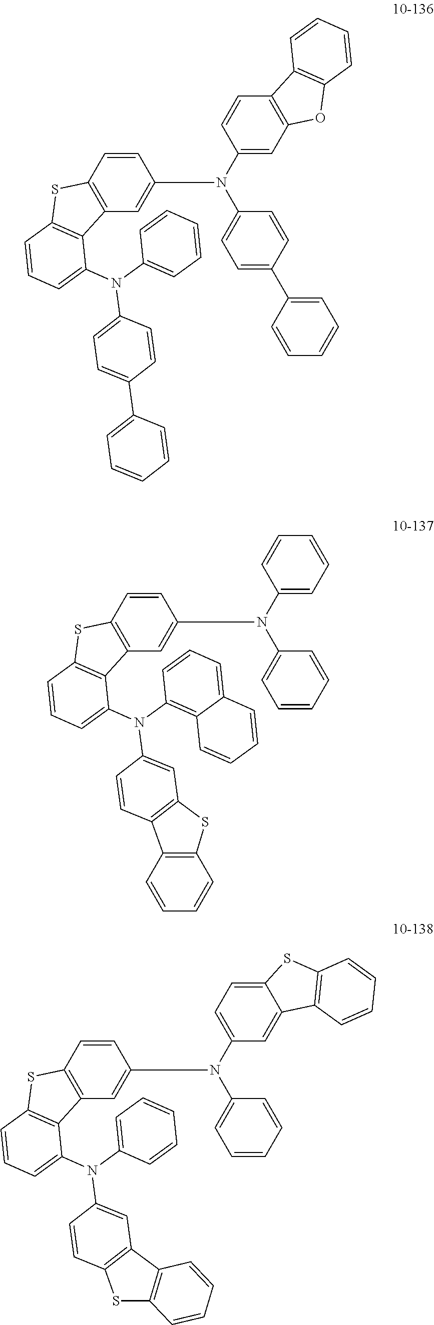

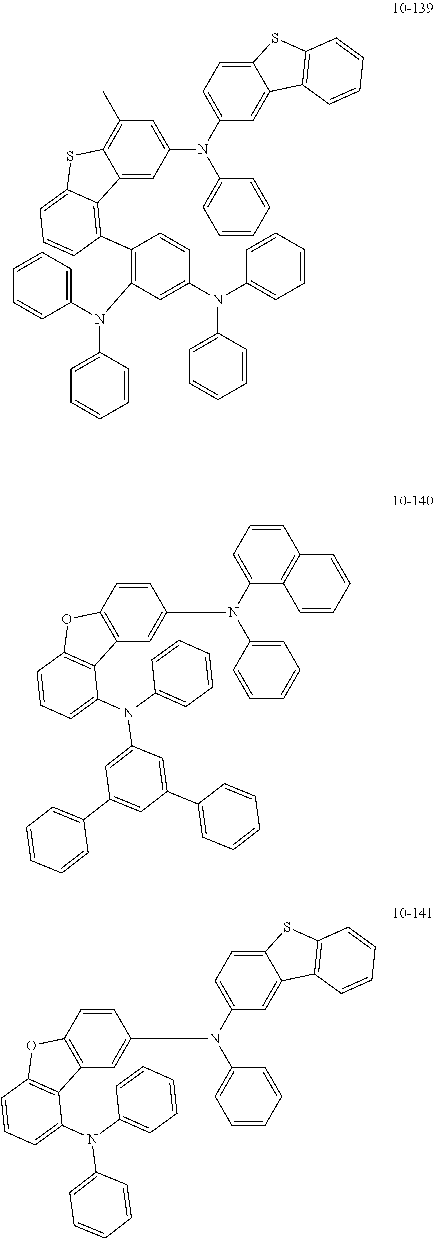

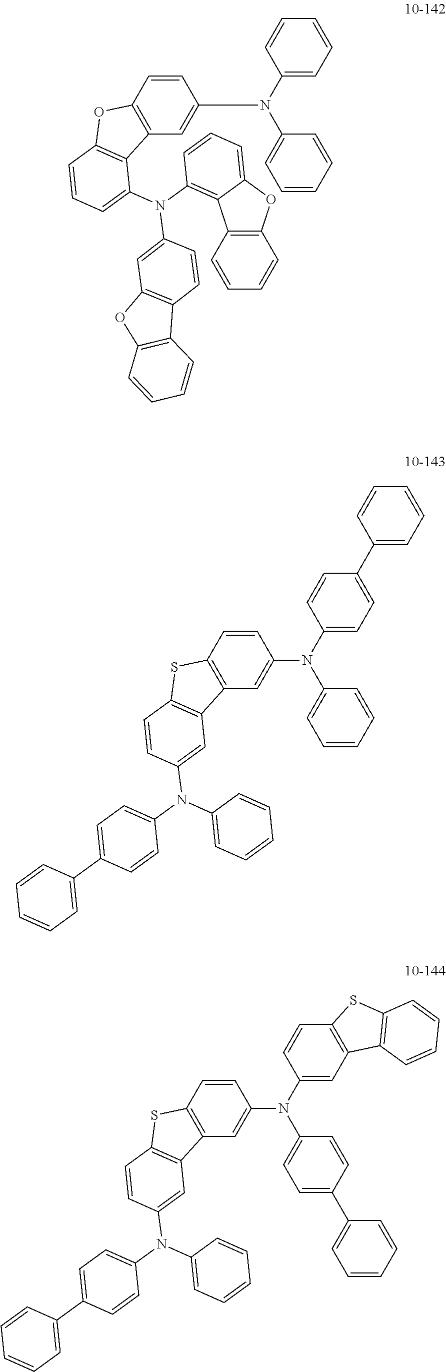

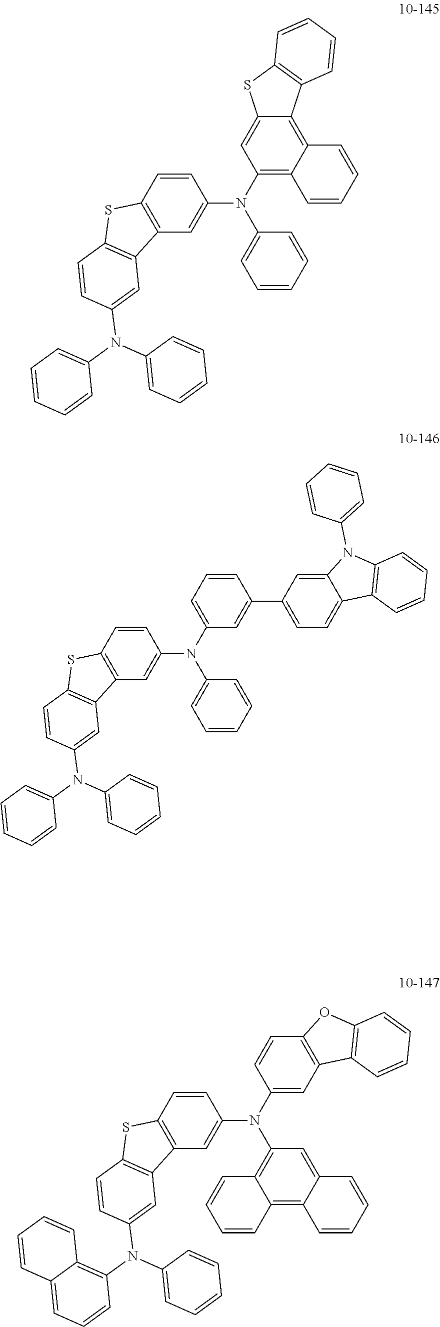

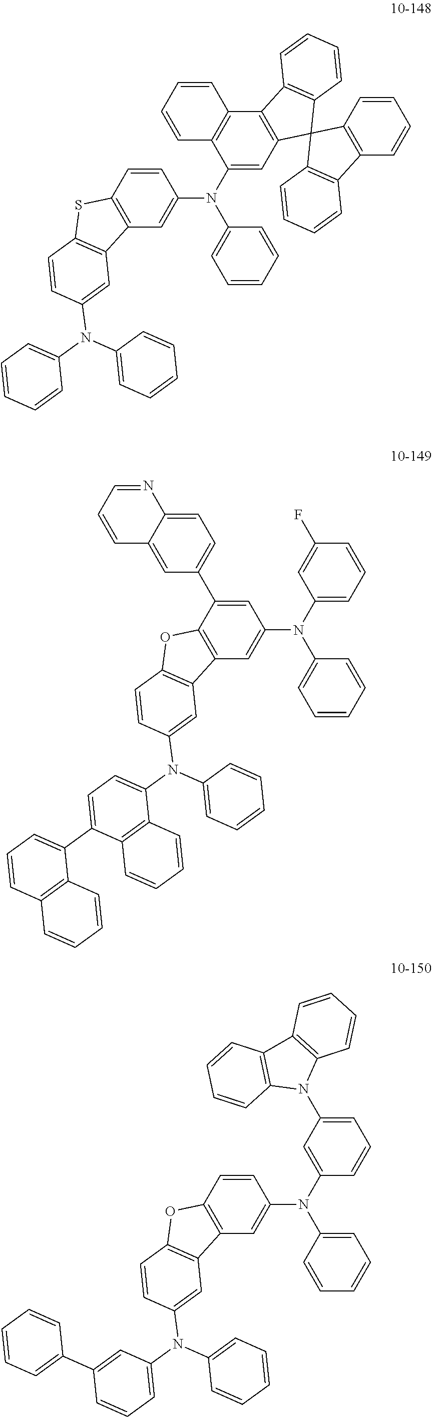

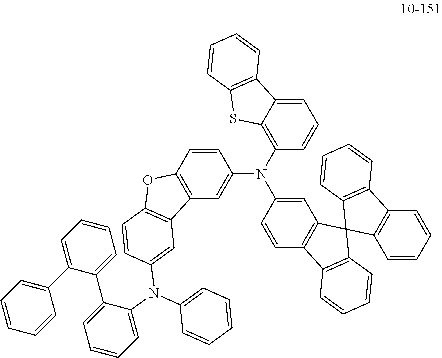

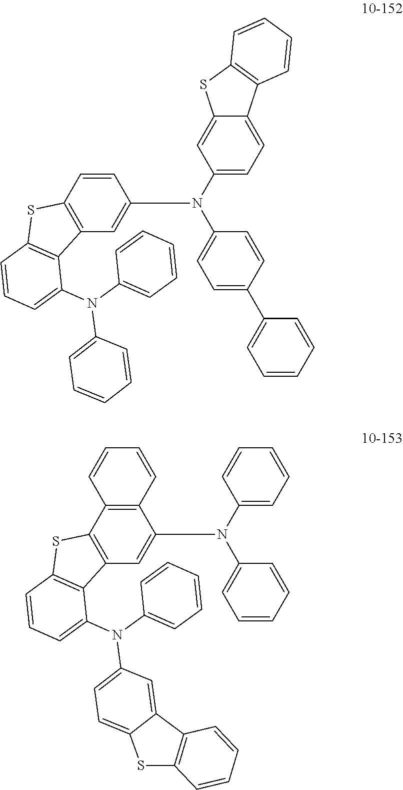

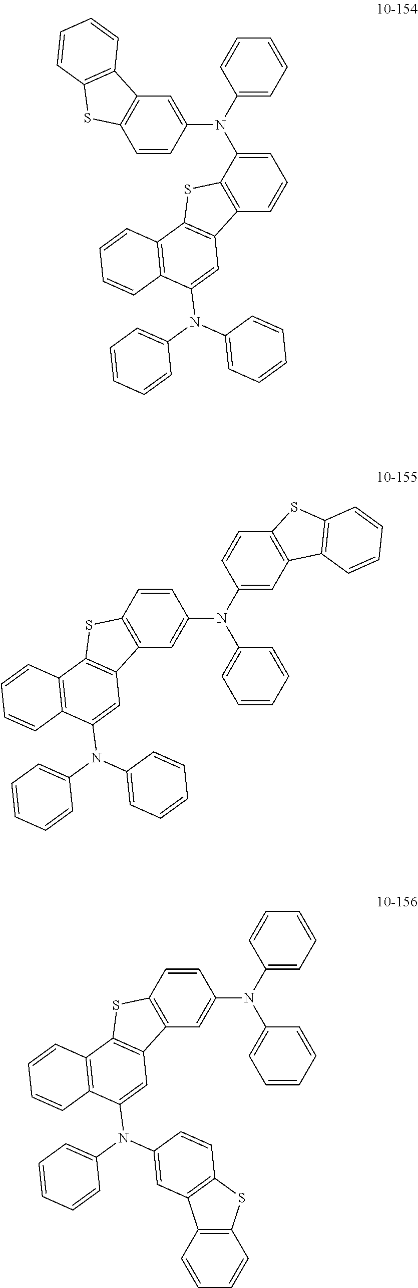

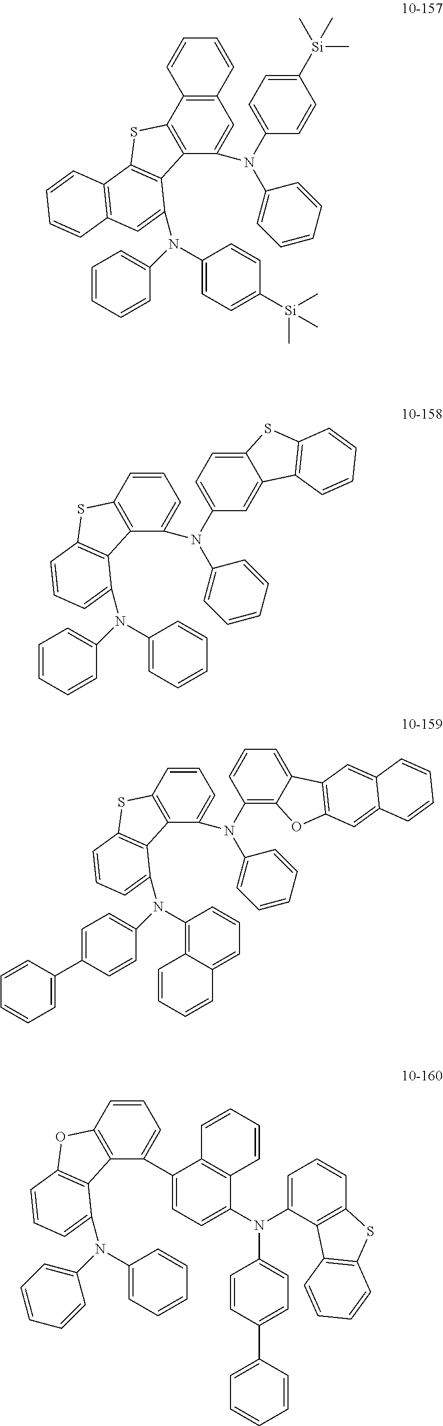

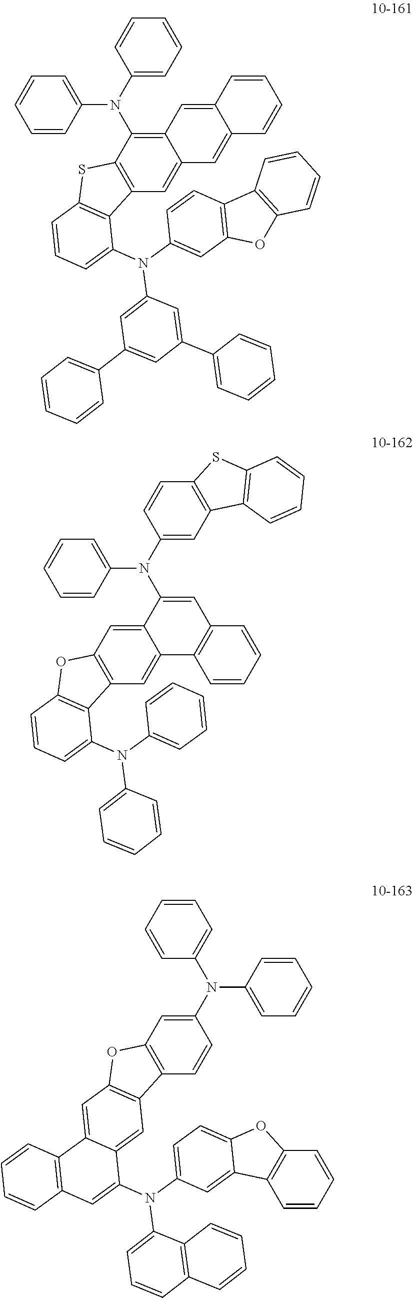

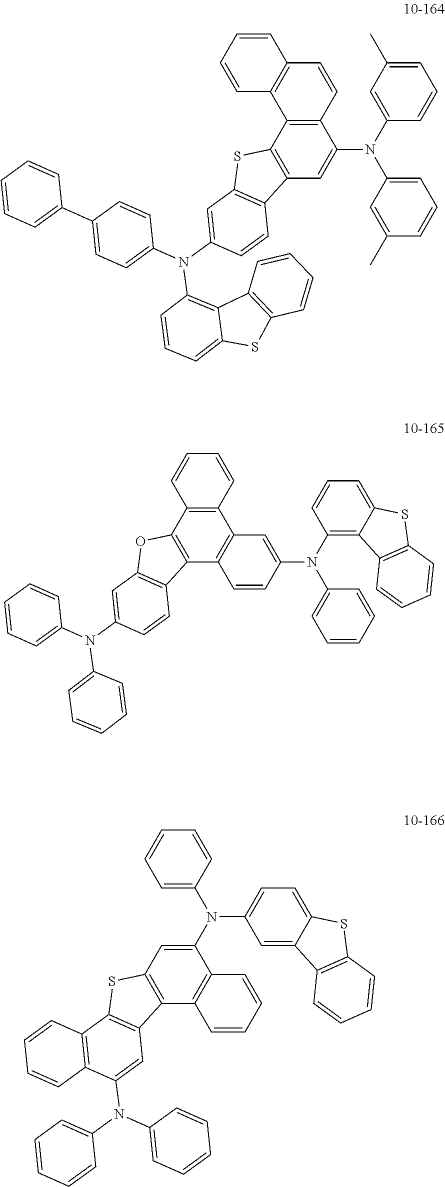

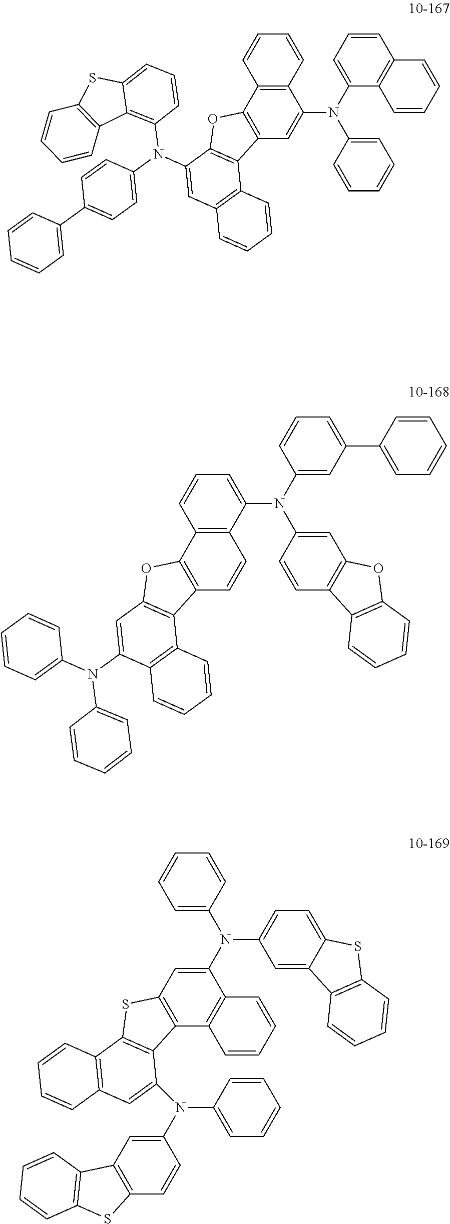

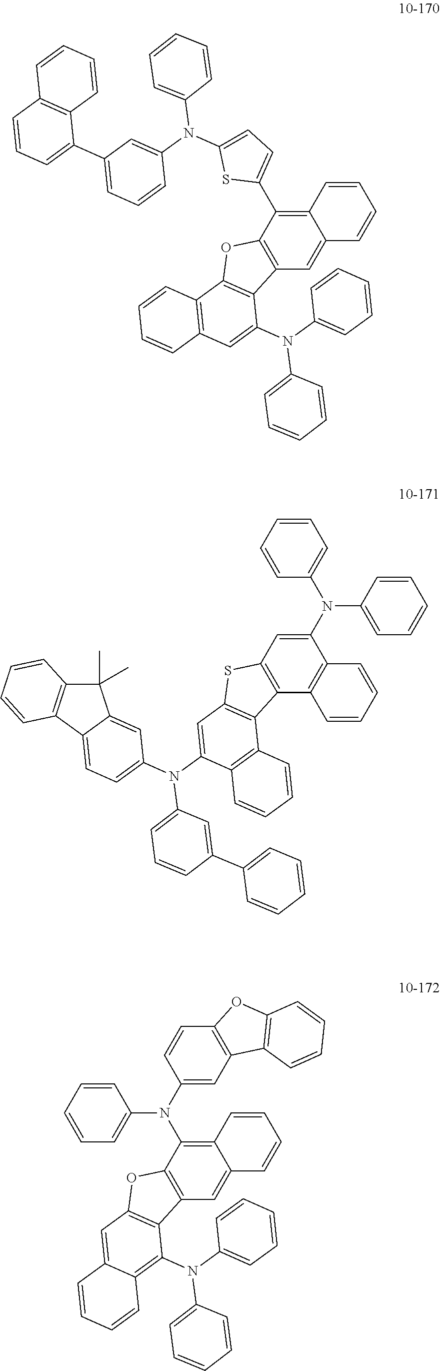

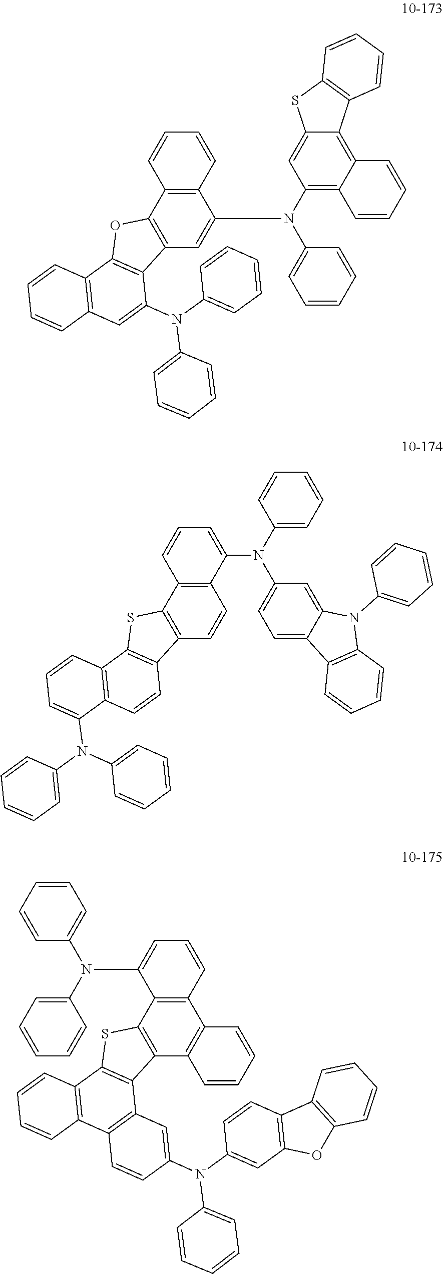

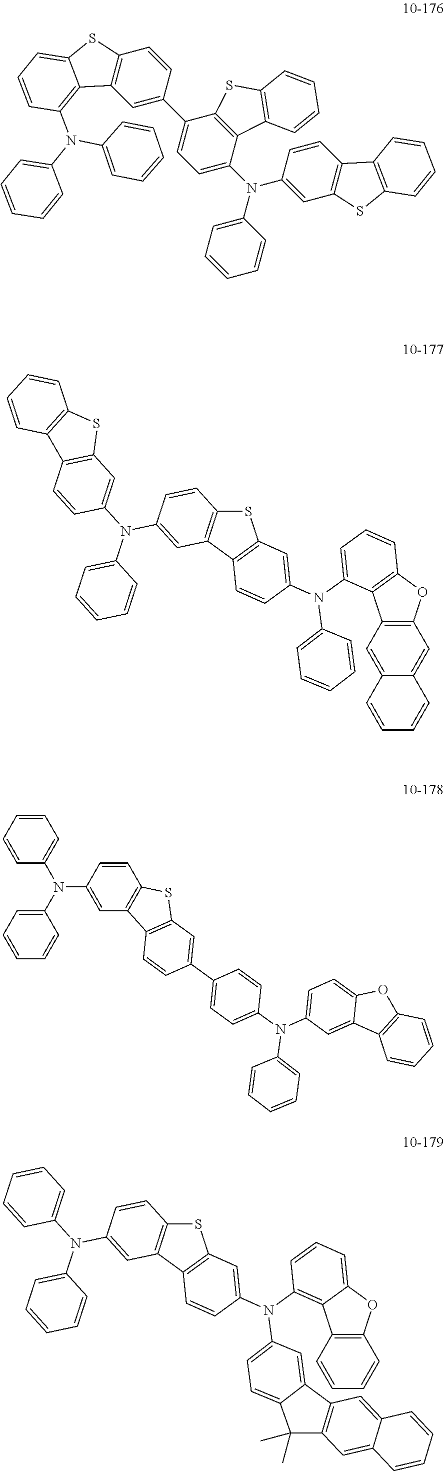

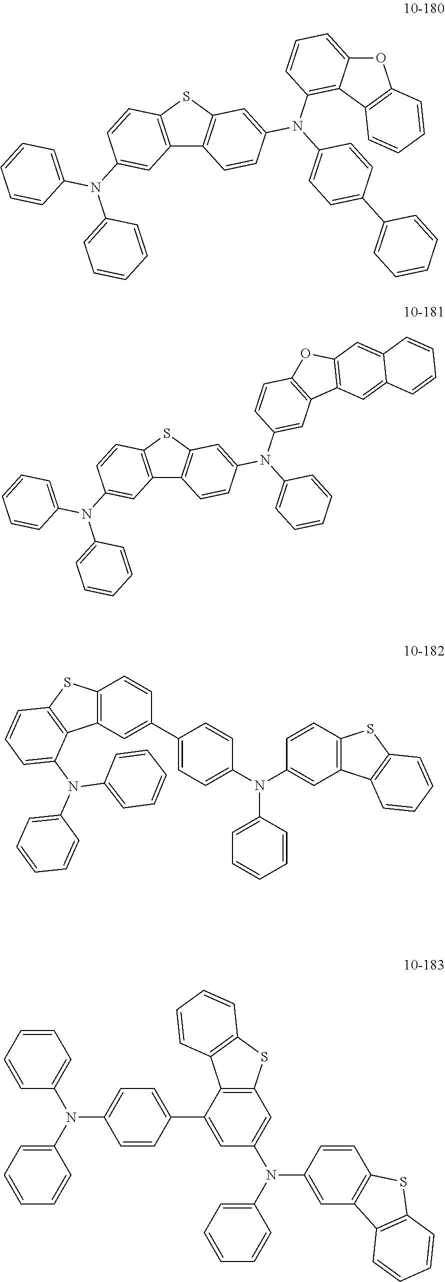

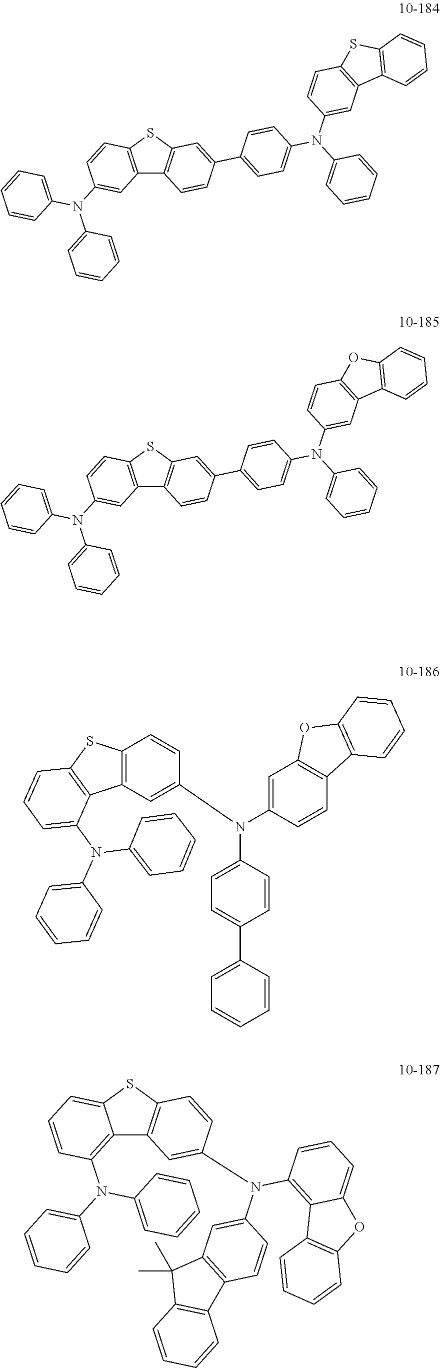

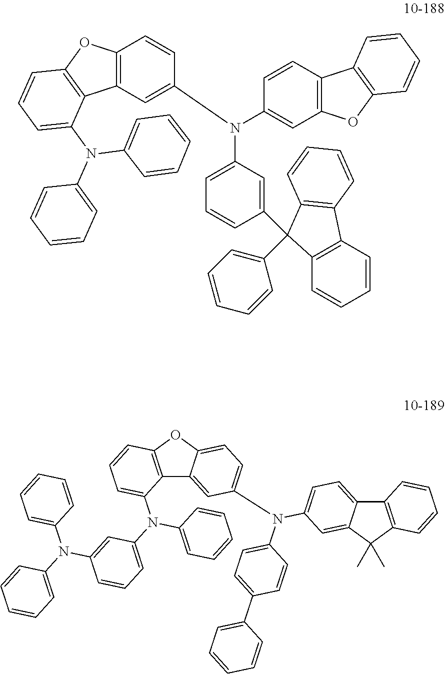

The fourth compound may include a radical of a compound represented by Formula B, be represented by Formula C or Formula D, and be one or more of the following Formula 11-1 to Formula 12-71.

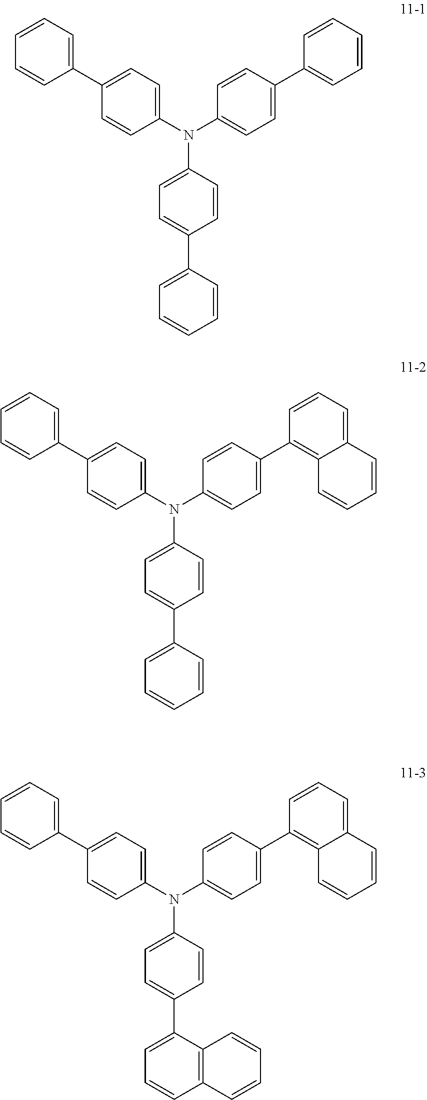

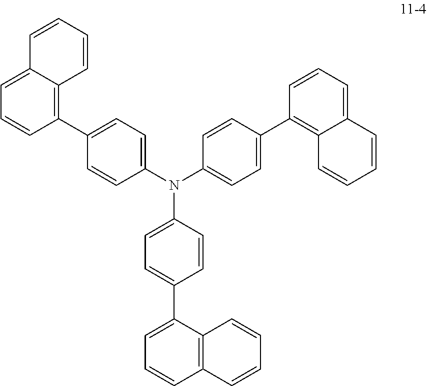

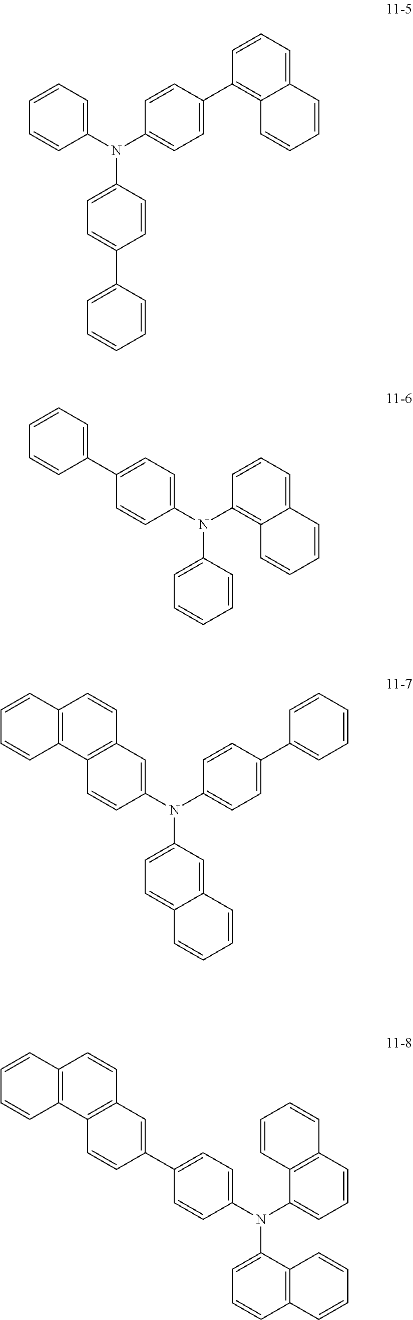

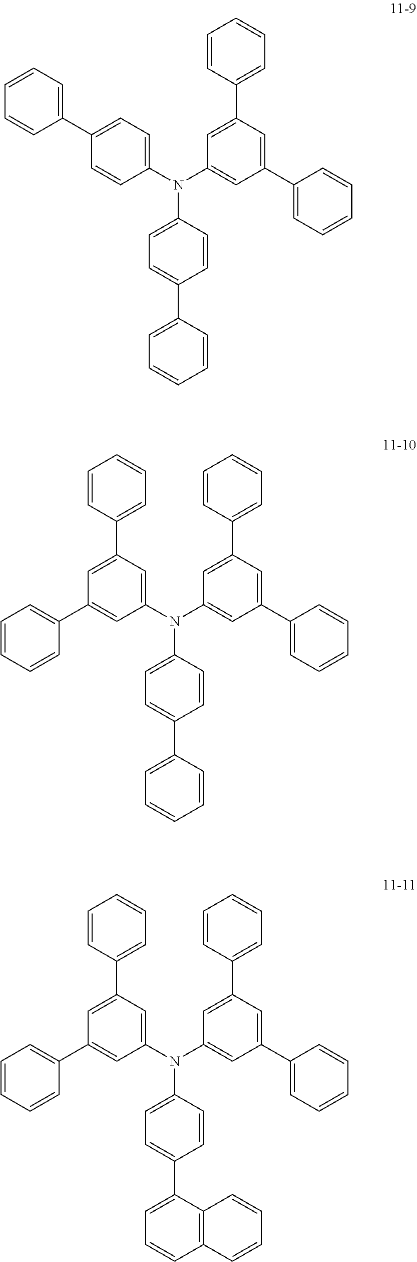

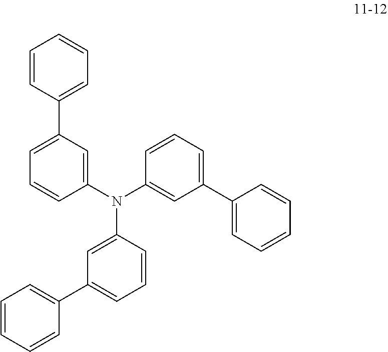

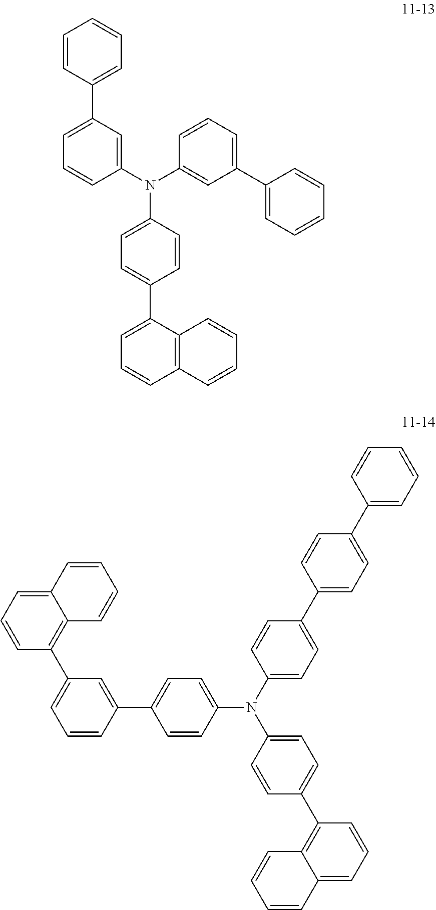

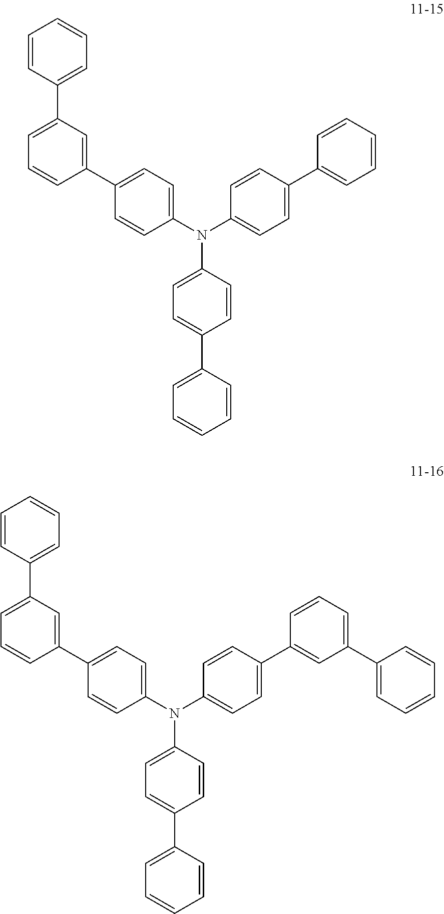

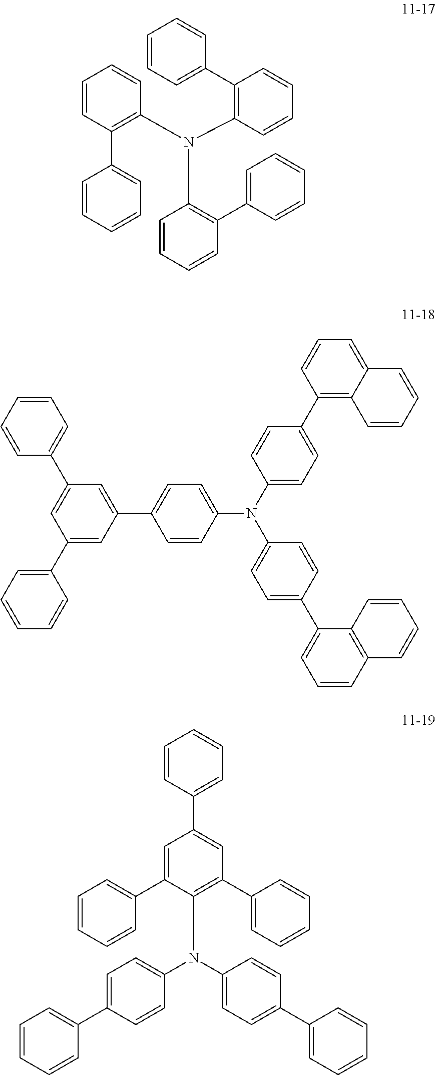

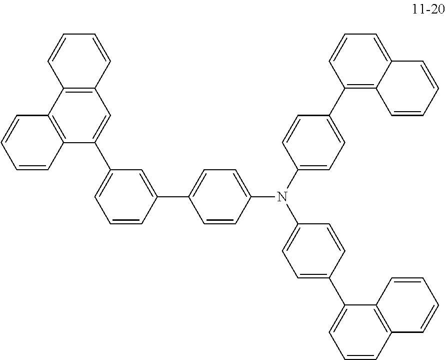

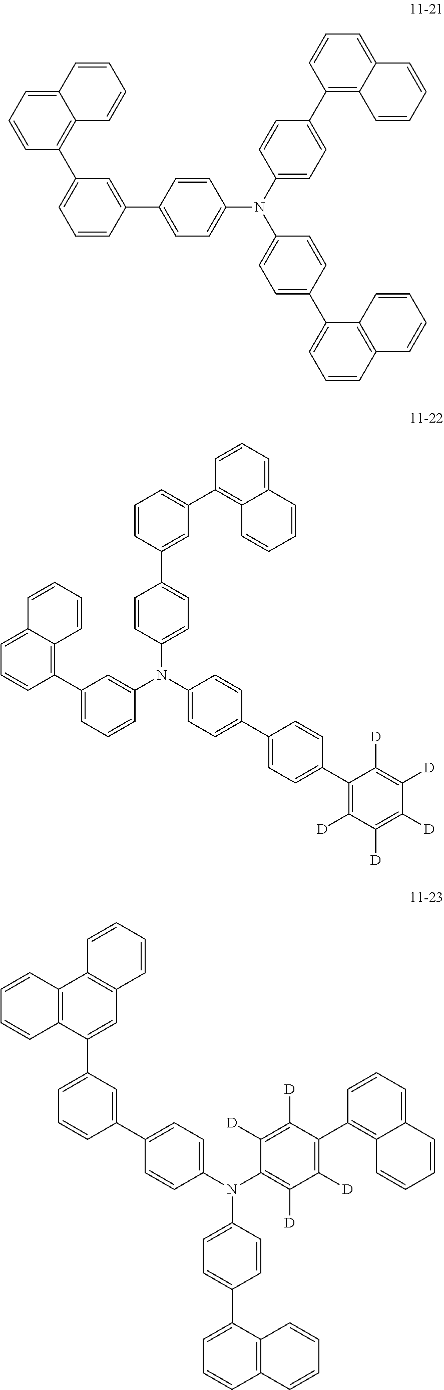

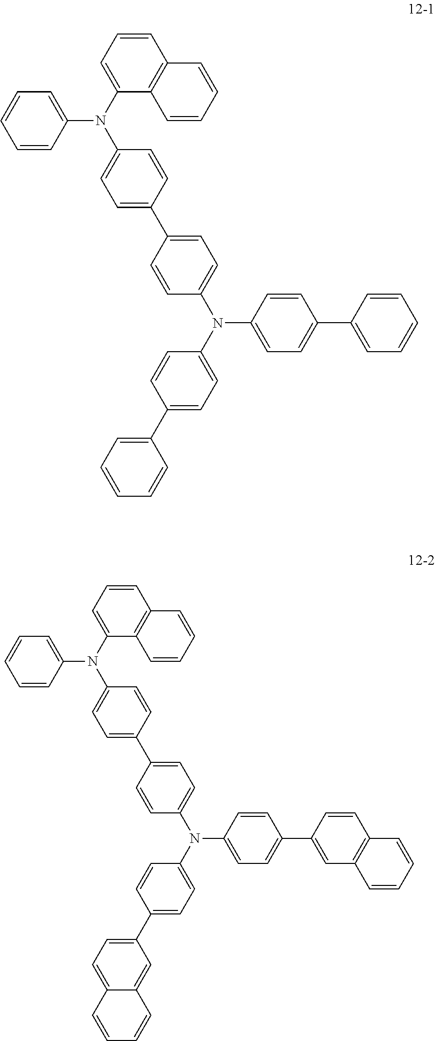

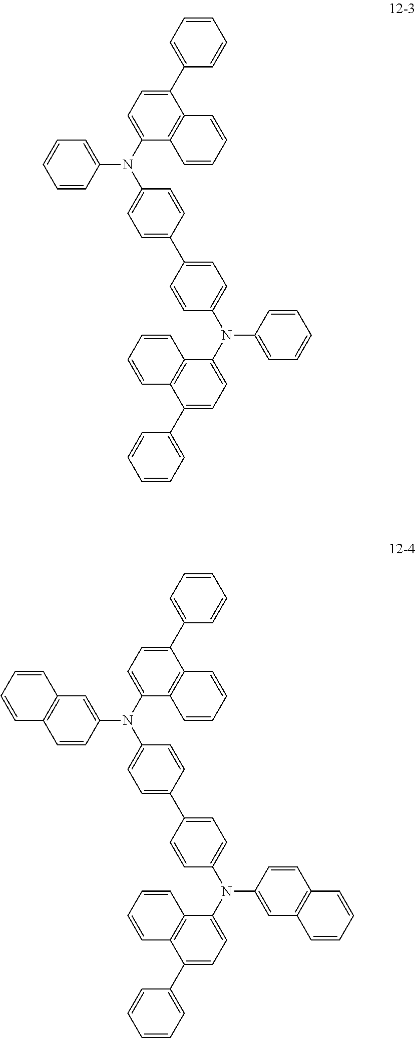

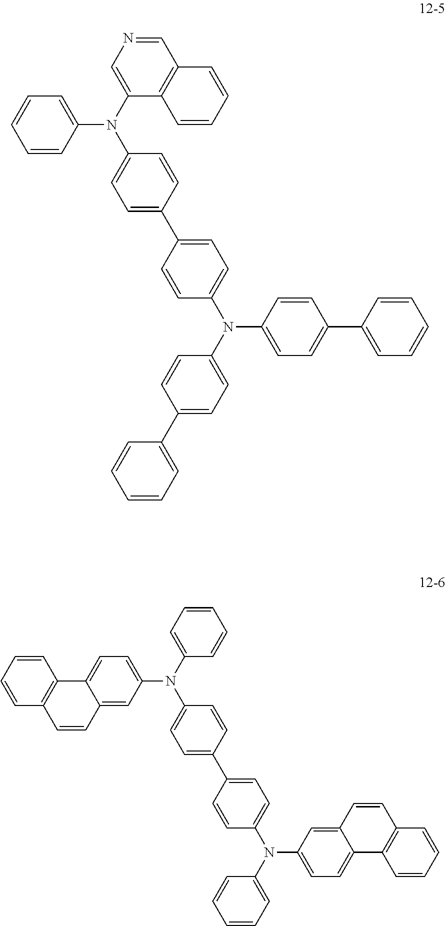

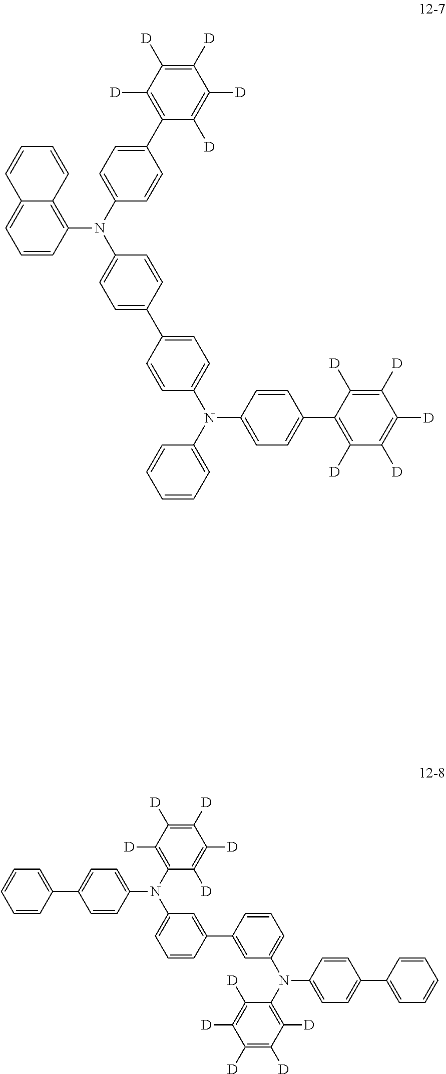

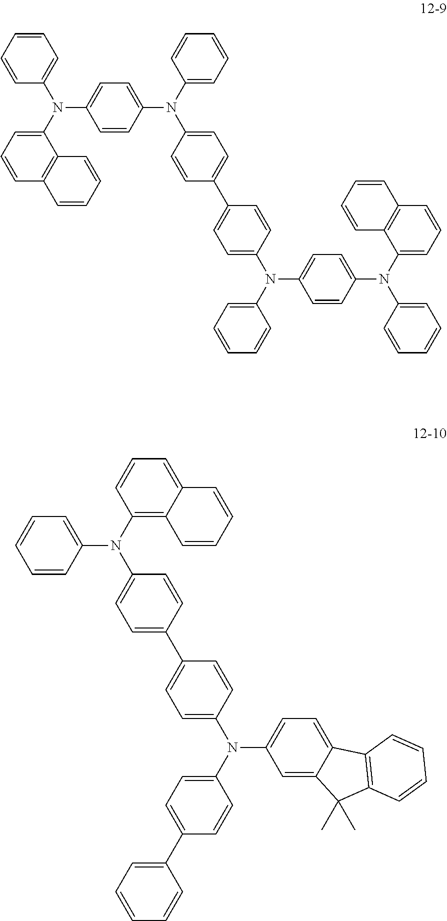

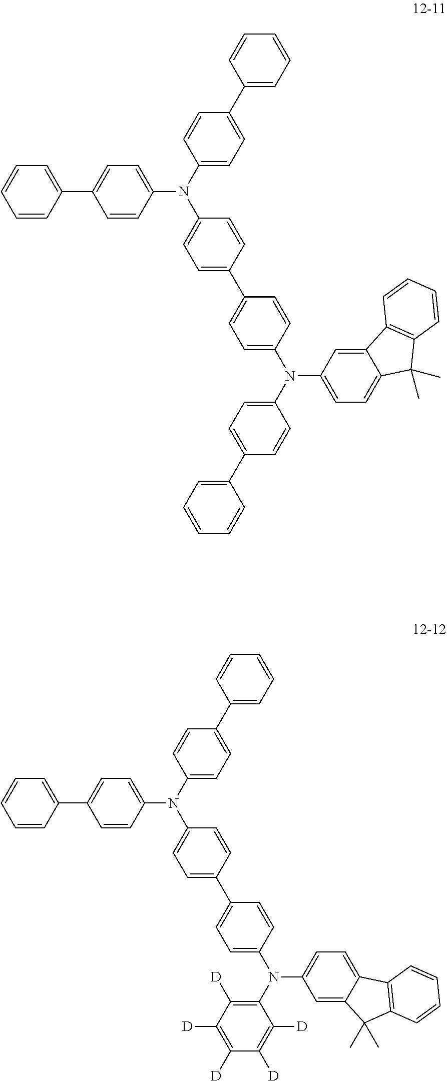

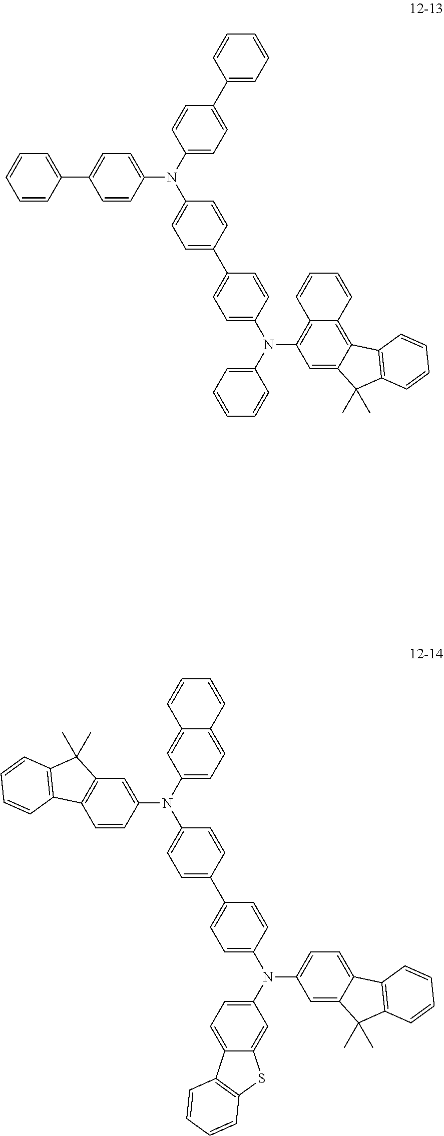

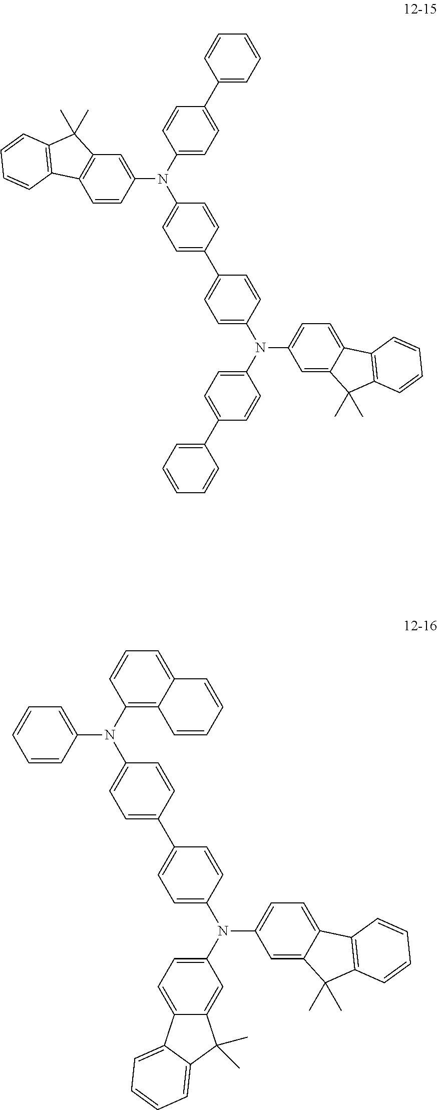

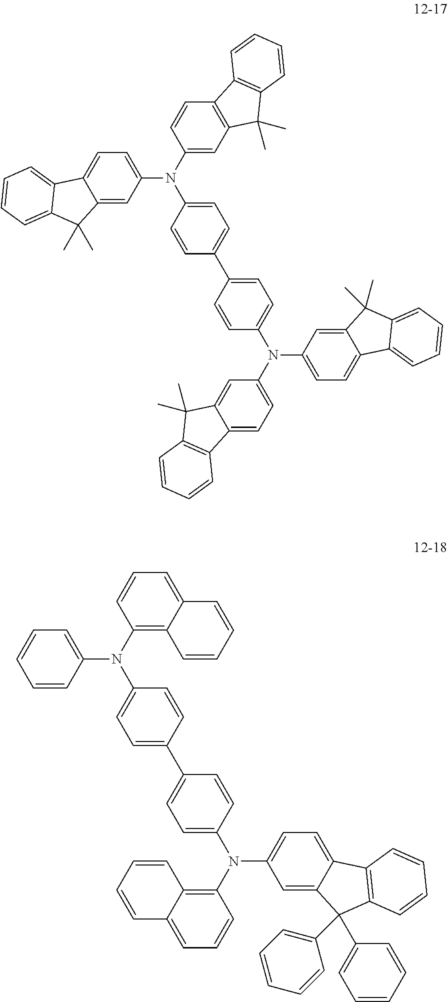

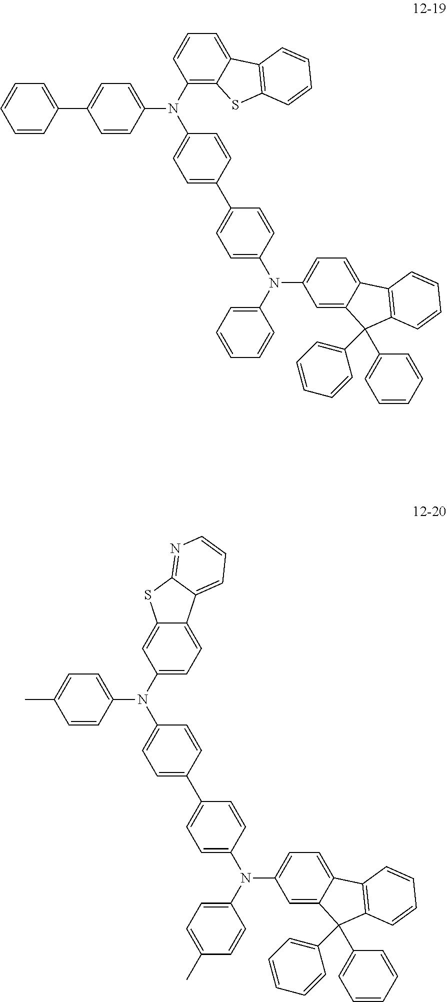

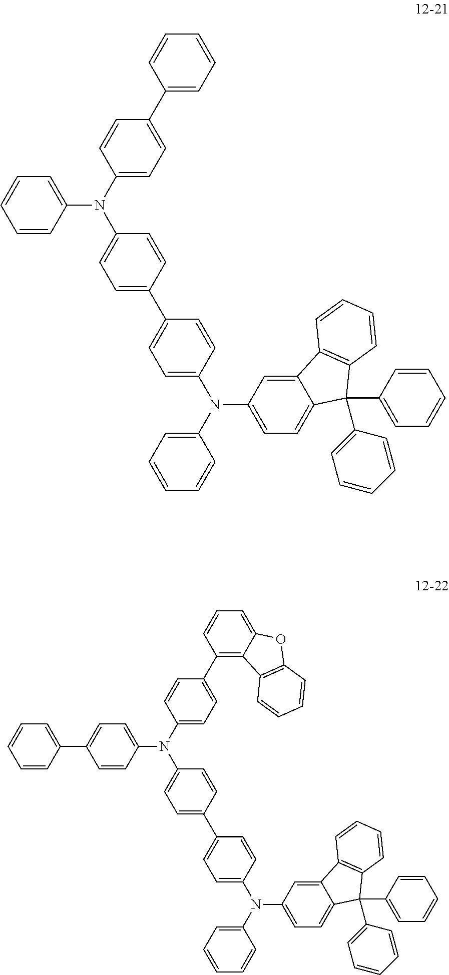

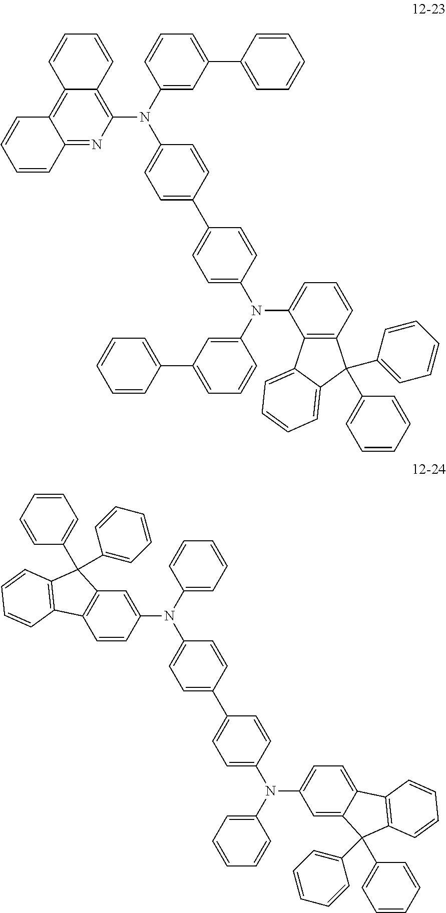

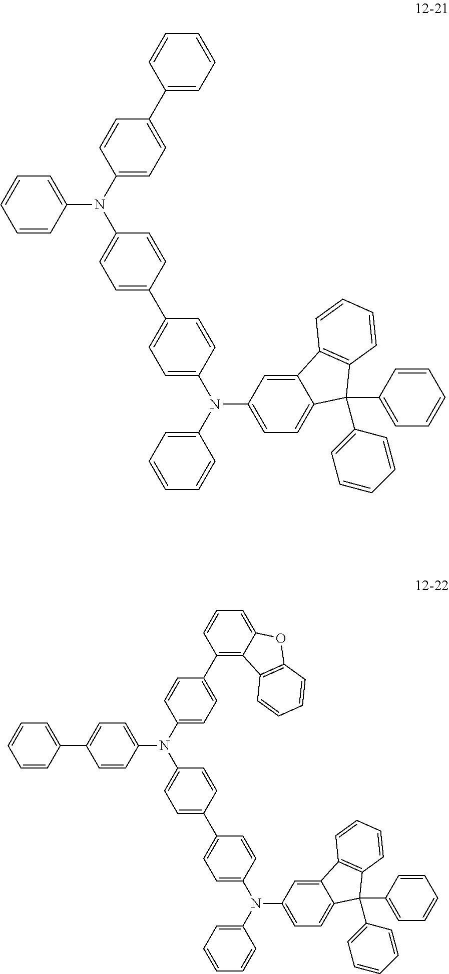

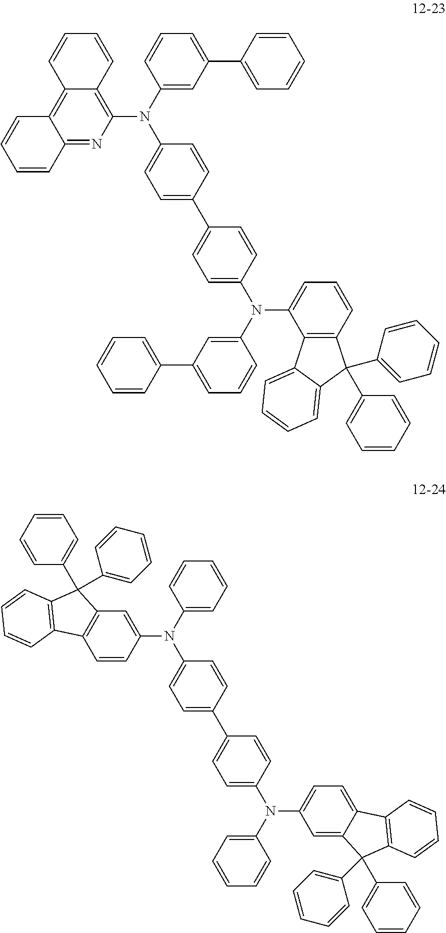

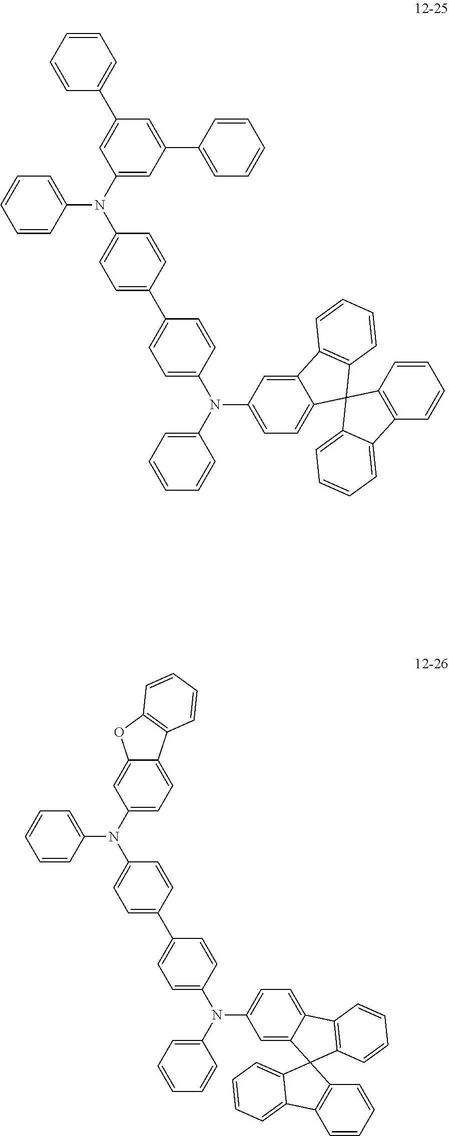

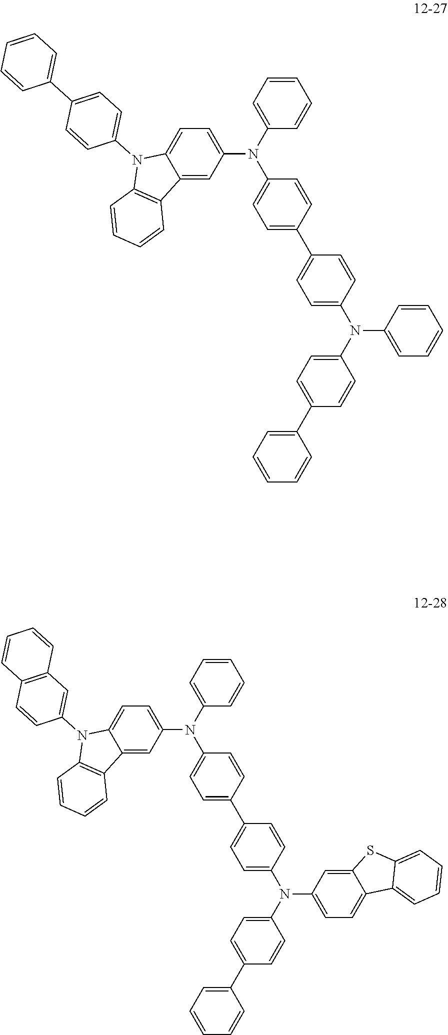

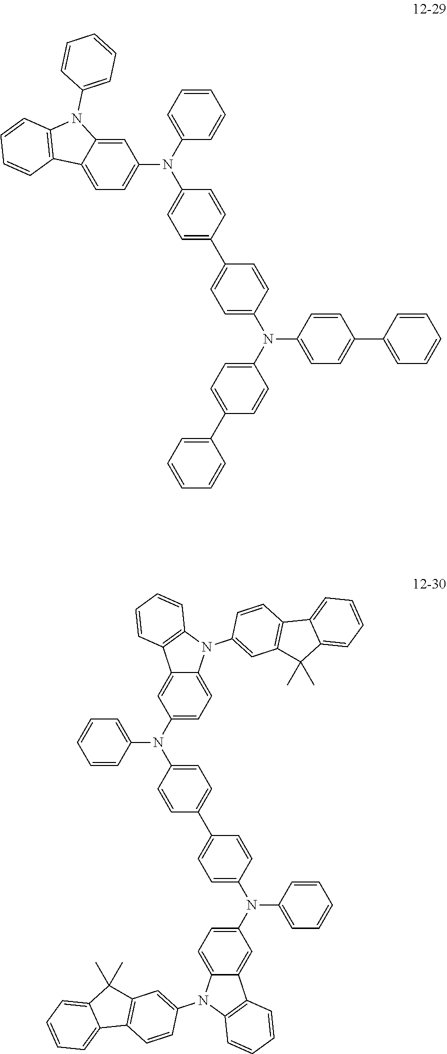

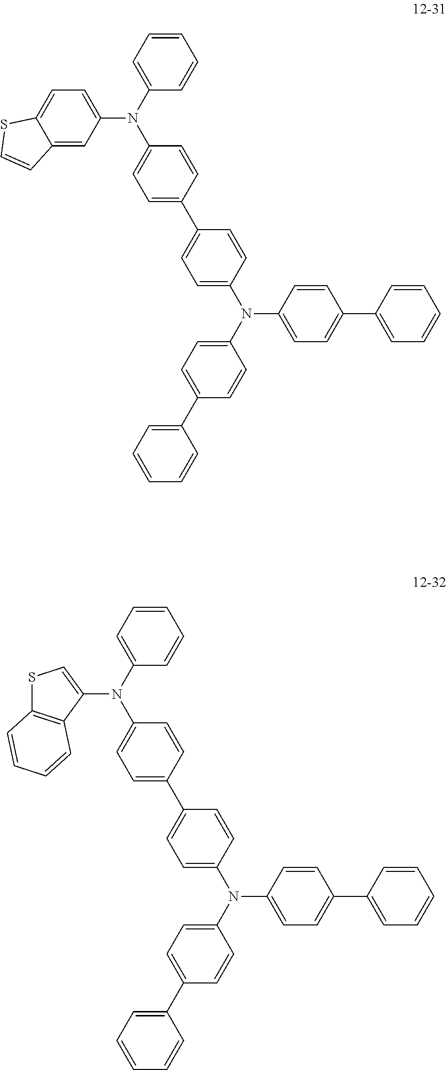

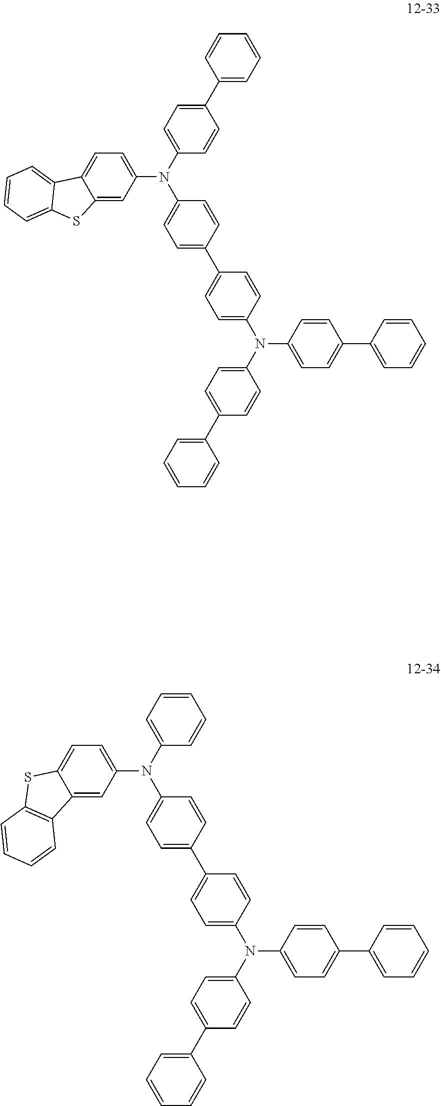

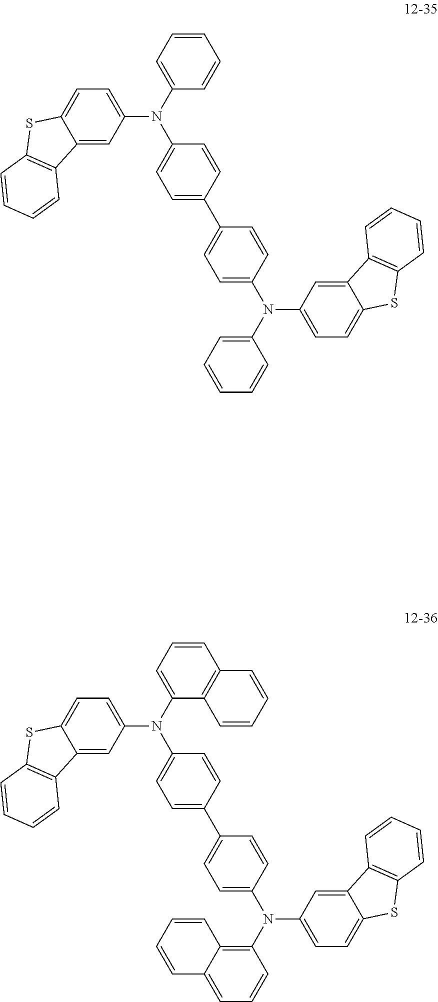

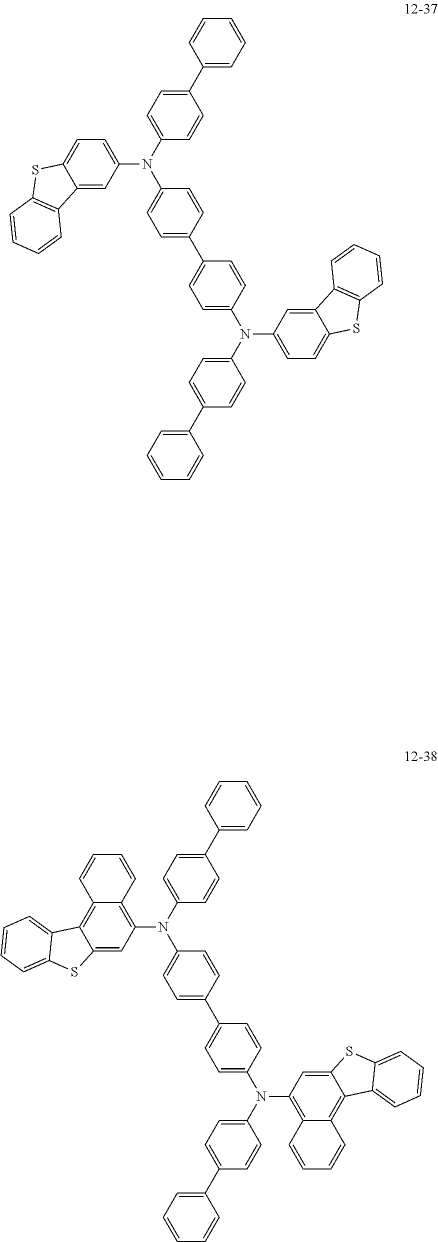

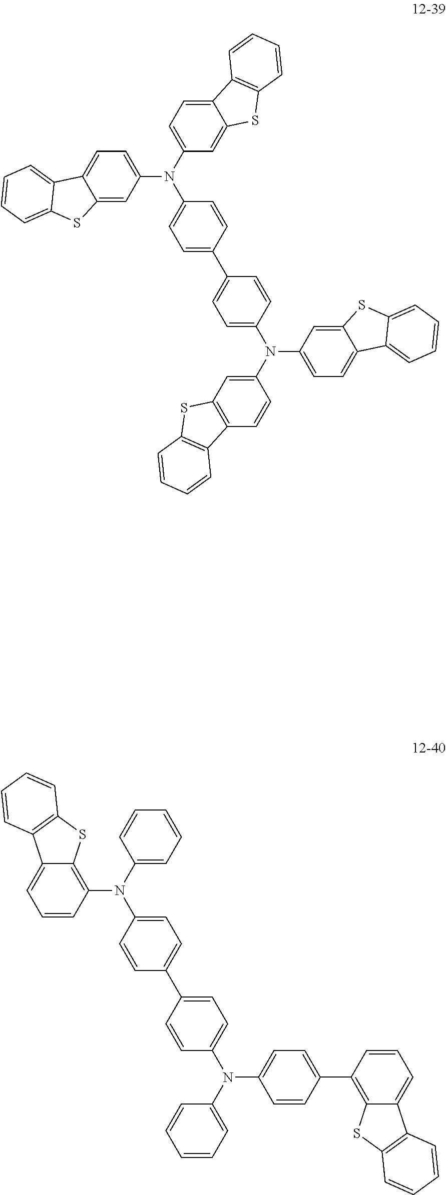

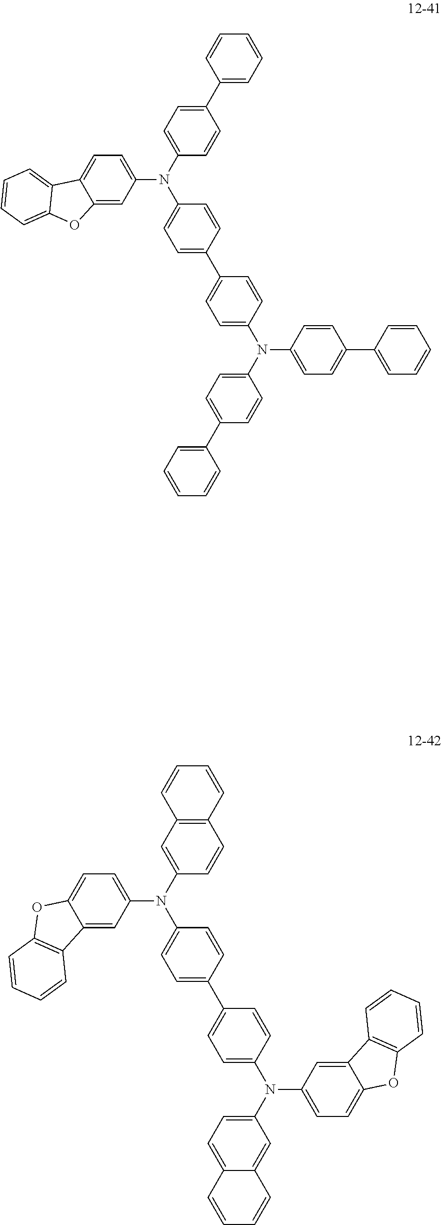

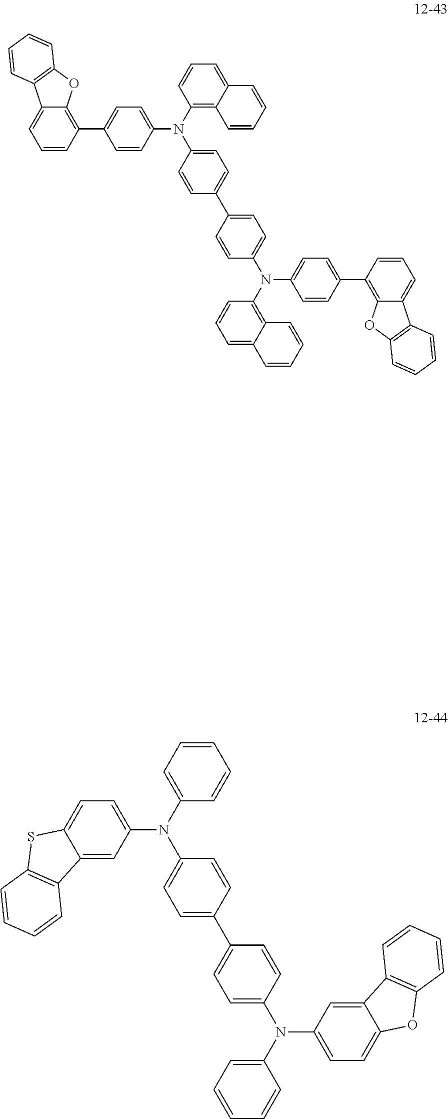

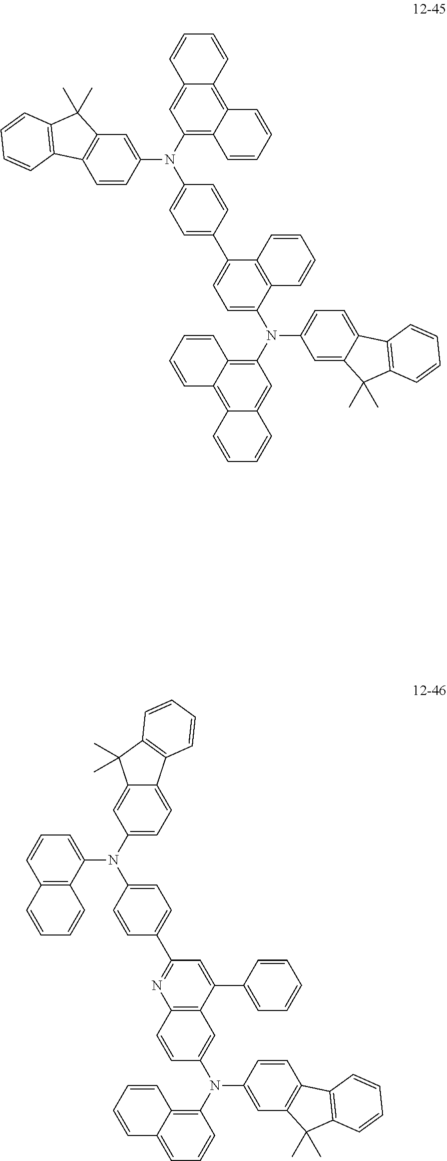

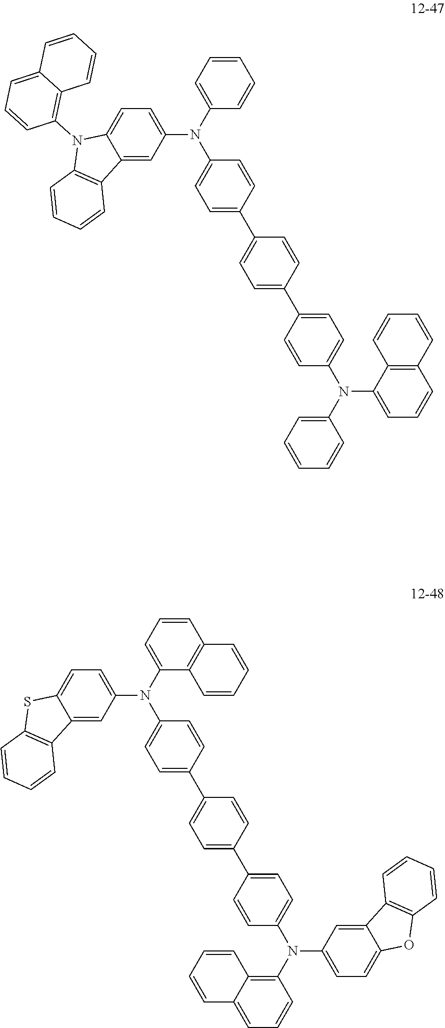

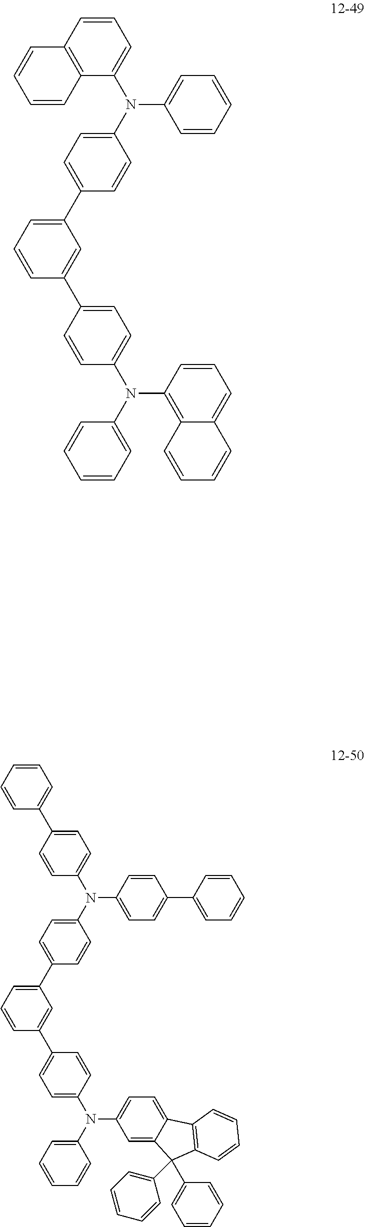

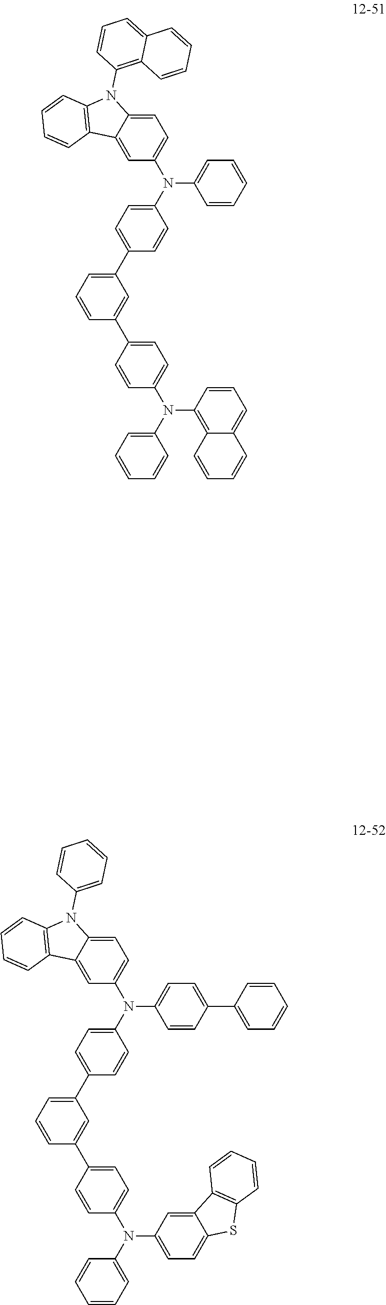

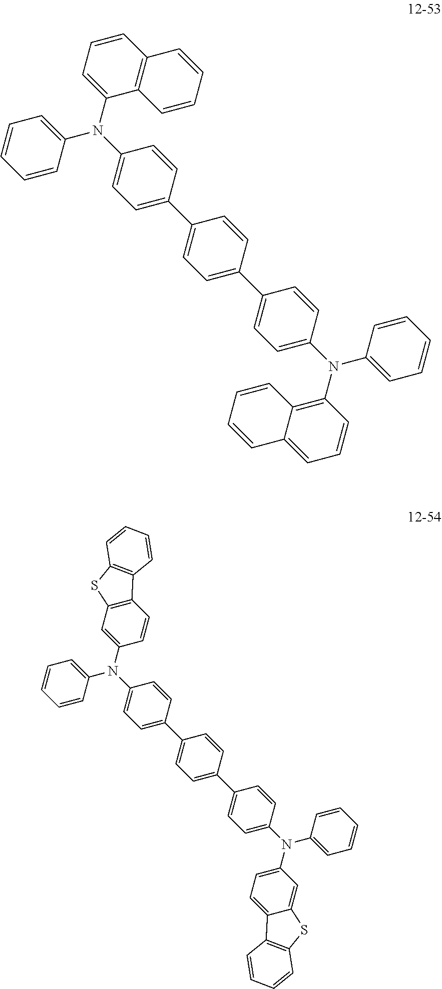

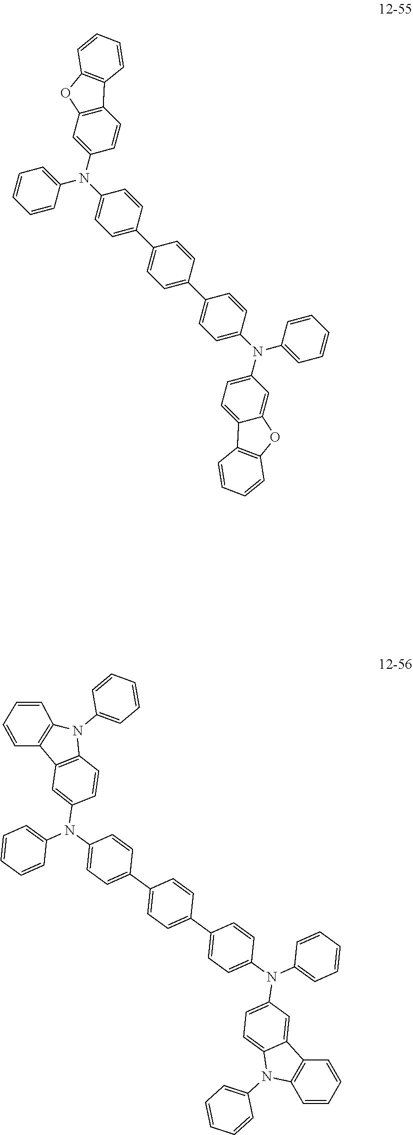

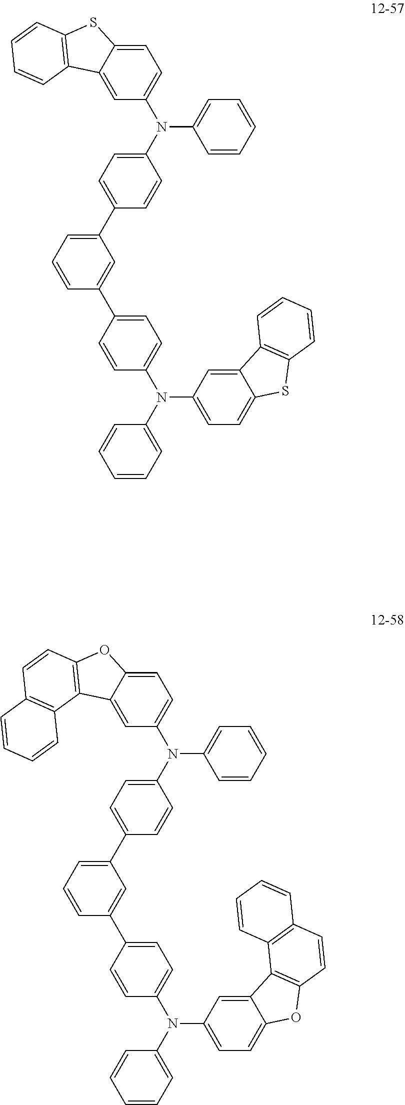

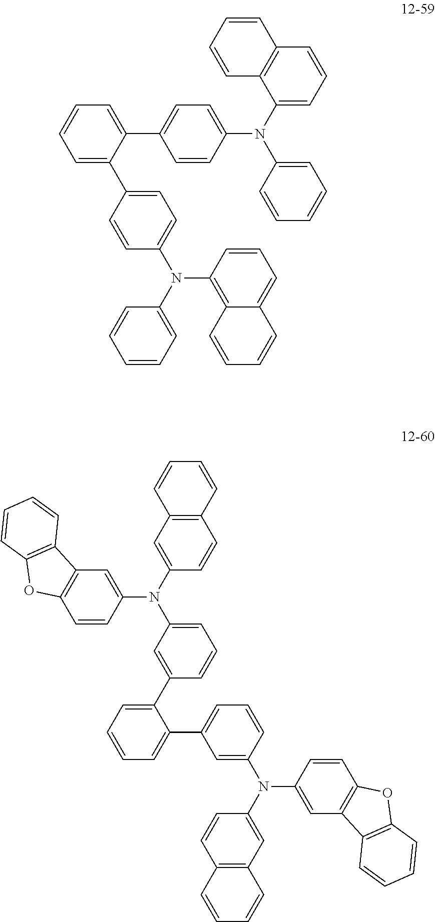

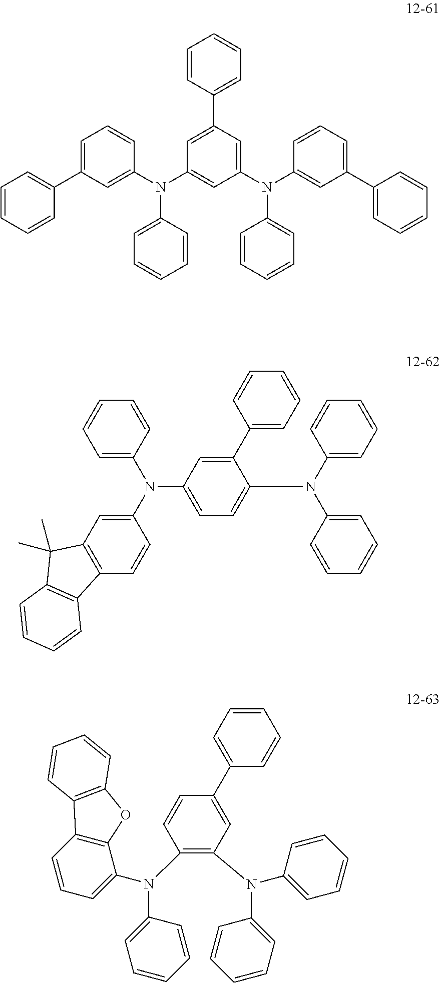

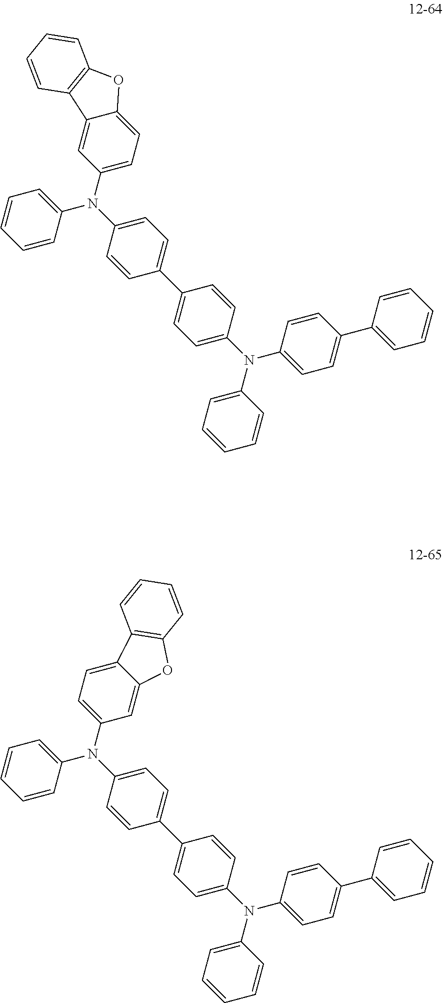

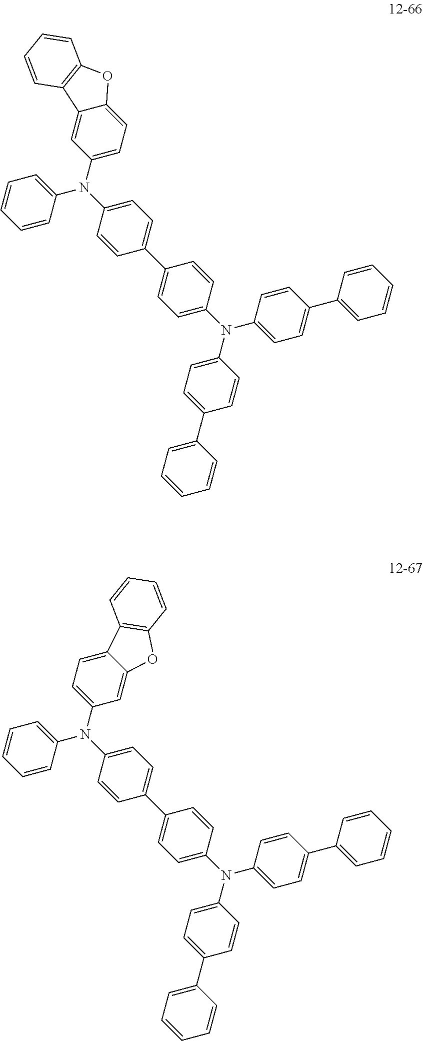

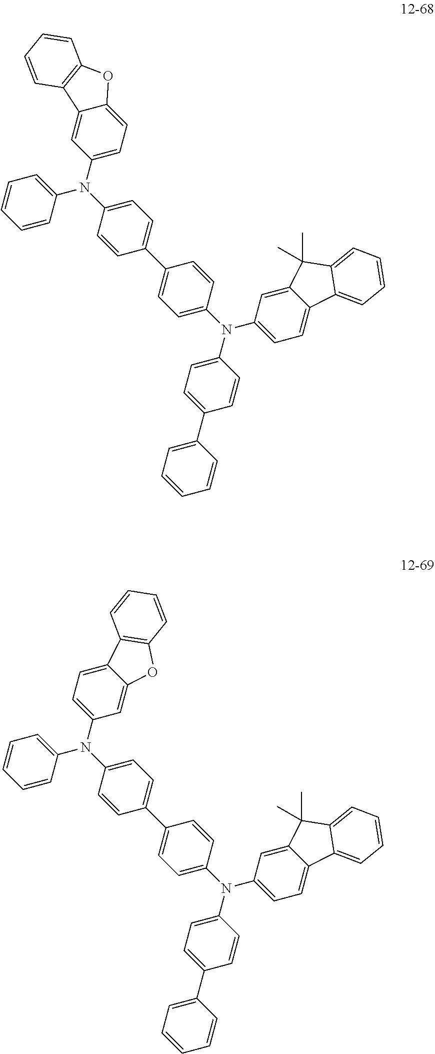

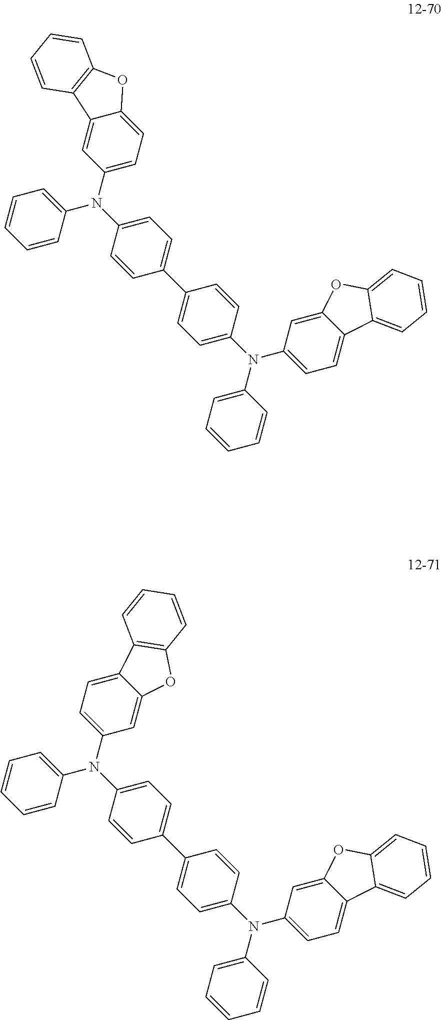

Regarding the sixth compound, what has been described above for the fourth compound may equally be applied, unless clearly stated otherwise.

The first doping material may be a P-type dopant. P-type dopants may be selected, for example, from quinodimethane compounds, azaindenofluorenedions, azaphenalenes, azatriphenylenes, I2, metal halides, transition metal halides, metal oxides, metal oxides including a metal from main group 3 or at least one transition metal, transition metal complexes, or complexes of Cu, Co, Ni, Pd, or Pt with ligands each including at least one oxygen atom as a bonding site. In another example, P-type dopants may be respectively selected from oxides of rhenium (Re), molybdenum (Mo), and tungsten (W). For example, P-type dopants may be respectively selected from Re₂O₇, MoO₃, WO₃, or ReO₃.

In another example, the first doping material may be represented by the following Formula E.

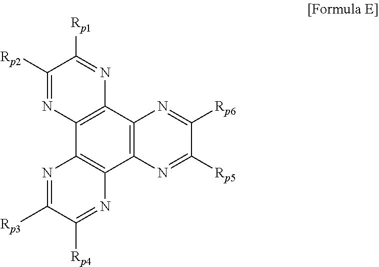

Hereinafter, Formula E will be described.

Each of Rₚ₁ to Rₚ₆ may be independently selected from the group consisting of hydrogen; a halogen group; a nitrile group; a nitro group; —SO₂R; —SOR; —SO₂NR₂; —SO₃R; a trifluoromethyl group; —COOR; —CONHR; —CONRR′; a C₁-C₃₀ alkoxyl group; a C₁-C₃₀ alkyl group; a C₂-C₂₀ alkenyl group; a C₂-C₃₀ heterocyclic group including at least one hetero atom of O, N, S, Si, or P; a fluorenyl group; a C₆-C₃₀ aryl group; a fused ring group of a C₃-C₃₀ aliphatic ring and C₆-C₃₀ aromatic ring; or —NRR′.

R and R′ may be respectively selected from the group consisting of a C₁-C₃₀ alkyl group; a fluorenyl group; a C₆-C₃₀ aryl group; a fused ring group of a C₃-C₃₀ aliphatic ring and a C₆-C₃₀ aromatic ring; or a C₂-C₃₀ heterocyclic group including at least one hetero atom of O, N, S, Si, or P.

In Formula E, each of the aryl group, the fluorenyl group, the heterocyclic group, the fused ring group, the alkyl group, the alkenyl group, the alkynyl group, and the alkoxyl group may be substituted with one or more substituents selected from the group consisting of deuterium; a halogen group; a C₁-C₂₀ alkoxyl group; a C₁-C₂₀ alkyl group; a C₂-C₂₀ alkenyl group; a C₂-C₂₀ alkynyl group; a C₆-C₂₅ aryl group; a C₆-C₂₅ aryl group substituted with deuterium; a fluorenyl group; a C₂-C₂₀ heterocyclic group; or a C₃-C₂₀ cycloalkyl group.

In another example, the first doping material may be selected from the following E-1 to E-4.

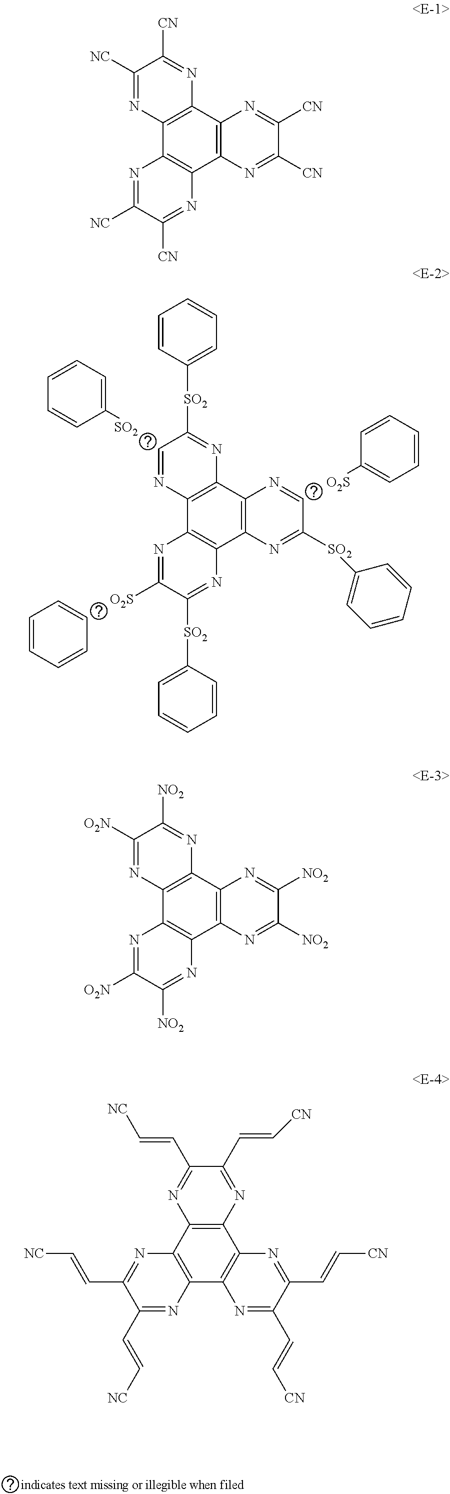

In another example, the first doping material may be selected from the following E-5 to E-14.

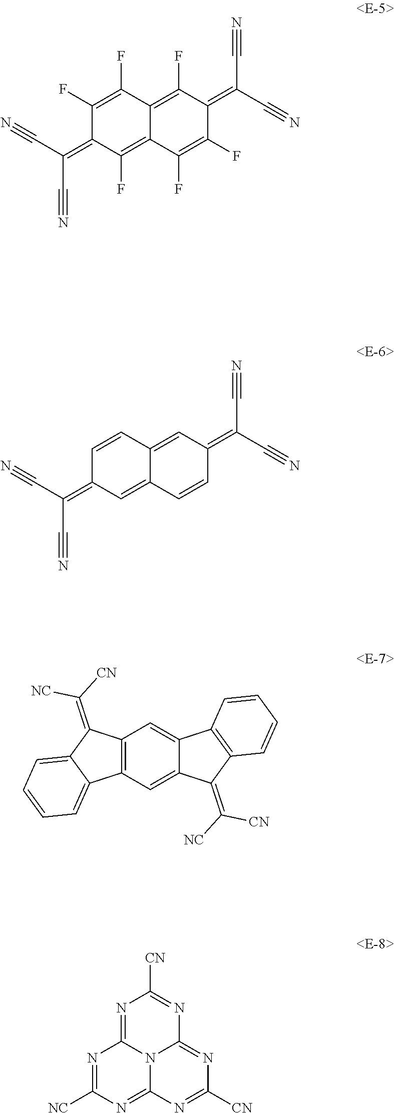 [SMILES: N#CC(C#N)=c1c(F)c(F)c2c(F)c(=C(C#N)C#N)c(F)c(F)c2c1F ; N#CC(C#N)=c1ccc2cc(=C(C#N)C#N)ccc2c1 ; N#CC(C#N)=C1c2ccccc2-c2cc3c(cc21)-c1ccccc1C3=C(C#N)C#N ; N#CC1=NC2=NC(C#N)=NC3=NC(C#N)=NC(=N1)N23]

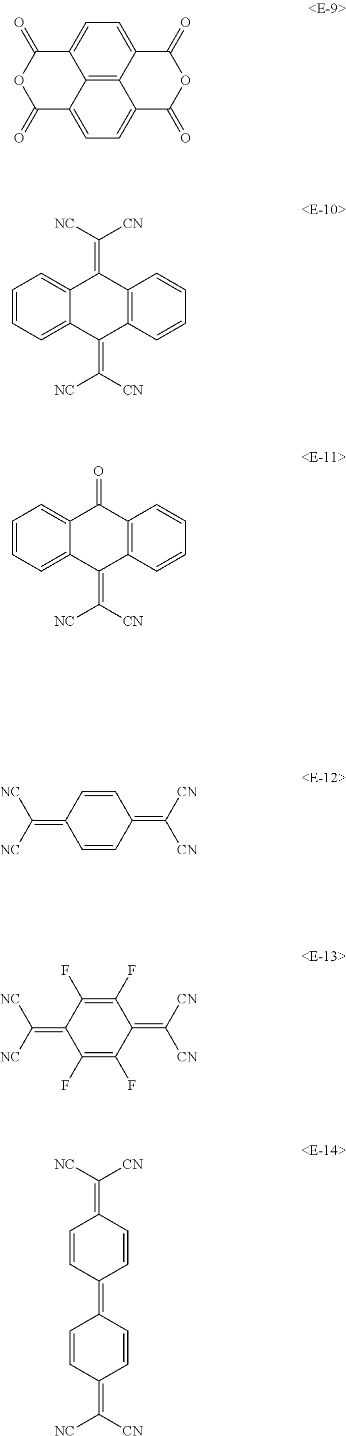 [SMILES: O=C1OC(=O)c2ccc3c4c(ccc1c24)C(=O)OC3=O ; N#CC(C#N)=c1c2ccccc2c(=C(C#N)C#N)c2ccccc12 ; N#CC(C#N)=C1c2ccccc2C(=O)c2ccccc21 ; N#CC(C#N)=c1ccc(=C(C#N)C#N)cc1 ; N#CC(C#N)=c1c(F)c(F)c(=C(C#N)C#N)c(F)c1F ; N#CC(C#N)=c1ccc(=c2ccc(=C(C#N)C#N)cc2)cc1]

Regarding the second doping material to the fourth doping material, what has been described above for the first doping material may equally be applied, unless clearly stated otherwise.

In some embodiments of the present disclosure, the organic material layer 130 includes the first stack 141, the second stack 142, and the third stack 143. The first stack 141 may include the first hole transport region 1411, the first emission layer 1412, and the first electron transport region 1413. In these embodiments, the first hole transport region 1411 may include the first hole transport layer 1411a and the first auxiliary emission layer 1411b, the first hole transport layer 1411a or the first auxiliary emission layer 1411b may include the first compound represented by Formula 1, the thickness of the first hole transport layer 1411a may be from 250 Å to 700 Å, and 10% to 50% of the thickness of the first hole transport layer 1411a may be doped with the first doping material.

In some embodiments of the present disclosure, the organic material layer 130 may include the first stack 141, the second stack 142, and the third stack 143. The first stack 141 may include the first hole transport region 1411, the first emission layer 1412, and the first electron transport region 1413. In these embodiments, the first hole transport region 1411 may include the first hole transport layer 1411a and the first auxiliary emission layer 1411b, the first hole transport layer 1411a or the first auxiliary emission layer 1411b may include the first compound represented by Formula 1, the thickness of the first hole transport layer 1411a may be from 250 Å to 700 Å, and 10% to 50% of the thickness of the first hole transport layer 1411a may be doped with the first doping material. In these embodiments, the second stack 142 may include a second hole transport region 1421, a second emission layer 1422, and a second electron transport region 1423. In these embodiments, the second hole transport region 1421 may include a second hole transport layer 1421a and a second auxiliary emission layer 1421b, the second hole transport layer 1421a or the second auxiliary emission layer 1421b may include the second compound represented by Formula 1, the thickness of the second hole transport layer 1421a may be from 250 Å to 700 Å, and 10% to 50% of the thickness of the second hole transport layer 1421a may be doped with the second doping material. In these embodiments, the third stack 143 may include a third hole transport region 1431, a third emission layer 1432, and a third electron transport region 1433. In these embodiments, the third hole transport region 1431 may include a third hole transport layer 1431a and a third auxiliary emission layer 1431b, the third hole transport layer 1431a or the third auxiliary emission layer 1431b may include the third compound represented by Formula 1, the thickness of the third hole transport layer 1431a may be from 250 Å to 700 Å, and 10% to 50% of the thickness of the third hole transport layer 1431a may be doped with the third doping material.

In these embodiments, the thickness of the first hole transport layer 1411a may be from 400 Å to 500 Å, the thickness of the second hole transport layer 1421a may be from 500 Å to 650 Å, and the thickness of the third hole transport layer 1431a may be from 450 Å to 560 Å. For example, when the first electrode 110, the first stack 141, the second stack 142, the third stack 143, and the second electrode 120 are sequentially stacked, each of the first emission layer 1412, the second emission layer 1422, the first hole transport layer 1411a, the second hole transport layer 1421a, and the third hole transport layer 1431a meet the above-described thickness ranges, the third emission layer 1432 includes a blue host and a blue dopant, and the first hole transport layer 1411a or the first auxiliary emission layer 1411b includes the first compound, the second hole transport layer 1421a or the second auxiliary emission layer 1421b includes the second compound, and the third hole transport layer 1431a or the third auxiliary emission layer 1431b includes the third compound, an organic electronic device having superior efficiency or increased lifespan may be provided.

In these embodiments, the first hole transport layer 1411a may include the first doping material-doped layer 1411aa doped with the first doping material and the first doping material undoped layer 1411ab not doped with the first doping material. The first doping material-doped layer 1411aa may include the first compound and 5 to 15 parts by weight of the first doping material with respect to 100 parts by weight of the first compound. The second hole transport layer may include a second doping material-doped layer doped with a second doping material and a second doping material undoped layer not doped with the second doping material. The second doping material-doped layer may include the second compound and 5 to 15 parts by weight of the second doping material with respect to 100 parts by weight of the second compound. The third hole transport layer may include a third doping material-doped layer doped with the third doping material and a third doping material undoped layer not doped with the third doping material. The third doping material-doped layer may include the third compound and 5 to 15 parts by weight of the third doping material with respect to 100 parts by weight of the third compound.

In these embodiments, the first compound, the second compound, and the third compound may be the same compounds.

In some embodiments of the present disclosure, the organic material layer 130 may include the first stack 141, the second stack 142, and the third stack 143. The first stack 141 may include the first hole transport region 1411, the first emission layer 1412, and the first electron transport region 1413. In these embodiments, the first hole transport region 1411 may include the first hole transport layer 1411a and the first auxiliary emission layer 1411b, the first hole transport layer 1411a or the first auxiliary emission layer 1411b may include at least one of the first compound or the fourth compound represented by Formula 1, the thickness of the first hole transport layer 1411a may be from 250 Å to 700 Å, and 10% to 50% of the first hole transport layer 1411a may be doped with the first doping material.

In some embodiments of the present disclosure, the organic material layer 130 may include the first stack 141, the second stack 142, the third stack 143, and the fourth stack 144. The first stack 141 may include the first hole transport region 1411, the first emission layer 1412, and the first electron transport region 1413. In these embodiments, the first hole transport region 1411 may include the first hole transport layer 1411a and the first auxiliary emission layer 1411b, the first hole transport layer 1411a or the first auxiliary emission layer 1411b may include the first compound represented by Formula 1, the thickness of the first hole transport layer 1411a may be from 250 Å to 700 Å, and 10% to 50% of the first hole transport layer 1411a may be doped with the first doping material. In these embodiments, the fourth stack 144 may include the fourth hole transport region 1441, the fourth emission layer 1442, and the fourth electron transport region 1443. In these embodiments, the fourth hole transport region 1441 may include the fourth hole transport layer 1441a and the fourth auxiliary emission layer 1441b, the fourth hole transport layer 1441a or the fourth auxiliary emission layer 1441b may include may include at least one of the fifth compound or the sixth compound represented by Formula 1, the thickness of the fourth hole transport layer 1441a may be from 250 Å to 700 Å, and 10% to 50% of the fourth hole transport layer 1441a may be doped with the fourth doping material.

In embodiments of the present disclosure, at least one of the first emission layer 1412, the second emission layer 1422, or the third emission layer 1432 may be a blue light emission layer. When at least one of the first to third emission layers is a blue light emission layer and the first hole transport layer 1411a or the first auxiliary emission layer 1411b meets the above-described thickness and doping conditions while including the first compound, an organic electronic device superior in terms of efficiency, lifespan, or color purity may be provided.

Herein, the blue light emission layer may refer to an emission layer that emits light having a wavelength ranging from about 450 nm to about 495 nm when excited by electron-hole recombination therein.

In embodiments of the present disclosure, the first emission layer 1412, the second emission layer 1422, and the third emission layer 1432 may emit blue light. When the first to third emission layers are blue light emission layers and the first hole transport layer 1411a or the first auxiliary emission layer 1411b meets the above-described thickness and doping conditions while including the first compound, an organic electronic device superior in efficiency, lifespan, or color purity may be provided.

In embodiments of the present disclosure, one or two of the first emission layer 1412, the second emission layer 1422, and the third emission layer 1432 may be blue light emission layers, and one or two of the first emission layer 1412, the second emission layer 1422, and the third emission layer 1432 may be green light emission layers. When one or two of the first emission layer 1412, the second emission layer 1422, and the third emission layer 1432 are blue light emission layers, one or two of the first emission layer 1412, the second emission layer 1422, and the third emission layer 1432 are green light emission layers, and the first hole transport layer 1411a or the first auxiliary emission layer 1411b meets the above-described thickness and doping conditions while including the first compound, an organic electronic device superior in efficiency, lifespan, or color purity may be provided.

Herein, the green light emission layers may refer to emission layers each of which emits light having a wavelength ranging from about 495 nm to about 570 nm when excited by electron-hole recombination therein.

In embodiments of the present disclosure, two emission layers of the first emission layer 1412, the second emission layer 1422, and the third emission layer 1432 may be blue light emission layers, and the remaining one emission layer of the first emission layer 1412, the second emission layer 1422, and the third emission layer 1432 may be a green light emission layer. When two emission layers of the first emission layer 1412, the second emission layer 1422, and the third emission layer 1432 are blue light emission layers, the remaining one emission layer of the first emission layer 1412, the second emission layer 1422, and the third emission layer 1432 is a green light emission layer, and the first hole transport layer 1411a or the first auxiliary emission layer 1411b meets the above-described thickness and doping conditions while including the first compound, an organic electronic device superior in efficiency, lifespan, or color purity may be provided.

In embodiments of the present disclosure, when two emission layers of the first emission layer 1412, the second emission layer 1422, and the third emission layer 1432 are blue light emission layers and the remaining emission layer is a green light emission layer, the green light emission layer may be positioned between the two blue light emission layers. When the first hole transport layer 1411a or the first auxiliary emission layer 1411b includes the first compound and meets the above-described thickness and doping conditions while the first to third emission layers meet the above-described conditions, an organic electronic device superior in efficiency, lifespan, or color purity may be provided.

In embodiments of the present disclosure, at least one of the first emission layer 1412, the second emission layer 1422, or the third emission layer 1432 may be a multi-emission layer emitting green light and blue light.

Herein, the multi-emission layer emitting green light and blue light may refer to an emission layer emitting light having a wavelength ranging from about 450 nm to about 570 nm when excited by electron-hole recombination therein.

When at least one of the first emission layer 1412, the second emission layer 1422, or the third emission layer 1432 is a multi-emission layer emitting green light and blue light and the first hole transport layer 1411a or the first auxiliary emission layer 1411b meets the above-described thickness and doping conditions while including the first compound, an organic electronic device superior in efficiency, lifespan, or color purity may be provided.

In embodiments of the present disclosure, two emission layers of the first emission layer 1412 to the fourth emission layer 1442 may be blue light emission layers, whereas the remaining one emission layer different from the two emission layers may be a green light emission layer. When two emission layers of the first emission layer 1412 to the fourth emission layer 1442 are blue light emission layers, the remaining one emission layer different from the two emission layers is a green light emission layer, and the fourth hole transport layer 1441a or the fourth auxiliary emission layer 1441b meets the above-described thickness and doping conditions while including at least one of the fifth compound or the sixth compound, an organic electronic device superior in efficiency, lifespan, or color purity may be provided.

In embodiments of the present disclosure, three light emission layers of the first emission layer 1412 to the fourth emission layer 1442 may be blue light emission layers, and the remaining emission layer may be a light emission layer. When the three light emission layers of the first emission layer 1412 to the fourth emission layer 1442 are blue light emission layers, the remaining emission layer is a light emission layer, and the fourth hole transport layer 1441a or the fourth auxiliary emission layer 1441b meets the above-described thickness and doping conditions while including at least one of the fifth compound or the sixth compound, an organic electronic device superior in efficiency, lifespan, or color purity may be provided.

Hereinafter, the present disclosure will be described in detail with reference to, but is not limited to, examples of synthesis of the compound of the hole transport layer and examples of preparation of the organic electronic device.

### SYNTHESIS EXAMPLES

A compound (e.g., the fourth compound, the sixth compound, the seventh compound, or the eighth compound described above) represented by Formula C and including a radical of a compound represented by Formula A or Formula B, according to the present disclosure, is prepared by, but is not limited to, reacting one of Sub 1-A to Sub 1-C with Sub 2 as in the following Reaction Formula 1.

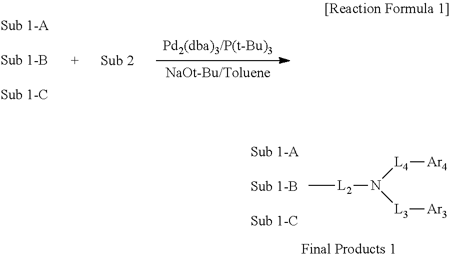

More specifically, Reaction Formula 1 may be represented by, but is not limited to, the following Reaction Formula 2 to Reaction Formula 4.

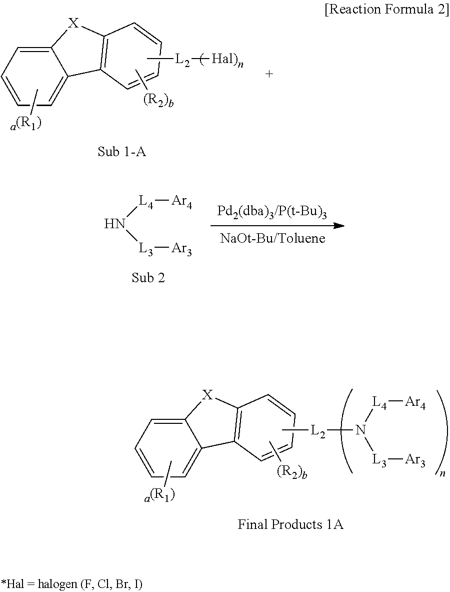

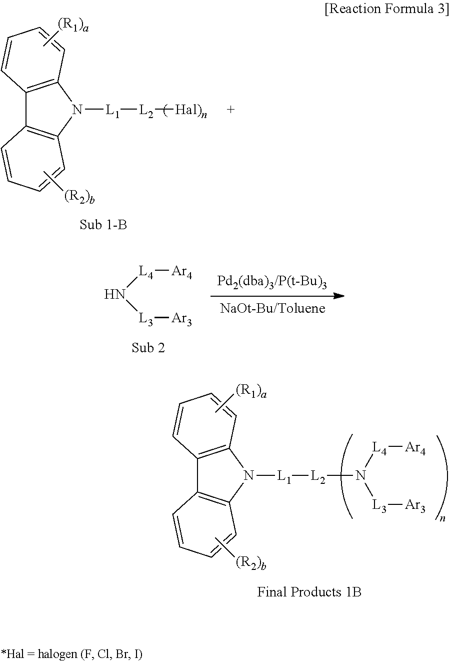

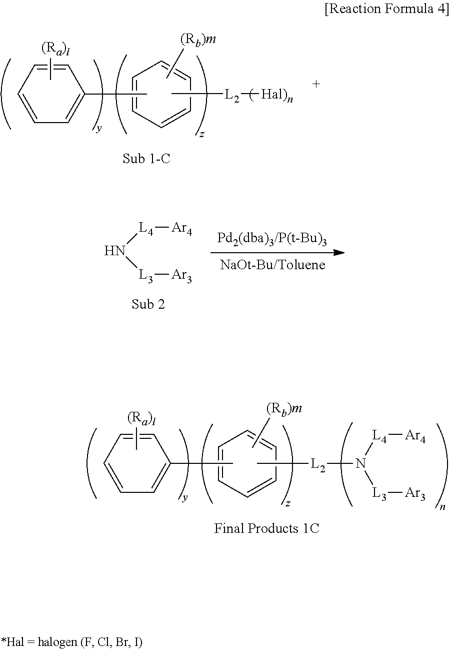

Synthesis of Sub 1-A

Sub 1A of Reaction Formula 1 may be synthesized through, but is not limited to, a reaction path of the following Reaction Formula 5.

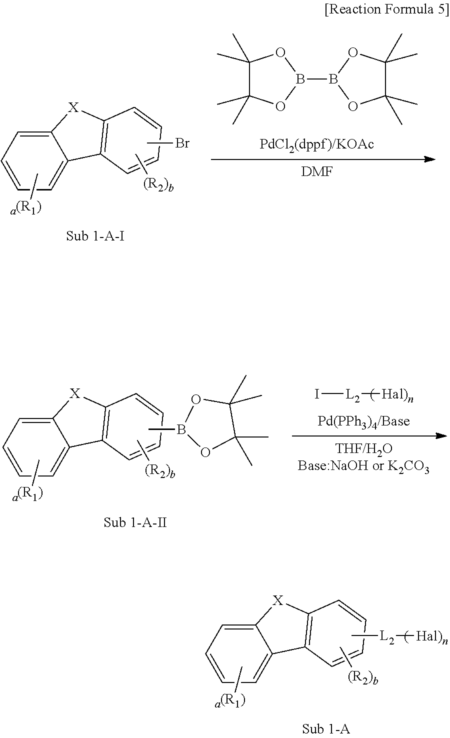

Synthesis of Sub 1-B

Sub 1B of Reaction Formula 2 may be synthesized through, but is not limited to, a reaction path of the following Reaction Formula 6.

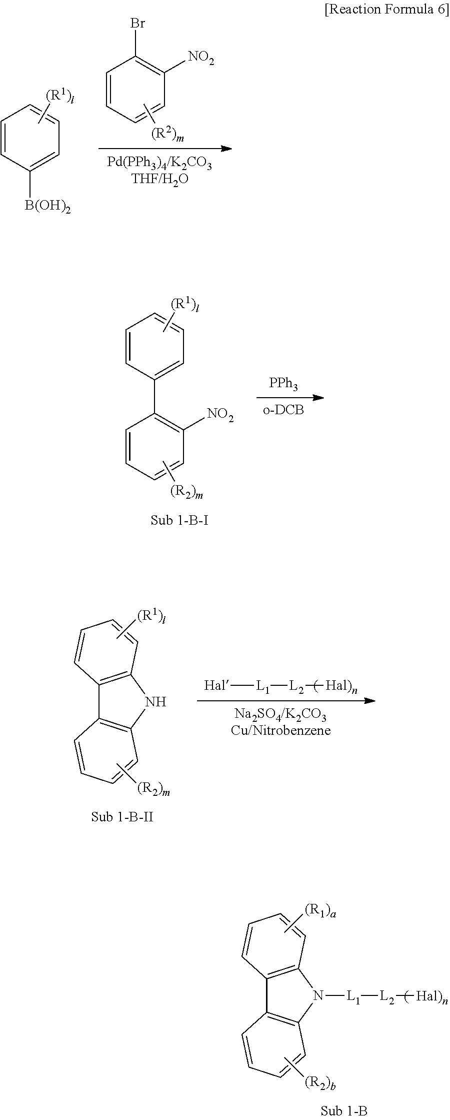

Synthesis of Sub 1-C

Sub 1C of Reaction Formula 3 may be synthesized through, but is not limited to, a reaction path of the following Reaction Formula 7.

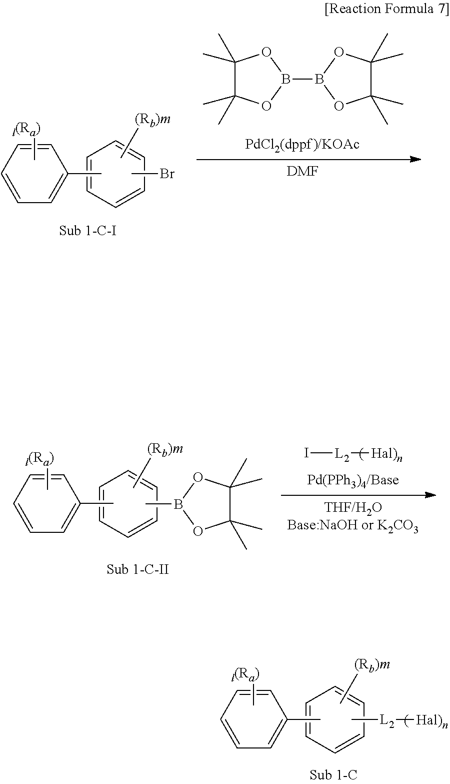

Illustration of Synthesis of Sub 1-A-3

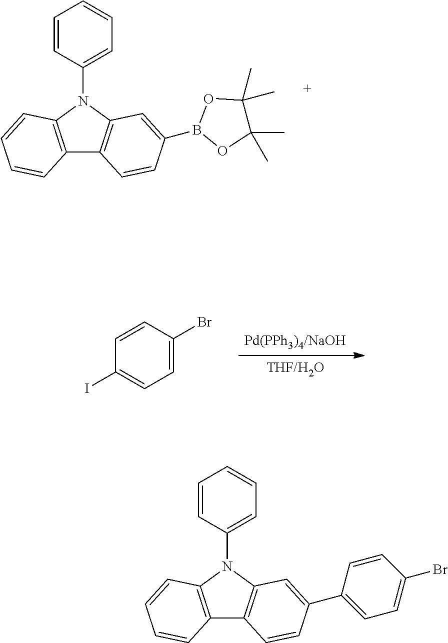 [SMILES: CC1(C)OB(c2ccc3c4ccccc4n(-c4ccccc4)c3c2)OC1(C)C ; Brc1ccc(I)cc1 ; "Pd(PPh3)4/NaOH" ; "THF/H2O" ; Brc1ccc(-c2ccc3c4ccccc4n(-c4ccccc4)c3c2)cc1]

9-phenyl-2-(4,4,5,5-tetramethyl-1,3,2-dioxaborolan-2-yl)-9H-carbazole (29.5 g, 80 mmol), THF 360 ml, 1-bromo-4-iodobenzene (23.8 g, 84 mmol), Pd(PPh₃)₄ (2.8 g, 2.4 mmol), NaOH (9.6 g, 240 mmol), and water 180 ml were added, followed by stirring and refluxing. When the reaction was completed, extraction was performed with ether and water, and then an organic layer was dried with MgSO₄ and concentrated. The resultant organic matter was subjected to silica gel column chromatography and recrystallization to create a product in an amount of 22.9 g (72%).

Illustration of Synthesis of Sub 1-A-5

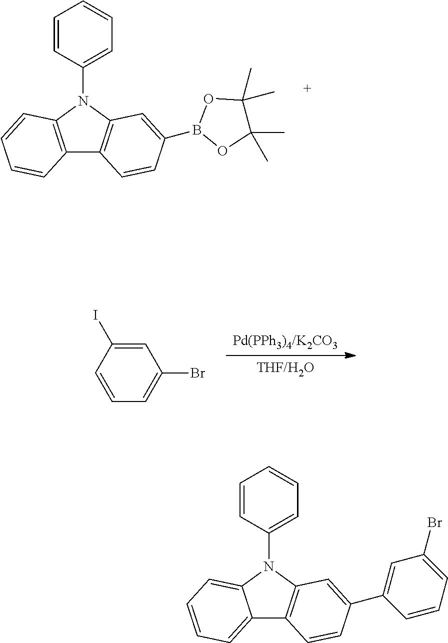 [SMILES: CC1(C)OB(c2ccc3c4ccccc4n(-c4ccccc4)c3c2)OC1(C)C ; Brc1cccc(I)c1 ; "Pd(PPh3)4/K2CO3" ; "THF/H2O" ; Brc1cccc(-c2ccc3c4ccccc4n(-c4ccccc4)c3c2)c1]

After 9-phenyl-2-(4,4,5,5-tetramethyl-1,3,2-dioxaborolan-2-yl)-9H-carbazole (73.92 g, 200.2 mmol) was dissolved with THF 880 ml in a round bottom flask, 1-bromo-2-iodobenzene (85.0 g, 300.3 mmol), Pd(PPh₃)₄ (11.6 g, 10 mmol), K₂CO₃ (83 g, 600.6 mmol), and water 440 ml were processed using the same experimental method as in Sub 1-A-3, thereby creating a product in an amount of 55.8 g (yield: 70%).

Sub 1-B-2 Synthesis Example

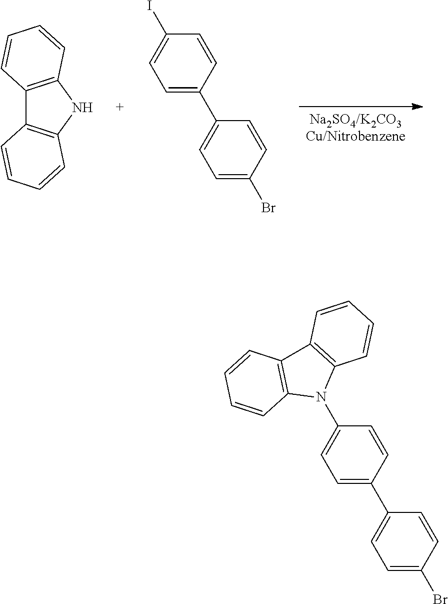 [SMILES: c1ccc2c(c1)[nH]c1ccccc12 ; Brc1ccc(-c2ccc(I)cc2)cc1 ; "Na2SO4/K2CO3Cu/Nitrobenzene" ; Brc1ccc(-c2ccc(-n3c4ccccc4c4ccccc43)cc2)cc1]

After 9H-carbazole (12 g, 71.8 mmol) was dissolved with nitrobenzene (450 ml) in a round bottom flask, 4-bromo-4′-iodo-1,1′-biphenyl (38.65 g, 107.6 mmol), Na₂SO₄ (10.19 g, 71.8 mmol), K₂CO₃ (9.92 g, 71.8 mmol), and Cu (1.37 g, 21.5 mmol) were added, followed by stirring at 200° C. When the reaction was completed, nitrobenzene was removed by distillation, extraction was performed with CH₂Cl₂ and water, and an organic layer was dried with MgSO₄ and concentrated. Afterwards, a resultant compound was subjected to silica gel column chromatography and recrystallization, thereby creating a product 21.15 g (yield: 74%).

TABLE 1

| | |
|---|---|
| 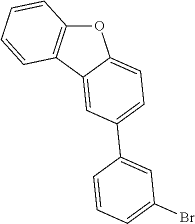 [SMILES: Brc1cccc(-c2ccc3oc4ccccc4c3c2)c1] | Sub 1-A-53 |
| 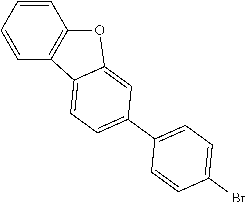 [SMILES: Brc1ccc(-c2ccc3c(c2)oc2ccccc23)cc1] | Sub 1-A-54 |
| 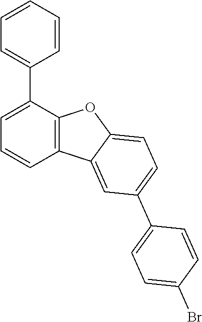 [SMILES: Brc1ccc(-c2ccc3oc4c(-c5ccccc5)cccc4c3c2)cc1] | Sub 1-A-55 |
| 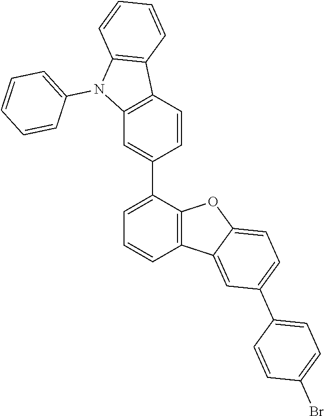 [SMILES: Brc1ccc(-c2ccc3oc4c(-c5ccc6c7ccccc7n(-c7ccccc7)c6c5)cccc4c3c2)cc1] | Sub 1-A-56 |
| 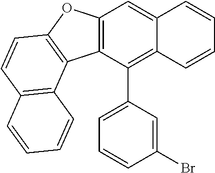 [SMILES: Brc1cccc(-c2c3ccccc3cc3oc4ccc5ccccc5c4c23)c1] | Sub 1-A-57 |
| 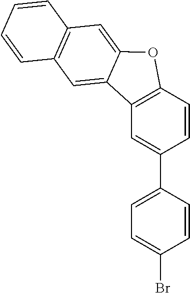 [SMILES: Brc1ccc(-c2ccc3oc4cc5ccccc5cc4c3c2)cc1] | Sub 1-A-58 |
| 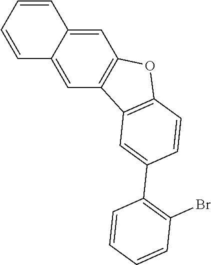 [SMILES: Brc1ccccc1-c1ccc2oc3cc4ccccc4cc3c2c1] | Sub 1-A-59 |
| 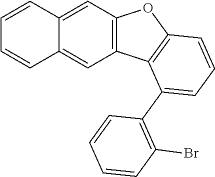 [SMILES: Brc1ccccc1-c1cccc2oc3cc4ccccc4cc3c12] | Sub 1-A-60 |
| 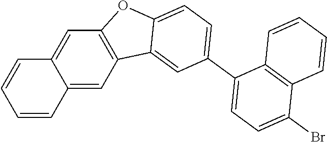 [SMILES: Brc1ccc(-c2ccc3oc4cc5ccccc5cc4c3c2)c2ccccc12] | Sub 1-A-61 |
| 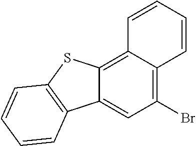 [SMILES: Brc1cc2c3ccccc3sc2c2ccccc12] | Sub 1-A-62 |
| 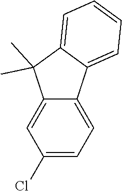 [SMILES: CC1(C)c2ccccc2-c2ccc(Cl)cc21] | Sub 1-A-63 |
| 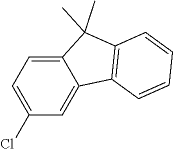 [SMILES: CC1(C)c2ccccc2-c2cc(Cl)ccc21] | Sub 1-A-64 |
| 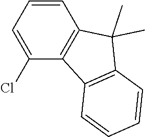 [SMILES: CC1(C)c2ccccc2-c2c(Cl)cccc21] | Sub 1-A-65 |
| 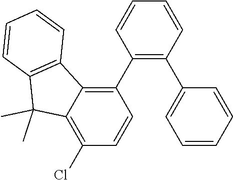 [SMILES: CC1(C)c2ccccc2-c2c(-c3ccccc3-c3ccccc3)ccc(Cl)c21] | Sub 1-A-66 |
| 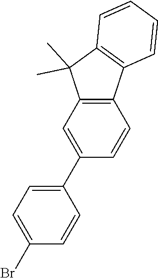 [SMILES: CC1(C)c2ccccc2-c2ccc(-c3ccc(Br)cc3)cc21] | Sub 1-A-67 |
| 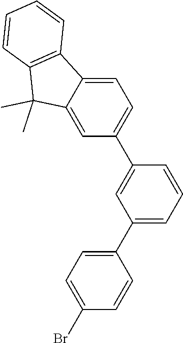 [SMILES: CC1(C)c2ccccc2-c2ccc(-c3cccc(-c4ccc(Br)cc4)c3)cc21] | Sub 1-A-68 |
| 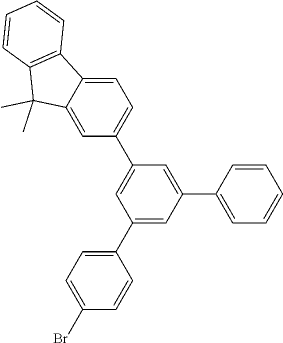 [SMILES: CC1(C)c2ccccc2-c2ccc(-c3cc(-c4ccccc4)cc(-c4ccc(Br)cc4)c3)cc21] | Sub 1-A-69 |
| 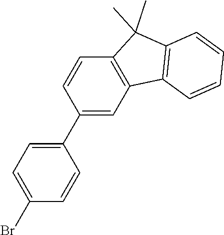 [SMILES: CC1(C)c2ccccc2-c2cc(-c3ccc(Br)cc3)ccc21] | Sub 1-A-70 |
| 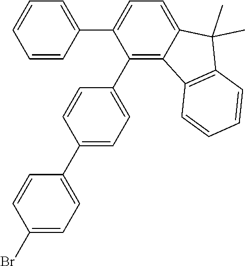 [SMILES: CC1(C)c2ccccc2-c2c1ccc(-c1ccccc1)c2-c1ccc(-c2ccc(Br)cc2)cc1] | Sub 1-A-71 |
| 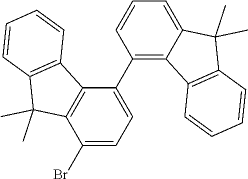 | Sub 1-A-72 |
| 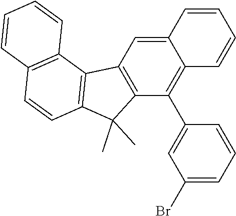 [SMILES: CC1(C)c2ccc3ccccc3c2-c2cc3ccccc3c(-c3cccc(Br)c3)c21] | Sub 1-A-73 |
| 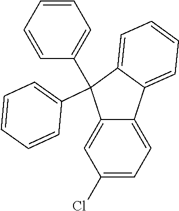 [SMILES: Clc1ccc2c(c1)C(c1ccccc1)(c1ccccc1)c1ccccc1-2] | Sub 1-A-74 |
| 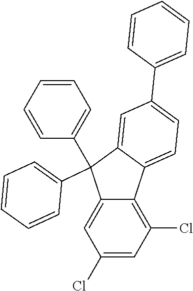 [SMILES: Clc1cc(Cl)c2c(c1)C(c1ccccc1)(c1ccccc1)c1cc(-c3ccccc3)ccc1-2] | Sub 1-A-75 |
| 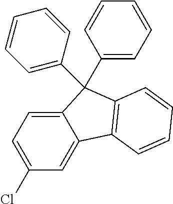 [SMILES: Clc1ccc2c(c1)-c1ccccc1C2(c1ccccc1)c1ccccc1] | Sub 1-A-76 |
| 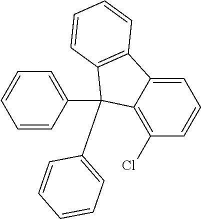 [SMILES: Clc1cccc2c1C(c1ccccc1)(c1ccccc1)c1ccccc1-2] | Sub 1-A-77 |
| 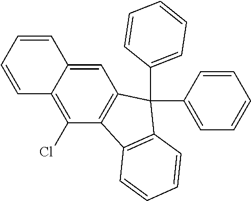 [SMILES: Clc1c2c(cc3ccccc13)C(c1ccccc1)(c1ccccc1)c1ccccc1-2] | Sub 1-A-78 |
| 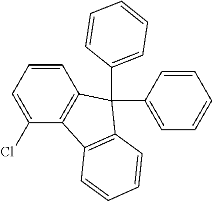 [SMILES: Clc1cccc2c1-c1ccccc1C2(c1ccccc1)c1ccccc1] | Sub 1-A-79 |
| 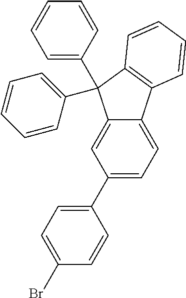 [SMILES: Brc1ccc(-c2ccc3c(c2)C(c2ccccc2)(c2ccccc2)c2ccccc2-3)cc1] | Sub 1-A-80 |
| 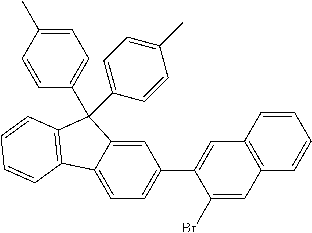 [SMILES: Cc1ccc(C2(c3ccc(C)cc3)c3ccccc3-c3ccc(-c4cc5ccccc5cc4Br)cc32)cc1] | Sub 1-A-81 |
| 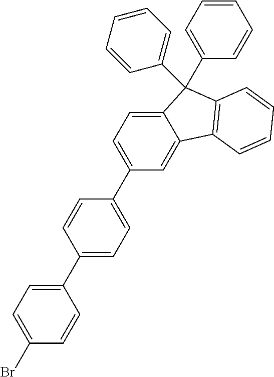 [SMILES: Brc1ccc(-c2ccc(-c3ccc4c(c3)-c3ccccc3C4(c3ccccc3)c3ccccc3)cc2)cc1] | Sub 1-A-82 |
| 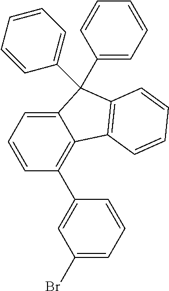 [SMILES: Brc1cccc(-c2cccc3c2-c2ccccc2C3(c2ccccc2)c2ccccc2)c1] | Sub 1-A-83 |
| 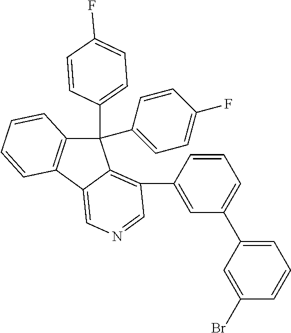 [SMILES: Fc1ccc(C2(c3ccc(F)cc3)c3ccccc3-c3cncc(-c4cccc(-c5cccc(Br)c5)c4)c32)cc1] | Sub 1-A-84 |
| 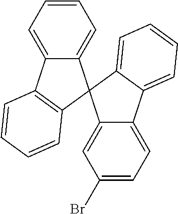 [SMILES: Brc1ccc2c(c1)C1(c3ccccc3-c3ccccc31)c1ccccc1-2] | Sub 1-A-85 |
| 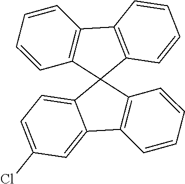 [SMILES: Clc1ccc2c(c1)-c1ccccc1C21c2ccccc2-c2ccccc21] | Sub 1-A-86 |
| 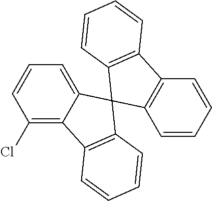 [SMILES: Clc1cccc2c1-c1ccccc1C21c2ccccc2-c2ccccc21] | Sub 1-A-87 |
| 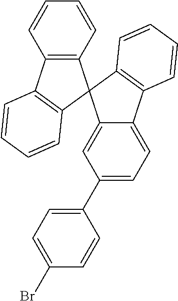 [SMILES: Brc1ccc(-c2ccc3c(c2)C2(c4ccccc4-c4ccccc42)c2ccccc2-3)cc1] | Sub 1-A-88 |
| 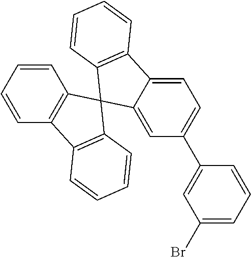 [SMILES: Brc1cccc(-c2ccc3c(c2)C2(c4ccccc4-c4ccccc42)c2ccccc2-3)c1] | Sub 1-A-89 |
| 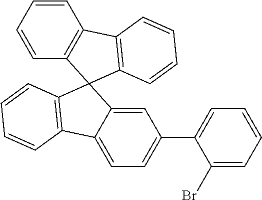 [SMILES: Brc1ccccc1-c1ccc2c(c1)C1(c3ccccc3-c3ccccc31)c1ccccc1-2] | Sub 1-A-90 |
| 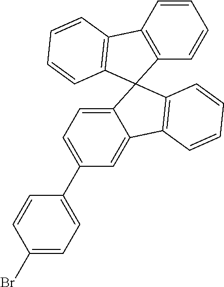 [SMILES: Brc1ccc(-c2ccc3c(c2)-c2ccccc2C32c3ccccc3-c3ccccc32)cc1] | Sub 1-A-91 |
| 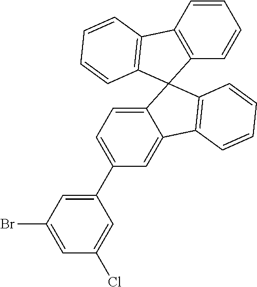 [SMILES: Clc1cc(Br)cc(-c2ccc3c(c2)-c2ccccc2C32c3ccccc3-c3ccccc32)c1] | Sub 1-A-92 |
| 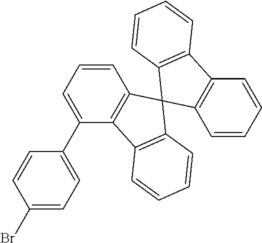 [SMILES: Brc1ccc(-c2cccc3c2-c2ccccc2C32c3ccccc3-c3ccccc32)cc1] | Sub 1-A-93 |
| 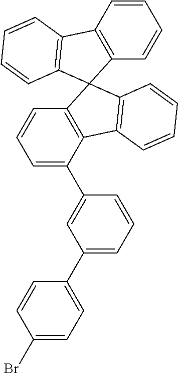 [SMILES: Brc1ccc(-c2cccc(-c3cccc4c3-c3ccccc3C43c4ccccc4-c4ccccc43)c2)cc1] | Sub 1-A-94 |
| 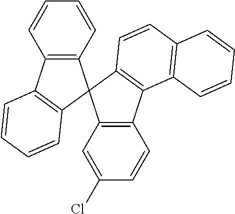 [SMILES: Clc1ccc2c(c1)C1(c3ccccc3-c3ccccc31)c1ccc3ccccc3c1-2] | Sub 1-A-95 |
| 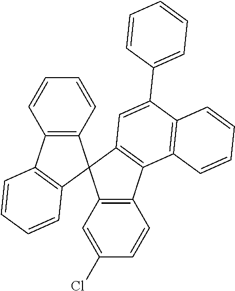 [SMILES: Clc1ccc2c(c1)C1(c3ccccc3-c3ccccc31)c1cc(-c3ccccc3)c3ccccc3c1-2] | Sub 1-A-96 |
| 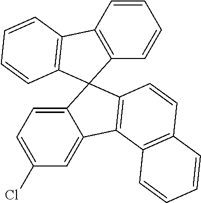 [SMILES: Clc1ccc2c(c1)-c1c(ccc3ccccc13)C21c2ccccc2-c2ccccc21] | Sub 1-A-97 |
| 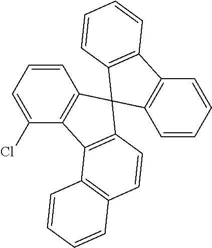 [SMILES: Clc1cccc2c1-c1c(ccc3ccccc13)C21c2ccccc2-c2ccccc21] | Sub 1-A-98 |
| 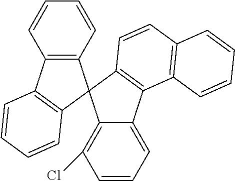 [SMILES: Clc1cccc2c1C1(c3ccccc3-c3ccccc31)c1ccc3ccccc3c1-2] | Sub 1-A-99 |
| 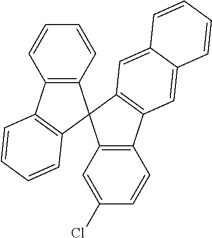 [SMILES: Clc1ccc2c(c1)C1(c3ccccc3-c3ccccc31)c1cc3ccccc3cc1-2] | Sub 1-A-100 |
| 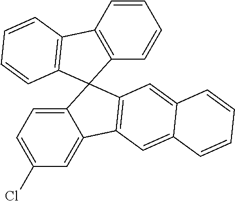 [SMILES: Clc1ccc2c(c1)-c1cc3ccccc3cc1C21c2ccccc2-c2ccccc21] | Sub 1-A-101 |
| 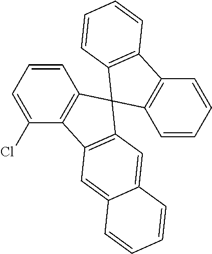 [SMILES: Clc1cccc2c1-c1cc3ccccc3cc1C21c2ccccc2-c2ccccc21] | Sub 1-A-102 |
| 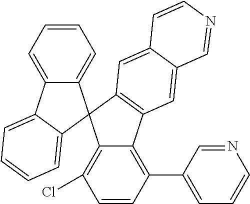 [SMILES: Clc1ccc(-c2cccnc2)c2c1C1(c3ccccc3-c3ccccc31)c1cc3ccncc3cc1-2] | Sub 1-A-103 |
| 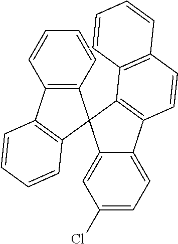 [SMILES: Clc1ccc2c(c1)C1(c3ccccc3-c3ccccc31)c1c-2ccc2ccccc12] | Sub 1-A-104 |
| 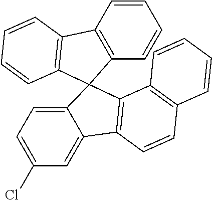 [SMILES: Clc1ccc2c(c1)-c1ccc3ccccc3c1C21c2ccccc2-c2ccccc21] | Sub 1-A-105 |
| 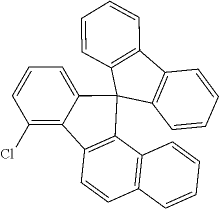 [SMILES: Clc1cccc2c1-c1ccc3ccccc3c1C21c2ccccc2-c2ccccc21] | Sub 1-A-106 |
| 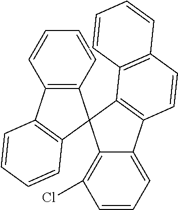 [SMILES: Clc1cccc2c1C1(c3ccccc3-c3ccccc31)c1c-2ccc2ccccc12] | Sub 1-A-107 |
| 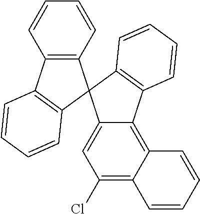 [SMILES: Clc1cc2c(c3ccccc13)-c1ccccc1C21c2ccccc2-c2ccccc21] | Sub 1-A-108 |
| 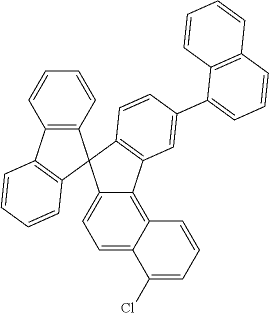 [SMILES: Clc1cccc2c3c(ccc12)C1(c2ccccc2-c2ccccc21)c1ccc(-c2cccc4ccccc24)cc1-3] | Sub 1-A-109 |
| 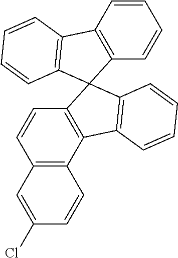 [SMILES: Clc1ccc2c3c(ccc2c1)C1(c2ccccc2-c2ccccc21)c1ccccc1-3] | Sub 1-A-110 |
| 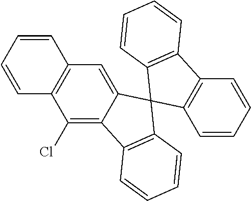 [SMILES: Clc1c2c(cc3ccccc13)C1(c3ccccc3-c3ccccc31)c1ccccc1-2] | Sub 1-A-111 |
| 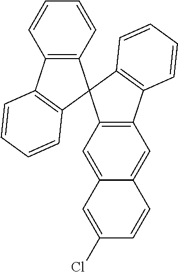 [SMILES: Clc1ccc2cc3c(cc2c1)C1(c2ccccc2-c2ccccc21)c1ccccc1-3] | Sub 1-A-112 |
| 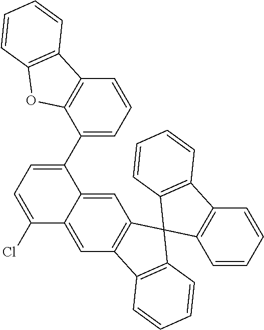 [SMILES: Clc1ccc(-c2cccc3c2oc2ccccc23)c2cc3c(cc12)-c1ccccc1C31c2ccccc2-c2ccccc21] | Sub 1-A-113 |
| 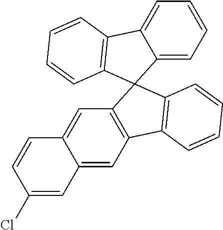 [SMILES: Clc1ccc2cc3c(cc2c1)-c1ccccc1C31c2ccccc2-c2ccccc21] | Sub 1-A-114 |
| 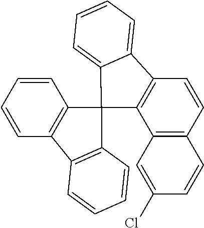 [SMILES: Clc1ccc2ccc3c(c2c1)C1(c2ccccc2-c2ccccc21)c1ccccc1-3] | Sub 1-A-115 |
| 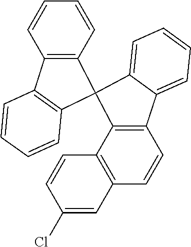 [SMILES: Clc1ccc2c3c(ccc2c1)-c1ccccc1C31c2ccccc2-c2ccccc21] | Sub 1-A-116 |
| 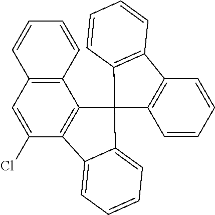 [SMILES: Clc1cc2ccccc2c2c1-c1ccccc1C21c2ccccc2-c2ccccc21] | Sub 1-A-117 |
| 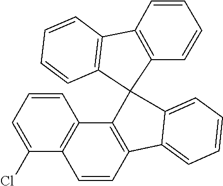 [SMILES: Clc1cccc2c3c(ccc12)-c1ccccc1C31c2ccccc2-c2ccccc21] | Sub 1-A-118 |
| 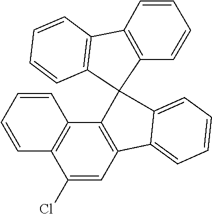 [SMILES: Clc1cc2c(c3ccccc13)C1(c3ccccc3-c3ccccc31)c1ccccc1-2] | Sub 1-A-119 |
| 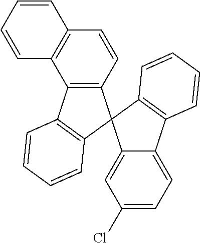 [SMILES: Clc1ccc2c(c1)C1(c3ccccc3-2)c2ccccc2-c2c1ccc1ccccc21] | Sub 1-A-120 |
| 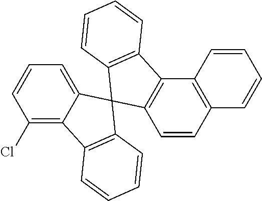 [SMILES: Clc1cccc2c1-c1ccccc1C21c2ccccc2-c2c1ccc1ccccc21] | Sub 1-A-121 |
| 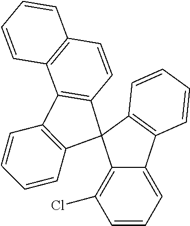 [SMILES: Clc1cccc2c1C1(c3ccccc3-2)c2ccccc2-c2c1ccc1ccccc21] | Sub 1-A-122 |
| 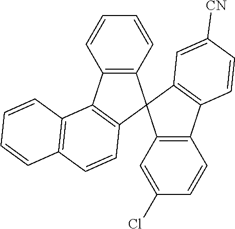 [SMILES: N#Cc1ccc2c(c1)C1(c3cc(Cl)ccc3-2)c2ccccc2-c2c1ccc1ccccc21] | Sub 1-A-123 |
| 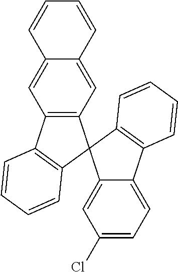 [SMILES: Clc1ccc2c(c1)C1(c3ccccc3-2)c2ccccc2-c2cc3ccccc3cc21] | Sub 1-A-124 |
| 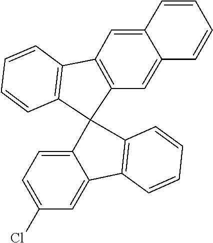 [SMILES: Clc1ccc2c(c1)-c1ccccc1C21c2ccccc2-c2cc3ccccc3cc21] | Sub 1-A-125 |
| 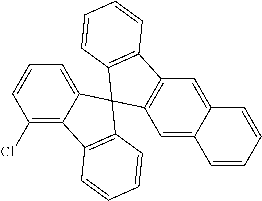 [SMILES: Clc1cccc2c1-c1ccccc1C21c2ccccc2-c2cc3ccccc3cc21] | Sub 1-A-126 |
| 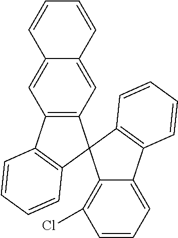 [SMILES: Clc1cccc2c1C1(c3ccccc3-c3cc4ccccc4cc31)c1ccccc1-2] | Sub 1-A-127 |
| 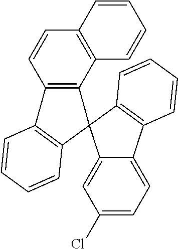 [SMILES: Clc1ccc2c(c1)C1(c3ccccc3-2)c2ccccc2-c2ccc3ccccc3c21] | Sub 1-A-128 |
| 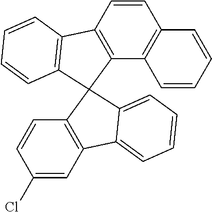 [SMILES: Clc1ccc2c(c1)-c1ccccc1C21c2ccccc2-c2ccc3ccccc3c21] | Sub 1-A-129 |
| 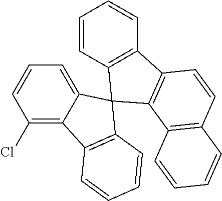 [SMILES: Clc1cccc2c1-c1ccccc1C21c2ccccc2-c2ccc3ccccc3c21] | Sub 1-A-130 |
| 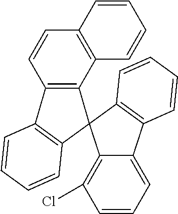 [SMILES: Clc1cccc2c1C1(c3ccccc3-2)c2ccccc2-c2ccc3ccccc3c21] | Sub 1-A-131 |
| 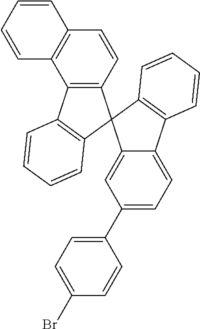 [SMILES: Brc1ccc(-c2ccc3c(c2)C2(c4ccccc4-3)c3ccccc3-c3c2ccc2ccccc32)cc1] | Sub 1-A-132 |
| 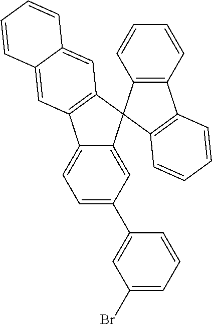 [SMILES: Brc1cccc(-c2ccc3c(c2)C2(c4ccccc4-c4ccccc42)c2cc4ccccc4cc2-3)c1] | Sub 1-A-133 |
| 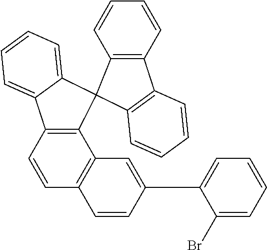 [SMILES: Brc1ccccc1-c1ccc2ccc3c(c2c1)C1(c2ccccc2-c2ccccc21)c1ccccc1-3] | Sub 1-A-134 |
| 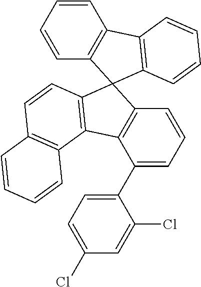 [SMILES: Clc1ccc(-c2cccc3c2-c2c(ccc4ccccc24)C32c3ccccc3-c3ccccc32)c(Cl)c1] | Sub 1-A-135 |
| 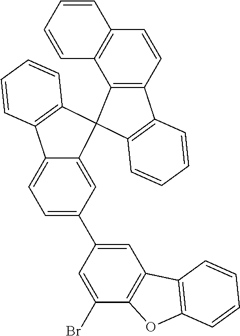 [SMILES: Brc1cc(-c2ccc3c(c2)C2(c4ccccc4-3)c3ccccc3-c3ccc4ccccc4c32)cc2c1oc1ccccc12] | Sub 1-A-136 |
| 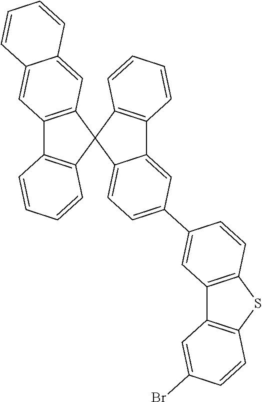 [SMILES: Brc1ccc2sc3ccc(-c4ccc5c(c4)-c4ccccc4C54c5ccccc5-c5cc6ccccc6cc54)cc3c2c1] | Sub 1-A-137 |
| 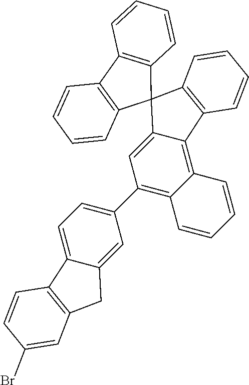 [SMILES: Brc1ccc2c(c1)Cc1cc(-c3cc4c(c5ccccc35)-c3ccccc3C43c4ccccc4-c4ccccc43)ccc1-2] | Sub 1-A-138 |
| 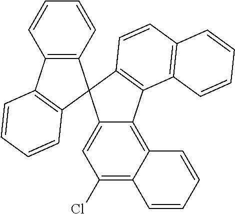 [SMILES: Clc1cc2c(c3ccccc13)-c1c(ccc3ccccc13)C21c2ccccc2-c2ccccc21] | Sub 1-A-139 |
| 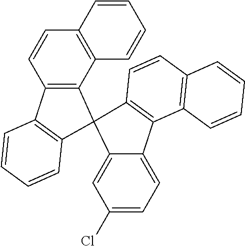 [SMILES: Clc1ccc2c(c1)C1(c3ccccc3-c3ccc4ccccc4c31)c1ccc3ccccc3c1-2] | Sub 1-A-140 |
| 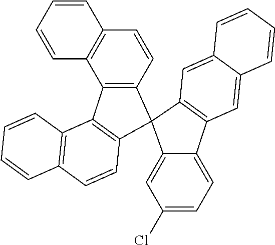 [SMILES: Clc1ccc2c(c1)C1(c3cc4ccccc4cc3-2)c2ccc3ccccc3c2-c2c1ccc1ccccc21] | Sub 1-A-141 |
| 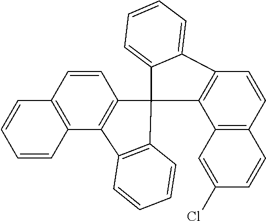 [SMILES: Clc1ccc2ccc3c(c2c1)C1(c2ccccc2-3)c2ccccc2-c2c1ccc1ccccc21] | Sub 1-A-142 |
| 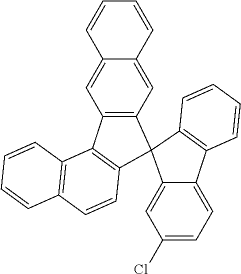 [SMILES: Clc1ccc2c(c1)C1(c3ccccc3-2)c2cc3ccccc3cc2-c2c1ccc1ccccc21] | Sub 1-A-143 |
| 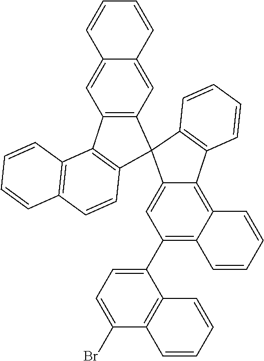 [SMILES: Brc1ccc(-c2cc3c(c4ccccc24)-c2ccccc2C32c3cc4ccccc4cc3-c3c2ccc2ccccc32)c2ccccc12] | Sub 1-A-144 |
| 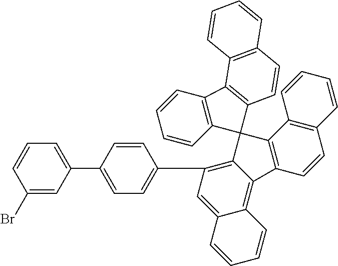 [SMILES: Brc1cccc(-c2ccc(-c3cc4ccccc4c4c3C3(c5ccccc5-c5c3ccc3ccccc53)c3c-4ccc4ccccc34)cc2)c1] | Sub 1-A-145 |
| 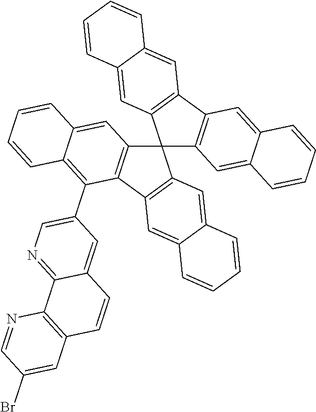 [SMILES: Brc1cnc2c(ccc3cc(-c4c5c(cc6ccccc46)C4(c6cc7ccccc7cc6-c6cc7ccccc7cc64)c4cc6ccccc6cc4-5)cnc32)c1] | Sub 1-A-146 |
| 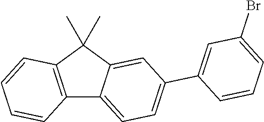 [SMILES: CC1(C)c2ccccc2-c2ccc(-c3cccc(Br)c3)cc21] | Sub 1-A-147 |
| 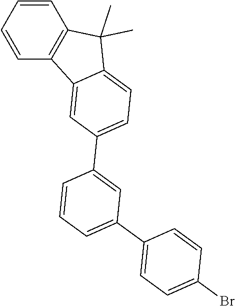 [SMILES: CC1(C)c2ccccc2-c2cc(-c3cccc(-c4ccc(Br)cc4)c3)ccc21] | Sub 1-A-148 |
| 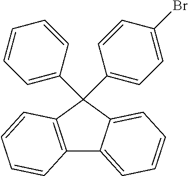 [SMILES: Brc1ccc(C2(c3ccccc3)c3ccccc3-c3ccccc32)cc1] | Sub 1-A-149 |
| 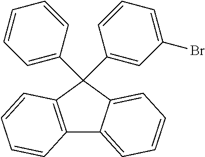 [SMILES: Brc1cccc(C2(c3ccccc3)c3ccccc3-c3ccccc32)c1] | Sub 1-A-150 |
| 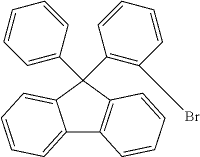 [SMILES: Brc1ccccc1C1(c2ccccc2)c2ccccc2-c2ccccc21] | Sub 1-A-151 |
| 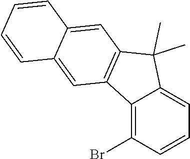 [SMILES: CC1(C)c2cc3ccccc3cc2-c2c(Br)cccc21] | Sub 1-A-152 |
| 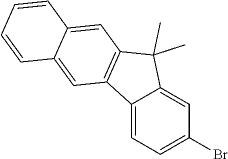 [SMILES: CC1(C)c2cc(Br)ccc2-c2cc3ccccc3cc21] | Sub 1-A-153 |
| 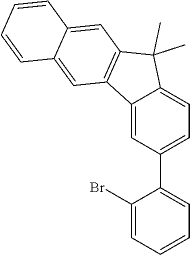 [SMILES: CC1(C)c2ccc(-c3ccccc3Br)cc2-c2cc3ccccc3cc21] | Sub 1-A-154 |
| 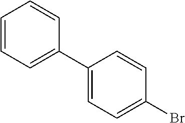 [SMILES: Brc1ccc(-c2ccccc2)cc1] | Sub 1-A-155 |
| 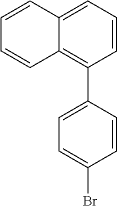 [SMILES: Brc1ccc(-c2cccc3ccccc23)cc1] | Sub 1-A-156 |
| 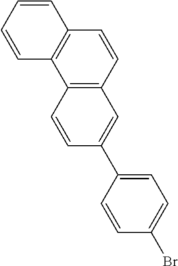 [SMILES: Brc1ccc(-c2ccc3c(ccc4ccccc43)c2)cc1] | Sub 1-A-157 |
| 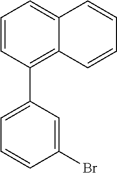 [SMILES: Brc1cccc(-c2cccc3ccccc23)c1] | Sub 1-A-158 |
| 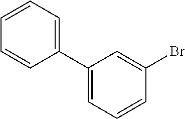 [SMILES: Brc1cccc(-c2ccccc2)c1] | Sub 1-A-159 |
| 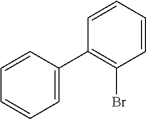 [SMILES: Brc1ccccc1-c1ccccc1] | Sub 1-A-160 |
| 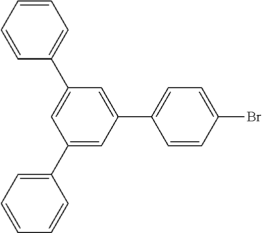 [SMILES: Brc1ccc(-c2cc(-c3ccccc3)cc(-c3ccccc3)c2)cc1] | Sub 1-A-161 |
| 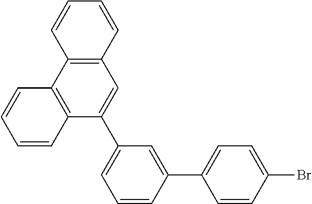 [SMILES: Brc1ccc(-c2cccc(-c3cc4ccccc4c4ccccc34)c2)cc1] | Sub 1-A-162 |

| Compound | FD-MS |
|---|---|
| Sub 1-A-1 | m/z = 321.02 (C₁₈H₁₂BrN = 322.21) |
| Sub 1-A-2 | m/z = 321.02 (C₁₈H₁₂BrN = 322.21) |
| Sub 1-A-3 | m/z = 397.05 (C₂₄H₁₆BrN = 398.30) |
| Sub 1-A-4 | m/z = 563.12 (C₃₇H₂₆BrN = 564.53) |
| Sub 1-A-5 | m/z = 397.05 (C₂₄H₁₆BrN = 398.30) |
| Sub 1-A-6 | m/z = 397.05 (C₂₄H₂₆BrN = 398.30) |
| Sub 1-A-7 | m/z = 473.08 (C [symbol] H [symbol] BrN = 474.40) |
| Sub 1-A-8 | m/z = 473.08 (C [symbol] H [symbol] BrN = 474.40) |
| Sub 1-A-9 | m/z = 473.08 (C₃₀H₂₀BrN = 474.40) |
| Sub 1-A-10 | m/z = 473.08 (C₃₀H₂₀BrN = 474.40) |
| Sub 1-A-11 | m/z = 473.08 (C₃₀H₂₀BrN = 474.40) |
| Sub 1-A-12 | m/z = 473.08 (C₃₀H₂₀BrN = 474.40) |
| Sub 1-A-13 | m/z = 497.08 (C₃₂H₂₀BrN = 498.42) |
| Sub 1-A-14 | m/z = 503.03 (C₃₀H₁₈BrNS = 504.45) |
| Sub 1-A-15 | m/z = 487.06 (C [symbol] H₁₈BrNO = 488.38) |
| Sub 1-A-16 | m/z = 513.11 (C₃₃H₂₄BrN = 514.47) |
| Sub 1-A-17 | m/z = 473.08 (C₃₀H₂₈BrN = 474.40) |
| Sub 1-A-18 | m/z = 628.13 (C [symbol] H₂₅BrN₄ = 629.56) |
| Sub 1-A-19 | m/z = 589.14 (C [symbol] H₂₈BrN = 590.56) |
| Sub 1-A-20 | m/z = 627.13 (C₄₀H₂₆BrN₃ = 628.57) |
| Sub 1-A-21 | m/z = 473.08 (C [symbol] H₂₉BrN = 474.40) |
| Sub 1-A-22 | m/z = 474.96 (C₂₃H₁₅Br₂N = 477.20) |
| Sub 1-A-23 | m/z = 550.99 (C₃₀H₁₉Br₂N = 553.30) |
| Sub 1-A-24 | m/z = 580.94 (C₃₀H₁₇Br₂N = 580.34) |
| Sub 1-A-25 | m/z = 477.94 (C₂₁H₁₂Br₂N₄ = 480.16) |
| Sub 1-A-26 | m/z = 630.01 (C [symbol] H₂₀Br₂N₄ = 632.36) |
| Sub 1-A-27 | m/z = 574.99 (C₃₂H₁₉Br₂N = 577.32) |
| Sub 1-A-28 | m/z = 550.99 (C₃₀H₁₉Br₂N = 553.30) |
| Sub 1-A-29 | m/z = 524.97 (C₂₈H₁₇Br₂N = 527.26) |
| Sub 1-A-30 | m/z = 524.97 (C₂₈H₁₇Br₂N = 527.26) |
| Sub 1-A-31 | m/z = 574.99 (C₃₂H₁₉Br₂N = 577.32) |
| Sub 1-A-32 | m/z = 513.11 (C₃₃H₂₄BrN = 514.47) |
| Sub 1-B-1 | m/z = 321.02 (C₁₈H₁₂BrN = 322.20) |
| Sub 1-B-2 | m/z = 397.05 (C₂₄H₁₆BrN = 398.29) |
| Sub 1-B-3 | m/z = 549.11 (C₃₀H₂₄BrN = 550.49) |
| Sub 1-B-4 | m/z = 550.10 (C₃₅H₂₃BrN₂ = 551.47) |
| Sub 1-B-5 | m/z = 473.08 (C₃₀H₂₆BrN = 474.39) |
| Sub 1-B-6 | m/z = 474.07 (C₂₉H₁₉BrN₂ = 475.38) |
| Sub 1-B-7 | m/z = 447.06 (C₂₈H₁₈BrN = 448.35) |
| Sub 1-B-8 | m/z = 447.06 (C₂₈H₁₈BrN = 448.35) |
| Sub 1-B-9 | m/z = 447.06 (C₂₆H₁₉BrN = 448.35) |
| Sub 1-B-10 | m/z = 497.08 (C₃₂H₂₉BrN = 498.41) |
| Sub 1-B-11 | m/z = 497.08 (C₃₂H₂₀BrN = 498.41) |
| Sub 1-B-12 | m/z = 397.05 (C₂₄H₁₆BrN = 398.29) |
| Sub 1-B-13 | m/z = 638.14 (C₄₂H [symbol] BrN₂ = 639.58) |
| Sub 1-B-14 | m/z = 579.07 (C₃₆H₂₂BrNS = 580.54) |
| Sub 1-B-15 | m/z = 451.09 (C₃₀H₂₂BrN = 452.38) |
| Sub 1-B-16 | m/z = 564.12 (C₃₆H₂₅BrN₂ = 565.50) |
| Sub 1-B-17 | m/z = 447.06 (C₂₈H₁₈BrN = 448.35) |
| Sub 1-B-18 | m/z = 397.05 (C₂₄H₁₆BrN = 398.29) |
| Sub 1-B-19 | m/z = 397.05 (C₂₄H₁₆BrN = 398.29) |
| Sub 1-B-20 | m/z = 447.06 (C₂₅H₁₈BrN = 448.35) |
| Sub 1-B-21 | m/z = 497.08 (C₃₂H₂₈BrN = 498.41) |
| Sub 1-B-22 | m/z = 397.05 (C [symbol] H₁₆BrN = 398.29) |
| Sub 1-B-23 | m/z = 397.05 (C₂₆H₁₆BrN = 398.29) |
| Sub 1-B-24 | m/z = 421.05 (C₂₆H₁₆BrN = 422.32) |
| Sub 1-B-25 | m/z = 447.06 (C₂₈H₁₈BrN = 448.35) |
| Sub 1-B-26 | m/z = 473.08 (C₃₀H₂₀BrN = 474.39) |
| Sub 1-B-27 | m/z = 625.14 (C₄₂H₂₈BrN = 626.58) |
| Sub 1-B-28 | m/z = 548.09 (C₃₅H₂₇BrN₂ = 549.46) |
| Sub 1-B-29 | m/z = 427.00 (C₂₄H₁₄BrNS = 428.34) |
| Sub 1-B-30 | m/z = 527.03 (C₃₂H₁₈BrNS = 528.46) |
| Sub 1-B-31 | m/z = 427.00 (C₂₄H₁₄BrNS = 428.34) |
| Sub 1-B-32 | m/z = 427.00 (C₂₄H₁₄BrNS = 428.34) |
| Sub 1-B-33 | m/z = 411.03 (C₂₄H₁₄BrNO = 412.28) |
| Sub 1-B-34 | m/z = 411.03 (C₂₄H₁₄BrNO = 412.28) |
| Sub 1-B-35 | m/z = 437.08 (C₂₇H₂₀BrN = 438.36) |
| Sub 1-B-36 | m/z = 563.12 (C₃₇H₂₆BrN = 564.51) |
| Sub 1-B-37 | m/z = 590.14 (C₃₈H₂₇BrN₂ = 591.54) |
| Sub 1-B-38 | m/z = 467.09 (C₃₁H₂₂BrN = 488.42) |
| Sub 1-B-39 | m/z = 487.09 (C₃₁H₂₂BrN = 488.42) |
| Sub 1-B-40 | m/z = 487.09 (C₃₁H₂₂BrN = 488.42) |
| Sub 1-B-41 | m/z = 537.11 (C₃₅H₂₄BrN = 538.48) |
| Sub 1-B-42 | m/z = 537.11 (C₃₅H₂₄BrN = 538.48) |
| Sub 1-B-43 | m/z = 437.08 (C₂₇H₂₀BrN = 438.36) |
| Sub 1-B-44 | m/z = 561.11 (C₃₇H₂₄BrN = 562.50) |
| Sub 1-B-45 | m/z = 561.11 (C₃₇H₂₄BrN = 562.50) |
| Sub 1-B-46 | m/z = 559.09 (C₃₇H₂₂BrN = 560.48) |
| Sub 1-B-47 | m/z = 559.09 (C₃₇H₂₂BrN = 560.48) |
| Sub 1-B-48 | m/z = 762.17 (C₅₂H₃₁BrN₂ = 763.72) |
| Sub 1-A-33 | m/z = 261.95 (C₁₂H₇BrS = 263.15) |
| Sub 1-A-34 | m/z = 337.98 (C₁₈H₁₁BrS = 339.35) |
| Sub 1-A-35 | m/z = 414.01 (C₂₄H₁₅BrS = 415.35) |
| Sub 1-A-36 | m/z = 454.04 (C₂₇H₁₉BrS = 455.41) |
| Sub 1-A-37 | m/z = 337.98 (C₁₈H₁₁BrS = 339.25) |
| Sub 1-A-38 | m/z = 337.98 (C₁₈H₁₁BrS = 339.25) |
| Sub 1-A-39 | m/z = 387.99 (C₂₈H₁₃BrS = 389.31) |
| Sub 1-A-40 | m/z = 464.02 (C₂₈H₁₇BrS = 465.41) |
| Sub 1-A-41 | m/z = 438.10 (C₂₆H₁₅BrS = 439.37) |
| Sub 1-A-42 | m/z = 387.99 (C₂₂H₁₃BrS = 389.31) |
| Sub 1-A-43 | m/z = 311.96 (C₁₆H₉BrS = 313.21) |
| Sub 1-A-44 | m/z = 414.01 (C₂₄H₁₅BrS = 415.35) |
| Sub 1-A-45 | m/z = 569.06 (C₃₅H₂₀BrN₃S = 570.51) |
| Sub 1-A-46 | m/z = 570.07 (C₃₆H₂₂BrNS = 580.54) |
| Sub 1-A-47 | m/z = 387.99 (C₂₂H₁₃BrS = 389.31) |
| Sub 1-A-48 | m/z = 311.96 (C₁₆H₉BrS = 313.21) |
| Sub 1-A-49 | m/z = 378.01 (C₂₉H₁₅BrS = 379.32) |
| Sub 1-A-50 | m/z = 438.01 (C₂₉H₁₅BrS = 439.37) |
| Sub 1-A-51 | m/z = 398.03 (C₂₄H₁₅BrO = 399.29) |
| Sub 1-A-52 | m/z = 438.06 (C₂₇H₁₉BrO = 439.35) |
| Sub 1-A-53 | m/z = 245.97 (C₁₂H₇BrO = 247.09) |
| Sub 1-A-54 | m/z = 322 (C₁₈H₁₁BrO = 323.19) |
| Sub 1-A-55 | m/z = 372.01 (C₂₂H₁₃BrO = 373.25) |
| Sub 1-A-56 | m/z = 422.03 (C₂₆H₁₅BrO = 423.31) |
| Sub 1-A-57 | m/z = 295.39 (C₁₆H₉BrO = 297.15) |
| Sub 1-A-58 | m/z = 322 (C₁₈H₁₁BrO = 323.19) |
| Sub 1-A-59 | m/z = 322 (C₁₈H₁₁BrO = 323.19) |
| Sub 1-A-60 | m/z = 322 (C₁₈H₁₁BrO = 323.19) |
| Sub 1-A-61 | m/z = 398.03 (C₂₄H₁₅BrO = 399.29) |
| Sub 1-A-62 | m/z = 563.09 (C₃₆H₂₂BrNO = 564.48) |
| Sub 1-A-63 | m/z = 228.07 (C₁₅H₁₃Cl = 228.72) |
| Sub 1-A-64 | m/z = 228.07 (C₁₅H₁₃Cl = 228.72) |
| Sub 1-A-65 | m/z = 228.07 (C₁₅H₁₃Cl = 228.72) |
| Sub 1-A-66 | m/z = 380.13 (C₂₇H₂₁Cl = 380.92) |
| Sub 1-A-67 | m/z = 348.05 (C₂₁H₁₇Br = 349.27) |
| Sub 1-A-68 | m/z = 424.08 (C₂₇H₂₁Br = 425.37) |
| Sub 1-A-69 | m/z = 500.11 (C₃₃H₂₅Br = 501.47) |
| Sub 1-A-70 | m/z = 348.05 (C₂₁H₁₇Br = 349.27) |
| Sub 1-A-71 | m/z = 500.11 (C [symbol] H₂₅Br = 501.47) |
| Sub 1-A-72 | m/z = 464.11 (C₃₀H₂₅Br = 465.43) |
| Sub 1-A-73 | m/z = 448.08 (C₂₈H₂₁Br = 449.39) |
| Sub 1-A-74 | m/z = 352.10 (C₂₅H₁₇Cl = 352.86) |
| Sub 1-A-75 | m/z = 462.09 (C₃₁H₂₉Cl₂ = 463.40) |
| Sub 1-A-76 | m/z = 352.10 (C₂₅H₁₇Cl = 352.86) |
| Sub 1-A-77 | m/z = 352.10 (C₂₅H₁₇Cl = 352.86) |
| Sub 1-A-78 | m/z = 402.12 (C29H19Cl = 402.92) |
| Sub 1-A-79 | m/z = 352.10 (C₂₅H₁₇Cl = 352.66) |
| Sub 1-A-80 | m/z = 472.08 (C₃₁H₂₁Br = 473.41) |
| Sub 1-A-81 | m/z = 550.13 (C₃₇H₂₇Br = 551.53) |
| Sub 1-A-82 | m/z = 548.11 (C₃₇H₂₅Br = 549.51) |
| Sub 1-A-83 | m/z = 472.08 (C₃₁H₂₁Br = 473.41) |
| Sub 1-A-84 | m/z = 585.09 (C₃₆H₂₂BrF₂N = 586.48) |
| Sub 1-A-85 | m/z = 394.04 (C₂₅H₁₅Br = 395.30) |
| Sub 1-A-86 | m/z = 350.09 (C₂₅H₁₅Cl = 350.85) |
| Sub 1-A-87 | m/z = 350.09 (C₂₅H₁₅Cl = 350.85) |
| Sub 1-A-88 | m/z = 470.07 (C₃₁H₁₉Br = 471.40) |
| Sub 1-A-89 | m/z = 470.07 (C₃₃H₁₉Br = 471.40) |
| Sub 1-A-90 | m/z = 470.07 (C₃₁H₁₉Br = 471.40) |
| Sub 1-A-91 | m/z = 470.07 (C₃₃H₁₉Br = 471.40) |
| Sub 1-A-92 | m/z = 504.03 (C₃₁H₁₈BrCl = 505.84) |
| Sub 1-A-93 | m/z = 470.07 (C₃₁H₁₉Br = 471.40) |
| Sub 1-A-94 | m/z = 546.10 (C₃₇H₂₃Br = 547.50) |
| Sub 1-A-95 | m/z = 400.10 (C₂₉H₁₇Cl = 400.91) |
| Sub 1-A-96 | m/z = 476.13 (C₃₅H₂₁Cl = 477.00) |
| Sub 1-A-97 | m/z = 400.10 (C₂₉H₁₇Cl = 400.91) |
| Sub 1-A-98 | m/z = 400.10 (C₂₉H₁₇Cl = 400.91) |
| Sub 1-A-99 | m/z = 400.10 (C₂₉H₁₇Cl = 400.91) |
| Sub 1-A-100 | m/z = 400.10 (C₂₉H₁₇Cl = 400.91) |
| Sub 1-A-101 | m/z = 400.10 (C₂₉H₁₇Cl = 400.91) |
| Sub 1-A-102 | m/z = 400.10 (C₂₉H₁₇Cl = 400.91) |
| Sub 1-A-103 | m/z = 478.12 (C₃₃H₁₉ClN₂ = 478.98) |
| Sub 1-A-104 | m/z = 400.10 (C₂₉H₁₇Cl = 400.91) |
| Sub 1-A-105 | m/z = 400.10 (C₂₉H₁₇Cl = 400.91) |
| Sub 1-A-106 | m/z = 400.10 (C₂₉H₁₇Cl = 400.91) |
| Sub 1-A-107 | m/z = 400.10 (C₂₉H₁₇Cl = 400.91) |
| Sub 1-A-108 | m/z = 400.10 (C₂₉H₁₇Cl = 400.91) |
| Sub 1-A-109 | m/z = 526.15 (C₃₉H₂₃Cl = 527.06) |
| Sub 1-A-110 | m/z = 400.10 (C₂₉H₁₇Cl = 400.91) |
| Sub 1-A-111 | m/z = 400.10 (C₂₉H₁₇Cl = 400.91) |
| Sub 1-A-112 | m/z = 400.10 (C₂₉H₁₇Cl = 400.91) |
| Sub 1-A-113 | m/z = 566.14 (C₄₁H₂₃ClO = 567.08) |
| Sub 1-A-114 | m/z = 400.10 (C₂₉H₁₇Cl = 400.91) |
| Sub 1-A-115 | m/z = 400.10 (C₂₉H₁₇Cl = 400.91) |
| Sub 1-A-116 | m/z = 400.10 (C₂₉H₁₇Cl = 400.91) |
| Sub 1-A-117 | m/z = 400.10 (C₂₉H₁₇Cl = 400.91) |
| Sub 1-A-118 | m/z = 400.10 (C₂₉H₁₇Cl = 400.91) |
| Sub 1-A-119 | m/z = 400.10 (C₂₉H₁₇Cl = 400.91) |
| Sub 1-A-120 | m/z = 400.10 (C₂₉H₁₇Cl = 400.91) |
| Sub 1-A-121 | m/z = 400.10 (C₂₉H₁₇Cl = 400.91) |
| Sub 1-A-122 | m/z = 400.10 (C₂₉H₁₇Cl = 400.91) |
| Sub 1-A-123 | m/z = 425.10 (C₃₀H₁₆ClN = 425.92) |
| Sub 1-A-124 | m/z = 400.10 (C₂₉H₁₇Cl = 400.91) |
| Sub 1-A-125 | m/z = 400.10 (C₂₉H₁₇Cl = 400.91) |
| Sub 1-A-126 | m/z = 400.10 (C₂₉H₁₇Cl = 400.91) |
| Sub 1-A-127 | m/z = 400.10 (C₂₉H₁₇Cl = 400.91) |
| Sub 1-A-128 | m/z = 400.10 (C₂₉H₁₇Cl = 400.91) |
| Sub 1-A-129 | m/z = 400.10 (C₂₉H₁₇Cl = 400.91) |
| Sub 1-A-130 | m/z = 400.10 (C₂₉H₁₇Cl = 400.91) |
| Sub 1-A-131 | m/z = 400.10 (C₂₉H₁₇Cl = 400.91) |
| Sub 1-A-132 | m/z = 520.08 (C₃₅H₂₁Br = 521.46) |
| Sub 1-A-133 | m/z = 520.08 (C₃₅H₂₁Br = 521.46) |
| Sub 1-A-134 | m/z = 520.08 (C₃₅H₂₁Br = 521.46) |
| Sub 1-A-135 | m/z = 510.09 (C₃₅H₂₀Cl₂ = 511.45) |
| Sub 1-A-136 | m/z = 610.09 (C₄₁H₂₁BrO = 611.54) |
| Sub 1-A-137 | m/z = 626.07 (C₄₁H₂₃BrS = 627.60) |
| Sub 1-A-138 | m/z = 636.15 (C₄₄H₂₉Br = 637.62) |
| Sub 1-A-139 | m/z = 450.12 (C₃₃H₁₉Cl = 450.97) |
| Sub 1-A-140 | m/z = 450.12 (C₃₃H₁₉Cl = 450.97) |
| Sub 1-A-141 | m/z = 500.13 (C₃₇H₂₁Cl = 501.03) |
| Sub 1-A-142 | m/z = 450.12 (C₃₃H₁₉Cl = 450.97) |
| Sub 1-A-143 | m/z = 450.12 (C₃₃H₁₉Cl = 450.97) |
| Sub 1-A-144 | m/z = 670.13 (C₄₇H₂₇Br = 671.64) |
| Sub 1-A-145 | m/z = 696.15 (C₄₉H₂₉Br = 697.68) |
| Sub 1-A-146 | m/z = 772.15 (C₅₃H₂₉BrN₂ = 773.73) |
| Sub 1-A-148 | m/z = 348.05 (C₂₁H₁₇Br = 349.27) |
| Sub 1-A-149 | m/z = 424.08 (C₂₇H₂₁Br = 425.37) |
| Sub 1-A-152 | m/z = 396.05 (C₂₅H₁₇Br = 397.32) |
| Sub 1-A-154 | m/z = 322.04 (C₁₉H₁₅Br = 323.23) |
| Sub 1-A-155 | m/z = 231.99 (C12H9Br = 233.11) |
| Sub 1-A-156 | m/z = 282 (C16H11Br = 283.17) |
| Sub 1-A-157 | m/z = 332.02 (C20H13Br = 333.23) |
| Sub 1-A-161 | m/z = 384.05 (C24H17Br = 385.3) |

[symbol]

indicates data missing or illegible when filed

Illustration of Synthesis of Sub 2

Sub 2 of Reaction Formula 1 may be synthesized through, but is not limited to, a reaction path of the following Reaction Formula 8.

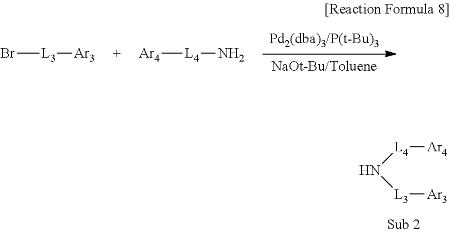

Synthesis of Sub 2-1

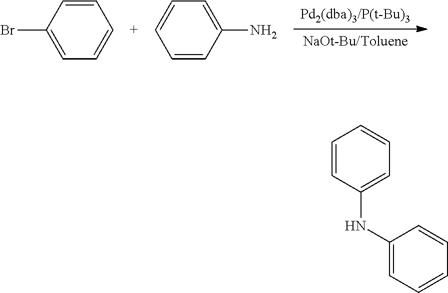

Bromobenzene (37.1 g, 236.2 mmol) was added to a round bottom flask, and then dissolved with toluene (2200 ml). Afterwards, aniline (20 g, 214.8 mmol), Pd₂(dba)₃ (9.83 g, 10.7 mmol), P(t-Bu)₃ (4.34 g, 21.5 mmol), and NaOt-Bu (62 g, 644.3 mmol) were sequentially added, followed by stirring at 100° C. When the reaction was completed, extraction was performed with ether and water, and then an organic layer was dried with MgSO₄ and concentrated. A resultant compound was subjected to silica gel column chromatography and recrystallization, thereby creating a product 28 g (yield: 77%).

Synthesis of Sub 2-80

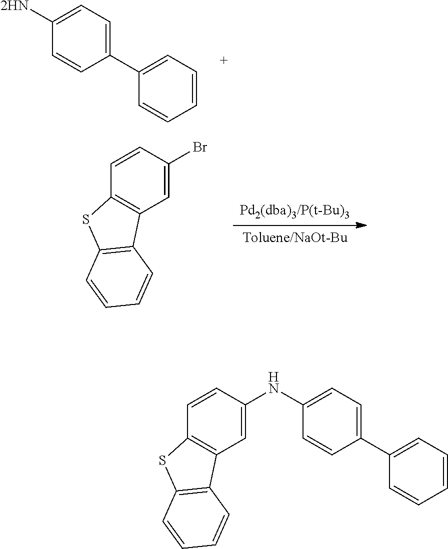

[1,1′-biphenyl]-4-amine (15 g, 88.64 mmol), 2-bromodibenzo[b,d]thiophene (23.32 g, 88.64 mmol), Pd₂(dba)₃ (2.43 g, 2.66 mmol), P(t-Bu)₃ (17.93 g, 88.64 mmol), NaOt-Bu (17.04 g, 177.27 mmol), and toluene (886 ml) were added to a round bottom flask, and then subjected to the same experimental method as in Sub 2-1, thereby creating a product 24.61 g (yield: 79%).

Synthesis of Sub 2-134

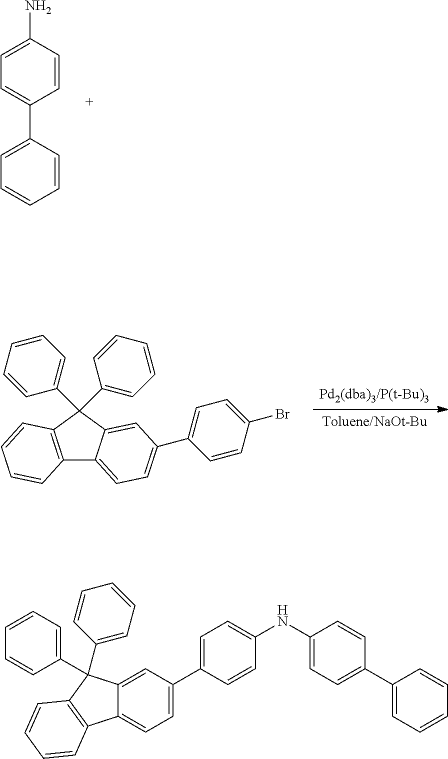 [SMILES: Nc1ccc(-c2ccccc2)cc1 ; Brc1ccc(-c2ccc3c(c2)C(c2ccccc2)(c2ccccc2)c2ccccc2-3)cc1 ; "Pd2(dba)3/P(t-Bu)3" ; "Toluene/NaOt-Bu" ; c1ccc(-c2ccc(Nc3ccc(-c4ccc5c(c4)C(c4ccccc4)(c4ccccc4)c4ccccc4-5)cc3)cc2)cc1]

[1,1′-biphenyl]-4-amine (15 g, 88.6 mmol), 2-(4-bromophenyl)-9,9-diphenyl-9H-fluorene (46.2 g, 97.5 mmol), Pd₂(dba)₃ (4.06 g, 4.43 mmol), P(t-Bu)₃ (1.8 g, 8.86 mmol), NaOt-Bu (28.1 g, 292.5 mmol), and toluene (931 ml) were added to a round bottom flask, and subjected to the same experimental method as in Sub 2-1, thereby creating a product 34.9 g (yield: 70%).

### Example of Synthesis of Sub 2-222

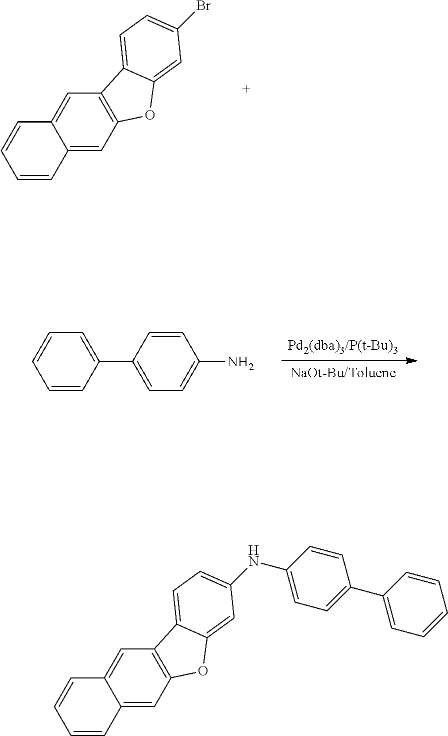 [SMILES: Brc1ccc2c(c1)oc1cc3ccccc3cc12 ; Nc1ccc(-c2ccccc2)cc1 ; "NaOt-Bu/Toluene" ; "Pd2(dba)3/P(t-Bu)3" ; c1ccc(-c2ccc(Nc3ccc4c(c3)oc3cc5ccccc5cc34)cc2)cc1]

3-bromonaphtho[2,3-b]benzofuran (15 g, 50.48 mmol), [1,1′-biphenyl]-4-amine (8.54 g, 50.48 mmol), Pd₂(dba)₃ (1.39 g, 1.51 mmol), P(t-Bu)₃ (10.21 g, 50.48 mmol), NaOt-Bu (9.70 g, 100.96 mmol), and toluene (505 ml) were added to a round bottom flask, and subjected to the same experimental method as in Sub 2-1, thereby creating a product 13.82 g (yield: 71%).

Sub 2 may have, but is not limited to, the following examples.

TABLE 2

| | |
|---|---|
| 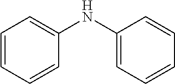 [SMILES: c1ccc(Nc2ccccc2)cc1] | Sub 2-1 |
| 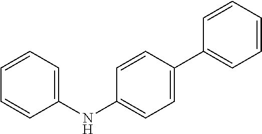 [SMILES: c1ccc(Nc2ccc(-c3ccccc3)cc2)cc1] | Sub 2-2 |
| 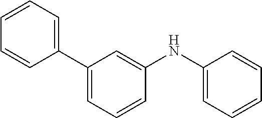 [SMILES: c1ccc(Nc2cccc(-c3ccccc3)c2)cc1] | Sub 2-3 |
| 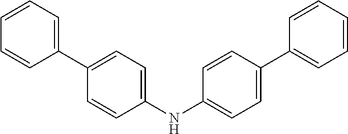 [SMILES: c1ccc(-c2ccc(Nc3ccc(-c4ccccc4)cc3)cc2)cc1] | Sub 2-4 |
| 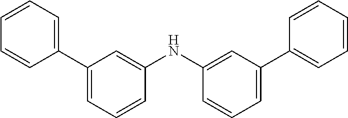 [SMILES: c1ccc(-c2cccc(Nc3cccc(-c4ccccc4)c3)c2)cc1] | Sub 2-5 |
| 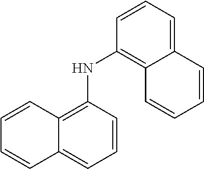 [SMILES: c1ccc2c(Nc3cccc4ccccc34)cccc2c1] | Sub 2-6 |
| 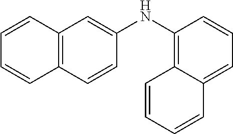 [SMILES: c1ccc2cc(Nc3cccc4ccccc34)ccc2c1] | Sub 2-7 |
| 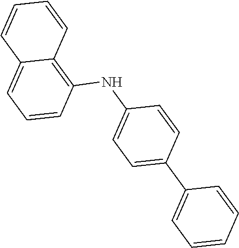 [SMILES: c1ccc(-c2ccc(Nc3cccc4ccccc34)cc2)cc1] | Sub 2-8 |
| 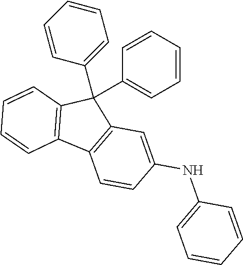 [SMILES: c1ccc(Nc2ccc3c(c2)C(c2ccccc2)(c2ccccc2)c2ccccc2-3)cc1] | Sub 2-9 |
| 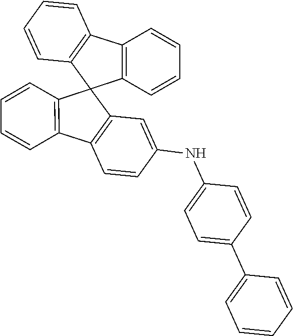 [SMILES: c1ccc(-c2ccc(Nc3ccc4c(c3)C3(c5ccccc5-c5ccccc53)c3ccccc3-4)cc2)cc1] | Sub 2-10 |
| 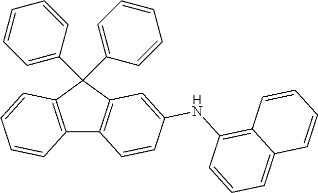 [SMILES: c1ccc(C2(c3ccccc3)c3ccccc3-c3ccc(Nc4cccc5ccccc45)cc32)cc1] | Sub 2-11 |
| 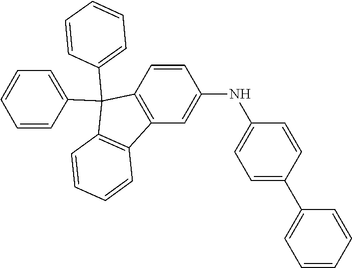 [SMILES: c1ccc(-c2ccc(Nc3ccc4c(c3)-c3ccccc3C4(c3ccccc3)c3ccccc3)cc2)cc1] | Sub 2-12 |
| 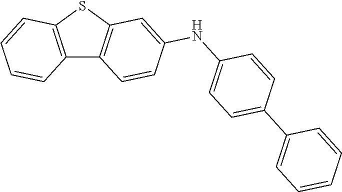 [SMILES: c1ccc(-c2ccc(Nc3ccc4c(c3)sc3ccccc34)cc2)cc1] | Sub 2-13 |
| 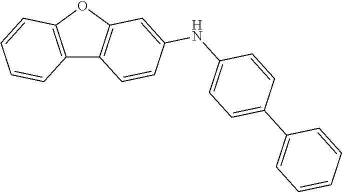 [SMILES: c1ccc(-c2ccc(Nc3ccc4c(c3)oc3ccccc34)cc2)cc1] | Sub 2-14 |
| 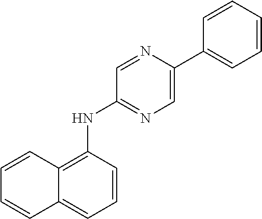 [SMILES: c1ccc(-c2cnc(Nc3cccc4ccccc34)cn2)cc1] | Sub 2-15 |
| 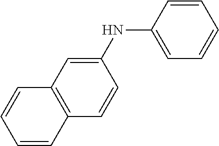 [SMILES: c1ccc(Nc2ccc3ccccc3c2)cc1] | Sub 2-16 |
| 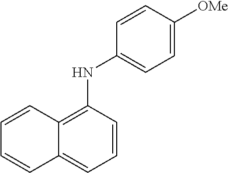 [SMILES: COc1ccc(Nc2cccc3ccccc23)cc1] | Sub 2-17 |
| 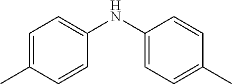 | Sub 2-18 |
| 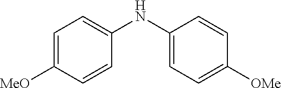 [SMILES: COc1ccc(Nc2ccc(OC)cc2)cc1] | Sub 2-19 |
| 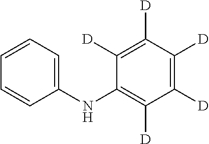 [SMILES: [2H]c1c([2H])c([2H])c(Nc2ccccc2)c([2H])c1[2H]] | Sub 2-20 |
| 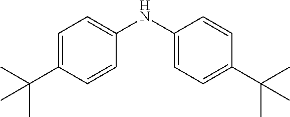 [SMILES: CC(C)(C)c1ccc(Nc2ccc(C(C)(C)C)cc2)cc1] | Sub 2-21 |
| 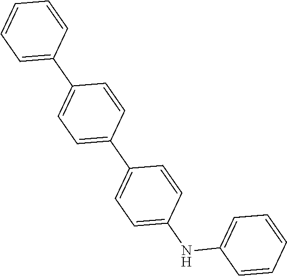 [SMILES: c1ccc(Nc2ccc(-c3ccc(-c4ccccc4)cc3)cc2)cc1] | Sub 2-22 |
| 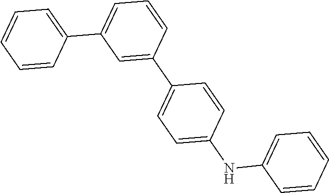 [SMILES: c1ccc(Nc2ccc(-c3cccc(-c4ccccc4)c3)cc2)cc1] | Sub 2-23 |
| 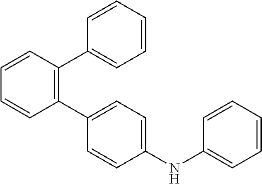 [SMILES: c1ccc(Nc2ccc(-c3ccccc3-c3ccccc3)cc2)cc1] | Sub 2-24 |
| 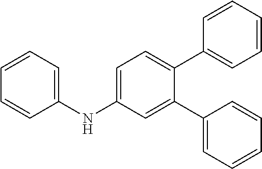 [SMILES: c1ccc(Nc2ccc(-c3ccccc3)c(-c3ccccc3)c2)cc1] | Sub 2-25 |
| 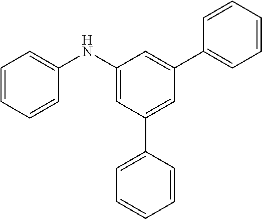 [SMILES: c1ccc(Nc2cc(-c3ccccc3)cc(-c3ccccc3)c2)cc1] | Sub 2-26 |
| 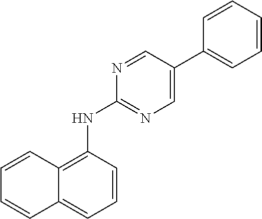 [SMILES: c1ccc(-c2cnc(Nc3cccc4ccccc34)nc2)cc1] | Sub 2-27 |
| 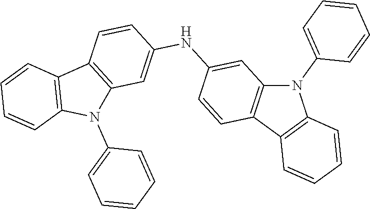 [SMILES: c1ccc(-n2c3ccccc3c3ccc(Nc4ccc5c6ccccc6n(-c6ccccc6)c5c4)cc32)cc1] | Sub 2-28 |
| 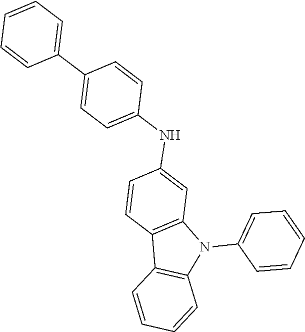 [SMILES: c1ccc(-c2ccc(Nc3ccc4c5ccccc5n(-c5ccccc5)c4c3)cc2)cc1] | Sub 2-29 |
| 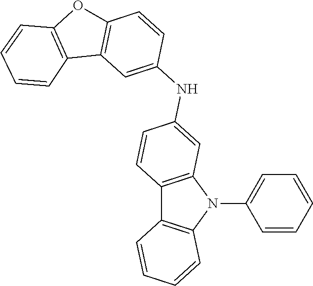 [SMILES: c1ccc(-n2c3ccccc3c3ccc(Nc4ccc5oc6ccccc6c5c4)cc32)cc1] | Sub 2-30 |
| 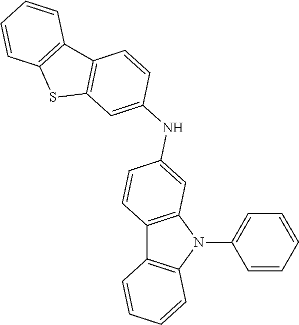 [SMILES: c1ccc(-n2c3ccccc3c3ccc(Nc4ccc5c(c4)sc4ccccc45)cc32)cc1] | Sub 2-31 |
| 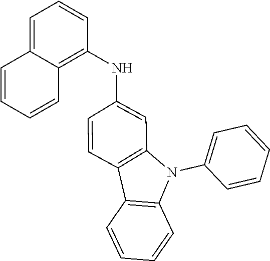 [SMILES: c1ccc(-n2c3ccccc3c3ccc(Nc4cccc5ccccc45)cc32)cc1] | Sub 2-32 |
| 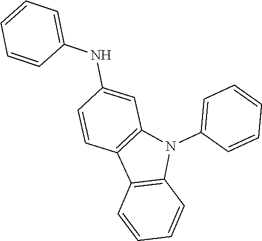 [SMILES: c1ccc(Nc2ccc3c4ccccc4n(-c4ccccc4)c3c2)cc1] | Sub 2-33 |
| 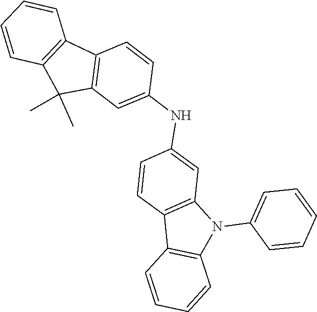 [SMILES: CC1(C)c2ccccc2-c2ccc(Nc3ccc4c5ccccc5n(-c5ccccc5)c4c3)cc21] | Sub 2-34 |
| 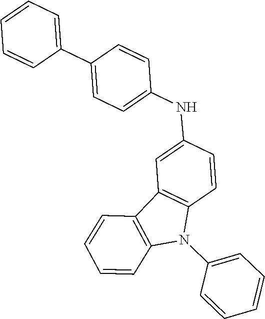 [SMILES: c1ccc(-c2ccc(Nc3ccc4c(c3)c3ccccc3n4-c3ccccc3)cc2)cc1] | Sub 2-35 |
| 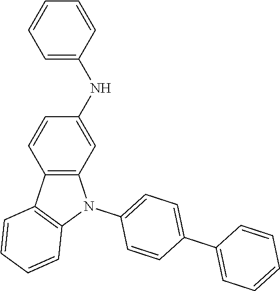 [SMILES: c1ccc(Nc2ccc3c4ccccc4n(-c4ccc(-c5ccccc5)cc4)c3c2)cc1] | Sub 2-36 |
| 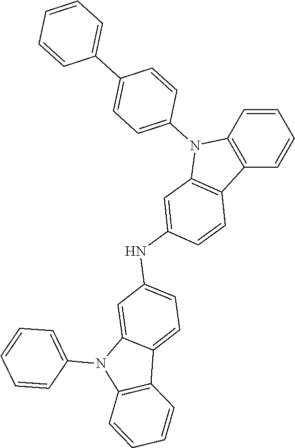 [SMILES: c1ccc(-c2ccc(-n3c4ccccc4c4ccc(Nc5ccc6c7ccccc7n(-c7ccccc7)c6c5)cc43)cc2)cc1] | Sub 2-37 |
| 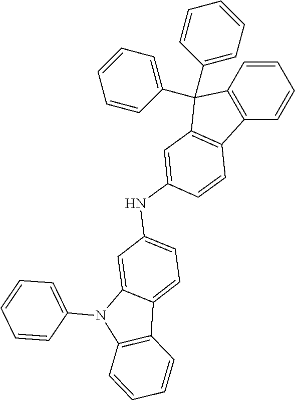 [SMILES: c1ccc(-n2c3ccccc3c3ccc(Nc4ccc5c(c4)C(c4ccccc4)(c4ccccc4)c4ccccc4-5)cc32)cc1] | Sub 2-38 |
| 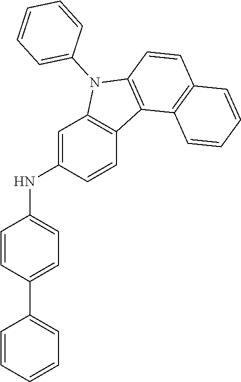 [SMILES: c1ccc(-c2ccc(Nc3ccc4c5c6ccccc6ccc5n(-c5ccccc5)c4c3)cc2)cc1] | Sub 2-39 |
| 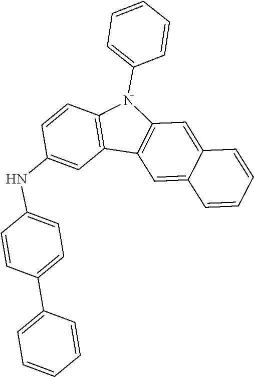 [SMILES: c1ccc(-c2ccc(Nc3ccc4c(c3)c3cc5ccccc5cc3n4-c3ccccc3)cc2)cc1] | Sub 2-40 |
| 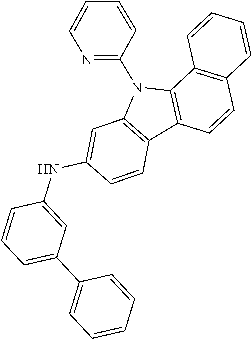 [SMILES: c1ccc(-c2cccc(Nc3ccc4c5ccc6ccccc6c5n(-c5ccccn5)c4c3)c2)cc1] | Sub 2-41 |
| 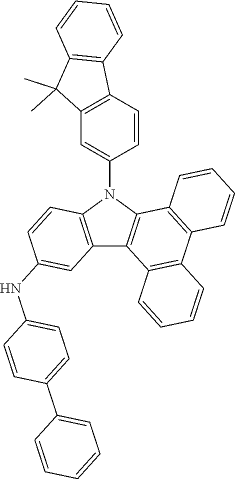 [SMILES: CC1(C)c2ccccc2-c2ccc(-n3c4ccc(Nc5ccc(-c6ccccc6)cc5)cc4c4c5ccccc5c5ccccc5c43)cc21] | Sub 2-42 |
| 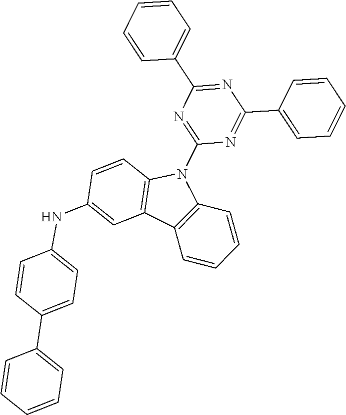 [SMILES: c1ccc(-c2ccc(Nc3ccc4c(c3)c3ccccc3n4-c3nc(-c4ccccc4)nc(-c4ccccc4)n3)cc2)cc1] | Sub 2-43 |
| 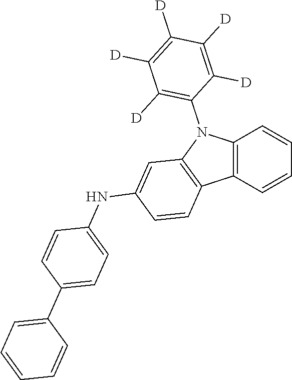 [SMILES: [2H]c1c([2H])c([2H])c(-n2c3ccccc3c3ccc(Nc4ccc(-c5ccccc5)cc4)cc32)c([2H])c1[2H]] | Sub 2-44 |
| 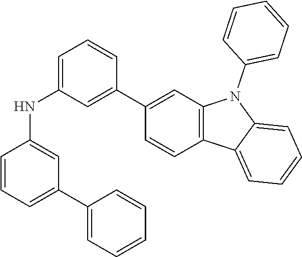 [SMILES: c1ccc(-c2cccc(Nc3cccc(-c4ccc5c6ccccc6n(-c6ccccc6)c5c4)c3)c2)cc1] | Sub 2-45 |
| 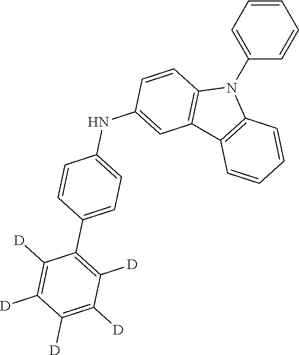 [SMILES: [2H]c1c([2H])c([2H])c(-c2ccc(Nc3ccc4c(c3)c3ccccc3n4-c3ccccc3)cc2)c([2H])c1[2H]] | Sub 2-46 |
| 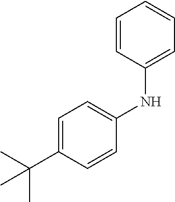 [SMILES: CC(C)(C)c1ccc(Nc2ccccc2)cc1] | Sub 2-47 |
| 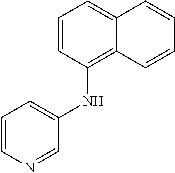 [SMILES: c1cncc(Nc2cccc3ccccc23)c1] | Sub 2-48 |
| 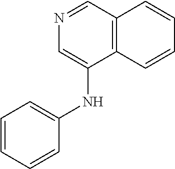 [SMILES: c1ccc(Nc2cncc3ccccc23)cc1] | Sub 2-49 |
| 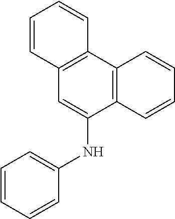 [SMILES: c1ccc(Nc2cc3ccccc3c3ccccc23)cc1] | Sub 2-50 |
| 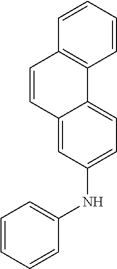 [SMILES: c1ccc(Nc2ccc3c(ccc4ccccc43)c2)cc1] | Sub 2-51 |
| 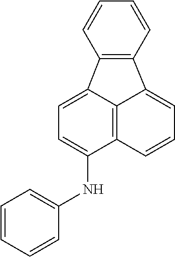 [SMILES: c1ccc(Nc2ccc3c4c(cccc24)-c2ccccc2-3)cc1] | Sub 2-52 |
| 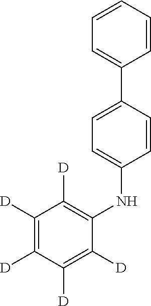 [SMILES: [2H]c1c([2H])c([2H])c(Nc2ccc(-c3ccccc3)cc2)c([2H])c1[2H]] | Sub 2-53 |
| 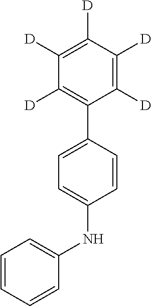 [SMILES: [2H]c1c([2H])c([2H])c(-c2ccc(Nc3ccccc3)cc2)c([2H])c1[2H]] | Sub 2-54 |
| 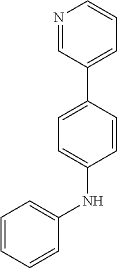 [SMILES: c1ccc(Nc2ccc(-c3cccnc3)cc2)cc1] | Sub 2-55 |
| 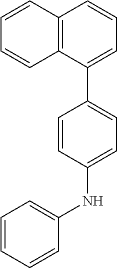 [SMILES: c1ccc(Nc2ccc(-c3cccc4ccccc34)cc2)cc1] | Sub 2-56 |
| 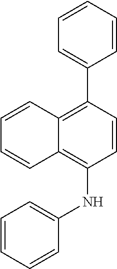 [SMILES: c1ccc(Nc2ccc(-c3ccccc3)c3ccccc23)cc1] | Sub 2-57 |
| 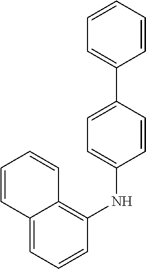 [SMILES: c1ccc(-c2ccc(Nc3cccc4ccccc34)cc2)cc1] | Sub 2-58 |
| 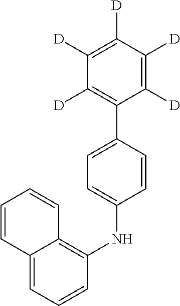 [SMILES: [2H]c1c([2H])c([2H])c(-c2ccc(Nc3cccc4ccccc34)cc2)c([2H])c1[2H]] | Sub 2-59 |
| 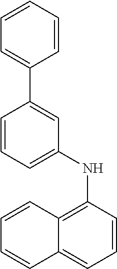 [SMILES: c1ccc(-c2cccc(Nc3cccc4ccccc34)c2)cc1] | Sub 2-60 |
| 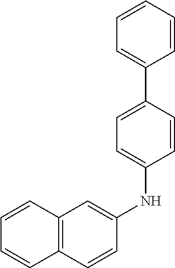 [SMILES: c1ccc(-c2ccc(Nc3ccc4ccccc4c3)cc2)cc1] | Sub 2-61 |
| 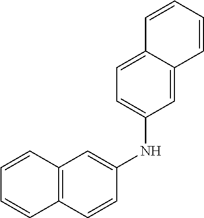 [SMILES: c1ccc2cc(Nc3ccc4ccccc4c3)ccc2c1] | Sub 2-62 |
| 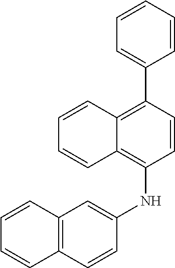 [SMILES: c1ccc(-c2ccc(Nc3ccc4ccccc4c3)c3ccccc23)cc1] | Sub 2-63 |
| 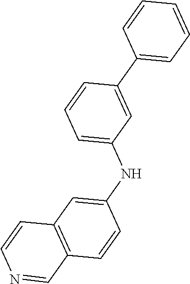 [SMILES: c1ccc(-c2cccc(Nc3ccc4cnccc4c3)c2)cc1] | Sub 2-64 |
| 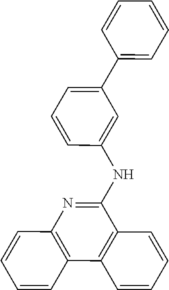 [SMILES: c1ccc(-c2cccc(Nc3nc4ccccc4c4ccccc34)c2)cc1] | Sub 2-65 |
| 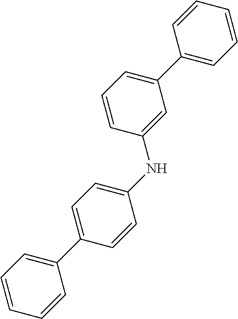 [SMILES: c1ccc(-c2ccc(Nc3cccc(-c4ccccc4)c3)cc2)cc1] | Sub 2-66 |
| 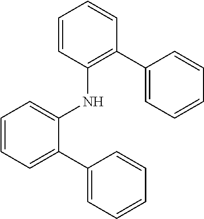 [SMILES: c1ccc(-c2ccccc2Nc2ccccc2-c2ccccc2)cc1] | Sub 2-67 |
| 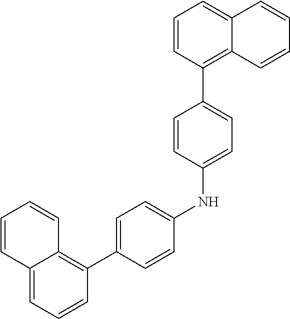 [SMILES: c1ccc2c(-c3ccc(Nc4ccc(-c5cccc6ccccc56)cc4)cc3)cccc2c1] | Sub 2-68 |
| 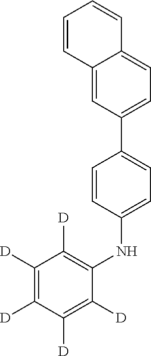 [SMILES: [2H]c1c([2H])c([2H])c(Nc2ccc(-c3ccc4ccccc4c3)cc2)c([2H])c1[2H]] | Sub 2-69 |
| 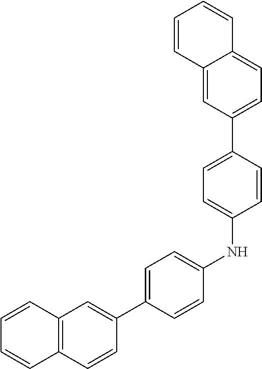 [SMILES: c1ccc2cc(-c3ccc(Nc4ccc(-c5ccc6ccccc6c5)cc4)cc3)ccc2c1] | Sub 2-70 |
| 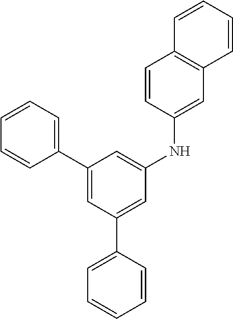 [SMILES: c1ccc(-c2cc(Nc3ccc4ccccc4c3)cc(-c3ccccc3)c2)cc1] | Sub 2-71 |
| 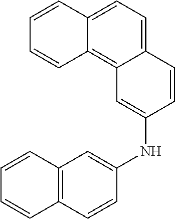 [SMILES: c1ccc2cc(Nc3ccc4ccc5ccccc5c4c3)ccc2c1] | Sub 2-72 |
| 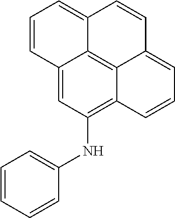 [SMILES: c1ccc(Nc2cc3cccc4ccc5cccc2c5c43)cc1] | Sub 2-73 |
| 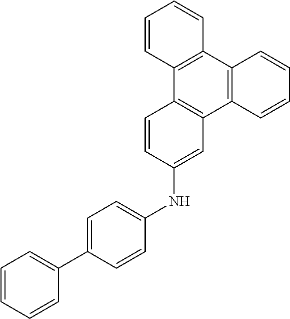 [SMILES: c1ccc(-c2ccc(Nc3ccc4c5ccccc5c5ccccc5c4c3)cc2)cc1] | Sub 2-74 |
| 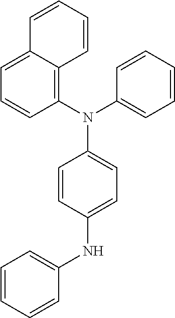 [SMILES: c1ccc(Nc2ccc(N(c3ccccc3)c3cccc4ccccc34)cc2)cc1] | Sub 2-75 |
| 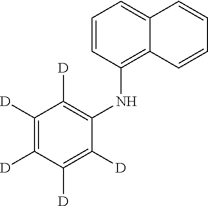 [SMILES: [2H]c1c([2H])c([2H])c(Nc2cccc3ccccc23)c([2H])c1[2H]] | Sub 2-76 |
| 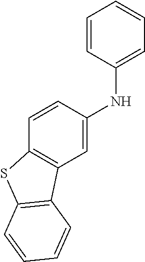 [SMILES: c1ccc(Nc2ccc3sc4ccccc4c3c2)cc1] | Sub 2-77 |
| 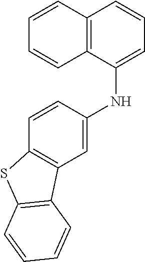 [SMILES: c1ccc2c(Nc3ccc4sc5ccccc5c4c3)cccc2c1] | Sub 2-78 |
| 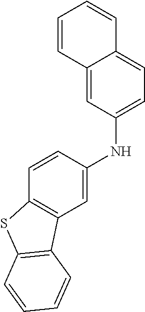 [SMILES: c1ccc2cc(Nc3ccc4sc5ccccc5c4c3)ccc2c1] | Sub 2-79 |
| 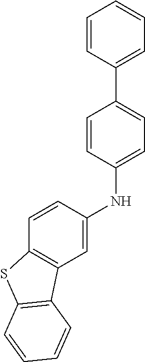 [SMILES: c1ccc(-c2ccc(Nc3ccc4sc5ccccc5c4c3)cc2)cc1] | Sub 2-80 |
| 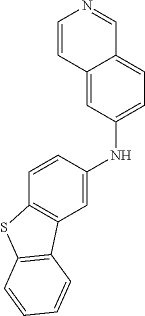 [SMILES: c1ccc2c(c1)sc1ccc(Nc3ccc4cnccc4c3)cc12] | Sub 2-81 |
| 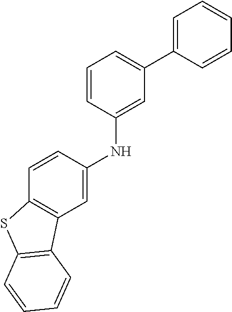 [SMILES: c1ccc(-c2cccc(Nc3ccc4sc5ccccc5c4c3)c2)cc1] | Sub 2-82 |
| 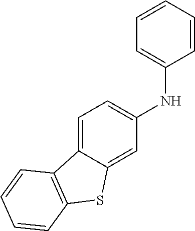 [SMILES: c1ccc(Nc2ccc3c(c2)sc2ccccc23)cc1] | Sub 2-83 |
| 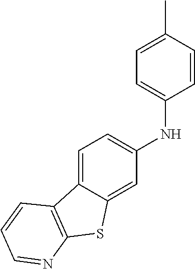 [SMILES: Cc1ccc(Nc2ccc3c(c2)sc2ncccc23)cc1] | Sub 2-84 |
| 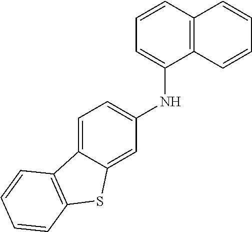 [SMILES: c1ccc2c(Nc3ccc4c(c3)sc3ccccc34)cccc2c1] | Sub 2-85 |
| 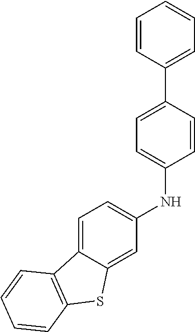 [SMILES: c1ccc(-c2ccc(Nc3ccc4c(c3)sc3ccccc34)cc2)cc1] | Sub 2-86 |
| 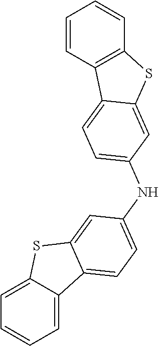 [SMILES: c1ccc2c(c1)sc1cc(Nc3ccc4c(c3)sc3ccccc34)ccc12] | Sub 2-87 |
| 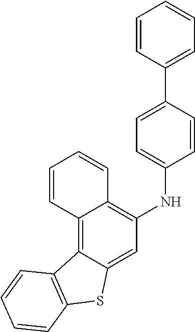 [SMILES: c1ccc(-c2ccc(Nc3cc4sc5ccccc5c4c4ccccc34)cc2)cc1] | Sub 2-88 |
| 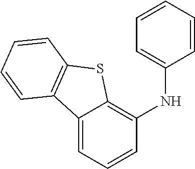 [SMILES: c1ccc(Nc2cccc3c2sc2ccccc23)cc1] | Sub 2-89 |
| 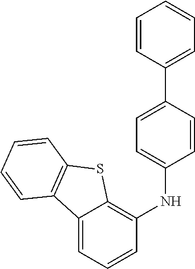 [SMILES: c1ccc(-c2ccc(Nc3cccc4c3sc3ccccc34)cc2)cc1] | Sub 2-90 |
| 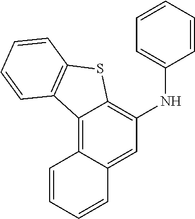 [SMILES: c1ccc(Nc2cc3ccccc3c3c2sc2ccccc23)cc1] | Sub 2-91 |
| 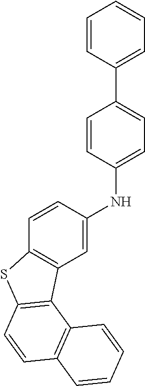 [SMILES: c1ccc(-c2ccc(Nc3ccc4sc5ccc6ccccc6c5c4c3)cc2)cc1] | Sub 2-92 |
| 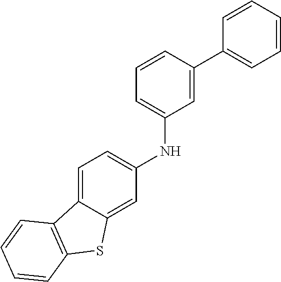 [SMILES: c1ccc(-c2cccc(Nc3ccc4c(c3)sc3ccccc34)c2)cc1] | Sub 2-93 |
| 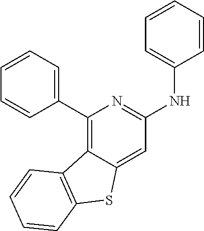 [SMILES: c1ccc(Nc2cc3sc4ccccc4c3c(-c3ccccc3)n2)cc1] | Sub 2-94 |
| 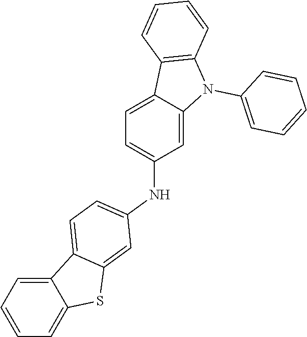 [SMILES: c1ccc(-n2c3ccccc3c3ccc(Nc4ccc5c(c4)sc4ccccc45)cc32)cc1] | Sub 2-95 |
| 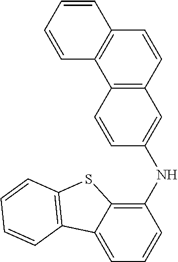 [SMILES: c1ccc2c(c1)ccc1cc(Nc3cccc4c3sc3ccccc34)ccc12] | Sub 2-96 |
| 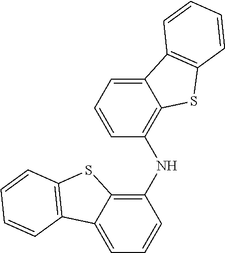 [SMILES: c1ccc2c(c1)sc1c(Nc3cccc4c3sc3ccccc34)cccc12] | Sub 2-97 |
| 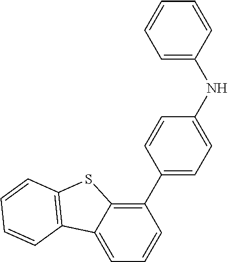 [SMILES: c1ccc(Nc2ccc(-c3cccc4c3sc3ccccc34)cc2)cc1] | Sub 2-98 |
| 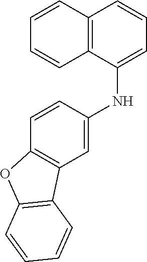 [SMILES: c1ccc2c(Nc3ccc4oc5ccccc5c4c3)cccc2c1] | Sub 2-99 |
| 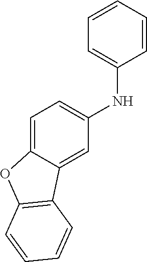 [SMILES: c1ccc(Nc2ccc3oc4ccccc4c3c2)cc1] | Sub 2-100 |
| 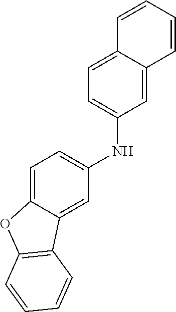 [SMILES: c1ccc2cc(Nc3ccc4oc5ccccc5c4c3)ccc2c1] | Sub 2-101 |
| 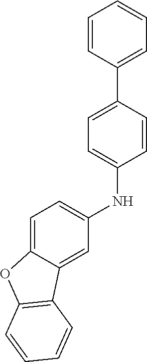 [SMILES: c1ccc(-c2ccc(Nc3ccc4oc5ccccc5c4c3)cc2)cc1] | Sub 2-102 |
| 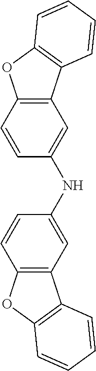 [SMILES: c1ccc2c(c1)oc1ccc(Nc3ccc4oc5ccccc5c4c3)cc12] | Sub 2-103 |
| 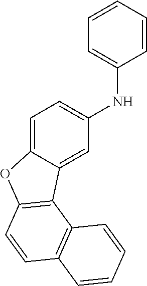 [SMILES: c1ccc(Nc2ccc3oc4ccc5ccccc5c4c3c2)cc1] | Sub 2-104 |
| 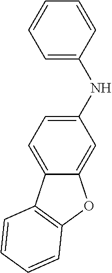 [SMILES: c1ccc(Nc2ccc3c(c2)oc2ccccc23)cc1] | Sub 2-105 |
| 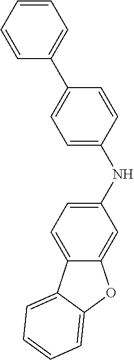 [SMILES: c1ccc(-c2ccc(Nc3ccc4c(c3)oc3ccccc34)cc2)cc1] | Sub 2-106 |
| 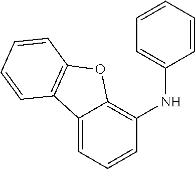 [SMILES: c1ccc(Nc2cccc3c2oc2ccccc23)cc1] | Sub 2-107 |
| 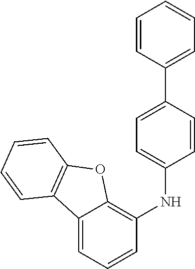 [SMILES: c1ccc(-c2ccc(Nc3cccc4c3oc3ccccc34)cc2)cc1] | Sub 2-108 |
| 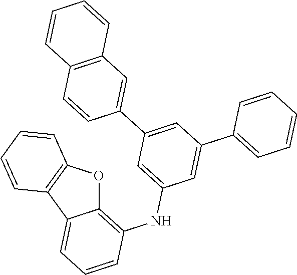 [SMILES: c1ccc(-c2cc(Nc3cccc4c3oc3ccccc34)cc(-c3ccc4ccccc4c3)c2)cc1] | Sub 2-109 |
| 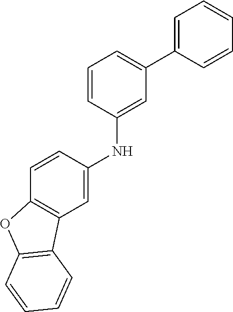 [SMILES: c1ccc(-c2cccc(Nc3ccc4oc5ccccc5c4c3)c2)cc1] | Sub 2-110 |
| 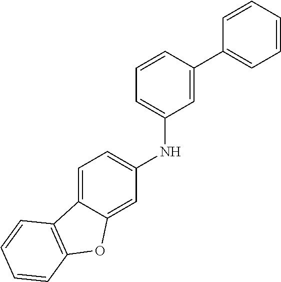 [SMILES: c1ccc(-c2cccc(Nc3ccc4c(c3)oc3ccccc34)c2)cc1] | Sub 2-111 |
| 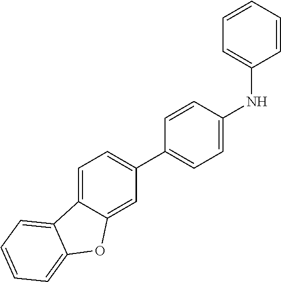 [SMILES: c1ccc(Nc2ccc(-c3ccc4c(c3)oc3ccccc34)cc2)cc1] | Sub 2-112 |
| 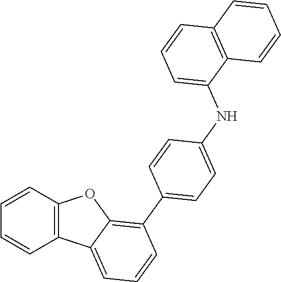 [SMILES: c1ccc2c(Nc3ccc(-c4cccc5c4oc4ccccc45)cc3)cccc2c1] | Sub 2-113 |
| 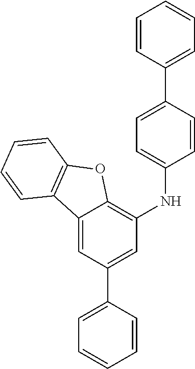 [SMILES: c1ccc(-c2ccc(Nc3cc(-c4ccccc4)cc4c3oc3ccccc34)cc2)cc1] | Sub 2-114 |
| 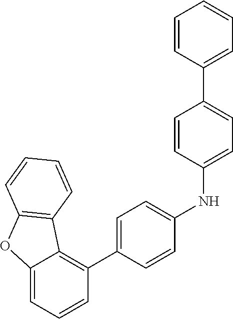 [SMILES: c1ccc(-c2ccc(Nc3ccc(-c4cccc5oc6ccccc6c45)cc3)cc2)cc1] | Sub 2-115 |
| 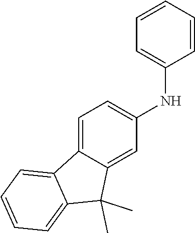 [SMILES: CC1(C)c2ccccc2-c2ccc(Nc3ccccc3)cc21] | Sub 2-116 |
| 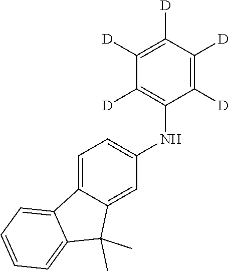 [SMILES: [2H]c1c([2H])c([2H])c(Nc2ccc3c(c2)C(C)(C)c2ccccc2-3)c([2H])c1[2H]] | Sub 2-117 |
| 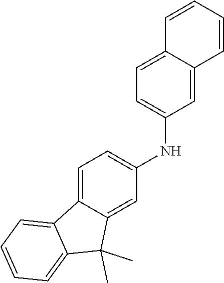 [SMILES: CC1(C)c2ccccc2-c2ccc(Nc3ccc4ccccc4c3)cc21] | Sub 2-118 |
| 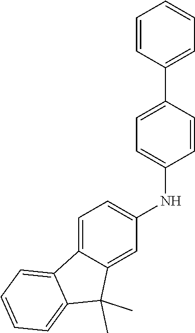 [SMILES: CC1(C)c2ccccc2-c2ccc(Nc3ccc(-c4ccccc4)cc3)cc21] | Sub 2-119 |
| 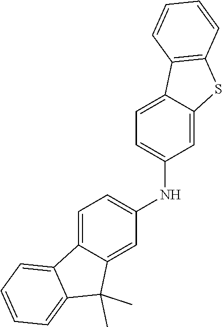 [SMILES: CC1(C)c2ccccc2-c2ccc(Nc3ccc4c(c3)sc3ccccc34)cc21] | Sub 2-120 |
| 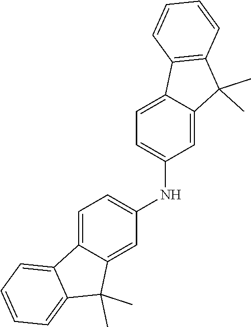 [SMILES: CC1(C)c2ccccc2-c2ccc(Nc3ccc4c(c3)C(C)(C)c3ccccc3-4)cc21] | Sub 2-121 |
| 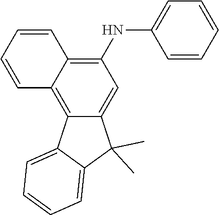 [SMILES: CC1(C)c2ccccc2-c2c1cc(Nc1ccccc1)c1ccccc21] | Sub 2-122 |
| 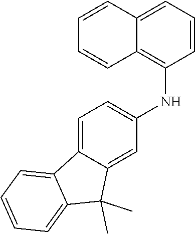 [SMILES: CC1(C)c2ccccc2-c2ccc(Nc3cccc4ccccc34)cc21] | Sub 2-123 |
| 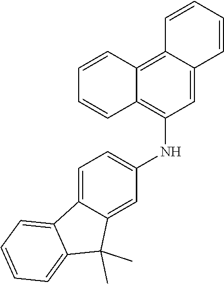 [SMILES: CC1(C)c2ccccc2-c2ccc(Nc3cc4ccccc4c4ccccc34)cc21] | Sub 2-124 |
| 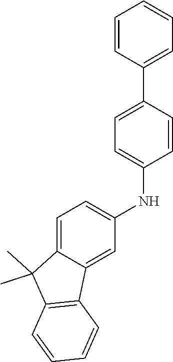 [SMILES: CC1(C)c2ccccc2-c2cc(Nc3ccc(-c4ccccc4)cc3)ccc21] | Sub 2-125 |
| 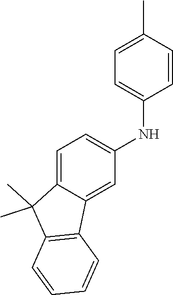 [SMILES: Cc1ccc(Nc2ccc3c(c2)-c2ccccc2C3(C)C)cc1] | Sub 2-126 |
| 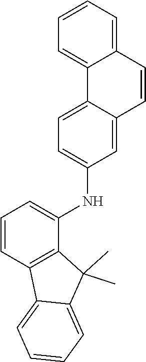 [SMILES: CC1(C)c2ccccc2-c2cccc(Nc3ccc4c(ccc5ccccc54)c3)c21] | Sub 2-127 |
| 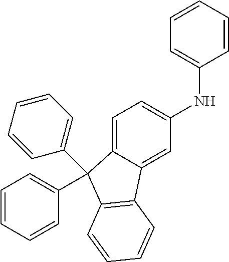 [SMILES: c1ccc(Nc2ccc3c(c2)-c2ccccc2C3(c2ccccc2)c2ccccc2)cc1] | Sub 2-128 |
| 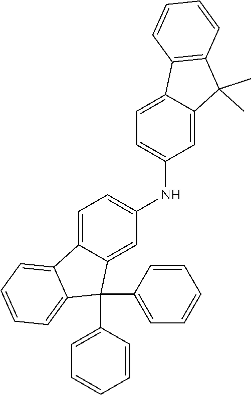 [SMILES: CC1(C)c2ccccc2-c2ccc(Nc3ccc4c(c3)C(c3ccccc3)(c3ccccc3)c3ccccc3-4)cc21] | Sub 2-129 |
| 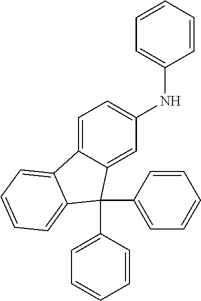 [SMILES: c1ccc(Nc2ccc3c(c2)C(c2ccccc2)(c2ccccc2)c2ccccc2-3)cc1] | Sub 2-130 |
| 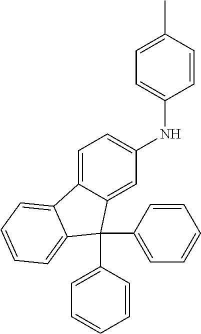 [SMILES: Cc1ccc(Nc2ccc3c(c2)C(c2ccccc2)(c2ccccc2)c2ccccc2-3)cc1] | Sub 2-131 |
| 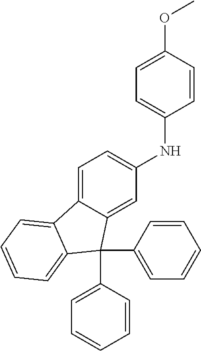 [SMILES: COc1ccc(Nc2ccc3c(c2)C(c2ccccc2)(c2ccccc2)c2ccccc2-3)cc1] | Sub 2-132 |
| 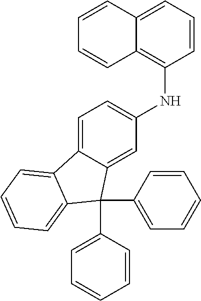 [SMILES: c1ccc(C2(c3ccccc3)c3ccccc3-c3ccc(Nc4cccc5ccccc45)cc32)cc1] | Sub 2-133 |
| 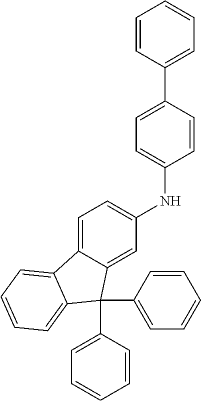 [SMILES: c1ccc(-c2ccc(Nc3ccc4c(c3)C(c3ccccc3)(c3ccccc3)c3ccccc3-4)cc2)cc1] | Sub 2-134 |
| 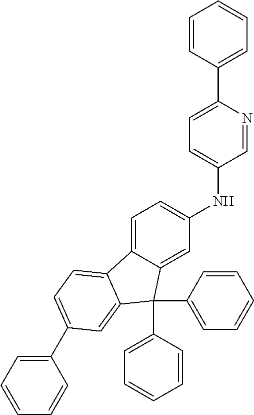 [SMILES: c1ccc(-c2ccc3c(c2)C(c2ccccc2)(c2ccccc2)c2cc(Nc4ccc(-c5ccccc5)nc4)ccc2-3)cc1] | Sub 2-135 |
| 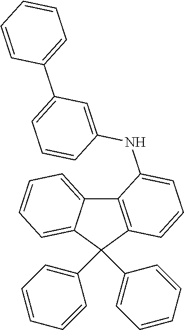 [SMILES: c1ccc(-c2cccc(Nc3cccc4c3-c3ccccc3C4(c3ccccc3)c3ccccc3)c2)cc1] | Sub 2-136 |
| 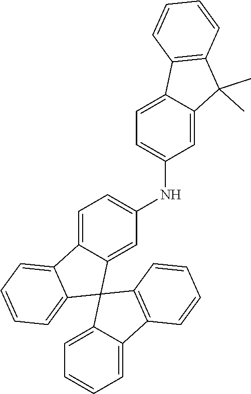 [SMILES: CC1(C)c2ccccc2-c2ccc(Nc3ccc4c(c3)C3(c5ccccc5-c5ccccc53)c3ccccc3-4)cc21] | Sub 2-137 |
| 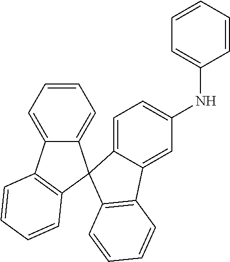 [SMILES: c1ccc(Nc2ccc3c(c2)-c2ccccc2C32c3ccccc3-c3ccccc32)cc1] | Sub 2-138 |
| 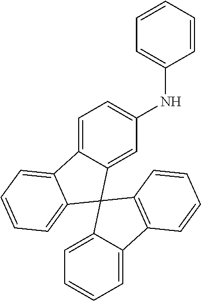 [SMILES: c1ccc(Nc2ccc3c(c2)C2(c4ccccc4-c4ccccc42)c2ccccc2-3)cc1] | Sub 2-139 |
| 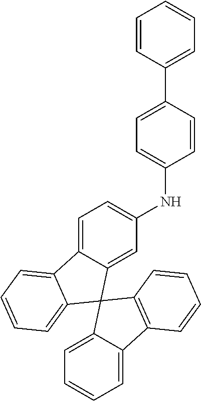 [SMILES: c1ccc(-c2ccc(Nc3ccc4c(c3)C3(c5ccccc5-c5ccccc53)c3ccccc3-4)cc2)cc1] | Sub 2-140 |
| 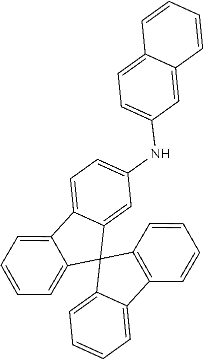 [SMILES: c1ccc2c(c1)-c1ccccc1C21c2ccccc2-c2ccc(Nc3ccc4ccccc4c3)cc21] | Sub 2-141 |
| 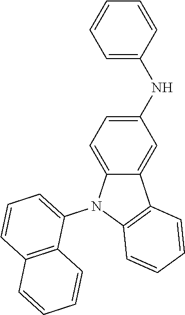 [SMILES: c1ccc(Nc2ccc3c(c2)c2ccccc2n3-c2cccc3ccccc23)cc1] | Sub 2-142 |
| 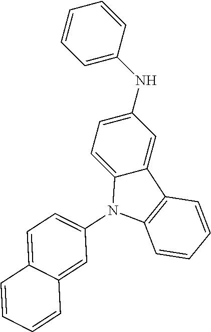 [SMILES: c1ccc(Nc2ccc3c(c2)c2ccccc2n3-c2ccc3ccccc3c2)cc1] | Sub 2-143 |
| 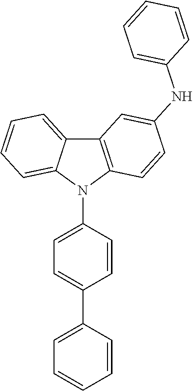 [SMILES: c1ccc(Nc2ccc3c(c2)c2ccccc2n3-c2ccc(-c3ccccc3)cc2)cc1] | Sub 2-144 |
| 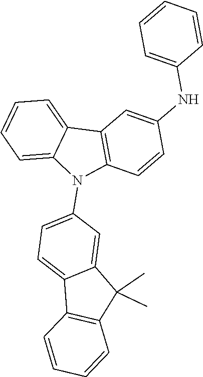 [SMILES: CC1(C)c2ccccc2-c2ccc(-n3c4ccccc4c4cc(Nc5ccccc5)ccc43)cc21] | Sub 2-145 |
| 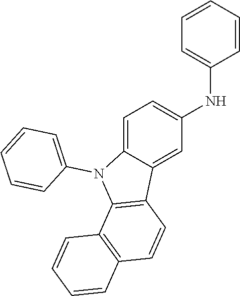 [SMILES: c1ccc(Nc2ccc3c(c2)c2ccc4ccccc4c2n3-c2ccccc2)cc1] | Sub 2-146 |
| 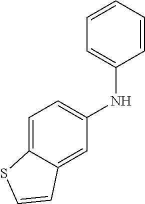 [SMILES: c1ccc(Nc2ccc3sccc3c2)cc1] | Sub 2-147 |
| 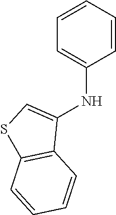 [SMILES: c1ccc(Nc2csc3ccccc23)cc1] | Sub 2-148 |
| 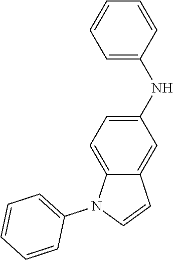 [SMILES: c1ccc(Nc2ccc3c(ccn3-c3ccccc3)c2)cc1] | Sub 2-149 |
| 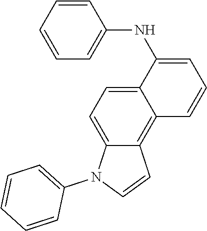 [SMILES: c1ccc(Nc2cccc3c2ccc2c3ccn2-c2ccccc2)cc1] | Sub 2-150 |
| 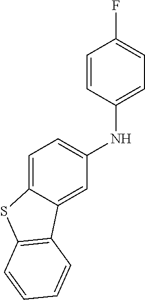 [SMILES: Fc1ccc(Nc2ccc3sc4ccccc4c3c2)cc1] | Sub 2-151 |
| 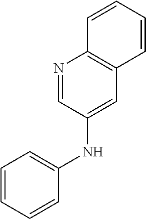 [SMILES: c1ccc(Nc2cnc3ccccc3c2)cc1] | Sub 2-152 |
| 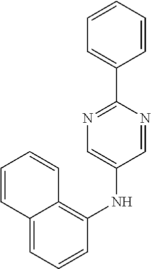 [SMILES: c1ccc(-c2ncc(Nc3cccc4ccccc34)cn2)cc1] | Sub 2-153 |
| 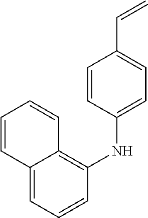 [SMILES: C=Cc1ccc(Nc2cccc3ccccc23)cc1] | Sub 2-154 |
| 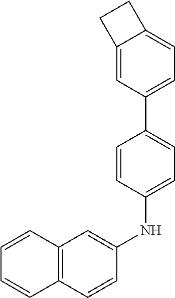 [SMILES: c1ccc2cc(Nc3ccc(-c4ccc5c(c4)CC5)cc3)ccc2c1] | Sub 2-155 |
| 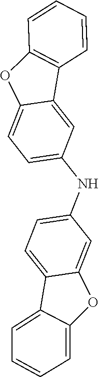 [SMILES: c1ccc2c(c1)oc1cc(Nc3ccc4oc5ccccc5c4c3)ccc12] | Sub 2-156 |
| 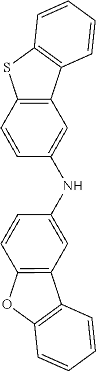 [SMILES: c1ccc2c(c1)oc1ccc(Nc3ccc4sc5ccccc5c4c3)cc12] | Sub 2-157 |
| 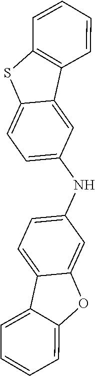 [SMILES: c1ccc2c(c1)oc1cc(Nc3ccc4sc5ccccc5c4c3)ccc12] | Sub 2-158 |
| 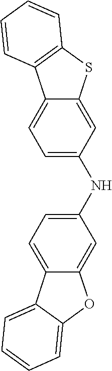 [SMILES: c1ccc2c(c1)oc1cc(Nc3ccc4c(c3)sc3ccccc34)ccc12] | Sub 2-159 |
| 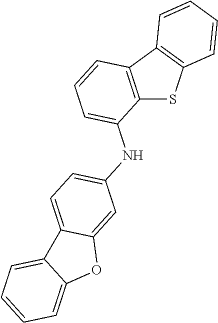 [SMILES: c1ccc2c(c1)oc1cc(Nc3cccc4c3sc3ccccc34)ccc12] | Sub 2-160 |
| 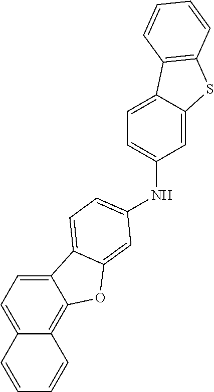 [SMILES: c1ccc2c(c1)ccc1c3ccc(Nc4ccc5c(c4)sc4ccccc45)cc3oc21] | Sub 2-161 |
| 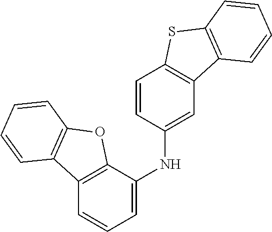 [SMILES: c1ccc2c(c1)oc1c(Nc3ccc4sc5ccccc5c4c3)cccc12] | Sub 2-162 |
| 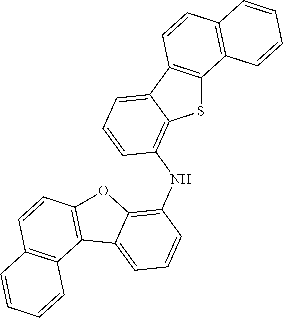 [SMILES: c1ccc2c(c1)ccc1c3cccc(Nc4cccc5c4oc4ccc6ccccc6c45)c3sc21] | Sub 2-163 |
| 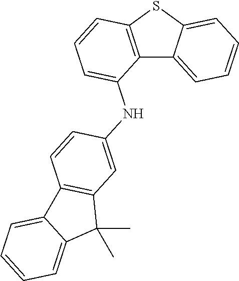 [SMILES: CC1(C)c2ccccc2-c2ccc(Nc3cccc4sc5ccccc5c34)cc21] | Sub 2-164 |
| 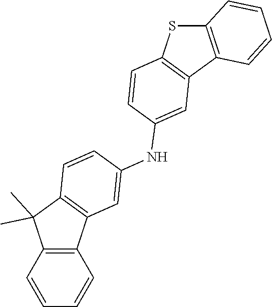 [SMILES: CC1(C)c2ccccc2-c2cc(Nc3ccc4sc5ccccc5c4c3)ccc21] | Sub 2-165 |
| 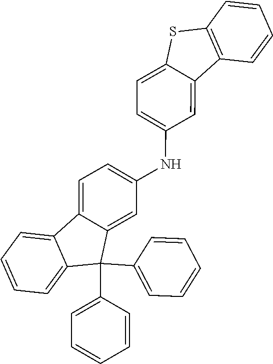 [SMILES: c1ccc(C2(c3ccccc3)c3ccccc3-c3ccc(Nc4ccc5sc6ccccc6c5c4)cc32)cc1] | Sub 2-166 |
| 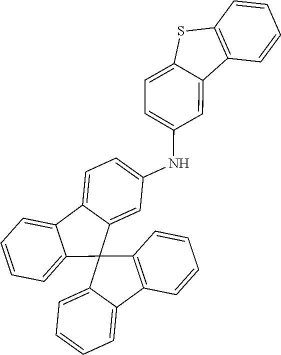 [SMILES: c1ccc2c(c1)-c1ccccc1C21c2ccccc2-c2ccc(Nc3ccc4sc5ccccc5c4c3)cc21] | Sub 2-167 |
| 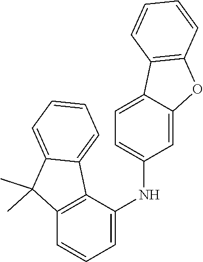 [SMILES: CC1(C)c2ccccc2-c2c(Nc3ccc4c(c3)oc3ccccc34)cccc21] | Sub 2-168 |
| 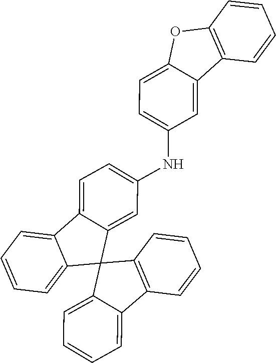 [SMILES: c1ccc2c(c1)-c1ccccc1C21c2ccccc2-c2ccc(Nc3ccc4oc5ccccc5c4c3)cc21] | Sub 2-169 |
| 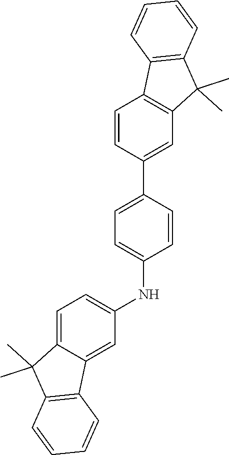 | Sub 2-170 |
| 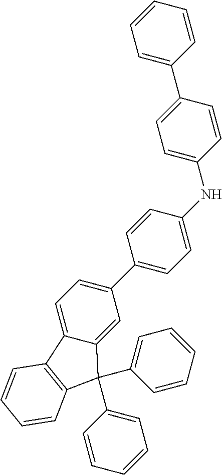 [SMILES: c1ccc(-c2ccc(Nc3ccc(-c4ccc5c(c4)C(c4ccccc4)(c4ccccc4)c4ccccc4-5)cc3)cc2)cc1] | Sub 2-171 |
| 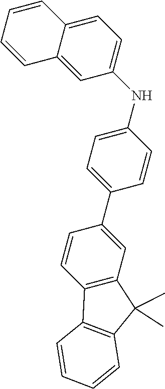 [SMILES: CC1(C)c2ccccc2-c2ccc(-c3ccc(Nc4ccc5ccccc5c4)cc3)cc21] | Sub 2-172 |
| 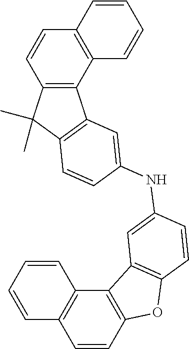 [SMILES: CC1(C)c2ccc(Nc3ccc4oc5ccc6ccccc6c5c4c3)cc2-c2c1ccc1ccccc21] | Sub 2-173 |
| 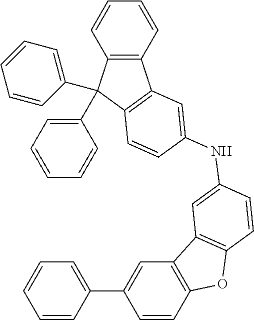 [SMILES: c1ccc(-c2ccc3oc4ccc(Nc5ccc6c(c5)-c5ccccc5C6(c5ccccc5)c5ccccc5)cc4c3c2)cc1] | Sub 2-174 |
| 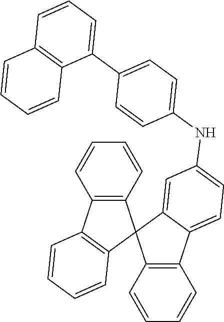 [SMILES: c1ccc2c(c1)-c1ccccc1C21c2ccccc2-c2ccc(Nc3ccc(-c4cccc5ccccc45)cc3)cc21] | Sub 2-175 |
| 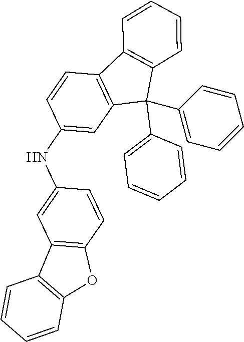 [SMILES: c1ccc(C2(c3ccccc3)c3ccccc3-c3ccc(Nc4ccc5oc6ccccc6c5c4)cc32)cc1] | Sub 2-176 |
| 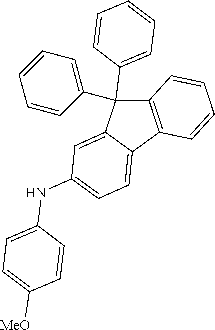 [SMILES: COc1ccc(Nc2ccc3c(c2)C(c2ccccc2)(c2ccccc2)c2ccccc2-3)cc1] | Sub 2-177 |
| 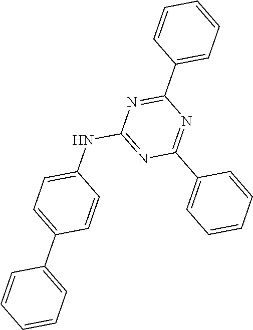 [SMILES: c1ccc(-c2ccc(Nc3nc(-c4ccccc4)nc(-c4ccccc4)n3)cc2)cc1] | Sub 2-178 |
| 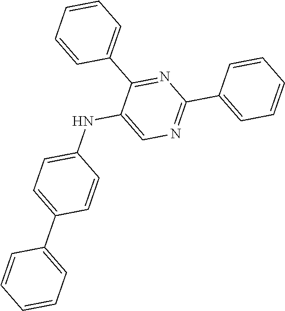 [SMILES: c1ccc(-c2ccc(Nc3cnc(-c4ccccc4)nc3-c3ccccc3)cc2)cc1] | Sub 2-179 |
| 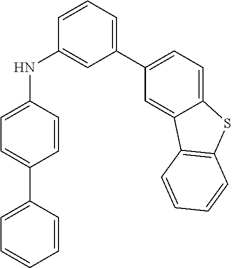 [SMILES: c1ccc(-c2ccc(Nc3cccc(-c4ccc5sc6ccccc6c5c4)c3)cc2)cc1] | Sub 2-180 |
| 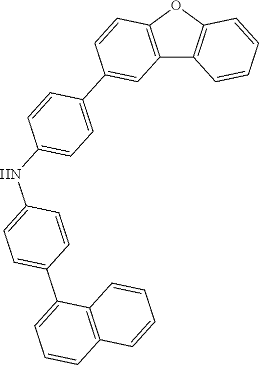 [SMILES: c1ccc2c(-c3ccc(Nc4ccc(-c5ccc6oc7ccccc7c6c5)cc4)cc3)cccc2c1] | Sub 2-181 |
| 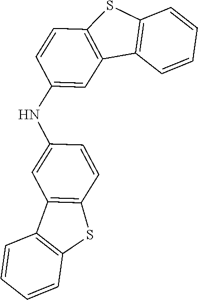 [SMILES: c1ccc2c(c1)sc1ccc(Nc3ccc4sc5ccccc5c4c3)cc12] | Sub 2-182 |
| 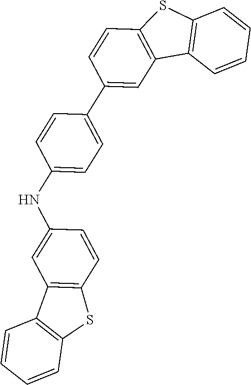 [SMILES: c1ccc2c(c1)sc1ccc(Nc3ccc(-c4ccc5sc6ccccc6c5c4)cc3)cc12] | Sub 2-183 |
| 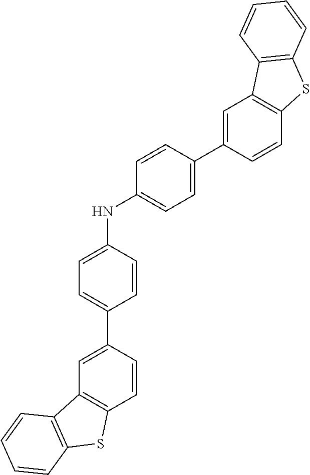 [SMILES: c1ccc2c(c1)sc1ccc(-c3ccc(Nc4ccc(-c5ccc6sc7ccccc7c6c5)cc4)cc3)cc12] | Sub 2-184 |
| 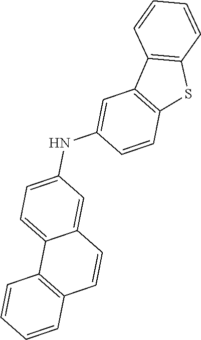 [SMILES: c1ccc2c(c1)ccc1cc(Nc3ccc4sc5ccccc5c4c3)ccc12] | Sub 2-185 |
| 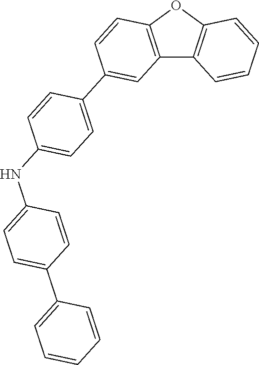 [SMILES: c1ccc(-c2ccc(Nc3ccc(-c4ccc5oc6ccccc6c5c4)cc3)cc2)cc1] | Sub 2-186 |
| 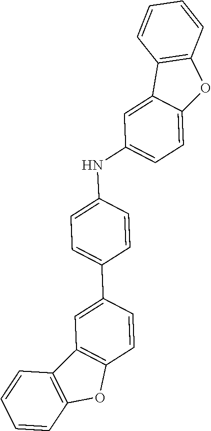 [SMILES: c1ccc2c(c1)oc1ccc(Nc3ccc(-c4ccc5oc6ccccc6c5c4)cc3)cc12] | Sub 2-187 |
| 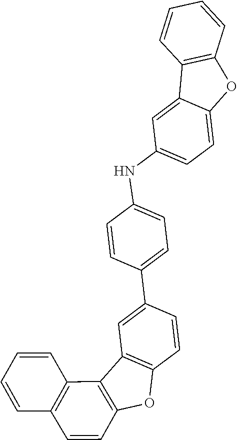 [SMILES: c1ccc2c(c1)ccc1oc3ccc(-c4ccc(Nc5ccc6oc7ccccc7c6c5)cc4)cc3c12] | Sub 2-188 |
| 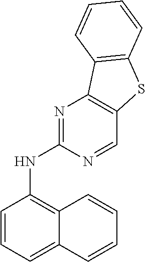 [SMILES: c1ccc2c(Nc3ncc4sc5ccccc5c4n3)cccc2c1] | Sub 2-189 |
| 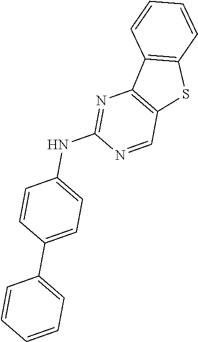 [SMILES: c1ccc(-c2ccc(Nc3ncc4sc5ccccc5c4n3)cc2)cc1] | Sub 2-190 |
| 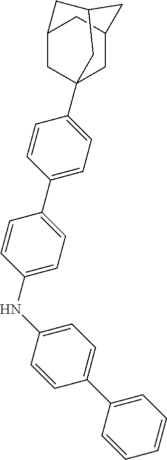 [SMILES: c1ccc(-c2ccc(Nc3ccc(-c4ccc(C56CC7CC(CC(C7)C5)C6)cc4)cc3)cc2)cc1] | Sub 2-191 |
| 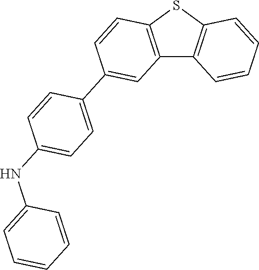 [SMILES: c1ccc(Nc2ccc(-c3ccc4sc5ccccc5c4c3)cc2)cc1] | Sub 2-192 |
| 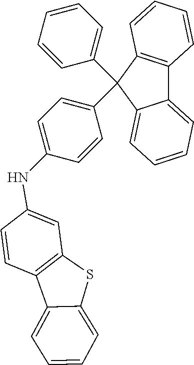 [SMILES: c1ccc(C2(c3ccc(Nc4ccc5c(c4)sc4ccccc45)cc3)c3ccccc3-c3ccccc32)cc1] | Sub 2-193 |
| 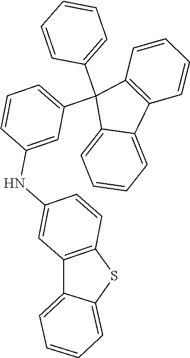 [SMILES: c1ccc(C2(c3cccc(Nc4ccc5sc6ccccc6c5c4)c3)c3ccccc3-c3ccccc32)cc1] | Sub 2-194 |
| 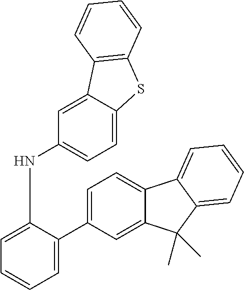 [SMILES: CC1(C)c2ccccc2-c2ccc(-c3ccccc3Nc3ccc4sc5ccccc5c4c3)cc21] | Sub 2-195 |
| 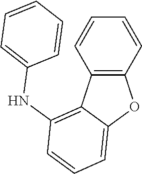 [SMILES: c1ccc(Nc2cccc3oc4ccccc4c23)cc1] | Sub 2-196 |
| 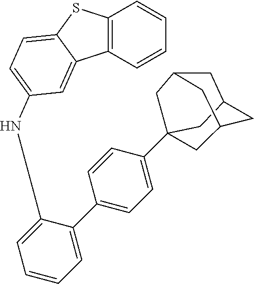 [SMILES: c1ccc(-c2ccc(C34CC5CC(CC(C5)C3)C4)cc2)c(Nc2ccc3sc4ccccc4c3c2)c1] | Sub 2-197 |
| 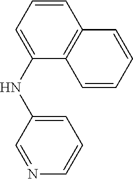 [SMILES: c1cncc(Nc2cccc3ccccc23)c1] | Sub 2-198 |
| 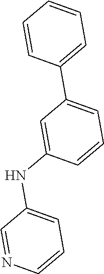 [SMILES: c1ccc(-c2cccc(Nc3cccnc3)c2)cc1] | Sub 2-199 |
| 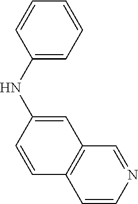 [SMILES: c1ccc(Nc2ccc3ccncc3c2)cc1] | Sub 2-200 |
| 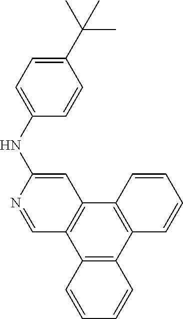 [SMILES: CC(C)(C)c1ccc(Nc2cc3c4ccccc4c4ccccc4c3cn2)cc1] | Sub 2-201 |
| 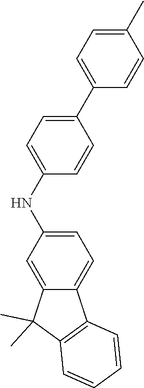 | Sub 2-202 |
| 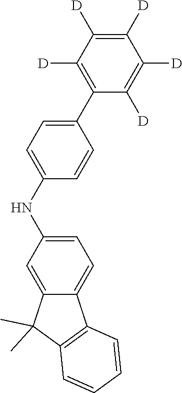 [SMILES: [2H]c1c([2H])c([2H])c(-c2ccc(Nc3ccc4c(c3)C(C)(C)c3ccccc3-4)cc2)c([2H])c1[2H]] | Sub 2-203 |
| 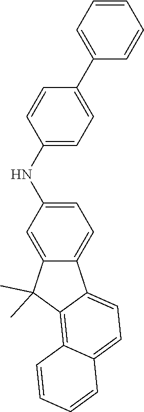 [SMILES: CC1(C)c2cc(Nc3ccc(-c4ccccc4)cc3)ccc2-c2ccc3ccccc3c21] | Sub 2-204 |
| 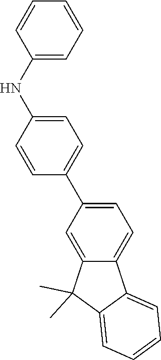 [SMILES: CC1(C)c2ccccc2-c2ccc(-c3ccc(Nc4ccccc4)cc3)cc21] | Sub 2-205 |
| 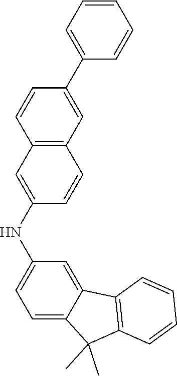 [SMILES: CC1(C)c2ccccc2-c2cc(Nc3ccc4cc(-c5ccccc5)ccc4c3)ccc21] | Sub 2-206 |
| 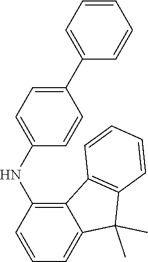 [SMILES: CC1(C)c2ccccc2-c2c(Nc3ccc(-c4ccccc4)cc3)cccc21] | Sub 2-207 |
| 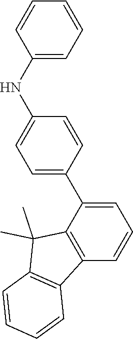 [SMILES: CC1(C)c2ccccc2-c2cccc(-c3ccc(Nc4ccccc4)cc3)c21] | Sub 2-208 |
| 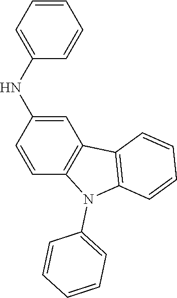 [SMILES: c1ccc(Nc2ccc3c(c2)c2ccccc2n3-c2ccccc2)cc1] | Sub 2-209 |
| 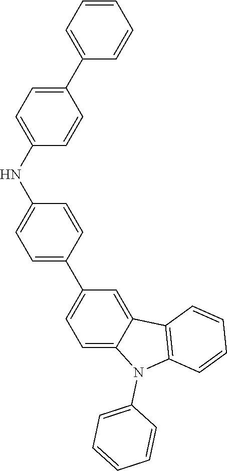 [SMILES: c1ccc(-c2ccc(Nc3ccc(-c4ccc5c(c4)c4ccccc4n5-c4ccccc4)cc3)cc2)cc1] | Sub 2-210 |
| 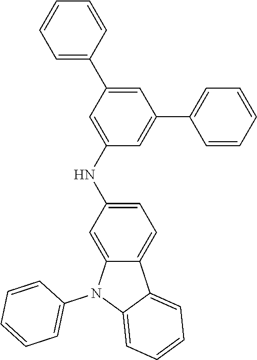 [SMILES: c1ccc(-c2cc(Nc3ccc4c5ccccc5n(-c5ccccc5)c4c3)cc(-c3ccccc3)c2)cc1] | Sub 2-211 |
| 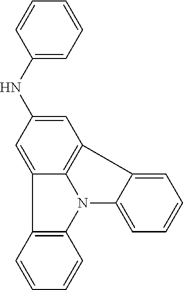 [SMILES: c1ccc(Nc2cc3c4ccccc4n4c5ccccc5c(c2)c34)cc1] | Sub 2-212 |
| 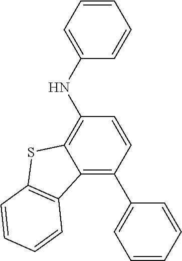 [SMILES: c1ccc(Nc2ccc(-c3ccccc3)c3c2sc2ccccc23)cc1] | Sub 2-213 |
| 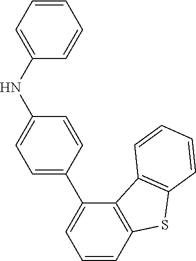 [SMILES: c1ccc(Nc2ccc(-c3cccc4sc5ccccc5c34)cc2)cc1] | Sub 2-214 |
| 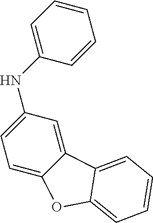 [SMILES: c1ccc(Nc2ccc3oc4ccccc4c3c2)cc1] | Sub 2-215 |
| 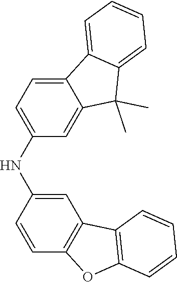 [SMILES: CC1(C)c2ccccc2-c2ccc(Nc3ccc4oc5ccccc5c4c3)cc21] | Sub 2-216 |
| 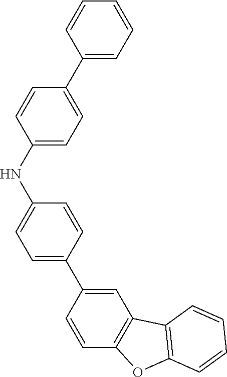 [SMILES: c1ccc(-c2ccc(Nc3ccc(-c4ccc5oc6ccccc6c5c4)cc3)cc2)cc1] | Sub 2-217 |
| 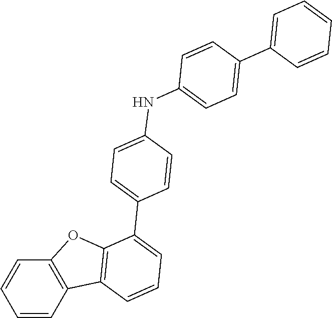 [SMILES: c1ccc(-c2ccc(Nc3ccc(-c4cccc5c4oc4ccccc45)cc3)cc2)cc1] | Sub 2-218 |
| 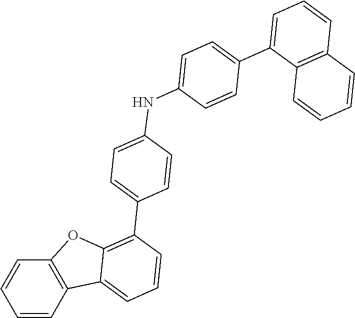 [SMILES: c1ccc2c(-c3ccc(Nc4ccc(-c5cccc6c5oc5ccccc56)cc4)cc3)cccc2c1] | Sub 2-219 |
| 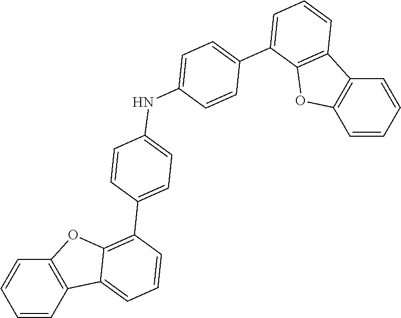 [SMILES: c1ccc2c(c1)oc1c(-c3ccc(Nc4ccc(-c5cccc6c5oc5ccccc56)cc4)cc3)cccc12] | Sub 2-220 |
| 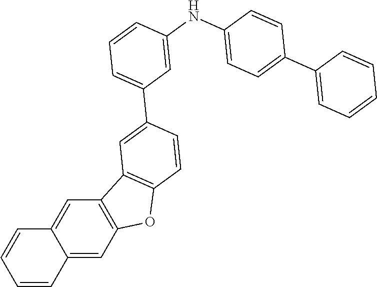 [SMILES: c1ccc(-c2ccc(Nc3cccc(-c4ccc5oc6cc7ccccc7cc6c5c4)c3)cc2)cc1] | Sub 2-221 |
| 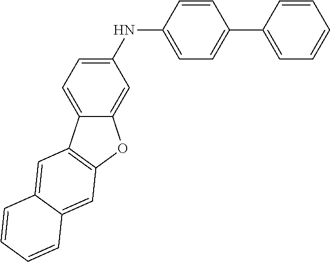 [SMILES: c1ccc(-c2ccc(Nc3ccc4c(c3)oc3cc5ccccc5cc34)cc2)cc1] | Sub 2-222 |
| 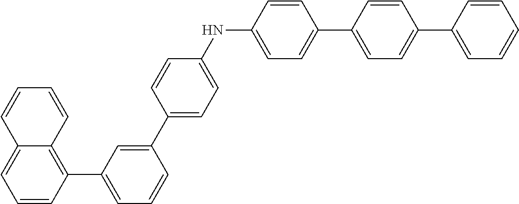 [SMILES: c1ccc(-c2ccc(-c3ccc(Nc4ccc(-c5cccc(-c6cccc7ccccc67)c5)cc4)cc3)cc2)cc1] | Sub 2-223 |

| Compound | FD-MS |
|---|---|
| Sub 2-1 | m/z = 169.09 (C₁₂H₁₁N = 169.22) |
| Sub 2-2 | m/z = 245.12 (C₁₈H₁₅N = 245.32) |
| Sub 2-3 | m/z = 245.12 (C₁₈H [symbol] N = 245.32) |
| Sub 2-4 | m/z = 321.15 (C₂₄H₁₉N = 321.41) |
| Sub 2-5 | m/z = 321.15 (C₂₄H₁₉N = 321.41) |
| Sub 2-6 | m/z = 269.12 (C₂₆H₁₅N = 269.34) |
| Sub 2-7 | m/z = 269.12 (C₃₀H₁₅N = 269.34) |
| Sub 2-8 | m/z = 295.14 (C₃₂H₁₇N = 295.38) |
| Sub 2-9 | m/z = 409.18 (C₃₁H₂₃N = 409.52) |
| Sub 2-10 | m/z = 483.20 (C₃₇H₂₅N = 483.60) |
| Sub 2-11 | m/z = 459.20 (C₃₅H₂₅N = 459.58) |
| Sub 2-12 | m/z = 485.21 (C₃₇H₂₇N = 485.62) |
| Sub 2-13 | m/z = 275.08 (C₁₈H₁₃NS = 275.37) |
| Sub 2-14 | m/z = 335.13 (C₂₄H₁₇NO = 335.40) |
| Sub 2-15 | m/z = 297.13 (C₂₀H₁₅N₃ = 297.35) |
| Sub 2-16 | m/z = 219.10 (C₁₆H₁₃N = 219.28) |
| Sub 2-17 | m/z = 249.12 (C₁₇H₁₅NO = 249.31) |
| Sub 2-18 | m/z = 197.12 (C₁₄H₁₅N = 197.28) |
| Sub 2-19 | m/z = 229.11 (C₁₄H₁₅NO₂ = 229.27) |
| Sub 2-20 | m/z = 174.12 (C₁₂H₆D₅N = 174.25) |
| Sub 2-21 | m/z = 281.21 (C₂₀H₂₇N = 281.44) |
| Sub 2-22 | m/z = 321.15 (C₂₄H₁₉N = 321.41) |
| Sub 2-23 | m/z = 321.15 (C₂₄H₁₉N = 321.41) |
| Sub 2-24 | m/z = 321.15 (C₂₄H₁₉N = 321.41) |
| Sub 2-25 | m/z = 321.15 (C₂₄H₁₉N = 321.41) |
| Sub 2-26 | m/z = 321.15 (C₂₄H₁₉N = 321.41) |
| Sub 2-27 | m/z = 297.13 (C₂₀H₁₅N₃ = 297.35) |
| Sub 2-28 | m/z = 499.20 (C₃₆H₂₅N₃ = 499.60) |
| Sub 2-29 | m/z = 499.20 (C₃₆H₂₂N₂ = 410.51) |
| Sub 2-30 | m/z = 424.16 (C₃₀H₂₀N₂O = 424.49) |
| Sub 2-31 | m/z = 440.13 (C₃₀H₂₀N₂S = 440.56) |
| Sub 2-32 | m/z = 384.16 (C₂₈H₂₀N₂ = 384.47) |
| Sub 2-33 | m/z = 334.15 (C₂₄H₁₈N₂ = 334.41) |
| Sub 2-34 | m/z = 450.21 (C₃₃H₂₆N₂ = 450.57) |
| Sub 2-35 | m/z = 410.18 (C₃₀H₂₂N₂ = 410.51) |
| Sub 2-36 | m/z = 410.18 (C₃₀H₂₂N₂ = 410.51) |
| Sub 2-37 | m/z = m/z 575.24 (C₄₂H₂₉N₃ = 575.70) |
| Sub 2-38 | m/z = 574.24 (C₄₃H₃₀N₂ = 574.71) |
| Sub 2-39 | m/z = 460.19 (C₃₄H₂₄N₂ = 460.57) |
| Sub 2-40 | m/z = 460.19 (C₃₄H₂₄N₂ = 460.57) |
| Sub 2-41 | m/z = 461.19 (C₃₃H₂₃N₃ = 461.56) |
| Sub 2-42 | m/z = 626.27 (C₄₇H₃₄N₂ = 626.79) |
| Sub 2-43 | m/z = 565.23 (C₃₉H₂₇N₅ = 565.67) |
| Sub 2-44 | m/z = 415.21 (C₃₀H₁₇D₅N₂ = 415.54) |
| Sub 2-45 | m/z = 486.21 (C₃₆H₂₆N₂ = 486.61) |
| Sub 2-46 | m/z = 415.21 (C₃₀H₁₇D₅N₂ = 415.54) |
| Sub 2-47 | m/z = 225.15 (C₁₆H₁₉N = 225.33) |
| Sub 2-48 | m/z = 220.10 (C₁₅H₁₂N₂ = 220.27) |
| Sub 2-49 | m/z = 220.10 (C₁₅H₁₂N₂ = 220.27) |
| Sub 2-50 | m/z = 269.12 (C₂₀H₁₅N = 269.34) |
| Sub 2-51 | m/z = 269.12 (C₂₀H₁₅N = 269.34) |
| Sub 2-52 | m/z = 293.12 (C₂₂H₁₅N = 293.36) |
| Sub 2-53 | m/z = 250.15 (C₁₈H₁₀D₅N = 250.35) |
| Sub 2-54 | m/z = 250.15 (C₁₈H₁₀D₅N = 250.35) |
| Sub 2-55 | m/z = 246.12 (C₁₇H₁₄N₂ = 246.31) |
| Sub 2-56 | m/z = 295.14 (C₂₂H₁₇N = 295.38) |
| Sub 2-57 | m/z = 295.14 (C₂₂H₁₇N = 295.38) |
| Sub 2-58 | m/z = 295.14 (C₂₂H₁₇N = 295.38) |
| Sub 2-59 | m/z = 300.17 (C₂₃H [symbol] D₅N = 300.41) |
| Sub 2-60 | m/z = 295.14 (C₂₂H₁₇N = 295.38) |
| Sub 2-61 | m/z = 295.14 (C₂₂H₁₇N = 295.38) |
| Sub 2-62 | m/z = 269.12 (C₂₀H₁₅N = 269.34) |
| Sub 2-63 | m/z = 345.15 (C₂₆H₁₉N = 345.44) |
| Sub 2-64 | m/z = 296.13 (C₂₁H₁₆N₂ = 296.37) |
| Sub 2-65 | m/z = 346.15 (C₂₅H₁₉N₂ = 346.42) |
| Sub 2-66 | m/z = 321.15 (C₂₄H₁₉N = 321.41) |
| Sub 2-67 | m/z = 321.15 (C₂₄H₁₉N = 321.41) |
| Sub 2-68 | m/z = 421.18 (C₃₂H₂₃N = 421.53) |
| Sub 2-69 | m/z = 300.17 (C₂₂H₁₂D₅N = 300.41) |
| Sub 2-70 | m/z = 421.18 (C₃₂H₂₃N = 421.53) |
| Sub 2-71 | m/z = 321.15 (C₂₄H₁₉N = 321.41) |
| Sub 2-72 | m/z = 371.17 (C₂₈H₂₁N = 371.47) |
| Sub 2-73 | m/z = 319.14 (C₂₄H₁₇N = 319.40) |
| Sub 2-74 | m/z = 293.12 (C₂₂H₁₅N = 293.36) |
| Sub 2-75 | m/z = 395.17 (C₃₀H₂₁N = 395.49) |
| Sub 2-76 | m/z = 386.18 (C₂₈H₂₂N₂ = 386.49) |
| Sub 2-77 | m/z = 244.14 (C₁₆H₈D₅N = 224.31) |
| Sub 2-78 | m/z = 275.08 (C₁₈H₁₃NS = 275.37) |
| Sub 2-79 | m/z = 325.09 (C₂₂H₁₅NS = 325.43) |
| Sub 2-80 | m/z = 325.09 (C₂₂H₁₅NS = 325.43) |
| Sub 2-81 | m/z = 351.11 (C₂₄H₁₇NS = 351.46) |
| Sub 2-82 | m/z = 326.09 (C₂₁H₁₄N₂S = 326.41) |
| Sub 2-83 | m/z = 351.11 (C₂₄H₁₇NS = 351.46) |
| Sub 2-84 | m/z = 275.08 (C₁₈H₁₃NS = 275.37) |
| Sub 2-85 | m/z = 290.09 (C₁₈H₁₄N₂S = 290.38) |
| Sub 2-86 | m/z = 325.09 (C₂₂H₁₅NS = 325.43) |
| Sub 2-87 | m/z = 351.11 (C₂₁H₁₇NS = 351.46) |
| Sub 2-88 | m/z = 381.06 (C₂₄H₁₅NS₂ = 385.51) |
| Sub 2-89 | m/z = 401.12 (C₂₈H₁₉NS = 401.52) |
| Sub 2-90 | m/z = 275.08 (C₁₈H₁₅NS = 275.37) |
| Sub 2-91 | m/z = 351.11 (C₂₄H₁₇NS = 351.46) |
| Sub 2-92 | m/z = 325.09 (C₂₂H₁₅NS = 325.43) |
| Sub 2-93 | m/z = 401.12 (C₂₈H₁₃NS = 410.52) |
| Sub 2-94 | m/z = 351.11 (C₂₄H₁₂NS = 351.46) |
| Sub 2-95 | m/z = 352.10 (C₂₃H₁₆N₂S = 352.45) |
| Sub 2-96 | m/z = 440.13 (C₃₀H [symbol] N₂S = 440.56) |
| Sub 2-97 | m/z = 375.11 (C₂₆H₁₇NS = 375.48) |
| Sub 2-98 | m/z = 381.06 (C₂₄H₁₅NS₂ = 381.51) |
| Sub 2-99 | m/z = 351.11 (C₂₄H₁₇NS = 351.46) |
| Sub 2-100 | m/z = 309.12 (C₂₂H₁₅NO = 309.36) |
| Sub 2-101 | m/z = 259.10 (C₁₈H₁₃NO = 259.30) |
| Sub 2-102 | m/z = 309.12 (C₂₂H₁₅NO = 309.36) |
| Sub 2-103 | m/z = 335.13 (C₂₄H₁₇NO = 3354.0) |
| Sub 2-104 | m/z = 349.11 (C₂₄H₁₅NO₂ = 349.38) |
| Sub 2-105 | m/z = 309.12 (C₂₂H₁₅NO = 309.36) |
| Sub 2-106 | m/z = 259.10 (C₁₈H₁₃NO = 259.30) |
| Sub 2-107 | m/z = 335.13 (C₂₄H₁₇NO = 335.40) |
| Sub 2-108 | m/z = 259.10 (C₁₈H₁₃NO = 359.30) |
| Sub 2-109 | m/z = 335.13 (C₂₄H₁₇NO = 335.40) |
| Sub 2-110 | m/z = 461.18 (C₃₄H₂₃NO = 461.55) |
| Sub 2-111 | m/z = 335.13 (C₂₄H₁₇NO = 335.40) |
| Sub 2-112 | m/z = 335.13 (C₂₄H₁₇NO = 335.40) |
| Sub 2-113 | m/z = 335.13 (C₂₄H₁₇NO = 335.40) |
| Sub 2-114 | m/z = 385.15 (C₂₈H₁₉NO = 385.46) |
| Sub 2-115 | m/z = 411.16 (C₃₀H₂₁NO = 411.49) |
| Sub 2-116 | m/z = 411.16 (C₃₀H₂₁NO = 411.49) |
| Sub 2-117 | m/z = 285.15 (C₂₁H₁₉N = 285.38) |
| Sub 2-118 | m/z = 290.18 (C₂₁H₁₄D₃N = 290.41) |
| Sub 2-119 | m/z = 335.17 (C₂₅H₂₁N = 335.44) |
| Sub 2-120 | m/z = 361.18 (C₂₇H₂₃N = 361.48) |
| Sub 2-121 | m/z = 391.14 (C₂₇H₂₁NS = 391.53) |
| Sub 2-122 | m/z = 401.21 (C₃₀H₂₇N = 401.54) |
| Sub 2-123 | m/z = 335.17 (C₂₅H₂₁N = 335.44) |
| Sub 2-124 | m/z = 335.17 (C₂₅H₂₁N = 335.44) |
| Sub 2-125 | m/z = 385.18 (C₂₉H₂₃N = 385.50) |
| Sub 2-126 | m/z = 361.18 (C₂₇H₂₃N = 361.48) |
| Sub 2-127 | m/z = 299.17 (C [symbol] H₂₁N = 299.41) |
| Sub 2-128 | m/z = 385.18 (C₂₉H₂₃N = 385.50) |
| Sub 2-129 | m/z = 409.18 (C₃₁H₂₃N = 409.52) |
| Sub 2-130 | m/z = 525.25 (C₄₀H₃₁N = 525.68) |
| Sub 2-131 | m/z = 409.18 (C₃₁H₂₃N = 409.52) |
| Sub 2-132 | m/z = 423.20 (C₃₂H₂₅N = 423.55) |
| Sub 2-133 | m/z = 439.19 (C₃₂H₂₅NO = 439.55) |
| Sub 2-134 | m/z = 459.20 (C₃₅H₂₅N = 459.58) |
| Sub 2-135 | m/z = 485.21 (C₃₇H₂₇N = 485.62) |
| Sub 2-136 | m/z = 562.24 (C₄₂H₃₀N₂ = 562.70) |
| Sub 2-137 | m/z = 485.12 (C₃₇H₂₇N = 485.62) |
| Sub 2-138 | m/z = 523.23 (C₄₀H₂₉N = 523.66) |
| Sub 2-139 | m/z = 407.17 (C₃₁H₂₃N = 407.51) |
| Sub 2-140 | m/z = 407.17 (C₃₁H₂₁N = 407.51) |
| Sub 2-141 | m/z = 483.20 (C₃₇H₂₅N = 483.60) |
| Sub 2-142 | m/z = 457.18 (C₃₅H₂₃N = 457.56) |
| Sub 2-143 | m/z = 410.18 (C₃₀H₂₂N₂ = 410.51) |
| Sub 2-144 | m/z = 384.16 (C₂₈H₂₀N₂ = 384.47) |
| Sub 2-145 | m/z = 384.16 (C₂₈H₂₀N₂ = 384.47) |
| Sub 2-146 | m/z = 410.18 (C₃₀H₂₂N₂ = 410.51) |
| Sub 2-147 | m/z = 450.21 (C₃₃H₂₆N₂ = 450.57) |
| Sub 2-148 | m/z = 384.16 (C₂₆H₂₀N₂ = 384.47) |
| Sub 2-149 | m/z = 225.06 (C₁₄H₁₁NS = 225.31) |
| Sub 2-150 | m/z = 225.06 (C₁₄H₁₁NS = 225.31) |
| Sub 2-151 | m/z = 284.13 (C₂₀H₁₅N₂ = 285.35) |
| Sub 2-152 | m/z = 334.15 (C₂₄H₁₆N₂ = 334.41) |
| Sub 2-153 | m/z = 293.07 (C₁₈H₁₂FNS = 293.36) |
| Sub 2-154 | m/z = 200.10 (C₁₅H₁₂N₂ = 220.27) |
| Sub 2-155 | m/z = 297.13 (C₂₉H₁₅N₃ = 297.35) |
| Sub 2-156 | m/z = 245.12 (C₁₈H₁₅N = 245.32) |
| Sub 2-157 | m/z = 321.15 (C₂₄H₁₉N = 321.41) |
| Sub 2-158 | m/z = 349.11 (C₂₄H₁₅NO₂ = 349.38) |
| Sub 2-159 | m/z = 365.09 (C₂₄H₁₅NOS = 365.45) |
| Sub 2-160 | m/z = 365.09 (C₂₄H₁₅NOS = 365.45) |
| Sub 2-161 | m/z = 365.09 (C₂₄H₁₅NOS = 365.45) |
| Sub 2-162 | m/z = 365.09 (C₂₄H₁₅NOS = 365.45) |
| Sub 2-163 | m/z = 415.10 (C₂₈H₁₇NOS = 415.51) |
| Sub 2-164 | m/z = 365.09 (C₂₄H₁₅NOS = 365.45) |
| Sub 2-165 | m/z = 465.12 (C₃₂H₁₉NOS = 465.57) |
| Sub 2-166 | m/z = 391.14 (C₂₇H₂₁NS = 391.53) |
| Sub 2-167 | m/z = 391.14 (C₂₇H₂₁NS = 391.53) |
| Sub 2-168 | m/z = 515.17 (C₃₇H₂₅NS = 515.67) |
| Sub 2-169 | m/z = 513.16 (C₃₇H₂₃NS = 513.65) |
| Sub 2-170 | m/z = 375.16 (C₂₇H₂₁NO = 375.46) |
| Sub 2-171 | m/z = 497.18 (C₃₇H₂₃NO = 497.58) |
| Sub 2-172 | m/z = 477.25 (C₃₆H₃₁N = 477.64) |
| Sub 2-173 | m/z = 561.25 (C₄₃H₃₁N = 561.73) |
| Sub 2-174 | m/z = 411.2 (C₃₁H₂₅N = 411.55) |
| Sub 2-175 | m/z = 475.19 (C₃₅H₂₃NO = 475.59) |
| Sub 2-176 | m/z = 575.22 (C [symbol] H₂₉NO = 575.71) |
| Sub 2-177 | m/z = 533.21 (C₄₁H₂₇N = 533.67) |
| Sub 2-178 | m/z = 499.19 (C₃₇H₂₅NO = 499.61) |
| Sub 2-179 | m/z = 439.19 (C₃₂H₂₃NO = 439.56) |
| Sub 2-180 | m/z = 400.17 (C₂₇H₂₀N₄ = 400.49) |
| Sub 2-181 | m/z = 399.17 (C₂₈H₂₁N₃ = 399.5) |
| Sub 2-182 | m/z = 427.14 (C₃₀H₂₁NS = 427.57) |
| Sub 2-183 | m/z = 461.18 (C₃₄H₂₃NO = 461.56) |
| Sub 2-184 | m/z = 381.06 (C₂₄H₁₅NS₂ = 381.51) |
| Sub 2-185 | m/z = 457.1 (C₃₀H₁₉NS₂ = 457.61) |
| Sub 2-186 | m/z = 533.13 (C₃₆H₂₃NS₂ = 533.71) |
| Sub 2-187 | m/z = 375.11 (C₂₅H₁₇NS = 375.49) |
| Sub 2-188 | m/z = 411.16 (C₃₀H₂₁NO = 411.5) |
| Sub 2-189 | m/z = 425.14 (C₃₀H₁₉NO₂ = 425.49) |
| Sub 2-190 | m/z = 475.16 (C₃₄H₂₁NO₂ = 475.55) |
| Sub 2-191 | m/z = 327.08 (C₂₀H₁₃N₃S = 327.41) |
| Sub 2-192 | m/z = 353.1 (C₂₂H₁₅N₃S = 353.44) |
| Sub 2-193 | m/z = 455.26 (C₃₄H₃₃N = 455.65) |
| Sub 2-194 | m/z = 351.11 (C₂₄H₁₇NS = 351.47) |
| Sub 2-195 | m/z = 515.17 (C₃₇H₂₅NS = 515.67) |
| Sub 2-196 | m/z = 515.17 (C₃₇H₂₅NS = 515.67) |
| Sub 2-197 | m/z = 467.17 (C₃₃H₂₅NS = 467.63) |
| Sub 2-198 | m/z = 259.1 (C₁₈H₁₃NO = 259.31) |
| Sub 2-199 | m/z = 485.22 (C₃₄H₃₁NS = 485.69) |
| Sub 2-200 | m/z = 220.10 (C₁₅H₁₂N₂ = 220.28) |
| Sub 2-201 | m/z = 246.12 (C₁₇H₁₄N₂ = 246.31) |
| Sub 2-202 | m/z = 220.10 (C₁₅H₁₂N₂ = 220.28) |
| Sub 2-203 | m/z = 376.19 (C₂₇H₂₄N₂ = 376.50) |
| Sub 2-204 | m/z = 375.20 (C₂₈H₂₅N = 375.52) |
| Sub 2-205 | m/z = 366.21 (C₂₇H₁₈D₅N = 366.52) |
| Sub 2-206 | m/z = 411.20 (C₃₁H₂₅N = 411.55) |
| Sub 2-207 | m/z = 361.18 (C₂₇H₂₃N = 361.49) |
| Sub 2-208 | m/z = 411.20 (C₃₁H₂₅N = 411.55) |
| Sub 2-209 | m/z = 361.18 (C₂₇H₂₃N = 361.49) |
| Sub 2-210 | m/z = 361.18 (C₂₇H₂₃N = 361.49) |
| Sub 2-211 | m/z = 334.15 (C₂₄H₁₈N₂ = 334.42) |
| Sub 2-212 | m/z = 486.12 (C₃₆H₂₅N₂ = 486.62) |
| Sub 2-213 | m/z = 486.21 (C₃₅H₂₆N₂ = 486.62) |
| Sub 2-214 | m/z = 332.13 (C₂₄H₁₆N₂ = 332.41) |
| Sub 2-215 | m/z = 351.11 (C₂₄H₁₇NS = 351.47) |
| Sub 2-216 | m/z = 351.11 (C₂₄H₁₇NS = 351.47) |
| Sub 2-217 | m/z = 259.10 (C₁₈H₁₃NO = 259.31) |
| Sub 2-218 | m/z = 375.16 (C₂₇H₂₁NO = 375.47) |
| Sub 2-219 | m/z = 411.16 (C₃₀H₂₁NO = 411.50) |
| Sub 2-220 | m/z = 411.16 (C₃₀H₂₁NO = 411.50) |
| Sub 2-221 | m/z = 461.18 (C₃₄H₂₃NO = 461.56) |
| Sub 2-222 | m/z = 385.15 (C₂₈H₁₉NO = 385.47) |
| Sub 2-223 | m/z = 523.23 (C40H29N = 523.68) |

[symbol]

indicates data missing or illegible when filed

Illustration of Synthesis of Final Product 1

Synthesis of 1-54

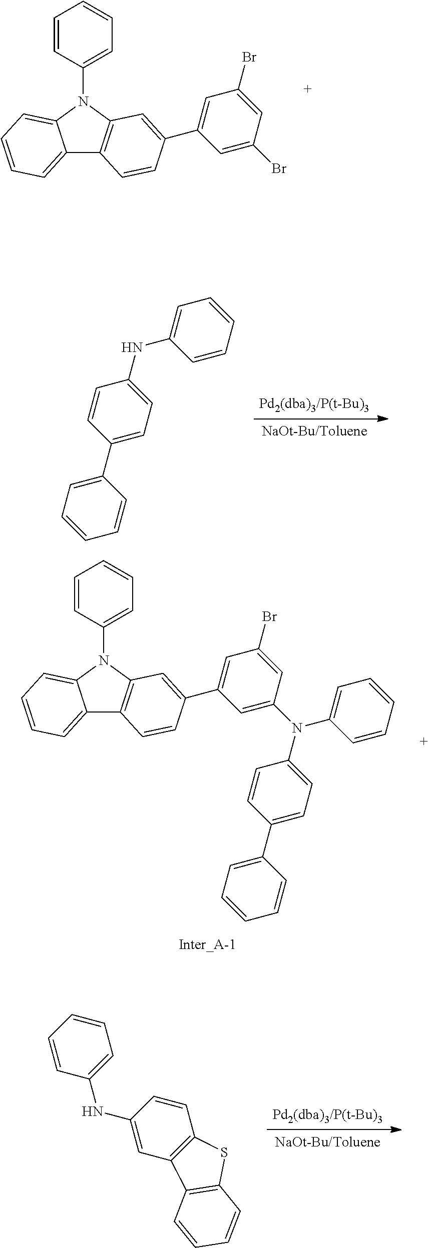

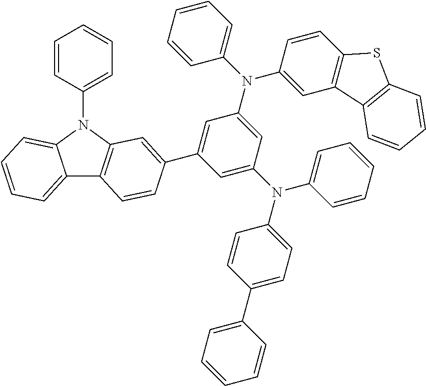 [SMILES: c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1]

1) Synthesis of Inter_A-1

N-phenyl-[1,1′-biphenyl]-4-amine (11.6 g, 47.3 mmol), toluene (500 ml), 2-(3,5-dibromophenyl)-9-phenyl-9H-carbazole (24.8 g, 52.0 mmol), Pd₂(dba)₃ (2.4 g, 2.6 mmol), P(t-Bu)₃ (1.05 g, 5.2 mmol), and NaOt-Bu (13.6 g, 141.8 mmol) were added, followed by stirring at 100° C. When the reaction was completed, extraction was performed with CH₂Cl₂ and water, and then an organic layer was dried with MgSO₄ and concentrated. The resultant organic matter was subjected to silica gel column chromatography and recrystallization to create Inter_A-1 22.8 g (yield: 75%).

2) Synthesis of 1-54

N-phenyldibenzo[b,d]thiophen-2-amine (8 g, 29.05 mmol), Inter_A-1 (20.5 g, 32 mmol), toluene (305 ml), Pd₂(dba)₃ (1.5 g, 1.6 mmol), P(t-Bu)₃ (0.65 g, 3.2 mmol), and NaOt-Bu (8.4 g, 87.2 mmol) were subjected to the same experimental method as in Inter_A-1, thereby creating product 1-54 18 g (yield: 74%).

Synthesis of 2-9

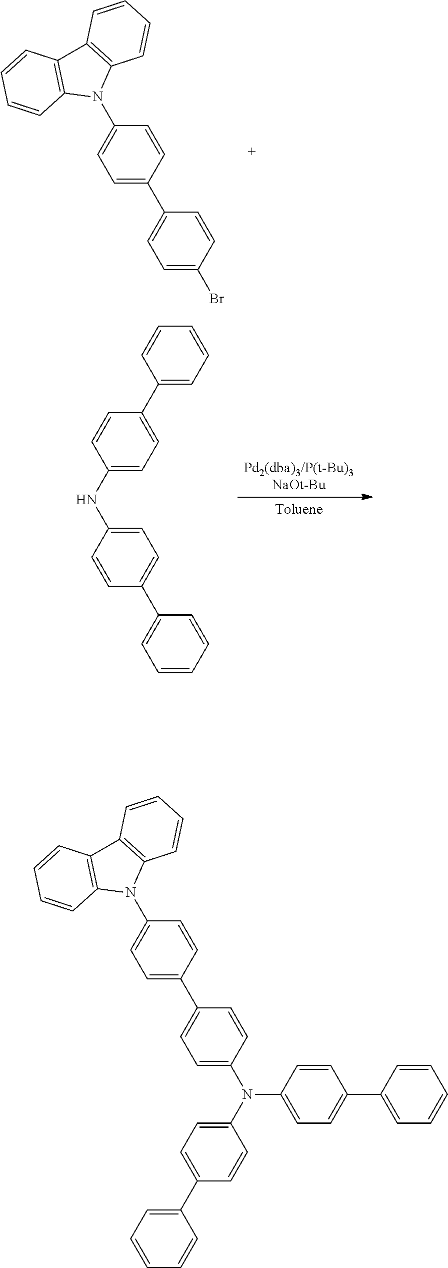

After Sub 2-26 (7 g, 21.8 mmol) was dissolved with toluene (230 ml) in a round bottom flask, Sub 1-2 (9.54 g, 24 mmol), Pd₂ (dba)₃ (1 g, 1.1 mmol), 50% P(t-Bu)₃ (1.1 ml, 2.2 mmol), and NaOt-Bu (6.91 g, 71.9 mmol) were added, followed by stirring at 100° C. When the reaction was completed, extraction was performed with CH₂Cl₂ and water, and then an organic layer was dried with MgSO₄ and concentrated. A resultant compound was subjected to silica gel column chromatography and recrystallization, thereby creating a product 11.69 g (yield: 84%).

Synthesis of 3-52

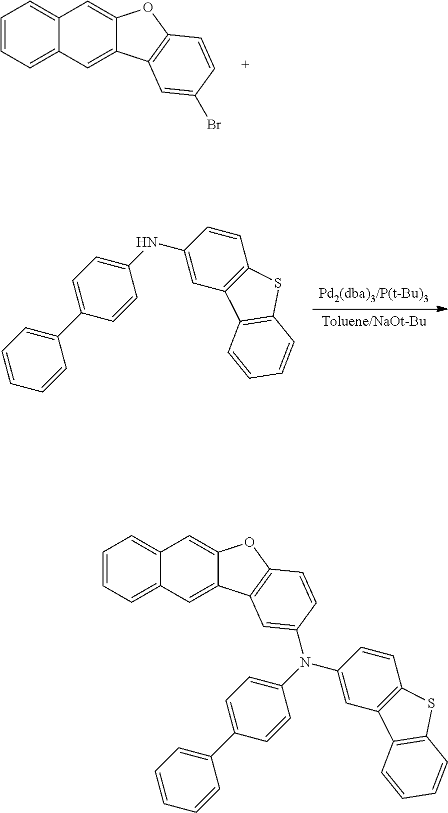 [SMILES: Brc1ccc2oc3cc4ccccc4cc3c2c1 ; c1ccc(-c2ccc(Nc3ccc4sc5ccccc5c4c3)cc2)cc1 ; "Pd2(dba)3/P(t-Bu)3" ; "Toluene/NaOt-Bu" ; c1ccc(-c2ccc(N(c3ccc4oc5cc6ccccc6cc5c4c3)c3ccc4sc5ccccc5c4c3)cc2)cc1]

2-bromonaphtho[2,3-b]benzofuran (10 g, 33.65 mmol), N-([1,1′-biphenyl]-4-yl)dibenzo[b,d]thiophen-2-amine (11.83 g, 33.65 mmol), Pd₂(dba)₃ (0.92 g, 1.01 mmol), P(t-Bu)₃ (6.81 g, 33.65 mmol), NaOt-Bu (6.47 g, 67.31 mmol), and toluene (337 ml) were subjected to the same experimental method as in Synthesis 2-9, thereby creating a product 15.28 g (yield: 80%).

Synthesis of 6-12

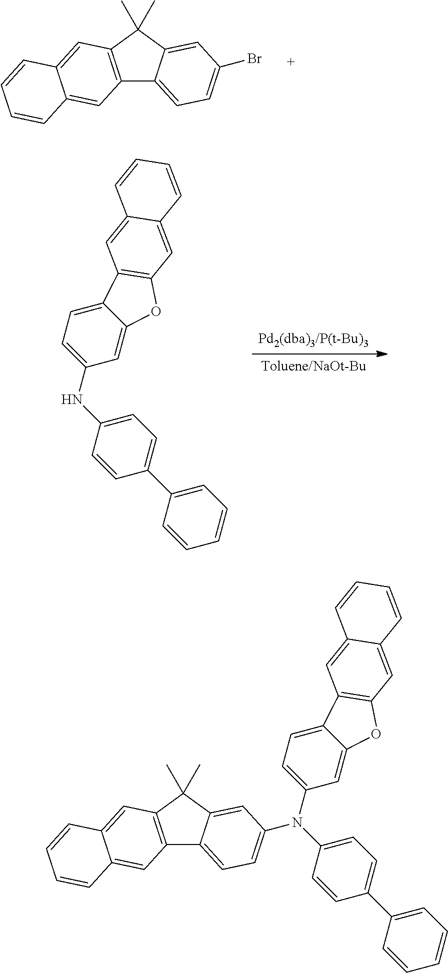 [SMILES: CC1(C)c2cc(Br)ccc2-c2cc3ccccc3cc21 ; c1ccc(-c2ccc(Nc3ccc4c(c3)oc3cc5ccccc5cc34)cc2)cc1 ; "Pd2(dba)3/P(t-Bu)3" ; "Toluene/NaOt-Bu" ; CC1(C)c2cc(N(c3ccc(-c4ccccc4)cc3)c3ccc4c(c3)oc3cc5ccccc5cc34)ccc2-c2cc3ccccc3cc21]

2-bromo-11,11-dimethyl-11H-benzo[b]fluorene (10 g, 30.94 mmol), N-([1,1′-biphenyl]-4-yl) naphtho[2,3-b]benzofuran-3-amine (11.93 g, 30.94 mmol), Pd₂(dba)₃ (0.85 g, 0.93 mmol), P(t-Bu)₃ (6.26 g, 30.94 mmol), NaOt-Bu (5.95 g, 61.88 mmol), and toluene (309 ml) were subjected to the same experimental method as in Synthesis 2-9, thereby creating a product 15.15 g (yield: 78%).

Synthesis of 11-4

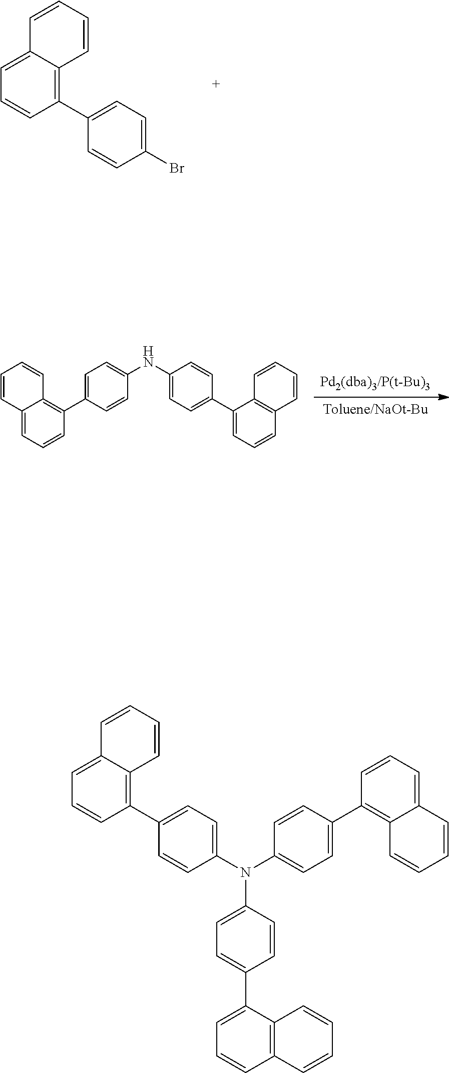 [SMILES: Brc1ccc(-c2cccc3ccccc23)cc1 ; c1ccc2c(-c3ccc(Nc4ccc(-c5cccc6ccccc56)cc4)cc3)cccc2c1 ; "Pd2(dba)3/P(t-Bu)3" ; "Toluene/NaOt-Bu" ; c1ccc2c(-c3ccc(N(c4ccc(-c5cccc6ccccc56)cc4)c4ccc(-c5cccc6ccccc56)cc4)cc3)cccc2c1]

1-(4-bromophenyl)naphthalene (10 g, 35.3 mmol), bis(4-(naphthalen-1-yl)phenyl)amine (14.8 g, 35.31 mmol), Pd₂(dba)₃ (0.97 g, 1.06 mmol), P(t-Bu)₃ (7.14 g, 35.31 mmol), NaOt-Bu (6.79 g, 70.63 mmol), and toluene (353 ml) were subjected to the same experimental method as in Synthesis 2-9, thereby creating a product 16.9 g (yield: 78%).

TABLE 3

| Compound | FD-MS | Compound | FD-MS |
|---|---|---|---|
| 1-1 | m/z = 562.24(C₄₂H₃₀N₂ = 562.72) | 1-2 | m/z = 602.27(C₄₃H₃₄N₂ = 602.78) |
| 1-3 | m/z = 563.24(C₄₁H₂₉N [symbol] = 563.70) | 1-4 | m/z = 714.30(C₅₄H₃₈N₂ = 714.91) |
| 1-5 | m/z = 678.30(C₅₁H₃₈N₂ = 678.88) | 1-6 | m/z = 802.33(C [symbol] H [symbol] N [symbol] = 803.02) |
| 1-7 | m/z = 800.32(C₆₁H₄₀N₂ = 801.01) | 1-8 | m/z = 563.24(C₄₃H₂₉N₃ = 563.70) |
| 1-9 | m/z = 668.23(C₄₈H₃₂N₂S = 668.86) | 1-10 | m/z = 727.30(C₅₄H₃₇N₃ = 727.91) |
| 1-11 | m/z = 652.25(C₄₈H₃₂N₂O = 652.80) | 1-12 | m/z = 662.27(C₅₀H₃₄N₂ = 662.84) |
| 1-13 | m/z = 536.23(C₄₀H₂₈N₂ = 536.68) | 1-14 | m/z = 586.24(C₄₄H₃₀N₂ = 596.74) |
| 1-15 | m/z = 712.29(C₅₄H₃₆N₂ = 712.90) | 1-16 | m/z = 714.3(C₅₄H₃₈N₂ = 714.91) |
| 1-17 | m/z = 754.33(C₅₇H₄₂N₂ = 754.98) | 1-18 | m/z = 957.38(C₇₀H₄₇NS = 958.18) |
| 1-19 | m/z = 965.38(C₇₃H₄₇N₂ = 966.20) | 1-20 | m/z = 719.24(C₅ₗH₃3N₃S = 719.91) |
| 1-21 | m/z = 758.24(C₅₄H₃₄N₂OS = 758.94) | 1-22 | m/z = 893.38(C₆₇H₄₇N₃ = 894.13) |
| 1-23 | m/z = 652.25(C₄₈H₃₂N₂O = 652.80) | 1-24 | m/z = 662.27(C₅₀H₃₄N₂ = 662.84) |
| 1-25 | m/z = 562.24(C₄₂H₃₀N₂ = 562.72) | 1-26 | m/z = 612.26(C₄₆H₃₂N₂ = 612.78) |
| 1-27 | m/z = 688.29(C₅₂H₃₆N₂ = 688.87) | 1-28 | m/z = 714.30(C₅₄H₃₈N₂ = 714.91) |
| 1-29 | m/z = 754.33(C₅₇H₄₂N₂ = 754.98) | 1-30 | m/z = 878.37(C₆₇H₄₆N₂ = 879.12) |
| 1-31 | m/z = 876.35(C₆₇H₄₄N₂ = 877.10) | 1-32 | m/z = 639.27(C₄₇H₃₃N₃ = 369.80) |
| 1-33 | m/z = 768.26(C₅₆H₃₄N₂S = 768.98) | 1-34 | m/z = 833.29(C₆₀H₃₉N₃S = 834.05) |
| 1-35 | m/z = 742.26(C₅₄H₃₄N₂OS = 742.88) | 1-36 | m/z = 778.33(C₅₉H₄₂N₂ = 779.00) |
| 1-37 | m/z = 486.21(C₃₆H₂₆N₂ = 486.62) | 1-38 | m/z = 536.23(C₄₀H₂₈N₂ = 536.68) |
| 1-39 | m/z = 612.26(C₄₆H₃₂N₂ = 6 12.78) | 1-40 | m/z = 638.27(C₄₈H₃₄N₂ = 638.81) |
| 1-41 | m/z = 491.24(C₃₆H₂₁D₅N₂ = 491.65) | 1-42 | m/z = 612.26(C₄₆H₃₂N₂ = 612.78) |
| 1-43 | m/z = 794.28(C₅₈H₃₈N₂S = 795.02) | 1-44 | m/z = 656.26(C₄₈H₃₃FN₂ = 656.80) |
| 1-45 | m/z = 717.29(C₅₁H₃₅N₅ = 717.88) | 1-46 | m/z = 728.32(C₅₅H₄₀N₂ = 728.94) |
| 1-47 | m/z = 842.34(C₆₂H₄₂N₄ = 843.05) | 1-48 | m/z = 714.30(C₅₄H₃₈N₂ = 714.91) |
| 1-49 | m/z = 653.28(C₄₈H₃₅N₃ = 653.81) | 1-50 | m/z = 703.30(C₅₂H₃₇N₃ = 703.87) |
| 1-51 | m/z = 805.35(C₆₀H₄₃N₃ = 806.00) | 1-52 | m/z = 753.31(C₅₆H₃₉N₃ = 753.93) |
| 1-53 | m/z = 818.34(C₆₀H₄₂N₄ = 819.00) | 1-54 | m/z = 835.30(C₆₀H₄₁N₃S = 836.05) |
| 1-55 | m/z = 655.27(C₄₆H₃₃N₅ = 655.79) | 1-56 | m/z = 885.32(C₆₄H₄₃N₃S = 886.11) |
| 1-57 | m/z = 759.27(C₅₄H₃₇N₃S = 759.96) | 1-58 | m/z = 706.28(C₄₉H₃₄N₆ = 706.83) |
| 1-59 | m/z = 960.39(C₆₉H₄₈N₆ = 961.16) | 1-60 | m/z = 853.35(C₆₄H₄₃N₃ = 854.05) |
| 1-61 | m/z = 894.37(C₆₆H₄N₄ = 895.10) | 1-62 | m/z = 834.38(C₆₂H₃₈D₅N₃ = 835.06) |
| 1-63 | m/z = 855.36(C₆₄H₄N₃ = 856.06) | 1-64 | m/z = 853.35(C₆₄H₄₃N₃ = 854.05) |
| 1-65 | m/z = 794.37(C₆₀H₄₆N₂ = 795.04) | 1-66 | m/z = 987.39(C₇₁H₄₉N₅O = 988.21) |
| 1-67 | m/z = 102144(C₇₇H₅₅N₃ = 1022.31) | 1-68 | m/z = 737.23(C₅₁H₃₂FN₃S = 737.90) |
| 1-69 | m/z = 562.24(C₄₂H₃₀N₂ = 562.72) | 1-70 | m/z = 602.27(C₄₅H₃₄N₂ = 602.78) |
| 1-71 | m/z = 563.24(C₄₁H₂₉N₃ = 563.70) | 1-72 | m/z = 714.30(C₅₄H₃₈N₂ = 714.91) |
| 1-73 | m/z = 678.30(C₅₁H₃₈N₂ = 678.88) | 1-74 | m/z = 802.33(C₆₁H₄₂N₂ = 803.02) |
| 1-75 | m/z = 800.32(C₆₁H₄₀N₂ = 801.01) | 1-76 | m/z = 563.24(C₄₁H₂₉N₃ = 563.70) |
| 1-77 | m/z = 668.23(C₄₈H₃₂N₂S = 668.86) | 1-78 | m/z = 727.30(C₅₄H₃₇N₃ = 727.91) |
| 1-79 | m/z = 652.25(C₄₈H [symbol] N₂O = 652.80) | 1-80 | m/z = 662.27(C [symbol] H [symbol] N₂ = 662.84) |
| 1-81 | m/z = 536.23(C₄₆H₂₈N₂ = 536.68) | 1-82 | m/z = 586.24(C [symbol] H [symbol] N₂ = 586.74) |
| 1-83 | m/z = 712.29(C₅₄H₃₆N₂ = 712.90) | 1-84 | m/z = 714.30(C [symbol] H [symbol] N₂ = 714.91) |
| 1-85 | m/z = 754.33(C₅₇H₄₂N₂ = 754.98) | 1-86 | m/z = 957.38(C [symbol] H [symbol] N₅ = 958.18) |
| 1-87 | m/z = 965.38(C [symbol] H [symbol] N₃ = 966.20) | 1-88 | m/z = 719.24(C [symbol] H [symbol] N₃S = 719.91) |
| 1-89 | m/z = 758.24(C₅₄H₃₄N₂OS = 758.94) | 1-90 | m/z = 893.38(C [symbol] H [symbol] N₃ = 894.13) |
| 1-91 | m/z = 652.25(C₄₈H₃₂N₂O = 652.80) | 1-92 | m/z = 662.27(C₃₆H₃₄N₂ = 662.84) |
| 1-93 | m/z = 562.24(C₄₂H [symbol] N₂ = 562.72) | 1-94 | m/z = 612.26(C₄₆H₃₂N₂ = 612.78) |
| 1-95 | m/z = 688.29(C₅₂H₃₆N₂ = 688.87) | 1-96 | m/z = 714.30(C [symbol] H [symbol] N₂ = 714.91) |
| 1-97 | m/z = 754.33(C₅₇H₄₂N₂ = 754.98) | 1-98 | m/z = 878.37(C₆₇H₄₆N₂ = 879.12) |
| 1-99 | m/z = 876.35(C₆₇H₄₄N₂ = 877.10) | 1-100 | m/z = 639.27(C₄₇H₃₂N₃ = 369.80) |
| 1-101 | m/z = 768.26(C₅₆H₃₆N₂S = 768.98) | 1-102 | m/z = 833.29(C [symbol] H [symbol] N₃S = 834.05) |
| 1-103 | m/z = 742.26(C₅₄H₃₄N₂OS = 742.88) | 1-104 | m/z = 778.333(C [symbol] H₄₂N₂ = 779.00) |
| 1-105 | m/z = 486.21(C₃₆H₂₆N₂ = 486.62) | 1-106 | m/z = 536.23(C₄₆H₂₈N₂ = 536.68) |
| 1-107 | m/z = 612.26(C₄₆H₃₂N₂ = 612.78) | 1-108 | m/z = 638.27(C [symbol] H [symbol] N₂ = 638.81) |
| 1-109 | m/z = 491.24(C₃₆H₂₁D₅N₂ = 491.65) | 1-110 | m/z = 612.26(C₄₆H [symbol] N₂ = 612.78) |
| 1-111 | m/z = 794.28(C₅₈H₃₈N₂S = 795.02) | 1-112 | m/z = 656.26(C₄₈H₃₃FN₂ = 656.80) |
| 1-113 | m/z = 717.29(C [symbol] H₃₅N₅ = 717.88) | 1-114 | m/z = 728.32(C₃₅H₄₆N₂ = 728.94) |
| 1-115 | m/z = 842.34(C₆₂H₄₂N₄ = 843.05) | 1-116 | m/z = 714.40(C₅₄H₃₈N₂ = 714.91) |
| 1-117 | m/z = 653.28(C [symbol] H₃₅N₃ = 653.81) | 1-118 | m/z = 703.30(C₅₂H₃₂N₃ = 703.87) |
| 1-119 | m/z = 805.35(C₆₀H₄₃N₃ = 806.00) | 1-120 | m/z = 753.31(C₅₆H₃₉N₃ = 753.93) |
| 1-121 | m/z = 818.34(C₆₀H₄₂N₄ = 819.00) | 1-122 | m/z = 835.30(C₆₀H₄₁N₃S = 836.05) |
| 1-123 | m/z = 655.27(C₄₆H [symbol] N₅ = 655.79) | 1-124 | m/z = 885.32(C₆₄H [symbol] N₃S = 886.11) |
| 1-125 | m/z = 759.27(C [symbol] H [symbol] N [symbol] S = 759.96) | 1-126 | m/z = 706.28(C [symbol] H₃₄N₆ = 706.83) |
| 1-127 | m/z = 960.39(C₆₉H₄₈N₆ = 961.16)) | 1-128 | m/z = 853.35(C [symbol] H [symbol] N₃ = 854.05) |
| 1-129 | m/z = 894.37(C₆₆H₄₆N₄ = 895.10) | 1-130 | m/z = 834.38(C₆₂H₃₈D₅N₃ = 835.06) |
| 1-131 | m/z = 855.36(C₆₄H₄₅N₃ = 856.06) | 1-132 | m/z = 853.35(C₆₄H₄₃N₃ = 854.05) |
| 1-133 | m/z = 794.37(C [symbol] H₄₆N₂ = 795.04) | 1-134 | m/z = 987.39(C₇₁H₄₉N₃O = 988.21) |
| 1-135 | m/z = 1021.44(C [symbol] H [symbol] N₃ = 1022.31) | 1-136 | m/z = 737.23(C₅₁H₃₂FN₃S = 737.90) |
| 1-137 | m/z = 650.27(C₄₉H₅₄N₂ = 650.83) | | |
| 2-1 | m/z = 486.21(C₃₆H [symbol] N₂ = 486.61) | 2-2 | m/z = 541.26(C₄₆H₂₃D₅N₂ = 541.69) |
| 2-3 | m/z = 612.26(C₄₆H₃₂N₂ = 612.76) | 2-4 | m/z = 562.24(C₄₂H₃₀N₂ = 562.70) |
| 2-5 | m/z = 636.26(C₄₈H₃₂N₂ = 636.78) | 2-6 | m/z = 586.24(C₄₄H₃₀N₂ = 586.72) |
| 2-7 | m/z = 712.29(C₅₄H₃₅N₂ = 712.88) | 2-8 | m/z = 638.27(C₄₈H₃₄N₂ = 638.80) |
| 2-9 | m/z = 638.27(C₄₈H₃₄N₂ = 638.80) | 2-10 | m/z = 638.27(C₄₈H₃₄N₂ = 638.80) |
| 2-11 | m/z = 638.27(C₄₈H₃₄N₂ = 638.80) | 2-12 | m/z = 738.30(C₅₆H₃₈N₂S = 738.91) |
| 2-13 | m/z = 653.28(C₄₈H₃₅N₃ = 653.81) | 2-14 | m/z = 820.36(C [symbol] H [symbol] N₂ = 821.02) |
| 2-15 | m/z = 651.27(C₄₈H₃₃N₃ = 651.80) | 2-16 | m/z = 642.21(C₄₆H₃₀N₂S = 642.21) |
| 2-17 | m/z = 663.23(C₄₈H₃₂N₂S = 668.85) | 2-18 | m/z = 668.23(C₄₈H₃₂N₂S = 668.85) |
| 2-19 | m/z = 692.23(C₅₀H₃₂N₂S = 692.87) | 2-20 | m/z = 708.26(C₅₁H₃₆N₂S = 708.91) |
| 2-21 | m/z = 794.28(C₅₈H₃₈N₂S = 795.00) | 2-22 | m/z = 698.19(C₄₈H₃₀N₂S₂ = 698.90) |
| 2-23 | m/z = 652.25(C₄₈H₃₂N₂O = 652.73) | 2-24 | m/z = 778.30(C₅₈H₃₈N₂O = 778.94) |
| 2-25 | m/z = 753.28(C₅₃H₃₅N₃O = 753.89) | 2-26 | m/z = 666.23(C₄₈H₃₀N₂O₂ = 666.76) |
| 2-27 | m/z = 682.21(C₄₀H₃₀N₂OS = 682.83) | 2-28 | m/z = 682.21(C₄₈H₃₀N₂OS = 682.83) |
| 2-29 | m/z = 678.30(C₅₁H₃₈N₂ = 678.86) | 2-30 | m/z = 702.30(C₅₃H₃₈N₂ = 702.88) |
| 2-31 | m/z = 692.28(C₅₁H₃₅N₂O = 692.84) | 2-32 | m/z = 708.26(C₅₁H₃₉N₂S = 708.91) |
| 2-33 | m/z = 794.37(C₆₀H₄₆N₂ = 795.02) | 2-34 | m/z = 802.33(C₆₁H₄₂N₂ = 803.00) |
| 2-35 | m/z = 879.36(C₆₆H [symbol] N₂ = 880.08) | 2-36 | m/z = 842.37(C₆₄H₄₆N₂ = 843.06) |
| 2-37 | m/z = 832.29(C₆₁H₄₀N₂ = 635.23) | 2-38 | m/z = 724.29(C₅₅H₃₆N₂ = 724.89) |
| 2-39 | m/z = 800.32(C₆₁H₄₀N₂ = 800.98) | 2-40 | m/z = 840.35(C₆₄H₄₄N₂ = 841.05) |
| 2-41 | m/z = 830.28(C₆₁H₃₈N₂S = 831.03) | 2-42 | m/z = 814.30(C₆₁H₃₈N₂O = 814.97) |
| 2-43 | m/z = 638.27(C₄₂H₃₄N₂ = 638.80) | 2-44 | m/z = 803.33(C [symbol] H₄₁N₂ = 803.99) |
| 2-45 | m/z = 638.27(C₄₈H₃₄N₂ = 638.80) | 2-46 | m/z = 668.23(C₄₈H₃₂N₂S = 668.85) |
| 2-47 | m/z = 578.30(C₅₁H₃₈N₂ = 678.85) | 2-48 | m/z = 835.30(C₆₀H₄₁N₃S = 836.05) |
| 2-49 | m/z = 682.21(C₄₈H₃₀NOS = 682.83) | 2-50 | m/z = 668.23(C₄₈H₃₂N₂S = 668.85) |
| 2-51 | m/z = 612.26(C₄₆H₃₂N₂ = 612.76) | 2-52 | m/z = 638.27(C₄₆H₃₄N₂ = 638.80) |
| 2-53 | m/z = 782.24(C₅₆H₃₄N₂OS = 782.95) | 2-54 | m/z = 790.33(C₆₀H₄₂N₂ = 790.99) |
| 2-55 | m/z = 805.31(C₅₉H₃₉N₃O = 805.96) | 2-56 | m/z = 664.29(C₅₀H₃₆N₂ = 664.83) |
| 2-57 | m/z = 803.33(C₆₀H₄₁N₃ = 203.99) | 2-58 | m/z = 768.26(C₅₆H₃₆N₂S = 768.96) |
| 2-59 | m/z = 650.27(C₄₉H₃₄N₂ = 650.81) | 2-60 | m/z = 688.29(C₅₂H₃₆N2 = 688.86) |
| 2-61 | m/z = 744.26(C₅₄H₃₅N₂S = 744.94) | 2-62 | m/z = 667.21(C₄₇H₂₉N₃S = 667.82) |
| 2-63 | m/z = 642.21(C₄₆H₃₀N₂S = 642.81) | 2-64 | m/z = 566.18(C₄₀H₂₆N₂S = 566.71) |
| 2-65 | m/z = 699.18(C₄₇H₂₉N₃S₂ = 699.88) | 2-66 | m/z = 682.21(C₄₈H₃₀N₂OS = 682.83) |
| 2-67 | m/z = 742.24(C₅₄H₃₄N₂S = 742.93) | 2-68 | m/z = 652.25(C₄₈H₃₂N₂O = 652.78) |
| 2-69 | m/z = 652.25(C₄₈H₃₂N₂O = 652.78) | 2-70 | m/z = 678.30(C₅₁H₃₈N₂ = 678.86) |
| 2-71 | m/z = 657.32(C₄₉H₃₁D₅N₂ = 657.85) | 2-72 | m/z = 576.26(C₄₃H₃₂N₂ = 576.73) |
| 2-73 | m/z = 642.30(C₄₈H₃₈N₂ = 642.83) | 2-74 | m/z = 766.33(C₅₈H₄₂N₂ = 766.97) |
| 2-75 | m/z = 767.33(C₅₇H₄₁N₃ = 767.96) | 2-76 | m/z = 708.26(C₅₁H₃₆N₂S = 708.91) |
| 2-77 | m/z = 692.28(C₅₁H₃₅N₂O = 692.84) | 2-78 | m/z = 706.26(C₅₁H₃₄N₂O₂ = 706.83) |
| 2-79 | m/z = 722.24(C₅₁H₃₄N₂OS = 722.89) | 2-80 | m/z = 666.27(C₄₉H₃₄N₂O = 666.81) |
| 2-81 | m/z = 603.27(C₄₄H₃₃N₃ = 603.78) | 2-82 | m/z = 778.33(C₅₉H₄₂N₂ = 778.98) |
| 2-83 | m/z = 755.33(C₅₆H₄₁N₃ = 755.94) | 2-84 | m/z = 802.33(C₆₁H₄₂N₂ = 803.00) |
| 2-85 | m/z = 778.31(C₅₇H₃₃N₄ = 778.94) | 2-86 | m/z = 890.37(C [symbol] H₄₆N₂ = 891.11) |
| 2-87 | m/z = 758.26(C₅₅H [symbol] N₂S = 756.95) | 2-88 | m/z = 846.27(C₆₁H₄₇N₂OS = 847.03) |
| 2-89 | m/z = 776.32(C₅₉H [symbol] N₂ = 776.96) | 2-90 | m/z = 548.26(C₄₉H₃₂N₂ = 648.79) |
| 2-91 | m/z = 800.32(C₆₁H₄₀N₂ = 800.98) | 2-92 | m/z = 830.28(C₆₁H₃₈N₂S = 831.03) |
| 2-93 | m/z = 864.35(C₆₅H₄₀N₂O = 865.03) | 2-94 | m/z = 840.35(C₆₄H₄₄N₂ = 841.05) |
| 2-95 | m/z = 938.35(C₇₂H₄₄N₂ = 937.13) | 2-96 | m/z = 844.25(C₆₁H₃₆N₂OS = 845.02) |
| 2-97 | m/z = 927.36(C₇₀H₄₅N₂ = 928.13) | 2-98 | m/z = 688.29(C₅₂H₃₆N₂ = 688.86) |
| 2-99 | m/z = 652.29(C₄₉H₃₃N₂ = 652.82) | 2-100 | m/z = 826.33(C₆₃H₄₂N₂ = 827.02) |
| 2-101 | m/z = 702.27(C₅₂H₃₄N₂O = 702.84) | 2-102 | m/z = 888.29(C₅₂H₃₆N₂ = 688.86) |
| 2-103 | m/z = 728.32(C₅₅H₄₀N₂ = 728.92) | 2-104 | m/z = 884.29(C₅₄H₄₀N₂OS = 885.08) |
| 2-105 | m/z = 586.24(C₄₄H₃₀N₂ = 586.72) | 2-106 | m/z = 718.24(C₅₂H₃₄N₂S = 718.90) |
| 2-107 | m/z = 732.22(C₅₂H₃₂N₂OS = 732.89) | 2-108 | m/z = 702.30(C₅₃H₃₈N₂ = 702.88) |
| 2-109 | m/z = 688.29(C₅₂H₃₆N₂ = 688.86) | 2-110 | m/z = 702.27(C₅₂H₃₄N₂O = 702.84) |
| 2-111 | m/z = 692.23(C₅₀H₃₂N₂S = 692.87) | 2-112 | m/z = 782.24(C₅₆H₃₄N₂OS = 782.95) |
| 2-113 | m/z = 738.30(C₅₆H₃₈N₂ = 738.91) | 2-114 | m/z = 768.26(C₅₆H₃₆N₂S = 768.96) |
| 2-115 | m/z =715.32(C₅₄H₄₀N₂ = 716.91) | 2-116 | m/z = 857.29(C₆₂H₃₉N₃S = 858.06) |
| 2-117 | m/z = 738.30(C₅₆H₃₃N₂ = 738.91) | 2-118 | m/z = 753.28(C₅₅H₃₅N₃O = 753.89) |
| 2-119 | m/z = 677.28(C₅₀H₃₃N₂ = 677.83) | 2-120 | m/z = 679.32(C₆₅H₄₁N₃O = 880.04) |
| 2-121 | m/z = 812.26(C₄₆H₃₂N₂ = 612.76) | 2-122 | m/z = 756.31(C₅₆H₄₀N₂N = 756.93) |
| 2-123 | m/z = 727.30(C₅₄H₃₇N₂ = 727.89) | 2-124 | m/z = 866.37(C₆₆H₄₆N₂ = 867.08) |
| 3-1 | m/z = 503.17(C₃₆H₂₅NS = 503.66) | 3-2 | m/z = 603.20(C₄₄H₂₉NS = 603.77) |
| 3-3 | m/z = 477.16(C₃₄H₂₃NS = 477.62) | 3-4 | m/z = 503.17(C₃₆H₂₅NS = 503.66) |
| 3-5 | m/z = 451.14(C₃₂H₂₁NS = 451.58) | 3-6 | m/z = 593.22(C₄₃H₃₁NS = 593.78) |
| 3-7 | m/z = 641.22(C₄₇H₃₁NS = 641.82) | 3-8 | m/z = 665.22(C₄₉H₃₁NS = 665.84) |
| 3-9 | m/z = 503.17(C₃₀H₂₅NS = 503.66) | 3-10 | m/z = 655.23(C₄₈H₃₃NS = 655.85) |
| 3-11 | m/z = 695.26(C₅₁H₃₇NS = 695.91) | 3-12 | m/z = 593.18(C₄₂H₂₇NOS = 593.73) |
| 3-13 | m/z = 583.14(C₄₀H₂₃NS = 583.76) | 3-14 | m/z = 579.20(C₄₂H₂₉NS = 579.75) |
| 3-15 | m/z = 685.19(C₄₈H₃₁NS₂ = 685.90) | 3-16 | m/z = 719.23(C₅₂H₃₃NOS = 719.89) |
| 3-17 | m/z = 629.22(C₄₆H₃₁NS = 629.81) | 3-18 | m/z = 629.22(C₄₆H₃₁NS = 629.81) |
| 3-19 | m/z = 609.20(C₄₄H₂₉NS = 803.77) | 3-20 | m/z = 553.08(C₃₆H₂₁NS = 563.75) |
| 3-21 | m/z = 639.11(C₄₂H₂₅NS₃ = 639.85) | 3-22 | m/z = 715.15(C₄₈H₂₉NS₃ = 715.95) |
| 3-23 | m/z = 791.18(C₅₄H₃₃NS₃ = 792.04) | 3-24 | m/z = 607.16(C₄₂H₂₅NO₂S = 607.72) |
| 3-25 | m/z = 633.21(C₄₅H₃₁NOS = 633.80) | 3-26 | m/z = 733.24(C₅₃H₃₅NOS = 733.92) |
| 3-27 | m/z = 883.29(C₆₅H₄₁NOS = 884.09) | 3-28 | m/z = 535.13(C [symbol] H₂₃N₄S₂ = 525.74) |
| 3-29 | m/z = 553.19(C₄₀H₂₇NS = 553.71) | 3-30 | m/z = 603.20(C₄₄H₂₉NS = 603.77) |
| 3-31 | m/z = 841.28(C₆₃H₃₉NS = 842.06) | 3-32 | m/z = 567.17(C₄₀H₂₅NOS = 567.71) |
| 3-33 | m/z = 563.22(C₄₂H₂₉NO = 563.69) | 3-34 | m/z = 563.22(C₄₂H₂₉NO = 563.69) |
| 3-35 | m/z = 613.24(C₄₆H₃₁NO = 613.74) | 3-36 | m/z = 703.29(C₅₃H₃₇NO = 703.87) |
| 3-37 | m/z = 587.22(C₄₄H₂₉NO = 587.71) | 3-38 | m/z = 639.26(C₄₅H₃₃NO = 639.78) |
| 3-39 | m/z = 639.26(C₄₈H₃₃NO = 639.78) | 3-40 | m/z = 653.24(C₄₈H₃₁NO₂ = 653.77) |
| 3-41 | m/z = 603.26(C₄₅H₃₃NO = 603.75) | 3-42 | m/z = 727.29(C₅₅H₃₇NO = 727.89) |
| 3-43 | m/z = 725.27(C₅₅H₄₆NO = 725.87) | 3-44 | m/z = 595.17(C₄₆H₂₅N₃OS = 595.71) |
| 3-45 | m/z = 567.26(C₄₂H₃₃NO = 567.72) | 3-46 | m/z = 611.22(C₄₆H₂₉NO = 611.73) |
| 3-47 | m/z = 617.18(C₄₄H₂₇NOS = 617.76) | 3-48 | m/z = 637.24(C [symbol] H₃₁NO = 637.77) |
| 3-49 | m/z = 667.21(C₄₈H₂₉NO₃ = 667.75) | 3-50 | m/z = 767.25(C₅₆H₃₃NO₂ = 767.87) |
| 3-51 | m/z = 681.27(C₅₀H₃₅NO₂ = 681.82) | 3-52 | m/z = 567.17(C₄₀H₂₅NOS = 567.71 |
| 3-53 | m/z = 658.22(C₄₅H₃₀N₄S = 658.82) | 3-54 | m/z = 655.23(C₄₈H₃₃NS = 655.86) |
| 3-55 | m/z = 744.26(C₅₄H₃₆N₂S = 744.96) | 3-56 | m/z = 784.27(C₅₅H₃₆N₄S = 784.98) |
| 3-57 | m/z = 553.19(C₄₀H₂₇NS = 553.72) | 3-58 | m/z = 553.19(C₄₀H₂₇NS = 553.72) |
| 3-59 | m/z = 543.2(C₃₃H₂₉NS = 543.73) | 3-60 | m/z = 671.21(C₄₈H₃₀FNS = 671.83) |
| 3-61 | m/z = 641.25(C₄₆H₃₁N₂O = 641.77) | 3-62 | m/z = 639.26(C₄₈H₃₃NO = 639.8) |
| 3-63 | m/z = 652.25(C₄₈H₃₂N₂O = 652.8) | 3-64 | m/z = 667.25(C₄₈H₃₃NO₂ = 667.81) |
| 3-65 | m/z = 557.17(C₄₀H₂₅NOS = 567.71) | 3-66 | m/z = 557.17(C₄₀H₂₅NOS = 567.71) |
| 3-67 | m/z = 731.23(C₅₃H₃₃NOS = 731.91) | 3-68 | m/z = 731.23(C₅₃H₃₃NOS = 731.91) |
| 3-69 | m/z = 683.23(C₄₉H₃₃NOS = 683.87) | 3-70 | m/z = 551.19(C₄₀H₂₅NO₂ = 551.65) |
| 3-71 | m/z = 643.2(C₄₆H₂₉NOS = 643.8) | 3-72 | m/z = 601.2(C₄₄H₂₇NO₂ = 601.71) |
| 3-73 | m/z = 607.2(C₄₃H₂₃NOS = 607.77) | 3-74 | m/z = 701.28(C₅₀H₃₉NOS = 701.93) |
| 3-75 | m/z = 577.24(C₄₃H₃₁NO = 577.73) | 3-76 | m/z = 567.17(C₄₆H₂₅NOS = 567.71) |
| 4-1 | m/z = 513.25(C₃₉H₃₁N = 513.68) | 4-2 | m/z = 613.28(C₄₇H₃₅N = 613.20) |
| 4-3 | m/z = 665.31(C₅₁H₃₉N = 665.88) | 4-4 | m/z = 705.34(C₅₄H₄₃N = 705.55) |
| 4-5 | m/z = 593.31(C₄₅H₃₉N = 593.81) | 4-6 | m/z = 589.28(C₄₅H₃₅N = 589.78) |
| 4-7 | m/z = 513.25(C₃₉H₃₁N = 513.68) | 4-8 | m/z = 639.29(C₄₉H₃₇N = 639.84) |
| 4-9 | m/z = 589.28(C₄₅H₃₅N = 589.78) | 4-10 | m/z = 665.31(C₅₁H₃₉N = 665.88) |
| 4-11 | m/z = 553.28(C₄₂H₃₅N = 553.75) | 4-12 | m/z = 669.34(C₅₁H₄₃N = 669.91) |
| 4-13 | m/z = 779.36(C₆₀H₄₅N = 780.03) | 4-14 | m/z = 761.35(C₅₆H₄₇NSi = 762.08) |
| 4-15 | m/z = 705.34(C₅₄H₄₃N = 705.95) | 4-16 | m/z = 589.28(C₄₅H₃₅N = 589.78) |
| 4-17 | m/z = 665.31(C₅₁H₃₉N = 665.88) | 4-18 | m/z = 728.32(C₅₅H₄₀N₂ = 728.94) |
| 4-19 | m/z = 637.28(C₄₉H₃₅N = 637.83) | 4-20 | m/z = 789.34(C₆₁H₄₃N = 790.02) |
| 4-21 | m/z = 677.31(C₅₂H₃₉N = 677.89) | 4-22 | m/z = 775.32(C₆₀H₄₁N = 776.00) |
| 4-23 | m/z = 801.34(C₆₂H₄₃N = 802.03) | 4-24 | m/z = 799.32(C₆₂H₄₁N = 800.02) |
| 4-25 | m/z = 965.40(C₅₆H₄₁N = 966.24) | 4-26 | m/z = 637.28(C₄₉H₃₅N = 637.83) |
| 4-27 | m/z = 635.26(C₄₉H₃₃N = 635.81) | 4-28 | m/z = 616.29(C₄₇H₂₈D₅N = 616.82) |
| 4-29 | m/z = 727.32(C₅₆H₄₁N = 727.95) | 4-30 | m/z = 687.29(C₅₃H₃₇N = 687.89) |
| 4-31 | m/z = 877.37(C₆₈H₄₇N = 878.13) | 4-32 | m/z = 753.34(C₅₈H₄₃N = 753.99) |
| 4-33 | m/z = 689.31(C₅₃H₃₉N = 689.90) | 4-34 | m/z = 637.28(C₄₉H₃₅N = 637.83) |
| 4-35 | m/z = 850.32(C₆₂H₄₀F₂N₂ = 851.01) | 4-36 | m/z = 559.23(C₄₃H₂₉N = 559.71) |
| 4-37 | m/z = 635.26(C₄₉H₃₈N = 635.81) | 4-38 | m/z = 663.29(C₅₁H₃₇N = 663.86) |
| 4-39 | m/z = 735.29(C₅₇H₃₇N = 735.93) | 4-40 | m/z = 735.29(C₅₇H₃₇N = 735.93) |
| 4-41 | m/z = 725.31(C₅₆H₃₉N = 725.94) | 4-42 | m/z = 735.29(C₅₃H₃₇N = 735.93) |
| 4-43 | m/z = 751.32(C₅₈H₄₁N = 751.97) | 4-44 | m/z = 725.31(C₅₆H₃₉N = 725.94) |
| 4-45 | m/z = 675.29(C₅₆H₃₉N = 675.88) | 4-46 | m/z = 675.29(C₅₂H₃₇N = 675.88) |
| 4-47 | m/z = 751.32(C₅₈H₄₁N = 751.97) | 4-48 | m/z = 675.29(C₅₂H₃₇N = 675.88) |
| 4-49 | m/z = 721.28(C₅₆H₃₅N = 721.90) | 4-50 | m/z = 797.31(C₆₂H₃₉N = 798.00) |
| 4-51 | m/z = 959.36(C₇₅H₄₅N = 960.19) | 4-52 | m/z = 607.23(C₄₇H₂₉N = 607.76) |
| 4-53 | m/z = 675.29(C₅₂H₃₇N = 675.88) | 4-54 | m/z = 635.26(C₄₉H₃₃N = 635.81) |
| 4-55 | m/z = 640.29(C₄₉H₂₈D₅N = 640.84) | 4-56 | m/z = 685.28(C₅₃H₃₅N = 685.87) |
| 4-57 | m/z = 735.29(C₅₇H₃₇N = 735.93) | 4-58 | m/z = 583.23(C₄₅H₂₉N = 583.73) |
| 4-59 | m/z = 725.31(C₅₆H₃₉N = 725.94) | 4-60 | m/z = 725.31(C₅₆H₃₉N = 725.94) |
| 4-61 | m/z = 635.26(C₄₉H₃₈N = 635.81) | 4-62 | m/z = 751.32(C₅₈H₄₁N = 751.97) |
| 4-63 | m/z = 751.32(C₅₈H₄₁N = 751.97) | 4-64 | m/z = 751.32(C₅₈H₄₁N = 751.97) |
| 4-65 | m/z = 842.37(C₆₄H₄₈N₂ = 843.09) | 4-66 | m/z = 751.32(C₅₈H₄₁N = 751.97) |
| 4-67 | m/z = 873.34(C₆₈H₄₃N = 874.10) | 4-68 | m/z = 711.29(C₅₅H₃₇N = 711.91) |
| 4-69 | m/z = 751.32(C₅₈H₄₁N = 751.97) | 4-70 | m/z = 787.32(C₆₁H₄₁N = 609.77) |
| 5-1 | m/z = 583.23(C₄₅H₂₃N = 583.73) | 5-2 | m/z = 609.25(C₄₇H₃₁N = 609.77) |
| 5-3 | m/z = 685.28(C₅₃H₃₅N = 685.87) | 5-4 | m/z = 659.26(C₅₁H₄₃N = 659.83) |
| 5-5 | m/z = 607.29(C₄₇H₂₉N = 607.76) | 5-6 | m/z = 685.28(C₅₃H₃₅N = 685.87) |
| 5-7 | m/z = 664.29(C₅₃H₂₈D₅N = 664.86) | 5-8 | m/z = 648.28(C₅₀H₃₅N = 644.84) |
| 5-9 | m/z = 699.29(C₅₄H₃₇N = 699.500 | 5-10 | m/z = 699.29(C₅₄H₃₇N = 699.90) |
| 5-11 | m/z = 726.31(C₅₆H₃₉N = 725.94) | 5-12 | m/z = 739.32(C₅₃H₄₁N = 739.96) |
| 5-13 | m/z = 730.34(C₅₆H₃₄D₅N = 739.97) | 5-14 | m/z = 775.32(C₆₀H₄₁N = 776.00) |
| 5-15 | m/z = 775.32(C [symbol] H [symbol] N = 776.00) | 5-16 | m/z = 775.32(C₆₀H₄₁N = 776.00) |
| 5-17 | m/z = 725.31(C₅₆H₃₉N = 725.94) | 5-18 | m/z = 753.32(C₆₀H₃₉N = 773.98) |
| 5-19 | m/z = 725.31(C₅₆H₃₉N = 725.94) | 5-20 | m/z = 774.30(C [symbol] H₃₈N₂ = 774.97) |
| 5-21 | m/z = 639.20(C₄₇H₂₉NS = 639.82) | 5-22 | m/z = 699.26(C₅₃H₃₃NO = 699.85) |
| 5-23 | m/z = 775.29(C₅₉H₃₇NO = 775.95) | 5-24 | m/z = 775.29(C₅₉H₃₇NO = 775.95) |
| 5-25 | m/z = 855.30(C₆₅H₃₉NO = 866.03) | 5-26 | m/z = 583.23(C₄₅H₂₉N = 583.73) |
| 5-27 | m/z = 685.28(C₅₃H₃₅N = 685.87) | 5-28 | m/z = 649.28(C [symbol] H₃₅N = 649.84) |
| 5-29 | m/z = 699.29(C₅₄H₃₇N = 699.90) | 5-30 | m/z = 725.31(C₅₆H₃₉N = 725.94) |
| 5-31 | m/z = 725.31(C₅₆H₃₃N = 725.94) | 5-32 | m/z = 775.32(C₆₀H₄₁N = 776.00) |
| 5-33 | m/z = 775.32(C₆₀H₄₁N = 776.00) | 5-34 | m/z = 775.32(C₆₀H₄₁N = 776.00) |
| 5-35 | m/z = 699.29(C₅₄H₃₇N = 699.90) | 5-36 | m/z = 773.31(C₆₀H₃₉N = 773.98) |
| 5-37 | m/z = 715.23(C₅₃H₃₃NS = 715.91) | 5-38 | m/z = 739.29(C₅₆H₃₇NO = 739.92) |
| 5-39 | m/z = 699.26(C₅₃H₃₃NO = 599.85) | 5-40 | m/z = 652.25(C₄₈H₃₀N₄ = 662.50) |
| 5-41 | m/z = 685.28(C₅₃H₃₅N = 685.87) | 5-42 | m/z = 685.28(C₅₃H₃₅N = 685.87) |
| 5-43 | m/z = 633.25(C₄₉H₃₁N = 633.79) | 5-44 | m/z = 649.28(C [symbol] H₃₅N = 649.84) |
| 5-45 | m/z = 725.31(C₅₆H₃₉N = 725.94) | 5-46 | m/z = 725.31(C₅₆H₃₉N = 725.94) |
| 5-47 | m/z = 801.34(C [symbol] H [symbol] N = 802.03) | 5-48 | m/z = 725.31(C₅₈H₃₉N = 725.94) |
| 5-49 | m/z = 849.34(C₆₆H₄₃N = 850.08) | 5-50 | m/z = 850.33(C₆₅H₄₂N₂ = 851.07) |
| 5-51 | m/z = 689.22(C₅₃H₃₃NS = 889.88) | 5-52 | m/z = 623.22(C₄₇H₂₉NO = 623.76) |
| 5-53 | m/z = 775.29(C₅₉H₃₇NO = 778.95) | 5-54 | m/z = 699.26(C₅₃H₃₃NO = 699.85) |
| 5-55 | m/z = 715.23(C₅₃H₃₃NS = 715.91) | 5-56 | m/z = 690.31(C₅₃H [symbol] D₅N = 690.90) |
| 5-57 | m/z = 787.32(C₆₁H₄₁N = 788.01) | 5-58 | m/z = 699.29(C₅₄H₃₇N = 699.80) |
| 5-59 | m/z = 725.31(C₅₆H₃₉N = 725.54) | 5-60 | m/z = 715.23(C₅₃H₃₃NS = 715.91) |
| 5-61 | m/z = 633.25(C₄₉H₃₁N = 633.79) | 5-62 | m/z = 583.23(C₄₅H₂₉N = 583.73) |
| 5-63 | m/z = 865.33(C [symbol] H₄₃NO = 866.06) | 5-64 | m/z = 821.31(C₆₄H₃₉N = 822.03 |
| 5-65 | m/z = 690.27(C₅₃H₃₄N₂ = 698.87) | 5-66 | m/z = 659.26(C₅₃H₃₃N = 659.83) |
| 5-67 | m/z = 619.28(C₅₆H₃₅N = 649.84) | 5-68 | m/z = 99.344(C₆₆H₄₃N = 950.08) |
| 5-69 | m/z = 850.33(C₆₅H₄₂N₂ = 851.07) | 5-70 | m/z = 825.30(C₆₃H₃₉NO = 826.01) |
| 5-71 | m/z = 583.23(C₄₅H₂₃N = 533.77) | 5-72 | m/z = 685.28(C₅₃H₃₅N = 505.87) |
| 5-73 | m/z = 693.32(C₅₃H₂₃D [symbol] N = 693.92) | 5-74 | m/z = 699.29(C₅₃H₃₇N = 639.90) |
| 5-75 | m/z = 725.31(C₅₆H₃₉N = 725.94) | 5-76 | m/z = 775.32(C [symbol] H₄₁N = 776.60) |
| 5-77 | m/z = 725.31(C₅₆H₃₉N = 725.54) | 5-78 | m/z = 849.34(C₆₆H₄₃N = 850.08) |
| 5-79 | m/z = 755.31(C₅₇H₃₉N₃ = 765.96) | 5-80 | m/z = 775.29(C₅₉H₃₇NO = 775.95) |
| 5-81 | m/z = 685.28(C₅₃H₃₅N = 685.87) | 5-82 | m/z = 609.25(C₄₇H₃₁N = 609.77) |
| 5-83 | m/z = 785.31(C₆₁H₃₉N = 785.99) | 5-84 | m/z = 725.31(C₅₆H₃₉N = 725.94) |
| 5-85 | m/z = 729.21(C₅₃H₃₁NOS = 728.90) | 5-86 | m/z = 583.23(C₄₆H₂₉N = 583.73) |
| 5-87 | m/z = 685.28(C₅₃H₃₅N = 688.87) | 5-88 | m/z = 735.29(C₅₇H₃₇N=) |
| 5-89 | m/z = 685.28(C₅₃H₃₅N = 685.87) | 5-90 | m/z = 685.28(C₅₃H₃₅N = 685.87) |
| 5-91 | m/z = 725.31(C₅₆H₃₉N = 725.94) | 5-92 | m/z = 749.31(C₅₈H₃₉N = 749.96) |
| 5-93 | m/z = 725.31(C₅₆H₃₉N = 725.94) | 5-94 | m/z = 725.31(C₅₆H₃₉N = 725.94) |
| 5-95 | m/z = 725.31(C₅₆H₃₉N = 725.94) | 5-96 | m/z = 773.31(C₆₀H₃₉N = 773.98) |
| 5-97 | m/z = 850.33(C₆₅H₄₂N₂ = 851.07) | 5-98 | m/z = 699.26(C₅₃H₃₃N = 699.85) |
| 5-99 | m/z = 715.23(C₅₃H₃₃NS = 715.91) | 5-100 | m/z = 739.29(C₅₆H₃₇NO = 739.92) |
| 5-101 | m/z = 709.28(C₅₅H₃₅N = 709.89) | 5-102 | m/z = 749.31(C₅₈H₃₉N = 749.96) |
| 5-103 | m/z = 901.37(C₇₀H₄₇N = 902.15) | 5-104 | m/z = 660.26(C₅₀H₃₂N₂ = 660.82) |
| 5-105 | m/z = 673.24(C₅₁H₃₁NO = 673.82) | 5-106 | m/z = 685.28(C₅₃H₃₅N = 685.87) |
| 5-107 | m/z = 775.32(C₆₀H₄₁N = 776.00) | 5-108 | m/z = 772.29(C59H36N2 = 772.95) |
| 5-109 | m/z = 928.38(C₇₁H₄₈N₂ = 929.18) | 5-110 | m/z = 941.31(C₇₁H₄₃NS = 942.19) |
| 5-111 | m/z = 836.32(C₅₁H₄₀N₂ = 837.04) | 5-112 | m/z = 913.35(C₆₉H₄₃N₃ = 914.12) |
| 5-113 | m/z = 800.28(C₆₀H₃₆N₂O = 800.96) | 5-114 | m/z = 841.28(C₆₃H₃₉NS = 842.07) |
| 5-115 | m/z = 877.37(C₆₈H₄₇N = 878.13) | | |
| 6-1 | m/z = 713.31(C₅₅H₃₉N = 713.92) | 6-2 | m/z = 585.28(C₄₃H₃₅N = 589.78) |
| 6-3 | m/z = 639.29(C₄₉H₃₇N = 639.84) | 6-4 | m/z = 613.28(C₄₇H₃₅N = 613.8) |
| 6-5 | m/z = 601.28(C₄₆H₃₅N = 601.79) | 6-6 | m/z = 677.31(C₅₂H₃₉N = 677.89) |
| 6-7 | m/z = 777.3(C [symbol] H₃₉NO = 777.97) | 6-8 | m/z = 651.26(C₄₉H₃₃NO = 651.81) |
| 6-9 | m/z = 577.24(C₄₃H₃₇NO = 577.73) | 6-10 | m/z = 593.22(C₄₃H₃₅NS = 593.79) |
| 6-11 | m/z = 577.24(C₄₃H₃₇NO = 577.73) | 6-12 | m/z = 527.26(C₄₇H₃₃NO = 827.79) |
| 6-13 | m/z = 679.32(C₅₂H₄₁N = 679.91) | 6-14 | m/z = 651.29(C₅₀H₃₇N = 651.85) |
| 6-15 | m/z = 725.31(C₅₆H₃₉N = 725.94) | 6-16 | m/z = 725.31(C₅₆H₃₉N = 725.94) |
| 6-17 | m/z = 677.31(C₅₂H₃₉N = 677.89) | 6-18 | m/z = 801.28(C₄₆H₃₅N = 601.79) |
| 6-19 | m/z = 691.29(C₅₂H₃₇NO = 691.87) | 6-20 | m/z = 691.29(C₅₂H₃₇NO = 691.87) |
| 6-21 | m/z = 691.29(C₅₂H₃₇NO = 691.87) | 6-22 | m/z = 689.27(C₅₂H₃₅N = 689.26) |
| 6-23 | m/z = 767.32(C₅₈H₄₁NO =767.97) | 6-24 | m/z = 767.32(C₅₈H₄₁NO = 767.97) |
| 6-25 | m/z = 767.32(C₅₈H₄₁NO = 767.97) | 6-26 | m/z = 765.30(C₅₈H₃₉NO = 765.96) |
| 6-27 | m/z = 677.31(C₅₂H₃₉N = 677.89) | 6-28 | m/z = 753.34(C₅₈H₄₃N = 753.99) |
| 6-29 | m/z = 753.34(C₅₈H₄₃N = 753.99) | 6-30 | m/z = 819.33(C₆₁H₄₅NS = 820.12) |
| 6-31 | m/z = 824.36(C₆₁H [symbol] D₅NSi = 825.15) | | |
| 11-1 | m/z = 473.21(C₃₆H₂₇N = 473.61) | 11-2 | m/z = 523.23(C₄₀H₂₉N = 523.66) |
| 11-3 | m/z = 573.25(C₄₄H₃₁N = 573.72) | 11-4 | m/z = 623.26(C₄₈H₃₃N = 623.78) |
| 11-5 | m/z = 447.20(C₃₄H₂₅N = 447.57) | 11-6 | m/z = 371.17(C₂₈H₂₅N = 371.47) |
| 11-7 | m/z = 471.20(C₃₆H₂₅N = 471.59) | 11-8 | m/z = 521.21(C₄₀H₂₇N = 521.65) |
| 11-9 | m/z = 549.25(C₄₂H₃₁N = 549.70) | 11-10 | m/z = 625.28(C₄₈H₃₅N = 625.80) |
| 11-11 | m/z = 675.29(C₅₂H₃₇N = 675.86) | 11-12 | m/z = 473.21(C₃₆H₂₇N = 473.61) |
| 11-13 | m/z = 523.23(C₄₀H₃₉N = 523.66) | 11-14 | m/z = 623.26(C₄₈H₃₃N = 623.78) |
| 11-15 | m/z = 549.25(C₄₂H₃₃N = 549.70) | 11-16 | m/z = 625.28(C₄₈H₃₅N = 625.80) |
| 11-17 | m/z = 473.21(C₃₆H₃₇N = 473,62) | 11-18 | m/z = 725.31(C₅₆H₃₉N = 725.94) |
| 11-19 | m/z = 625.28(C₄₈H₃₅N = 625.82) | 11-20 | m/z = 749.31(C₅₈H₃₉N = 749.96) |
| 11-21 | m/z = 699.29(C₅₄H₃₇N = 699.9) | 11-22 | m/z = 730.34(C₅₆H₃₅D₅N = 730.97) |
| 11-23 | m/z = 753.33(C₅₈H₃₅D₄N = 753.98) | | |

[symbol]

indicates data missing or illegible when filed

### Synthesis Example 2

A compound (e.g., the fourth compound, the sixth compound, the seventh compound, or the eighth compound described above) represented by Formula D and including a radical of a compound Formula A or Formula B, according to the present disclosure, is prepared by, but is not limited to, reacting Sub 3-A to Sub 3-D with Sub 4 as in the following Reaction Formula 9.

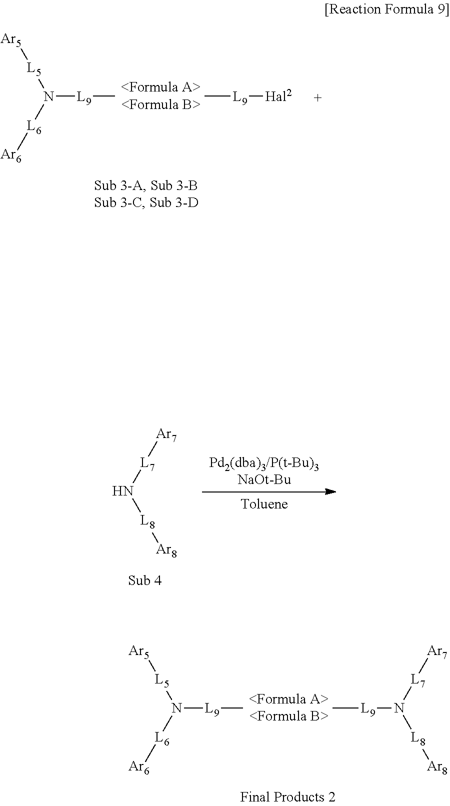

More specifically, Reaction Formula 8 may be represented by, but is not limited to, the following Reaction Formula 10 to Reaction Formula 13.

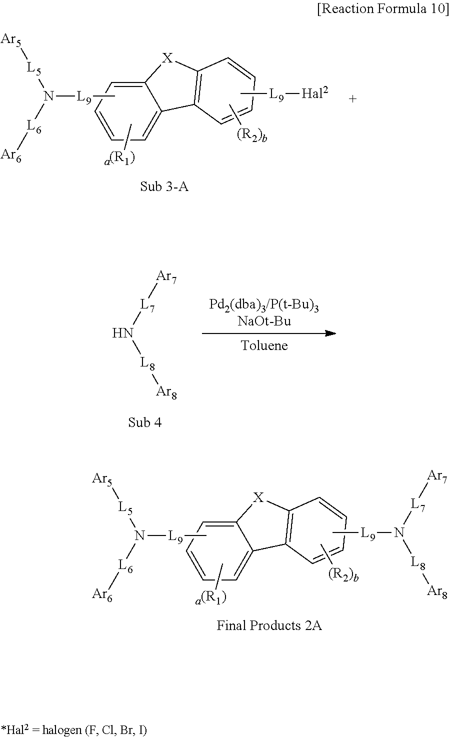

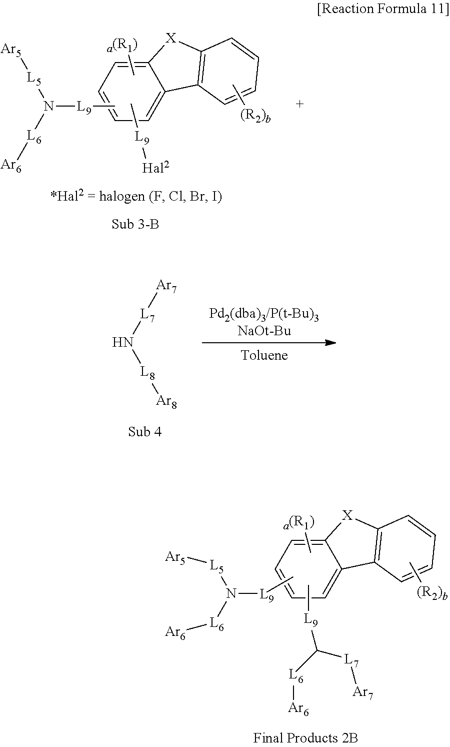

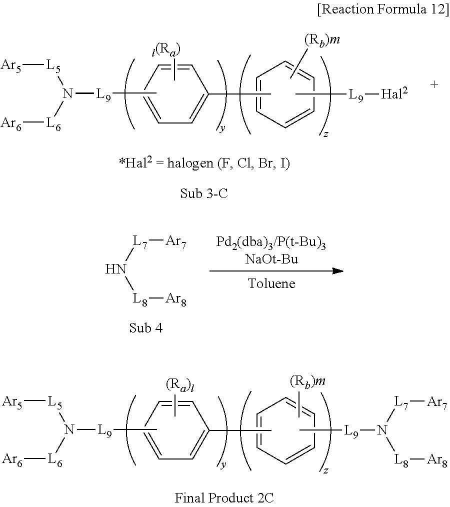

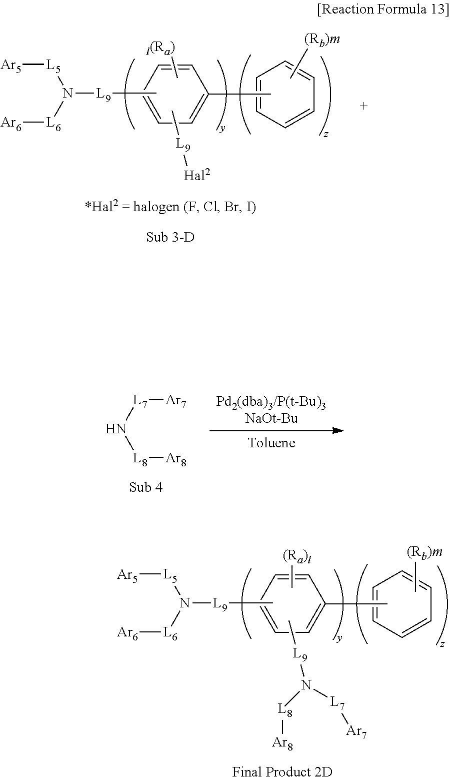

Synthesis of Sub 3-A and Sub 3-B

Sub 3-A and Sub 3-B of Reaction Formula 10 and Reaction Formula 11 may be synthesized through, but are not limited to, a reaction path of the following Reaction Formula 14 and Reaction Formula 15.

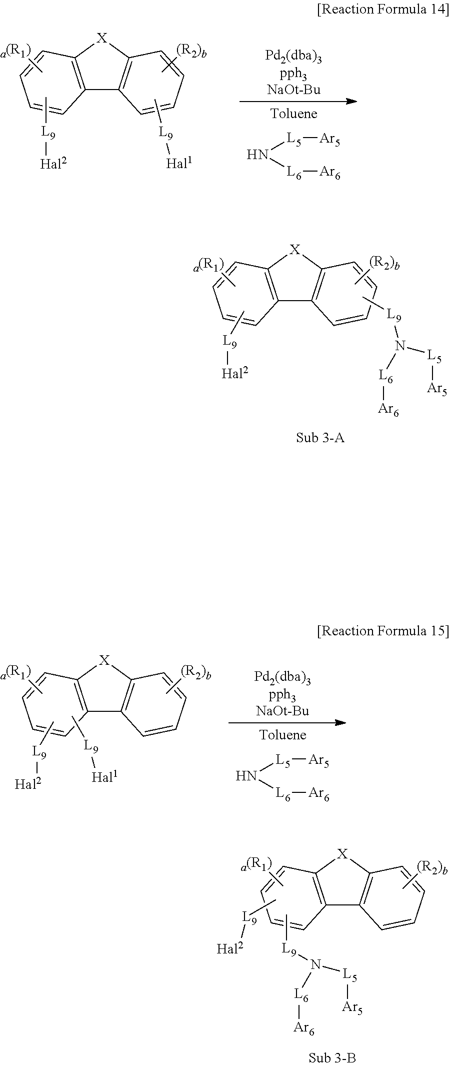

Synthesis of Sub 3-C and Sub 3-D

Sub 3-C and Sub 3-D of Reaction Formula 12 and Reaction Formula 13 may be synthesized through, but are not limited to, a reaction path of the following Reaction Formula 16 and Reaction Formula 17.

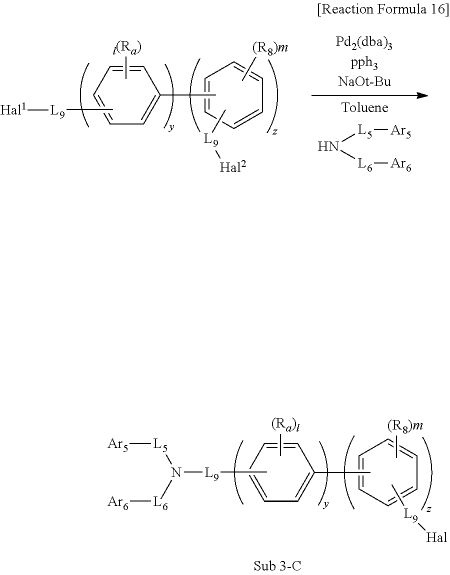

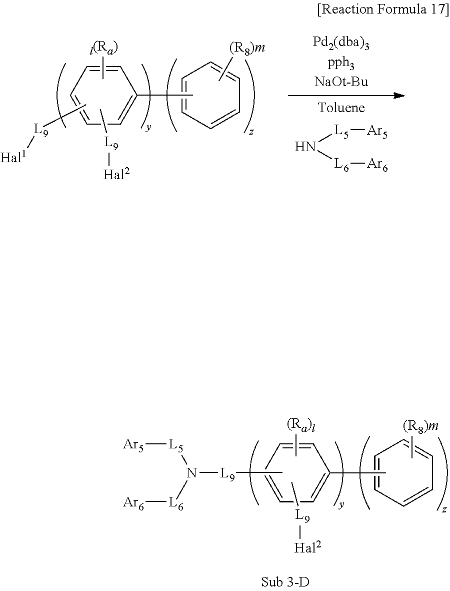

### 1. Sub 3-1 Synthesis Example

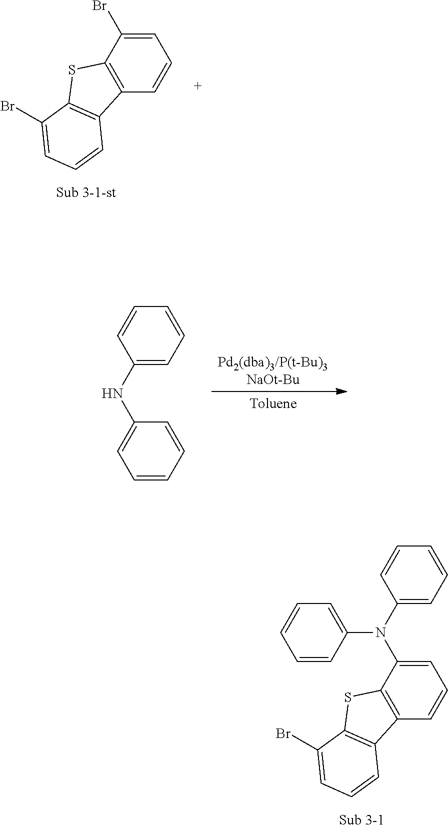 [SMILES: Brc1cccc2c1sc1c(Br)cccc12 ; c1ccc(Nc2ccccc2)cc1 ; "Pd2(dba)3/P(t-Bu)3NaOt-Bu" ; "Toluene" ; Brc1cccc2c1sc1c(N(c3ccccc3)c3ccccc3)cccc12]

After diphenylamine (15.22 g, 89.94 mmol) serving as a starting material was dissolved with toluene (750 ml) in a round bottom flask, Sub 3-1-st (CAS Registry Number: 669773-34-6) (46.14 g, 134.91 mmol), Pd₂(dba)₃ (2.47 g, 2.70 mmol), P(t-Bu)₃ (1.82 g, 8.99 mmol), and NaOt-Bu (25.93 g, 269.81 mmol) were added, followed by stirring at 0° C. When the reaction was completed, extraction was performed with CH₂Cl₂ and water, and an organic layer was dried with MgSO₄ and concentrated. Afterwards, a resultant compound was subjected to silica gel column chromatography and recrystallization, thereby creating a product 23.61 g (yield: 61%)).

### 2. Sub 3-63 Synthesis Example

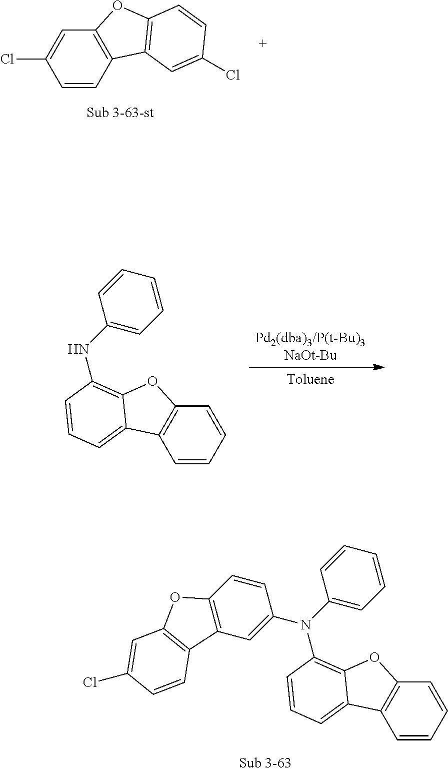 [SMILES: Clc1ccc2c(c1)oc1ccc(Cl)cc12 ; c1ccc(Nc2cccc3c2oc2ccccc23)cc1 ; "Pd2(dba)3/P(t-Bu)3NaOt-Bu" ; "Toluene" ; Clc1ccc2c(c1)oc1ccc(N(c3ccccc3)c3cccc4c3oc3ccccc34)cc12]

After sub 3-63-st (10 g, 42.18 mmol), Pd₂(dba)₃ (1.16 g, 1.27 mmol) P(t-Bu)₃ (8.53 g, 42.18 mmol), NaOt-Bu (8.11 g, 84.36 mmol), and toluene (422 ml) were added to N-phenyldibenzo[b,d]furan-4-amine (10.94 g, 42.18 mmol) serving as a starting material, a synthesis method of Sub 3-1 was used to create a product 13 g (yield: 67%).

### 3. Sub 3-157 Synthesis Example

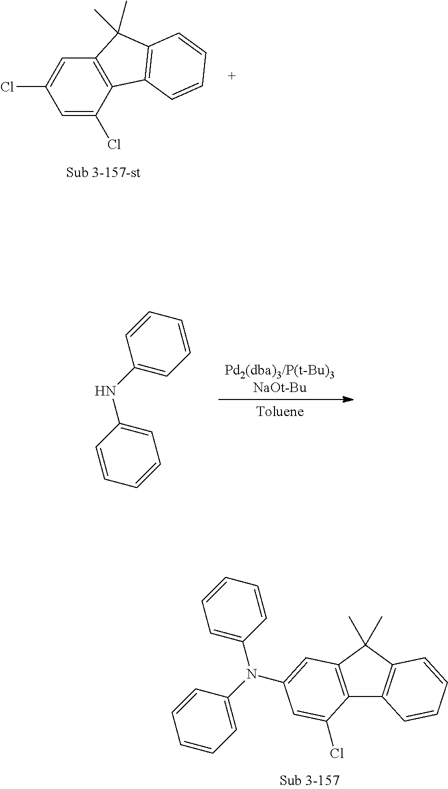 [SMILES: CC1(C)c2ccccc2-c2c(Cl)cc(Cl)cc21 ; c1ccc(Nc2ccccc2)cc1 ; "Pd2(dba)3/P(t-Bu)3NaOt-Bu" ; "Toluene" ; CC1(C)c2ccccc2-c2c(Cl)cc(N(c3ccccc3)c3ccccc3)cc21]

After Sub 3-157-st (16.08 g, 95 mmol), Pd₂(dba)₃ (2.61 g, 2.85 mmol) P(t-Bu)₃ (19.22 g, 95 mmol), NaOt-Bu (18.26 g, 190 mmol), and toluene (950 ml) were added to diphenylamine (25 g, 95 mmol) serving as a starting material, a synthesis method of Sub 3-1 was used to create a product 24.45 g (yield: 65%).

Sub 3-A to Sub 3-D may have, but are not limited to, the following examples.

TABLE 4

| | |
|---|---|
| 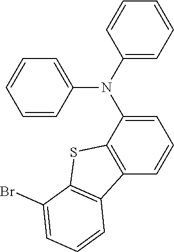 [SMILES: Brc1cccc2c1sc1c(N(c3ccccc3)c3ccccc3)cccc12] | Sub 3-1 |
| 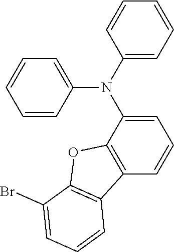 [SMILES: Brc1cccc2c1oc1c(N(c3ccccc3)c3ccccc3)cccc12] | Sub 3-2 |
| 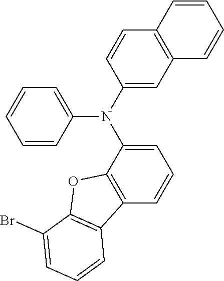 [SMILES: Brc1cccc2c1oc1c(N(c3ccccc3)c3ccc4ccccc4c3)cccc12] | Sub 3-3 |
| 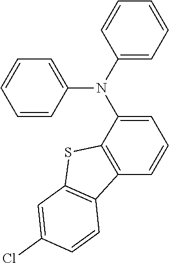 [SMILES: Clc1ccc2c(c1)sc1c(N(c3ccccc3)c3ccccc3)cccc12] | Sub 3-4 |
| 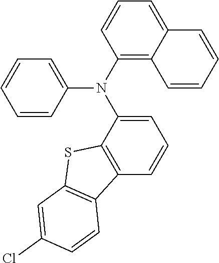 [SMILES: Clc1ccc2c(c1)sc1c(N(c3ccccc3)c3cccc4ccccc34)cccc12] | Sub 3-5 |
| 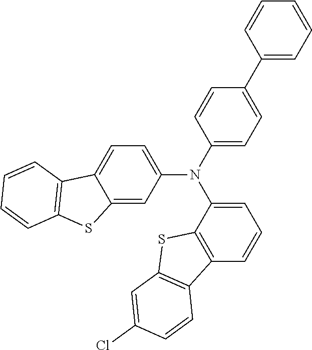 [SMILES: Clc1ccc2c(c1)sc1c(N(c3ccc(-c4ccccc4)cc3)c3ccc4c(c3)sc3ccccc34)cccc12] | Sub 3-6 |
| 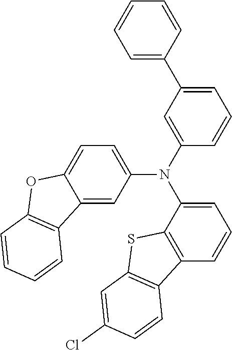 [SMILES: Clc1ccc2c(c1)sc1c(N(c3cccc(-c4ccccc4)c3)c3ccc4oc5ccccc5c4c3)cccc12] | Sub 3-7 |
| 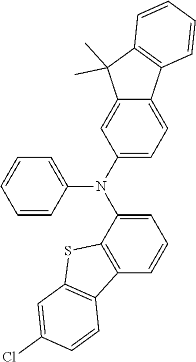 [SMILES: CC1(C)c2ccccc2-c2ccc(N(c3ccccc3)c3cccc4c3sc3cc(Cl)ccc34)cc21] | Sub 3-8 |
| 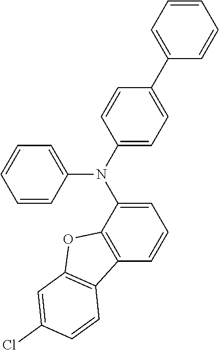 [SMILES: Clc1ccc2c(c1)oc1c(N(c3ccccc3)c3ccc(-c4ccccc4)cc3)cccc12] | Sub 3-9 |
| 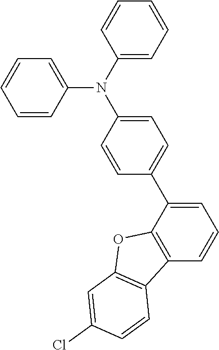 [SMILES: Clc1ccc2c(c1)oc1c(-c3ccc(N(c4ccccc4)c4ccccc4)cc3)cccc12] | Sub 3-10 |
| 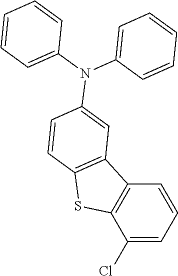 [SMILES: Clc1cccc2c1sc1ccc(N(c3ccccc3)c3ccccc3)cc12] | Sub 3-11 |
| 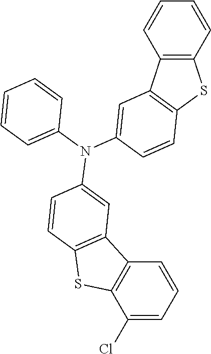 [SMILES: Clc1cccc2c1sc1ccc(N(c3ccccc3)c3ccc4sc5ccccc5c4c3)cc12] | Sub 3-12 |
| 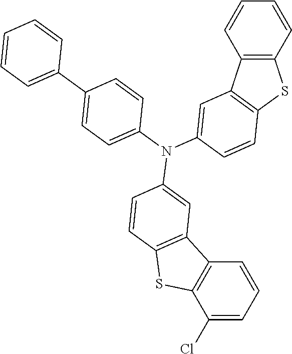 [SMILES: Clc1cccc2c1sc1ccc(N(c3ccc(-c4ccccc4)cc3)c3ccc4sc5ccccc5c4c3)cc12] | Sub 3-13 |
| 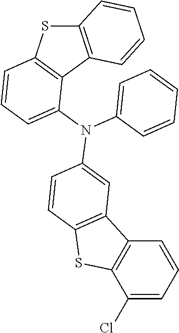 [SMILES: Clc1cccc2c1sc1ccc(N(c3ccccc3)c3cccc4sc5ccccc5c34)cc12] | Sub 3-14 |
| 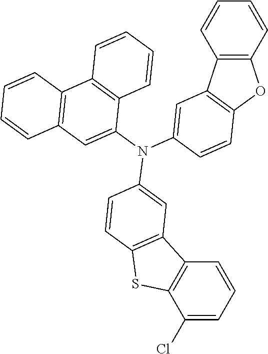 [SMILES: Clc1cccc2c1sc1ccc(N(c3ccc4oc5ccccc5c4c3)c3cc4ccccc4c4ccccc34)cc12] | Sub 3-15 |
| 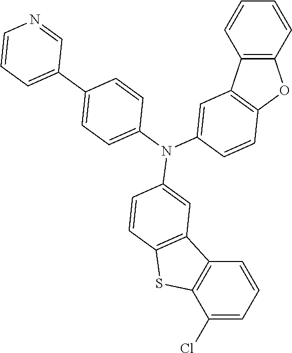 [SMILES: Clc1cccc2c1sc1ccc(N(c3ccc(-c4cccnc4)cc3)c3ccc4oc5ccccc5c4c3)cc12] | Sub 3-16 |
| 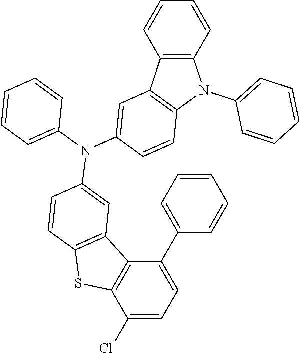 [SMILES: Clc1ccc(-c2ccccc2)c2c1sc1ccc(N(c3ccccc3)c3ccc4c(c3)c3ccccc3n4-c3ccccc3)cc12] | Sub 3-17 |
| 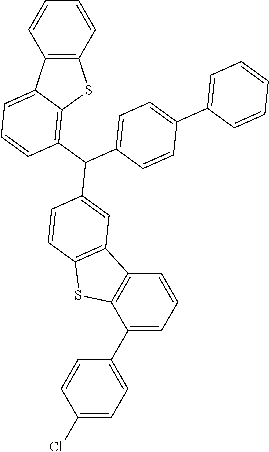 [SMILES: Clc1ccc(-c2cccc3c2sc2ccc(C(c4ccc(-c5ccccc5)cc4)c4cccc5c4sc4ccccc45)cc23)cc1] | Sub 3-18 |
| 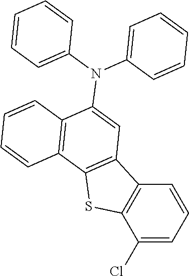 [SMILES: Clc1cccc2c1sc1c3ccccc3c(N(c3ccccc3)c3ccccc3)cc21] | Sub 3-19 |
| 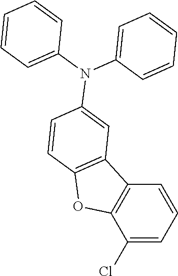 [SMILES: Clc1cccc2c1oc1ccc(N(c3ccccc3)c3ccccc3)cc12] | Sub 3-20 |
| 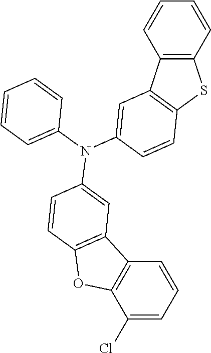 [SMILES: Clc1cccc2c1oc1ccc(N(c3ccccc3)c3ccc4sc5ccccc5c4c3)cc12] | Sub 3-21 |
| 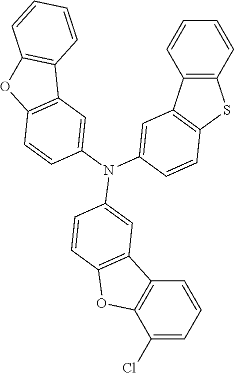 [SMILES: Clc1cccc2c1oc1ccc(N(c3ccc4oc5ccccc5c4c3)c3ccc4sc5ccccc5c4c3)cc12] | Sub 3-22 |
| 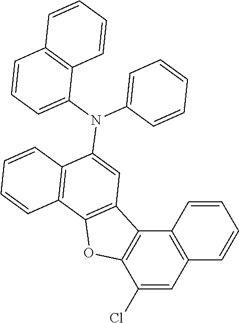 [SMILES: Clc1cc2ccccc2c2c1oc1c3ccccc3c(N(c3ccccc3)c3cccc4ccccc34)cc12] | Sub 3-23 |
| 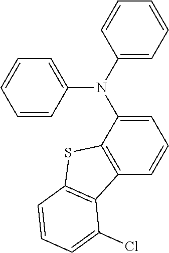 [SMILES: Clc1cccc2sc3c(N(c4ccccc4)c4ccccc4)cccc3c12] | Sub 3-24 |
| 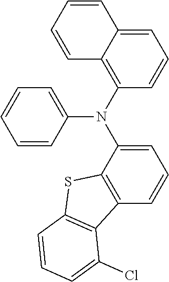 [SMILES: Clc1cccc2sc3c(N(c4ccccc4)c4cccc5ccccc45)cccc3c12] | Sub 3-25 |
| 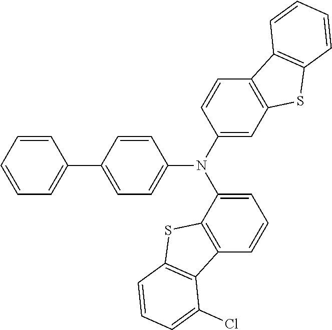 [SMILES: Clc1cccc2sc3c(N(c4ccc(-c5ccccc5)cc4)c4ccc5c(c4)sc4ccccc45)cccc3c12] | Sub 3-26 |
| 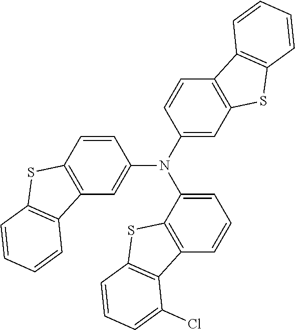 [SMILES: Clc1cccc2sc3c(N(c4ccc5c(c4)sc4ccccc45)c4ccc5sc6ccccc6c5c4)cccc3c12] | Sub 3-27 |
| 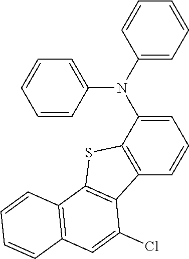 [SMILES: Clc1cc2ccccc2c2sc3c(N(c4ccccc4)c4ccccc4)cccc3c12] | Sub 3-28 |
| 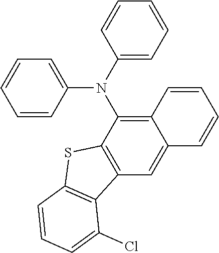 [SMILES: Clc1cccc2sc3c(N(c4ccccc4)c4ccccc4)c4ccccc4cc3c12] | Sub 3-29 |
| 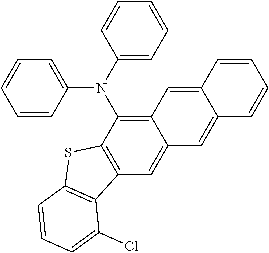 [SMILES: Clc1cccc2sc3c(N(c4ccccc4)c4ccccc4)c4cc5ccccc5cc4cc3c12] | Sub 3-30 |
| 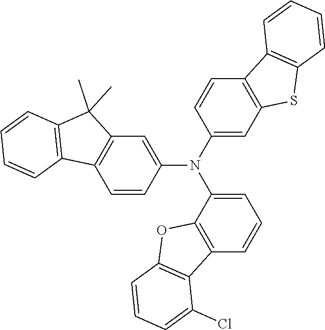 [SMILES: CC1(C)c2ccccc2-c2ccc(N(c3ccc4c(c3)sc3ccccc34)c3cccc4c3oc3cccc(Cl)c34)cc21] | Sub 3-31 |
| 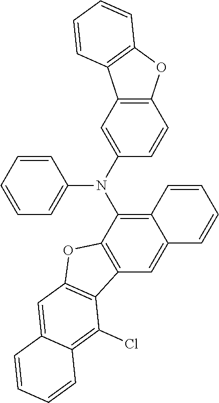 [SMILES: Clc1c2ccccc2cc2oc3c(N(c4ccccc4)c4ccc5oc6ccccc6c5c4)c4ccccc4cc3c12] | Sub 3-32 |
| 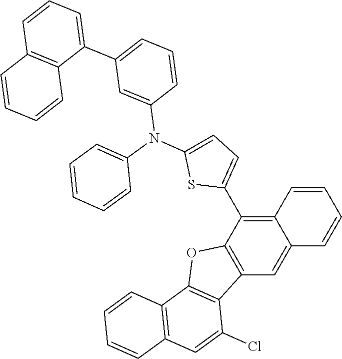 [SMILES: Clc1cc2ccccc2c2oc3c(-c4ccc(N(c5ccccc5)c5cccc(-c6cccc7ccccc67)c5)s4)c4ccccc4cc3c12] | Sub 3-33 |
| 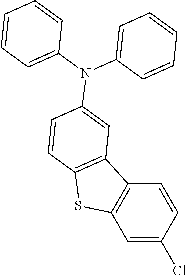 [SMILES: Clc1ccc2c(c1)sc1ccc(N(c3ccccc3)c3ccccc3)cc12] | Sub 3-34 |
| 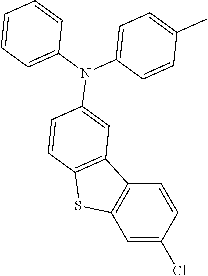 [SMILES: Cc1ccc(N(c2ccccc2)c2ccc3sc4cc(Cl)ccc4c3c2)cc1] | Sub 3-35 |
| 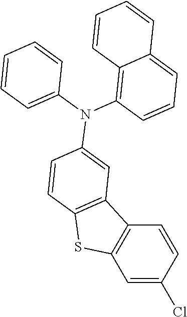 [SMILES: Clc1ccc2c(c1)sc1ccc(N(c3ccccc3)c3cccc4ccccc34)cc12] | Sub 3-36 |
| 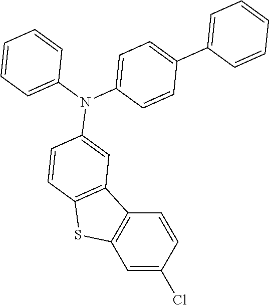 [SMILES: Clc1ccc2c(c1)sc1ccc(N(c3ccccc3)c3ccc(-c4ccccc4)cc3)cc12] | Sub 3-37 |
| 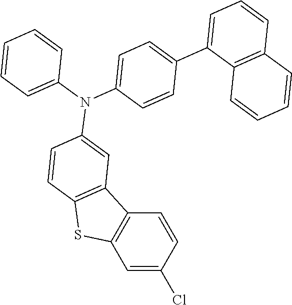 [SMILES: Clc1ccc2c(c1)sc1ccc(N(c3ccccc3)c3ccc(-c4cccc5ccccc45)cc3)cc12] | Sub 3-38 |
| 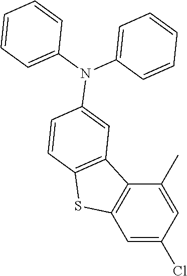 [SMILES: Cc1cc(Cl)cc2sc3ccc(N(c4ccccc4)c4ccccc4)cc3c12] | Sub 3-39 |
| 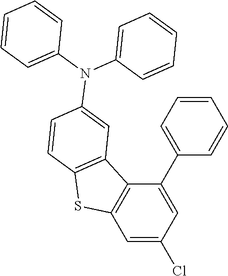 [SMILES: Clc1cc(-c2ccccc2)c2c(c1)sc1ccc(N(c3ccccc3)c3ccccc3)cc12] | Sub 3-40 |
| 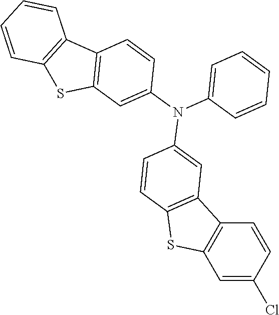 [SMILES: Clc1ccc2c(c1)sc1ccc(N(c3ccccc3)c3ccc4c(c3)sc3ccccc34)cc12] | Sub 3-41 |
| 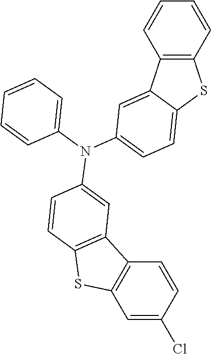 [SMILES: Clc1ccc2c(c1)sc1ccc(N(c3ccccc3)c3ccc4sc5ccccc5c4c3)cc12] | Sub 3-42 |
| 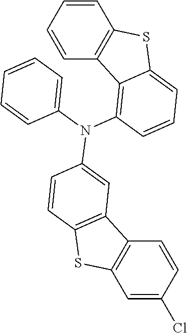 [SMILES: Clc1ccc2c(c1)sc1ccc(N(c3ccccc3)c3cccc4sc5ccccc5c34)cc12] | Sub 3-43 |
| 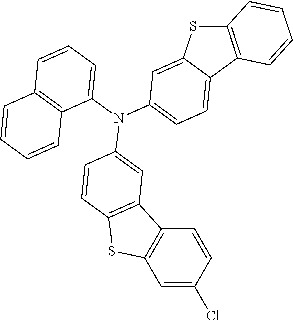 [SMILES: Clc1ccc2c(c1)sc1ccc(N(c3ccc4c(c3)sc3ccccc34)c3cccc4ccccc34)cc12] | Sub 3-44 |
| 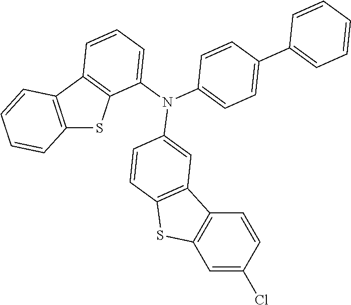 [SMILES: Clc1ccc2c(c1)sc1ccc(N(c3ccc(-c4ccccc4)cc3)c3cccc4c3sc3ccccc34)cc12] | Sub 3-45 |
| 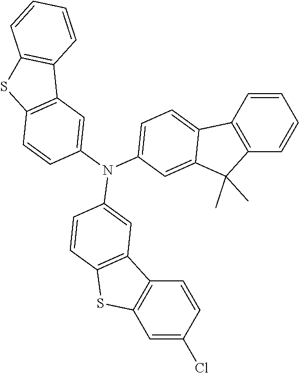 [SMILES: CC1(C)c2ccccc2-c2ccc(N(c3ccc4sc5ccccc5c4c3)c3ccc4sc5cc(Cl)ccc5c4c3)cc21] | Sub 3-46 |
| 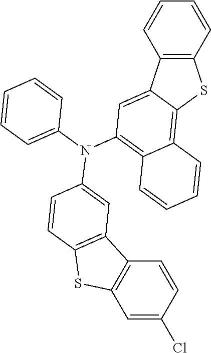 [SMILES: Clc1ccc2c(c1)sc1ccc(N(c3ccccc3)c3cc4c5ccccc5sc4c4ccccc34)cc12] | Sub 3-47 |
| 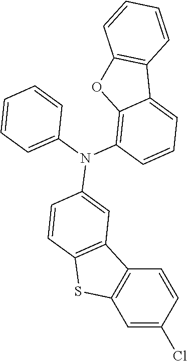 [SMILES: Clc1ccc2c(c1)sc1ccc(N(c3ccccc3)c3cccc4c3oc3ccccc34)cc12] | Sub 3-48 |
| 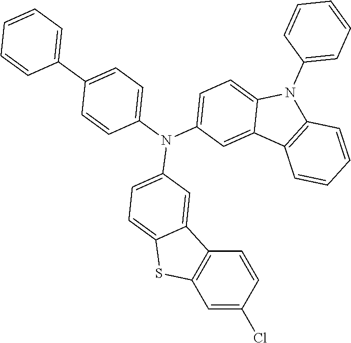 [SMILES: Clc1ccc2c(c1)sc1ccc(N(c3ccc(-c4ccccc4)cc3)c3ccc4c(c3)c3ccccc3n4-c3ccccc3)cc12] | Sub 3-49 |
| 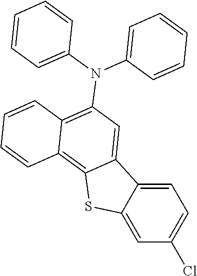 [SMILES: Clc1ccc2c(c1)sc1c3ccccc3c(N(c3ccccc3)c3ccccc3)cc21] | Sub 3-50 |
| 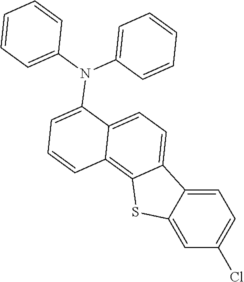 [SMILES: Clc1ccc2c(c1)sc1c3cccc(N(c4ccccc4)c4ccccc4)c3ccc21] | Sub 3-51 |
| 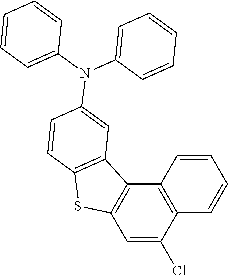 [SMILES: Clc1cc2sc3ccc(N(c4ccccc4)c4ccccc4)cc3c2c2ccccc12] | Sub 3-52 |
| 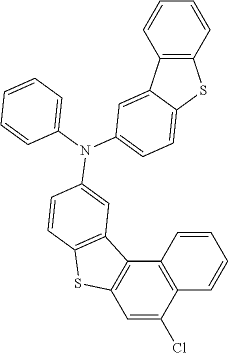 [SMILES: Clc1cc2sc3ccc(N(c4ccccc4)c4ccc5sc6ccccc6c5c4)cc3c2c2ccccc12] | Sub 3-53 |
| 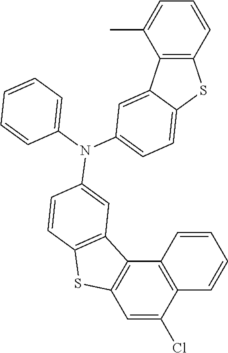 [SMILES: Cc1cccc2sc3ccc(N(c4ccccc4)c4ccc5sc6cc(Cl)c7ccccc7c6c5c4)cc3c12] | Sub 3-54 |
| 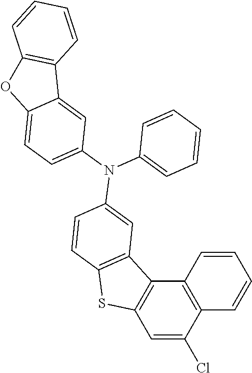 [SMILES: Clc1cc2sc3ccc(N(c4ccccc4)c4ccc5oc6ccccc6c5c4)cc3c2c2ccccc12] | Sub 3-55 |
| 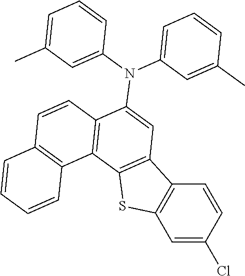 [SMILES: Cc1cccc(N(c2cccc(C)c2)c2cc3c4ccc(Cl)cc4sc3c3c2ccc2ccccc23)c1] | Sub 3-56 |
| 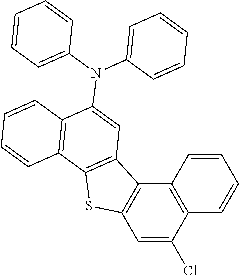 [SMILES: Clc1cc2sc3c4ccccc4c(N(c4ccccc4)c4ccccc4)cc3c2c2ccccc12] | Sub 3-57 |
| 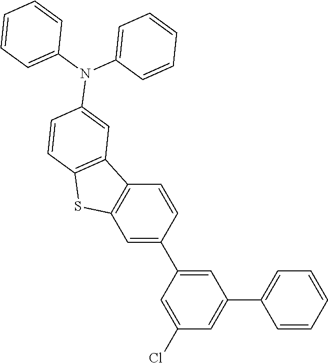 [SMILES: Clc1cc(-c2ccccc2)cc(-c2ccc3c(c2)sc2ccc(N(c4ccccc4)c4ccccc4)cc23)c1] | Sub 3-58 |
| 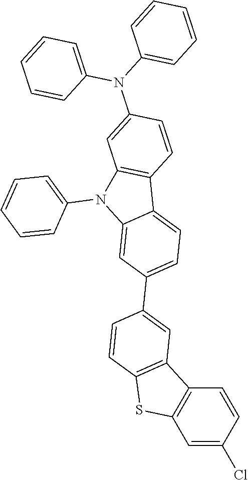 [SMILES: Clc1ccc2c(c1)sc1ccc(-c3ccc4c5ccc(N(c6ccccc6)c6ccccc6)cc5n(-c5ccccc5)c4c3)cc12] | Sub 3-59 |
| 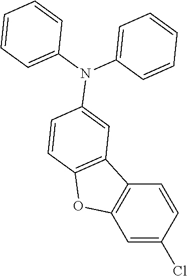 [SMILES: Clc1ccc2c(c1)oc1ccc(N(c3ccccc3)c3ccccc3)cc12] | Sub 3-60 |
| 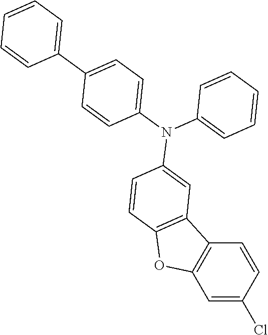 [SMILES: Clc1ccc2c(c1)oc1ccc(N(c3ccccc3)c3ccc(-c4ccccc4)cc3)cc12] | Sub 3-61 |
| 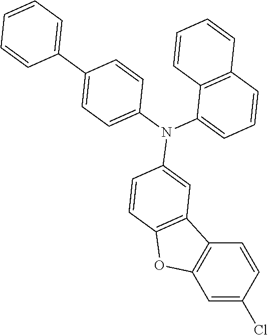 [SMILES: Clc1ccc2c(c1)oc1ccc(N(c3ccc(-c4ccccc4)cc3)c3cccc4ccccc34)cc12] | Sub 3-62 |
| 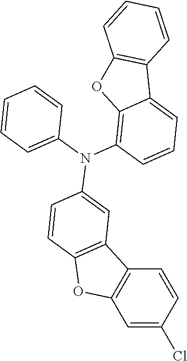 [SMILES: Clc1ccc2c(c1)oc1ccc(N(c3ccccc3)c3cccc4c3oc3ccccc34)cc12] | Sub 3-63 |
| 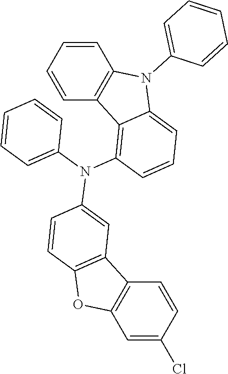 [SMILES: Clc1ccc2c(c1)oc1ccc(N(c3ccccc3)c3cccc4c3c3ccccc3n4-c3ccccc3)cc12] | Sub 3-64 |
| 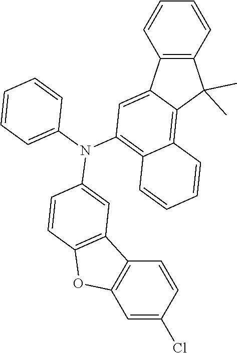 [SMILES: CC1(C)c2ccccc2-c2cc(N(c3ccccc3)c3ccc4oc5cc(Cl)ccc5c4c3)c3ccccc3c21] | Sub 3-65 |
| 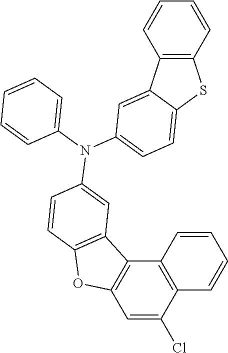 [SMILES: Clc1cc2oc3ccc(N(c4ccccc4)c4ccc5sc6ccccc6c5c4)cc3c2c2ccccc12] | Sub 3-66 |
| 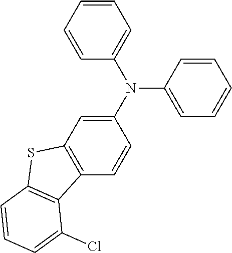 [SMILES: Clc1cccc2sc3cc(N(c4ccccc4)c4ccccc4)ccc3c12] | Sub 3-67 |
| 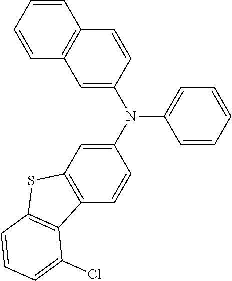 [SMILES: Clc1cccc2sc3cc(N(c4ccccc4)c4ccc5ccccc5c4)ccc3c12] | Sub 3-68 |
| 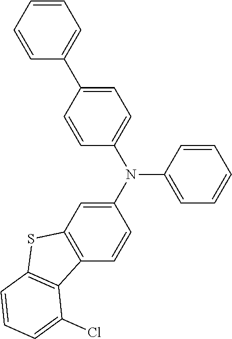 [SMILES: Clc1cccc2sc3cc(N(c4ccccc4)c4ccc(-c5ccccc5)cc4)ccc3c12] | Sub 3-69 |
| 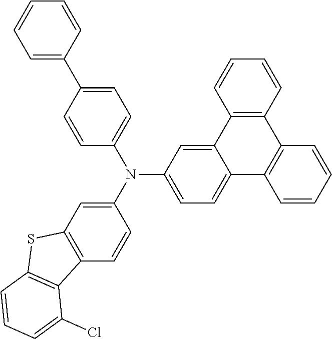 [SMILES: Clc1cccc2sc3cc(N(c4ccc(-c5ccccc5)cc4)c4ccc5c6ccccc6c6ccccc6c5c4)ccc3c12] | Sub 3-70 |
| 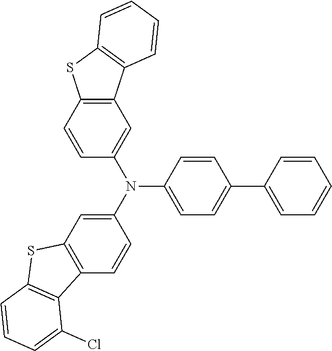 [SMILES: Clc1cccc2sc3cc(N(c4ccc(-c5ccccc5)cc4)c4ccc5sc6ccccc6c5c4)ccc3c12] | Sub 3-71 |
| 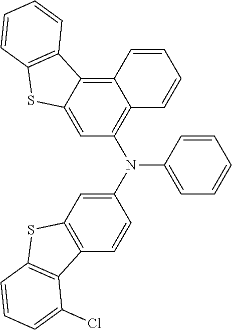 [SMILES: Clc1cccc2sc3cc(N(c4ccccc4)c4cc5sc6ccccc6c5c5ccccc45)ccc3c12] | Sub 3-72 |
| 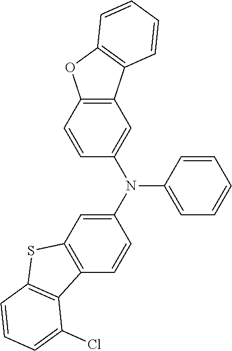 [SMILES: Clc1cccc2sc3cc(N(c4ccccc4)c4ccc5oc6ccccc6c5c4)ccc3c12] | Sub 3-73 |
| 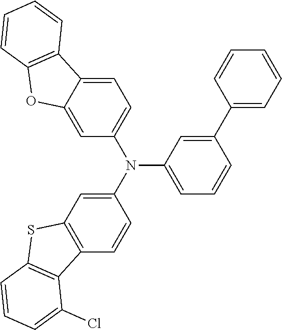 [SMILES: Clc1cccc2sc3cc(N(c4cccc(-c5ccccc5)c4)c4ccc5c(c4)oc4ccccc45)ccc3c12] | Sub 3-74 |
| 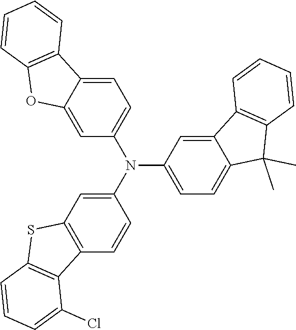 [SMILES: CC1(C)c2ccccc2-c2cc(N(c3ccc4c(c3)oc3ccccc34)c3ccc4c(c3)sc3cccc(Cl)c34)ccc21] | Sub 3-75 |
| 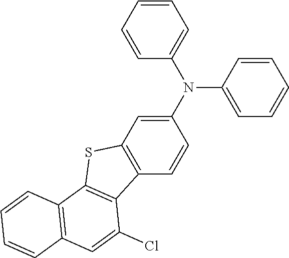 [SMILES: Clc1cc2ccccc2c2sc3cc(N(c4ccccc4)c4ccccc4)ccc3c12] | Sub 3-76 |
| 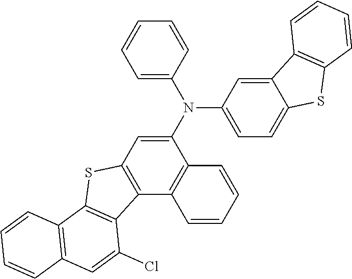 [SMILES: Clc1cc2ccccc2c2sc3cc(N(c4ccccc4)c4ccc5sc6ccccc6c5c4)c4ccccc4c3c12] | Sub 3-77 |
| 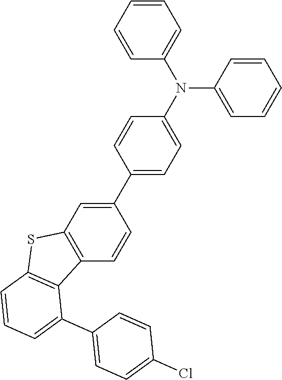 [SMILES: Clc1ccc(-c2cccc3sc4cc(-c5ccc(N(c6ccccc6)c6ccccc6)cc5)ccc4c23)cc1] | Sub 3-78 |
| 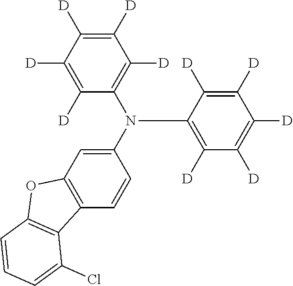 [SMILES: [2H]c1c([2H])c([2H])c(N(c2ccc3c(c2)oc2cccc(Cl)c23)c2c([2H])c([2H])c([2H])c([2H])c2[2H])c([2H])c1[2H]] | Sub 3-79 |
| 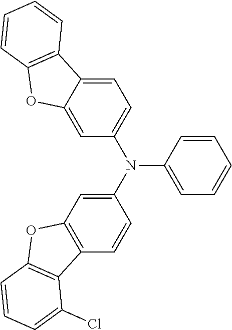 [SMILES: Clc1cccc2oc3cc(N(c4ccccc4)c4ccc5c(c4)oc4ccccc45)ccc3c12] | Sub 3-80 |
| 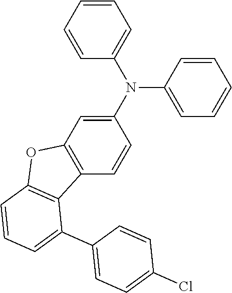 [SMILES: Clc1ccc(-c2cccc3oc4cc(N(c5ccccc5)c5ccccc5)ccc4c23)cc1] | Sub 3-81 |
| 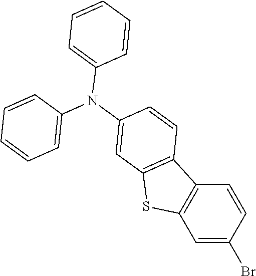 [SMILES: Brc1ccc2c(c1)sc1cc(N(c3ccccc3)c3ccccc3)ccc12] | Sub 3-82 |
| 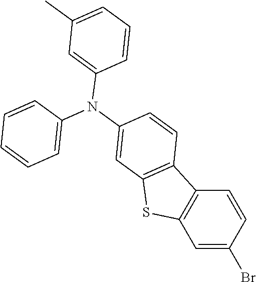 [SMILES: Cc1cccc(N(c2ccccc2)c2ccc3c(c2)sc2cc(Br)ccc23)c1] | Sub 3-83 |
| 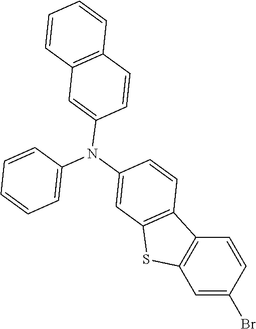 [SMILES: Brc1ccc2c(c1)sc1cc(N(c3ccccc3)c3ccc4ccccc4c3)ccc12] | Sub 3-84 |
| 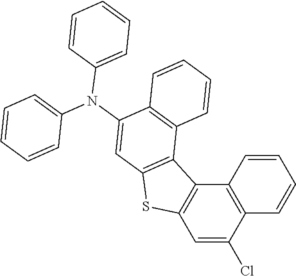 [SMILES: Clc1cc2sc3cc(N(c4ccccc4)c4ccccc4)c4ccccc4c3c2c2ccccc12] | Sub 3-85 |
| 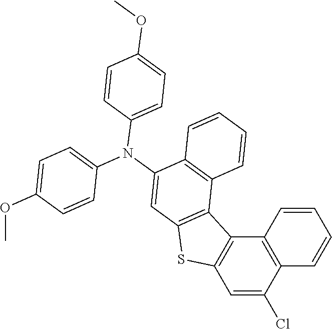 [SMILES: COc1ccc(N(c2ccc(OC)cc2)c2cc3sc4cc(Cl)c5ccccc5c4c3c3ccccc23)cc1] | Sub 3-86 |
| 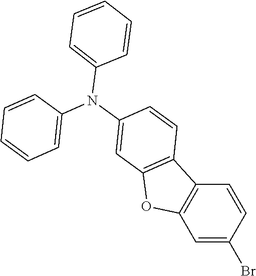 [SMILES: Brc1ccc2c(c1)oc1cc(N(c3ccccc3)c3ccccc3)ccc12] | Sub 3-87 |
| 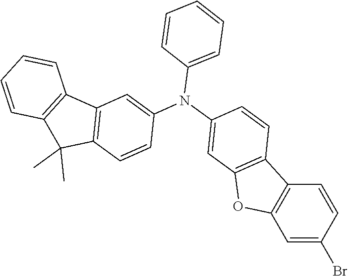 [SMILES: CC1(C)c2ccccc2-c2cc(N(c3ccccc3)c3ccc4c(c3)oc3cc(Br)ccc34)ccc21] | Sub 3-88 |
| 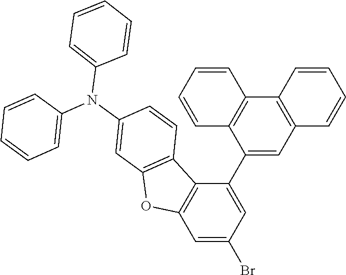 [SMILES: Brc1cc(-c2cc3ccccc3c3ccccc23)c2c(c1)oc1cc(N(c3ccccc3)c3ccccc3)ccc12] | Sub 3-89 |
| 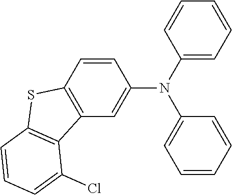 [SMILES: Clc1cccc2sc3ccc(N(c4ccccc4)c4ccccc4)cc3c12] | Sub 3-90 |
| 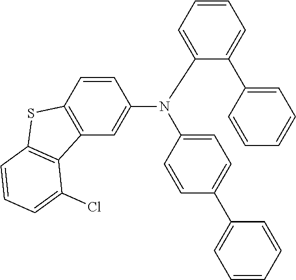 [SMILES: Clc1cccc2sc3ccc(N(c4ccc(-c5ccccc5)cc4)c4ccccc4-c4ccccc4)cc3c12] | Sub 3-91 |
| 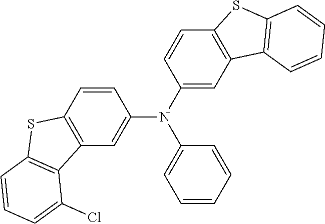 [SMILES: Clc1cccc2sc3ccc(N(c4ccccc4)c4ccc5sc6ccccc6c5c4)cc3c12] | Sub 3-92 |
| 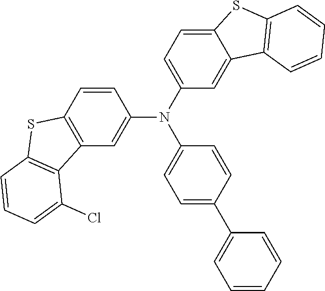 [SMILES: Clc1cccc2sc3ccc(N(c4ccc(-c5ccccc5)cc4)c4ccc5sc6ccccc6c5c4)cc3c12] | Sub 3-93 |
| 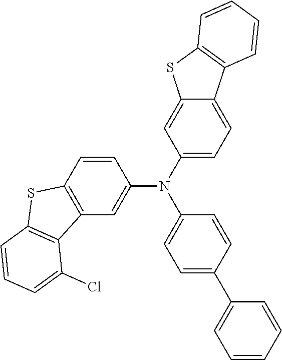 [SMILES: Clc1cccc2sc3ccc(N(c4ccc(-c5ccccc5)cc4)c4ccc5c(c4)sc4ccccc45)cc3c12] | Sub 3-94 |
| 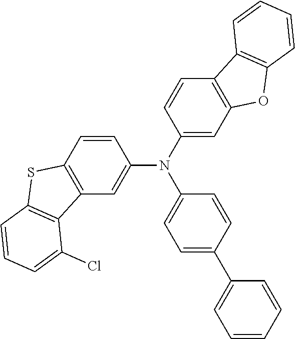 [SMILES: Clc1cccc2sc3ccc(N(c4ccc(-c5ccccc5)cc4)c4ccc5c(c4)oc4ccccc45)cc3c12] | Sub 3-95 |
| 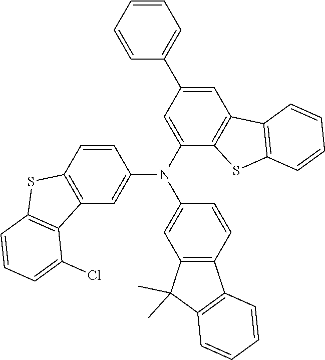 [SMILES: CC1(C)c2ccccc2-c2ccc(N(c3ccc4sc5cccc(Cl)c5c4c3)c3cc(-c4ccccc4)cc4c3sc3ccccc34)cc21] | Sub 3-96 |
| 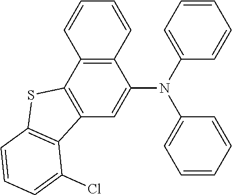 [SMILES: Clc1cccc2sc3c4ccccc4c(N(c4ccccc4)c4ccccc4)cc3c12] | Sub 3-97 |
| 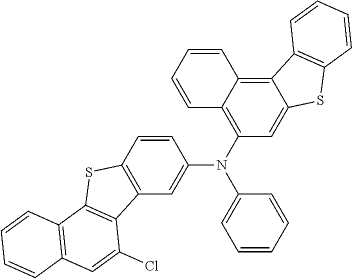 [SMILES: Clc1cc2ccccc2c2sc3ccc(N(c4ccccc4)c4cc5sc6ccccc6c5c5ccccc45)cc3c12] | Sub 3-98 |
| 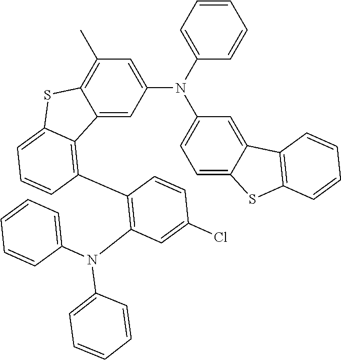 [SMILES: Cc1cc(N(c2ccccc2)c2ccc3sc4ccccc4c3c2)cc2c1sc1cccc(-c3ccc(Cl)cc3N(c3ccccc3)c3ccccc3)c12] | Sub 3-99 |
| 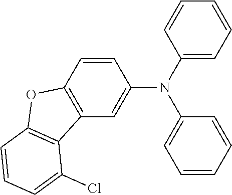 [SMILES: Clc1cccc2oc3ccc(N(c4ccccc4)c4ccccc4)cc3c12] | Sub 3-100 |
| 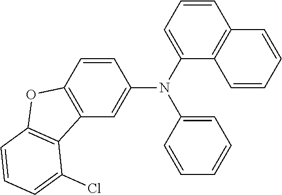 [SMILES: Clc1cccc2oc3ccc(N(c4ccccc4)c4cccc5ccccc45)cc3c12] | Sub 3-101 |
| 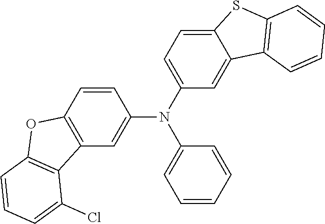 [SMILES: Clc1cccc2oc3ccc(N(c4ccccc4)c4ccc5sc6ccccc6c5c4)cc3c12] | Sub 3-102 |
| 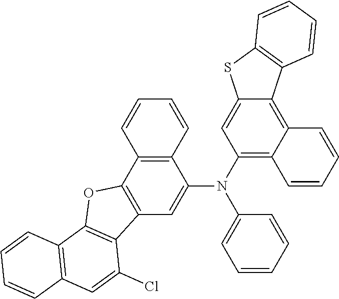 [SMILES: Clc1cc2ccccc2c2oc3c4ccccc4c(N(c4ccccc4)c4cc5sc6ccccc6c5c5ccccc45)cc3c12] | Sub 3-103 |
| 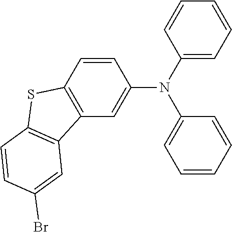 [SMILES: Brc1ccc2sc3ccc(N(c4ccccc4)c4ccccc4)cc3c2c1] | Sub 3-104 |
| 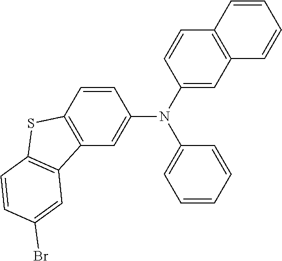 [SMILES: Brc1ccc2sc3ccc(N(c4ccccc4)c4ccc5ccccc5c4)cc3c2c1] | Sub 3-105 |
| 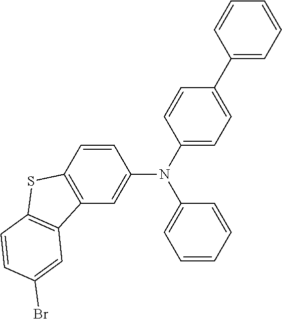 [SMILES: Brc1ccc2sc3ccc(N(c4ccccc4)c4ccc(-c5ccccc5)cc4)cc3c2c1] | Sub 3-106 |
| 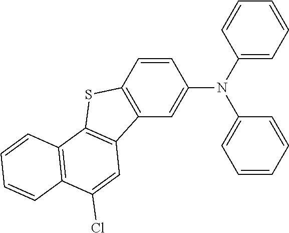 [SMILES: Clc1cc2c3cc(N(c4ccccc4)c4ccccc4)ccc3sc2c2ccccc12] | Sub 3-107 |
| 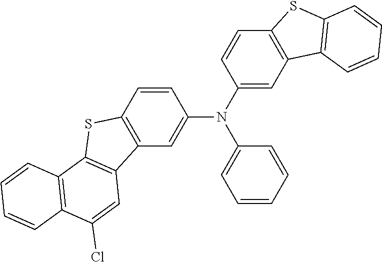 [SMILES: Clc1cc2c3cc(N(c4ccccc4)c4ccc5sc6ccccc6c5c4)ccc3sc2c2ccccc12] | Sub 3-108 |
| 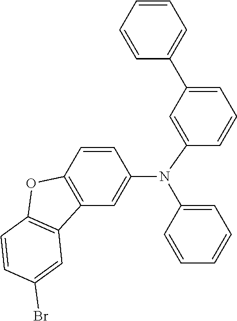 [SMILES: Brc1ccc2oc3ccc(N(c4ccccc4)c4cccc(-c5ccccc5)c4)cc3c2c1] | Sub 3-109 |
| 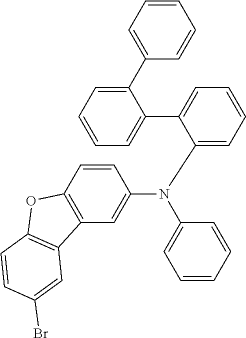 [SMILES: Brc1ccc2oc3ccc(N(c4ccccc4)c4ccccc4-c4ccccc4-c4ccccc4)cc3c2c1] | Sub 3-110 |
| 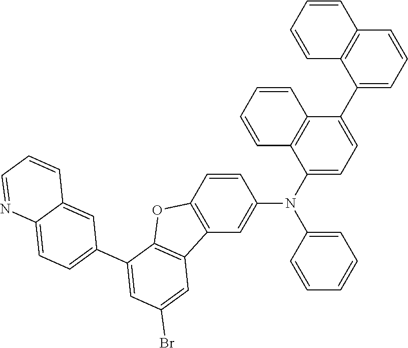 [SMILES: Brc1cc(-c2ccc3ncccc3c2)c2oc3ccc(N(c4ccccc4)c4ccc(-c5cccc6ccccc56)c5ccccc45)cc3c2c1] | Sub 3-111 |
| 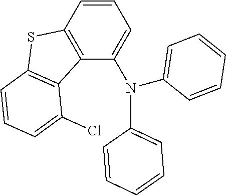 [SMILES: Clc1cccc2sc3cccc(N(c4ccccc4)c4ccccc4)c3c12] | Sub 3-112 |
| 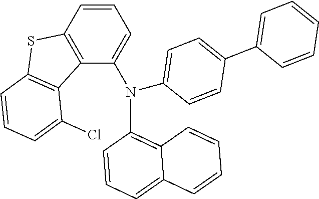 [SMILES: Clc1cccc2sc3cccc(N(c4ccc(-c5ccccc5)cc4)c4cccc5ccccc45)c3c12] | Sub 3-113 |
| 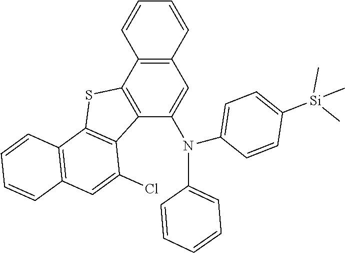 [SMILES: C[Si](C)(C)c1ccc(N(c2ccccc2)c2cc3ccccc3c3sc4c5ccccc5cc(Cl)c4c23)cc1] | Sub 3-114 |
| 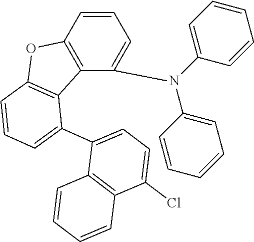 [SMILES: Clc1ccc(-c2cccc3oc4cccc(N(c5ccccc5)c5ccccc5)c4c23)c2ccccc12] | Sub 3-115 |
| 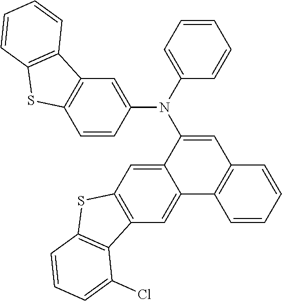 [SMILES: Clc1cccc2sc3cc4c(N(c5ccccc5)c5ccc6sc7ccccc7c6c5)cc5ccccc5c4cc3c12] | Sub 3-116 |
| 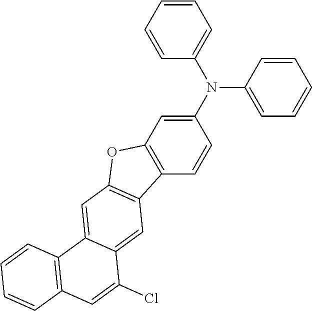 [SMILES: Clc1cc2ccccc2c2cc3oc4cc(N(c5ccccc5)c5ccccc5)ccc4c3cc12] | Sub 3-117 |
| 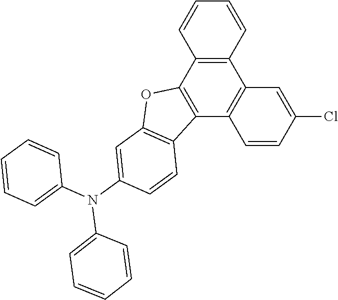 [SMILES: Clc1ccc2c(c1)c1ccccc1c1oc3cc(N(c4ccccc4)c4ccccc4)ccc3c21] | Sub 3-118 |
| 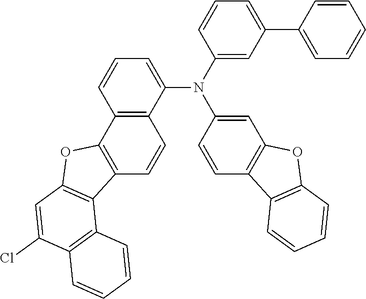 [SMILES: Clc1cc2oc3c4cccc(N(c5cccc(-c6ccccc6)c5)c5ccc6c(c5)oc5ccccc56)c4ccc3c2c2ccccc12] | Sub 3-119 |
| 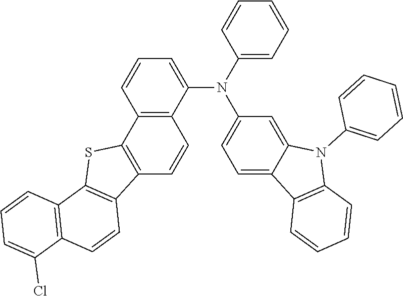 [SMILES: Clc1cccc2c1ccc1c3ccc4c(N(c5ccccc5)c5ccc6c7ccccc7n(-c7ccccc7)c6c5)cccc4c3sc21] | Sub 3-120 |
| 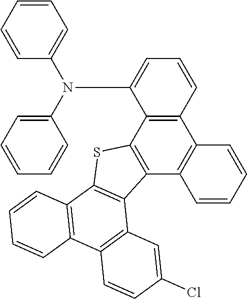 [SMILES: Clc1ccc2c3ccccc3c3sc4c5c(N(c6ccccc6)c6ccccc6)cccc5c5ccccc5c4c3c2c1] | Sub 3-121 |
| 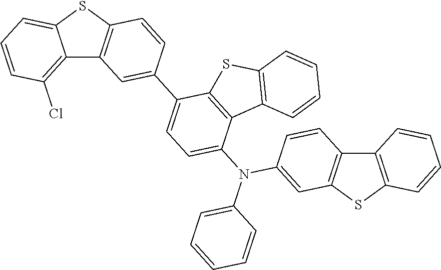 [SMILES: Clc1cccc2sc3ccc(-c4ccc(N(c5ccccc5)c5ccc6c(c5)sc5ccccc56)c5c4sc4ccccc45)cc3c12] | Sub 3-122 |
| 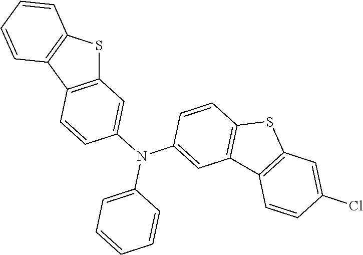 [SMILES: Clc1ccc2c(c1)sc1ccc(N(c3ccccc3)c3ccc4c(c3)sc3ccccc34)cc12] | Sub 3-123 |
| 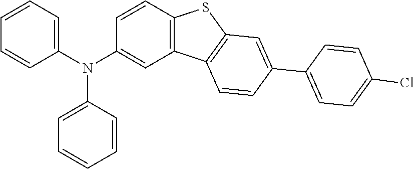 [SMILES: Clc1ccc(-c2ccc3c(c2)sc2ccc(N(c4ccccc4)c4ccccc4)cc23)cc1] | Sub 3-124 |
| 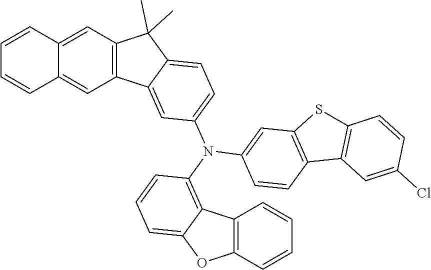 [SMILES: CC1(C)c2ccc(N(c3ccc4c(c3)sc3ccc(Cl)cc34)c3cccc4oc5ccccc5c34)cc2-c2cc3ccccc3cc21] | Sub 3-125 |
| 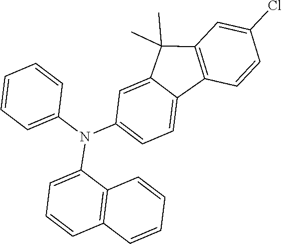 [SMILES: CC1(C)c2cc(Cl)ccc2-c2ccc(N(c3ccccc3)c3cccc4ccccc34)cc21] | Sub 3-126 |
| 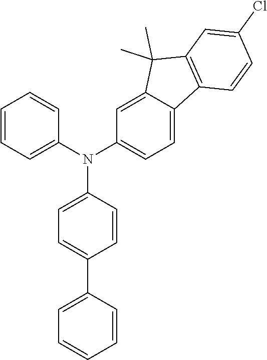 [SMILES: CC1(C)c2cc(Cl)ccc2-c2ccc(N(c3ccccc3)c3ccc(-c4ccccc4)cc3)cc21] | Sub 3-127 |
| 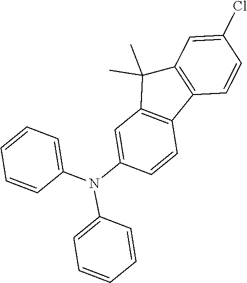 [SMILES: CC1(C)c2cc(Cl)ccc2-c2ccc(N(c3ccccc3)c3ccccc3)cc21] | Sub 3-128 |
| 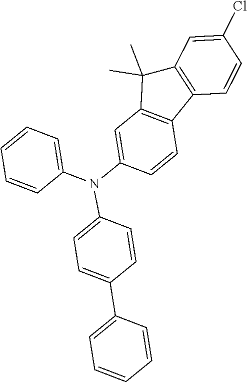 [SMILES: CC1(C)c2cc(Cl)ccc2-c2ccc(N(c3ccccc3)c3ccc(-c4ccccc4)cc3)cc21] | Sub 3-129 |
| 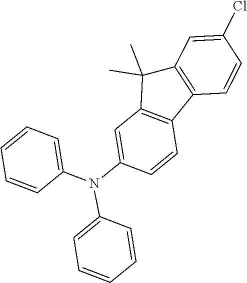 [SMILES: CC1(C)c2cc(Cl)ccc2-c2ccc(N(c3ccccc3)c3ccccc3)cc21] | Sub 3-130 |
| 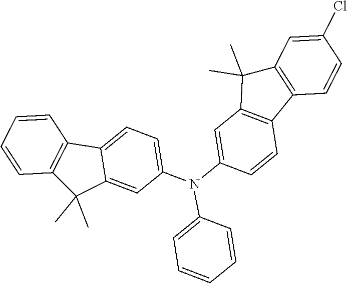 [SMILES: CC1(C)c2ccccc2-c2ccc(N(c3ccccc3)c3ccc4c(c3)C(C)(C)c3cc(Cl)ccc3-4)cc21] | Sub 3-131 |
| 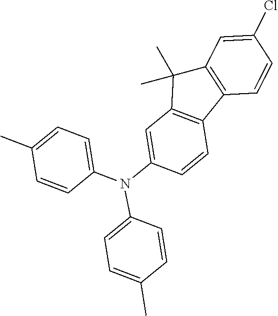 [SMILES: Cc1ccc(N(c2ccc(C)cc2)c2ccc3c(c2)C(C)(C)c2cc(Cl)ccc2-3)cc1] | Sub 3-132 |
| 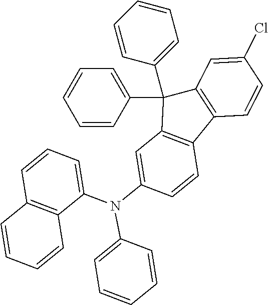 [SMILES: Clc1ccc2c(c1)C(c1ccccc1)(c1ccccc1)c1cc(N(c3ccccc3)c3cccc4ccccc34)ccc1-2] | Sub 3-133 |
| 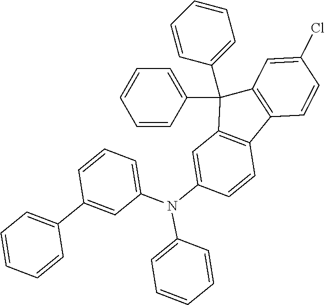 [SMILES: Clc1ccc2c(c1)C(c1ccccc1)(c1ccccc1)c1cc(N(c3ccccc3)c3cccc(-c4ccccc4)c3)ccc1-2] | Sub 3-134 |
| 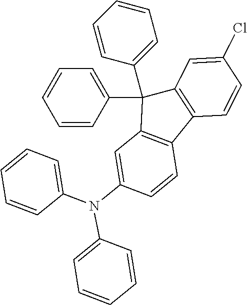 [SMILES: Clc1ccc2c(c1)C(c1ccccc1)(c1ccccc1)c1cc(N(c3ccccc3)c3ccccc3)ccc1-2] | Sub 3-135 |
| 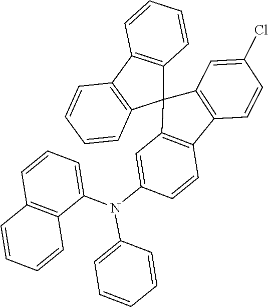 [SMILES: Clc1ccc2c(c1)C1(c3ccccc3-c3ccccc31)c1cc(N(c3ccccc3)c3cccc4ccccc34)ccc1-2] | Sub 3-136 |
| 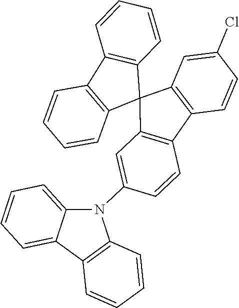 [SMILES: Clc1ccc2c(c1)C1(c3ccccc3-c3ccccc31)c1cc(-n3c4ccccc4c4ccccc43)ccc1-2] | Sub 3-137 |
| 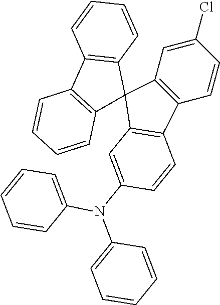 [SMILES: Clc1ccc2c(c1)C1(c3ccccc3-c3ccccc31)c1cc(N(c3ccccc3)c3ccccc3)ccc1-2] | Sub 3-138 |
| 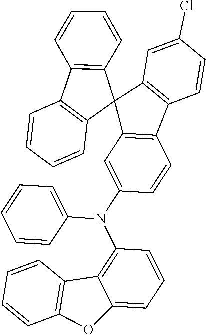 [SMILES: Clc1ccc2c(c1)C1(c3ccccc3-c3ccccc31)c1cc(N(c3ccccc3)c3cccc4oc5ccccc5c34)ccc1-2] | Sub 3-139 |
| 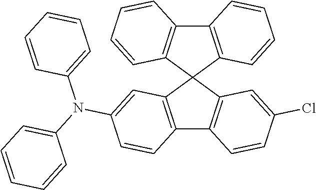 [SMILES: Clc1ccc2c(c1)C1(c3ccccc3-c3ccccc31)c1cc(N(c3ccccc3)c3ccccc3)ccc1-2] | Sub 3-140 |
| 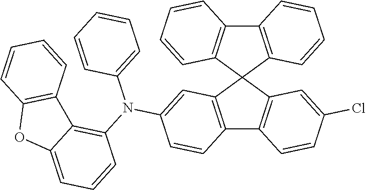 [SMILES: Clc1ccc2c(c1)C1(c3ccccc3-c3ccccc31)c1cc(N(c3ccccc3)c3cccc4oc5ccccc5c34)ccc1-2] | Sub 3-141 |
| 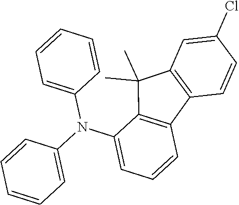 [SMILES: CC1(C)c2cc(Cl)ccc2-c2cccc(N(c3ccccc3)c3ccccc3)c21] | Sub 3-142 |
| 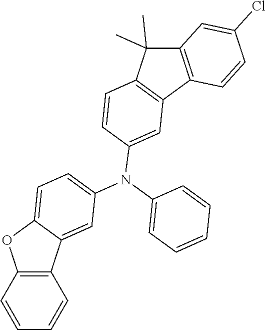 [SMILES: CC1(C)c2ccc(N(c3ccccc3)c3ccc4oc5ccccc5c4c3)cc2-c2ccc(Cl)cc21] | Sub 3-143 |
| 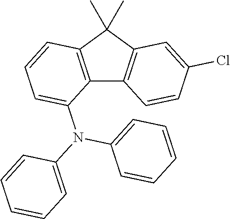 [SMILES: CC1(C)c2cc(Cl)ccc2-c2c(N(c3ccccc3)c3ccccc3)cccc21] | Sub 3-144 |
| 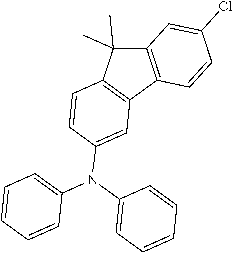 [SMILES: CC1(C)c2ccc(N(c3ccccc3)c3ccccc3)cc2-c2ccc(Cl)cc21] | Sub 3-145 |
| 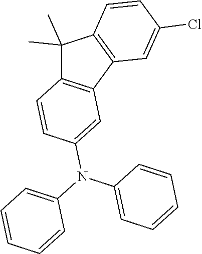 [SMILES: CC1(C)c2ccc(Cl)cc2-c2cc(N(c3ccccc3)c3ccccc3)ccc21] | Sub 3-146 |
| 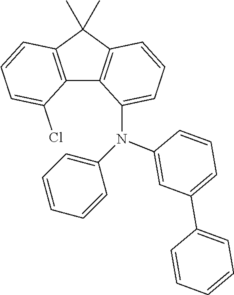 [SMILES: CC1(C)c2cccc(Cl)c2-c2c(N(c3ccccc3)c3cccc(-c4ccccc4)c3)cccc21] | Sub 3-147 |
| 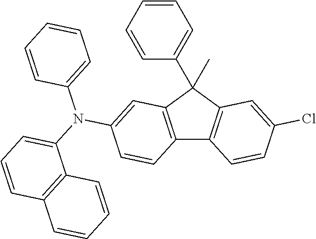 [SMILES: CC1(c2ccccc2)c2cc(Cl)ccc2-c2ccc(N(c3ccccc3)c3cccc4ccccc34)cc21] | Sub 3-148 |
| 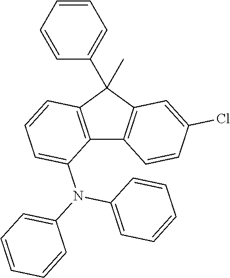 [SMILES: CC1(c2ccccc2)c2cc(Cl)ccc2-c2c(N(c3ccccc3)c3ccccc3)cccc21] | Sub 3-149 |
| 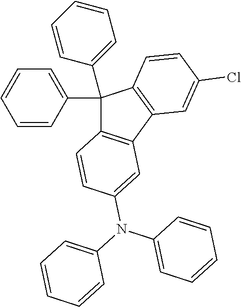 [SMILES: Clc1ccc2c(c1)-c1cc(N(c3ccccc3)c3ccccc3)ccc1C2(c1ccccc1)c1ccccc1] | Sub 3-150 |
| 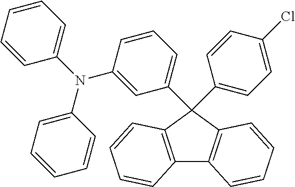 [SMILES: Clc1ccc(C2(c3cccc(N(c4ccccc4)c4ccccc4)c3)c3ccccc3-c3ccccc32)cc1] | Sub 3-151 |
| 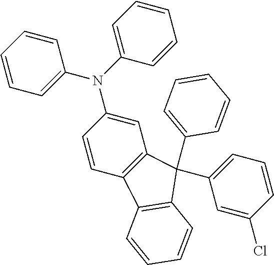 [SMILES: Clc1cccc(C2(c3ccccc3)c3ccccc3-c3ccc(N(c4ccccc4)c4ccccc4)cc32)c1] | Sub 3-152 |
| 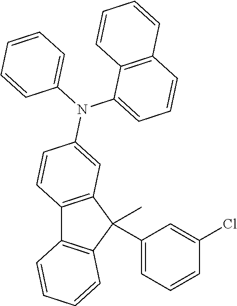 [SMILES: CC1(c2cccc(Cl)c2)c2ccccc2-c2ccc(N(c3ccccc3)c3cccc4ccccc34)cc21] | Sub 3-153 |
| 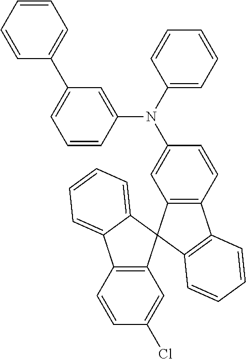 [SMILES: Clc1ccc2c(c1)C1(c3ccccc3-2)c2ccccc2-c2ccc(N(c3ccccc3)c3cccc(-c4ccccc4)c3)cc21] | Sub 3-154 |
| 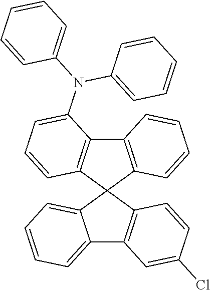 [SMILES: Clc1ccc2c(c1)-c1ccccc1C21c2ccccc2-c2c(N(c3ccccc3)c3ccccc3)cccc21] | Sub 3-155 |
| 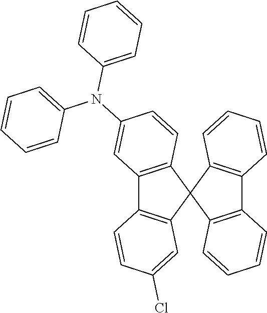 [SMILES: Clc1ccc2c(c1)C1(c3ccccc3-c3ccccc31)c1ccc(N(c3ccccc3)c3ccccc3)cc1-2] | Sub 3-156 |
| 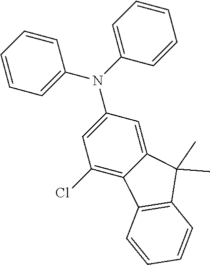 [SMILES: CC1(C)c2ccccc2-c2c(Cl)cc(N(c3ccccc3)c3ccccc3)cc21] | Sub 3-157 |
| 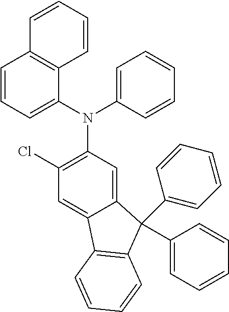 [SMILES: Clc1cc2c(cc1N(c1ccccc1)c1cccc3ccccc13)C(c1ccccc1)(c1ccccc1)c1ccccc1-2] | Sub 3-158 |
| 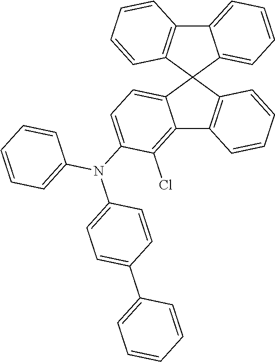 [SMILES: Clc1c(N(c2ccccc2)c2ccc(-c3ccccc3)cc2)ccc2c1-c1ccccc1C21c2ccccc2-c2ccccc21] | Sub 3-159 |
| 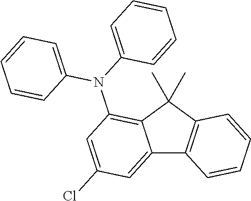 [SMILES: CC1(C)c2ccccc2-c2cc(Cl)cc(N(c3ccccc3)c3ccccc3)c21] | Sub 3-160 |
| 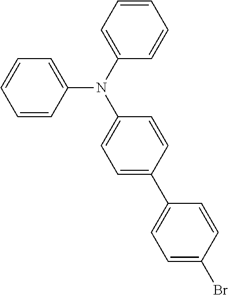 [SMILES: Brc1ccc(-c2ccc(N(c3ccccc3)c3ccccc3)cc2)cc1] | Sub 3-161 |
| 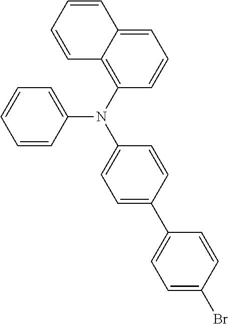 [SMILES: Brc1ccc(-c2ccc(N(c3ccccc3)c3cccc4ccccc34)cc2)cc1] | Sub 3-162 |
| 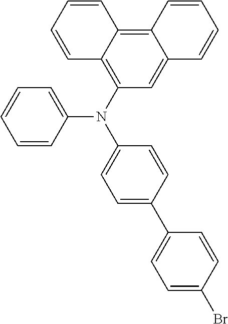 [SMILES: Brc1ccc(-c2ccc(N(c3ccccc3)c3cc4ccccc4c4ccccc34)cc2)cc1] | Sub 3-163 |
| 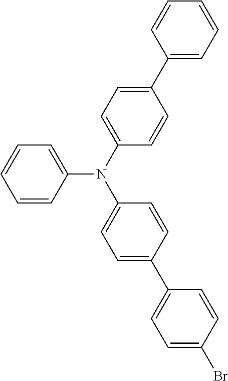 [SMILES: Brc1ccc(-c2ccc(N(c3ccccc3)c3ccc(-c4ccccc4)cc3)cc2)cc1] | Sub 3-164 |
| 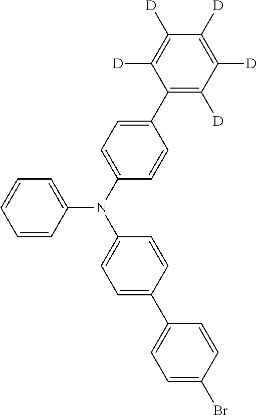 [SMILES: [2H]c1c([2H])c([2H])c(-c2ccc(N(c3ccccc3)c3ccc(-c4ccc(Br)cc4)cc3)cc2)c([2H])c1[2H]] | Sub 3-165 |
| 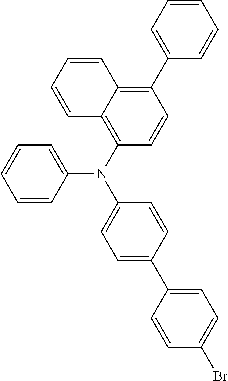 [SMILES: Brc1ccc(-c2ccc(N(c3ccccc3)c3ccc(-c4ccccc4)c4ccccc34)cc2)cc1] | Sub 3-166 |
| 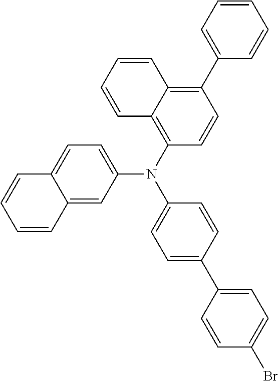 [SMILES: Brc1ccc(-c2ccc(N(c3ccc4ccccc4c3)c3ccc(-c4ccccc4)c4ccccc34)cc2)cc1] | Sub 3-167 |
| 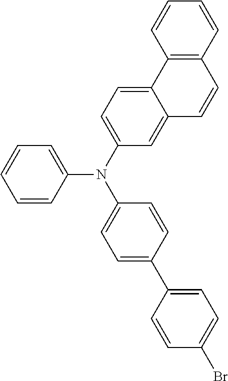 [SMILES: Brc1ccc(-c2ccc(N(c3ccccc3)c3ccc4c(ccc5ccccc54)c3)cc2)cc1] | Sub 3-168 |
| 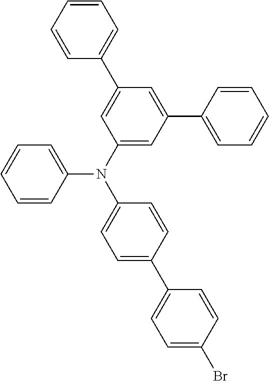 [SMILES: Brc1ccc(-c2ccc(N(c3ccccc3)c3cc(-c4ccccc4)cc(-c4ccccc4)c3)cc2)cc1] | Sub 3-169 |
| 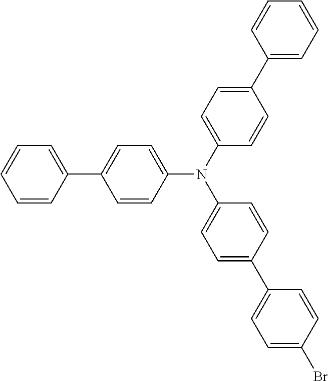 [SMILES: Brc1ccc(-c2ccc(N(c3ccc(-c4ccccc4)cc3)c3ccc(-c4ccccc4)cc3)cc2)cc1] | Sub 3-170 |
| 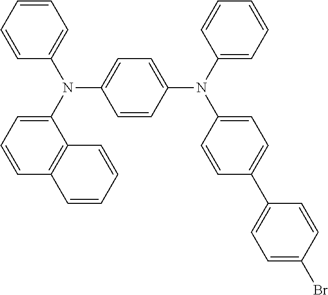 [SMILES: Brc1ccc(-c2ccc(N(c3ccccc3)c3ccc(N(c4ccccc4)c4cccc5ccccc45)cc3)cc2)cc1] | Sub 3-171 |
| 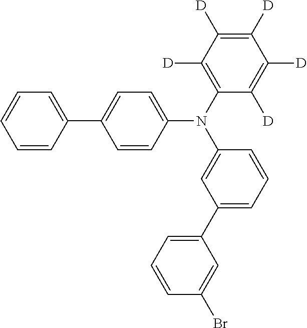 [SMILES: [2H]c1c([2H])c([2H])c(N(c2ccc(-c3ccccc3)cc2)c2cccc(-c3cccc(Br)c3)c2)c([2H])c1[2H]] | Sub 3-172 |
| 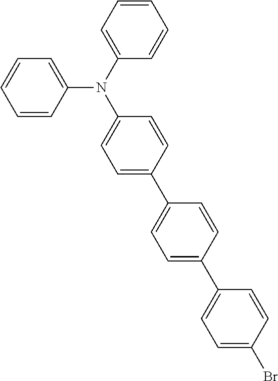 [SMILES: Brc1ccc(-c2ccc(-c3ccc(N(c4ccccc4)c4ccccc4)cc3)cc2)cc1] | Sub 3-173 |
| 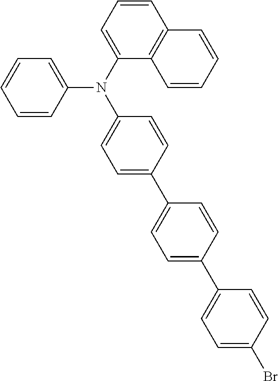 [SMILES: Brc1ccc(-c2ccc(-c3ccc(N(c4ccccc4)c4cccc5ccccc45)cc3)cc2)cc1] | Sub 3-174 |
| 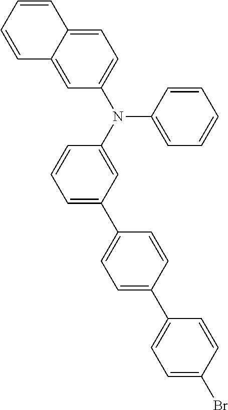 [SMILES: Brc1ccc(-c2ccc(-c3cccc(N(c4ccccc4)c4ccc5ccccc5c4)c3)cc2)cc1] | Sub 3-175 |
| 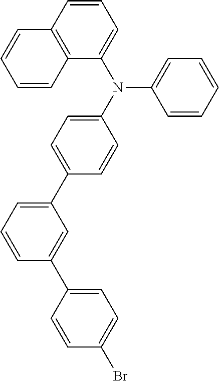 [SMILES: Brc1ccc(-c2cccc(-c3ccc(N(c4ccccc4)c4cccc5ccccc45)cc3)c2)cc1] | Sub 3-176 |
| 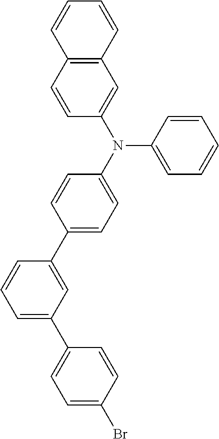 [SMILES: Brc1ccc(-c2cccc(-c3ccc(N(c4ccccc4)c4ccc5ccccc5c4)cc3)c2)cc1] | Sub 3-177 |
| 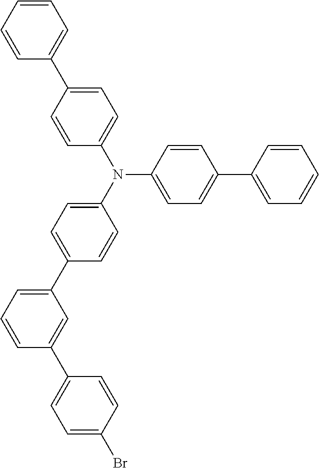 [SMILES: Brc1ccc(-c2cccc(-c3ccc(N(c4ccc(-c5ccccc5)cc4)c4ccc(-c5ccccc5)cc4)cc3)c2)cc1] | Sub 3-178 |
| 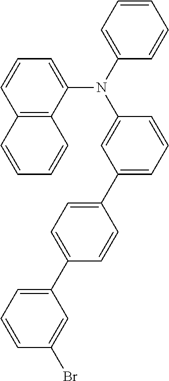 [SMILES: Brc1cccc(-c2ccc(-c3cccc(N(c4ccccc4)c4cccc5ccccc45)c3)cc2)c1] | Sub 3-179 |
| 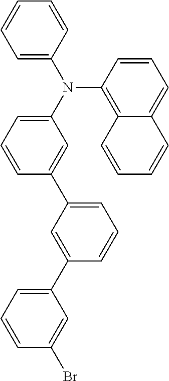 [SMILES: Brc1cccc(-c2cccc(-c3cccc(N(c4ccccc4)c4cccc5ccccc45)c3)c2)c1] | Sub 3-180 |
| 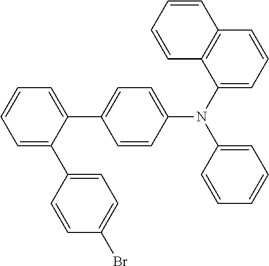 [SMILES: Brc1ccc(-c2ccccc2-c2ccc(N(c3ccccc3)c3cccc4ccccc34)cc2)cc1] | Sub 3-181 |
| 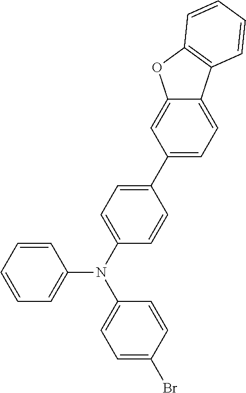 [SMILES: Brc1ccc(N(c2ccccc2)c2ccc(-c3ccc4c(c3)oc3ccccc34)cc2)cc1] | Sub 3-182 |
| 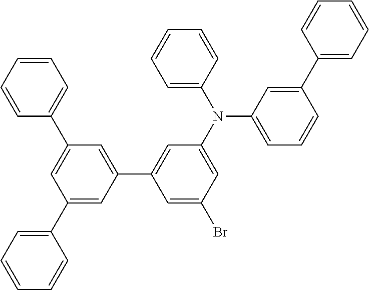 [SMILES: Brc1cc(-c2cc(-c3ccccc3)cc(-c3ccccc3)c2)cc(N(c2ccccc2)c2cccc(-c3ccccc3)c2)c1] | Sub 3-183 |
| 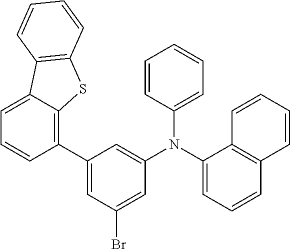 [SMILES: Brc1cc(-c2cccc3c2sc2ccccc23)cc(N(c2ccccc2)c2cccc3ccccc23)c1] | Sub 3-184 |
| 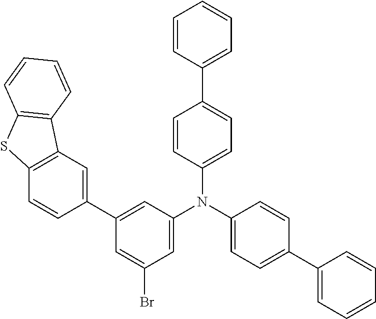 [SMILES: Brc1cc(-c2ccc3sc4ccccc4c3c2)cc(N(c2ccc(-c3ccccc3)cc2)c2ccc(-c3ccccc3)cc2)c1] | Sub 3-185 |
| 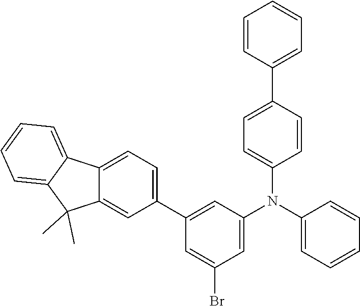 [SMILES: CC1(C)c2ccccc2-c2ccc(-c3cc(Br)cc(N(c4ccccc4)c4ccc(-c5ccccc5)cc4)c3)cc21] | Sub 3-186 |
| 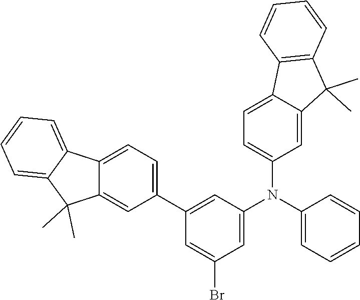 [SMILES: CC1(C)c2ccccc2-c2ccc(-c3cc(Br)cc(N(c4ccccc4)c4ccc5c(c4)C(C)(C)c4ccccc4-5)c3)cc21] | Sub 3-187 |
| 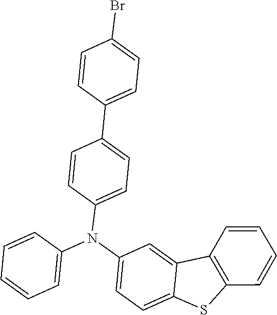 [SMILES: Brc1ccc(-c2ccc(N(c3ccccc3)c3ccc4sc5ccccc5c4c3)cc2)cc1] | Sub 3-188 |
| 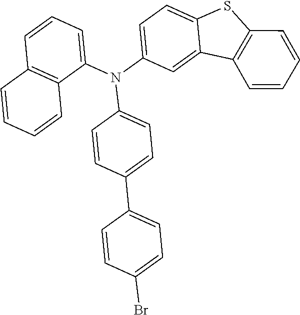 [SMILES: Brc1ccc(-c2ccc(N(c3ccc4sc5ccccc5c4c3)c3cccc4ccccc34)cc2)cc1] | Sub 3-189 |
| 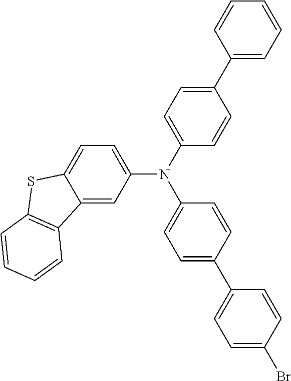 [SMILES: Brc1ccc(-c2ccc(N(c3ccc(-c4ccccc4)cc3)c3ccc4sc5ccccc5c4c3)cc2)cc1] | Sub 3-190 |
| 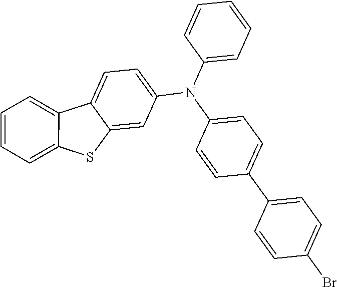 [SMILES: Brc1ccc(-c2ccc(N(c3ccccc3)c3ccc4c(c3)sc3ccccc34)cc2)cc1] | Sub 3-191 |
| 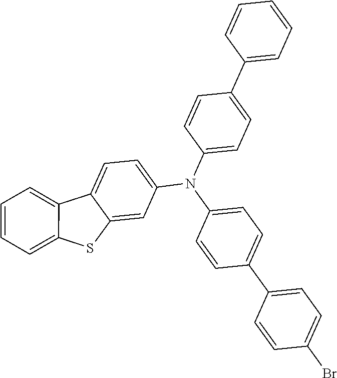 [SMILES: Brc1ccc(-c2ccc(N(c3ccc(-c4ccccc4)cc3)c3ccc4c(c3)sc3ccccc34)cc2)cc1] | Sub 3-192 |
| 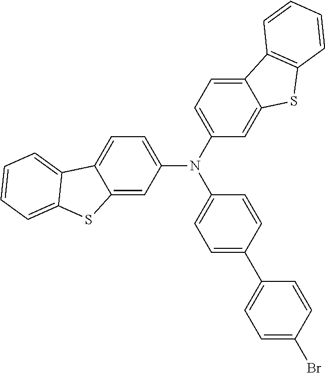 [SMILES: Brc1ccc(-c2ccc(N(c3ccc4c(c3)sc3ccccc34)c3ccc4c(c3)sc3ccccc34)cc2)cc1] | Sub 3-193 |
| 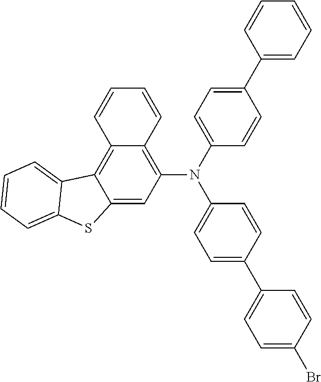 [SMILES: Brc1ccc(-c2ccc(N(c3ccc(-c4ccccc4)cc3)c3cc4sc5ccccc5c4c4ccccc34)cc2)cc1] | Sub 3-194 |
| 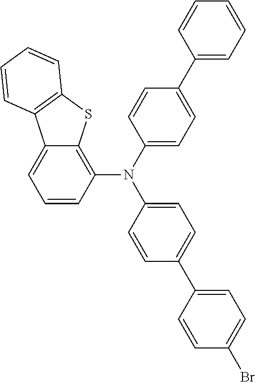 [SMILES: Brc1ccc(-c2ccc(N(c3ccc(-c4ccccc4)cc3)c3cccc4c3sc3ccccc34)cc2)cc1] | Sub 3-195 |
| 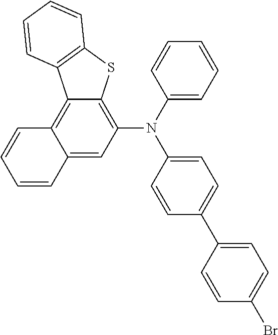 [SMILES: Brc1ccc(-c2ccc(N(c3ccccc3)c3cc4ccccc4c4c3sc3ccccc34)cc2)cc1] | Sub 3-196 |
| 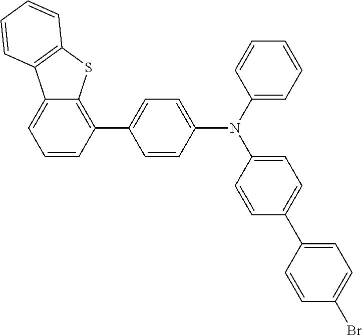 [SMILES: Brc1ccc(-c2ccc(N(c3ccccc3)c3ccc(-c4cccc5c4sc4ccccc45)cc3)cc2)cc1] | Sub 3-197 |
| 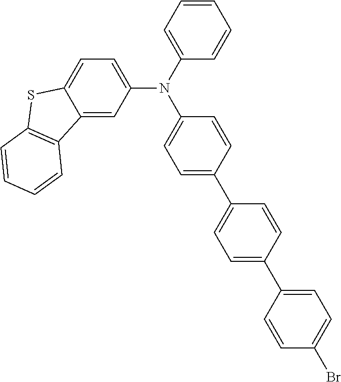 [SMILES: Brc1ccc(-c2ccc(-c3ccc(N(c4ccccc4)c4ccc5sc6ccccc6c5c4)cc3)cc2)cc1] | Sub 3-198 |
| 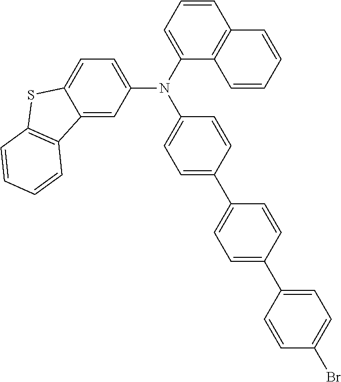 [SMILES: Brc1ccc(-c2ccc(-c3ccc(N(c4ccc5sc6ccccc6c5c4)c4cccc5ccccc45)cc3)cc2)cc1] | Sub 3-199 |
| 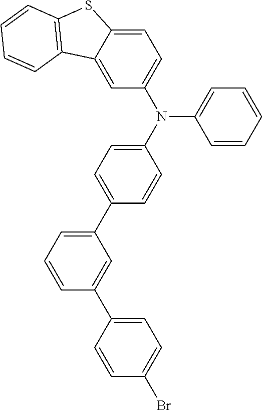 [SMILES: Brc1ccc(-c2cccc(-c3ccc(N(c4ccccc4)c4ccc5sc6ccccc6c5c4)cc3)c2)cc1] | Sub 3-200 |
| 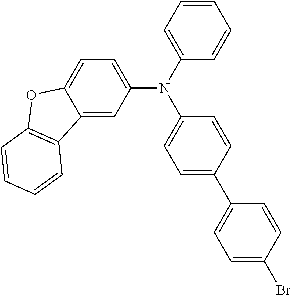 [SMILES: Brc1ccc(-c2ccc(N(c3ccccc3)c3ccc4oc5ccccc5c4c3)cc2)cc1] | Sub 3-201 |
| 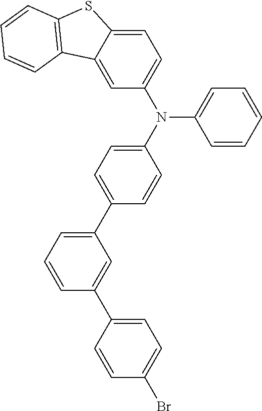 [SMILES: Brc1ccc(-c2cccc(-c3ccc(N(c4ccccc4)c4ccc5sc6ccccc6c5c4)cc3)c2)cc1] | Sub 3-202 |
| 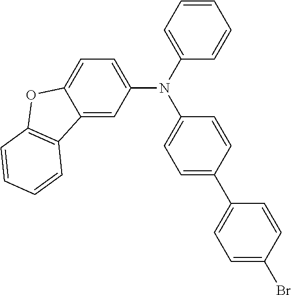 [SMILES: Brc1ccc(-c2ccc(N(c3ccccc3)c3ccc4oc5ccccc5c4c3)cc2)cc1] | Sub 3-203 |
| 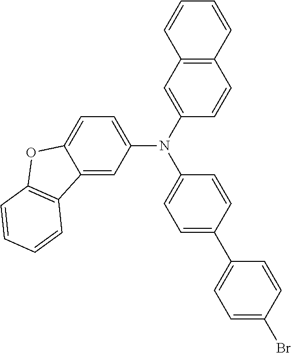 [SMILES: Brc1ccc(-c2ccc(N(c3ccc4ccccc4c3)c3ccc4oc5ccccc5c4c3)cc2)cc1] | Sub 3-204 |
| 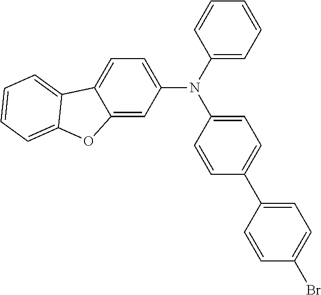 [SMILES: Brc1ccc(-c2ccc(N(c3ccccc3)c3ccc4c(c3)oc3ccccc34)cc2)cc1] | Sub 3-205 |
| 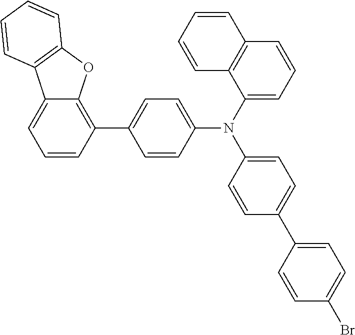 [SMILES: Brc1ccc(-c2ccc(N(c3ccc(-c4cccc5c4oc4ccccc45)cc3)c3cccc4ccccc34)cc2)cc1] | Sub 3-206 |
| 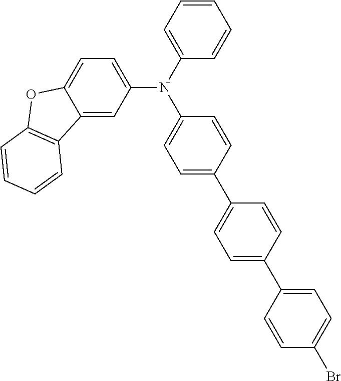 [SMILES: Brc1ccc(-c2ccc(-c3ccc(N(c4ccccc4)c4ccc5oc6ccccc6c5c4)cc3)cc2)cc1] | Sub 3-207 |
| 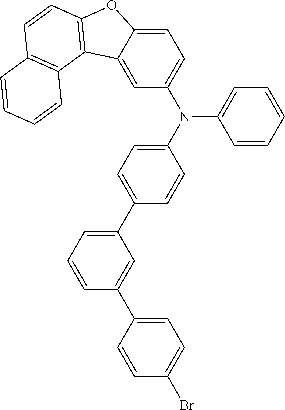 [SMILES: Brc1ccc(-c2cccc(-c3ccc(N(c4ccccc4)c4ccc5oc6ccc7ccccc7c6c5c4)cc3)c2)cc1] | Sub 3-208 |
| 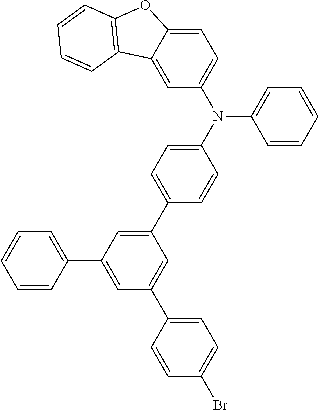 [SMILES: Brc1ccc(-c2cc(-c3ccccc3)cc(-c3ccc(N(c4ccccc4)c4ccc5oc6ccccc6c5c4)cc3)c2)cc1] | Sub 3-209 |
| 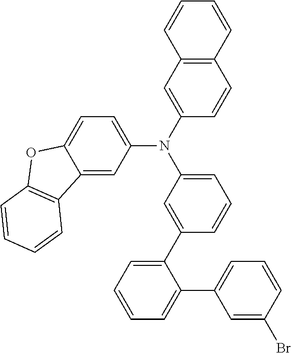 [SMILES: Brc1cccc(-c2ccccc2-c2cccc(N(c3ccc4ccccc4c3)c3ccc4oc5ccccc5c4c3)c2)c1] | Sub 3-210 |
| 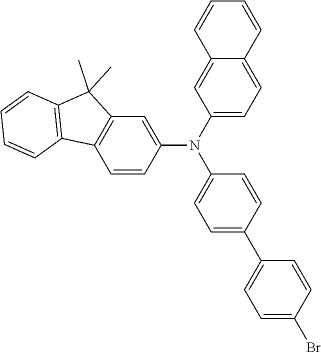 [SMILES: CC1(C)c2ccccc2-c2ccc(N(c3ccc(-c4ccc(Br)cc4)cc3)c3ccc4ccccc4c3)cc21] | Sub 3-211 |
| 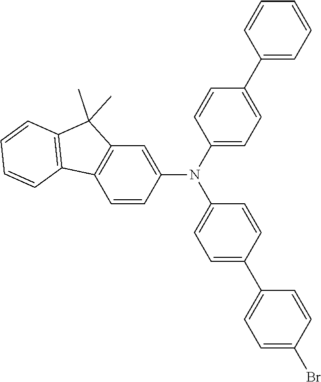 [SMILES: CC1(C)c2ccccc2-c2ccc(N(c3ccc(-c4ccccc4)cc3)c3ccc(-c4ccc(Br)cc4)cc3)cc21] | Sub 3-212 |
| 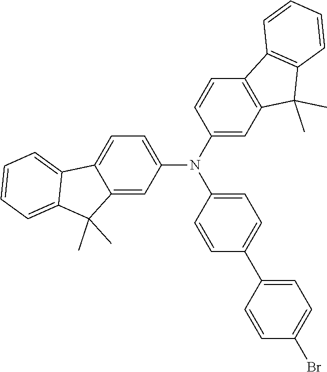 [SMILES: CC1(C)c2ccccc2-c2ccc(N(c3ccc(-c4ccc(Br)cc4)cc3)c3ccc4c(c3)C(C)(C)c3ccccc3-4)cc21] | Sub 3-213 |
| 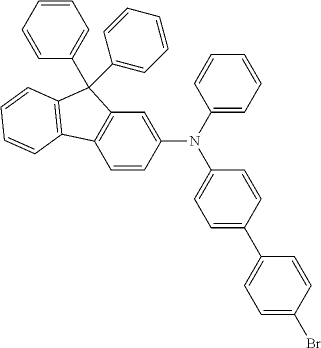 [SMILES: Brc1ccc(-c2ccc(N(c3ccccc3)c3ccc4c(c3)C(c3ccccc3)(c3ccccc3)c3ccccc3-4)cc2)cc1] | Sub 3-214 |
| 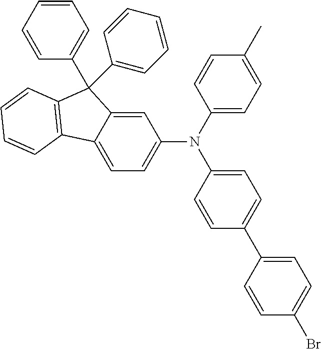 [SMILES: Cc1ccc(N(c2ccc(-c3ccc(Br)cc3)cc2)c2ccc3c(c2)C(c2ccccc2)(c2ccccc2)c2ccccc2-3)cc1] | Sub 3-215 |
| 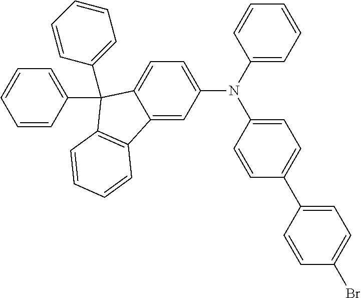 [SMILES: Brc1ccc(-c2ccc(N(c3ccccc3)c3ccc4c(c3)-c3ccccc3C4(c3ccccc3)c3ccccc3)cc2)cc1] | Sub 3-216 |
| 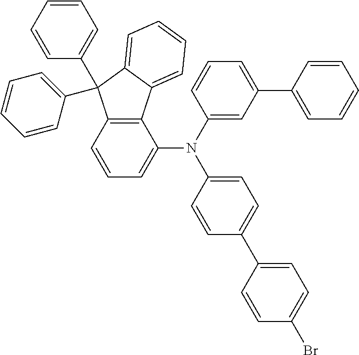 [SMILES: Brc1ccc(-c2ccc(N(c3cccc(-c4ccccc4)c3)c3cccc4c3-c3ccccc3C4(c3ccccc3)c3ccccc3)cc2)cc1] | Sub 3-217 |
| 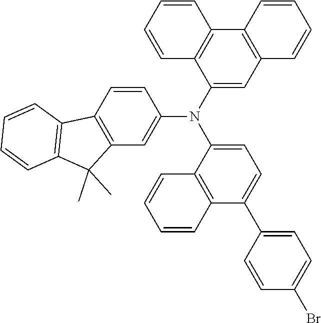 [SMILES: CC1(C)c2ccccc2-c2ccc(N(c3ccc(-c4ccc(Br)cc4)c4ccccc34)c3cc4ccccc4c4ccccc34)cc21] | Sub 3-218 |
| 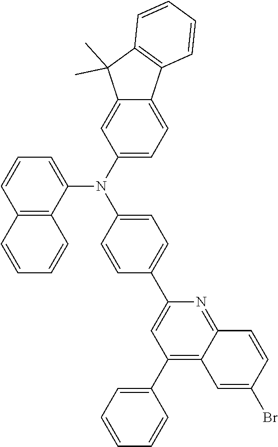 [SMILES: CC1(C)c2ccccc2-c2ccc(N(c3ccc(-c4cc(-c5ccccc5)c5cc(Br)ccc5n4)cc3)c3cccc4ccccc34)cc21] | Sub 3-219 |
| 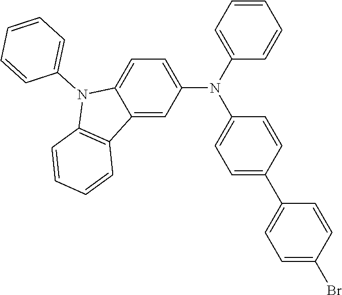 [SMILES: Brc1ccc(-c2ccc(N(c3ccccc3)c3ccc4c(c3)c3ccccc3n4-c3ccccc3)cc2)cc1] | Sub 3-220 |
| 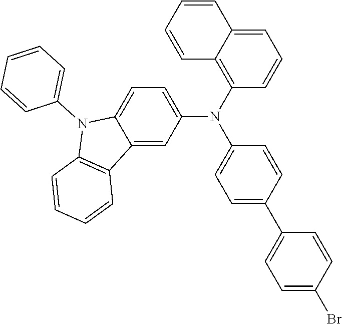 [SMILES: Brc1ccc(-c2ccc(N(c3ccc4c(c3)c3ccccc3n4-c3ccccc3)c3cccc4ccccc34)cc2)cc1] | Sub 3-221 |
| 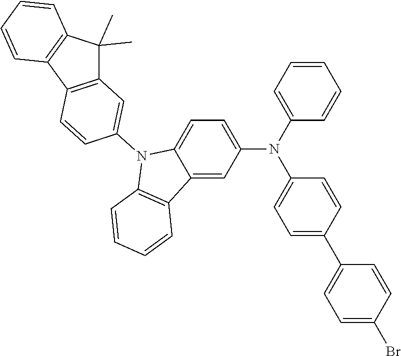 [SMILES: CC1(C)c2ccccc2-c2ccc(-n3c4ccccc4c4cc(N(c5ccccc5)c5ccc(-c6ccc(Br)cc6)cc5)ccc43)cc21] | Sub 3-222 |
| 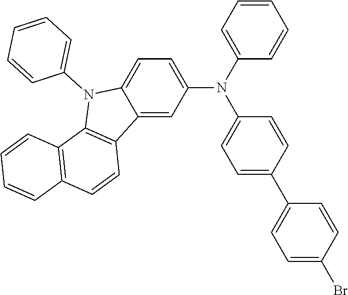 [SMILES: Brc1ccc(-c2ccc(N(c3ccccc3)c3ccc4c(c3)c3ccc5ccccc5c3n4-c3ccccc3)cc2)cc1] | Sub 3-223 |
| 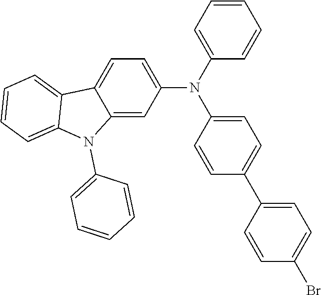 [SMILES: Brc1ccc(-c2ccc(N(c3ccccc3)c3ccc4c5ccccc5n(-c5ccccc5)c4c3)cc2)cc1] | Sub 3-224 |
| 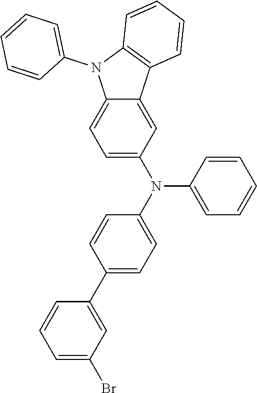 [SMILES: Brc1cccc(-c2ccc(N(c3ccccc3)c3ccc4c(c3)c3ccccc3n4-c3ccccc3)cc2)c1] | Sub 3-225 |
| 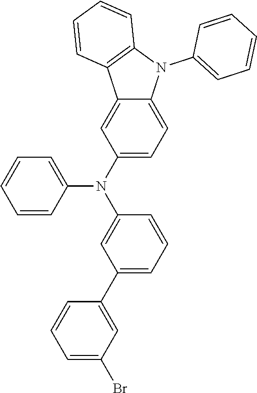 [SMILES: Brc1cccc(-c2cccc(N(c3ccccc3)c3ccc4c(c3)c3ccccc3n4-c3ccccc3)c2)c1] | Sub 3-226 |

| Compound | FD-MS |
|---|---|
| Sub 3-1 | m/z = 429.02 (C₂₄H₁₆BrNS = 430.36) |
| Sub 3-2 | m/z = 413.04 (C₂₄H₁₆BrNO = 414.30) |
| Sub 3-3 | m/z = 463.06 (C₂₈H₁₅BrNO = 454.36) |
| Sub 3-4 | m/z = 385.07 (C₂₄H₁₆ClNS = 385.91) |
| Sub 3-5 | m/z = 435.08 (C₂₈H₁₈ClNS = 435.97) |
| Sub 3-6 | m/z = 567.09 (C₃₆H₂₂ClNS₂ = 568.15) |
| Sub 3-7 | m/z = 551.11 (C₃₆H₂₂ClNOS = 552.09) |
| Sub 3-8 | m/z = 501.13 (C₃₃H₂₄ClNS = 502.07) |
| Sub 3-9 | m/z = 445.12 (C₃₀H₂₀ClNO = 445.95) |
| Sub 3-10 | m/z = 445.12 (C₃₀H₂₀ClNO = 445.95) |
| Sub 3-11 | m/z = 385.07 (C₂₄H₁₈ClNS = 385.91) |
| Sub 3-12 | m/z = 491.06 (C₃₈H₁₈ClNS₂ = 495.05) |
| Sub 3-13 | m/z = 567.09 (C₃₆H₂₂ClNS₂ = 568.15) |
| Sub 3-14 | m/z = 541.07 (C₃₄H₂₀ClNS₂ = 542.11) |
| Sub 3-15 | m/z = 575.11 (C₃₈H₂₂ClNOS = 576.11) |
| Sub 3-16 | m/z = 552.11 (C₃₅H₂₁ClN₂OS = 553.08) |
| Sub 3-17 | m/z = 626.16 (C₄₂H₂₇ClN₂S = 627.20) |
| Sub 3-18 | m/z = 643.12 (C₄₂H₂₆ClNS₂ = 644.25) |
| Sub 3-19 | m/z = 435.08 (C₂₈H₁₈ClNS = 435.97) |
| Sub 3-20 | m/z = 369.09 (C₂₄H₁₆ClNO = 369.85) |
| Sub 3-21 | m/z = 475.08 (C₃₀H₁₈ClNOS = 475.99) |
| Sub 3-22 | m/z = 565.09 (C₃₆H₂₀ClNO₂S = 566.07) |
| Sub 3-23 | m/z = 519.14 (C₃₆H₂₂ClNO = 520.03) |
| Sub 3-24 | m/z = 385.07 (C₂₄H₁₆ClNS = 385.91) |
| Sub 3-25 | m/z = 435.08 (C₂₈H₁₈ClNS = 435.97) |
| Sub 3-26 | m/z = 567.09 (C₃₆H₂₂ClNS₂ = 568.15) |
| Sub 3-27 | m/z = 597.04 (C₃₆H₂₀ClNS₃ = 598.19) |
| Sub 3-28 | m/z = 435.08 (C₂₈H₁₈ClNS = 435.97) |
| Sub 3-29 | m/z = 435.08 (C₂₈H₁₈ClNS = 435.97) |
| Sub 3-30 | m/z = 485.10 (C₃₂H₂₀ClNS = 486.03) |
| Sub 3-31 | m/z = 591.14 (C₃₉H₂₆ClNOS = 592.15) |
| Sub 3-32 | m/z = 559.13 (C₃₈H₂₂ClNO₂ = 560.05) |
| Sub 3-33 | m/z = 677.16 (C₄₆H₂₈ClNOS = 678.25) |
| Sub 3-34 | m/z = 385.07 (C₂₄H₁₆ClNOS = 385.91) |
| Sub 3-35 | m/z = 399.08 (C₂₅H₁₈ClNS = 399.94) |
| Sub 3-36 | m/z = 435.08 (C₂₈H₁₈ClNS = 435.97) |
| Sub 3-37 | m/z = 461.10 (C₃₀H₂₀ClNS = 462.01) |
| Sub 3-38 | m/z = 511.12 (C₃₄H₂₂ClNS = 512.07) |
| Sub 3-39 | m/z = 399.08 (C₂₅H₁₈ClNS = 399.94) |
| Sub 3-40 | m/z = 461.10 (C₃₀H₂₀ClNS = 462.01) |
| Sub 3-41 | m/z = 491.06 (C₃₀H₁₈ClNS₂ = 492.05) |
| Sub 3-42 | m/z = 491.06 (C₃₀H₁₈ClNS₂ = 492.05) |
| Sub 3-43 | m/z = 491.06 (C₃₀H₁₈ClNS₂ = 492.05) |
| Sub 3-44 | m/z = 541.07 (C₃₄H₂₀ClNS₂ = 542.11) |
| Sub 3-45 | m/z = 643.12 (C₄₂H₂₆ClNS₂ = 644.25) |
| Sub 3-46 | m/z = 607.12 (C₃₉H₂₆ClNS₂ = 608.21) |
| Sub 3-47 | m/z = 541.07 (C₃₄H₂₀ClNS₂ = 542.11) |
| Sub 3-48 | m/z = 475.08 (C₃₀H₁₈ClNOS = 475.99) |
| Sub 3-49 | m/z = 626.16 (C₄₂H₂₇ClN₂S = 627.20) |
| Sub 3-50 | m/z = 435.08 (C₂₈H₁₈ClNS = 435.97) |
| Sub 3-51 | m/z = 435.08 (C₂₈H₁₈ClNS = 435.97) |
| Sub 3-52 | m/z = 435.08 (C₂₈H₁₈ClNS = 435.97) |
| Sub 3-53 | m/z = 541.07 (C₃₄H₂₀ClNS₂ = 542.11) |
| Sub 3-54 | m/z = 555.09 (C₃₅H₂₂ClNS₂ = 556.14) |
| Sub 3-55 | m/z = 525.10 (C₃₄H₂₀ClNOS = 526.05) |
| Sub 3-56 | m/z = 513.13 (C₃₄H₂₄ClNS = 514.08) |
| Sub 3-57 | m/z = 485.10 (C₃₂H₂₀ClNS = 486.03) |
| Sub 3-58 | m/z = 537.13 (C₃₆H₂₄ClNS = 538.11) |
| Sub 3-59 | m/z = 626.16 (C₄₂H₂₇ClN₂S = 627.20) |
| Sub 3-60 | m/z = 369.09 (C₂₄H₁₆ClNO = 369.85) |
| Sub 3-61 | m/z = 445.12 (C₃₀H₂₀ClNO = 445.95) |
| Sub 3-62 | m/z = 495.14 (C₃₄H₂₂ClNO = 496.01) |
| Sub 3-63 | m/z = 459.10 (C₃₀H₁₈ClNO₂ = 459.93) |
| Sub 3-64 | m/z = 534.15 (C₃₆H₂₃ClN₂O = 535.04) |
| Sub 3-65 | m/z = 535.17 (C₃₇H₂₆ClNO = 536.07) |
| Sub 3-66 | m/z = 525.10 (C₃₄H₂₀ClNOS = 526.05) |
| Sub 3-67 | m/z = 385.07 (C₂₄H₁₆ClNS = 385.91) |
| Sub 3-68 | m/z = 435.08 (C₂₈H₁₈ClNS = 435.97) |
| Sub 3-69 | m/z = 461.10 (C₃₀H₂₀ClNS = 462.01) |
| Sub 3-70 | m/z = 611.15 (C₄₂H₂₆ClNS = 612.19) |
| Sub 3-71 | m/z = 567.09 (C₁₄H₂₂ClNS₂ = 568.15) |
| Sub 3-72 | m/z = 541.07 (C₃₄H₂₀ClNS₂ = 542.11) |
| Sub 3-73 | m/z = 475.08 (C₃₀H₁₆ClNOS = 475.99) |
| Sub 3-74 | m/z = 551.11 (C₃₆H₂₇ClNOS = 552.09) |
| Sub 3-75 | m/z = 591.14 (C₃₉H₂₈ClNOS = 592.15) |
| Sub 3-76 | m/z = 435.08 (C₂₈H₁₈ClNS = 435.97) |
| Sub 3-77 | m/z = 591.09 (C₃₈H₂₂ClNS₂ = 592.17) |
| Sub 3-78 | m/z = 537.13 (C₃₆H₂₄ClNS = 538.11) |
| Sub 3-79 | m/z = 379.15 (C₂₄H₆D₁₀ClNO = 379.91) |
| Sub 3-80 | m/z = 459.10 (C₃₀H₁₆ClNO₂ = 459.93) |
| Sub 3-81 | m/z = 445.12 (C₃₀H₂₀ClNO = 445.95) |
| Sub 3-82 | m/z = 429.02 (C₂₄H₁₆BrNS = 430.36) |
| Sub 3-83 | m/z = 443.03 (C₂₅H₁₆BrNS = 444.39) |
| Sub 3-84 | m/z = 479.03 (C₂₈H₁₆BrNS = 480.42) |
| Sub 3-85 | m/z = 485.10 (C₃₂H₂₀ClNS = 486.03) |
| Sub 3-86 | m/z = 545.12 (C₃₄H₂₄ClNO₂S = 546.08) |
| Sub 3-87 | m/z = 413.04 (C₂₄H₁₆BrNO = 414.30) |
| Sub 3-88 | m/z = 529.10 (C₃₃H₂₄BrNO = 530.47) |
| Sub 3-89 | m/z = 589.10 (C₃₈H₂₄BrNO = 590.52) |
| Sub 3-90 | m/z = 385.07 (C₂₄H₁₆ClNS = 385.91) |
| Sub 3-91 | m/z = 537.13 (C₃₆H₂₄ClNS = 538.11) |
| Sub 3-92 | m/z = 491.06 (C₃₀H₁₈ClNS₂ = 492.05) |
| Sub 3-93 | m/z = 567.09 (C₃₆H₂₂ClNS₂ = 568.15) |
| Sub 3-94 | m/z = 567.09 (C₃₅H₂₂ClNS₂ = 568.15) |
| Sub 3-95 | m/z = 551.11 (C₃₆H₂₂ClNOS = 552.09) |
| Sub 3-96 | m/z = 683.15 (C₄₅H₃₀ClNS₂ = 684.31) |
| Sub 3-97 | m/z = 435.08 (C₂₈H₁₈ClNS = 435.97) |
| Sub 3-98 | m/z = 591.09 (C₃₈H₂₂ClNS₂ = 592.17) |
| Sub 3-99 | m/z = 748.18 (C₄₉H₃₃ClN₂S₂ = 749.39) |
| Sub 3-100 | m/z = 369.09 (C₂₄H₁₈ClNO = 369.85) |
| Sub 3-101 | m/z = 419.11 (C₂₈H₁₈ClNO = 419.91) |
| Sub 3-102 | m/z = 475.08 (C₃₀H₁₈ClNOS = 475.99) |
| Sub 3-103 | m/z = 615.13 (C₄₂H₂₄ClNOS = 626.17) |
| Sub 3-104 | m/z = 429.02 (C₂₄H₁₆BrNS = 430.36) |
| Sub 3-105 | m/z = 479.03 (C₂₈H₁₈BrNS = 480.42) |
| Sub 3-106 | m/z = 505.05 (C₃₀H₂₀BrNS = 506.46) |
| Sub 3-107 | m/z = 435.08 (C₂₈H₁₈ClNS = 435.97) |
| Sub 3-108 | m/z = 541.07 (C₃₄H₂₀ClNS₂ = 542.11) |
| Sub 3-109 | m/z = 489.07 (C₃₀H₂₀BrNO = 490.40) |
| Sub 3-110 | m/z = 565.10 (C₃₆H₂₄BrNO = 566.50) |
| Sub 3-111 | m/z = 716.15 (C₆₇H₂₉BrN₂O = 717.67) |
| Sub 3-112 | m/z = 385.07 (C₂₄H₁₆ClNS = 385.91) |
| Sub 3-113 | m/z = 511.12 (C₃₄H₂₂ClNS = 512.07) |
| Sub 3-114 | m/z = 557.14 (C₃₅H₂₈ClNSS [symbol] = 558.21) |
| Sub 3-115 | m/z = 495.14 (C₃₄H₂₂ClNO = 496.01) |
| Sub 3-116 | m/z = 575.11 (C₃₈H₂₂ClNOS = 576.11) |
| Sub 3-117 | m/z = 469.12 (C₃₂H₂₀ClNO = 469.97) |
| Sub 3-118 | m/z = 469.12 (C₃₂H₂₀ClNO = 469.97) |
| Sub 3-119 | m/z = 635.17 (C₄₄H₂₆ClNO₂ = 636.15) |
| Sub 3-120 | m/z = 650.16 (C₄₄H₂₇ClN₂S = 651.22) |
| Sub 3-121 | m/z = 585.13 (C₄₀H₂₄ClNS = 586.15) |
| Sub 3-122 | m/z = 673.08 (C₄₂H₂₄ClNS₃ = 674.29) |
| Sub 3-123 | m/z = 491.06 (C₃₆H₃₂ClNS₂ = 492.05) |
| Sub 3-124 | m/z = 461.1 (C₃₀H₂₈ClNS = 462.01) |
| Sub 3-125 | m/z = 641.16 (C [symbol] H₂₈ClNOS = 642.21) |
| Sub 3-126 | m/z = 445.16 (C₃₁H₂₄ClN = 445.99) |
| Sub 3-127 | m/z = 471.18 (C₃₃H₂₆ClN = 472.03) |
| Sub 3-128 | m/z = 395.15 (C₂₇H₂₂ClN = 395.93) |
| Sub 3-129 | m/z = 471.18 (C₃₃H₂₆ClN = 472.03) |
| Sub 3-130 | m/z = 395.14 (C₂₇H₂₂ClN = 395.93) |
| Sub 3-131 | m/z = 511.21 (C₃₆H₂₀ClN = 512.09) |
| Sub 3-132 | m/z = 423.18 (C₂₉H₂₆ClN = 423.98) |
| Sub 3-133 | m/z = 569.19 (C₄₁H₂₈ClN = 570.13) |
| Sub 3-134 | m/z = 595.12 (C₄₃H₃₀ClN = 596.17) |
| Sub 3-135 | m/z = 519.18 (C₃₇H₂₆ClN = 520.07) |
| Sub 3-136 | m/z = 567.18 (C₄₁H₂₆ClN = 568.12) |
| Sub 3-137 | m/z = 515.14 (C₃₇H₂₂ClN = 516.04) |
| Sub 3-138 | m/z = 517.16 (C₃₇H₂₄ClN = 518.06) |
| Sub 3-139 | m/z = 607.17 (C₄₃H₂₆ClNO = 608.14) |
| Sub 3-140 | m/z = 517.16 (C₃₇H₂₄ClN = 518.06) |
| Sub 3-141 | m/z = 607.17 (C₄₃H₂₆ClNO = 608.14) |
| Sub 3-142 | m/z = 395.14 (C₂₇H₂₂ClN = 395.93) |
| Sub 3-143 | m/z = 485.15 (C₃₃H₂₄ClNO = 486.01) |
| Sub 3-148 | m/z = 570.18 (C₃₆H₂₉ClN = 508.06) |
| Sub 3-149 | m/z = 457.16 (C₃₂H₂₄ClN = 458) |
| Sub 3-150 | m/z = 519.18 (C₃₇H₂₆ClN = 520.07) |
| Sub 3-161 | m/z = 399.06 (C₂₄H₁₈BrN = 400.32) |
| Sub 3-162 | m/z = 449.08 (C₂₈H₂₀BrN = 450.38) |
| Sub 3-163 | m/z = 499.09 (C₃₂H₂₂BrN = 500.44) |
| Sub 3-164 | m/z = 475.09 (C₃₀H₂₂BrN = 476.42) |
| Sub 3-165 | m/z = 480.12 (C₃₇H₁₇D₅BrN = 481.45) |
| Sub 3-166 | m/z = 525.11 (C₃₄H₂₄BrN = 526.48) |
| Sub 3-167 | m/z = 575.12 (C₃₈H₂₆BrN = 576.54) |
| Sub 3-169 | m/z = 551.12 (C₃₆H₂₅BrN = 552.52) |
| Sub 3-171 | m/z = 616.15 (C₄₀H₂₉BrN₂ = 617.59) |
| Sub 3-178 | m/z = 627.16 (C₄₂H₃₀BrN = 628.61) |
| Sub 3-182 | m/z = 489.07 (C₃₀H₂₀BrNO = 490.4) |
| Sub 3-184 | m/z = 555.07 (C₃₄H₂₂BrNS = 556.52) |
| Sub 3-185 | m/z = 657.11 (C₄₂H₂₈BrNS = 658.66) |
| Sub 3-186 | m/z = 591.16 (C₃₉H₃₀BrN = 592.58) |
| Sub 3-187 | m/z = 631.19 (C₄₂H₃₄BrN = 632.65) |
| Sub 3-188 | m/z = 505.05 (C₃₀H₂₀BrNS = 506.46) |
| Sub 3-190 | m/z = 581.08 (C₃₆H₂₄BrNS = 582.56) |
| Sub 3-193 | m/z = 611.04 (C₃₆H₂₇BrNS₂ = 612.6) |
| Sub 3-194 | m/z = 631.1 (C₄₀H₂₆BrNS = 632.62) |
| Sub 3-204 | m/z = 539.09 (C₃₄H₂₂BrNO = 540.46) |
| Sub 3-206 | m/z = 615.12 (C₄₀H₂₆BrNO = 616.56) |
| Sub 3-207 | m/z = 641.14 (C₄₂H₂₈BrNO = 642.6) |
| Sub 3-211 | m/z = 565.14 (C₃₇H₂₈BrN = 566.54) |
| Sub 3-212 | m/z = 591.16 (C₃₉H₃₀BrN = 592.58) |

[symbol]

indicates data missing or illegible when filed

Illustration of Synthesis of Sub 4

Synthesis methods for Sub 4 of Reaction Formula 8 may be the same as, but are not limited to, the synthesis methods for Sub 2. Compounds belonging to Sub 4 may be the same as, but are not limited to, the compounds of Sub 2.

Synthesis of Final Product 2

After Sub 3 (1 equivalent) was dissolved with toluene in a round bottom flask, Sub 4 (1 equivalent), Pd₂(dba)₃ (0.03 equivalent), (t-Bu)₃P (0.1 equivalent), and NaOt-Bu (3 equivalents) were stirred at 100° C. When the reaction was completed, extraction was performed with CH₂Cl₂ and water, and an organic layer was dried with MgSO₄ and concentrated. Afterwards, a resultant compound was subjected to silica gel column chromatography and recrystallization, thereby creating Final Product 2.

Some compounds according to the present disclosure were prepared by synthesis methods disclosed in Korean Patent Nos. 10-1668448 (patented on Oct. 17, 2016) and 10-1789998 (patented on Oct. 19, 2017) of the applicant.

### 1. Synthesis Example of 10-37

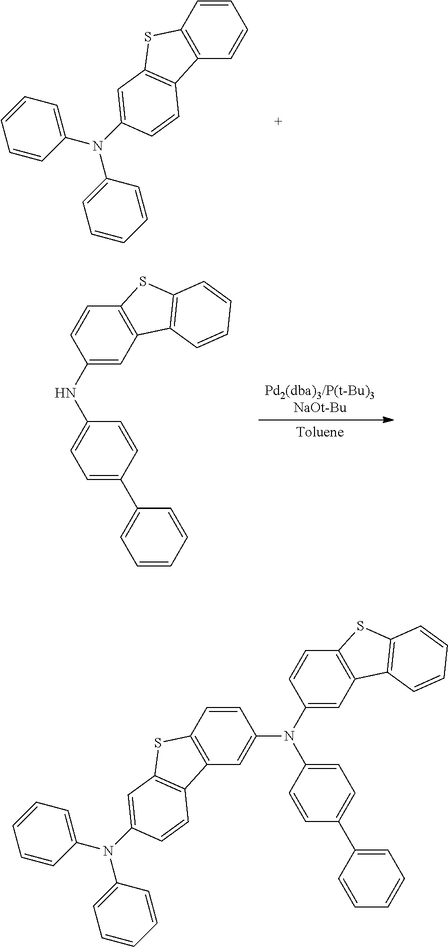

N-([1,1′-biphenyl]-4-yl)dibenzo[b,d]thiophen-2-amine (4.32 g, 12.29 mmol), Pd₂(dba)₃ (0.34 g, 0.37 mmol) P(t-Bu)₃ (0.25 g, 1.23 mmol), NaOt-Bu (3.54 g, 36.87 mmol), and toluene (125 ml) were added to N,N-diphenyldibenzo[b,d]thiophen-3-amine (5.29 g, 12.29 mmol) created in the above synthesis, and then a product 6.81 g (yield: 79%) was created using the synthesis method.

### 2. 7-8 Synthesis Example

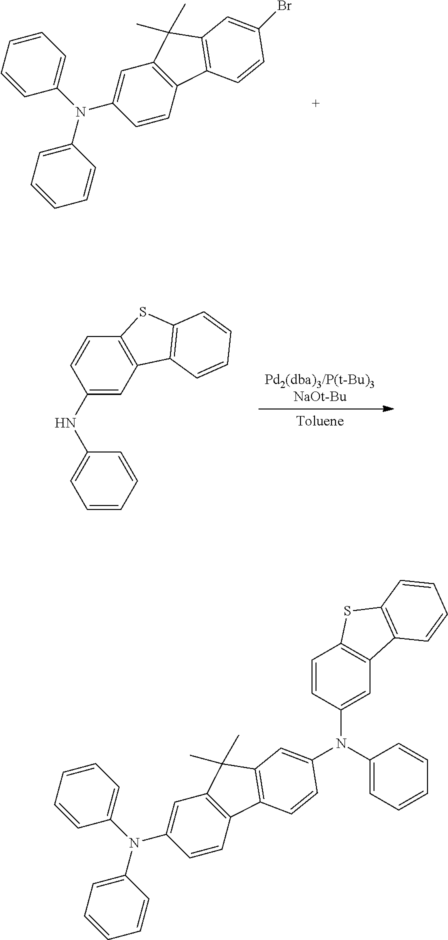 [SMILES: CC1(C)c2cc(Br)ccc2-c2ccc(N(c3ccccc3)c3ccccc3)cc21 ; c1ccc(Nc2ccc3sc4ccccc4c3c2)cc1 ; "Pd2(dba)3/P(t-Bu)3NaOt-Bu" ; "Toluene" ; CC1(C)c2cc(N(c3ccccc3)c3ccccc3)ccc2-c2ccc(N(c3ccccc3)c3ccc4sc5ccccc5c4c3)cc21]

N-phenyldibenzo[b,d]thiophen-2-amine (5 g, 18.2 mmol), Pd₂(dba)₃ (0.5 g, 0.55 mmol) P(t-Bu)₃ (0.23 g, 1.1 mmol), NaOt-Bu (5.3 g, 54.6 mmol), and toluene (100 ml) were added to 7-bromo-9,9-dimethyl-N,N-diphenyl-9H-fluoren-2-amine (8 g, 18.2 mmol) created by the above synthesis, and then a product 8.9 g (yield: 76%) was created using the above 10-37 synthesis method.

### 3. 10-176 Synthesis Example

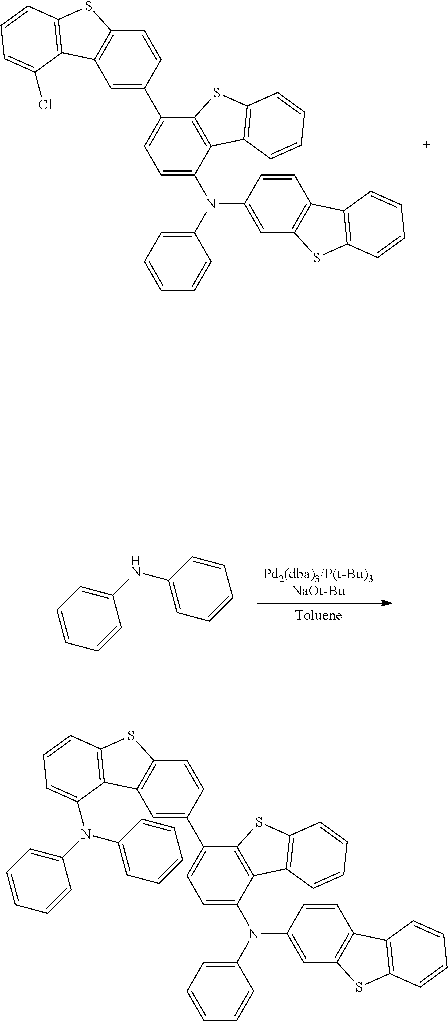 [SMILES: Clc1cccc2sc3ccc(-c4ccc(N(c5ccccc5)c5ccc6c(c5)sc5ccccc56)c5c4sc4ccccc45)cc3c12 ; c1ccc(Nc2ccccc2)cc1 ; "Pd2(dba)3/P(t-Bu)3NaOt-Bu" ; "Toluene" ; c1ccc(N(c2ccccc2)c2cccc3sc4ccc(-c5ccc(N(c6ccccc6)c6ccc7c(c6)sc6ccccc67)c6c5sc5ccccc56)cc4c23)cc1]

Diphenylamine (6.27 g, 37.08 mmol), Pd₂(dba)₃ (1.02 g, 1.11 mmol) P(t-Bu)₃ (7.50 g, 37.08 mmol), NaOt-Bu (7.13 g, 74.15 mmol), and toluene (371 ml) were added to 9-chloro-N-(dibenzo[b,d]thiophen-3-yl)-N-phenyl-[2,4′-bidibenzo[b,d]thiophen]-1′-amine (25 g, 37.08 mmol) created by the above synthesis, and then a product 8.9 g (yield: 72%) was created using the above 10-37 synthesis method.

### 4. 12-1 Synthesis Example

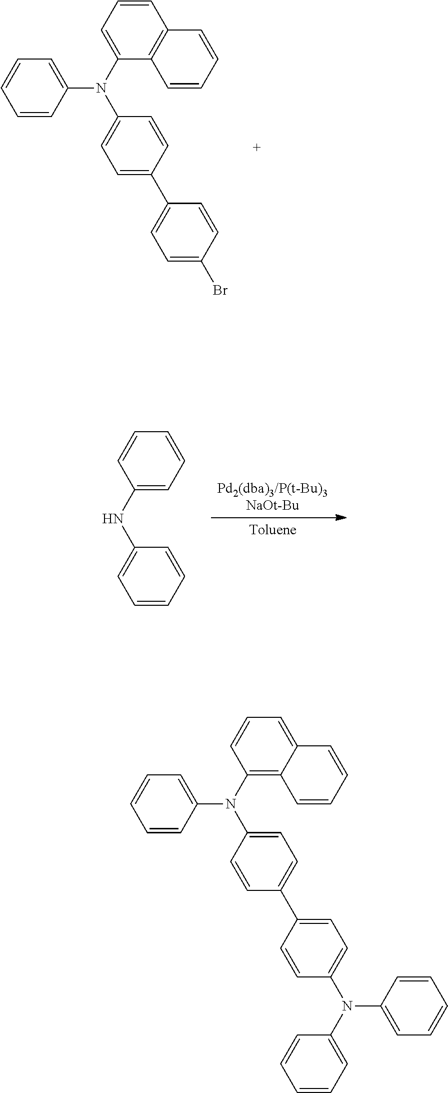 [SMILES: Brc1ccc(-c2ccc(N(c3ccccc3)c3cccc4ccccc34)cc2)cc1 ; c1ccc(Nc2ccccc2)cc1 ; "Pd2(dba)3/P(t-Bu)3NaOt-Bu" ; "Toluene" ; c1ccc(N(c2ccccc2)c2ccc(-c3ccc(N(c4ccccc4)c4cccc5ccccc45)cc3)cc2)cc1]

Diphenylamine (9.39 g, 55.51 mmol), Pd₂ (dba) 3 (1.52 g, 1.67 mmol) P(t-Bu)₃ (11.23 g, 55.51 mmol), NaOt-Bu (10.67 g, 111.02 mmol), and toluene (555 ml) were added to N-(4′-bromo-[1,1′-biphenyl]-4-yl)-N-phenylnaphthalen-1-amine (25 g, 55.51 mmol) created by the above synthesis, and then a product 21.77 g (yield: 81%) was created using the above 10-37 synthesis method.

In the meantime, FD-MS values of the compounds 7-1 to 12-63 prepared according to Synthesis Examples of the present disclosure as described above are illustrated in Table 5 below.

TABLE 5

| Compound | FD-MS | Compound | FD-MS |
|---|---|---|---|
| 7-1 | m/z = 760.29(C₅₅H₄₀N₂S = 761) | 7-2 | m/z = 744.31(C₅₅H₄₀N₂O = 744.94) |
| 7-3 | m/z = 680.32(C₅₁H₄₀N₂ = 680.9) | 7-4 | m/z = 720.35(C₅₄H [symbol] N₂ = 720.96) |
| 7-5 | m/z = 528.26(C₃₉H₃₂N₂ = 528.7) | 7-6 | m/z = 578.27(C₄₃H₃₄N₂ = 578.76) |
| 7-7 | m/z = 680.32(C₅₁H₄₀N₂ = 680.9) | 7-8 | m/z = 634.24(C₄₃H₃₄N₂S = 634.84) |
| 7-9 | m/z = 618.27(C₄₅H₃₄N₂O = 618.78) | 7-10 | m/z = 634.24(C₄₅H₃₄N₂S = 634.34) |
| 7-11 | m/z = 618.27(C₄₅H₃₄N₂O = 618.78) | 7-12 | m/z = 652.29(C₄₉H₃₆N₂ = 652.84) |
| 7-13 | m/z = 720.35(C₅₄H₄₄N₂ = 720.96) | 7-14 | m/z = 760.38(C₅₇H₄₈N₂ = 761.03) |
| 7-15 | m/z = 693.31(C₅₁H₃₉N₃ = 693.89) | 7-16 | m/z = 584.32(C₄₃H₄₀N₂ = 584.81) |
| 7-17 | m/z = 752.32(C₅₇H₄₀N₂ = 752.96) | 7-18 | m/z = 742.33(C₅₆H₄₂N₂ = 742.97) |
| 7-19 | m/z = 758.28(C₅₅H₃₈N₂S = 758.98) | 7-20 | m/z = 768.35(C₅₈H₄₄N₂ = 769) |
| 7-21 | m/z = 650.27(C₄₉H₃₄N₂ = 650.83) | 7-22 | m/z = 766.33(C₅₈H₄₂N₂ = 766.99) |
| 7-23 | m/z = 750.3(C₅₇H₃₈N₂ = 750.95) | 7-24 | m/z = 646.24(C₄₉H₃₀N₂ = 646.79) |
| 7-25 | m/z = 817.35(C₆₁H₄₃N₃ = 818.04) | 7-26 | m/z = 756.26(C₅₅H₃₆N₂S = 756.97) |
| 7-27 | m/z = 830.29(C₆₁H₃₈N₂O₂ = 830.99) | 7-28 | m/z = 815.33(C₆₁H₄₁N₃ = 816.02) |
| 7-29 | m/z = 694.33(C₅₂H₄₂N₂ = 694.92) | 7-30 | m/z = 668.28(C₄₉H₃₆N₂O = 668.84) |
| 7-31 | m/z = 710.28(C₅₁H₃₈N₂S = 710.94) | 7-32 | m/z = 684.26(C₄₉H₃₆N₂S = 684.9) |
| 7-33 | m/z = 668.28(C₄₉H₃₆NO = 668.84) | 7-34 | m/z = 604.29(C₄₅H₃₆N₂ = 604.8) |
| 7-35 | m/z = 630.3(C₄₇H₃₈N₂ = 630.84) | 7-36 | m/z = 708.29(C₅₂H₃₇FN₂ = 708.88) |
| 7-37 | m/z = 830.37(C₆₃H₄₆N₂ = 831.08) | 7-38 | m/z = 808.29(C₅₉H₄₀N₂S = 809.04) |
| 7-39 | m/z = 844.38(C₆₄H₄₈N₂ = 845.1) | 7-40 | m/z = 834.31(C₆₁H₄₂N₂S = 835.08) |
| 7-41 | m/z = 742.3(C₅₅H₃₈N₂O = 742.92) | 7-42 | m/z = 968.41(C₇₄H₅₂N₂ = 969.24) |
| 7-43 | m/z = 690.3(C₅₂H₃₈N₂ = 690.89) | 7-44 | m/z = 802.33(C₆₁H₄₂N₂ = 803.02) |
| 7-45 | m/z = 816.31(C₆₁H₄₀N₂O = 817) | 7-46 | m/z = 708.26(C₅₁H₃₆N₂S = 708.92) |
| 7-47 | m/z = 832.29(C₆₁H₄₀N₂S = 833.07) | 7-48 | m/z = 694.3(C₅₁H₃₈N₂O = 694.88) |
| 7-49 | m/z = 818.37(C₆₂H₄₆N₂ = 819.06) | 7-50 | m/z = 802.33(C₆₁H₄₂N₂ = 803.02) |
| 7-51 | m/z = 769.35(C₅₇H₄₃N₃ = 769.99) | | |
| 10-1 | m/z = 618.21(C₄₄H₃₀N₂S = 618.80) | 10-2 | m/z = 700.20(C₄₈H₃₂N₂S₂ = 700.92) |
| 10-3 | m/z = 708.22(C₅₀H₃₂N₂OS = 708.88) | 10-4 | m/z = 692.25(C₅₀H₃₂N₂O₂ = 692.82) |
| 10-5 | m/z = 742.30(C₅₅II_{3S}N₂O = 742.92) | 10-6 | m/z = 654.27(C₄₈II₃₄N₂O = 654.81) |
| 10-7 | m/z = 700.20(C₄₈H₃₂N₂S₂ = 700.92) | 10-8 | m/z = 760.25(C₅₄H₃₆N₂OS = 760.96) |
| 10-9 | m/z = 730.16(C₄₈H₃₀N₂S₃ = 730.96) | 10-10 | m/z = 734.24(C₅₂H₃₄N₂OS = 734.92) |
| 10-11 | m/z = 872.32(C₆₄H₄₄N₂S = 873.13) | 10-12 | m/z = 718.26(C₅₂H₅₄N₂O₂ = 718.86) |
| 10-13 | m/z = 568.20(C₄₀H₂₈N₂S = 568.74) | 10-14 | m/z = 624.17(C₄₂H₂₈N₂S₂ = 624.82) |
| 10-15 | m/z = 658.21(C₄₆H₃₀N₂OS = 658.82) | 10-16 | m/z = 730.30(C₅₄H₃₈N₂O = 730.91) |
| 10-17 | m/z = 698.20(C₄₈H₃₀N₂O₂S = 698.84) | 10-18 | m/z = 624.17(C₄₂H₂₈N₂S₂ = 624.82) |
| 10-19 | m/z = 700.20(C₄₈H₃₂N₂S = 700.92) | 10-20 | m/z = 708.22(C₅₆H [symbol] N₂OS = 708.88) |
| 10-21 | m/z = 750.22(C₅₂H₃₄N₂S₂ = 750.98) | 10-22 | m/z = 776.23(C₅₄H₃₆N₂S₂ = 777.02) |
| 10-23 | m/z = 867.24(C₃₀H₃₇N₃OS₂ = 868.09) | 10-24 | m/z = 759.27(C₅₄H [symbol] N₃S = 759.97) |
| 10-25 | m/z = 608.19(C₄₂H₂₈N₂OS = 608.76) | 10-26 | m/z = 608.19(C₄₂H₂₈N₂OS = 608.76) |
| 10-27 | m/z = 692.25(C₅₆H₃₂N₂O₂ = 692.82) | 10-28 | m/z = 894.20(C₆₀H₃₄N₂O₃S₂ = 895.06) |
| 10-29 | m/z = 618.21(C₄₄H₃₀N₃S = 618.90) | 10-30 | m/z = 700.20(C₄₈H₃₂N₂S₂ = 700.92) |
| 10-31 | m/z = 780.17(C₅₂H₃₂N₂S₃ = 781.02) | 10-32 | m/z = 734.24(C₅₂H₃₄N₂OS = 734.92) |
| 10-33 | m/z = 834.31(C₆₁H₄₂N₂S = 835.08) | 10-34 | m/z = 700.20(C₄₈H₃₂N₂S₂ = 700.92) |
| 10-35 | m/z = 724.25(C₅₁H₃₆N₂OS = 724.92) | 10-36 | m/z = 624.17(C₄₂H₂₈N₂S₂ = 624.82) |
| 10-37 | m/z = 700.20(C₄₈H₃₂N₂S₂ = 700.92) | 10-38 | m/z = 700.20(C₄₈H₃₂N₂S₂ = 700.92) |
| 10-39 | m/z = 674.19(C₄₆H₃₀N₂S₂ = 674.88) | 10-40 | m/z = 700.20(C₄₈H₃₂N₂S₂ = 700.92) |
| 10-41 | m/z = 608.19(C₄₂H₂₈N₂OS = 608.76) | 10-42 | m/z = 684.22(C₄₈H₃₂N₂OS = 684.86) |
| 10-43 | m/z = 684.22(C₄₈H₃₂N₂OS = 684.86) | 10-44 | m/z = 624.17(C₄₂H₂₈N₂S₂ = 624.82) |
| 10-45 | m/z = 674.19(C₄₆H₃₀N₂S₂ = 674.88 | 10-46 | m/z = 700.20(C₄₈H₃₂N₂S₂ = 700.92) |
| 10-47 | m/z = 826.25(C₅₈H₃₈N₂S₂ = 827.08) | 10-48 | m/z = 608.19(C₄₂H₂₈N₂OS = 608.76) |
| 10-49 | m/z = 624.17(C₄₂H₂₈N₂S₂ = 624.82) | 10-50 | m/z = 684.22(C₄₈H₃₂N₂OS = 684.86) |
| 10-51 | m/z = 700.20(C₄₈H₃₂N₂S₂ = 700.92) | 10-52 | m/z = 684.22(C₄₈H₃₂N₂OS = 684.86) |
| 10-53 | m/z = 730.16(C₄₈H₃₀N₂S₃ = 730.96) | 10-54 | m/z = 826.25(C₅₈H₃₈N₂S₂ = 827.08) |
| 10-55 | m/z = 806.19(C₅₄H₃₄N₂S₃ = 807.06 | 10-56 | m/z = 674.19(C₄₆H₃₀N₂S₂ = 674.88) |
| 10-57 | m/z = 674.19(C₄₆H₃₀N₂S₂ = 674.88) | 10-58 | m/z = 674.19(C₄₆H₃₀N₂S₂ = 674.88) |
| 10-59 | m/z = 674.19(C₄₆H₃₀N₂S₂ = 674.88) | 10-60 | m/z = 674.19(C₄₆H₃₀N₂S₂ = 674.88) |
| 10-61 | m/z = 638.19(C [symbol] H₃₀N₂S₂ = 638.85) | 10-62 | m/z = 638.19(C₄₃H₃₀N₂S₂ = 638.85) |
| 10-63 | m/z = 638.19(C₄₃H₃₀N₂S₂ = 638.55) | 10-64 | m/z = 622.21(C₄₅H₃₀N₂S = 622.79) |
| 10-65 | m/z = 688.20(C₄₇H₃₃N₂S₂ = 688.51) | 10-66 | m/z = 700.20(C₄₈H₃₂N₂S₂ = 700.92) |
| 10-67 | m/z = 789.23(C₅₄H₃₄N₃S₂ = 790.02) | 10-68 | m/z = 638.19(C₄₅H₃₀N₂S₂ = 638.85) |
| 10-69 | m/z = 638.19(C [symbol] H₃₀N₂S₂ = 638.55) | 10-70 | m/z = 642.16(C₄₂H₂₇N₂S₂ = 642.81) |
| 10-71 | m/z = 638.19(C₄₃H₃₀N₂S₂ = 638.55) | 10-72 | m/z = 714.22(C₄₉H₃₄N₂S₂ = 714.95) |
| 10-73 | m/z = 716.20(C₄₈H₃₂N₂OS₂ = 716.92) | 10-74 | m/z = 670.24(C₄₈H₃₄N₂S = 670.87) |
| 10-75 | m/z = 634.24(C₄₅H₃₄N₂S = 634.84) | 10-76 | m/z = 759.27(C₅₄H₃₇N₃S = 759.97) |
| 10-77 | m/z = 674.19(C₄₆H₃₀N₂S₂ = 674.88) | 10-78 | m/z = 708.22(C₅₀H₃₂N₂OS = 708.88) |
| 10-79 | m/z = 674.19(C₄₆H₃₀N₂S₂ = 674.88) | 10-80 | m/z = 748.22(C₅₂H₃₂N₂O₂S = 748.90) |
| 10-81 | m/z = 821.29(C [symbol] H₃₅D₅N₂S₂ = 822.11) | 10-82 | m/z = 730.16(C₄₈H₃₀N₂S₃ = 730.96) |
| 10-83 | m/z = 730.16(C₄₅H₃₀N₂S₃ = 730.96) | 10-84 | m/z = 714.18(C₄₈H₃₀N₂OS₂ = 714.90) |
| 10-85 | m/z = 882.22(C₆₀H₃₈N₂S₃ = 883.16) | 10-86 | m/z = 624.17(C₄₂H₂₈N₂S₂ = 624.82) |
| 10-87 | m/z = 700.20(C₄₈H₃₂N₂S₂ = 700.92) | 10-88 | m/z = 674.19(C₄₆H [symbol] N₂S₂ = 674.88) |
| 10-89 | m/z = 674.19(C₄₆H₃₀N₂S₃ = 674.88) | 10-90 | m/z = 708.22(C₅₆H₃₂N₂OS = 708.88) |
| 10-91 | m/z = 759.27(C [symbol] H [symbol] N₂S = 759.97) | 10-92 | m/z = 674.19(C₄₆H₃₀N₂S₂ = 674.88) |
| 10-93 | m/z = 688.20(C₄₇H₃₂N₂S₂ = 688.91) | 10-94 | m/z = 760.25(C₅₄H₃₆N₂OS = 760.96) |
| 10-95 | m/z = 759.27(C₅₄H₃₇N₃S = 759.97) | 10-96 | m/z = 608.19(C₄₂H₂₈N₂OS = 608.76) |
| 10-97 | m/z = 684.22(C₄₈H₃₂N₂OS = 684.86) | 10-98 | m/z = 622.21(C₄₃H₃₆N₂OS = 622.79) |
| 10-99 | m/z = 760.25(C₅₄H₃₆N₂OS = 760.96) | 10-100 | m/z = 658.21(C₄₆H₃₀N₂OS = 658.82) |
| 10-101 | m/z = 658.21(C₄₆H₃₀N₂OS = 658.82) | 10-102 | m/z = 692.25(C₅₀H₃₂N₂O₂ = 692.82) |
| 10-103 | m/z = 628.25(C₄₆H₃₂N₂O = 628.78) | 10-104 | m/z = 693.28(C [symbol] H₃₅N₂O = 692.85) |
| 10-105 | m/z = 723.23(C₅₀H₃₃N₃OS = 723.89) | 10-106 | m/z = 749.29(C₅₄H [symbol] D₅N₂S = 749.99) |
| 10-107 | m/z = 700.20(C₄₈H₃₂N₂S₂ = 700.92) | 10-108 | m/z = 674.19(C₄₆H₃₆N₂S₂ = 674.88) |
| 10-109 | m/z = 684.22(C [symbol] H₃₂N₂OS = 684.86) | 10-110 | m/z = 724.25(C₅₁H₃₈N₂OS = 724.92) |
| 10-111 | m/z = 674.19(C₄₆H₃₀N₂S₂ = 674.88) | 10-112 | m/z = 674.19(C₄₆H₃₆N₂OS = 674.88) |
| 10-113 | m/z = 780.17(C₅₂H₃₂N₂S₃ = 781.02) | 10-114 | m/z = 684.22(C₄₈H₃₂N₂OS = 684.86) |
| 10-115 | m/z = 684.22(C₄₈H₃₂N₂OS = 684.86) | 10-116 | m/z = 698.20(C₄₈H₃₆N₂O₂S = 698.84) |
| 10-117 | m/z = 618.26(C₄₂H₂₈D₁₀N₂OS = 618.82) | 10-118 | m/z = 757.27(C₅₄H₃₅N₃O₂ = 757.89) |
| 10-119 | m/z = 668.25(C₄₈H₃₂N₂O₂ = 668.80) | 10-120 | m/z = 720.26(C₅₂H₃₆N₂S = 720.93) |
| 10-121 | m/z = 670.24(C₄₈H₃₄N₂S = 670.87) | 10-122 | m/z = 700.20(C₄₈H₃₂N₂S = 700.92) |
| 10-123 | m/z = 674.19(C₄₆H₃₀N₂S₂ = 674.38) | 10-124 | m/z = 700.20(C₄₈H₃₂N₂S = 700.92) |
| 10-125 | m/z = 608.19(C₄₂H₂₈N₂OS = 608.76) | 10-126 | m/z = 860.25(C₅₈H₄₀N₂O₂S₂ = 861.09) |
| 10-127 | m/z = 798.27(C [symbol] H₃₈N₂OS = 799.00) | 10-128 | m/z = 734.33(C₅₄H₄₂N₂O = 734.94) |
| 10-129 | m/z = 742.26(C₅₄H₃₄N₂O₂ = 742.88) | 10-130 | m/z = 945.37(C [symbol] H [symbol] N [symbol] O = 946.17) |
| 10-131 | m/z = 670.24(C₄₈H₃₄N₂S = 670.87) | 10-132 | m/z = 624.17(C₄₂H₂₈N₂S = 624.82) |
| 10-133 | m/z = 700.20(C₄₈H₃₂N₂S₂ = 700.92) | 10-134 | m/z = 724.20(C₃₆H₃₂N₂S₂ = 724.94) |
| 10-135 | m/z = 816.26(C₃₇H₄₀N₂S₂ = 817.08) | 10-136 | m/z = 760.25(C₅₄H₄₆N₂OS = 760.96) |
| 10-137 | m/z = 674.19(C₄₆H₃₀N₂S₂ = 674.38) | 10-138 | m/z = 730.16(C₄₈H₃₆N₂S₃ = 730.96) |
| 10-139 | m/z = 881.29(C [symbol] H₄₃N₃S₂ = 882.16) | 10-140 | m/z = 704.28(C₅₂H₃₆N₂O = 704.87) |
| 10-141 | m/z = 608.19(C₄₂H₂₈N₂OS = 608.76) | 10-142 | m/z = 682.23(C₄₈H₃₆N₂O₃ = 682.78) |
| 10-143 | m/z = 670.24(C₄₈H₃₄N₂S = 670.87) | 10-144 | m/z = 776.23(C₅₄H₃₆N₂S₂ = 777.02) |
| 10-145 | m/z = 674.19(C₄₆H₃₀N₂S₂ = 674.88) | 10-146 | m/z = 759.27(C₅₄H₃₇N₃S = 759.97) |
| 10-147 | m/z = 758.24(C₅₄H₃₄N₂OS = 758.94) | 10-148 | m/z = 806.28(C [symbol] H [symbol] N₂S = 807.03) |
| 10-149 | m/z = 823.30(C₅₉H₃₈FN₃O = 823.97) | 10-150 | m/z = 743.29(C₅₄H₃₇N₃O = 743.91) |
| 10-151 | m/z = 998.33(C [symbol] H₄₆N₂OS = 999.24) | 10-152 | m/z = 700.20(C [symbol] H₃₂N₂S₂ = 700.32) |
| 10-153 | m/z = 674.19(C₄₆H₃₆N₂S₂ = 674.88) | 10-154 | m/z = 674.19(C₄₆H₃₀N₂S₂ = 674.88) |
| 10-155 | m/z = 674.19(C₄₆H₃₆N₂S₂ = 674.88) | 10-158 | m/z = 674.19(C₄₆H₃₀N₂S₂ = 674.88) |
| 10-157 | m/z = 762.29(C₅₀H₄₆N₂SSi₂ = 763.16) | 10-158 | m/z = 624.17(C₄₂H₂₈N₂S₂ = 624.82) |
| 10-159 | m/z = 784.25(C₅₆H₃₆N₂OS = 784.98) | 10-160 | m/z = 810.27(C₃₈H₃₈N₂OS = 811.02) |
| 10-161 | m/z = 860.29(C [symbol] H₄₀N₂OS = 861.08) | 10-162 | m/z = 708.22(C₅₀H [symbol] N₂OS = 708.88) |
| 10-163 | m/z = 742.26(C₅₄H₃₄N₂O₂ = 742.88) | 10-164 | m/z = 828.26(C₃₈H₄₀N₂S₂ = 829.09) |
| 10-165 | m/z = 708.22(C₅₉H₃₂N₂OS = 708.88) | 10-166 | m/z = 724.20(C₅₀H₃₂N₂S₂ = 724.94) |
| 10-167 | m/z = 834.27(C [symbol] H₃₈N₂OS = 835.04) | 10-168 | m/z = 768.28(C₅₀H₃₆N₂O₂ = 768.92) |
| 10-169 | m/z = 830.19(C₅₆H₃₄N₂S₃ = 831.08) | 10-170 | m/z = 810.27(C₅₈H₃₈N₂OS = 811.02) |
| 10-171 | m/z = 810.31(C₅₉H₄₂N₂S = 811.06) | 10-172 | m/z = 692.25(C₅₆H₃₂N₂O₂ = 692.82) |
| 10-173 | m/z = 758.24(C₅₄H₃₄N₂OS = 758.94) | 10-174 | m/z = 783.27(C₅₆H₃₇N₃S = 783.99) |
| 10-175 | m/z = 808.25(C₅₈H₃₆N₂OS = 809.00) | 10-176 | m/z = 806.19(C₅₄H [symbol] N₂S₃ = 807.06) |
| 10-177 | m/z = 764.2(C [symbol] H₃₂N₂OS₂ = 764.96) | 10-178 | m/z = 684.22(C₄₈H₃₂N₂OS = 684.86) |
| 10-179 | m/z = 774.27(C [symbol] H₃₈N₂OS = 774.98) | 10-180 | m/z = 684.22(C₄₈H₃₂N₂OS = 684.86) |
| 10-181 | m/z = 658.21(C₄₆H₃₀N₂OS = 658.82) | 10-182 | m/z = 700.2(C₄₈H₃₂N₂S₂ = 700.92) |
| 10-183 | m/z = 700.2(C₄₈H₃₂N₂S₂ = 700.92) | 10-184 | m/z = 700.2(C₄₈H₃₂N₂S₂ = 700.92) |
| 10-185 | m/z = 684.22(C₄₈H₃₂N₂OS = 684.86) | 10-186 | m/z = 684.22(C₄₈H₃₂N₂OS = 684.86) |
| 10-187 | m/z = 724.25(C₅₁H₃₆N₂OS = 724.92) | 10-188 | m/z = 832.31(C₆₁H₄₀N₂O₂ = 833) |
| 10-189 | m/z = 861.37(C₆₃H₄₇N₂O = 862.09) | | |
| 12-1 | m/z = 690.30(C₅₂H₃₈N₂ = 690.87) | 12-2 | m/z = 790.33(C [symbol] H₄₂N₂ = 790.99) |
| 12-3 | m/z = 740.32(C₅₆H₄₀N₂ = 740.93) | 12-4 | m/z = 840.35(C₆₄H₄₈N₂ = 841.09) |
| 12-5 | m/z = 691.30(C₅₁H₃₇N₃ = 691.86) | 12-6 | m/z = 688.29(C₅₂H₃₆N₂ = 688.86) |
| 12-7 | m/z = 700.37(C₅₂H₂₈D₁₉N₂ = 700.93) | 12-8 | m/z = 650.35(C₄₈H₃₆D₁₀N₂ = 650.87) |
| 12-9 | m/z = 922.40(C [symbol] H [symbol] N [symbol] = 923.15) | 12-10 | m/z = 730.33(C₅₅H₄₂N₂ = 730.94) |
| 12-11 | m/z = 832.38(C₆₃H₄₈N₂ = 833.07) | 12-12 | m/z = 761.38(C₅₇H₃₉D₅N₂ = 762.00) |
| 12-13 | m/z = 806.37(C₆₁H₄₆N₂ = 807.03) | 12-14 | m/z = 875.35(C₆₄H₄₈N₂S = 877.14) |
| 12-15 | m/z = 872.41(C₆₆H₅₂N₂ = 873.13) | 12-16 | m/z = 770.37(C₅₈H₄₉N₂ = 771.00) |
| 12-17 | m/z = 952.48(C₇₂H₆₀N₂ = 953.26) | 12-18 | m/z = 828.35(C [symbol] H₄₄N₂ = 829.04) |
| 12-19 | m/z = 910.34(C₆₇H₄₆N₂S = 911.16) | 12-20 | m/z = 863.33(C₆₂H₄₃N₃S = 864.11) |
| 12-21 | m/z = 804.35(C₆₁H₄₄N₂ = 805.02) | 12-22 | m/z = 970.39(C₇₃H₅₉N₂O = 971.19) |
| 12-23 | m/z = 981.41(C₇₄H₅₁N₃ = 982.22) | 12-24 | m/z = 968.41(C₇₄H₅₂N₂ = 969.22) |
| 12-25 | m/z = 878.37(C₆₇H₄₆N₂ = 879.10) | 12-26 | m/z = 815.31(C₆₁H₄₉N₂O = 816.98) |
| 12-27 | m/z = 805.35(C₆₀H₄₃N₃ = 806.00) | 12-28 | m/z = 885.32(C₆₄H₄₃N₃S = 886.11) |
| 12-29 | m/z = 805.35(C₆₀H₄₃N₃ = 806.00) | 12-30 | m/z = 1050.47(C₇₈H₅₈N₄ = 1051.32) |
| 12-31 | m/z = 686.26(C₅₀H₃₆N₂S = 696.80) | 12-32 | m/z = 695.26(C₅₀H₃₆N₂S = 696.90) |
| 12-33 | m/z = 822.31(C₆₀H₄₂N₂S = 823.05) | 12-34 | m/z = 746.28(C₅₄H₃₈N₂S = 746.96) |
| 12-35 | m/z = 700.20(C₄₈H₃₂N₂S₂ = 700.91) | 12-38 | m/z = 800.23(C₅₆H₃₆N₂S₂ = 801.03) |
| 12-37 | m/z = 852.26(C₆₀H₄₀N₂S₂ = 853.10) | 12-38 | m/z = 992.23(C₆₈H₄₄N₂S₂ = 993.22) |
| 12-39 | m/z = 912.18(C₆₀H₃₆N₂S₄ = 913.20) | 12-40 | m/z = 852.26(C₆₀H₄₀N₂S₂ = 85310) |
| 12-41 | m/z = 806.33(C₆₀H₄₂N₂O = 806.99) | 12-42 | m/z = 768.28(C₅₆H₃₆N₂O₂ = 768.50) |
| 12-43 | m/z = 920.34(C [symbol] H₄₄N₂O₂ = 921.09) | 12-44 | m/z = 684.22(C₄₈H₃₂N₂OS = 684.85) |
| 12-45 | m/z = 970.43(C₇₄H₅₄N₂ = 971.23) | 12-46 | m/z = 947.42(C₇₁H₅₃N₃ = 948.20) |
| 12-47 | m/z = 829.35(C₆₂H₄₃N₃ = 830.02) | 12-48 | m/z = 860.29(C₆₂H₄₉N₂OS = 861.06) |
| 12-49 | m/z = 664.29(C₅₀H₃₅N₂ = 664.83) | 12-50 | m/z = 956.41(C₇₃H₅₂N₂ = 957.21) |
| 12-51 | m/z = 829.35(C₆₂H₄₃N₃ = 830.02) | 12-52 | m/z = 911.33(C₆₈H₃₄N₃S = 912.15) |
| 12-53 | m/z = 664.29(C₅₀H₃₆N₂ = 664.83) | 12-54 | m/z = 776.23(C₅₄H₃₆N₂S₂ = 777.01) |
| 12-55 | m/z = 744.28(C₅₄H₃₆N₂O₂ = 744.88) | 12-56 | m/z = 894.37(C₆₆H₄₆N₄ = 895.10) |
| 12-57 | m/z = 776.23(C₅₁H₃₆N₂S₂ = 777.01) | 12-58 | m/z = 844.31(C₆₂H₄₀N₂O₂ = 844.99) |
| 12-59 | m/z = 664.29(C₅₀H₃₆N₂ = 664.83) | 12-60 | m/z = 844.31(C₆₂H₄₀N₂O₂ = 844.99) |
| 12-61 | m/z = 640.29(C₄₈H₃₆N₂ = 640.83) | 12-62 | m/z = 604.29(C₄₅H₃₆N₂ = 604.8) |
| 12-63 | m/z = 578.24(C₄₂H₃₀N₂O = S78.72) | 12-64 | m/z = 654.27(C₄₈H₃₄N₂O = 654.81) |
| 12-66 | m/z = 730.3(C₅₄H₃₈N₂O = 730.91) | 12-68 | m/z = 770.33(C₅₇H₄₂N₂O = 770.98) |
| 12-70 | m/z = 668.25(C₄₈H₃₂N₂O₂ = 668.8) | | |

[symbol]

indicates data missing or illegible when filed

Evaluation of Fabrication of Organic Electronic Device

When the organic electronic device according to the present disclosure is a top-emission device and the anode is formed on the substrate before the organic material layer and the cathode are formed, the anode may be formed of not only a transparent material but also an opaque material having superior light reflectivity.

When the organic electronic device according to the present disclosure is a bottom-emission device and the anode is formed on the substrate before the organic material layer and the cathode are formed, the anode should be formed of a transparent material or, when formed of an opaque material, be provided as a thin film as thin as possible so as to be transparent.

Hereinafter, the following Examples will be proposed by fabricating a top-emission tandem organic electronic device, but embodiments of the present disclosure are not limited thereto. The tandem organic electronic device according to an embodiment of the present disclosure is fabricated such that a plurality of stacks are connected through one or more charge generation layers.

### [Example 1 to Example 45] Tandem Organic Electronic Device with Two Stacks Connected

Tandem organic electronic devices respectively including two stacks connected were fabricated with a structure of anode/hole transport region/emission layer/electron transport region/electron injection layer/cathode. Specifically, a film of N,N′-Bis(1-naphthalenyl)-N,N′-bis-phenyl-(1,1′-biphenyl)-4,4′-diamine (hereinafter, abbreviated as NPB) doped with HATCN 10% was deposited at a thickness of 10 nm on an anode formed on a glass substrate, thereby forming a hole injection layer. Afterwards, a hole transport layer I was formed by depositing a compound I (i.e., a first hole transport material) represented by Formula C or Formula D and including a radical of a compound represented by Formula A at a thickness of 15 nm, and a hole transport layer II was formed on the hole transport layer I by depositing a compound II (i.e., a second hole transport material) represented by Formula C or Formula D and including a radical of a compound represented by Formula A or Formula B at a thickness of 5 nm. DPVBi was used as a host on the hole transport layer, and BCzVBi 5% by weight was added as a dopant, thereby depositing an emission layer of 20 nm. A film of Alq3 was deposited at a thickness of 30 nm as an electron transport layer. Subsequently, charge generation layers were formed to connect two stacks. That is, an N-type charge generation layer was formed by depositing Bphen doped with Li 2%, and a P-type charge generation layer was formed by depositing NPB doped with HATCN 10%. Afterwards, a hole transport region, an emission layer, and an electron transport region were sequentially deposited as described above, thereby forming a second stack. Thereafter, an electron injection layer was formed by depositing Liq at a thickness of 1.5 nm, and then a cathode was formed by depositing Ag:Mg at a thickness of 150 run. Consequently, tandem organic electronic devices were fabricated.

Electroluminescence (EL) properties of organic electronic emission devices according to Examples and Comparative Examples fabricated as above were measured using PR-650 available from Photo Research, Inc. by applying a forward bias DC voltage to the devices, and as a result of the measurement, T95 lifespans of the devices were tested using lifetime test equipment available from Mcscience Inc. The following table illustrates the results of the fabrication and test of the devices.

### Comparative Examples 1 and 2

Tandem organic electronic devices were fabricated in the same method as Examples, except that NPB and compound 4-21 of the present disclosure were respectively used as the first hole transport material and the hole transport layer II was not used.

### Comparative Example 3

A tandem organic electronic device was fabricated in the same method as Examples, except that NPB was used as the first hole transport material and compound 6-30 of the present disclosure was used as the second hole transport material.

TABLE 6

| | 1^{st} | 2^{nd} | Driving | Current | Luminance | Efficiency | | | |
|---|---|---|---|---|---|---|---|---|---|
| | HTM | HTM | voltage | (mA/cm²) | (csd/m²) | (cd/A) | T (95) | CIE (x) | CIE (y) |
| Comp. Ex. 1 | NPB | — | 10.5 | 13.6 | 1500 | 11.0 | 60.4 | 0.131 | 0.100 |
| Comp. Ex. 2 | 4-21 | — | 10.3 | 13.4 | 1500 | 11.2 | 63.1 | 0.131 | 0.100 |
| Comp. Ex. 3 | NPB | 6-30 | 10.0 | 13.0 | 1500 | 11.5 | 67.3 | 0.131 | 0.100 |
| Ex. 1 | 1-137 | 6-30 | 9.5 | 11.0 | 1500 | 13.6 | 80.1 | 0.131 | 0.100 |
| Ex. 2 | 4-21 | 6-30 | 9.5 | 11.3 | 1500 | 13.3 | 80.3 | 0.131 | 0.100 |
| Ex. 3 | 4-45 | 6-30 | 9.4 | 11.3 | 1500 | 13.3 | 81.1 | 0.131 | 0.100 |
| Ex. 4 | 5-91 | 6-30 | 9.5 | 11.0 | 1500 | 13.6 | 80.8 | 0.130 | 0.100 |
| Ex. 5 | 6-14 | 6-30 | 9.5 | 11.0 | 1500 | 13.6 | 82.0 | 0.130 | 0.100 |
| Ex. 6 | 6-15 | 6-30 | 9.4 | 11.2 | 1500 | 13.4 | 81.5 | 0.132 | 0.100 |
| Ex. 7 | 6-32 | 6-30 | 9.4 | 11.4 | 1500 | 13.2 | 81.2 | 0.130 | 0.100 |
| Ex. 8 | 6-33 | 6-30 | 9.4 | 11.5 | 1500 | 13.1 | 81.9 | 0.131 | 0.100 |
| Ex. 9 | 6-35 | 6-30 | 9.5 | 11.5 | 1500 | 13.1 | 80.4 | 0.132 | 0.100 |
| Ex. 10 | 1-137 | 6-31 | 9.4 | 16.0 | 1500 | 13.3 | 82.1 | 0.132 | 0.100 |
| Ex. 11 | 4-21 | 6-31 | 9.4 | 16.0 | 1500 | 13.0 | 82.3 | 0.130 | 0.100 |
| Ex. 12 | 4-45 | 6-31 | 9.3 | 16.1 | 1500 | 12.9 | 83.1 | 0.132 | 0.100 |
| Ex. 13 | 5-91 | 6-31 | 9.4 | 16.0 | 1500 | 13.2 | 82.9 | 0.131 | 0.100 |
| Ex. 14 | 6-14 | 6-31 | 9.4 | 16.0 | 1500 | 13.1 | 84.1 | 0.132 | 0.100 |
| Ex. 15 | 6-15 | 6-31 | 9.3 | 16.1 | 1500 | 13.1 | 83.6 | 0.130 | 0.100 |
| Ex. 16 | 6-32 | 6-31 | 9.3 | 16.1 | 1500 | 12.9 | 83.3 | 0.131 | 0.100 |
| Ex. 17 | 6-33 | 6-31 | 9.3 | 16.1 | 1500 | 12.8 | 83.8 | 0.131 | 0.100 |
| Ex. 18 | 6-35 | 6-31 | 9.4 | 16.0 | 1500 | 12.8 | 82.6 | 0.132 | 0.100 |
| Ex. 19 | 1-137 | 11-14 | 9.1 | 1.7 | 1500 | 14.2 | 88.4 | 0.131 | 0.100 |
| Ex. 20 | 4-21 | 11-14 | 9.1 | 1.7 | 1500 | 14.0 | 88.5 | 0.131 | 0.100 |
| Ex. 21 | 4-45 | 11-14 | 9.0 | 1.7 | 1500 | 13.9 | 89.0 | 0.132 | 0.100 |
| Ex. 22 | 5-91 | 11-14 | 9.1 | 1.7 | 1500 | 14.1 | 88.9 | 0.131 | 0.100 |
| Ex. 23 | 6-14 | 11-14 | 9.1 | 1.7 | 1500 | 14.1 | 90.0 | 0.131 | 0.100 |
| Ex. 24 | 6-15 | 11-14 | 9.0 | 1.7 | 1500 | 14.0 | 89.6 | 0.131 | 0.100 |
| Ex. 25 | 6-32 | 11-14 | 9.0 | 1.7 | 1500 | 13.9 | 89.4 | 0.131 | 0.100 |
| Ex. 26 | 6-33 | 11-14 | 9.0 | 1.7 | 1500 | 13.8 | 89.7 | 0.131 | 0.100 |
| Ex. 27 | 6-35 | 11-14 | 9.1 | 1.7 | 1500 | 13.7 | 88.7 | 0.130 | 0.100 |
| Ex. 28 | 1-137 | 11-20 | 9.2 | 10.4 | 1500 | 14.4 | 86.4 | 0.130 | 0.100 |
| Ex. 29 | 4-21 | 11-20 | 9.2 | 10.6 | 1500 | 14.2 | 86.7 | 0.130 | 0.100 |
| Ex. 30 | 4-45 | 11-20 | 9.1 | 10.6 | 1500 | 14.1 | 87.2 | 0.131 | 0.100 |
| Ex. 31 | 5-91 | 11-20 | 9.2 | 10.4 | 1500 | 14.4 | 87.1 | 0.131 | 0.100 |
| Ex. 32 | 6-14 | 11-20 | 9.2 | 10.5 | 1500 | 14.3 | 88.3 | 0.130 | 0.100 |
| Ex. 33 | 6-15 | 11-20 | 9.1 | 10.6 | 1500 | 14.2 | 87.8 | 0.131 | 0.100 |
| Ex. 34 | 6-32 | 11-20 | 9.1 | 10.6 | 1500 | 14.1 | 87.5 | 0.130 | 0.100 |
| Ex. 35 | 6-33 | 11-20 | 9.1 | 10.7 | 1500 | 14.0 | 88.0 | 0.132 | 0.100 |
| Ex. 36 | 6-35 | 11-20 | 9.2 | 10.7 | 1500 | 14.0 | 86.9 | 0.131 | 0.100 |
| Ex. 37 | 1-137 | 11-21 | 9.3 | 10.8 | 1500 | 13.9 | 84.3 | 0.131 | 0.100 |
| Ex. 38 | 4-21 | 11-21 | 9.3 | 10.9 | 1500 | 13.7 | 84.6 | 0.131 | 0.100 |
| Ex. 39 | 4-45 | 11-21 | 9.2 | 11.0 | 1500 | 13.6 | 85.2 | 0.132 | 0.100 |
| Ex. 40 | 5-91 | 11-21 | 9.3 | 10.8 | 1500 | 13.9 | 85.0 | 0.132 | 0.100 |
| Ex. 41 | 6-14 | 11-21 | 9.3 | 10.9 | 1500 | 13.8 | 86.2 | 0.131 | 0.100 |
| Ex. 42 | 6-15 | 11-21 | 9.2 | 10.9 | 1500 | 13.8 | 85.7 | 0.131 | 0.100 |
| Ex. 43 | 6-32 | 11-21 | 9.2 | 11.0 | 1500 | 13.6 | 85.4 | 0.131 | 0.100 |
| Ex. 44 | 6-33 | 11-21 | 9.2 | 11.1 | 1500 | 13.5 | 85.9 | 0.131 | 0.100 |
| Ex. 45 | 6-35 | 11-21 | 9.3 | 11.2 | 1500 | 13.4 | 84.7 | 0.131 | 0.100 |

### [Examples 46 to 90] Tandem Organic Electronic Device with Three Stacks Connected

Tandem organic electronic devices were fabricated in the same method as Examples 1 to 45, except that the tandem organic electronic devices were fabricated by connecting three stacks.

### Comparative Examples 4 and 5

Tandem organic electronic devices were fabricated in the same method as Example 46, except that NPB and compound 4-21 of the present disclosure were respectively used as the first hole transport material and the hole transport layer II was not used.

### Comparative Example 6

A tandem organic electronic device was fabricated in the same method as Example 46, except that NPB was used as the first hole transport material and compound 6-30 of the present disclosure was used as the second hole transport material.

TABLE 7

| | 1^{st} | 2^{nd} | Driving | Current | Luminance | Efficiency | | | |
|---|---|---|---|---|---|---|---|---|---|
| | HTM | HTM | voltage | (mA/cm²) | (csd/m²) | (cd/A) | T (95) | CIE (x) | CIE (y) |
| Comp. Ex. 4 | NPB | — | 15.4 | 11.5 | 1500 | 13.0 | 90.1 | 0.126 | 0.100 |
| Comp. Ex. 5 | 4-21 | — | 15.3 | 11.4 | 1500 | 13.2 | 92.8 | 0.126 | 0.100 |
| Comp. Ex. 6 | NPB | 6-30 | 15.1 | 11.1 | 1500 | 13.5 | 96.0 | 0.126 | 0.100 |
| Ex. 46 | 1-137 | 6-30 | 14.5 | 9.6 | 1500 | 15.6 | 112.1 | 0.126 | 0.100 |
| Ex. 47 | 4-21 | 6-30 | 14.5 | 9.7 | 1500 | 15.4 | 112.4 | 0.126 | 0.100 |
| Ex. 48 | 4-45 | 6-30 | 14.4 | 9.8 | 1500 | 15.3 | 113.0 | 0.127 | 0.100 |
| Ex. 49 | 5-91 | 6-30 | 14.5 | 9.7 | 1500 | 15.5 | 112.9 | 0.126 | 0.100 |
| Ex. 50 | 6-14 | 6-30 | 14.5 | 9.7 | 1500 | 15.5 | 114.1 | 0.126 | 0.100 |
| Ex. 51 | 6-15 | 6-30 | 14.1 | 9.7 | 1500 | 15.5 | 113.5 | 0.125 | 0.100 |
| Ex. 52 | 6-32 | 6-30 | 14.4 | 9.9 | 1500 | 15.2 | 113.2 | 0.126 | 0.100 |
| Ex. 53 | 6-33 | 6-30 | 14.4 | 9.9 | 1500 | 15.2 | 113.8 | 0.125 | 0.100 |
| Ex. 54 | 6-35 | 6-30 | 14.5 | 9.9 | 1500 | 15.1 | 112.7 | 0.126 | 0.100 |
| Ex. 55 | 1-137 | 6-31 | 14.4 | 9.8 | 1500 | 15.3 | 110.0 | 0.127 | 0.100 |
| Ex. 56 | 4-21 | 6-31 | 14.4 | 9.9 | 1500 | 15.1 | 110.3 | 0.127 | 0.100 |
| Ex. 57 | 4-45 | 6-31 | 14.3 | 10.0 | 1500 | 15.0 | 111.1 | 0.125 | 0.100 |
| Ex. 58 | 5-91 | 6-31 | 14.4 | 9.8 | 1500 | 15.3 | 110.9 | 0.125 | 0.100 |
| Ex. 59 | 6-14 | 6-31 | 14.4 | 9.9 | 1500 | 15.2 | 112.0 | 0.126 | 0.100 |
| Ex. 60 | 6-15 | 6-31 | 14.3 | 9.9 | 1500 | 15.1 | 111.4 | 0.126 | 0.100 |
| Ex. 61 | 6-32 | 6-31 | 14.3 | 10.1 | 1500 | 14.9 | 111.3 | 0.126 | 0.100 |
| Ex. 62 | 6-33 | 6-31 | 14.3 | 10.1 | 1500 | 14.8 | 111.7 | 0.126 | 0.100 |
| Ex. 63 | 6-35 | 6-31 | 14.4 | 10.1 | 1500 | 14.8 | 110.5 | 0.126 | 0.100 |
| Ex. 64 | 1-137 | 11-14 | 14.1 | 9.3 | 1500 | 16.2 | 118.4 | 0.126 | 0.100 |
| Ex. 65 | 4-21 | 11-14 | 14.1 | 9.4 | 1500 | 16.0 | 118.5 | 0.125 | 0.100 |
| Ex. 66 | 4-45 | 11-14 | 14.0 | 9.4 | 1500 | 15.9 | 118.9 | 0.127 | 0.100 |
| Ex. 67 | 5-91 | 11-14 | 14.1 | 9.3 | 1500 | 16.1 | 118.7 | 0.125 | 0.100 |
| Ex. 68 | 6-14 | 11-14 | 14.1 | 9.3 | 1500 | 16.1 | 119.9 | 0.127 | 0.100 |
| Ex. 69 | 6-15 | 11-14 | 14.0 | 9.4 | 1500 | 16.0 | 119.3 | 0.126 | 0.100 |
| Ex. 70 | 6-32 | 11-14 | 14.0 | 9.4 | 1500 | 15.9 | 119.0 | 0.125 | 0.100 |
| Ex. 71 | 6-33 | 11-14 | 14.0 | 9.5 | 1500 | 15.8 | 119.6 | 0.126 | 0.100 |
| Ex. 72 | 6-35 | 11-14 | 14.1 | 9.6 | 1500 | 15.7 | 118.6 | 0.126 | 0.100 |
| Ex. 73 | 1-137 | 11-20 | 14.2 | 9.1 | 1500 | 16.4 | 116.3 | 0.127 | 0.100 |
| Ex. 74 | 4-21 | 11-20 | 14.2 | 9.3 | 1500 | 16.2 | 116.5 | 0.125 | 0.100 |
| Ex. 75 | 4-45 | 11-20 | 14.1 | 9.3 | 1500 | 16.2 | 117.0 | 0.127 | 0.100 |
| Ex. 76 | 5-91 | 11-20 | 14.2 | 9.1 | 1500 | 16.4 | 116.8 | 0.125 | 0.100 |
| Ex. 77 | 6-14 | 11-20 | 14.2 | 9.2 | 1500 | 16.3 | 118.2 | 0.127 | 0.100 |
| Ex. 78 | 6-15 | 11-20 | 14.1 | 9.3 | 1500 | 16.2 | 117.5 | 0.127 | 0.100 |
| Ex. 79 | 6-32 | 11-20 | 14.1 | 9.3 | 1500 | 16.1 | 117.2 | 0.125 | 0.100 |
| Ex. 80 | 6-33 | 11-20 | 14.1 | 9.3 | 1500 | 16.1 | 117.9 | 0.125 | 0.100 |
| Ex. 81 | 6-35 | 11-20 | 14.2 | 9.4 | 1500 | 16.0 | 116.7 | 0.125 | 0.100 |
| Ex. 82 | 1-137 | 11-21 | 14.3 | 9.4 | 1500 | 15.9 | 114.3 | 0.127 | 0.100 |
| Ex. 83 | 4-21 | 11-21 | 14.3 | 9.6 | 1500 | 15.7 | 114.5 | 0.127 | 0.100 |
| Ex. 84 | 4-45 | 11-21 | 14.2 | 9.6 | 1500 | 15.6 | 115.3 | 0.126 | 0.100 |
| Ex. 85 | 5-91 | 11-21 | 14.3 | 9.4 | 1500 | 15.9 | 115.1 | 0.126 | 0.100 |
| Ex. 86 | 6-14 | 11-21 | 14.3 | 9.5 | 1500 | 15.8 | 116.2 | 0.126 | 0.100 |
| Ex. 87 | 6-15 | 11-21 | 14.2 | 9.6 | 1500 | 15.7 | 115.7 | 0.127 | 0.100 |
| Ex. 88 | 6-32 | 11-21 | 14.2 | 9.6 | 1500 | 15.6 | 115.4 | 0.126 | 0.100 |
| Ex. 89 | 6-33 | 11-21 | 14.2 | 9.7 | 1500 | 15.5 | 116.0 | 0.126 | 0.100 |
| Ex. 90 | 6-35 | 11-21 | 14.3 | 9.7 | 1500 | 15.4 | 114.8 | 0.126 | 0.100 |

### [Examples 91 to 135] Tandem Organic Electronic Devices with Four Stacks Connected

Tandem organic electronic devices were fabricated in the same method as Examples 1 to 45, except that the tandem organic electronic devices were fabricated by connecting four stacks.

### Comparative Examples 7 and 8

Tandem organic electronic devices were fabricated in the same method as Example 91, except that NPB and compound 4-21 of the present disclosure were respectively used as the first hole transport material and the hole transport layer II was not used.

### Comparative Example 9

A tandem organic electronic device was fabricated in the same method as Example 9, except that NPB was used as the first hole transport material and compound 6-30 of the present disclosure was used as the second hole transport material.

TABLE 8

| | 1^{st} | 2^{nd} | Driving | Current | Luminance | Efficiency | | | |
|---|---|---|---|---|---|---|---|---|---|
| | HTM | HTM | voltage | (mA/cm²) | (csd/m²) | (cd/A) | T (95) | CIE (x) | CIE (y) |
| Comp. Ex. 7 | NPB | — | 20.5 | 10.7 | 1500 | 14.0 | 110.0 | 0.125 | 0.100 |
| Comp. Ex. 8 | 4-21 | — | 20.2 | 10.6 | 1500 | 14.2 | 112.6 | 0.124 | 0.100 |
| Comp. Ex. 9 | NPB | 6-30 | 20.2 | 10.3 | 1500 | 14.5 | 117.4 | 0.125 | 0.100 |
| Ex. 91 | 1-137 | 6-30 | 19.5 | 9.0 | 1500 | 16.6 | 130.0 | 0.125 | 0.100 |
| Ex. 92 | 4-21 | 6-30 | 19.5 | 9.1 | 1500 | 16.4 | 130.4 | 0.125 | 0.100 |
| Ex. 93 | 4-45 | 6-30 | 19.4 | 9.2 | 1500 | 16.3 | 131.0 | 0.125 | 0.100 |
| Ex. 94 | 5-91 | 6-30 | 19.5 | 9.1 | 1500 | 16.5 | 130.7 | 0.125 | 0.100 |
| Ex. 95 | 6-14 | 6-30 | 19.5 | 9.1 | 1500 | 16.5 | 132.0 | 0.124 | 0.100 |
| Ex. 96 | 6-15 | 6-30 | 19.4 | 9.1 | 1500 | 16.4 | 131.5 | 0.125 | 0.100 |
| Ex. 97 | 6-32 | 6-30 | 19.4 | 9.2 | 1500 | 16.3 | 131.3 | 0.125 | 0.100 |
| Ex. 98 | 6-33 | 6-30 | 19.4 | 9.3 | 1500 | 16.2 | 131.8 | 0.124 | 0.100 |
| Ex. 99 | 6-35 | 6-30 | 19.5 | 9.3 | 1500 | 16.1 | 130.1 | 0.125 | 0.100 |
| Ex. 100 | 1-137 | 6-31 | 19.4 | 9.2 | 1500 | 16.3 | 132.2 | 0.125 | 0.100 |
| Ex. 101 | 4-21 | 6-31 | 19.4 | 9.3 | 1500 | 16.1 | 132.5 | 0.125 | 0.100 |
| Ex. 102 | 4-45 | 6-31 | 19.3 | 9.4 | 1500 | 16.0 | 133.0 | 0.125 | 0.100 |
| Ex. 103 | 5-91 | 6-31 | 19.4 | 9.2 | 1500 | 16.3 | 132.9 | 0.125 | 0.100 |
| Ex. 104 | 6-14 | 6-31 | 19.4 | 9.3 | 1500 | 16.2 | 134.1 | 0.124 | 0.100 |
| Ex. 105 | 6-15 | 6-31 | 19.3 | 9.3 | 1500 | 16.1 | 133.6 | 0.124 | 0.100 |
| Ex. 106 | 6-32 | 6-31 | 19.3 | 9.4 | 1500 | 15.9 | 133.3 | 0.124 | 0.100 |
| Ex. 107 | 6-33 | 6-31 | 19.3 | 9.5 | 1500 | 15.8 | 133.8 | 0.125 | 0.100 |
| Ex. 108 | 6-35 | 6-31 | 19.4 | 9.5 | 1500 | 15.8 | 132.7 | 0.125 | 0.100 |
| Ex. 109 | 1-137 | 11-14 | 19.1 | 8.7 | 1500 | 17.2 | 138.4 | 0.125 | 0.100 |
| Ex. 110 | 4-21 | 11-14 | 19.1 | 8.9 | 1500 | 16.9 | 138.5 | 0.126 | 0.100 |
| Ex. 111 | 4-45 | 11-14 | 19.0 | 8.9 | 1500 | 16.9 | 139.0 | 0.125 | 0.100 |
| Ex. 112 | 5-91 | 11-14 | 19.1 | 8.8 | 1500 | 17.1 | 138.8 | 0.126 | 0.100 |
| Ex. 113 | 6-14 | 11-14 | 19.1 | 8.8 | 1500 | 17.1 | 140.0 | 0.125 | 0.100 |
| Ex. 114 | 6-15 | 11-14 | 19.0 | 8.8 | 1500 | 17.0 | 139.4 | 0.125 | 0.100 |
| Ex. 115 | 6-32 | 11-14 | 19.0 | 8.9 | 1500 | 16.8 | 139.2 | 0.125 | 0.100 |
| Ex. 116 | 6-33 | 11-14 | 19.0 | 9.0 | 1500 | 16.7 | 139.7 | 0.124 | 0.100 |
| Ex. 117 | 6-35 | 11-14 | 19.1 | 9.0 | 1500 | 16.7 | 138.6 | 0.125 | 0.100 |
| Ex. 118 | 1-137 | 11-20 | 19.2 | 8.6 | 1500 | 17.4 | 136.3 | 0.126 | 0.100 |
| Ex. 119 | 4-21 | 11-20 | 19.2 | 8.7 | 1500 | 17.2 | 136.6 | 0.125 | 0.100 |
| Ex. 120 | 4-45 | 11-20 | 19.1 | 8.7 | 1500 | 17.2 | 137.3 | 0.125 | 0.100 |
| Ex. 121 | 5-91 | 11-20 | 19.2 | 8.6 | 1500 | 17.4 | 137.1 | 0.125 | 0.100 |
| Ex. 122 | 6-14 | 11-20 | 19.2 | 8.6 | 1500 | 17.4 | 138.3 | 0.125 | 0.100 |
| Ex. 123 | 6-15 | 11-20 | 19.1 | 8.7 | 1500 | 17.3 | 137.9 | 0.125 | 0.100 |
| Ex. 124 | 6-32 | 11-20 | 19.1 | 8.8 | 1500 | 17.1 | 137.6 | 0.125 | 0.100 |
| Ex. 125 | 6-33 | 11-20 | 19.1 | 8.8 | 1500 | 17.1 | 138.1 | 0.124 | 0.100 |
| Ex. 126 | 6-35 | 11-20 | 19.2 | 8.8 | 1500 | 17.0 | 136.8 | 0.124 | 0.100 |
| Ex. 127 | 1-137 | 11-21 | 19.3 | 8.9 | 1500 | 16.9 | 134.2 | 0.124 | 0.100 |
| Ex. 128 | 4-21 | 11-21 | 19.3 | 9.0 | 1500 | 16.7 | 134.3 | 0.125 | 0.100 |
| Ex. 129 | 4-45 | 11-21 | 19.2 | 9.0 | 1500 | 16.7 | 135.1 | 0.124 | 0.100 |
| Ex. 130 | 5-91 | 11-21 | 19.3 | 8.9 | 1500 | 16.9 | 134.8 | 0.125 | 0.100 |
| Ex. 131 | 6-14 | 11-21 | 19.3 | 8.9 | 1500 | 16.8 | 136.2 | 0.124 | 0.100 |
| Ex. 132 | 6-15 | 11-21 | 19.2 | 8.9 | 1500 | 16.8 | 135.7 | 0.126 | 0.100 |
| Ex. 133 | 6-32 | 11-21 | 19.2 | 9.1 | 1500 | 16.6 | 135.4 | 0.124 | 0.100 |
| Ex. 134 | 6-33 | 11-21 | 19.2 | 9.1 | 1500 | 16.5 | 135.9 | 0.125 | 0.100 |
| Ex. 135 | 6-35 | 11-21 | 19.3 | 9.1 | 1500 | 16.4 | 134.6 | 0.126 | 0.100 |

As seen from the result of Tables 6 to 8, it can be appreciated that, when the hole transport region was formed using compound I and compound II of the present disclosure (Examples 1 to 135), the electrical characteristics of the devices were improved than either when NPB or compound 4-21 of the present disclosure, i.e., the first compound represented by Formula C or Formula D and including a radical of a compound represented by Formula A, was used only for the first hole transport layer (Comparative Examples 1, 2 4, 5, 7, and 8) or when NPB was used for the first hole transport material and compound 6-30 of the present disclosure represented by Formula C or Formula D was used for the second hole transport material (Comparative Examples 3, 6, and 9).

Describing in detail, the device characteristics of Comparative Examples 2, 5, and 8, in which compound 4-21 of the present disclosure was used, were more improved than those of Comparative Examples 1, 4, and 7, in which NPB was used for the first hole transport material, and the device characteristics of Comparative Examples 3, 6, and 9, in which NPB was used for the first hole transport material and compound 6-30 of the present disclosure was used for the second hole transport material, were more improved than those of Comparative Examples 1, 2, 4, 5, 7, and 8, in which only the first hole transport material was used. In addition, it can be appreciated that, in Examples 1 to 135, in which the first compound represented by Formula C or Formula D and including the radical of the compound represented by Formula A was used for the first hole transport material and the second compound represented by Formula C or Formula D and including radical of the compound represented by Formula A or Formula B was used for the second hole transport material, the device characteristics, such as the driving voltage, efficiency, and lifespan, were more improved than those of Comparative Examples 1 to 9.

It is considered that the use of the compounds of the present disclosure for the first hole transport material and the second hole transport material caused an appropriate number of holes in the emission layer to efficiently move so as to balance holes and electrons in the emission layer and prevent degradations in the interface of the emission layer, thereby increasing the efficiency and lifespan.

In the meantime, referring to Examples 1 to 45, Examples 46 to 90, and Examples 91 to 135 according to the present disclosure, it can be appreciated that the efficiency and lifespan among the device characteristics were improved with increases in the number of the stacks connected. Specifically, in the devices according to Examples 46 to 90, in which three stacks were connected, the driving voltage was increased but the efficiency and lifespan were improved compared to those of Examples 1 to 45, in which two stacks were connected. In addition, in Examples 91 to 135, in which four stacks were connected, the driving voltage was increased but the efficiency and lifespan were improved compared to those of Examples 46 to 90. It is considered that the efficiency and lifespan were improved proportionally to increases in the number of the stacks, due to the multiphoton emission structure in which excitons are generated to emit light energy in each of the stacks.

In addition, referring to Examples 1 to 45, Examples 46 to 90, and Examples 91 to 135 according to the present disclosure, it can be seen that the values of the color coordinates (CIE x) gradually decreased with increases in the number of the stacks connected. It is considered that the color purity was improved as the full width at half maximum (FWHM) of an emission wavelength was reduced with increases in the number of the stacks.

The final product represented by Formula 1 according to the present disclosure may be manufactured by, but is not limited to, reaction as represented by the following Reaction Formula 18.

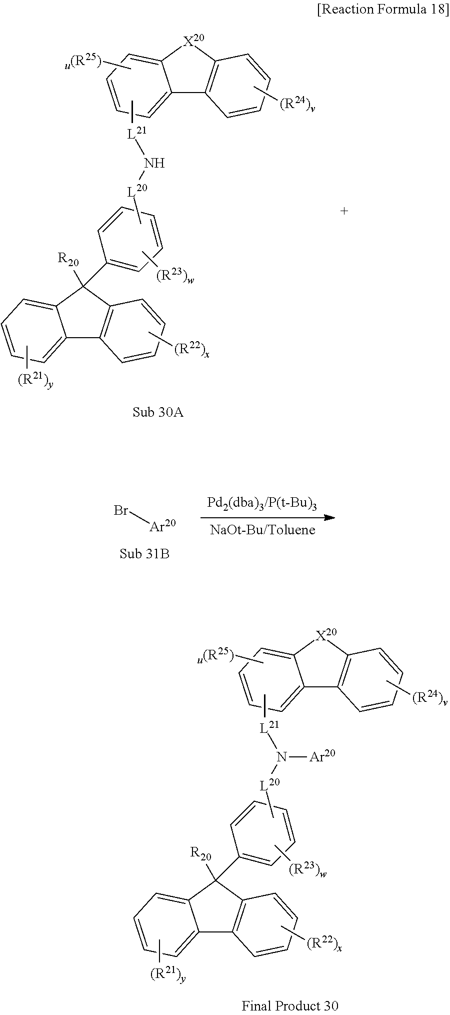

Synthesis of Sub 30A

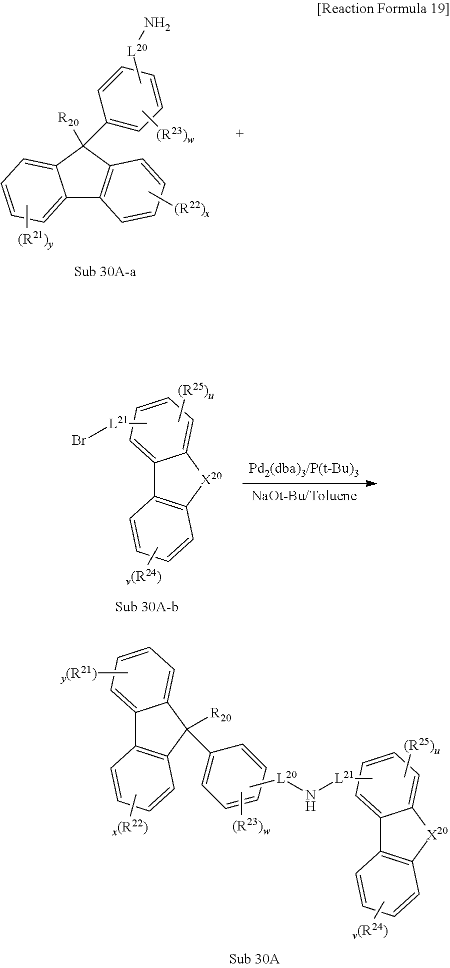

1. Example of Synthesis of Sub 30A-1

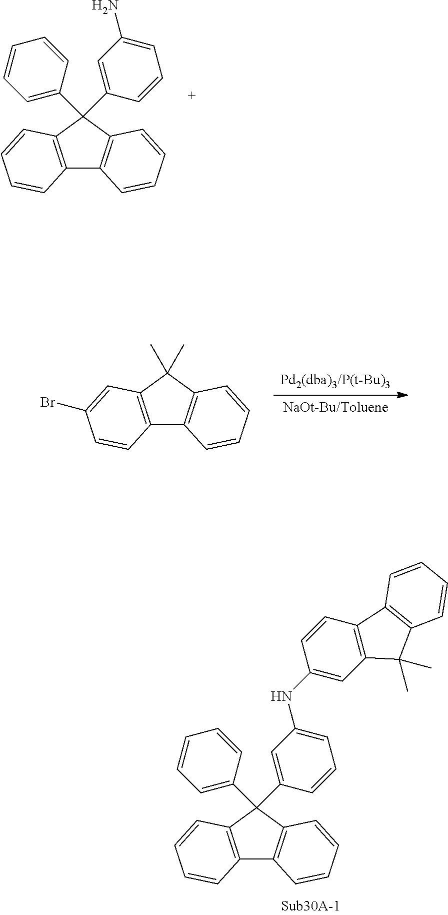 [SMILES: Nc1cccc(C2(c3ccccc3)c3ccccc3-c3ccccc32)c1 ; CC1(C)c2ccccc2-c2ccc(Br)cc21 ; "NaOt-Bu/Toluene" ; "Pd2(dba)3/P(t-Bu)3" ; CC1(C)c2ccccc2-c2ccc(Nc3cccc(C4(c5ccccc5)c5ccccc5-c5ccccc54)c3)cc21]

3-(9-phenyl-9H-fluoren-9-yl) aniline (6.7 g, 20 mmol), 2-bromo-9,9-dimethyl-9H-fluorene (5.7 g, 21.1 mmol), Pd₂(dba)₃ (0.5 g, 0.6 mmol), P(t-Bu)₃ (0.3 g, 1.6 mmol), NaOt-Bu (5.7 g, 60.2 mmol), and toluene (206 ml) were added to a round bottom flask, followed by stirring at 100° C. When the reaction is completed, extraction was performed with ether and water, and an organic layer was dried with MgSO₄ and concentrated. Afterwards, a resultant compound was subjected to silica gel column chromatography and recrystallization, thereby creating Sub 30A-1 8.87 g (yield: 84%).

2. Illustration of Synthesis of Sub 30A-66

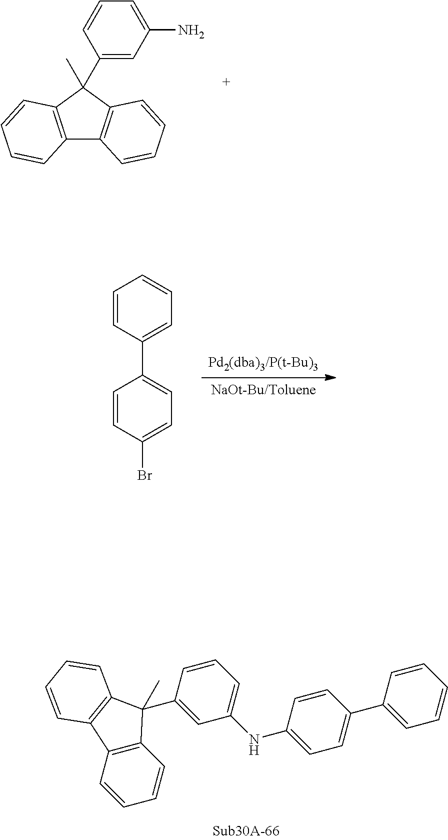 [SMILES: CC1(c2cccc(N)c2)c2ccccc2-c2ccccc21 ; Brc1ccc(-c2ccccc2)cc1 ; "NaOt-Bu/Toluene" ; "Pd2(dba)3/P(t-Bu)3" ; CC1(c2cccc(Nc3ccc(-c4ccccc4)cc3)c2)c2ccccc2-c2ccccc21]

After 3-(9-methyl-9H-fluoren-9-yl)aniline (8.0 g, 29.4 mmol), 4-bromo-1,1′-biphenyl (7.2 g, 30.9 mmol), Pd₂(dba)₃ (0.8 g, 0.8 mmol), P(t-Bu)₃ (0.4 g, 2.3 mmol), NaOt-Bu (8.5 g, 88.4 mmol), and toluene (302 ml) were added to a round bottom flask, and then subjected to the same experimental method as in Sub 30A-1, thereby creating Sub 30A-66 9.99 g (yield: 80%).

3. Illustration of Synthesis of Sub 30A-69

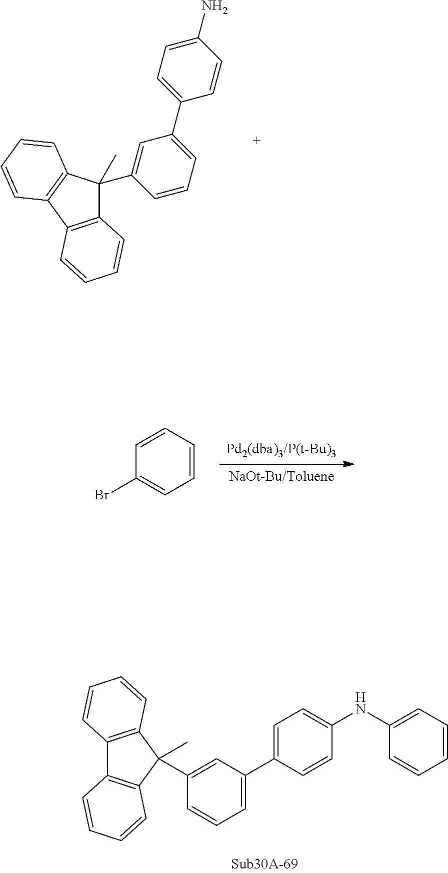 [SMILES: CC1(c2cccc(-c3ccc(N)cc3)c2)c2ccccc2-c2ccccc21 ; Brc1ccccc1 ; "NaOt-Bu/Toluene" ; "Pd2(dba)3/P(t-Bu)3" ; CC1(c2cccc(-c3ccc(Nc4ccccc4)cc3)c2)c2ccccc2-c2ccccc21]

3′-(9-methyl-9H-fluoren-9-yl)-[1,1′-biphenyl]-4-amine (7.6 g, 21.8 mmol), bromobenzene (3.6 g, 22.9 mmol), Pd₂(dba)₃ (0.6 g, 0.6 mmol), P(t-Bu)₃ (0.3 g, 1.7 mmol), NaOt-Bu (6.3 g, 65.6 mmol), and toluene (224 ml) were added to a round bottom flask, and then subjected to the same experimental method as in Sub 30A-1, thereby creating Sub 30A-69 6.86 g (yield: 74%).

The compounds belonging to Sub 30A may be, but are not limited to, the following compounds, and Table 9 illustrates field desorption-mass spectrometry (FD-MS) values of the compounds belonging to Sub 30A.

TABLE 9

| | |
|---|---|
| 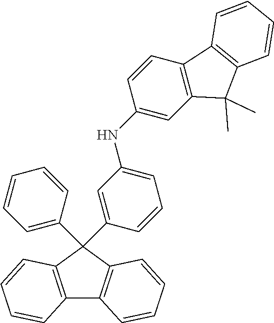 [SMILES: CC1(C)c2ccccc2-c2ccc(Nc3cccc(C4(c5ccccc5)c5ccccc5-c5ccccc54)c3)cc21] | Sub 30A-1 |
| 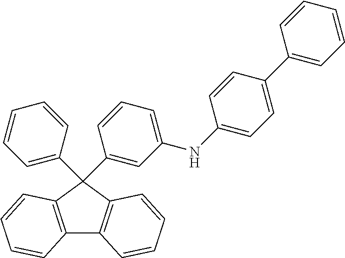 [SMILES: c1ccc(-c2ccc(Nc3cccc(C4(c5ccccc5)c5ccccc5-c5ccccc54)c3)cc2)cc1] | Sub 30A-2 |
| 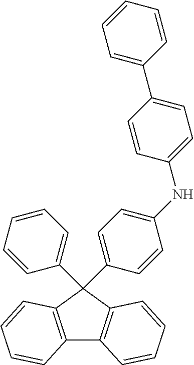 [SMILES: c1ccc(-c2ccc(Nc3ccc(C4(c5ccccc5)c5ccccc5-c5ccccc54)cc3)cc2)cc1] | Sub 30A-3 |
| 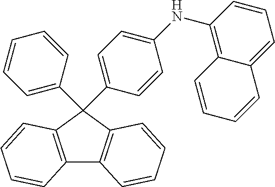 [SMILES: c1ccc(C2(c3ccc(Nc4cccc5ccccc45)cc3)c3ccccc3-c3ccccc32)cc1] | Sub 30A-4 |
| 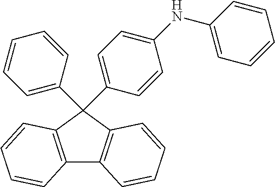 [SMILES: c1ccc(Nc2ccc(C3(c4ccccc4)c4ccccc4-c4ccccc43)cc2)cc1] | Sub 30A-5 |
| 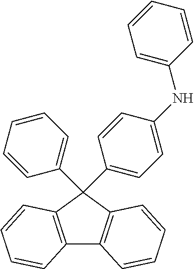 [SMILES: c1ccc(Nc2ccc(C3(c4ccccc4)c4ccccc4-c4ccccc43)cc2)cc1] | Sub 30A-6 |
| 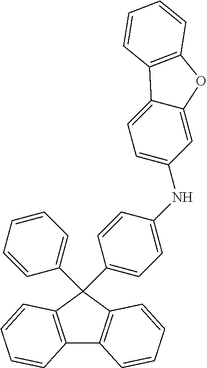 [SMILES: c1ccc(C2(c3ccc(Nc4ccc5c(c4)oc4ccccc45)cc3)c3ccccc3-c3ccccc32)cc1] | Sub 30A-7 |
| 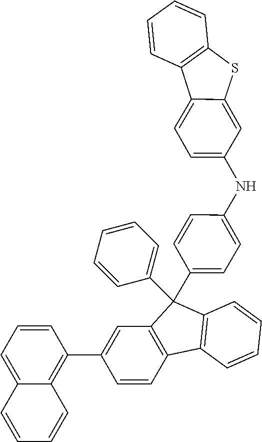 [SMILES: c1ccc(C2(c3ccc(Nc4ccc5c(c4)sc4ccccc45)cc3)c3ccccc3-c3ccc(-c4cccc5ccccc45)cc32)cc1] | Sub 30A-8 |
| 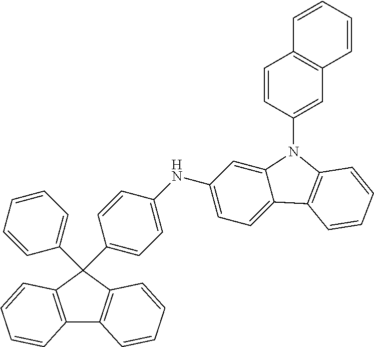 [SMILES: c1ccc(C2(c3ccc(Nc4ccc5c6ccccc6n(-c6ccc7ccccc7c6)c5c4)cc3)c3ccccc3-c3ccccc32)cc1] | Sub 30A-9 |
| 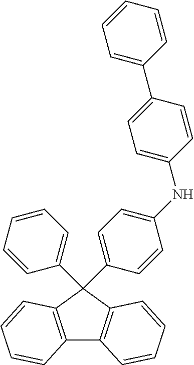 [SMILES: c1ccc(-c2ccc(Nc3ccc(C4(c5ccccc5)c5ccccc5-c5ccccc54)cc3)cc2)cc1] | Sub 30A-10 |
| 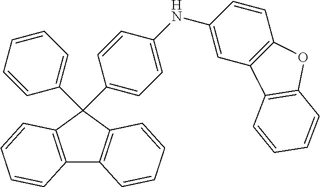 [SMILES: c1ccc(C2(c3ccc(Nc4ccc5oc6ccccc6c5c4)cc3)c3ccccc3-c3ccccc32)cc1] | Sub 30A-11 |
| 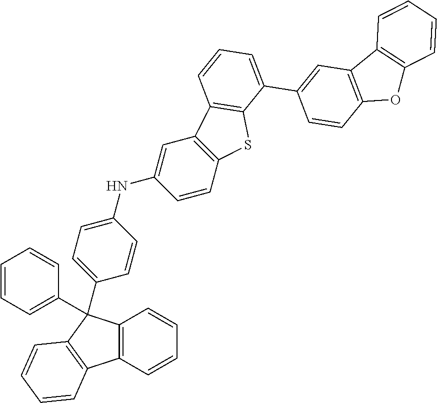 [SMILES: c1ccc(C2(c3ccc(Nc4ccc5sc6c(-c7ccc8oc9ccccc9c8c7)cccc6c5c4)cc3)c3ccccc3-c3ccccc32)cc1] | Sub 30A-12 |
| 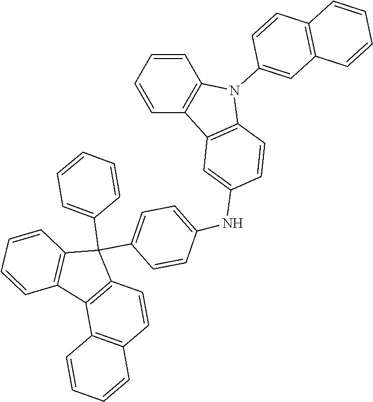 [SMILES: c1ccc(C2(c3ccc(Nc4ccc5c(c4)c4ccccc4n5-c4ccc5ccccc5c4)cc3)c3ccccc3-c3c2ccc2ccccc32)cc1] | Sub 30A-13 |
| 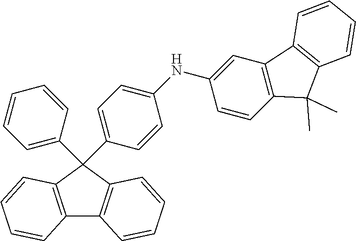 [SMILES: CC1(C)c2ccccc2-c2cc(Nc3ccc(C4(c5ccccc5)c5ccccc5-c5ccccc54)cc3)ccc21] | Sub 30A-14 |
| 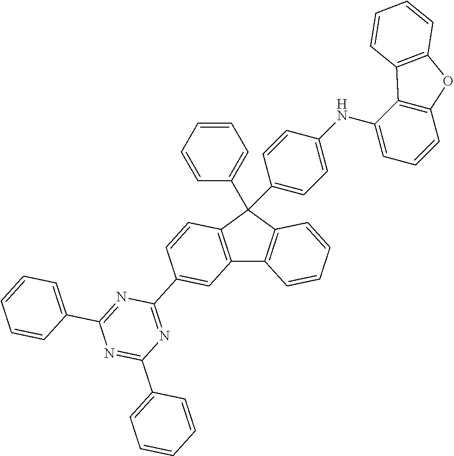 [SMILES: c1ccc(-c2nc(-c3ccccc3)nc(-c3ccc4c(c3)-c3ccccc3C4(c3ccccc3)c3ccc(Nc4cccc5oc6ccccc6c45)cc3)n2)cc1] | Sub 30A-15 |
| 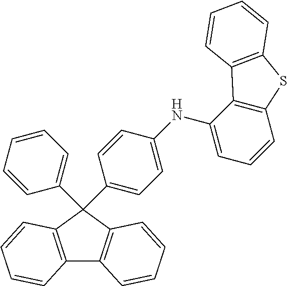 [SMILES: c1ccc(C2(c3ccc(Nc4cccc5sc6ccccc6c45)cc3)c3ccccc3-c3ccccc32)cc1] | Sub 30A-16 |
| 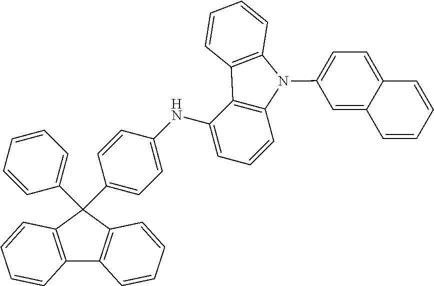 [SMILES: c1ccc(C2(c3ccc(Nc4cccc5c4c4ccccc4n5-c4ccc5ccccc5c4)cc3)c3ccccc3-c3ccccc32)cc1] | Sub 30A-17 |
| 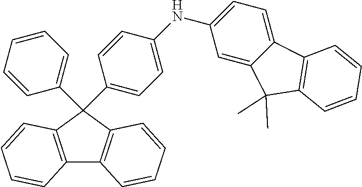 [SMILES: CC1(C)c2ccccc2-c2ccc(Nc3ccc(C4(c5ccccc5)c5ccccc5-c5ccccc54)cc3)cc21] | Sub 30A-18 |
| 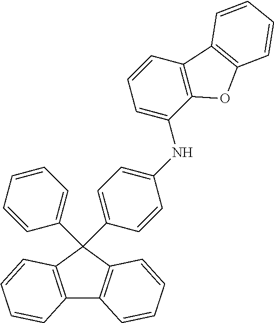 [SMILES: c1ccc(C2(c3ccc(Nc4cccc5c4oc4ccccc45)cc3)c3ccccc3-c3ccccc32)cc1] | Sub 30A-19 |
| 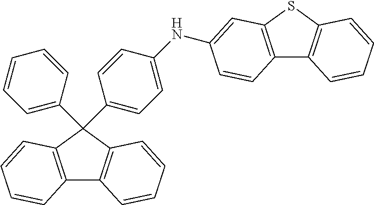 [SMILES: c1ccc(C2(c3ccc(Nc4ccc5c(c4)sc4ccccc45)cc3)c3ccccc3-c3ccccc32)cc1] | Sub 30A-20 |
| 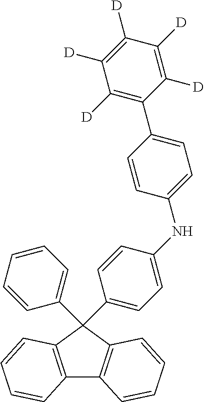 [SMILES: [2H]c1c([2H])c([2H])c(-c2ccc(Nc3ccc(C4(c5ccccc5)c5ccccc5-c5ccccc54)cc3)cc2)c([2H])c1[2H]] | Sub 30A-21 |
| 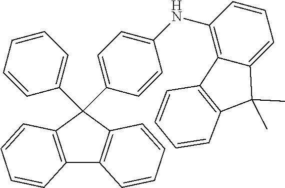 [SMILES: CC1(C)c2ccccc2-c2c(Nc3ccc(C4(c5ccccc5)c5ccccc5-c5ccccc54)cc3)cccc21] | Sub 30A-22 |
| 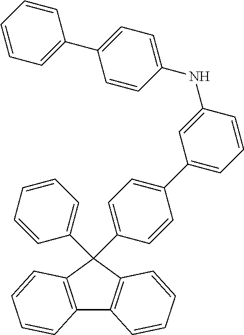 [SMILES: c1ccc(-c2ccc(Nc3cccc(-c4ccc(C5(c6ccccc6)c6ccccc6-c6ccccc65)cc4)c3)cc2)cc1] | Sub 30A-23 |
| 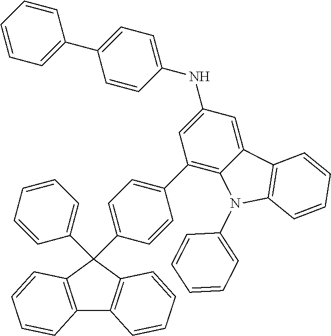 [SMILES: c1ccc(-c2ccc(Nc3cc(-c4ccc(C5(c6ccccc6)c6ccccc6-c6ccccc65)cc4)c4c(c3)c3ccccc3n4-c3ccccc3)cc2)cc1] | Sub 30A-24 |
| 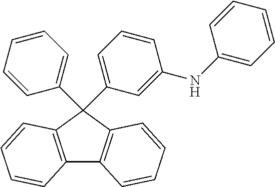 [SMILES: c1ccc(Nc2cccc(C3(c4ccccc4)c4ccccc4-c4ccccc43)c2)cc1] | Sub 30A-25 |
| 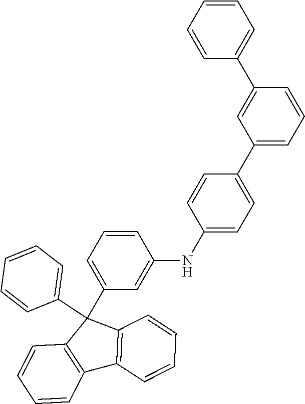 [SMILES: c1ccc(-c2cccc(-c3ccc(Nc4cccc(C5(c6ccccc6)c6ccccc6-c6ccccc65)c4)cc3)c2)cc1] | Sub 30A-26 |
| 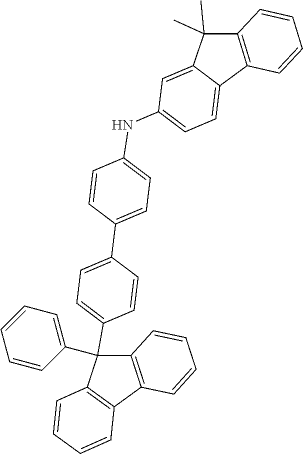 [SMILES: CC1(C)c2ccccc2-c2ccc(Nc3ccc(-c4ccc(C5(c6ccccc6)c6ccccc6-c6ccccc65)cc4)cc3)cc21] | Sub 30A-27 |
| 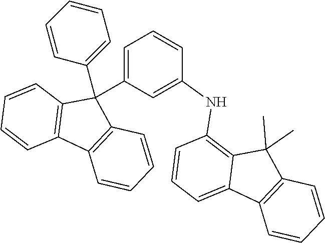 [SMILES: CC1(C)c2ccccc2-c2cccc(Nc3cccc(C4(c5ccccc5)c5ccccc5-c5ccccc54)c3)c21] | Sub 30A-28 |
| 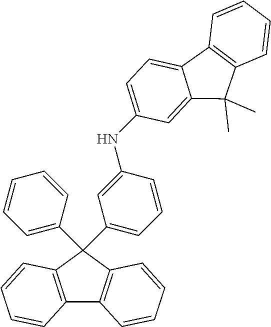 [SMILES: CC1(C)c2ccccc2-c2ccc(Nc3cccc(C4(c5ccccc5)c5ccccc5-c5ccccc54)c3)cc21] | Sub 30A-29 |
| 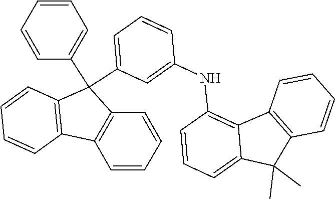 [SMILES: CC1(C)c2ccccc2-c2c(Nc3cccc(C4(c5ccccc5)c5ccccc5-c5ccccc54)c3)cccc21] | Sub 30A-30 |
| 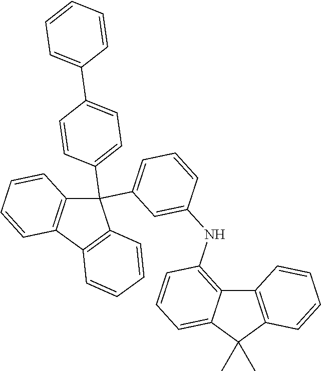 [SMILES: CC1(C)c2ccccc2-c2c(Nc3cccc(C4(c5ccc(-c6ccccc6)cc5)c5ccccc5-c5ccccc54)c3)cccc21] | Sub 30A-31 |
| 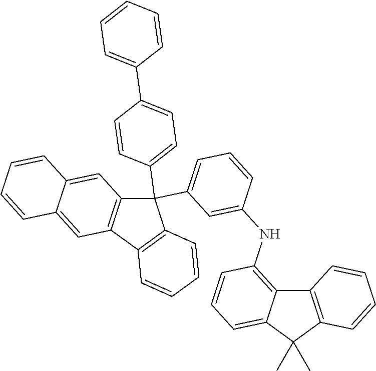 [SMILES: CC1(C)c2ccccc2-c2c(Nc3cccc(C4(c5ccc(-c6ccccc6)cc5)c5ccccc5-c5cc6ccccc6cc54)c3)cccc21] | Sub 30A-32 |
| 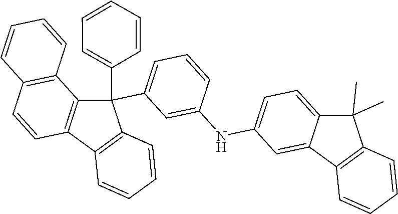 [SMILES: CC1(C)c2ccccc2-c2cc(Nc3cccc(C4(c5ccccc5)c5ccccc5-c5ccc6ccccc6c54)c3)ccc21] | Sub 30A-33 |
| 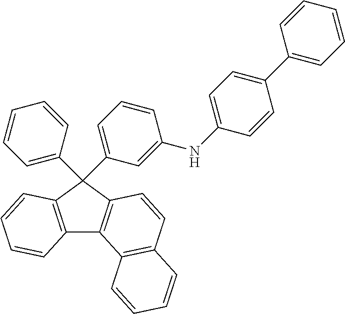 [SMILES: c1ccc(-c2ccc(Nc3cccc(C4(c5ccccc5)c5ccccc5-c5c4ccc4ccccc54)c3)cc2)cc1] | Sub 30A-34 |
| 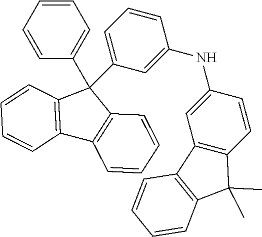 [SMILES: CC1(C)c2ccccc2-c2cc(Nc3cccc(C4(c5ccccc5)c5ccccc5-c5ccccc54)c3)ccc21] | Sub 30A-35 |
| 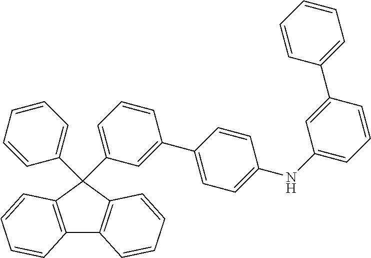 [SMILES: c1ccc(-c2cccc(Nc3ccc(-c4cccc(C5(c6ccccc6)c6ccccc6-c6ccccc65)c4)cc3)c2)cc1] | Sub 30A-36 |
| 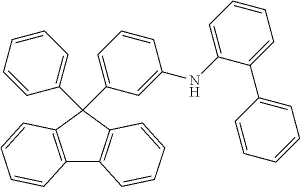 [SMILES: c1ccc(-c2ccccc2Nc2cccc(C3(c4ccccc4)c4ccccc4-c4ccccc43)c2)cc1] | Sub 30A-37 |
| 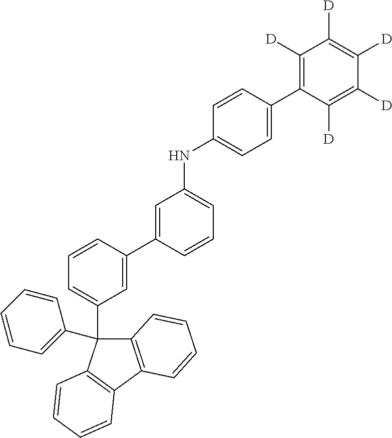 [SMILES: [2H]c1c([2H])c([2H])c(-c2ccc(Nc3cccc(-c4cccc(C5(c6ccccc6)c6ccccc6-c6ccccc65)c4)c3)cc2)c([2H])c1[2H]] | Sub 30A-38 |
| 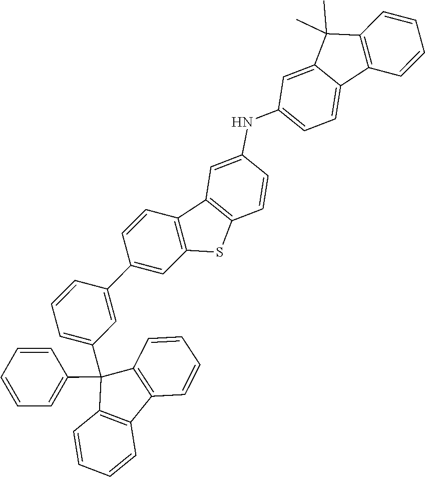 [SMILES: CC1(C)c2ccccc2-c2ccc(Nc3ccc4sc5cc(-c6cccc(C7(c8ccccc8)c8ccccc8-c8ccccc87)c6)ccc5c4c3)cc21] | Sub 30A-39 |
| 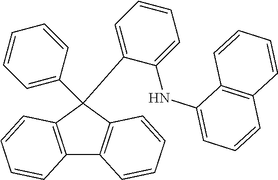 [SMILES: c1ccc(C2(c3ccccc3Nc3cccc4ccccc34)c3ccccc3-c3ccccc32)cc1] | Sub 30A-40 |
| 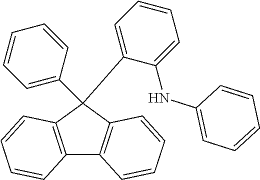 [SMILES: c1ccc(Nc2ccccc2C2(c3ccccc3)c3ccccc3-c3ccccc32)cc1] | Sub 30A-41 |
| 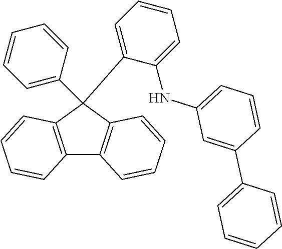 [SMILES: c1ccc(-c2cccc(Nc3ccccc3C3(c4ccccc4)c4ccccc4-c4ccccc43)c2)cc1] | Sub 30A-42 |
| 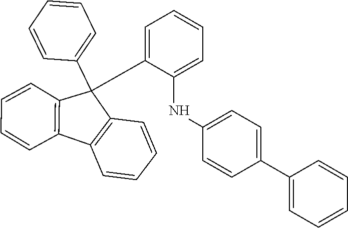 [SMILES: c1ccc(-c2ccc(Nc3ccccc3C3(c4ccccc4)c4ccccc4-c4ccccc43)cc2)cc1] | Sub 30A-43 |
| 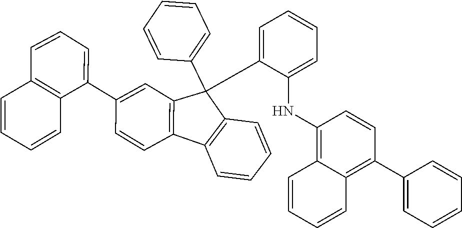 [SMILES: c1ccc(-c2ccc(Nc3ccccc3C3(c4ccccc4)c4ccccc4-c4ccc(-c5cccc6ccccc56)cc43)c3ccccc23)cc1] | Sub 30A-44 |
| 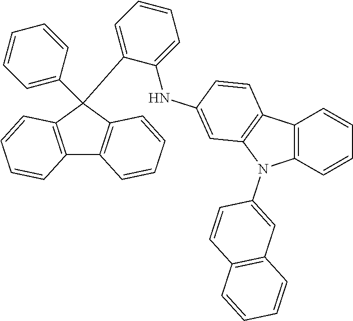 [SMILES: c1ccc(C2(c3ccccc3Nc3ccc4c5ccccc5n(-c5ccc6ccccc6c5)c4c3)c3ccccc3-c3ccccc32)cc1] | Sub 30A-45 |
| 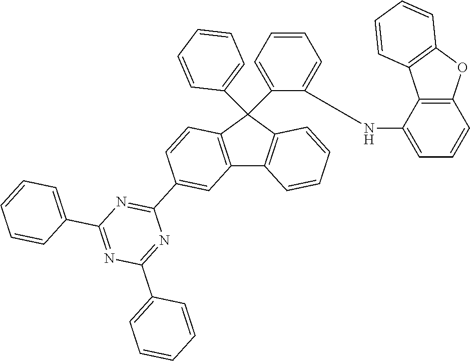 [SMILES: c1ccc(-c2nc(-c3ccccc3)nc(-c3ccc4c(c3)-c3ccccc3C4(c3ccccc3)c3ccccc3Nc3cccc4oc5ccccc5c34)n2)cc1] | Sub 30A-46 |
| 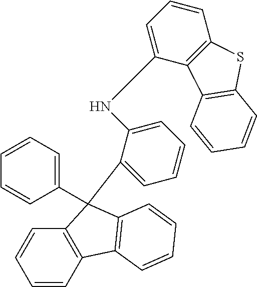 [SMILES: c1ccc(C2(c3ccccc3Nc3cccc4sc5ccccc5c34)c3ccccc3-c3ccccc32)cc1] | Sub 30A-47 |
| 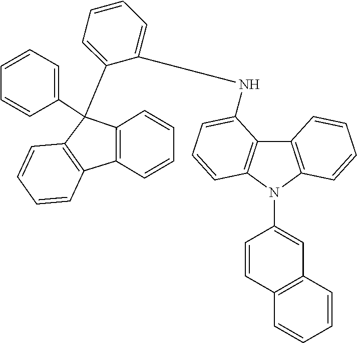 [SMILES: c1ccc(C2(c3ccccc3Nc3cccc4c3c3ccccc3n4-c3ccc4ccccc4c3)c3ccccc3-c3ccccc32)cc1] | Sub 30A-48 |
| 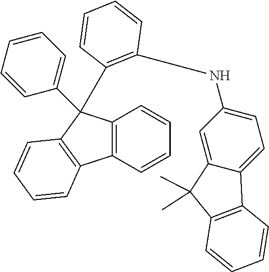 [SMILES: CC1(C)c2ccccc2-c2ccc(Nc3ccccc3C3(c4ccccc4)c4ccccc4-c4ccccc43)cc21] | Sub 30A-49 |
| 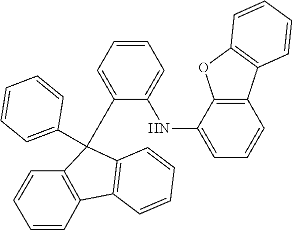 [SMILES: c1ccc(C2(c3ccccc3Nc3cccc4c3oc3ccccc34)c3ccccc3-c3ccccc32)cc1] | Sub 30A-50 |
| 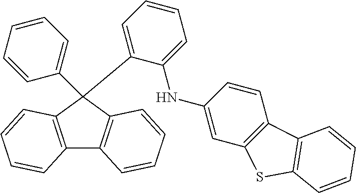 [SMILES: c1ccc(C2(c3ccccc3Nc3ccc4c(c3)sc3ccccc34)c3ccccc3-c3ccccc32)cc1] | Sub 30A-51 |
| 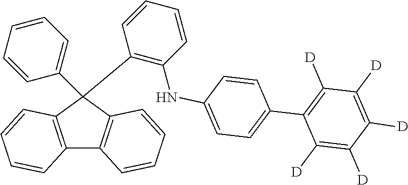 [SMILES: [2H]c1c([2H])c([2H])c(-c2ccc(Nc3ccccc3C3(c4ccccc4)c4ccccc4-c4ccccc43)cc2)c([2H])c1[2H]] | Sub 30A-52 |
| 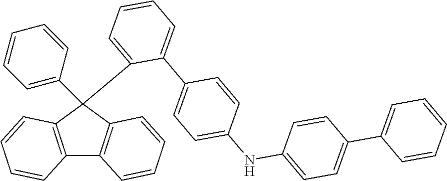 [SMILES: c1ccc(-c2ccc(Nc3ccc(-c4ccccc4C4(c5ccccc5)c5ccccc5-c5ccccc54)cc3)cc2)cc1] | Sub 30A-53 |
| 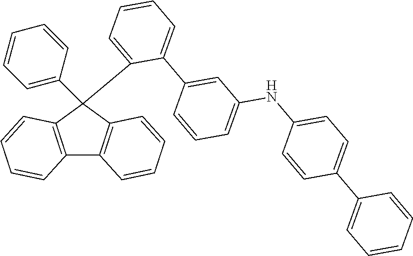 [SMILES: c1ccc(-c2ccc(Nc3cccc(-c4ccccc4C4(c5ccccc5)c5ccccc5-c5ccccc54)c3)cc2)cc1] | Sub 30A-54 |
| 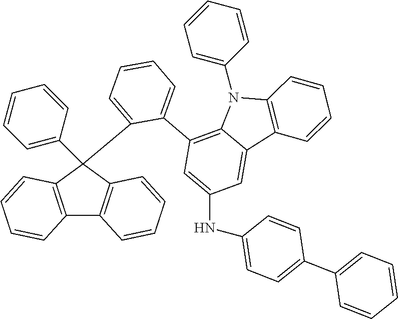 [SMILES: c1ccc(-c2ccc(Nc3cc(-c4ccccc4C4(c5ccccc5)c5ccccc5-c5ccccc54)c4c(c3)c3ccccc3n4-c3ccccc3)cc2)cc1] | Sub 30A-55 |
| 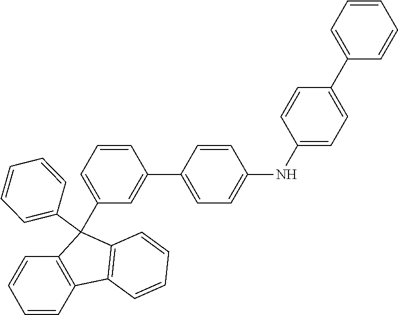 [SMILES: c1ccc(-c2ccc(Nc3ccc(-c4cccc(C5(c6ccccc6)c6ccccc6-c6ccccc65)c4)cc3)cc2)cc1] | Sub 30A-56 |
| 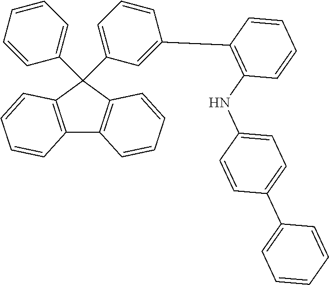 [SMILES: c1ccc(-c2ccc(Nc3ccccc3-c3cccc(C4(c5ccccc5)c5ccccc5-c5ccccc54)c3)cc2)cc1] | Sub 30A-57 |
| 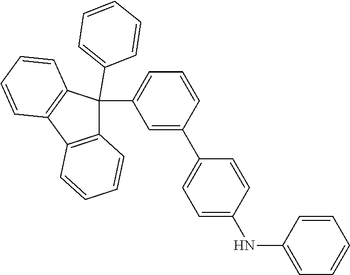 [SMILES: c1ccc(Nc2ccc(-c3cccc(C4(c5ccccc5)c5ccccc5-c5ccccc54)c3)cc2)cc1] | Sub 30A-58 |
| 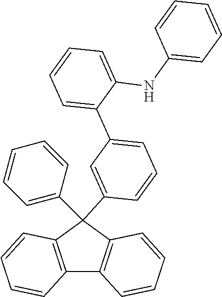 [SMILES: c1ccc(Nc2ccccc2-c2cccc(C3(c4ccccc4)c4ccccc4-c4ccccc43)c2)cc1] | Sub 30A-59 |
| 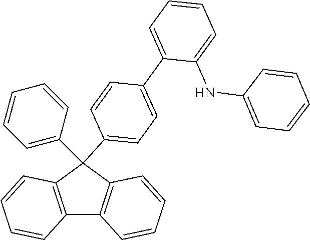 [SMILES: c1ccc(Nc2ccccc2-c2ccc(C3(c4ccccc4)c4ccccc4-c4ccccc43)cc2)cc1] | Sub 30A-60 |
| 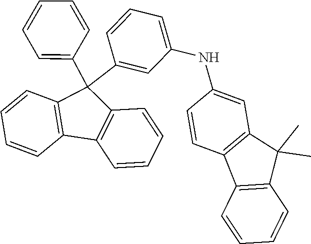 [SMILES: CC1(C)c2ccccc2-c2ccc(Nc3cccc(C4(c5ccccc5)c5ccccc5-c5ccccc54)c3)cc21] | Sub 30A-61 |
| 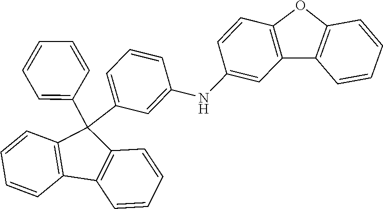 [SMILES: c1ccc(C2(c3cccc(Nc4ccc5oc6ccccc6c5c4)c3)c3ccccc3-c3ccccc32)cc1] | Sub 30A-62 |
| 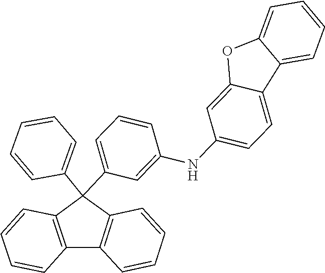 [SMILES: c1ccc(C2(c3cccc(Nc4ccc5c(c4)oc4ccccc45)c3)c3ccccc3-c3ccccc32)cc1] | Sub 30A-63 |
| 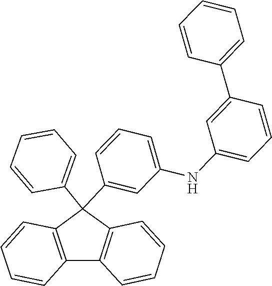 [SMILES: c1ccc(-c2cccc(Nc3cccc(C4(c5ccccc5)c5ccccc5-c5ccccc54)c3)c2)cc1] | Sub 30A-64 |
| 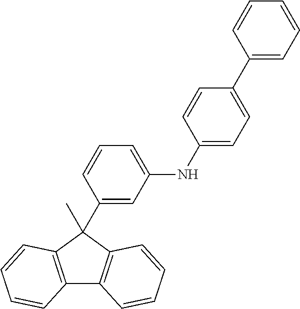 [SMILES: CC1(c2cccc(Nc3ccc(-c4ccccc4)cc3)c2)c2ccccc2-c2ccccc21] | Sub 30A-65 |
| 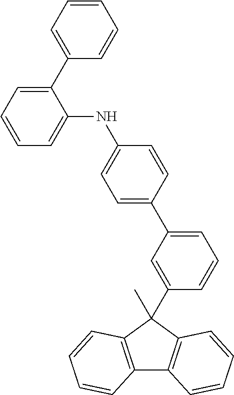 [SMILES: CC1(c2cccc(-c3ccc(Nc4ccccc4-c4ccccc4)cc3)c2)c2ccccc2-c2ccccc21] | Sub 30A-66 |
| 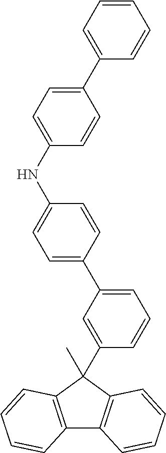 [SMILES: CC1(c2cccc(-c3ccc(Nc4ccc(-c5ccccc5)cc4)cc3)c2)c2ccccc2-c2ccccc21] | Sub 30A-67 |
| 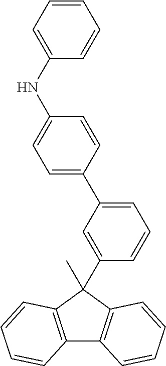 [SMILES: CC1(c2cccc(-c3ccc(Nc4ccccc4)cc3)c2)c2ccccc2-c2ccccc21] | Sub 30A-68 |
| 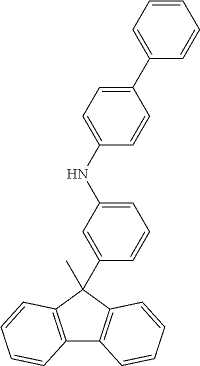 [SMILES: CC1(c2cccc(Nc3ccc(-c4ccccc4)cc3)c2)c2ccccc2-c2ccccc21] | Sub 30A-69 |
| 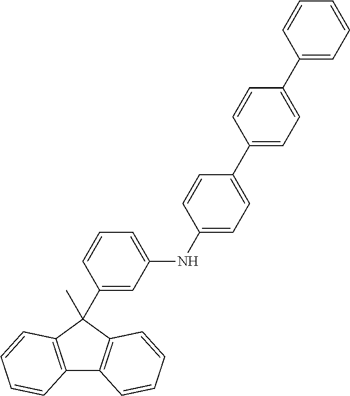 [SMILES: CC1(c2cccc(Nc3ccc(-c4ccc(-c5ccccc5)cc4)cc3)c2)c2ccccc2-c2ccccc21] | Sub 30A-70 |

| Compound | FD-MS |
|---|---|
| Sub 30A-1 | m/z = 525.25 (C₄₀H₃₁N = 525.7) |
| Sub 30A-2 | m/z = 485.21 (C [symbol] H₂₉N = 485.63) |
| Sub 30A-3 | m/z = 485.21 (C [symbol] H₂₇N = 485.63) |
| Sub 30A-4 | m/z = 459.2 (C₃₆H₂₅N = 459.59) |
| Sub 30A-5 | m/z = 409.18 (C₃₁H₂₉N = 409.53) |
| Sub 30A-6 | m/z = 409.18 (C [symbol] H [symbol] N = 409.53) |
| Sub 30A-7 | m/z = 499.19 (C₃₀H₂₂NO = 499.61) |
| Sub 30A-8 | m/z = 641.23 (C₄₇H₂₁NS = 641.83) |
| Sub 30A-9 | m/z = 624.26 (C₄₇H [symbol] N₂ = 624.79) |
| Sub 30A-10 | m/z = 485.21 (C [symbol] H₂₇N = 485.63) |
| Sub 30A-11 | m/z = 499.19 (C₃₇H₂₅NO = 499.61) |
| Sub 30A-12 | m/z = 681.21 (C₄₉H₃₁NOS = 681.85) |
| Sub 30A-13 | m/z = 674.27 (C₅₁H₃₄N₂ = 674.85) |
| Sub 30A-14 | m/z = 525.25 (C₄₀H₃₁N = 525.7) |
| Sub 30A-15 | m/z = 730.27 (C₅₂H₃₄N₄O = 730.87) |
| Sub 30A-16 | m/z = 515.17 (C₃₇H₂₅NS = 515.67) |
| Sub 30A-17 | m/z = 624.26 (C₄₇H₃₂N₂ = 624.79) |
| Sub 30A-18 | m/z = 525.25 (C₄₀H₃₁N = 525.7) |
| Sub 30A-19 | m/z = 499.19 (C₃₇H₂₅NO = 499.61) |
| Sub 30A-20 | m/z = 515.17 (C₃₇H₂₅NS = 515.67) |
| Sub 30A-21 | m/z = 490.25 (C₃₇H₂₂D₅N = 490.66) |
| Sub 30A-22 | m/z = 525.25 (C₄₀H₃₁N = 525.7) |
| Sub 30A-23 | m/z = 561.25 (C₄₃H₃₁N = 561.73) |
| Sub 30A-24 | m/z = 726.3 (C₅₅H₃₈N₂ = 726.92) |
| Sub 30A-25 | m/z = 409.18 (C₃₁H₂₃N = 409.53) |
| Sub 30A-26 | m/z = 561.25 (C₄₃H₃₁N = 561.73) |
| Sub 30A-27 | m/z = 601.28 (C₄₆H₃₅N = 601.79) |
| Sub 30A-28 | m/z = 525.25 (C₄₀H₃₁N = 525.7) |
| Sub 30A-29 | m/z = 525.25 (C₄₀H₃₁N = 525.7) |
| Sub 30A-30 | m/z = 525.25 (C₄₀H₃₁N = 525.7) |
| Sub 30A-31 | m/z = 601.28 (C₄₆H₃₅N = 601.79) |
| Sub 30A-32 | m/z = 651.29 (C₅₀H₃₇N = 651.85) |
| Sub 30A-33 | m/z = 575.26 (C₄₄H₃₃N = 575.76) |
| Sub 30A-34 | m/z = 535.23 (C₄₁H₂₉N = 535.69) |
| Sub 30A-35 | m/z = 525.25 (C₄₀H₃₁N = 525.7) |
| Sub 30A-36 | m/z = 561.25 (C₄₃H₃₁N = 561.73) |
| Sub 30A-37 | m/z = 485.21 (C₃₇H₂₇N = 485.63) |
| Sub 30A-38 | m/z = 566.28 (C₄₃H₂₆D₅N = 566.76) |
| Sub 30A-39 | m/z = 707.26 (C₅₂H₃₇NS = 707.94) |
| Sub 30A-40 | m/z = 459.2 (C₃₅H₂₅N = 459.59) |
| Sub 30A-41 | m/z = 409.18 (C₃₁H₂₃N = 409.53) |
| Sub 30A-42 | m/z = 485.21 (C₃₇H₂₇N = 485.63) |
| Sub 30A-43 | m/z = 485.21 (C₃₇H₂₇N = 485.63) |
| Sub 30A-44 | m/z = 661.28 (C₅₁H₃₅N = 661.85) |
| Sub 30A-45 | m/z = 624.26 (C₄₇H₃₂N₂ = 624.79) |
| Sub 30A-46 | m/z = 730.27 (C₅₂H₃₄N₄O = 730.87) |
| Sub 30A-47 | m/z = 515.17 (C₃₇H₂₅NS = 515.67) |
| Sub 30A-48 | m/z = 624.26 (C₄₇H₃₂N₂ = 624.79) |
| Sub 30A-49 | m/z = 525.25 (C₄₀H₃₁N = 525.7) |
| Sub 30A-50 | m/z = 499.19 (C₃₇H₂₅NO = 499.61) |
| Sub 30A-51 | m/z = 515.17 (C₃₇H₂₅NS = 515.67) |
| Sub 30A-52 | m/z = 490.25 (C₃₇H₂₂D₅N = 490.66) |
| Sub 30A-53 | m/z = 561.25 (C₄₃H₃₁N = 561.73) |
| Sub 30A-54 | m/z = 561.25 (C₄₃H₃₁N = 561.73) |
| Sub 30A-55 | m/z = 726.3 (C₅₅H₃₈N₂ = 726.92) |
| Sub 30A-56 | m/z = 561.25 (C₄₃H₃₁N = 561.73) |
| Sub 30A-57 | m/z = 561.25 (C₄₃H [symbol] N = 561.73) |
| Sub 30A-58 | m/z = 485.21 (C₃₇H₂₇N = 485.63) |
| Sub 30A-59 | m/z = 485.21 (C₃₇H₂₇N = 485.63) |
| Sub 30A-60 | m/z = 485.21 (C [symbol] H₂₇N = 485.63) |
| Sub 30A-61 | m/z = 525.25 (C₄₀H [symbol] N = 525.7) |
| Sub 30A-62 | m/z = 499.19 (C₃₇H₂₅NO = 499.61) |
| Sub 30A-63 | m/z = 499.19 (C₃₇H₂₇NO = 499.61) |
| Sub 30A-64 | m/z = 485.21 (C₃₇H₂₇N = 485.63) |
| Sub 30A-65 | m/z = 423.2 (C [symbol] H [symbol] N = 423.56) |
| Sub 30A-66 | m/z = 499.23 (C₃₈H₂₉N = 499.66) |
| Sub 30A-67 | m/z = 499.23 (C₄₀H₂₉N = 499.66) |
| Sub 30A-68 | m/z = 423.2 (C₃₉H₂₇N = 423.56) |
| Sub 30A-69 | m/z = 423.2 (C [symbol] H [symbol] N = 423.56) |
| Sub 30A-70 | m/z = 499.23 (C [symbol] H [symbol] N = 499.66) |

[symbol]

indicates data missing or illegible when filed

TABLE 10

| Compound | FD-MS | Compound | FD-MS |
|---|---|---|---|
| Sub 30B-1 | m/z = 245.97(C₁₂H7Br0 = 247.09) | Sub 30B-2 | m/z = 272.02(C₁₅H₁₂Br = 273.17) |
| Sub 30B-3 | m/z = 155.96(C [symbol] H₅Br = 157.01) | Sub 30B-4 | m/z = 261.95(C₁₂H [symbol] BrS = 263.15) |
| Sub 30B-5 | m/z = 272.02(C [symbol] H₁₃Br = 273.17) | Sub 30B-6 | m/z = 282(C₁₆H₁₁Br = 283.17) |
| Sub 30B-7 | m/z = 201.99(C₁₂H₉Br = 233.11) | Sub 30B-8 | m/z = 156.95(C [symbol] H [symbol] BrN = 158) |
| Sub 30B-9 | m/z = 311.01(C₁₅H [symbol] BrN₃ = 312.17) | Sub 30B-10 | m/z = 310.01(C [symbol] H₁₁BrN₃ = 311.18) |
| Sub 30B-11 | m/z = 283.99(C₁₄H₉BrN₂ = 285.14) | Sub 30B-12 | m/z = 255.99(C₁₄H₉Br = 257.13) |
| Sub 30B-13 | m/z = 360.03(C₂₀H₁₃BrN₂ = 361.24) | Sub 30B-14 | m/z = 334.01(C₁₈HₗₗBrN₂ = 335.2) |
| Sub 30B-15 | m/z = 339.97(C₁₆H₉BrN₂S = 341.23) | Sub 30B-16 | m/z = 322.04(C₁₉H₁₅Br = 323.23) |
| Sub 30B-17 | m/z = 394.04(C₂₅H₁₅Br = 395.3) | Sub 30B-18 | m/z = 396.05(C₂₅H₁₇Br = 397.32) |
| Sub 30B-19 | m/z = 249.98(C₁₂H₈BrF = 251.1) | Sub 30B-20 | m/z = 371.03(C₂₂H₁₄BrN = 372.27) |
| Sub 30B-21 | m/z = 398.07(C₂₅H₁₉Br = 399.33) | Sub 30B-22 | m/z = 348.05(C₂₁H₁₇Br = 349.27) |
| Sub 30B-23 | m/z = 255.99(C₁₄H₉Br = 257.13) | Sub 30B-24 | m/z = 237.02(C₁₂H₄D₅Br = 238.14) |
| Sub 30B-25 | m/z = 173.95(C₆H₄BrF = 175) | Sub 30B-26 | m/z = 308.02(C₁₈H₁₃Br = 309.21) |
| Sub 30B-27 | m/z = 272.02(C₁₅II₁₃Br = 273.17) | Sub 30B-28 | m/z = 313.05(C₁₈II₈D₅Br = 314.24) |
| Sub 30B-29 | m/z = 312.05(C₁₈H₁₇Br = 313.24) | Sub 30B-30 | m/z = 398.07(C₂₅H₁₉Br = 399.33) |
| Sub 30B-31 | m/z = 322(C₁₈H₁₁Br0 = 323.19) | Sub 30B-32 | m/z = 371.03(C₂₂H₁₄BrN = 372.27) |
| Sub 30B-33 | m/z = 245.97(Cₗ₂H₇Br0 = 247.09) | Sub 30B-34 | m/z = 261.95(C₁₂H₇BrS = 263.15) |
| Sub 30B-35 | m/z = 348.05(C₂₁II₁₇Br = 349.27) | Sub 30B-36 | m/z = 348.05(C₂₁II₁₇Br = 349.27) |
| Sub 30B-37 | m/z = 322(C₁₈H₁₁Br0 = 323.19) | Sub 30B-38 | m/z = 348.05(C₂₁H₁₇Br = 349.27) |
| Sub 30B-39 | m/z = 282(C₁₆H₁₁Br = 283.17) | Sub 30B-40 | m/z = 348.05(C₂₁Hₗ₇Br = 349.27) |
| Sub 30B-41 | m/z = 282(C₁₆H₁₁Br = 283.17) | Sub 30B-42 | m/z = 231.99(C₁₂H₉Br = 233.11) |
| Sub 30B-43 | m/z = 348.05(C₂₁H₁₇Br = 349.27) | Sub 30B-44 | m/z = 348.05(C₂₁H₁₇Br = 349.27) |
| Sub 30B-45 | m/z = 308.02(C₁₈H₁₈Br = 309.21) | | |

[symbol]

indicates data missing or illegible when filed

Illustration of Synthesis of Final Product 30

Synthesis of P-3

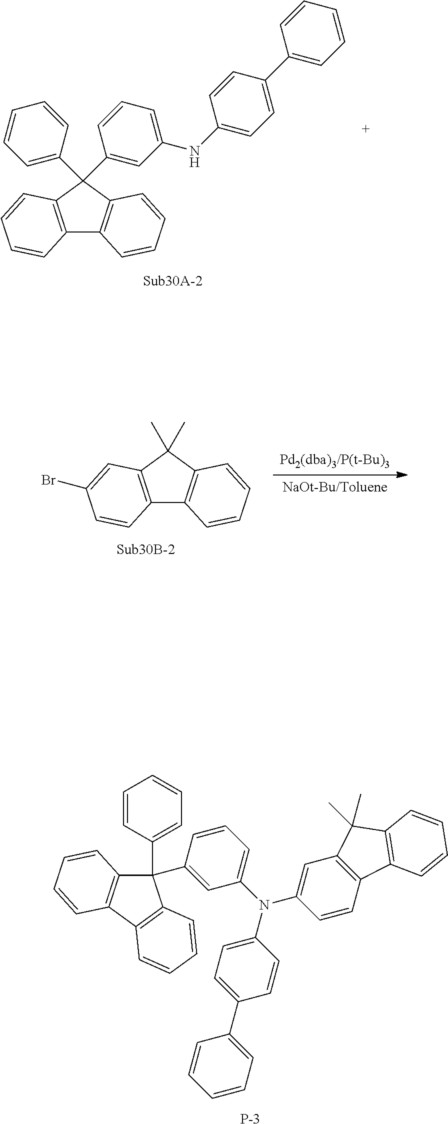

Sub 30A-2 (12 g, 24.7 mmol), Sub 30B-2 (7 g, 25.9 mmol), Pd₂(dba)₃ (0.68 g, 0.74 mmol), P(t-Bu)₃ (0.40 g, 1.98 mmol), NaOt-Bu (7.12 g, 74.13 mmol), and toluene (253 ml) were added to a round bottom flask, followed by stirring at 100° C. When the reaction was completed, extraction was performed with ether and water, and an organic layer was dried with MgSO₄ and concentrated. Afterwards, a resultant compound was subjected to silica gel column chromatography and recrystallization, thereby creating P-3 14.07 g (yield: 84%).

Synthesis of P-69

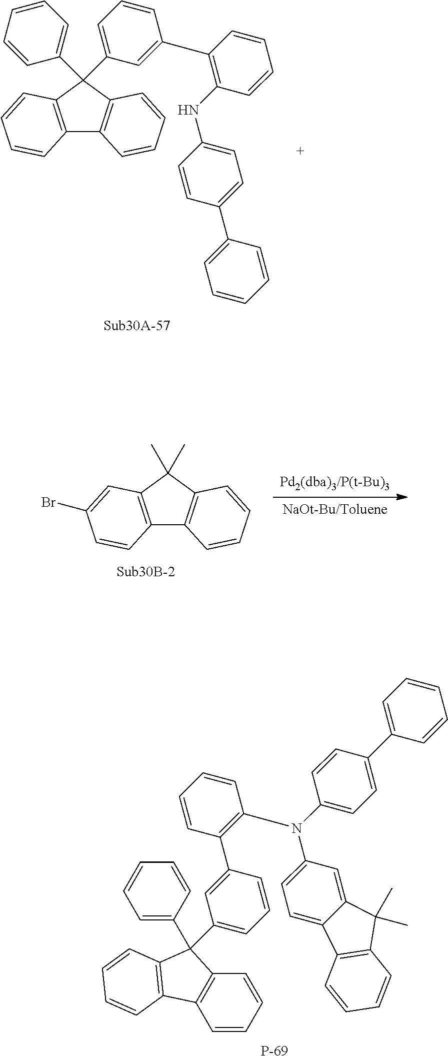

Sub 30A-57 (11 g, 19.58 mmol), Sub 30B-2 (5.62 g, 20.56 mmol), Pd₂(dba)₃ (0.54 g, 0.59 mmol), P(t-Bu)₃ (0.32 g, 1.57 mmol), NaOt-Bu (5.65 g, 58.75 mmol), and toluene (201 ml) were added to a round bottom flask, and then subjected to the same experimental method as in P-3, thereby creating P-69 12.11 g (yield: 82%).

Synthesis of P-71

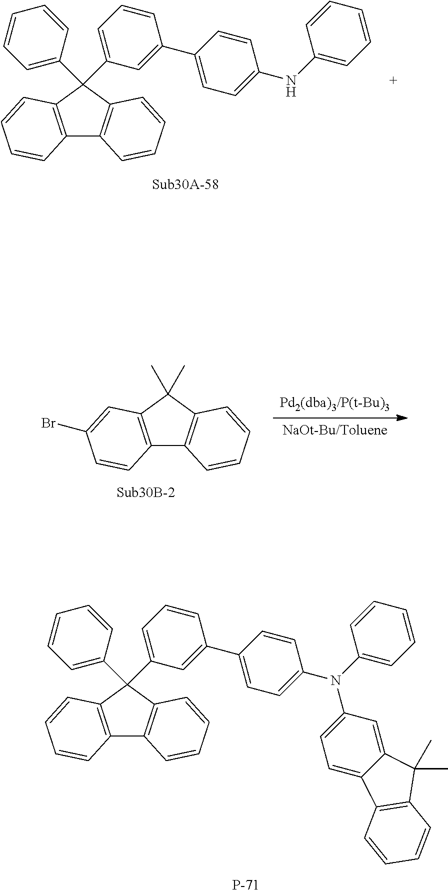

Sub 30A-58 (13 g, 26.77 mmol), Sub 30B-2 (7.68 g, 28.11 mmol), Pd₂(dba)₃ (0.74 g, 0.8 mmol), P(t-Bu)₃ (0.43 g, 2.14 mmol), NaOt-Bu (7.72 g, 80.31 mmol), and toluene (274 ml) were added to a round bottom flask, and then subjected to the same experimental method as in P-3, thereby creating P-71 14.34 g (yield: 79%).

Synthesis of P-80

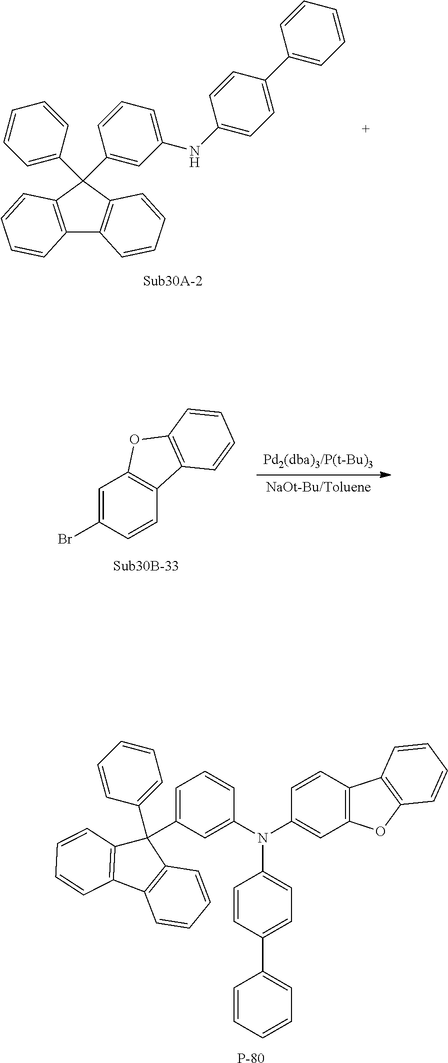

Sub 30A-2 (10 g, 20.59 mmol), Sub 30B-33 (5.34 g, 21.62 mmol), Pd₂(dba)₃ (0.57 g, 0.62 mmol), P(t-Bu)₃ (0.33 g, 1.65 mmol), NaOt-Bu (5.94 g, 61.78 mmol), and toluene (211 ml) were added to a round bottom flask, and then subjected to the same experimental method as in P-3, thereby creating P-80 10.87 g (yield: 81%).

Synthesis of P-88

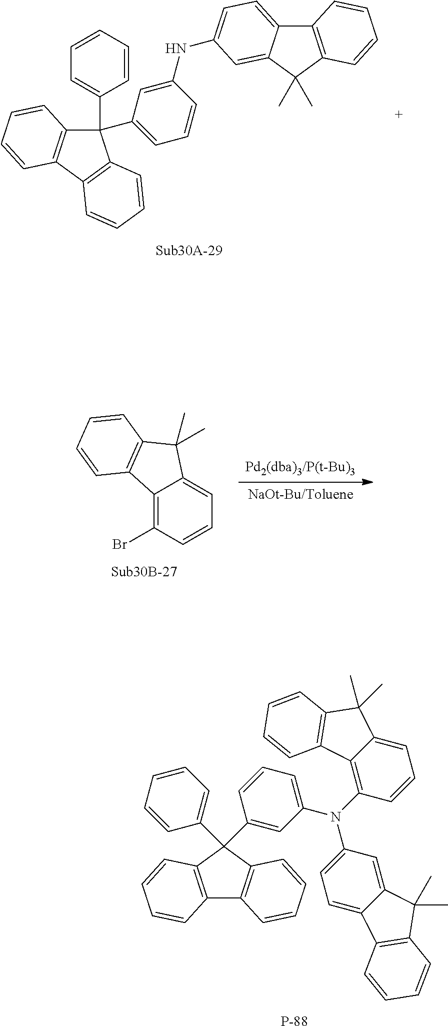

Sub 30A-29 (8.9 g, 16.93 mmol), Sub 30B-27 (4.86 g, 17.78 mmol), Pd₂(dba)₃ (0.47 g, 0.51 mmol), P(t-Bu)₃ (0.27 g, 1.35 mmol), NaOt-Bu (4.88 g, 50.79 mmol), and toluene (174 ml) were added to a round bottom flask, and then subjected to the same experimental method as in P-3, thereby creating P-88 9.72 g (yield: 80%)

Synthesis of P-95

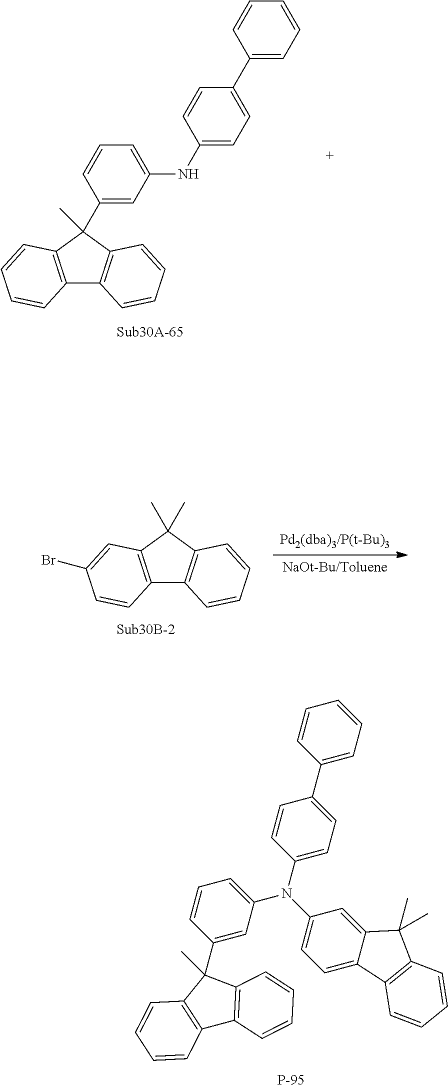

Sub 30A-65 (7.8 g, 18.42 mmol), Sub 30B-2 (5.28 g, 19.34 mmol), Pd₂(dba)₃ (0.51 g, 0.55 mmol), P(t-Bu)₃ (0.30 g, 1.47 mmol), NaOt-Bu (5.31 g, 55.25 mmol), and toluene (189 ml) were added to a round bottom flask, and then subjected to the same experimental method as in P-3, thereby creating P-95 8.39 g (yield: 74%).

Synthesis of P-98

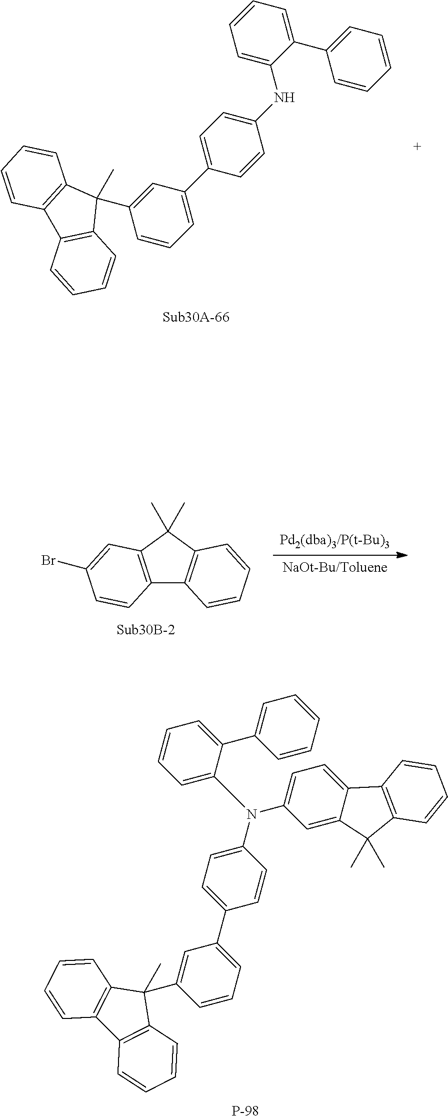

Sub 30A-66 (15 g, 30.02 mmol), Sub 30B-2 (8.61 g, 31.52 mmol), Pd₂(dba)₃ (0.82 g, 0.9 mmol), P(t-Bu)₃ (0.49 g, 2.40 mmol), NaOt-Bu (8.66 g, 90.06 mmol), and toluene (308 ml) were added to a round bottom flask, and then subjected to the same experimental method as in P-3, thereby creating P-98 14.75 g (yield: 71%).

TABLE 11

| Compound | FD-MS | Compound | FD-MS |
|---|---|---|---|
| P-1 | m/z = 691.29(C [symbol] H₃₇N0 = 691.37) | P-2 | m/z = 651.26(C [symbol] H [symbol] N0 = 651.61) |
| P-3 | m/z = 677.31(C [symbol] H [symbol] N = 677.80) | P-4 | m/z = 651.26(C [symbol] H [symbol] N0 = 65l.81) |
| P-5 | m/z = 641.22(C [symbol] H [symbol] NS = 641.33) | P-6 | m/z = 700.29(C [symbol] H [symbol] N₂ = 700.89) |
| P-7 | m/z = 601.28(C [symbol] H N = 601.79) | P-8 | m/z = 651.26(C [symbol] H [symbol] N0 = 651.81) |
| P-9 | m/z = 843.3(C [symbol] H [symbol] NS = 844.09) | P-10 | m/z = 701.28(C [symbol] H [symbol] N₃ = 701.87) |
| P-11 | m/z = 677.31(C₅₂H₃₉N = 677.89) | P-12 | m/z = 729.28(C₅₃H₃₅N₃) = 729.88) |
| P-13 | m/z = 912.29(C₆₄H₄₀N₃ = 913.11) | P-14 | m/z = 878.34(C₆₅H₄₂N₄ = 879.08) |
| P-15 | m/z = 805.35(C₆₀H₄₃N₃ = 806.03) | P-16 | m/z = 906.34(C₆₆H₄₂N₄0 = 907.09) |
| P-17 | m/z = 769.26(C₅₅H₃₅N₃S = 769.97) | P-18 | m/z = 884.3(C₆₃H₁₀N₄S = 885.1) |
| P-19 | m/z = 767.36(C₄₈H₄₅N = 768.02) | P-20 | m/z = 815.32(C₆₂H₄₁N0 = 816.02) |
| P-21 | m/z = 829.28(C₆₂H₃₉NS = 830.06) | P-22 | m/z = 781.35(C₅₉H₃₅D₅N₂ = 782.01) |
| P-23 | m/z = 695.3(C₅₂H₃₈FN = 695.88) | P-24 | m/z = 753.34(C₅₈H₃₄N = 753.99) |
| P-25 | m/z = 803.36(C₆₂H₄₅N = 804.05) | P-26 | m/z = 829.37(C₆₄H₄₇N = 830.09) |
| P-27 | m/z = 918.4(C₇₀II₅₀N₂ = 919.18) | P-28 | m/z = 601.28(C₄₆II₃₅N = 601.79) |
| P-29 | m/z = 677.31(C₅₂H₃₉N = 677.89) | P-30 | m/z = 753.34(C₅₈H₄₃N = 753.99) |
| P-31 | m/z = 701.31(C₅₄H₃₉N = 701.91) | P-32 | m/z = 677.31(C₅₂H₃₉N = 677.89) |
| P-33 | m/z = 677.31(C₅₂H₃₉N = 677.89) | P-34 | m/z = 682.34(C₅₂H₃₄D₅N = 682.92) |
| P-35 | m/z = 701.31(C₅₄H₃₉N = 701.91) | P-36 | m/z = 619.27(C₄₆H₃₄FN = 619.78) |
| P-37 | m/z = 753.34(C₅₈H₄₃N = 753.99) | P-38 | m/z = 879.39(C₆₈H₄₉N = 880.15) |
| P-39 | m/z = 701.31(C₅₄H₃₉N = 701.91) | P-40 | m/z = 651.29(C₅₀II₃₇N = 651.85) |
| P-41 | m/z = 727.32(C₅₆H₄₁N = 727.95) | P-42 | m/z = 758.37(C₅₈H₃₈D₅N = 759.02) |
| P-43 | m/z = 757.37(C₅₈H₄₇N = 758.02) | P-44 | m/z = 753.34(C₅₈H₄₃N = 753.99) |
| P-45 | m/z = 803.36(C₆₂H₄₅N = 804.05) | P-46 | m/z = 834.4(C₆₄H₄₂D₅N = 835.12) |
| P-47 | m/z = 883.33(C₆₆H₄₅NS = 884.15) | P-48 | m/z = 651.26(C₄₉H_{3S}N0 = 651.81) |
| P-49 | m/z = 641.22(C₄₇H₃₁NS = 641.83) | P-50 | m/z = 700.29(C₅₃H₃₆N₂ = 700.89) |
| P-51 | m/z = 677.31(C₅₂H₃₉N = 677.89) | P-52 | m/z = 651.26(C₄₉H₃₃N0 = 651.81) |
| P-53 | m/z = 843.3(C₆₃H₄₁NS = 844.09) | P-54 | m/z = 701.28(C₅₂H₃₅N₃ = 701.87) |
| P-55 | m/z = 677.31(C₅₂H₃₉N = 677.89) | P-56 | m/z = 906.34(C₆₆H₄₂N₄0 = 907.09) |
| P-57 | m/z = 769.26(C₅₅H₃₅N₃S = 769.97) | P-58 | m/z = 884.3(C₆₃H₄₀N₄S = 885.1) |
| P-59 | m/z = 767.36(C₅₉H₄₅N = 768.02) | P-60 | m/z = 815.32(C₂₂H₄₁N0 = 816.02) |
| P-61 | m/z = 829.28(C₆₂H₃₉NS = 830.06) | P-62 | m/z = 781.35(C₅₉H₃₅D₅N₂ = 782.01) |
| P-63 | m/z = 695.3(C₅₂H₃₈FN = 695.88) | P-64 | m/z = 753.34(C [symbol] H [symbol] N = 753.99) |
| P-65 | m/z = 803.36(C₅₂H₄₃N = 804.05) | P-66 | m/z = 829.37(C [symbol] H₄₇N = 830.09) |
| P-67 | m/z = 918.4(C [symbol] H [symbol] N = 919.18) | P-68 | m/z = 753.34(C [symbol] H [symbol] N = 753.99) |
| P-69 | m/z = 753.34(C₅₈H₄₃N = 753.99) | P-70 | m/z = 601.28(C₄₆H₃₅N = 601.79) |
| P-71 | m/z = 677.31(C [symbol] H [symbol] N = 677.89) | P-72 | m/z = 677.31(C [symbol] H [symbol] N = 677.89) |
| P-73 | m/z = 677.31(C [symbol] H [symbol] N = 677.89) | P-74 | m/z = 677.31(C [symbol] H [symbol] N = 677.89) |
| P-75 | m/z = 677.31(C [symbol] H [symbol] N = 677.89) | P-76 | m/z = 717.34(C₅₅H₄₃N = 717.96) |
| P-77 | m/z = 753.34(C [symbol] H [symbol] N = 753.99) | P-78 | m/z = 691.29(C₅₂H₃₇N0 = 691.87) |
| P-79 | m/z = 767.32(C [symbol] H [symbol] N0 = 767.97) | P-80 | m/z = 651.26(C₄₉H₃₃N0 = 651.81) |
| P-81 | m/z = 665.24(C [symbol] H [symbol] N0₂ = 665.79) | P-82 | m/z = 691.29(C₅₂H₃₇N0 = 691.87) |
| P-83 | m/z = 717.34(C [symbol] H [symbol] N = 717.96) | P-84 | m/z = 677.31(C [symbol] H [symbol] N = 677.89) |
| P-85 | m/z = 691.29(C [symbol] H [symbol] N = 691.87) | P-86 | m/z = 753.34(C [symbol] H [symbol] N = 753.99) |
| P-87 | m/z = 727.32(C [symbol] H [symbol] N = 727.95) | P-88 | m/z = 717.34(C [symbol] H [symbol] N = 717.96) |
| P-89 | m/z = 677.31(C [symbol] H [symbol] N = 677.89) | P-90 | m/z = 753.34(C [symbol] H [symbol] N = 753.99) |
| P-91 | m/z = 727.32(C [symbol] H [symbol] N = 727.98) | P-92 | m/z = 793.37(C [symbol] H [symbol] N = 794.05) |
| P-93 | m/z = 753.34(C [symbol] H [symbol] N = 753.99) | P-94 | m/z = 753.34(C [symbol] H [symbol] N = 753.99) |
| P-95 | m/z = 615.29(C₄₇H₃₇N = 815.82) | P-96 | m/z = 615.29(C₄₇H₃₇N = 615.82) |
| P-97 | m/z = 691.32(C [symbol] H [symbol] N = 691.92) | P-98 | m/z = 691.32(C [symbol] H [symbol] N = 691.92) |
| P-99 | m/z = 691.32(C [symbol] H [symbol] N = 691.92) | P-100 | m/z = 691.32(C [symbol] H [symbol] N = 691.92) |
| P-101 | m/z = 691.32(C [symbol] H [symbol] N = 691.92) | P-102 | m/z = 691.32(C [symbol] H [symbol] N = 691.92) |

[symbol]

indicates data missing or illegible when filed

Evaluation of Fabrication of Organic Electronic Device

When the organic electronic device according to the present disclosure is a top-emission device and the anode is formed on the substrate before the organic material layer and the cathode are formed, the anode material may be implemented as not only a transparent material but also an opaque material having superior light reflectivity.

When the organic electronic device according to the present disclosure is a bottom-emission device and the anode is formed on the substrate before the organic material layer and the cathode are formed, the anode material should be implemented as a transparent material or, when formed of an opaque material, provided as a thin film as thin as possible so as to be transparent.

Hereinafter, the following Examples will be proposed by fabricating a top-emission tandem organic electronic device, but embodiments of the present disclosure are not limited thereto. The tandem organic electronic device according to an embodiment of the present disclosure is fabricated such that a plurality of stacks are connected through one or more charge generation layers. Although the same compound has been used for the hole transport layers of each of the three stacks in the tandem organic electronic device according to an embodiment of the present disclosure, the present disclosure is not limited thereto.

### [Example 136] Tandem Organic Electronic Device with Three Stacks Connected

A tandem organic electronic device including three stacks connected were fabricated with a structure of first electrode (anode)/first hole transport region/first emission layer/first electron transport region/charge generation layer/second hole transport region/second emission layer/second electron transport region/charge generation layer/third hole transport region/third emission layer/third electron transport region/electron injection layer/second electrode (cathode).

Specifically, a hole injection layer was formed by vacuum-depositing 4,4′,4″-Tris[2-naphthyl(phenyl)amino]triphenylamine (hereinafter, abbreviated as TNATA) at a thickness of 60 nm an anode formed on a glass substrate, a first hole transport layer of a first stack was formed at a thickness of 11 nm (35% of a total thickness 30 nm) by doping compound P-3 represented by Formula 1 of the present disclosure (hereinafter, referred to as first HTM) with HATCN serving as a doping material, and then P-3 represented by Formula 1 of the present disclosure was formed at a thickness of 19 nm on the first hole transport layer. Subsequently, a first emission layer having a thickness of 20 nm was deposited on the first hole transport layer using DPVBi as a host and 5% by weight of BCzVBi as a dopant. An electron transport layer was formed at a thickness of 30 nm using Alq3. Afterwards, a charge generation layer was formed by doping Bphen with 2% of Li for connection to a second stack. In addition, a second hole transport layer of the second stack was formed at a thickness of 14 nm (25% of a total thickness of 55 nm) by doping compound P-3 represented by Formula 1 of the present disclosure (hereinafter, referred to as second HTM) with 10% of HATCN serving as a doping material, and then P-3 represented by Formula 1 of the present disclosure was formed at a thickness of 41 nm on the second hole transport layer. Afterwards, as described above, a second emission layer, a second electron transport region, and a charge generation layer were formed sequentially. Finally, a third hole transport layer of a third stack was formed at a thickness of 10 nm (20% of a total thickness of 50 nm) by doping compound P-3 represented by Formula 1 of the present disclosure (hereinafter, referred to as third HTM) with 10% of HATCN serving as a doping material, and then P-3 represented by Formula 1 of the present disclosure was formed at a thickness of 40 nm on the third hole transport layer. After a third emission layer and a third electron transport region are sequentially deposited as described above, an electron injection layer of Liq was formed at a thickness of 1.5 nm, and then a cathode was formed by depositing Ag:Mg at a thickness of 150 nm. In this manner, the tandem organic electronic device was fabricated.

Electroluminescence (EL) properties of organic electronic emission devices according to Examples and Comparative Examples fabricated in this manner were measured using PR-650 available from Photo Research, Inc. by applying a forward bias DC voltage to the devices, and as a result of the measurement, T95 lifespans of the devices were tested using lifetime test equipment available from Mcscience Inc. The following table illustrates the results of the fabrication and test of the devices.

### Examples 137 to 160

Organic electronic emission devices were fabricated in the same method as Example 136, except that compounds illustrated in the following Table 12 were used as hole transport materials of the first to third stacks.

### Comparative Examples 10 and 11

Organic electronic emission devices were fabricated in the same method as Example, except that only the single stack was formed and the following ref 1 and ref 2 were used as hole transport materials.

[Ref 1 and Ref 2]

TABLE 12

| | | | | | | | | | | ref 1 |
|---|---|---|---|---|---|---|---|---|---|---|
| 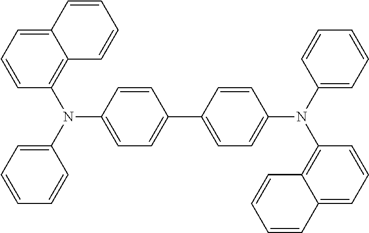 [SMILES: c1ccc(N(c2ccc(-c3ccc(N(c4ccccc4)c4cccc5ccccc45)cc3)cc2)c2cccc3ccccc23)cc1] | | | | | | | | | | |
| | | | | | | | | | | ref 2 |
| 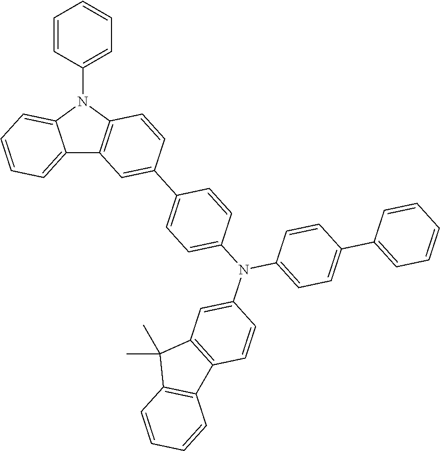 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] | | | | | | | | | | |
| | 1^{st} | 2^{nd} | 3^{rd} | Driving | Current | Luminance | Efficiency | | CIE | CIE |
| | HTM | HTM | HTM | voltage (V) | (mA/cm²) | (csd/m²) | (cd/A) | T (95) | (x) | (y) |
| Comp. | ref 1 | — | — | 15.4 | 18.8 | 1500 | 8 | 90.1 | 0.137 | 0.100 |
| Ex. 10 | | | | | | | | | | |
| Comp. | ref 2 | — | — | 13 | 15 | 1500 | 10 | 95.4 | 0.135 | 0.100 |
| Ex. 11 | | | | | | | | | | |

| | | | | | | | | |
|---|---|---|---|---|---|---|---|---|
| Ex. 136 | P-3 | 10.1 | 6.2 | 1500 | 24.2 | 162.1 | 0.127 | 0.100 |
| Ex. 137 | P-16 | 12 | 9.4 | 1500 | 16 | 130.4 | 0.125 | 0.100 |
| Ex. 138 | P-18 | 12 | 9.1 | 1500 | 16.5 | 131.7 | 0.126 | 0.100 |
| Ex. 139 | P-24 | 10.4 | 6.8 | 1500 | 22 | 158.3 | 0.126 | 0.100 |
| Ex. 140 | P-32 | 10.9 | 7.3 | 1500 | 20.5 | 146.1 | 0.125 | 0.100 |
| Ex. 141 | P-34 | 10.1 | 6.4 | 1500 | 23.3 | 161.3 | 0.125 | 0.100 |
| Ex. 142 | P-59 | 11.9 | 8.9 | 1500 | 16.8 | 132.4 | 0.127 | 0.100 |
| Ex. 143 | P-68 | 10.3 | 6.9 | 1500 | 21.6 | 160.7 | 0.127 | 0.100 |
| Ex. 144 | P-69 | 10.3 | 6.9 | 1500 | 21.8 | 160.1 | 0.126 | 0.100 |
| Ex. 145 | P-70 | 11.7 | 7.9 | 1500 | 19.1 | 139.8 | 0.127 | 0.100 |
| Ex. 146 | P-71 | 10 | 6.1 | 1500 | 24.4 | 162.5 | 0.127 | 0.100 |
| Ex. 147 | P-77 | 10.9 | 8.2 | 1500 | 18.2 | 155.7 | 0.127 | 0.100 |
| Ex. 148 | P-80 | 11.2 | 8.1 | 1500 | 18.5 | 157 | 0.126 | 0.100 |
| Ex. 149 | P-81 | 11.8 | 8.7 | 1500 | 17.3 | 135.6 | 0.126 | 0.100 |
| Ex. 150 | P-82 | 11.3 | 7.7 | 1500 | 19.4 | 144 | 0.126 | 0.100 |
| Ex. 151 | P-84 | 10.1 | 6.4 | 1500 | 23.6 | 161.8 | 0.126 | 0.100 |
| Ex. 152 | P-85 | 10.8 | 7.6 | 1500 | 19.7 | 153.2 | 0.126 | 0.100 |
| Ex. 153 | P-86 | 11.5 | 7.1 | 1500 | 21 | 138.7 | 0.125 | 0.100 |
| Ex. 154 | P-88 | 11.7 | 8.5 | 1500 | 7.7 | 141.1 | 0.126 | 0.100 |
| Ex. 155 | P-90 | 10.6 | 7.9 | 1500 | 19.1 | 152.5 | 0.126 | 0.100 |
| Ex. 156 | P-91 | 11.3 | 7.1 | 1500 | 21.1 | 148.6 | 0.125 | 0.100 |
| Ex. 157 | P-95 | 9.8 | 6.6 | 1500 | 22.9 | 165.2 | 0.125 | 0.100 |
| Ex. 158 | P-96 | 9.9 | 6.7 | 1500 | 22.5 | 164 | 0.126 | 0.100 |
| Ex. 159 | P-98 | 9.5 | 6 | 1500 | 25 | 169.6 | 0.126 | 0.100 |
| Ex. 160 | P-100 | 9.6 | 6.1 | 1500 | 24.7 | 167.4 | 0.126 | 0.100 |

As seen from the result of Table 12, it can be appreciated that, when the tandem organic light-emitting devices each including three stacks were fabricated using the compound represented by Formula 1 of the present disclosure as the hole transport material (Examples 136 to 160), the electrical characteristics of the devices were improved than when organic light-emitting devices each including a single stack using the ref 1 compound and the ref 2 compound as the hole transport materials (Comparative Examples 10 and 11). Describing in detail, in Examples 136 to 160 and Comparative Examples 10 and 11, the materials of the hole transport layers were doped with the doping materials at the same in the thickness, whereas different numbers of stacks were connected. As in Examples 136 to 160, it can be appreciated that the efficiency and lifespan among the device characteristics were significantly improved with increases in the number of the stacks connected. It is considered that the efficiency and lifespan were improved proportionally to increases in the number of the stacks, due to the multiphoton emission structure in which excitons are generated to emit light energy in each of the stacks.

It can also be seen that the values of the color coordinates (CIE x) gradually decrease, due to the device structure including three stacks as in Examples of the present disclosure. It is considered that the color purity was improved as the full width at half maximum (FWHM) of an emission wavelength was reduced with increases in the number of the stacks

In the meantime, it can be seen that, when the compound represented by Formula 1 of the present disclosure was used as the first hole transport layer material, the device characteristics were more improved than when the ref 1 material or the ref 2 material including N were used as first hole transport layer. When the compound of the present disclosure was used as the hole transport layer material, an appropriate number of holes can be efficiently moved in the emission layer to balance holes and electrons in the emission layer and prevent degradations in the interface of the emission layer, thereby increasing the efficiency and lifespan.

### [Examples 161 and 164] Tandem Organic Electronic Device with Three Stacks Connected

Tandem organic light-emitting devices were fabricated in the same method as Example 136, except that the compounds P-3 and P-71 were used as the hole transport materials of the first to third stacks as illustrated in the following Table 13 and the portions corresponding to 15% of the thickness of the hole transport layers were doped with HATCN, each of the hole transport layers being 50 nm thick.

### Examples 162 and 165

Tandem organic light-emitting devices were fabricated in the same method as Example 136, except that the compounds P-3 and P-71 were used as the hole transport materials of the first to third stacks as illustrated in the following Table 13 and the portions corresponding to 20% of the thickness of the hole transport layers were doped with HATCN, each of the hole transport layers being 50 nm thick.

### Examples 163 and 166

Tandem organic light-emitting devices were fabricated in the same method as Example 136, except that the compounds P-3 and P-71 were used as the hole transport materials of the first to third stacks as illustrated in the following Table 13 and the portions corresponding to 25% of the thickness of the hole transport layers were doped with HATCN, each of the hole transport layers being 50 nm thick.

### Comparative Examples 12 and 14

Tandem organic light-emitting devices were fabricated in the same method as Example 136, except that the compounds P-3 and P-71 were used as the hole transport materials of the first to third stacks as illustrated in the following Table 13 and the portions corresponding to 10% of the thickness of the hole transport layers were doped with HATCN, each of the hole transport layers being 50 nm thick.

### Comparative Examples 13 and 15

Tandem organic light-emitting devices were fabricated in the same method as Example 136, except that the compounds P-3 and P-71 were used as the hole transport materials of the first to third stacks as illustrated in the following Table 13 and the portions corresponding to 55% of the thickness of the hole transport layers were doped with HATCN, each of the hole transport layers being 50 nm thick.

TABLE 13

| | 1^{st} to 3^{rd} | Thickness | Driving | Current | Luminance | Efficiency | Lifespan |
|---|---|---|---|---|---|---|---|
| | HTM | ratio¹⁾ | voltage (V) | (mA/cm²) | (csd/m²) | (cd/A) | T(95) |
| Comp. Ex. 12 | P-3 | 10% | 12.5 | 9 | 1500 | 16.7 | 103.7 |
| Comp. Ex. 13 | P-3 | 55% | 13 | 9.5 | 1500 | 15.8 | 104.8 |
| Comp. Ex. 14 | P-71 | 10% | 12.1 | 8.8 | 1500 | 17 | 104.2 |
| Comp. Ex. 15 | P-71 | 55% | 12.7 | 9.3 | 1500 | 16.2 | 105.4 |
| Ex. 161 | P-3 | 15% | 10.5 | 6.2 | 1500 | 24.3 | 162.4 |
| Ex. 162 | P-3 | 20% | 10.3 | 6.1 | 1500 | 24.7 | 162.9 |
| Ex. 163 | P-3 | 25% | 10.2 | 6 | 1500 | 25.1 | 163.2 |
| Ex. 164 | P-71 | 15% | 10.3 | 6.1 | 1500 | 24.6 | 162.7 |
| Ex. 165 | P-71 | 20% | 10.2 | 6 | 1500 | 24.9 | 163.3 |
| Ex. 166 | P-71 | 25% | 9.8 | 5.9 | 1500 | 25.3 | 163.9 |

Notes)
Thickness ratio¹⁾: Thickness ratio (% of thickness of hole transport layer)

As illustrated in Table 13, the tandem devices were fabricated and measured by varying the ratio of the portion of the hole transport layer doped with the doping material with respect to the thickness of the hole transport layer in each of the first to third stacks according to the present disclosure. In the illustrative description of the compounds, P-3 and P-71 were taken as examples. As seen from the results of Table 13, it can be appreciated that, when the hole transport layer is doped with the doping material so that the ratio of the thickness of the hole transport layer doped with the doping material is less than 15% of the total thickness of the hole transport layer or is greater than 50% of the total thickness of the hole transport layer, the results regarding the driving voltage, efficiency, and lifespan of the devices gradually decline compared to the results regarding Examples 161 to 166, in which the hole transport layer was doped with the doping material at 15%, 20%, and 25% ratios. The results depend on the thickness of the portion of the hole transport layer doped with the doping material, i.e., are proportional to the weight ratio of the doping material added to the hole transport layer. When the portion of the hole transport layer doped with the doping material is too thin, the generation of holes and electric charges is insignificant, and holes are not properly injected into the emission layer. Consequently, the device characteristics may be degraded, which is problematic. In contrast, when the thickness of the portion of the hole transport layer doped with the doping material is too thick, there may be problems in terms of the occurrence of short-circuit or an increase in the total cost consumed for the fabrication of the devices.

The above description is only intended to illustrate the present disclosure, and those having ordinary knowledge in the art to which the present disclosure pertains could make various modifications without departing from the essential features of the present disclosure. The foregoing embodiments disclosed herein shall be interpreted as being illustrative, while not being limitative, of the principle and scope of the present disclosure. It should be understood that the scope of the present disclosure shall be defined by the appended Claims and all of their equivalents fall within the scope of the present disclosure.

### DESCRIPTION OF REFERENCE NUMERALS OF DRAWINGS

110: first electrode
120: second electrode
130: organic material layer
141: first stack
142: second stack
143: third stack
144: fourth stack
150: charge generation layer
160: capping layer

### CROSS-REFERENCE TO RELATED APPLICATION

This application claims under 35 U.S.C. § 119(a) the benefit of priority to Korean Patent Application No. 10-2019-0094551, filed on Aug. 2, 2019, Korean Patent Application No. 10-2020-0047662, filed on Apr. 20, 2020, and Korean Patent Application No. 10-2020-0082255, and filed on Jul. 3, 2020, respectively, which are hereby incorporated by reference for all purposes as if fully set forth herein. In addition, when this application claims priority in countries other than the U.S.A. on the same basis, the entire contents of which are hereby incorporated by reference.

1. An organic electronic device comprising:
a first electrode;
a second electrode; and
an organic material layer positioned between the first electrode and the second electrode and comprising a first stack, a second stack, and a third stack,
wherein the first stack comprises a first hole transport region, a first emission layer, and a first electron transport region,
the first hole transport region comprises a first hole transport layer and a first auxiliary emission layer,
the first hole transport layer or the first auxiliary emission layer comprises a first compound represented by the following Formula 1,
the thickness of the first hole transport layer ranges from 250 Å to 700 Å, and
10% to 50% of the thickness of the first hole transport layer is doped with a first doping material, 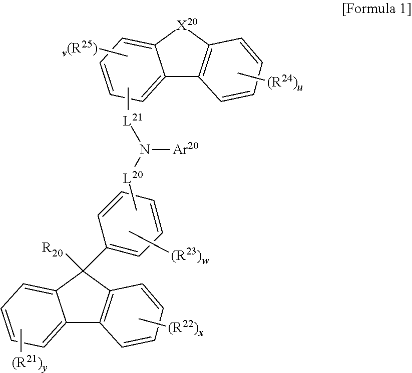
wherein, in Formula 1,
each of R²⁰ to R²⁵ is independently selected from the group consisting of i) deuterium; a halogen; a C₆-C₃₀ aryl group; a fluorenyl group; a C₂-C₃₀ heterocyclic group including at least one heteroatom of O, N, S, Si, or P; a fused ring group of a C₃-C₃₀ aliphatic ring and a C₆-C₃₀ aromatic ring; a C₁-C₃₀ alkyl group; a C₂-C₂₀ alkenyl group; a C₂-C₂₀ alkynyl group; a C₁-C₃₀ alkoxyl group; or a C₆-C₃₀ aryloxy group, or ii) a plurality of R²¹s, a plurality of R²²s, a plurality of R²³s, a plurality of R²⁴s, and a plurality of R²⁵s are bonded to each other to form rings, respectively,
v is an integer of one of 0 to 3,
each of u, w, x, and y is independently an integer of one of 0 to 4,
each of L²⁰ and L²¹ is independently selected from the group consisting of a single bond; a fluorenylene group; a C₆-C₃₀ arylene group; or a C₃-C₃₀ heterocyclic group including at least one hetero atom of O, N, S, Si, or P,
Ar²⁰ is a C₆-C₃₀ aryl group or a C₃-C₃₀ heterocyclic group including at least one hetero atom of O, N, S, Si, or P,
X²⁰ is O, S, NR′, or CR′ R″,
R′ and R″ are respectively and independently selected from the group consisting of a C₁-C₃₀ alkyl group; a C₅-C₃₀ aryl group; or a C₃-C₃₀ heterocyclic group including at least one hetero atom of O, N, S, Si, or P, or the R′ and R″ are bonded to each other to form spiro compounds,
in Formula 1, each of the aryl group, the fluorenyl group, the heterocyclic group, the fused ring group, the alkyl group, the alkenyl group, the alkynyl group, the alkoxyl group, the aryloxy group, the arylene group, and the fluorenylene group is further substituted with one or more substituents selected from the group consisting of deuterium; a halogen group; a C₁-C₂₀ alkoxyl group; a C₁-C₂₀ alkyl group; a C₂-C₂₀ alkenyl group; a C₂-C₂₀ alkynyl group; a C₆-C₂₅ aryl group; a C₅-C₂₅ aryl group substituted with deuterium; a fluorenyl group; a C₂-C₂₀ heterocyclic group; or a C₃-C₂₀ cycloalkyl group, and
each of the further substituted substituents is capable of being further substituted with one or more substituents selected from the group consisting of deuterium; a halogen group; a C₁-C₂₀ alkoxyl group; a C₁-C₂₀ alkyl group; a C₂-C₂₀ alkenyl group; a C₂-C₂₀ alkynyl group; a C₆-C₂₅ aryl group; a C₅-C₂₅ aryl group substituted with deuterium; a fluorenyl group; a C₂-C₂₀ heterocyclic group; or a C₃-C₂₀ cycloalkyl group, and the substituents are bonded to each other to form a ring.

2. The device of claim 1, wherein each of the first hole transport layer and the first auxiliary emission layer of the first stack comprises the first compound represented by Formula 1.

3. The device of claim 1, wherein the first compound is represented by the following Formula 2: 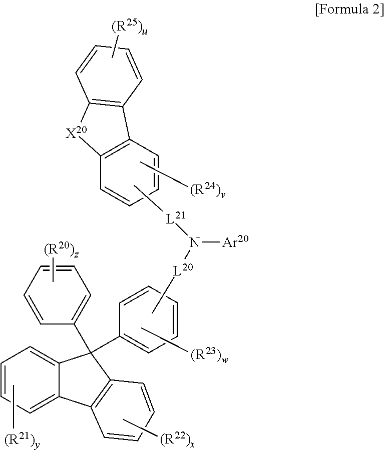
wherein, in Formula 2,
z is an integer from 0 to 5, and
u, v, w, x, y, R²⁰ to R²⁵, L²⁰, L²¹, Ar²⁰, and X²⁰ are the same as those defined in claim 1.

4. The device of claim 1, wherein the first compound is represented by one of the following Formulas 3 to 5:
[Formulas 3, 4, and 5] 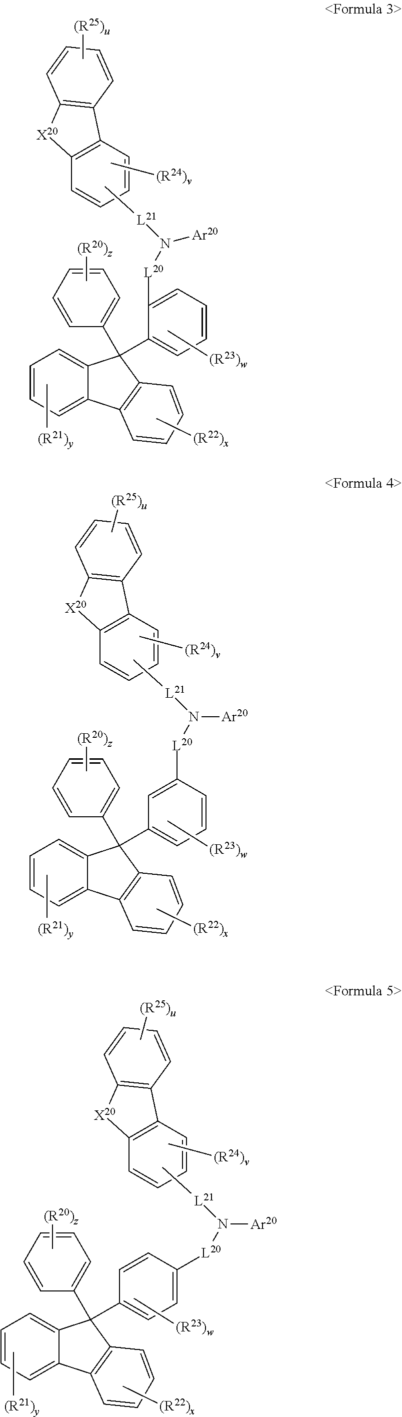
wherein, in Formulas 3 to 5,
u, v, w, x, y, R²⁰ to R²⁵, L²⁰, L²¹, Ar²⁰, and X²⁰ are the same as those defined in claim 1.

5. The device of claim 1, wherein the first compound is represented by one of the following Formulas 6 to 9:
[Formulas 6, 7, 8, and 9] 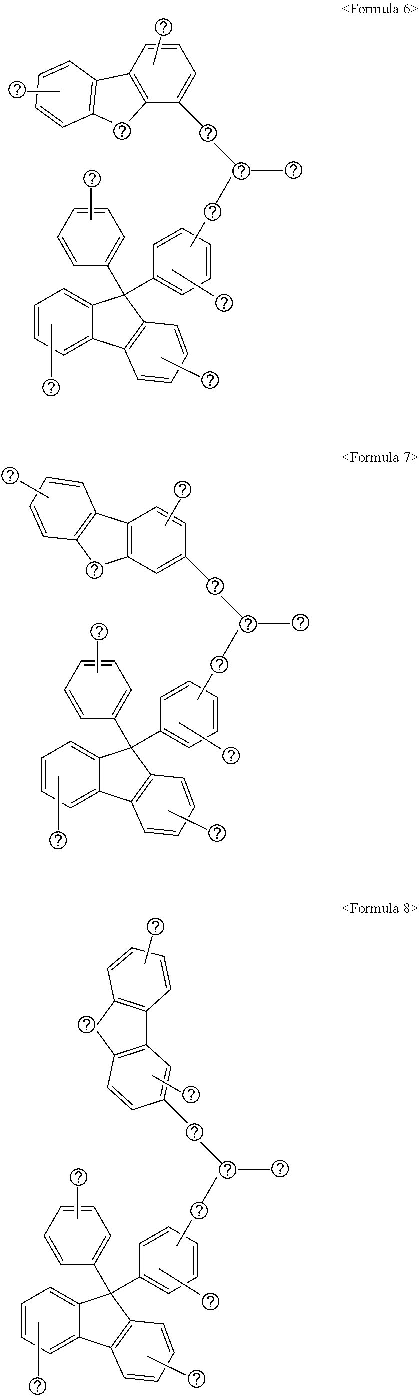 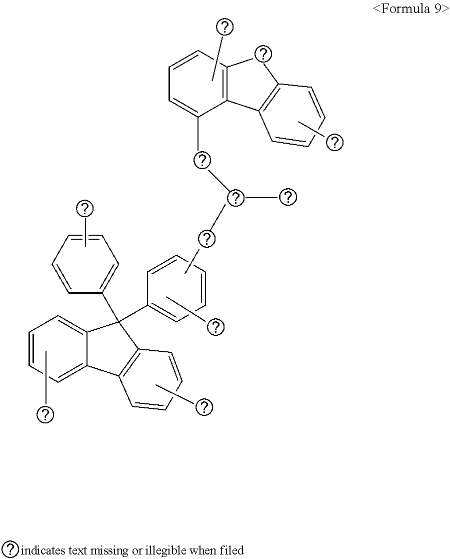
wherein, in Formulas 6 to 9,
z is an integer from 0 to 5, and
u, v, w, x, y, R²⁰ to R²⁵, L²⁰, L²¹, Ar²⁰, and X²⁰ are the same as those defined in claim 1.

6. The device of claim 1, wherein the first compound is represented by one of the following Formulas 10 and 11:
[Formulas 10 and 11] 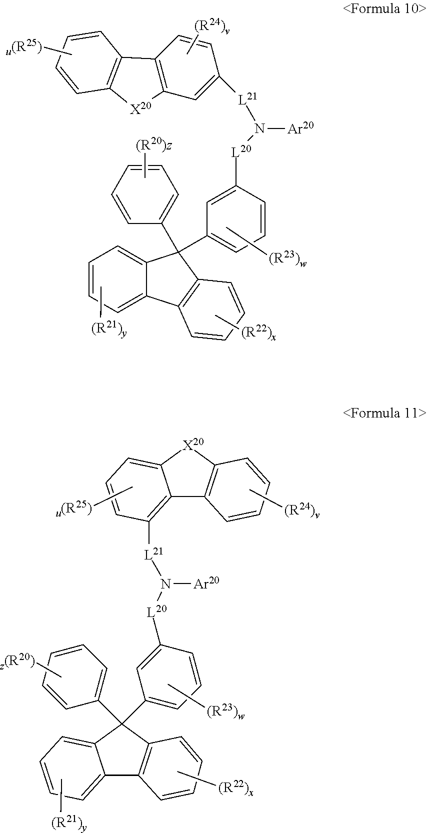
wherein, in Formulas 10 and 11, z is an integer from 0 to 5, and
u, v, w, x, y, R²⁰ to R²⁵, L²⁰, L²¹, Ar²⁰, and X²⁰ are the same as those defined in claim 1.

7. The device of claim 1, wherein the first compound is represented by one of the following Formulas 12 and 13:
[Formulas 12 and 13] 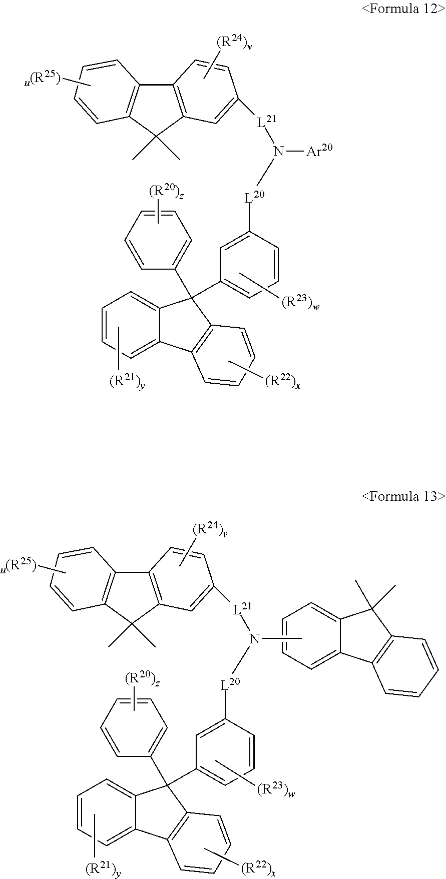
wherein, in Formulas 12 and 13,
z is an integer from 0 to 5, and
u, v, w, x, y, R²⁰ to R²⁵, L²⁰, L²¹, and Ar²⁰ are the same as those defined in claim 1.

8. The device of claim 1, wherein the first compound comprises one or more of the following compounds:

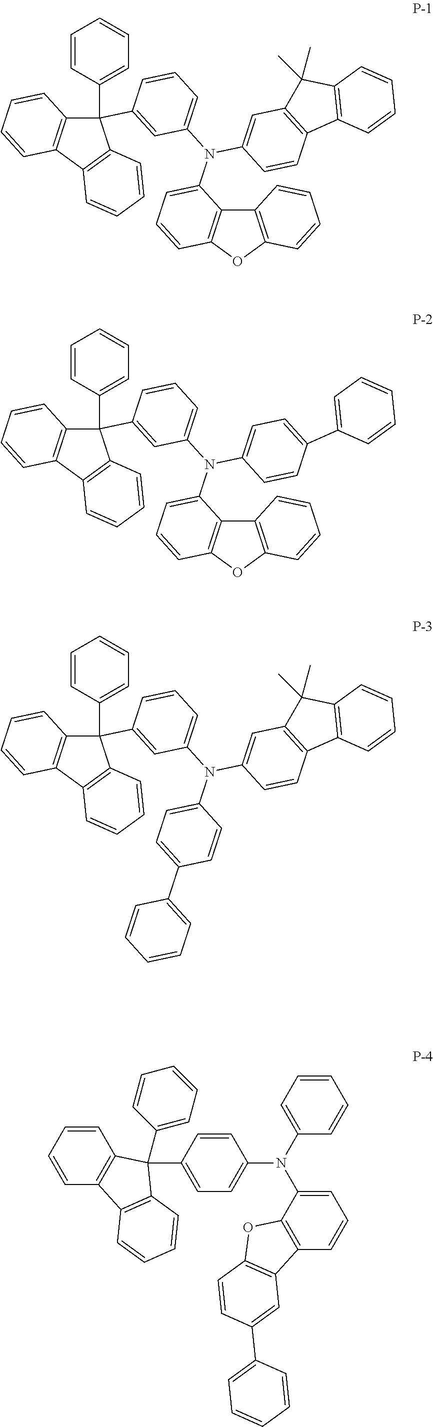 [SMILES: CC1(C)c2ccccc2-c2ccc(N(c3cccc(C4(c5ccccc5)c5ccccc5-c5ccccc54)c3)c3cccc4oc5ccccc5c34)cc21 ; c1ccc(-c2ccc(N(c3cccc(C4(c5ccccc5)c5ccccc5-c5ccccc54)c3)c3cccc4oc5ccccc5c34)cc2)cc1 ; CC1(C)c2ccccc2-c2ccc(N(c3ccc(-c4ccccc4)cc3)c3cccc(C4(c5ccccc5)c5ccccc5-c5ccccc54)c3)cc21 ; c1ccc(-c2ccc3oc4c(N(c5ccccc5)c5ccc(C6(c7ccccc7)c7ccccc7-c7ccccc76)cc5)cccc4c3c2)cc1]

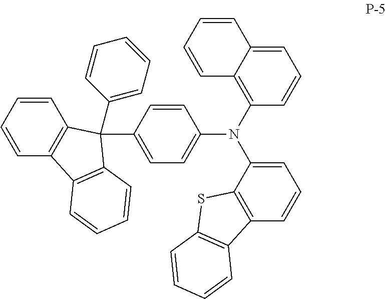 [SMILES: c1ccc(C2(c3ccc(N(c4cccc5ccccc45)c4cccc5c4sc4ccccc45)cc3)c3ccccc3-c3ccccc32)cc1]

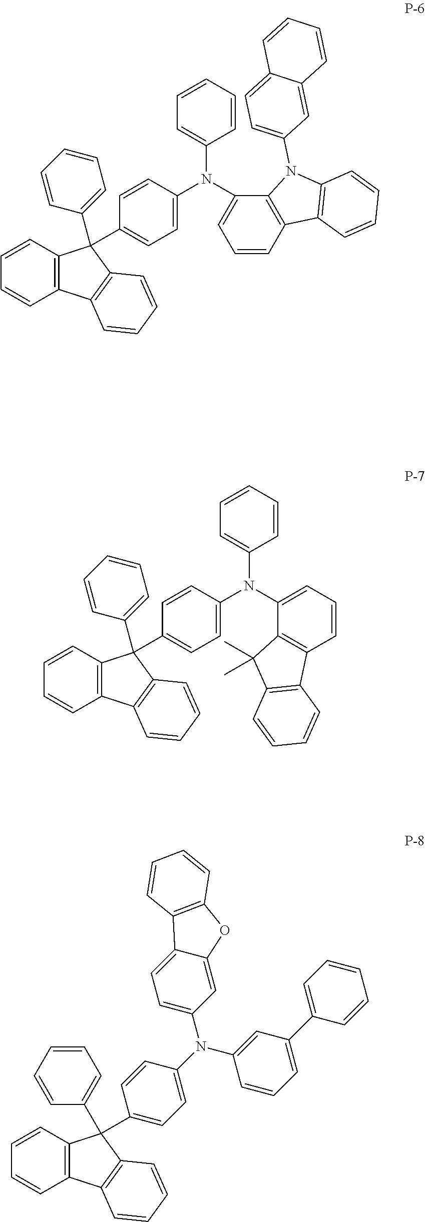 [SMILES: c1ccc(N(c2ccc(C3(c4ccccc4)c4ccccc4-c4ccccc43)cc2)c2cccc3c4ccccc4n(-c4ccc5ccccc5c4)c23)cc1 ; CC1(C)c2ccccc2-c2cccc(N(c3ccccc3)c3ccc(C4(c5ccccc5)c5ccccc5-c5ccccc54)cc3)c21 ; c1ccc(-c2cccc(N(c3ccc(C4(c5ccccc5)c5ccccc5-c5ccccc54)cc3)c3ccc4c(c3)oc3ccccc34)c2)cc1]

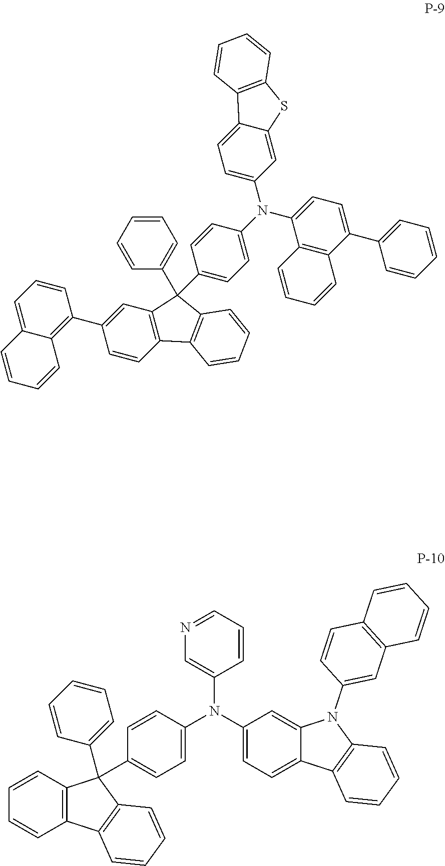 [SMILES: c1ccc(-c2ccc(N(c3ccc(C4(c5ccccc5)c5ccccc5-c5ccc(-c6cccc7ccccc67)cc54)cc3)c3ccc4c(c3)sc3ccccc34)c3ccccc23)cc1 ; c1ccc(C2(c3ccc(N(c4cccnc4)c4ccc5c6ccccc6n(-c6ccc7ccccc7c6)c5c4)cc3)c3ccccc3-c3ccccc32)cc1]

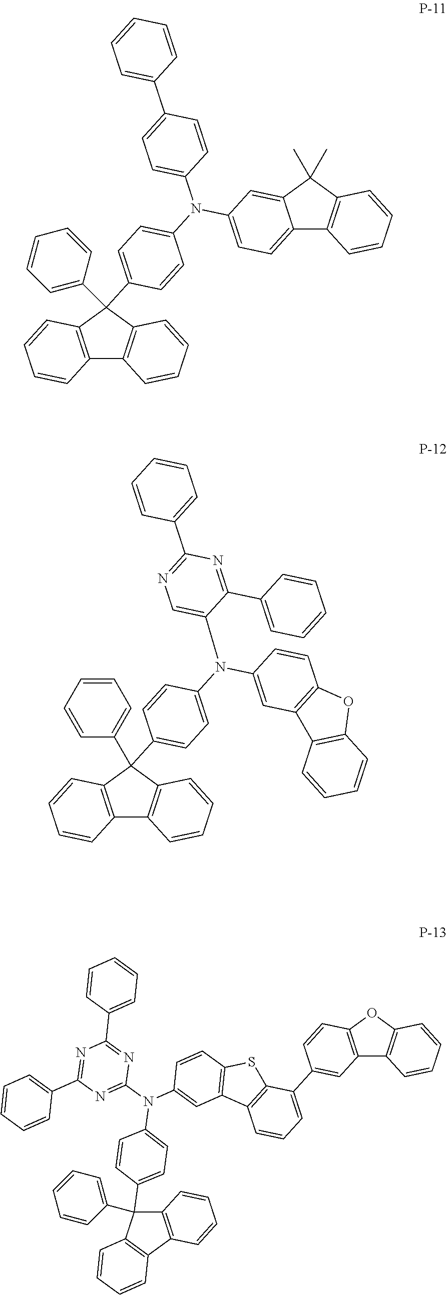 [SMILES: CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ncc(N(c3ccc(C4(c5ccccc5)c5ccccc5-c5ccccc54)cc3)c3ccc4oc5ccccc5c4c3)c(-c3ccccc3)n2)cc1 ; c1ccc(-c2nc(-c3ccccc3)nc(N(c3ccc(C4(c5ccccc5)c5ccccc5-c5ccccc54)cc3)c3ccc4sc5c(-c6ccc7oc8ccccc8c7c6)cccc5c4c3)n2)cc1]

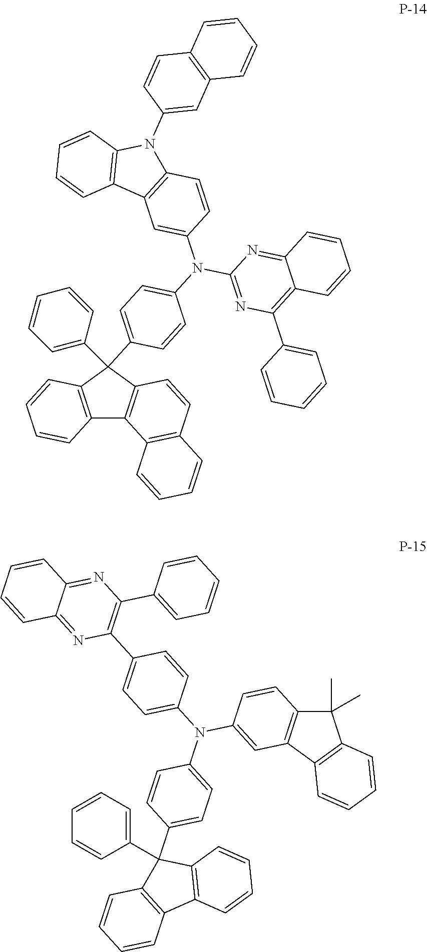 [SMILES: c1ccc(-c2nc(N(c3ccc(C4(c5ccccc5)c5ccccc5-c5c4ccc4ccccc54)cc3)c3ccc4c(c3)c3ccccc3n4-c3ccc4ccccc4c3)nc3ccccc23)cc1 ; CC1(C)c2ccccc2-c2cc(N(c3ccc(-c4nc5ccccc5nc4-c4ccccc4)cc3)c3ccc(C4(c5ccccc5)c5ccccc5-c5ccccc54)cc3)ccc21]

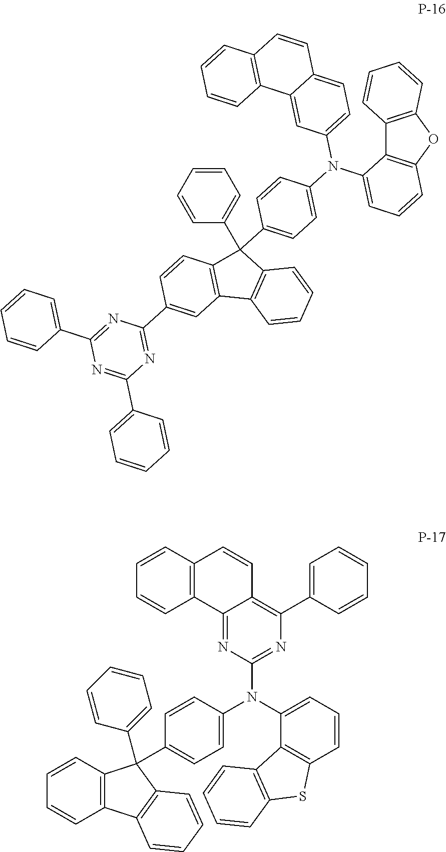 [SMILES: c1ccc(-c2nc(-c3ccccc3)nc(-c3ccc4c(c3)-c3ccccc3C4(c3ccccc3)c3ccc(N(c4ccc5ccc6ccccc6c5c4)c4cccc5oc6ccccc6c45)cc3)n2)cc1 ; c1ccc(-c2nc(N(c3ccc(C4(c5ccccc5)c5ccccc5-c5ccccc54)cc3)c3cccc4sc5ccccc5c34)nc3c2ccc2ccccc23)cc1]

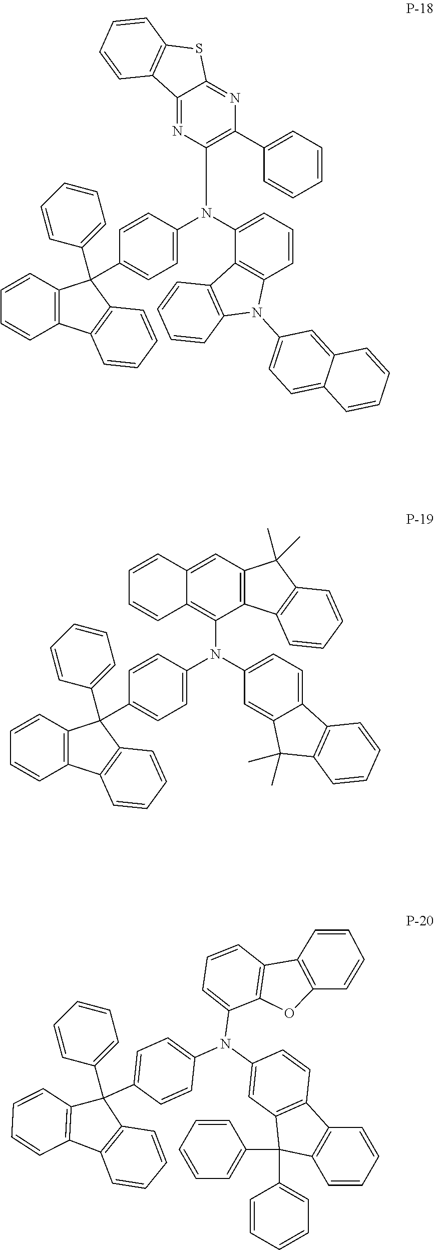 [SMILES: c1ccc(-c2nc3sc4ccccc4c3nc2N(c2ccc(C3(c4ccccc4)c4ccccc4-c4ccccc43)cc2)c2cccc3c2c2ccccc2n3-c2ccc3ccccc3c2)cc1 ; CC1(C)c2ccccc2-c2ccc(N(c3ccc(C4(c5ccccc5)c5ccccc5-c5ccccc54)cc3)c3c4c(cc5ccccc35)C(C)(C)c3ccccc3-4)cc21 ; c1ccc(C2(c3ccc(N(c4ccc5c(c4)C(c4ccccc4)(c4ccccc4)c4ccccc4-5)c4cccc5c4oc4ccccc45)cc3)c3ccccc3-c3ccccc32)cc1]

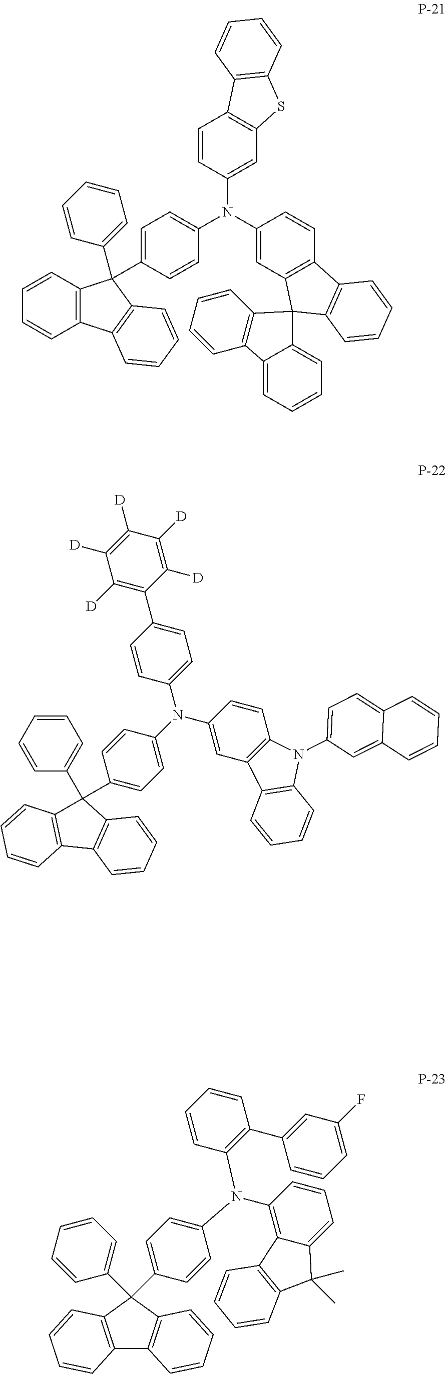 [SMILES: c1ccc(C2(c3ccc(N(c4ccc5c(c4)C4(c6ccccc6-c6ccccc64)c4ccccc4-5)c4ccc5c(c4)sc4ccccc45)cc3)c3ccccc3-c3ccccc32)cc1 ; [2H]c1c([2H])c([2H])c(-c2ccc(N(c3ccc(C4(c5ccccc5)c5ccccc5-c5ccccc54)cc3)c3ccc4c(c3)c3ccccc3n4-c3ccc4ccccc4c3)cc2)c([2H])c1[2H] ; CC1(C)c2ccccc2-c2c(N(c3ccc(C4(c5ccccc5)c5ccccc5-c5ccccc54)cc3)c3ccccc3-c3cccc(F)c3)cccc21]

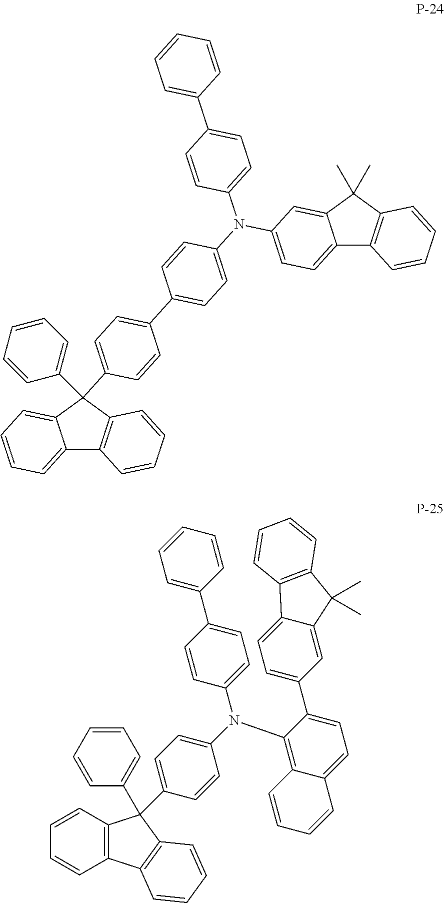 [SMILES: CC1(C)c2ccccc2-c2ccc(N(c3ccc(-c4ccccc4)cc3)c3ccc(-c4ccc(C5(c6ccccc6)c6ccccc6-c6ccccc65)cc4)cc3)cc21 ; CC1(C)c2ccccc2-c2ccc(-c3ccc4ccccc4c3N(c3ccc(-c4ccccc4)cc3)c3ccc(C4(c5ccccc5)c5ccccc5-c5ccccc54)cc3)cc21]

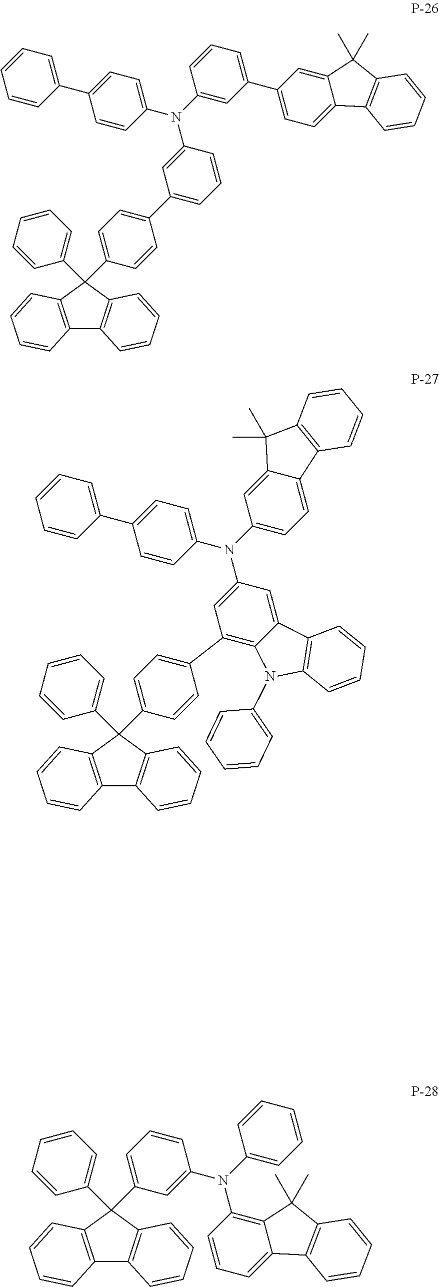 [SMILES: CC1(C)c2ccccc2-c2ccc(-c3cccc(N(c4ccc(-c5ccccc5)cc4)c4cccc(-c5ccc(C6(c7ccccc7)c7ccccc7-c7ccccc76)cc5)c4)c3)cc21 ; CC1(C)c2ccccc2-c2ccc(N(c3ccc(-c4ccccc4)cc3)c3cc(-c4ccc(C5(c6ccccc6)c6ccccc6-c6ccccc65)cc4)c4c(c3)c3ccccc3n4-c3ccccc3)cc21 ; CC1(C)c2ccccc2-c2cccc(N(c3ccccc3)c3cccc(C4(c5ccccc5)c5ccccc5-c5ccccc54)c3)c21]

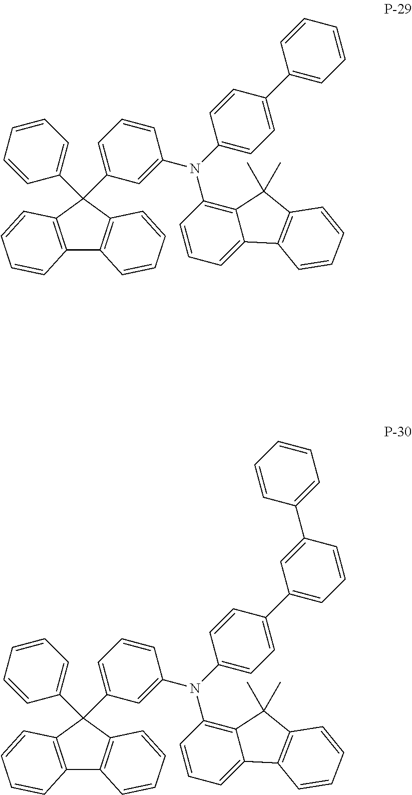 [SMILES: CC1(C)c2ccccc2-c2cccc(N(c3ccc(-c4ccccc4)cc3)c3cccc(C4(c5ccccc5)c5ccccc5-c5ccccc54)c3)c21 ; CC1(C)c2ccccc2-c2cccc(N(c3ccc(-c4cccc(-c5ccccc5)c4)cc3)c3cccc(C4(c5ccccc5)c5ccccc5-c5ccccc54)c3)c21]

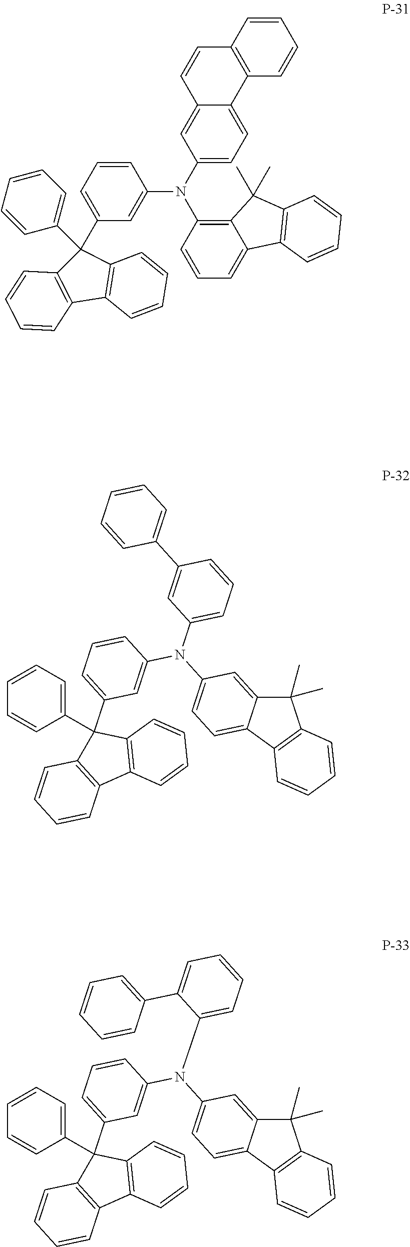 [SMILES: CC1(C)c2ccccc2-c2cccc(N(c3cccc(C4(c5ccccc5)c5ccccc5-c5ccccc54)c3)c3ccc4c(ccc5ccccc54)c3)c21 ; CC1(C)c2ccccc2-c2ccc(N(c3cccc(-c4ccccc4)c3)c3cccc(C4(c5ccccc5)c5ccccc5-c5ccccc54)c3)cc21 ; CC1(C)c2ccccc2-c2ccc(N(c3cccc(C4(c5ccccc5)c5ccccc5-c5ccccc54)c3)c3ccccc3-c3ccccc3)cc21]

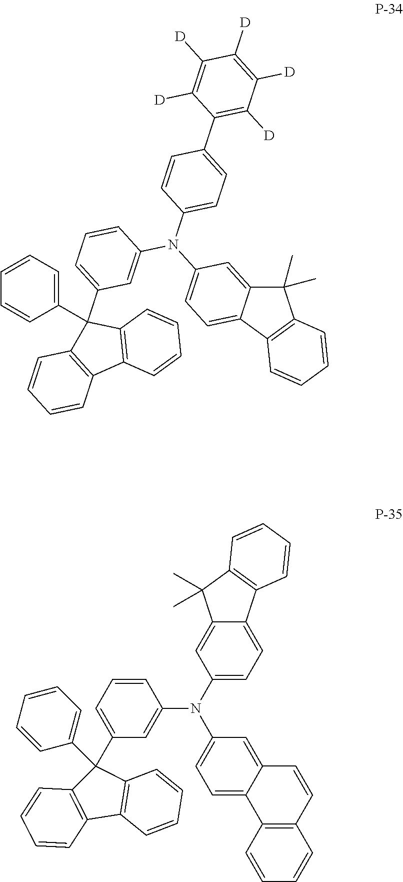 [SMILES: [2H]c1c([2H])c([2H])c(-c2ccc(N(c3cccc(C4(c5ccccc5)c5ccccc5-c5ccccc54)c3)c3ccc4c(c3)C(C)(C)c3ccccc3-4)cc2)c([2H])c1[2H] ; CC1(C)c2ccccc2-c2ccc(N(c3cccc(C4(c5ccccc5)c5ccccc5-c5ccccc54)c3)c3ccc4c(ccc5ccccc54)c3)cc21]

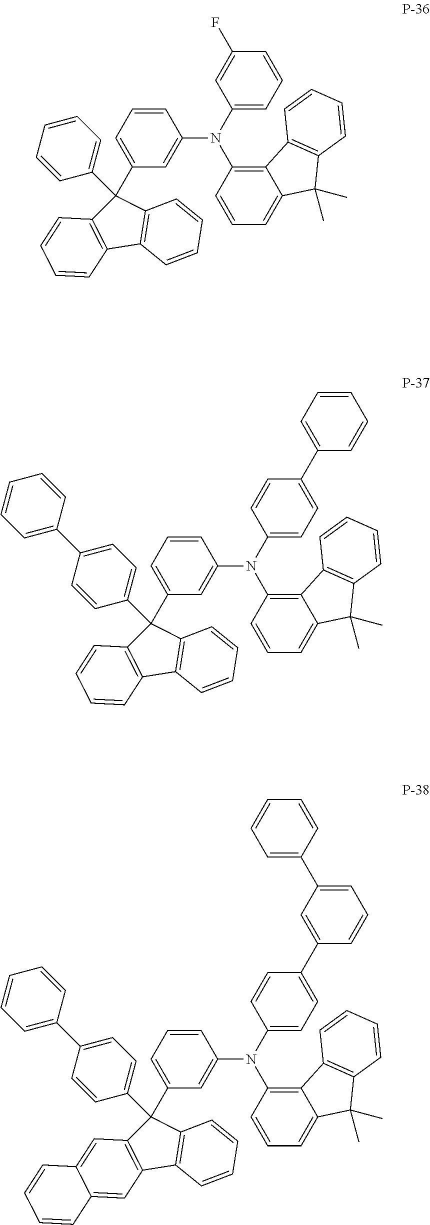 [SMILES: CC1(C)c2ccccc2-c2c(N(c3cccc(F)c3)c3cccc(C4(c5ccccc5)c5ccccc5-c5ccccc54)c3)cccc21 ; CC1(C)c2ccccc2-c2c(N(c3ccc(-c4ccccc4)cc3)c3cccc(C4(c5ccc(-c6ccccc6)cc5)c5ccccc5-c5ccccc54)c3)cccc21 ; CC1(C)c2ccccc2-c2c(N(c3ccc(-c4cccc(-c5ccccc5)c4)cc3)c3cccc(C4(c5ccc(-c6ccccc6)cc5)c5ccccc5-c5cc6ccccc6cc54)c3)cccc21]

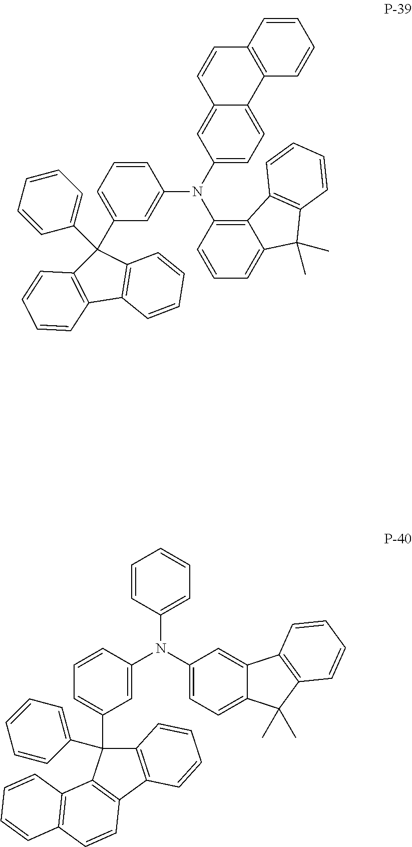 [SMILES: CC1(C)c2ccccc2-c2c(N(c3cccc(C4(c5ccccc5)c5ccccc5-c5ccccc54)c3)c3ccc4c(ccc5ccccc54)c3)cccc21 ; CC1(C)c2ccccc2-c2cc(N(c3ccccc3)c3cccc(C4(c5ccccc5)c5ccccc5-c5ccc6ccccc6c54)c3)ccc21]

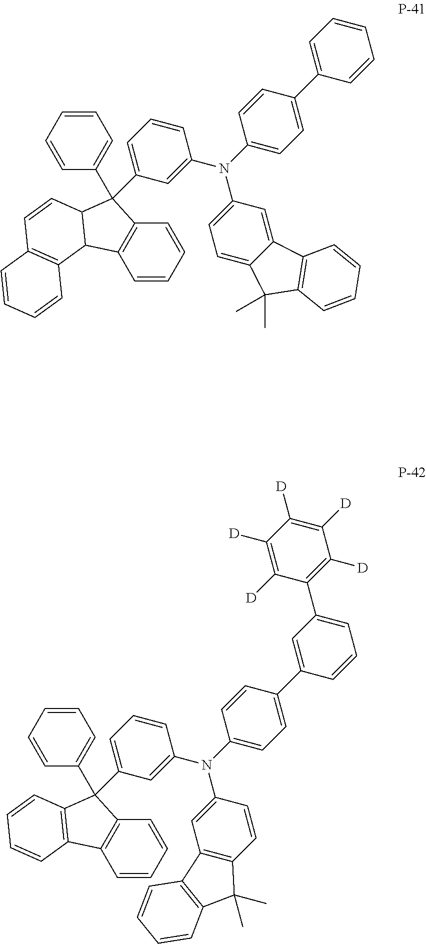 [SMILES: CC1(C)c2ccccc2-c2cc(N(c3ccc(-c4ccccc4)cc3)c3cccc(C4(c5ccccc5)c5ccccc5C5c6ccccc6C=CC54)c3)ccc21 ; [2H]c1c([2H])c([2H])c(-c2cccc(-c3ccc(N(c4cccc(C5(c6ccccc6)c6ccccc6-c6ccccc65)c4)c4ccc5c(c4)-c4ccccc4C5(C)C)cc3)c2)c([2H])c1[2H]]

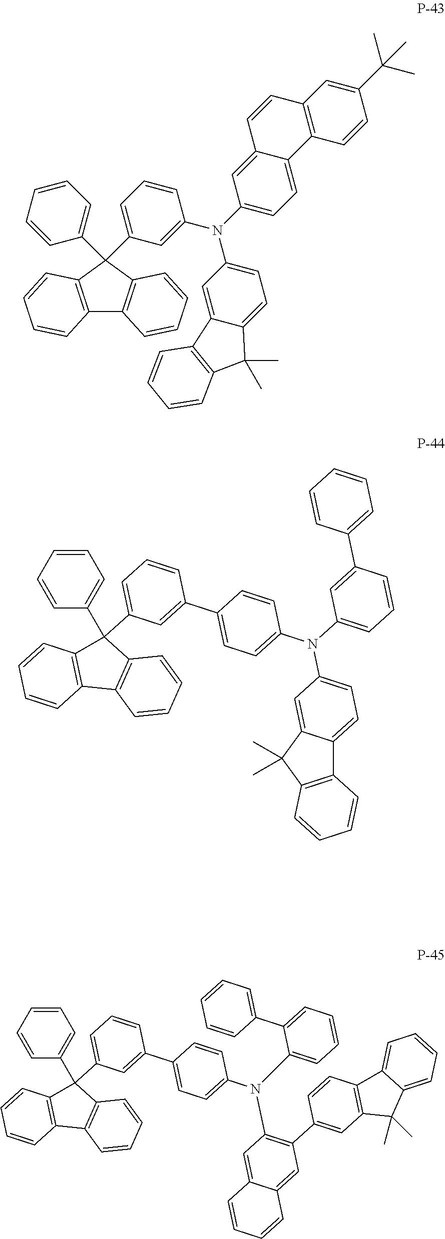 [SMILES: CC(C)(C)c1ccc2c(ccc3cc(N(c4cccc(C5(c6ccccc6)c6ccccc6-c6ccccc65)c4)c4ccc5c(c4)-c4ccccc4C5(C)C)ccc32)c1 ; CC1(C)c2ccccc2-c2ccc(N(c3ccc(-c4cccc(C5(c6ccccc6)c6ccccc6-c6ccccc65)c4)cc3)c3cccc(-c4ccccc4)c3)cc21 ; CC1(C)c2ccccc2-c2ccc(-c3cc4ccccc4cc3N(c3ccc(-c4cccc(C5(c6ccccc6)c6ccccc6-c6ccccc65)c4)cc3)c3ccccc3-c3ccccc3)cc21]

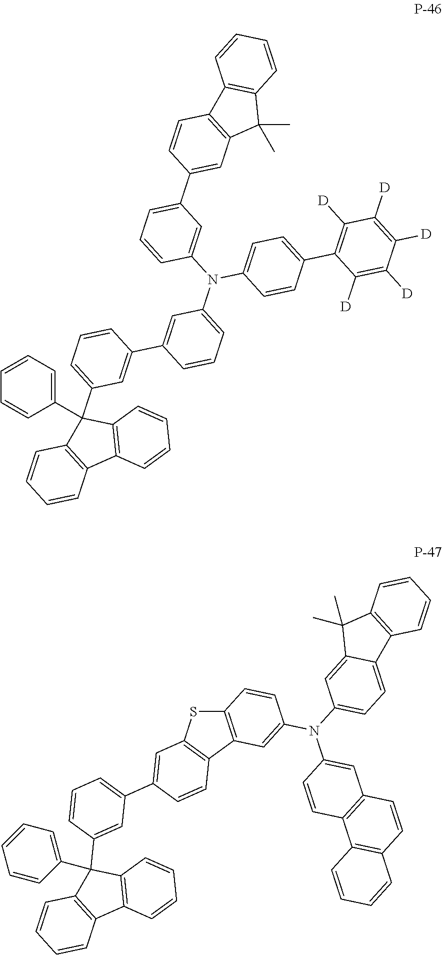 [SMILES: [2H]c1c([2H])c([2H])c(-c2ccc(N(c3cccc(-c4cccc(C5(c6ccccc6)c6ccccc6-c6ccccc65)c4)c3)c3cccc(-c4ccc5c(c4)C(C)(C)c4ccccc4-5)c3)cc2)c([2H])c1[2H] ; CC1(C)c2ccccc2-c2ccc(N(c3ccc4c(ccc5ccccc54)c3)c3ccc4sc5cc(-c6cccc(C7(c8ccccc8)c8ccccc8-c8ccccc87)c6)ccc5c4c3)cc21]

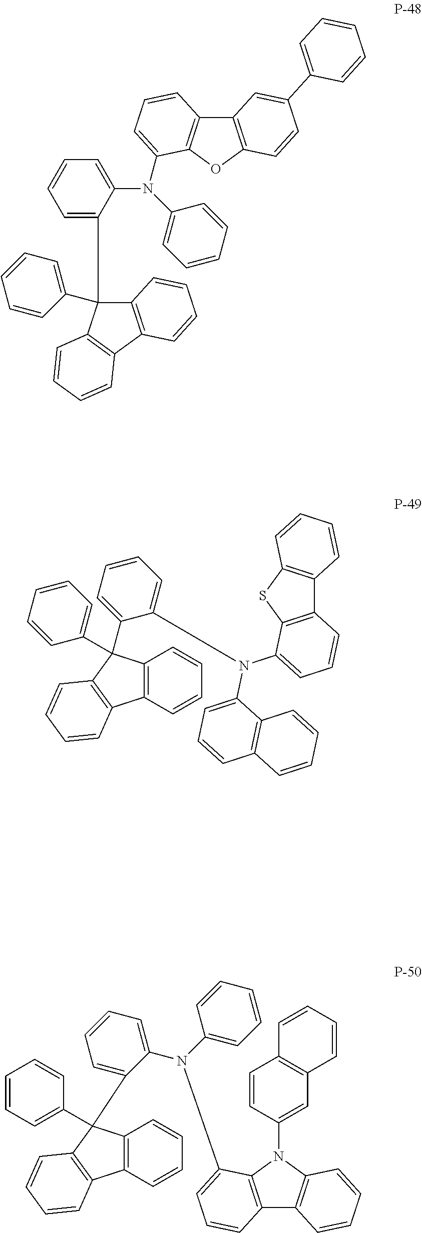 [SMILES: c1ccc(-c2ccc3oc4c(N(c5ccccc5)c5ccccc5C5(c6ccccc6)c6ccccc6-c6ccccc65)cccc4c3c2)cc1 ; c1ccc(C2(c3ccccc3N(c3cccc4ccccc34)c3cccc4c3sc3ccccc34)c3ccccc3-c3ccccc32)cc1 ; c1ccc(N(c2ccccc2C2(c3ccccc3)c3ccccc3-c3ccccc32)c2cccc3c4ccccc4n(-c4ccc5ccccc5c4)c23)cc1]

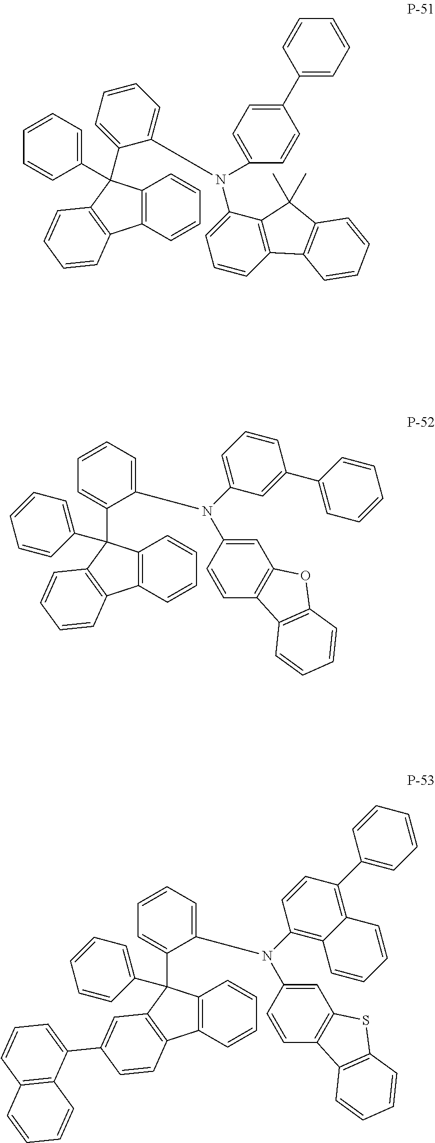 [SMILES: CC1(C)c2ccccc2-c2cccc(N(c3ccc(-c4ccccc4)cc3)c3ccccc3C3(c4ccccc4)c4ccccc4-c4ccccc43)c21 ; c1ccc(-c2cccc(N(c3ccc4c(c3)oc3ccccc34)c3ccccc3C3(c4ccccc4)c4ccccc4-c4ccccc43)c2)cc1 ; c1ccc(-c2ccc(N(c3ccc4c(c3)sc3ccccc34)c3ccccc3C3(c4ccccc4)c4ccccc4-c4ccc(-c5cccc6ccccc56)cc43)c3ccccc23)cc1]

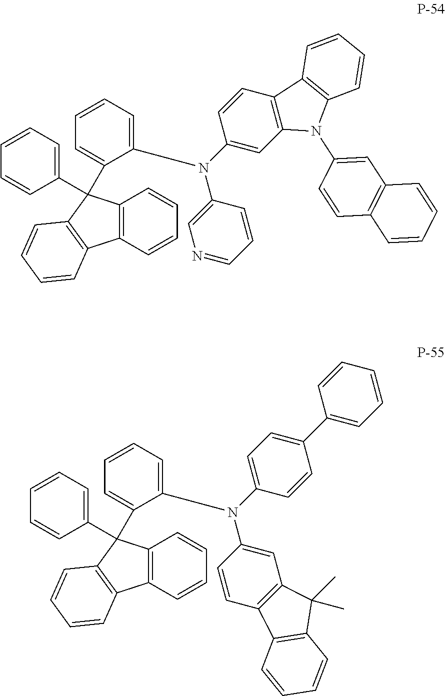 [SMILES: c1ccc(C2(c3ccccc3N(c3cccnc3)c3ccc4c5ccccc5n(-c5ccc6ccccc6c5)c4c3)c3ccccc3-c3ccccc32)cc1 ; CC1(C)c2ccccc2-c2ccc(N(c3ccc(-c4ccccc4)cc3)c3ccccc3C3(c4ccccc4)c4ccccc4-c4ccccc43)cc21]

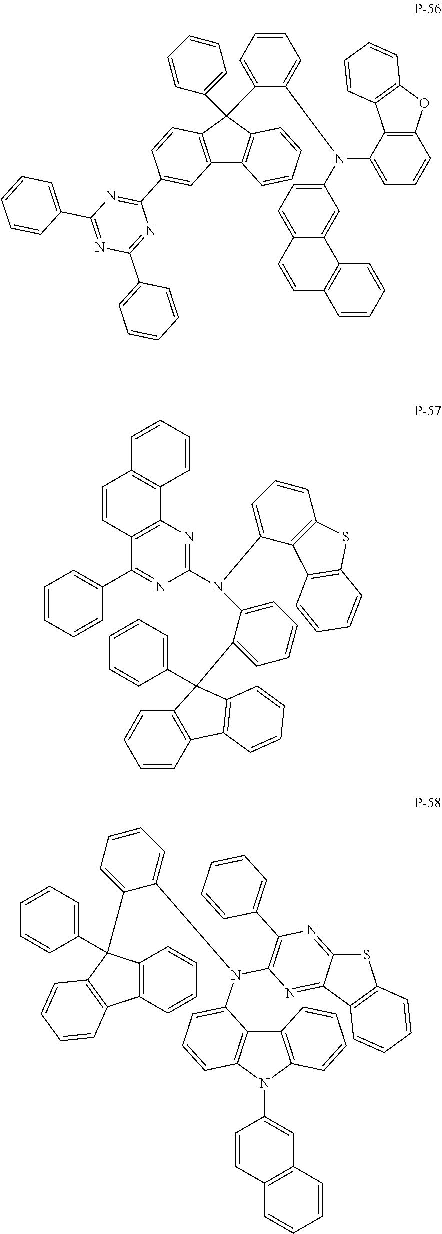 [SMILES: c1ccc(-c2nc(-c3ccccc3)nc(-c3ccc4c(c3)-c3ccccc3C4(c3ccccc3)c3ccccc3N(c3ccc4ccc5ccccc5c4c3)c3cccc4oc5ccccc5c34)n2)cc1 ; c1ccc(-c2nc(N(c3ccccc3C3(c4ccccc4)c4ccccc4-c4ccccc43)c3cccc4sc5ccccc5c34)nc3c2ccc2ccccc23)cc1 ; c1ccc(-c2nc3sc4ccccc4c3nc2N(c2ccccc2C2(c3ccccc3)c3ccccc3-c3ccccc32)c2cccc3c2c2ccccc2n3-c2ccc3ccccc3c2)cc1]

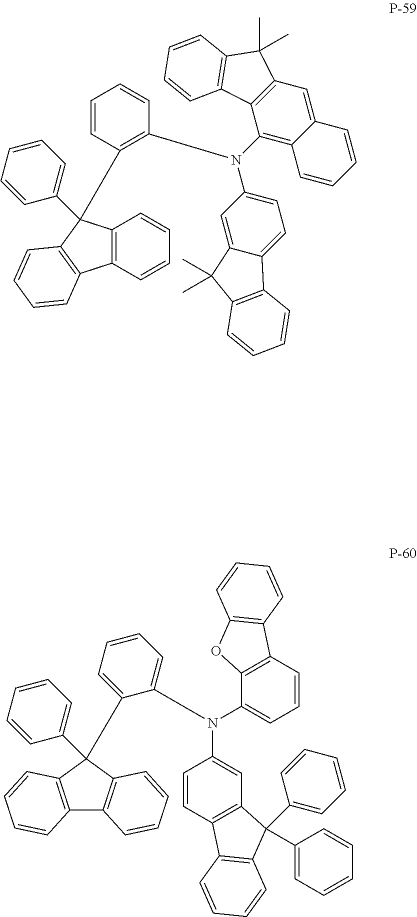 [SMILES: CC1(C)c2ccccc2-c2ccc(N(c3ccccc3C3(c4ccccc4)c4ccccc4-c4ccccc43)c3c4c(cc5ccccc35)C(C)(C)c3ccccc3-4)cc21 ; c1ccc(C2(c3ccccc3)c3ccccc3-c3ccc(N(c4ccccc4C4(c5ccccc5)c5ccccc5-c5ccccc54)c4cccc5c4oc4ccccc45)cc32)cc1]

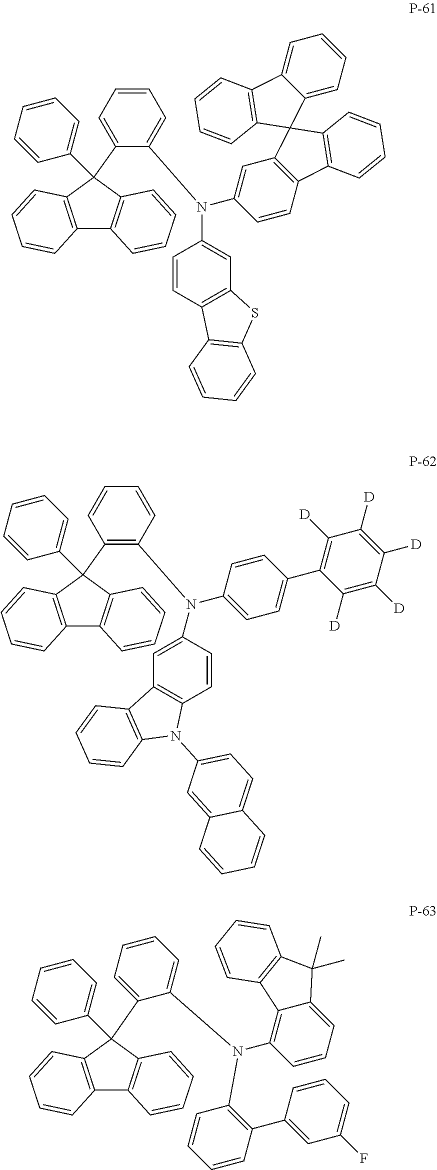 [SMILES: c1ccc(C2(c3ccccc3N(c3ccc4c(c3)C3(c5ccccc5-c5ccccc53)c3ccccc3-4)c3ccc4c(c3)sc3ccccc34)c3ccccc3-c3ccccc32)cc1 ; [2H]c1c([2H])c([2H])c(-c2ccc(N(c3ccc4c(c3)c3ccccc3n4-c3ccc4ccccc4c3)c3ccccc3C3(c4ccccc4)c4ccccc4-c4ccccc43)cc2)c([2H])c1[2H] ; CC1(C)c2ccccc2-c2c(N(c3ccccc3-c3cccc(F)c3)c3ccccc3C3(c4ccccc4)c4ccccc4-c4ccccc43)cccc21]

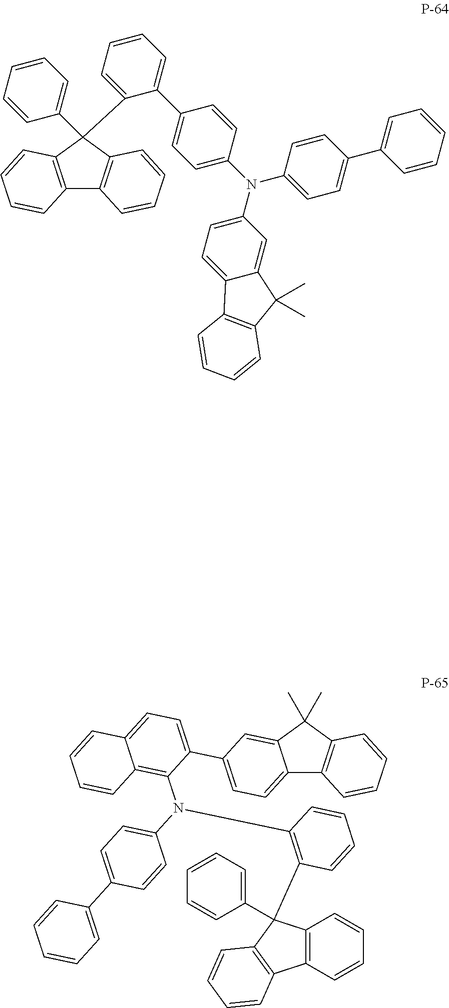 [SMILES: CC1(C)c2ccccc2-c2ccc(N(c3ccc(-c4ccccc4)cc3)c3ccc(-c4ccccc4C4(c5ccccc5)c5ccccc5-c5ccccc54)cc3)cc21 ; CC1(C)c2ccccc2-c2ccc(-c3ccc4ccccc4c3N(c3ccc(-c4ccccc4)cc3)c3ccccc3C3(c4ccccc4)c4ccccc4-c4ccccc43)cc21]

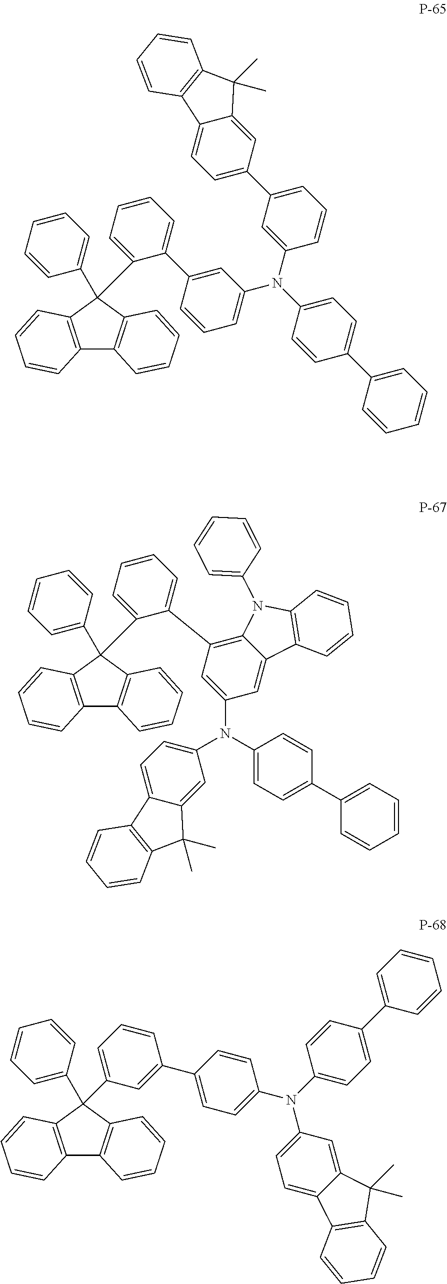 [SMILES: CC1(C)c2ccccc2-c2ccc(-c3cccc(N(c4ccc(-c5ccccc5)cc4)c4cccc(-c5ccccc5C5(c6ccccc6)c6ccccc6-c6ccccc65)c4)c3)cc21 ; CC1(C)c2ccccc2-c2ccc(N(c3ccc(-c4ccccc4)cc3)c3cc(-c4ccccc4C4(c5ccccc5)c5ccccc5-c5ccccc54)c4c(c3)c3ccccc3n4-c3ccccc3)cc21 ; CC1(C)c2ccccc2-c2ccc(N(c3ccc(-c4ccccc4)cc3)c3ccc(-c4cccc(C5(c6ccccc6)c6ccccc6-c6ccccc65)c4)cc3)cc21]

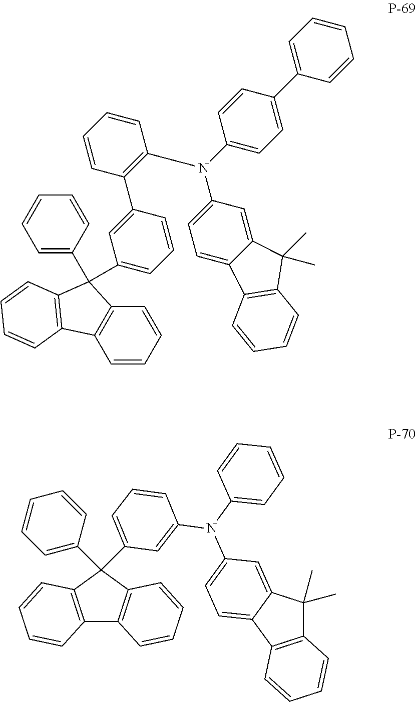 [SMILES: CC1(C)c2ccccc2-c2ccc(N(c3ccc(-c4ccccc4)cc3)c3ccccc3-c3cccc(C4(c5ccccc5)c5ccccc5-c5ccccc54)c3)cc21 ; CC1(C)c2ccccc2-c2ccc(N(c3ccccc3)c3cccc(C4(c5ccccc5)c5ccccc5-c5ccccc54)c3)cc21]

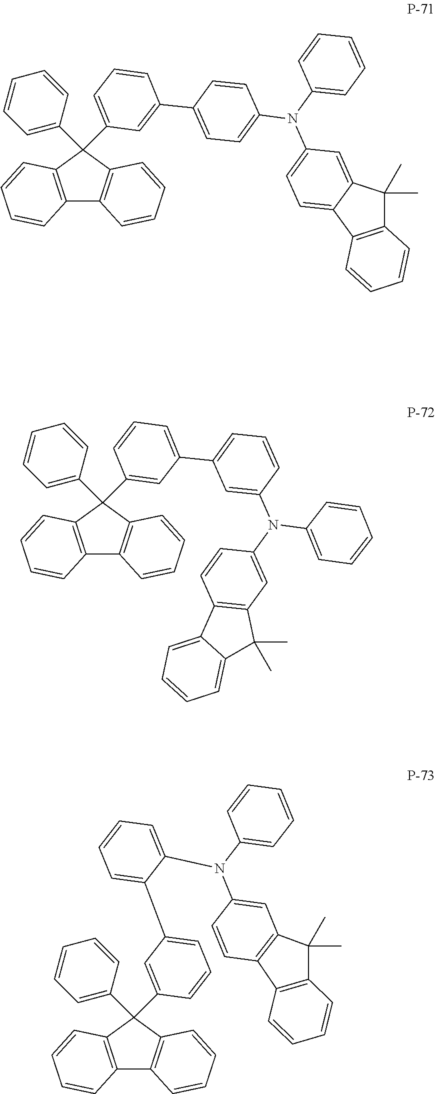 [SMILES: CC1(C)c2ccccc2-c2ccc(N(c3ccccc3)c3ccc(-c4cccc(C5(c6ccccc6)c6ccccc6-c6ccccc65)c4)cc3)cc21 ; CC1(C)c2ccccc2-c2ccc(N(c3ccccc3)c3cccc(-c4cccc(C5(c6ccccc6)c6ccccc6-c6ccccc65)c4)c3)cc21 ; CC1(C)c2ccccc2-c2ccc(N(c3ccccc3)c3ccccc3-c3cccc(C4(c5ccccc5)c5ccccc5-c5ccccc54)c3)cc21]

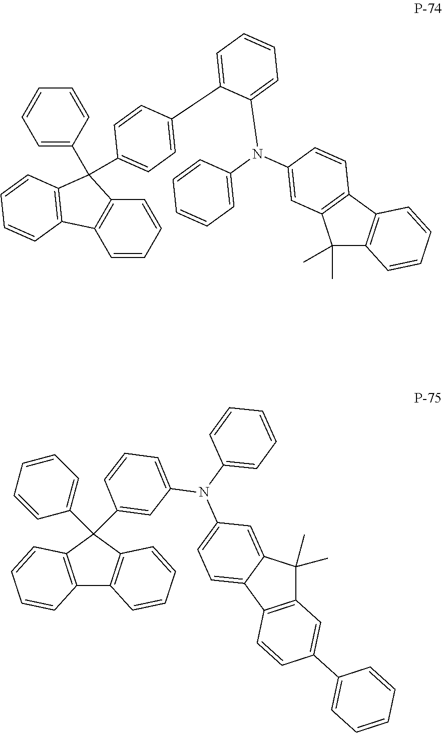 [SMILES: CC1(C)c2ccccc2-c2ccc(N(c3ccccc3)c3ccccc3-c3ccc(C4(c5ccccc5)c5ccccc5-c5ccccc54)cc3)cc21 ; CC1(C)c2cc(-c3ccccc3)ccc2-c2ccc(N(c3ccccc3)c3cccc(C4(c5ccccc5)c5ccccc5-c5ccccc54)c3)cc21]

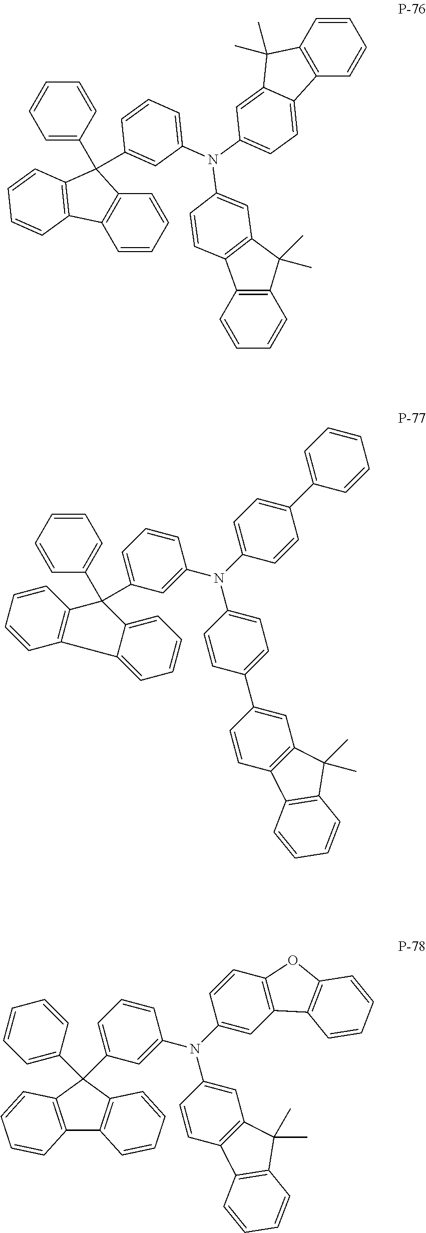 [SMILES: CC1(C)c2ccccc2-c2ccc(N(c3cccc(C4(c5ccccc5)c5ccccc5-c5ccccc54)c3)c3ccc4c(c3)C(C)(C)c3ccccc3-4)cc21 ; CC1(C)c2ccccc2-c2ccc(-c3ccc(N(c4ccc(-c5ccccc5)cc4)c4cccc(C5(c6ccccc6)c6ccccc6-c6ccccc65)c4)cc3)cc21 ; CC1(C)c2ccccc2-c2ccc(N(c3cccc(C4(c5ccccc5)c5ccccc5-c5ccccc54)c3)c3ccc4oc5ccccc5c4c3)cc21]

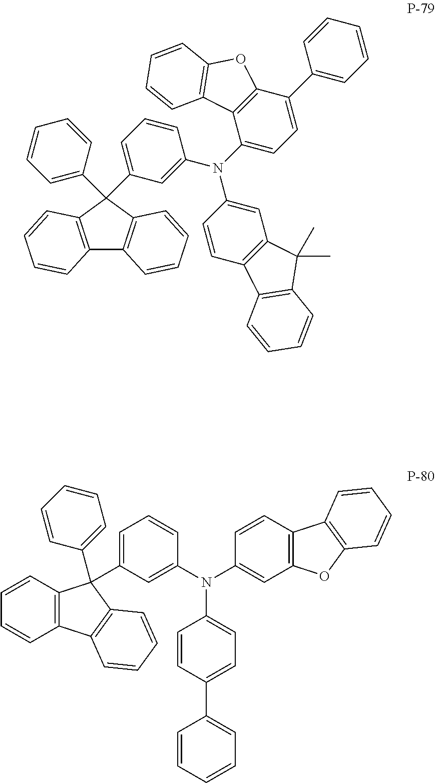 [SMILES: CC1(C)c2ccccc2-c2ccc(N(c3cccc(C4(c5ccccc5)c5ccccc5-c5ccccc54)c3)c3ccc(-c4ccccc4)c4oc5ccccc5c34)cc21 ; c1ccc(-c2ccc(N(c3cccc(C4(c5ccccc5)c5ccccc5-c5ccccc54)c3)c3ccc4c(c3)oc3ccccc34)cc2)cc1]

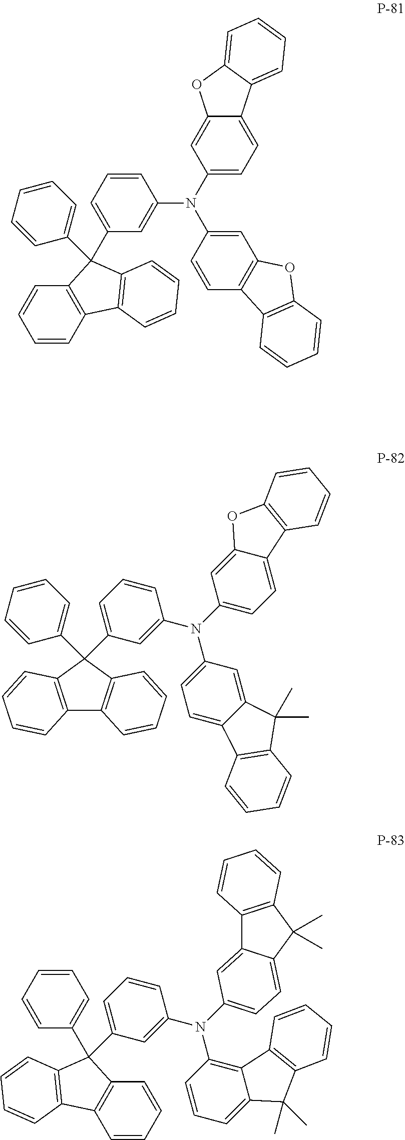

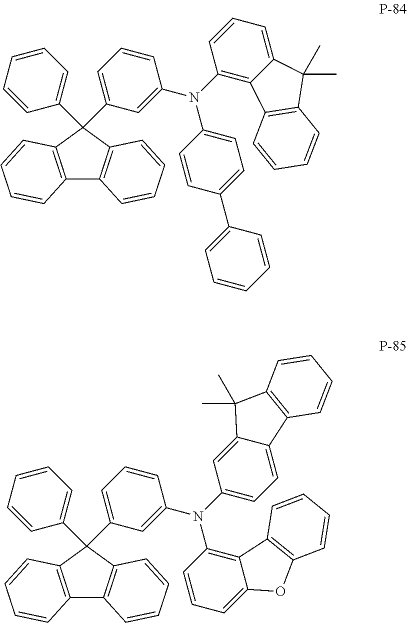 [SMILES: CC1(C)c2ccccc2-c2c(N(c3ccc(-c4ccccc4)cc3)c3cccc(C4(c5ccccc5)c5ccccc5-c5ccccc54)c3)cccc21 ; CC1(C)c2ccccc2-c2ccc(N(c3cccc(C4(c5ccccc5)c5ccccc5-c5ccccc54)c3)c3cccc4oc5ccccc5c34)cc21]

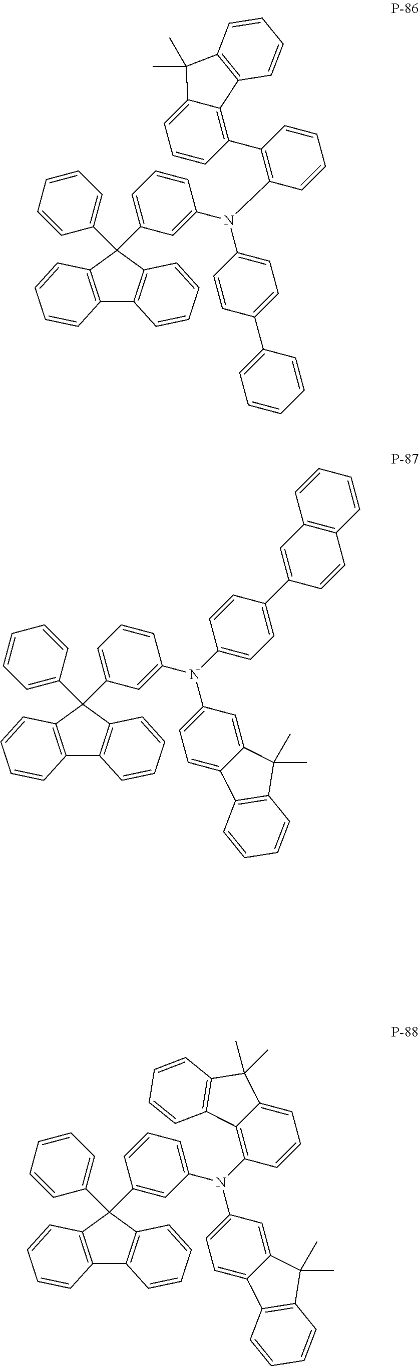 [SMILES: CC1(C)c2ccccc2-c2c(-c3ccccc3N(c3ccc(-c4ccccc4)cc3)c3cccc(C4(c5ccccc5)c5ccccc5-c5ccccc54)c3)cccc21 ; CC1(C)c2ccccc2-c2ccc(N(c3ccc(-c4ccc5ccccc5c4)cc3)c3cccc(C4(c5ccccc5)c5ccccc5-c5ccccc54)c3)cc21 ; CC1(C)c2ccccc2-c2ccc(N(c3cccc(C4(c5ccccc5)c5ccccc5-c5ccccc54)c3)c3cccc4c3-c3ccccc3C4(C)C)cc21]

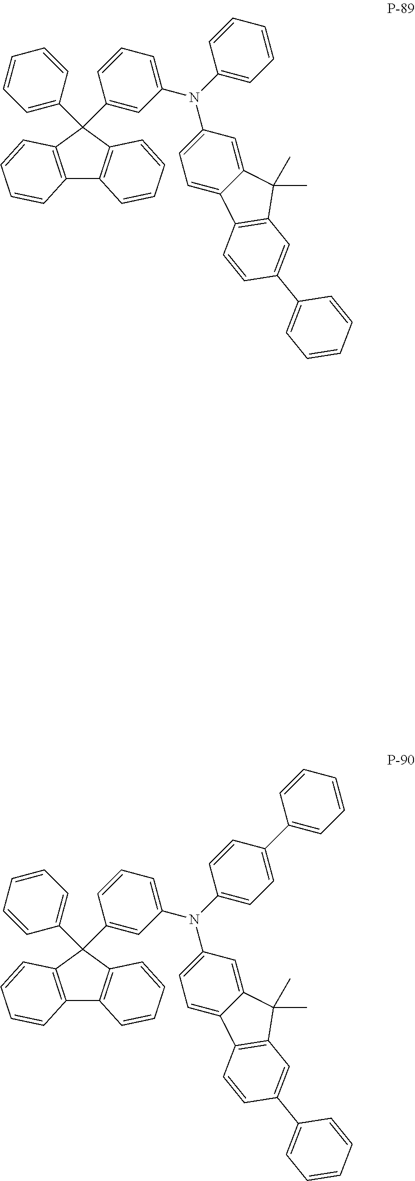 [SMILES: CC1(C)c2cc(-c3ccccc3)ccc2-c2ccc(N(c3ccccc3)c3cccc(C4(c5ccccc5)c5ccccc5-c5ccccc54)c3)cc21 ; CC1(C)c2cc(-c3ccccc3)ccc2-c2ccc(N(c3ccc(-c4ccccc4)cc3)c3cccc(C4(c5ccccc5)c5ccccc5-c5ccccc54)c3)cc21]

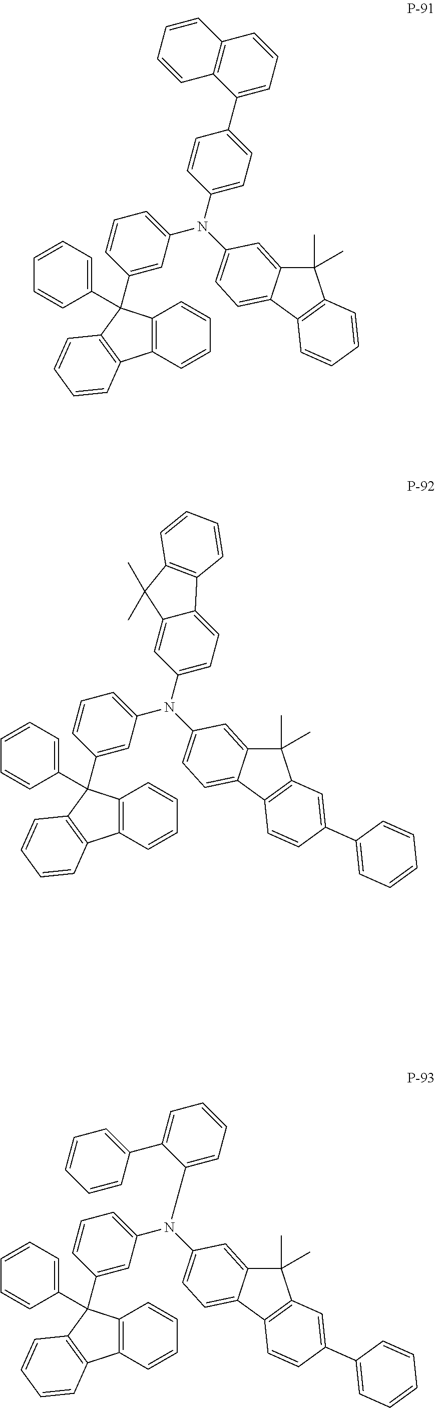 [SMILES: CC1(C)c2ccccc2-c2ccc(N(c3ccc(-c4cccc5ccccc45)cc3)c3cccc(C4(c5ccccc5)c5ccccc5-c5ccccc54)c3)cc21 ; CC1(C)c2ccccc2-c2ccc(N(c3cccc(C4(c5ccccc5)c5ccccc5-c5ccccc54)c3)c3ccc4c(c3)C(C)(C)c3cc(-c5ccccc5)ccc3-4)cc21 ; CC1(C)c2cc(-c3ccccc3)ccc2-c2ccc(N(c3cccc(C4(c5ccccc5)c5ccccc5-c5ccccc54)c3)c3ccccc3-c3ccccc3)cc21]

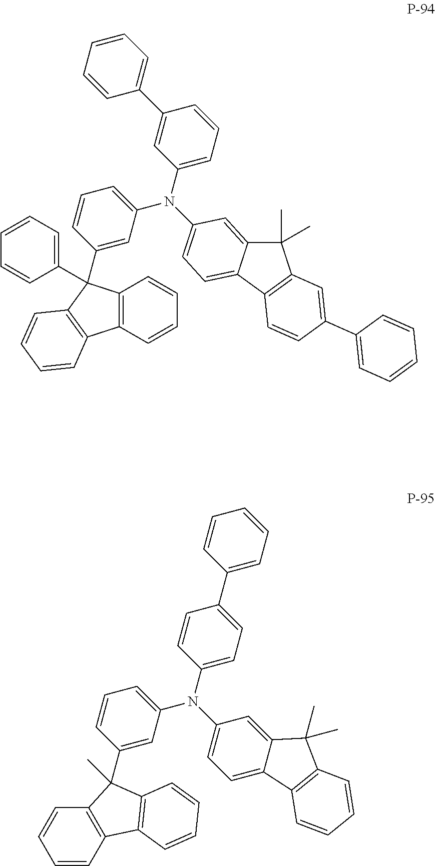 [SMILES: CC1(C)c2cc(-c3ccccc3)ccc2-c2ccc(N(c3cccc(-c4ccccc4)c3)c3cccc(C4(c5ccccc5)c5ccccc5-c5ccccc54)c3)cc21 ; CC1(C)c2ccccc2-c2ccc(N(c3ccc(-c4ccccc4)cc3)c3cccc(C4(C)c5ccccc5-c5ccccc54)c3)cc21]

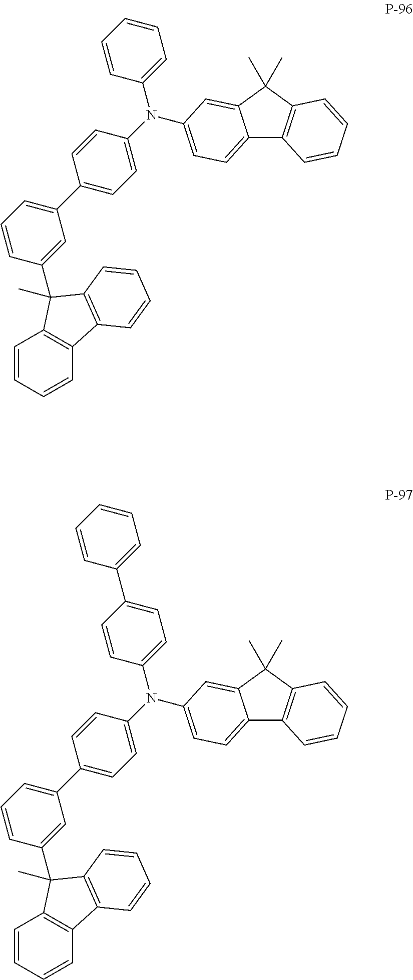 [SMILES: CC1(C)c2ccccc2-c2ccc(N(c3ccccc3)c3ccc(-c4cccc(C5(C)c6ccccc6-c6ccccc65)c4)cc3)cc21 ; CC1(C)c2ccccc2-c2ccc(N(c3ccc(-c4ccccc4)cc3)c3ccc(-c4cccc(C5(C)c6ccccc6-c6ccccc65)c4)cc3)cc21]

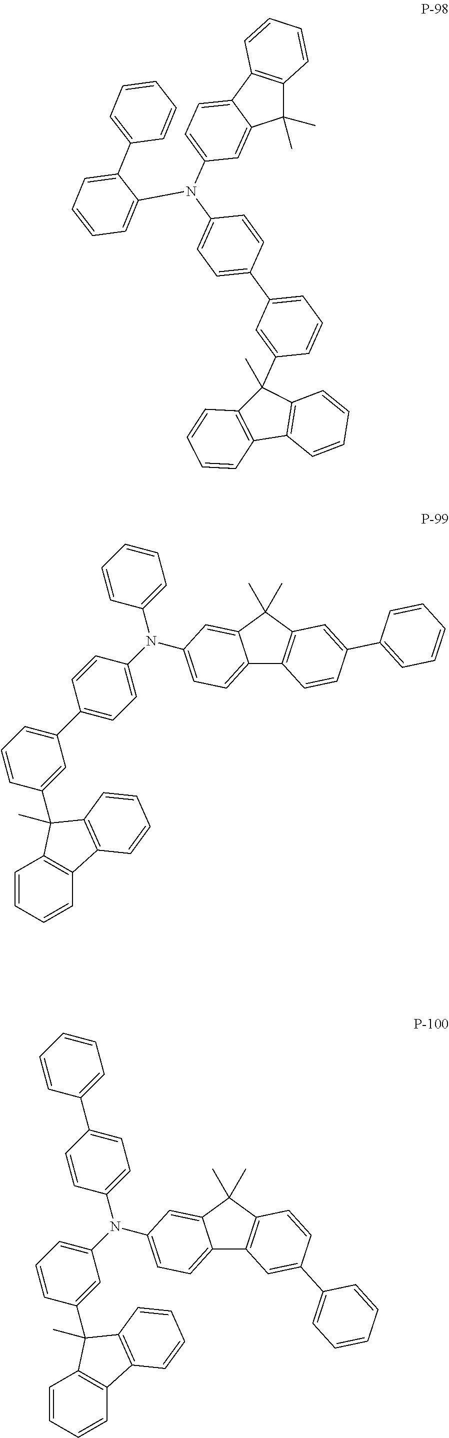 [SMILES: CC1(C)c2ccccc2-c2ccc(N(c3ccc(-c4cccc(C5(C)c6ccccc6-c6ccccc65)c4)cc3)c3ccccc3-c3ccccc3)cc21 ; CC1(C)c2cc(-c3ccccc3)ccc2-c2ccc(N(c3ccccc3)c3ccc(-c4cccc(C5(C)c6ccccc6-c6ccccc65)c4)cc3)cc21 ; CC1(C)c2ccc(-c3ccccc3)cc2-c2ccc(N(c3ccc(-c4ccccc4)cc3)c3cccc(C4(C)c5ccccc5-c5ccccc54)c3)cc21]

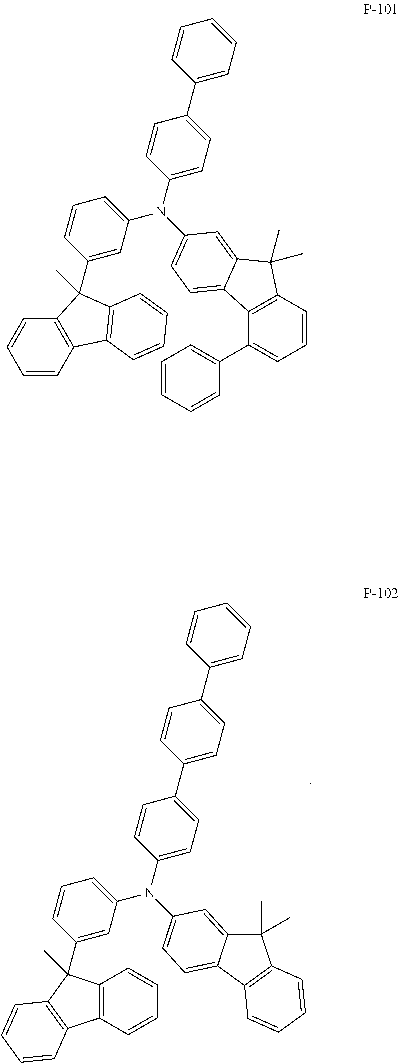 [SMILES: CC1(C)c2cc(N(c3ccc(-c4ccccc4)cc3)c3cccc(C4(C)c5ccccc5-c5ccccc54)c3)ccc2-c2c(-c3ccccc3)cccc21 ; CC1(C)c2ccccc2-c2ccc(N(c3ccc(-c4ccc(-c5ccccc5)cc4)cc3)c3cccc(C4(C)c5ccccc5-c5ccccc54)c3)cc21]

9. The device of claim 1, wherein the first doping material is represented by the following Formula E: 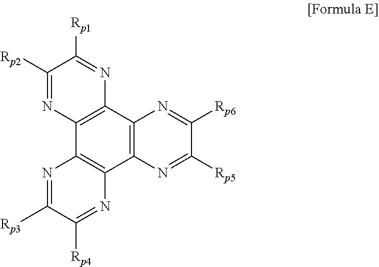
in Formula E, each of Rₚ₁ to Rₚ₆ is independently selected from the group consisting of hydrogen; a halogen group; a nitrile group; a nitro group; —SO₂R; —SOR; —SO₂NR₂; —SO₃R; a trifluoromethyl group; —COOR; —CONHR; —CONRR′; a C₁-C₃₀ alkoxyl group; a C₁-C₃₀ alkyl group; a C₂-C₂₀ alkenyl group; a C₂-C₃₀ heterocyclic group including at least one hetero atom of O, N, S, Si, or P; a fluorenyl group; a C₆-C₃₀ aryl group; a fused ring group of a C₃-C₃₀ aliphatic ring and C₆-C₃₀ aromatic ring; or —NRR′,
R and R′ are respectively selected from the group consisting of a C₁-C₃₀ alkyl group; a fluorenyl group; a C₆-C₃₀ aryl group; a fused ring group of a C₃-C₃₀ aliphatic ring and a C₆-C₃₀ aromatic ring; or a C₂-C₃₀ heterocyclic group including at least one hetero atom of O, N, S, Si, or P, and
in Formula E, each of the aryl group, the fluorenyl group, the heterocyclic group, the fused ring group, the alkyl group, the alkenyl group, the alkynyl group, and the alkoxyl group is substituted with one or more substituents selected from the group consisting of deuterium; a halogen group; a C₁-C₂₀ alkoxyl group; a C₁-C₂₀ alkyl group; a C₂-C₂₀ alkenyl group; a C₂-C₂₀ alkynyl group; a C₆-C₂₅ aryl group; a C₆-C₂₅ aryl group substituted with deuterium; a fluorenyl group; a C₂-C₂₀ heterocyclic group; or a C₃-C₂₀ cycloalkyl group.

10. The device of claim 1, wherein the first hole transport layer comprises a first doping material-doped layer doped with the first doping material and a first doping material undoped layer not doped with the first doping material, and
the first doping material-doped layer comprises the first compound and 5 to 15 parts by weight of the first doping material with respect to 100 parts by weight of the first compound.

11. The device of claim 1, wherein the second stack comprises a second hole transport region, a second emission layer, and a second electron transport region,
the second hole transport region comprises a second hole transport layer and a second auxiliary emission layer,
the second hole transport layer or the second auxiliary emission layer comprises a second compound represented by Formula 1,
the thickness of the second hole transport layer ranges from 250 Å to 700 Å,
10% to 50% of the thickness of the second hole transport layer is doped with a second doping material,
the third stack comprises a third hole transport region, a third emission layer, and a third electron transport region,
the third hole transport region comprises a third hole transport layer and a third auxiliary emission layer,
the third hole transport layer or the third auxiliary emission layer comprises a third compound represented by Formula 1,
the thickness of the third hole transport layer ranges from 250 Å to 700 Å, and
10% to 50% of the thickness of the third hole transport layer is doped with a third doping material.

12. The device of claim 11, wherein the first compound, the second compound, and the third compound are the same compounds.

13. The device of claim 1, wherein the first hole transport layer or the first auxiliary emission layer comprises at least one of the first compound or a fourth compound,
the fourth compound comprises a radical of a compound represented by the following Formula A or Formula B and is represented by at least one of the following Formula C or Formula D,
the thickness of the second hole transport layer ranges from 250 Å to 700 Å,
10% to 50% of the thickness of the second hole transport layer is doped with a second doping material,
[Formulas A, B, C, and D] 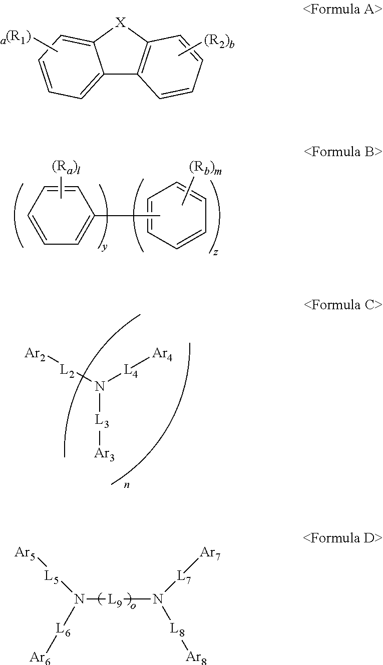
wherein, in Formula A,
1) each of a and b is independently an integer from 0 to 4,
2) X is O, S, CR′R″, or N-L₁-Ar₁,
3) R₁ and R₂ are respectively and independently selected from the group consisting of deuterium; tritium; a halogen; a cyano group; a nitro group; a C₆-C₆₀ aryl group; a fluorenyl group; a C₂-C₆₀ heterocyclic group including at least one hetero atom of O, N, S, Si, or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; or a C₆-C₃₀ aryloxy group, and the plurality of R₁s and the plurality of R₂s are bonded to form rings, respectively,
4) R′ and R″ are respectively and independently selected from the group consisting of hydrogen; deuterium; a C₅-C₆₀ aryl group; a fluorenyl group; a C₂-C₆₀ heterocyclic group including at least one hetero atom of O, N, S, Si, or P; or a fused ring group of a C₃-C₆₀ aliphatic ring and a C₆-C₆₀ aromatic ring, and the R′ and R″ are bonded to each other to form a ring,
5) L₁ is selected from the group consisting of a single bond; a C₆-C₆₀ arylene group; a fluorenylene group; a C₂-C₆₀ heterocyclic group including at least one hetero atom of O, N, S, Si, or P; or a fused ring group of a C₃-C₆₀ aliphatic ring and a C₆-C₆₀ aromatic ring, and
6) Ar₁ is selected from the group consisting of a C₅-C₆₀ aryl group; a fluorenyl group; a C₂-C₆₀ heterocyclic group including at least one hetero atom of O, N, S, Si, or P; or a fused ring group of a C₃-C₆₀ aliphatic ring and a C₅-C₆₀ aromatic ring,
in Formula B,
1) 1 is an integer of from 0 to 5, m is an integer of from 0 to 4, each of y and z is an integer from 0 to 4, where y+z is not 0,
2) Rₐ and R_{b} are respectively and independently selected from the group consisting of deuterium; tritium; a halogen; a cyano group; a nitro group; a C₆-C₆₀ aryl group; a fluorenyl group; a C₂-C₆₀ heterocyclic group including at least one hetero atom of O, N, S, Si, or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; or a C₆-C₃₀ aryloxy group, and the plurality of Rₐs and the plurality of R_{b}s are bonded to each other to form rings, respectively,
in Formula C,
1) n is 1 or 2,
2) Ar₂ is a radical of a compound represented by Formula A or a radical of a compound represented by Formula B,
3) each of Ar₃ and Ar₄ is independently selected from the group consisting of a C₆-C₆₀ aryl group; a fluorenyl group; a C₂-C₆₀ heterocyclic group including at least one hetero atom of O, N, S, Si, or P; or a fused ring group of a C₃-C₆₀ aliphatic ring and a C₆-C₆₀ aromatic ring,
4) each of L₂ to L₄ is independently selected from the group consisting of a single bond; a C₆-C₆₀ arylene group; a fluorenylene group; or a C₂-C₆₀ heterocyclic group including at least one hetero atom of O, N, S, Si, or P, and
in Formula D,
1) o is an integer from 1 to 4,
2) each of Ar₅ to Ar₈ is independently selected from the group consisting of a C₆-C₆₀ aryl group; a fluorenyl group; a C₂-C₆₀ heterocyclic group including at least one hetero atom of O, N, S, Si, or P; or a fused ring group of a C₃-C₆₀ aliphatic ring and a C₆-C₆₀ aromatic ring,
3) each of L₅ to L₉ is independently selected from the group consisting of a single bond; a C₆-C₆₀ arylene group; a fluorenylene group; a C₂-C₆₀ heterocyclic group including at least one hetero atom of O, N, S, Si, or P; or a fused ring group of a C₃-C₆₀ aliphatic ring and a C₆-C₆₀ aromatic ring, wherein L₉ is a radical of a compound represented by Formula A or a radical of a compound represented by Formula B, and
in Formulas A to D,
each of the aryl group, the fluorenyl group, the heterocyclic group, the fused ring group, the alkyl group, the alkenyl group, the alkynyl group, the alkoxyl group, the aryloxy group, the arylene group, and the fluorenylene group is further substituted with one or more substituents selected from the group consisting of deuterium; a nitro group; a nitrile group; a halogen group; an amino group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₅ aryl group; a C₆-C₂₅ aryl group substituted with deuterium; a fluorenyl group; a C₂-C₂₀ heterocyclic group; a C₃-C₂₀ cycloalkyl group; a C₇-C₂₀ arylalkyl group; or a C₈-C₂₀ arylalkenyl group, wherein the further substituted substituents are bonded to form a ring, and
each of the further substituted substituents is further substituted with one or more substituents selected from the group consisting of deuterium; a nitro group; a nitrile group; a halogen group; an amino group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₅ aryl group; a C₆-C₂₅ aryl group substituted with deuterium; a fluorenyl group; a C₂-C₂₀ heterocyclic group; a C₃-C₂₀ cycloalkyl group; a C₇-C₂₀ arylalkyl group; or a C₈-C₂₀ arylalkenyl group, wherein the substituents are bonded to form a ring.

14. The device of claim 11, wherein the first hole transport layer comprises a first doping material-doped layer doped with the first doping material and a first doping material undoped layer not doped with the first doping material,
the first doping material-doped layer comprises the first compound and 5 to 15 parts by weight of the first doping material with respect to 100 parts by weight of the first compound,
the second hole transport layer comprises a second doping material-doped layer doped with the second doping material and a second doping material undoped layer not doped with the second doping material,
the second doping material-doped layer comprises the second compound and 5 to 15 parts by weight of the second doping material with respect to 100 parts by weight of the second compound,
the third hole transport layer comprises a third doping material-doped layer doped with a third doping material and a third doping material undoped layer not doped with the third doping material, and
the third doping material-doped layer comprises the third compound and 5 to 15 parts by weight of the third doping material with respect to 100 parts by weight of the third compound.

15. The device of claim 13, wherein the first hole transport layer comprises a first doping material-doped layer doped with the first doping material and a first doping material undoped layer not doped with the first doping material, and
the first doping material-doped layer comprises at least one of the first compound or the fourth compound, and comprises 5 to 15 parts by weight of the first doping material with respect to 100 parts by weight of a total amount of the first compound and the fourth compound.

16. The device of claim 1, wherein the organic material layer further comprises a fourth stack,
wherein the fourth stack comprises a fourth hole transport region, a fourth emission layer, and a fourth electron transport region,
the fourth hole transport region comprises a fourth hole transport layer and a fourth auxiliary emission layer,
the fourth hole transport layer or the fourth auxiliary emission layer comprises at least one of a fifth compound or a sixth compound,
the fifth compound is represented by Formula 1,
the sixth compound comprises a radical of a compound represented by the following Formula A or Formula B and is represented by the following Formula C or Formula D,
the thickness of the fourth hole transport layer ranges from 250 Å to 700 Å,
10% to 50% of the thickness of the fourth hole transport layer is doped with a fourth doping material,
[Formulas A, B, C, and D] 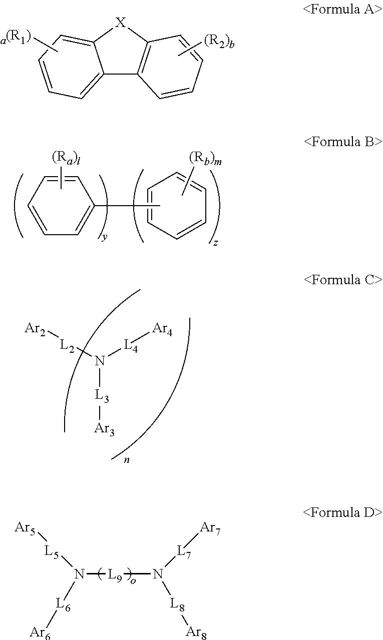
wherein, in Formula A,
1) each of a and b is independently an integer from 0 to 4,
2) X is O, S, CR′R″, or N-L₁-Ar₁,
3) R₁ and R₂ are respectively and independently selected from the group consisting of deuterium; tritium; a halogen; a cyano group; a nitro group; a C₆-C₆₀ aryl group; a fluorenyl group; a C₂-C₆₀ heterocyclic group including at least one hetero atom of O, N, S, Si, or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; or a C₆-C₃₀ aryloxy group, and the plurality of R₁s and the plurality of R₂s are bonded to form rings, respectively,
4) R′ and R″ are respectively and independently selected from the group consisting of hydrogen; deuterium; a C₆-C₆₀ aryl group; a fluorenyl group; a C₂-C₆₀ heterocyclic group including at least one hetero atom of O, N, S, Si, or P; or a fused ring group of a C₃-C₆₀ aliphatic ring and a C₆-C₆₀ aromatic ring, and the R′ and R″ are bonded to each other to form a ring,
5) L₁ is selected from the group consisting of a single bond; a C₆-C₆₀ arylene group; a fluorenylene group; a C₂-C₆₀ heterocyclic group including at least one hetero atom of O, N, S, Si, or P; or a fused ring group of a C₃-C₆₀ aliphatic ring and a C₆-C₆₀ aromatic ring, and
6) Ar₁ is selected from the group consisting of a C₆-C₆₀ aryl group; a fluorenyl group; a C₂-C₆₀ heterocyclic group including at least one hetero atom of O, N, S, Si, or P; or a fused ring group of a C₃-C₆₀ aliphatic ring and a C₆-C₆₀ aromatic ring,
in Formula B,
1) 1 is an integer of from 0 to 5, m is an integer of from 0 to 4, each of y and z is an integer from 0 to 4, where y+z is not 0,
2) Rₐ and R_{b} are respectively and independently selected from the group consisting of deuterium; tritium; a halogen; a cyano group; a nitro group; a C₆-C₆₀ aryl group; a fluorenyl group; a C₂-C₆₀ heterocyclic group including at least one hetero atom of O, N, S, Si, or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; or a C₆-C₃₀ aryloxy group, and the plurality of Rₐs and the plurality of R_{b}s are bonded to form rings, respectively,
in Formula C,
1) n is 1 or 2,
2) Ar₂ is a radical of a compound represented by Formula A or a radical of a compound represented by Formula B,
3) each of Ar₃ and Ar₄ is independently selected from the group consisting of a C₆-C₆₀ aryl group; a fluorenyl group; a C₂-C₆₀ heterocyclic group including at least one hetero atom of O, N, S, Si, or P; or a fused ring group of a C₃-C₆₀ aliphatic ring and a C₆-C₆₀ aromatic ring,
4) each of L₂ to L₄ is independently selected from the group consisting of a single bond; a C₆-C₆₀ arylene group; a fluorenylene group; or a C₂-C₆₀ heterocyclic group including at least one hetero atom of O, N, S, Si, or P, and
in Formula D,
1) o is an integer from 1 to 4,
2) each of Ar₅ to Are is independently selected from the group consisting of a C₆-C₆₀ aryl group; a fluorenyl group; a C₂-C₆₀ heterocyclic group including at least one hetero atom of O, N, S, Si, or P; or a fused ring group of a C₃-C₆₀ aliphatic ring and a C₆-C₆₀ aromatic ring,
3) each of L₅ to L₉ is independently selected from the group consisting of a single bond; a C₆-C₆₀ arylene group; a fluorenylene group; a C₂-C₆₀ heterocyclic group including at least one hetero atom of O, N, S, Si, or P; or a fused ring group of a C₃-C₆₀ aliphatic ring and a C₆-C₆₀ aromatic ring, wherein L₉ is a radical of a compound represented by the Formula A or a radical of a compound represented by the Formula B, and
in Formulas A to D,
each of the aryl group, the fluorenyl group, the heterocyclic group, the fused ring group, the alkyl group, the alkenyl group, the alkynyl group, the alkoxyl group, the aryloxy group, the arylene group, and the fluorenylene group is further substituted with one or more substituents selected from the group consisting of deuterium; a nitro group; a nitrile group; a halogen group; an amino group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₅ aryl group; a C₆-C₂₅ aryl group substituted with deuterium; a fluorenyl group; a C₂-C₂₀ heterocyclic group; a C₃-C₂₀ cycloalkyl group; a C₇-C₂₀ aryl alkyl group; or a C₈-C₂₀ aryl alkenyl group, wherein the further substituted substituents are bonded to each other to form a ring, and
each of the further substituted substituents is further substituted with one or more substituents selected from the group consisting of deuterium; a nitro group; a nitrile group; a halogen group; an amino group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₅ aryl group; a C₆-C₂₅ aryl group substituted with deuterium; a fluorenyl group; a C₂-C₂₀ heterocyclic group; a C₃-C₂₀ cycloalkyl group; a C₇-C₂₀ arylalkyl group; or a C₈-C₂₀ arylalkenyl group, wherein the substituents are bonded to each other to form a ring.

17. The device of claim 1, wherein at least one of the first emission layer, the second emission layer, or the third emission layer is a blue light emission layer.

18. The device of claim 1, wherein each of the first emission layer, the second emission layer, and the third emission layer is a blue light emission layer.

19. The device of claim 1, wherein one or two of the first emission layer, the second emission layer, and the third emission layer are blue light emission layers, and one or two of the first emission layer, the second emission layer, and the third emission layer may be green light emission layers.

20. The device of claim 1, wherein two emission layers of the first emission layer, the second emission layer, and the third emission layer are blue light emission layers, and the remaining one emission layer of the first emission layer, the second emission layer, and the third emission layer is a green light emission layer.

21. The device of claim 20, wherein the green light emission layer is positioned between the two blue light emission layers.

22. The device of claim 1, wherein at least one of the first emission layer, the second emission layer, or the third emission layer is a multi-emission layer emitting green light and blue light.

23. The device of claim 16, wherein three light emission layers of the first to fourth emission layers are blue light emission layers, and the remaining one emission layer is a green light emission layer.

24. The device of claim 16, wherein the fourth hole transport layer comprises the fifth compound.
